(19) United States
(12) Patent Application Publication
(10) Pub. No.: US 2023/0271949 A1
(43) Pub. Date: Aug. 31, 2023

(54) GLP-1R MODULATING COMPOUNDS

(71) Applicant: ASCLETIS BIOSCIENCE CO., LTD., Hangzhou (CN)

(72) Inventors: Bin Liang, Shanghai (CN); Yacheng Yang, Shanghai (CN); Peng Wang, Shanghai (CN); Bailing Yang, Shanghai (CN); Yang Lai, Shanghai (CN); Jinzi J. Wu, Shanghai (CN)

(21) Appl. No.: 18/056,836

(22) Filed: Nov. 18, 2022

Related U.S. Application Data
(60) Provisional application No. 63/268,622, filed on Feb. 28, 2022.
(60) Provisional application No. 63/366,565, filed on Jun. 17, 2022.

(51) Int. Cl. C07D 405/14; C07D 401/10; C07D 401/14; C07D 417/14; C07D 487/04; C07D 471/08; C07D 471/04; C07D 473/32; C07D 409/14; C07D 413/14; C07D 495/04; C07D 513/04; C07D 498/04

(52) U.S. Cl. CPC C07D 405/14; C07D 401/10; C07D 401/14; C07D 417/14; C07D 487/04; C07D 471/08; C07D 471/04; C07D 473/32; C07D 409/14; C07D 413/14; C07D 495/04; C07D 513/04; C07D 498/04

(57) ABSTRACT

The present application describes GLP-IR modulating compounds that are useful for treating GLP-IR-mediated diseases or conditions.

### RELATED APPLICATIONS

This application claims priority of U.S. Provisional Application No. 63/268,622, filed on Feb. 28, 2022, and U.S. Provisional Application No. 63/366,565, filed on Jun. 17, 2022, both of which are herein incorporated by reference in their entirety.

### FIELD

The present application generally relates to compounds that bind to and act as agonists or modulators of the glucagon-like peptide-1 receptor (GLP-1R), as well as the use of such compounds for the treatment and/or prevention of GLP-1R-mediated diseases and conditions.

### BACKGROUND

Glucagon-like peptide-1 (GLP-1) is a peptide hormone that is secreted from the enteroendocrine cells in the gut in response to a meal. GLP-1 is believed to play a role in regulation of post-prandial glycemia, via directly augmenting meal-induced insulin secretion from the pancreatic beta-cells, as well as in promoting satiety by delaying the transit of food through the gut. GLP-1 mediates intracellular signaling via the GLP-1 receptor (GLP-1R) which belongs to a family of G-protein coupled receptors that are present on the cell membrane and can result in accumulation of the secondary messenger cyclic adenosine monophosphate (cAMP) upon activation. Non-alcoholic steatohepatitis (NASH) can be associated with features of metabolic syndrome, including obesity, type 2 diabetes, insulin resistance and cardiovascular disease.

GLP-1R agonists are currently being investigated in connection with diabetes, obesity, and NASH. GLP-1R agonists include peptides, such as exenatide, liraglutide, and dulaglutide, that have been approved for the management of type 2 diabetes. Such peptides are predominantly administered by subcutaneous injection. Oral GLP-1 agonists are also under investigation for treatment of type 2 diabetes. Some GLP-1R agonists, such as liraglutide, dulaglutide, and exenatide, are resistant to rapid degradation by dipeptidyl peptidase 4, resulting in longer half-lives than endogenous GLP-1.

There remains a need for compounds, such as agonists of GLP-1R, with desirable therapeutic properties, metabolic properties, and/or easy administration in the treatment of GLP-1R-mediated diseases and conditions.

### SUMMARY

One aspect of the present application relates to a compound of Formula I-A-1:

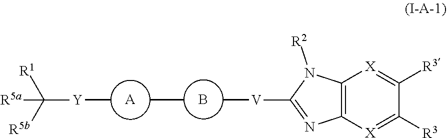

or a pharmaceutically acceptable salt thereof, wherein
R¹ is C₁₋₆ alkyl, C₁₋₆ haloalkyl, C₂₋₆ alkenyl, C₂₋₆ alkynyl, C₃₋₁₀ cycloalkyl, heterocyclyl, C₆₋₁₀ aryl, 5-10 membered heteroaryl, —S—R^{1b}, —S(O)R^{1b}, —S(O)(NH)R^{1b}, —S(O)₂R^{1b}, —S(O)₂N(R^{1b})(R^{1c}), —S(O)(NR^{1b})R^{1c}, —C(O)N(R^{1b})(R^{1c}), —C(O)R^{1b}, or —C(O)OR^{1c}, wherein the alkyl, haloalkyl, cycloalkyl, aryl, 5-10 membered heteroaryl, or heterocyclyl is each optionally substituted with one to four Z¹ and wherein any two Z¹ can be combined with the atoms to which they are attached to form a C₃₋₁₀ cycloalkyl or heterocyclyl or C₆₋₁₀ aryl or 5-10 membered heteroaryl;
Ring A is C₃₋₁₀ cycloalkyl, heterocyclyl, C₆₋₁₀ aryl or 5-10 membered heteroaryl, which is each optionally substituted with one to four Z^{1a};
Ring B is C₃₋₁₀ cycloalkyl, heterocyclyl, C₆₋₁₀ aryl or 5-10 membered heteroaryl, which is each optionally substituted with one to four R⁴;
Ring A and Ring B each contains zero to three double bonds;
R² is H, C₁₋₆ alkyl, —C₁₋₆ alkyl-alkoxy, —C₁₋₆ alkyl-cycloalkoxy, C₁₋₆ haloalkyl, C₂₋₆ alkenyl, C₂₋₆ alkynyl, C₃₋₁₀ cycloalkyl, heterocyclyl, C₆₋₁₀ aryl, 5-10 membered heteroaryl, —CN, —OR^{2a}, —S—R^{2a}, —S(O)R^{2a}, —S(O)(NH)R^{2a}, —S(O)₂R^{2a}, —S(O)₂N(R^{2a})(R^{2b}), or —S(O)(NR^{2a})R^{2b}, wherein the alkyl, alkoxy, cycloalkoxy, haloalkyl, cycloalkyl, heterocyclyl, aryl, or 5-10 membered heteroaryl in each instance is optionally substituted with one to four Z¹ and wherein any two Z¹ can be combined with the atoms to which they are attached to form a C₃₋₁₀ cycloalkyl or heterocyclyl or C₆₋₁₀ aryl or 5-10 membered heteroaryl;
X is each independently —N═, —C(H)═, or —C(R⁸)═;
each R³ and R^{3′} is independently H, C₁₋₆ alkyl, C₁₋₆ haloalkyl, C₂₋₆ alkenyl, C₂₋₆ alkynyl, halogen, C₃₋₁₀ cycloalkyl, heterocyclyl, C₆₋₁₀ aryl, 5-10 membered heteroaryl, —CN, —NO₂, —OR^{3a}, —C(O)R^{3a}, —CH₂C(O)OR^{3a}, —C(O)OR^{3a}, —C(O)N(R^{3a})(R^{3b}), —N(R^{3a})C(O)R^{3b}, —N(R^{3a})C(O)OR^{3b}, —N(R^{3a})C(O)N(R^{3b})₂, —C(O)NHS(O)₂R^{3a}, —C(O)NR^{3a}S(O)₂R^{3b}, —C(O)NR^{3a}S(O)₂NR^{3b}R^{3c}, —C(O)NR^{3a}—S(O)(═NR^{3b})R^{3c}, —S(O)₂R^{3a}, —S(O)₂OR^{3a}, —S(O)₂N(R^{3a})(R^{3b}), —N(R^{3a})S(O)₂R^{3b}, —S(O)₂NHC(O)R^{3a}, —S(O)(═NR^{3a})R^{3b}, —S(O)(═NR^{3a})NR^{3b}, —S(═NR^{3a})(═NR^{3b})R^{3c}, —P(O)(OR^{3a})(R^{3b}), —P(O)(OR^{3a})(OR^{3b}), —B(OR^{3a})(OR^{3b}), or —O—C₁₋₆alkyl-C(O)OR^{3a}, wherein the alkyl, haloalkyl, alkenyl, alkynyl, cycloalkyl, heterocyclyl, aryl, or 5-10 membered heteroaryl in each instance is optionally substituted with one to four R^{3d};
each R^{3a}, R^{3b}, and R^{3c} is independently H, C₁₋₆ alkyl, C₆₋₁₀ aryl, 5-10 membered heteroaryl, C₁₋₆ haloalkyl, C₂₋₈ alkoxyalkyl, —C₁₋₄ alkyl-N(R^{9a})(R^{9b}), —C₁₋₄ alkyl-N(R^{9a})C(O)—O—C₁₋₄ alkyl-OP(O)(OR^{9c})₂, —C₁₋₄ alkyl-C(O)N(R^{9a})(R^{9b}), —C₁₋₄ alkyl-O—C(O)—O—C₁₋₄ alkyl, —C₁₋₄ alkyl-O—C(O)—C₁₋₄ alkyl-N(R^{9a})(R^{9b}), —C₁₋₄ alkyl-O—C(O)—C₁₋₄ alkyl-OP(O)(OR^{9c})₂, —C₁₋₄ alkyl-C₃₋₈ cycloalkyl, —C₁₋₄ alkyl-heterocyclyl, C₂₋₆ alkenyl, C₂₋₆ alkynyl, C₃₋₁₀ cycloalkyl, heterocyclyl, —CH₂CH(N(R^{9a})₂)C(O)OR^{9b}, —P(O)(OR^{9c})₂, —OP(O)(OR^{9c})₂, —CH₂P(O)(OR^{9c})₂, —CH₂OP(O)(OR^{9c})₂, —OCH₂P(O)(OR^{9c})₂, —C(O)OCH₂P(O)(OR^{9c})₂, —P(O)(R^{9c})(OR^{9d}), —OP(O)(R^{9c})(OR^{9d}), —CH₂P(O)(R^{9c})(OR^{9d}), —OCH₂P(O)(R^{9c})(OR^{9d}), —C(O)OCH₂P(O)(R^{9c})(OR^{9d}), —P(O)(N(R^{9c})₂)₂, —OP(O)(N(R^{9c})₂)₂, —CH₂P(O)(N(R^{9c})₂)₂, —OCH₂P(O)(N(R^{9c})₂)₂, —C(O)OCH₂P(O)(N(R^{9c})₂)₂, —P(O)(N(R^{9c})₂)(OR^{9d}), —OP(O)(N(R^{9c})₂)(OR^{9d}), —CH₂P(O)(N(R^{9c})₂)(OR^{9d}), —OCH₂P(O)(N(R^{9c})₂)(OR^{9d}), —C(O)OCH₂P(O)(N(R^{9c})₂)(OR^{9d}), —P(O)(R^{9c})(N(R^{9d})₂), —OP(O)(R^{9c})(N(R^{9d})₂), —CH₂P(O)(R^{9c})(N(R^{9d})₂), —OCH₂P(O)(R^{9c})(N(R^{9d})₂), —C(O)OCH₂P(O)(R^{9c})(N(R^{9d})₂), C₁₋₆ alkyl-heterocyclyl, C₁₋₆ alkyl-aryl or C₁₋₆ alkyl-(5-10 membered) heteroaryl, wherein the alkyl, alkenyl, cycloalkyl, heterocyclyl, aryl, or 5-10 membered heteroaryl in each instance is optionally substituted with one to four Z^{1b};
each R⁴ is independently H, —OH, CH₂OH, C₁₋₉ alkyl, C₁₋₈ haloalkyl, C₁₋₆ haloalkoxy, C₂₋₆ alkoxyalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —NO₂, —CN, —N₃, —O—R^{4a}, —C(O)R^{4a}, —C(O)O—R^{4a}, —C(O)N(R^{4a})(R^{4b}), —N(R^{4a})(R^{4b}), —N(R^{4a})₂(R^{4b})⁺, —N(R^{4a})—C(O)R^{4b}, —N(R^{4a})C(O)O(R^{4b}), —N(R^{4a})C(O)N(R^{4b})(R^{4c}), —N(R^{4a})S(O)₂(R^{4b}), —N(R^{4a})S(O)₂—N(R^{4b})(R^{4c}), —N(R^{4a})S(O)₂O(R^{4b}), —OC(O)R^{4a}, —OC(O)OR^{4a}, —OC(O)—N(R^{4a})(R^{4b}), —S—R^{4a}, —S(O)R^{4a}, —S(O)(NH)R^{4a}, —S(O)₂R^{4a}, —S(O)₂N(R^{4a})(R^{4b}), —S(O)(NR^{4a})R^{4b}, or —Si(R^{4a})₃, wherein the alkyl, haloalkyl, alkenyl, alkynyl, cycloalkyl, heterocyclyl, aryl, or 5-10 membered heteroaryl in each instance is optionally substituted with one to four Z^{1b}, or two R⁴ groups attached to adjacent ring atoms are combined with the atoms to which they are attached to form a C₅₋₁₀ cycloalkyl or heterocyclyl, which is each optionally substituted with one to four Z^{1b};
each R^{5a} and R^{5b} is independently H, C₁₋₆ alkyl, C₁₋₆ alkyloxy, C₁₋₆ haloalkyl, C₂₋₆ alkoxyalkyl, halogen, C₃₋₁₀ cycloalkyl, heterocyclyl, —C₁₋₆ alkyl-N(R^{9a})(R^{9b}), —CN, —OR^{5a1}, or —N(R^{5a1})(R^{5a2}); or R^{5a} and R^{5b} are combined with the atoms to which they are attached to form a C₃₋₁₀ cycloalkyl or heterocyclyl, which is each optionally substituted with one to four R^{5a3}; or R^{5a} or R^{5b} can be combined with the substituents on Ring A to form a C₅₋₁₀ cycloalkyl or heterocyclyl, which is each optionally substituted with one to four R^{5a3};
each R^{5a1} and R^{5a2} is independently H, C₁₋₆ alkyl, C₂₋₆ alkenyl, C₂₋₆ alkynyl, C₃₋₁₀ cycloalkyl, heterocyclyl, C₆₋₁₀ aryl, or 5-10 membered heteroaryl, wherein the cycloalkyl, heterocyclyl, aryl, or 5-10 membered heteroaryl in each instance is optionally substituted with one to four R^{5a4};
V is —C(O)—, —O—, —N(R^{6a})—, or —C(R^{6b})(R^{6c})—;
R^{6a} is H, C₁₋₆ alkyl, C₃₋₁₀ cycloalkyl, heterocyclyl, —S(O)₂R^{6a1}, or —S(O)₂N(R^{6a1})(R^{6a2}), wherein the cycloalkyl or heterocyclyl in each instance is optionally substituted with C₁₋₆ alkyl, F, or —CN;
each R^{6b} and R^{6c} is independently H, C₁₋₆ alkyl, C₁₋₆ haloalkyl, C₂₋₆ alkoxyalkyl, halogen, C₃₋₁₀ cycloalkyl, heterocyclyl, —C₁₋₆ alkyl-N(R^{9a})(R^{9b}), —CN, —OR^{6c1}, or —N(R^{6c2})(R^{6c3}), wherein the alkyl, cycloalkyl, or heterocyclyl in each instance is optionally substituted with one to four R^{6b1}; or R^{6b} and R^{6c} are combined with the atom to which they are attached to form C₃₋₁₀ cycloalkyl or heterocyclyl, which is each optionally substituted with one to four R^{6b1}; or R^{6a} or R^{6c} is combined with one R⁴ group and the atoms to which they are attached to form a C₅₋₁₀ cycloalkyl or heterocyclyl, which in each instance is optionally substituted with one to four R¹⁰;
Y is —N(R⁷)—, —O—, —S—, —S(O)—, —S(O)₂—, —S(O)(═NH)—, or —S(O)(═NR⁷)—;
each R^{3d}, R^{5a3}, R^{5a4}, R^{6b1}, and R¹⁰ is independently H, C₁₋₆ alkyl, C₁₋₆ haloalkyl, C₁₋₆ alkoxy, C₁₋₆ haloalkoxy, C₂₋₆ alkoxyalkyl, C₂₋₆ alkenyl, C₂₋₆ alkynyl, halogen, C₃₋₁₀ cycloalkyl, heterocyclyl, C₆₋₁₀ aryl, 5-10 membered heteroaryl, oxo, —OH, —CN, CO₂R^{3e}, —NO₂, or —C(O)N(R^{2a})(R^{2b}), wherein the heterocyclyl or 5-10 membered heteroaryl in each instance is optionally substituted with C₁₋₆ alkyl, C₁₋₆ haloalkyl, or C₁₋₆ haloalkoxy; and
each R^{6a1}, R^{6a2}, R^{6c1}, R^{6c2}, R^{6c3} and R⁷ is independently H, C₁₋₆ alkyl or C₃₋₁₀ cycloalkyl;
each R⁸ is independently C₁₋₉ alkyl, C₁₋₈ haloalkyl, C₂₋₆ alkenyl, C₂₋₆ alkynyl, halogen, oxo, —OH, —CN, CO₂R^{3e}, —NO₂, —NH₂, —N₃, —SH, —O(C₁₋₉ alkyl), —O(C₁₋₈ haloalkyl), —NH(C₁₋₉ alkyl), —NH(C₁₋₈ haloalkyl), —N(C₁₋₉ alkyl)₂, —N(C₁₋₈ haloalkyl)₂;
each R^{9a} and R^{9b} is independently H, C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —NO₂, —CN, —N₃, —O—R^{12a}, —C(O)R^{12a}, —C(O)O—R^{12a}, —C(O)N(R^{12a})(R^{12b}), —N(R^{12a})(R^{12b}), —N(R^{12a})₂(R^{12b})⁺, —N(R^{12a})—C(O)R^{12b}, —N(R^{12a})C(O)O(R^{12b}), —N(R^{12a})C(O)N(R^{12b})(R^{12c}), —N(R^{12a})S(O)₂(R^{12b}), —N(R^{12a})S(O)₂—N(R^{12b})(R^{12c}), —N(R^{12a})S(O)₂O(R^{12b}), —OC(O)R^{12a}, —OC(O)OR^{12a}, —OC(O)—N(R^{12a})(R^{12b}), —S—R^{12a}, —S(O)R^{12a}, —S(O)(NH)R^{12a}, —S(O)₂R^{12a}, —S(O)₂N(R^{12a})(R^{12b}), —S(O)(NR^{12a})R^{12b}, or —Si(R^{12a})₃; or R^{9a} and R^{9b} together form a 3-10 membered heterocyclyl optionally substituted with C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, halogen, C₃₋₁₅ cycloalkyl, heterocyclyl, C₆₋₁₀ aryl, heteroaryl, oxo, —NO₂, —CN, —N₃, —O—R^{12a}, —C(O)R^{12a}, —C(O)O—R^{12a}, —C(O)N(R^{12a})(R^{12b}), —N(R^{12a})(R^{12b}), —N(R^{12a})₂(R^{12b})⁺, —N(R^{12a})—C(O)R^{12b}, —N(R^{12a})C(O)O(R^{12b}), —N(R^{12a})C(O)N(R^{12b})(R^{12c}), —N(R^{12a})S(O)₂(R^{12b}), —N(R^{12a})S(O)₂—N(R^{12b})(R^{12c}), —N(R^{12a})S(O)₂O(R^{12b}), —OC(O)R^{12a}, —OC(O)OR^{12a}, —OC(O)—N(R^{12a})(R^{12b}), —S—R^{12a}, —S(O)R^{12a}, —S(O)(NH)R^{12a}, —S(O)₂R^{12a}, —S(O)₂N(R^{12a})(R^{12b}), —S(O)(NR^{12a})R^{12b}, or —Si(R^{12a})₃;
each Z¹ is independently C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —NO₂, —N₃, —CN, —O—R^{12a}, —C(O)—R^{12a}, —C(O)O—R^{12a}, —C(O)—N(R^{12a})(R^{12b}), —N(R^{12a})(R^{12b}), —N(R^{12a})₂(R^{12b})⁺, —N(R^{12a})C(O)—R^{12b}, —N(R^{12a})C(O)O—R^{12b}, —N(R^{12a})C(O)N(R^{12b})(R^{12c}), —N(R^{12a})S(O)₂(R^{12b}), —NR^{12a}S(O)₂N(R^{12b})(R^{12c}), —NR^{12a}S(O)₂O(R^{12b}), —OC(O)R^{12a}, —OC(O)OR^{12a}, —OC(O)—N(R^{12a})(R^{12b}), —S—R^{12a}, —S(O)R^{12a}, —S(O)(NH)R^{12a}, —S(O)₂R^{12a}, —S(O)₂N(R^{12a})(R^{12b}), —S(O)(NR^{12a})R^{12b}, or —Si(R^{12a})₃, wherein the alkyl, haloalkyl, alkenyl, alkynyl, cycloalkyl, heterocyclyl, aryl, or 5-10 membered heteroaryl in each instance is optionally substituted with one to four Z^{1a};
each Z^{1a} is independently H, C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —NO₂, —CN, —N₃, —O—R^{12a}, —C(O)R^{12a}, —C(O)O—R^{12a}, —C(O)N(R^{12a})(R^{12b}), —N(R^{12a})(R^{12b}), —N(R^{12a})₂(R^{12b})⁺, —N(R^{12a})—C(O)R^{12b}, —N(R^{12a})C(O)O(R^{12b}), —N(R^{12a})C(O)N(R^{12b})(R^{12c}), —N(R^{12a})S(O)₂(R^{12b}), —N(R^{12a})S(O)₂—N(R^{12b})(R^{12c}), —N(R^{12a})S(O)₂O(R^{12b}), —OC(O)R^{12a}, —OC(O)OR^{12a}, —OC(O)—N(R^{12a})(R^{12b}), —S—R^{12a}, —S(O)R^{12a}, —S(O)(NH)R^{12a}, —S(O)₂R^{12a}, —S(O)₂N(R^{12a})(R^{12b}), —S(O)(NR^{12a})R^{12b}, or —Si(R^{12a})₃, wherein the alkyl, haloalkyl, alkenyl, alkynyl, cycloalkyl, heterocyclyl, aryl, or 5-10 membered heteroaryl in each instance is optionally substituted with one to four Z^{1b};
each Z^{1b} is independently H, C₁₋₉ alkyl, C₁₋₈ haloalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —OH, —CN, CO₂R^{3e}, —NO₂, —NH₂, —N₃, —SH, —O(C₁₋₉ alkyl), —O(C₁₋₈ haloalkyl), —O(C₂₋₆ alkenyl), —O(C₂₋₆ alkynyl), —O(C₃₋₁₅ cycloalkyl), —O(heterocyclyl), —O(C₆₋₁₀ aryl), —O(5-10 membered heteroaryl), —NH(C₁₋₉ alkyl), —NH(C₁₋₈ haloalkyl), —NH(C₂₋₆ alkenyl), —NH(C₂₋₆ alkynyl), —NH(C₃₋₁₅ cycloalkyl), —NH(heterocyclyl), —NH(C₆₋₁₀ aryl), —NH(5-10 membered heteroaryl), —N(cyclo-alkyl), —N(C₁₋₉ alkyl)₂, —N(C₁₋₈ haloalkyl)₂, —N(C₂₋₆ alkenyl)₂, —N(C₂₋₆ alkynyl)₂, —N(C₃₋₁₅ cycloalkyl)₂, —N(heterocyclyl)₂, —N(C₆₋₁₀ aryl)₂, —N(5-10 membered heteroaryl)₂, —N(C₁₋₉ alkyl)(C₁₋₈ haloalkyl), —N(C₁₋₉ alkyl)(C₂₋₆ alkenyl), —N(C₁₋₉ alkyl)(C₂₋₆ alkynyl), —N(C₁₋₉ alkyl)(C₃₋₁₅ cycloalkyl), —N(C₁₋₉ alkyl)(heterocyclyl), —N(C₁₋₉ alkyl)(C₆₋₁₀ aryl), —N(C₁₋₉ alkyl)(5-10 membered heteroaryl), —C(O)(C₁₋₉ alkyl), —C(O)(C₁₋₈ haloalkyl), —C(O)(C₂₋₆ alkenyl), —C(O)(C₂₋₆ alkynyl), —C(O)(C₃₋₁₅ cycloalkyl), —C(O)(heterocyclyl), —C(O)(C₆₋₁₀ aryl), —C(O)(5-10 membered heteroaryl), —C(O)O(C₁₋₉ alkyl), —C(O)O(C₁₋₈ haloalkyl), —C(O)O(C₂₋₆ alkenyl), —C(O)O(C₂₋₆ alkynyl), —C(O)O(C₃₋₁₅ cycloalkyl), —C(O)O(heterocyclyl), —C(O)O(C₆₋₁₀ aryl), —C(O)O(5-10 membered heteroaryl), —C(O)NH₂, —C(O)NH(C₁₋₉ alkyl), —C(O)NH(C₁₋₈ haloalkyl), —C(O)NH(C₂₋₆ alkenyl), —C(O)NH(C₂₋₆ alkynyl), —C(O)NH(C₃₋₁₅ cycloalkyl), —C(O)NH(heterocyclyl), —C(O)NH(C₆₋₁₀ aryl), —C(O)NH(5-10 membered heteroaryl), —C(O)N(C₁₋₉ alkyl)₂, —C(O)N(C₁₋₈ haloalkyl)₂, —C(O)N(C₂₋₆ alkenyl)₂, —C(O)N(C₂₋₆ alkynyl)₂, —C(O)N(C₃₋₁₅ cycloalkyl)₂, —C(O)N(heterocyclyl)₂, —C(O)N(C₆₋₁₀ aryl)₂, —C(O)N(5-10 membered heteroaryl)₂, —NHC(O)(C₁₋₉ alkyl), —NHC(O)(C₁₋₈ haloalkyl), —NHC(O)(C₂₋₆ alkenyl), —NHC(O)(C₂₋₆ alkynyl), —NHC(O)(C₃₋₁₅ cycloalkyl), —NHC(O)(heterocyclyl), —NHC(O)(C₆₋₁₀ aryl), —NHC(O)(5-10 membered heteroaryl), —NHC(O)O(C₁₋₉ alkyl), —NHC(O)O(C₁₋₈ haloalkyl), —NHC(O)O(C₂₋₆ alkenyl), —NHC(O)O(C₂₋₆ alkynyl), —NHC(O)O(C₃₋₁₅ cycloalkyl), —NHC(O)O(heterocyclyl), —NHC(O)O(C₆₋₁₀ aryl), —NHC(O)O(5-10 membered heteroaryl), —NHC(O)NH(C₁₋₉ alkyl), —NHC(O)NH(C₁₋₈ haloalkyl), —NHC(O)NH(C₂₋₆ alkenyl), —NHC(O)NH(C₂₋₆ alkynyl), —NHC(O)NH(C₃₋₁₅ cycloalkyl), —NHC(O)NH(heterocyclyl), —NHC(O)NH(C₆₋₁₀ aryl), —NHC(O)NH(5-10 membered heteroaryl), —NHS(O)(C₁₋₉ alkyl), —N(C₁₋₉ alkyl)(S(O)(C₁₋₉ alkyl), —S(C₁₋₉ alkyl), —S(C₁₋₈ haloalkyl), —S(C₂₋₆ alkenyl), —S(C₂₋₆ alkynyl), —S(C₃₋₁₅ cycloalkyl), —S(heterocyclyl), —S(C₆₋₁₀ aryl), —S(5-10 membered heteroaryl), —S(O)N(C₁₋₉ alkyl)₂, —S(O)(C₁₋₉ alkyl), —S(O)(C₁₋₈ haloalkyl), —S(O)(C₂₋₆ alkenyl), —S(O)(C₂₋₆ alkynyl), —S(O)(C₃₋₁₅ cycloalkyl), —S(O)(heterocyclyl), —S(O)(C₆₋₁₀ aryl), —S(O)(5-10 membered heteroaryl), —S(O)₂(C₁₋₉ alkyl), —S(O)₂(C₁₋₈ haloalkyl), —S(O)₂(C₂₋₆ alkenyl), —S(O)₂(C₂₋₆ alkynyl), —S(O)₂(C₃₋₁₅ cycloalkyl), —S(O)₂(heterocyclyl), —S(O)₂(C₆₋₁₀ aryl), —S(O)₂(5-10 membered heteroaryl), —S(O)(NH)(C₁₋₉ alkyl), —S(O)₂NH(C₁₋₉ alkyl), or —S(O)₂N(C₁₋₉ alkyl)₂, wherein the alkyl, cycloalkyl, heterocyclyl, aryl, or 5-10 membered heteroaryl in each instance is optionally substituted with one to three C₁₋₉ alkyl, C₁₋₈ haloalkyl, halogen, —OH, —NH₂, CO₂H, —O(C₁₋₉ alkyl), —O(C₁₋₈ haloalkyl), —O(C₃₋₁₅ cycloalkyl), —O(heterocyclyl), —O(aryl), —O(5-10 membered heteroaryl), —NH(C₁₋₉ alkyl), —NH(C₁₋₈ haloalkyl), —NH(C₃₋₁₅ cycloalkyl), —NH(heterocyclyl), —NH(aryl), —NH(5-10 membered heteroaryl), —N(C₁₋₉ alkyl)₂, —N(C₃₋₁₅ cycloalkyl)₂, —NHC(O)(C₁₋₈ haloalkyl), —NHC(O)(C₃₋₁₅ cycloalkyl), —NHC(O)(heterocyclyl), —NHC(O)(aryl), —NHC(O)(5-10 membered heteroaryl), —NHC(O)O(C₁₋₉ alkyl), —NHC(O)O(C₁₋₈ haloalkyl), —NHC(O)O(C₂₋₆ alkynyl), —NHC(O)O(C₃₋₁₅ cycloalkyl), —NHC(O)O(heterocyclyl), —NHC(O)O(aryl), —NHC(O)O(5-10 membered heteroaryl), —NHC(O)NH(C₁₋₉ alkyl), S(O)₂(C₁₋₉ alkyl), —S(O)₂(C₁₋₈ haloalkyl), —S(O)₂(C₃₋₁₅ cycloalkyl), —S(O)₂(heterocyclyl), —S(O)₂(aryl), —S(O)₂(5-10 membered heteroaryl), —S(O)(NH)(C₁₋₉ alkyl), —S(O)₂NH(C₁₋₉ alkyl), or —S(O)₂N(C₁₋₉ alkyl)₂;
each R^{1b}, R^{1c}, R^{2a}, R^{2b}, R^{4a}, R^{4b}, R^{4c}, R^{9c}, R^{9d}, R^{12a}, R^{12b}, and R^{12c} is independently H, —OH, C₁₋₉ alkyl, C₂₋₆ alkenyl, C₂₋₆ alkynyl, C₃₋₁₅ cycloalkyl, heterocyclyl, C₆₋₁₀ aryl, or 5-10 membered heteroaryl, wherein the alkyl, alkenyl, alkynyl, cycloalkyl, heterocyclyl, aryl, or 5-10 membered heteroaryl in each instance is optionally substituted with one to four Z^{1b}; and
each R^{3e} is independently H, C₁₋₆ alkyl, C₁₋₆ haloalkyl, —C₁₋₄ alkyl-N(R^{9a})(R^{9b}), —C₁₋₄ alkyl-C(O)N(R^{9a})(R^{9b}), —C₁₋₄ alkyl-O—C(O)—O—C₁₋₄ alkyl, —C₁₋₄ alkyl-O—C(O)—C₁₋₄ alkyl-N(R^{9a})(R^{9b}), —C₁₋₄ alkyl-C₃₋₈ cycloalkyl, —C₁₋₄ alkyl-heterocyclyl, C₃₋₁₀ cycloalkyl, heterocyclyl, C₆₋₁₀ aryl, 5-10 membered heteroaryl, —P(O)(OR^{9c})₂, —CH₂P(O)(OR^{9c})₂, —OCH₂P(O)(OR^{9c})₂, —C(O)OCH₂P(O)(OR^{9c})₂, —P(O)(R^{9c})(OR^{9d}), —OP(O)(R^{9c})(OR^{9d}), —CH₂P(O)(R^{9c})(OR^{9d}), —C(O)OCH₂P(O)(R^{9c})(OR^{9d}), —P(O)(N(R^{9c})₂)₂, —CH₂P(O)(N(R^{9c})₂)₂, —C(O)OCH₂P(O)(N(R^{9c})₂)₂, —P(O)(N(R^{9c})₂)(OR^{9d}), —CH₂P(O)(N(R^{9c})₂)(OR^{9d}), —C(O)OCH₂P(O)(N(R^{9c})₂)(OR^{9d}), —P(O)(R^{9c})(N(R^{9d})₂), —CH₂P(O)(R^{9c})(N(R^{9d})₂), or —C(O)OCH₂P(O)(R^{9c})(N(R^{9d})₂), wherein the alkyl, alkenyl, cycloalkyl, heterocyclyl, aryl, or 5-10 membered heteroaryl in each instance is optionally substituted with one to four Z^{1b}, wherein each 5-10 membered heteroaryl has 5 to 12 ring members and has one to four heteroatoms, each is independently N, O, or S, and wherein each heterocyclyl has 3 to 12 ring members and has one to four heteroatoms, each is independently N, O, or S.

Another aspect of the present application relates to methods for treating GLP-1R mediated diseases or conditions with the compounds of the present application.

Another aspect of the present application relates to methods for making the compounds of the present application.

### DETAILED DESCRIPTION

### I. Definitions

Unless defined otherwise, all technical and scientific terms used herein have the same meaning as commonly understood by one of ordinary skill in the art. A dash at the front or end of a chemical group is a matter of convenience to indicate the point of attachment to a parent moiety; chemical groups may be depicted with or without one or more dashes without losing their ordinary meaning. A prefix such as “Cᵤ₋ᵥ” or “Cᵤ-Cᵥ” indicates that the following group has from u to v carbon atoms, where u and v are integers. For example, “C₁₋₆ alkyl” or “C₁-C₆ alkyl” indicates that the alkyl group has from 1 to 6 carbon atoms.

“Alkyl” is a monovalent or divalent linear or branched saturated hydrocarbon radical. For example, an alkyl group can have 1 to 10 carbon atoms (i.e., C₁₋₁₀ alkyl) or 1 to 8 carbon atoms (i.e., C₁₋₈ alkyl) or 1 to 6 carbon atoms (i.e., C₁₋₆ alkyl) or 1 to 4 carbon atoms (i.e., C₁₋₄ alkyl). Examples of alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-Propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, and octyl (—(CH₂)₇CH₃). Alkyl groups can be unsubstituted or substituted.

“Alkoxy” refers to the group —O-alkyl, where alkyl is as defined above. For example, C₁₋₄ alkoxy refers to an —O-alkyl group having 1 to 4 carbons. Alkoxy groups can be unsubstituted or substituted.

“Alkoxyalkyl” is an alkoxy group attached to an alkyl as defined above, such that the alkyl is divalent. For example, C₂₋₆ alkoxyalkyl includes —CH₂—OMe, —CH₂—O-iPr, —CH₂—CH₂—OMe, —CH₂—CH₂—O—CH₂—CH₃, and —CH₂—CH₂—O-tBu. Alkoxyalkyl groups can be unsubstituted or substituted.

“Alkenyl” is a monovalent or divalent linear or branched hydrocarbon radical with at least one carbon-carbon double bond. For example, an alkenyl group can have 2 to 8 carbon atoms (i.e., C₂₋₈ alkenyl) or 2 to 6 carbon atoms (i.e., C₂₋₆ alkenyl) or 2 to 4 carbon atoms (i.e., C₂₋₄ alkenyl). Examples of alkenyl groups include, but are not limited to, ethenyl (—CH═CH₂), allyl (—CH₂CH═CH₂), and —CH₂—CH═CH—CH₃. Alkenyl groups can be unsubstituted or substituted.

“Alkynyl” is a monovalent or divalent linear or branched hydrocarbon radical with at least one carbon-carbon triple bond. For example, an alkynyl group can have 2 to 8 carbon atoms (i.e., C₂₋₈ alkynyl) or 2 to 6 carbon atoms (i.e., C₂₋₆ alkynyl) or 2 to 4 carbon atoms (i.e., C₂₋₄ alkynyl). Examples of alkynyl groups include, but are not limited to, acetylenyl (—C≡CH), propargyl (—CH₂C≡CH), and —CH2-C≡C—CH₃. Alkynyl groups can be unsubstituted or substituted.

“Halogen” refers to fluoro (—F), chloro (—Cl), bromo (—Br) and iodo (—I).

“Haloalkyl” is an alkyl as defined herein, wherein one or more hydrogen atoms of the alkyl are independently replaced by a halogen, which may be the same or different, such that the alkyl is divalent. The alkyl group and the halogen can be any of those described above. In some embodiments, the haloalkyl defines the number of carbon atoms in the alkyl portion, e.g., C₁₋₄ haloalkyl includes CF₃, CH₂F, CHF₂, CH₂CF₃, CH₂CH₂CF₃, CCl₂CH₂CH₂CH₃, and C(CH₃)₂(CF₂H). Haloalkyl groups can be unsubstituted or substituted.

“Haloalkoxy” is an alkoxy as defined herein, wherein one or more hydrogen atoms of the alkyl in the alkyoxy are independently replaced by a halogen, which may be the same or different, such that the alkyl is divalent. The alkoxy group and the halogen can be any of those described above. In some embodiments, the haloalkoxy defines the number of carbon atoms in the alkyl portion, e.g., C₁₋₄ haloalkoxy includes OCF₃, OCH₂F, OCH₂CF₃, OCH₂CH₂CF₃, OCCl₂CH₂CH₂CH₃, and OC(CH₃)₂(CF₂H). Haloalkoxy groups can be unsubstituted or substituted.

“Cycloalkyl” is a monovalent or divalent single all carbon ring or a multiple condensed all carbon ring system wherein the ring in each instance is a non-aromatic saturated or unsaturated ring. For example, in some embodiments, a cycloalkyl group has 3 to 12 carbon atoms, 3 to 10 carbon atoms, 3 to 8 carbon atoms, 3 to 6 carbon atoms, 3 to 5 carbon atoms, or 3 to 4 carbon atoms. Exemplary single ring cycloalkyl groups include cyclopropyl, cyclobutyl, cyclopentyl, cyclohexyl, cyclohexenyl, cycloheptyl, cycloheptenyl, and cyclooctyl. Cycloalkyl also includes multiple condensed ring systems (e.g., ring systems comprising 2 rings) having about 7 to 12 carbon atoms. The rings of the multiple condensed ring system can be connected to each other via fused, spiro, or bridged bonds when allowed by valency requirements. Exemplary multiple ring cycloalkyl groups include octahydropentalene, bicyclo[2.2.1]heptane, bicyclo[2.2.2]octane, bicyclo[2.2.2]oct-2-ene, and spiro[2.5]octane. Cycloalkyl groups can be unsubstituted or substituted.

Alkylcycloalkyl” refers to an alkyl as defined herein, wherein one or more hydrogen atoms of the alkyl are independently replaced by a cycloalkyl group, which may be the same or different. The alkyl group and the cycloalkyl group can be any of those described above. In some embodiments, the number of carbon atoms in the alkyl and cycloalkyl portion can be designated separately, e.g., C₁₋₆ alkyl-C₃₋₁₂ cycloalkyl. Alkylcycloalkyl groups can be unsubstituted or substituted.

“Aryl” as used herein refers to a monovalent or divalent single all carbon aromatic ring or a multiple condensed all carbon ring system wherein the ring is aromatic. For example, in some embodiments, an aryl group has 6 to 20 carbon atoms, 6 to 14 carbon atoms, 6 to 12 carbon atoms, or 6 to 10 carbon atoms. Aryl includes a phenyl radical. Aryl also includes multiple condensed ring systems (e.g., ring systems comprising 2, 3 or 4 rings) having about 9 to 20 carbon atoms in which multiple rings are aromatic. The rings of the multiple condensed ring system can be connected to each other via fused bonds when allowed by valency requirements. It is also understood that when reference is made to a certain atom-range membered aryl (e.g., 6-10 membered aryl), the atom range is for the total ring atoms of the aryl. For example, a 6-membered aryl would include phenyl and a 10-membered aryl would include naphthyl. Non-limiting examples of aryl groups include, but are not limited to, phenyl, naphthyl, anthracenyl, and the like. Aryl groups can be unsubstituted or substituted.

“Alkylaryl” refers to an alkyl as defined herein, wherein one or more hydrogen atoms of the alkyl are independently replaced by an aryl group, which may be the same or different. The alkyl group and the aryl group can be any of those described above, such that the alkyl is divalent. In some embodiments, an alkylaryl group has 7 to 24 carbon atoms, 7 to 16 carbon atoms, 7 to 13 carbon atoms, or 7 to 11 carbon atoms. An alkylaryl group defined by the number of carbon atoms refers to the total number of carbon atoms present in the constitutive alkyl and aryl groups combined. For example, C₇ alkylaryl refers to benzyl, while C₁₁ alkylaryl includes 1-methylnaphthyl and n-pentylphenyl. In some embodiments the number of carbon atoms in the alkyl and aryl portion can be designated separately, e.g., C₁₋₆ alkyl-C₆₋₁₀ aryl. Non-limiting examples of alkylaryl groups include, but are not limited to, benzyl, 2,2-dimethylphenyl, n-pentylphenyl, 1-methylnaphthyl, 2-ethylnaphthyl, and the like. Alkylaryl groups can be unsubstituted or substituted.

“Heterocyclyl” or “heterocycle” or “heterocycloalkyl” as used herein refers to a single saturated or partially unsaturated non-aromatic ring or a non-aromatic multiple ring system that has at least one heteroatom in the ring (i.e., at least one annular (i.e., ring-shaped) heteroatom selected from oxygen, nitrogen, and sulfur). Unless otherwise specified, a heterocyclyl group has from 3 to about 20 annular atoms, for example from 3 to 12 annular atoms, for example from 4 to 12 annular atoms, 4 to 10 annular atoms, or 3 to 8 annular atoms, or 3 to 6 annular atoms, or 3 to 5 annular atoms, or 4 to 6 annular atoms, or 4 to 5 annular atoms. Thus, the term includes single saturated or partially unsaturated rings (e.g., 3, 4, 5, 6 or 7-membered rings) having from about 1 to 6 annular carbon atoms and from about 1 to 3 annular heteroatoms selected from the group consisting of oxygen, nitrogen and sulfur in the ring. The rings of the multiple condensed ring (e.g. bicyclic heterocyclyl) system can be connected to each other via fused, spiro and bridged bonds when allowed by valency requirements. Heterocycles include, but are not limited to, azetidine, aziridine, imidazolidine, morpholine, oxirane (epoxide), oxetane, thietane, piperazine, piperidine, pyrazolidine, piperidine, pyrrolidine, pyrrolidinone, tetrahydrofuran, tetrahydrothiophene, dihydropyridine, tetrahydropyridine, quinuclidine, 2-oxa-6-azaspiro[3.3]heptan-6-yl, 6-oxa-1-azaspiro[3.3]heptan-1-yl, 2-thia-6-azaspiro[3.3]heptan-6-yl, 2,6-diazaspiro[3.3]heptan-2-yl, 2-azabicyclo[3.1.0]hexan-2-yl, 3-azabicyclo[3.1.0]hexanyl, 2-azabicyclo[2.1.1]hexanyl, 2-azabicyclo[2.2.1]heptan-2-yl, 4-azaspiro[2.4]heptanyl, 5-azaspiro[2.4]heptanyl, and the like. Heterocyclyl groups can be unsubstituted or substituted.

“Alkylheterocyclyl” refers to an alkyl as defined herein, wherein one or more hydrogen atoms of the alkyl are independently replaced by a heterocyclyl group, which may be the same or different. The alkyl group and the heterocyclyl group can be any of those described above, such that the alkyl is divalent. In some embodiments, the number of atoms in the alkyl and heterocyclyl portion can be designated separately, e.g., C₁₋₆ alkyl-3 to 12 membered heterocyclyl having one to three heteroatoms each independently N, O, or S. Alkylheterocyclyl groups can be unsubstituted or substituted.

“5-10 membered heteroaryl” refers to a single aromatic ring that has at least one atom other than carbon in the ring, wherein the atom is selected from the group consisting of oxygen, nitrogen and sulfur; “5-10 membered heteroaryl” also includes multiple condensed ring systems that have at least one such aromatic ring, which multiple condensed ring systems are further described below. Thus, “5-10 membered heteroaryl” includes single aromatic rings of from about 1 to 6 carbon atoms and about 1-4 heteroatoms selected from the group consisting of oxygen, nitrogen and sulfur. The sulfur and nitrogen atoms may also be present in an oxidized form provided the ring is aromatic. Exemplary 5-10 membered heteroaryl ring systems include but are not limited to pyridyl, pyrimidinyl, oxazolyl or furyl. “5-10 membered heteroaryl” also includes multiple condensed ring systems (e.g., ring systems comprising 2, 3 or 4 rings) wherein a 5-10 membered heteroaryl group, as defined above, is condensed with one or more rings selected from 5-10 membered heteroaryls (to form for example 1,8-naphthyridinyl) and aryls (to form, for example, benzimidazolyl or indazolyl) to form the multiple condensed ring system. Thus, a 5-10 membered heteroaryl (a single aromatic ring or multiple condensed ring system) can have about 1-20 carbon atoms and about 1-6 heteroatoms within the 5-10 membered heteroaryl ring. For example, tetrazolyl has 1 carbon atom and 4 nitrogen heteroatoms within the ring. The rings of the multiple condensed ring system can be connected to each other via fused bonds when allowed by valency requirements. It is to be understood that the individual rings of the multiple condensed ring system may be connected in any order relative to one another. It is to be understood that the point of attachment for a 5-10 membered heteroaryl or 5-10 membered heteroaryl multiple condensed ring system can be at any suitable atom of the 5-10 membered heteroaryl or 5-10 membered heteroaryl multiple condensed ring system including a carbon atom and a heteroatom (e.g., a nitrogen). It also to be understood that when a reference is made to a certain atom-range membered (e.g., a 5-10 membered heteroaryl), the atom range is for the total ring atoms of the 5-10 membered heteroaryl and includes carbon atoms and heteroatoms. It is also to be understood that the rings of the multiple condensed ring system may include an aryl ring fused to a heterocyclic ring with saturated or partially unsaturated bonds (e.g., 3, 4, 5, 6 or 7-membered rings) having from about 1 to 6 annular carbon atoms and from about 1 to 3 annular heteroatoms selected from the group consisting of oxygen, nitrogen and sulfur in the ring. For example, a 5-10 membered heteroaryl includes thiazolyl and a 5-10 membered heteroaryl includes quinolinyl. Exemplary 5-10 membered heteroaryls include but are not limited to pyridyl, pyrrolyl, pyrazinyl, pyrimidinyl, pyridazinyl, pyrazolyl, thienyl, indolyl, imidazolyl, oxazolyl, isoxazolyl, thiazolyl, furyl, oxadiazolyl, thiadiazolyl, quinolyl, isoquinolyl, benzothiazolyl, benzoxazolyl, indazolyl, quinoxalyl, quinazolyl, benzofuranyl, benzimidazolyl, thianaphthenyl, pyrrolo[2,3-b]pyridinyl, quinazolinyl-4(3H)-one, triazolyl, and tetrazolyl. 5-10 membered heteroaryl groups can be unsubstituted or substituted.

“Alkylheteroaryl” refers to an alkyl as defined herein, wherein one or more hydrogen atoms of the alkyl are independently replaced by a heteroaryl group, which may be the same or different, such that the alkyl is divalent. The alkyl group and the heteroaryl group can be any of those described above. In some embodiments, the number of atoms in the alkyl and heteroaryl portion are designated separately, e.g., C₁₋₆ alkyl-5 to 10 membered heteroaryl having one to four heteroatoms each independently N, O, or S. Alkylheteroaryl groups can be unsubstituted or substituted.

“Oxo” as used herein refers to ═O.

“Substituted” as used herein refers to wherein one or more hydrogen atoms of the group are independently replaced by one or more substituents (e.g., 1, 2, 3, or 4 or more) as indicated.

A “compound of the present application” includes compounds disclosed herein, for example a compound of the present application includes compounds of Formula I-A-1, including the compounds of the Examples. In some embodiments, a “compound of the present application” includes compounds of of Formula I-A-1.

“Pharmaceutically acceptable excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Therapeutically effective amount” or “effective amount” as used herein refers to an amount that is effective to elicit the desired biological or medical response, including the amount of a compound that, when administered to a subject for treating a disease, is sufficient to affect such treatment for the disease. The effective amount will vary depending on the compound, the disease, and its severity and the age, weight, etc., of the subject to be treated. The effective amount can include a range of amounts. As is understood in the art, an effective amount may be in one or more doses, i.e., a single dose or multiple doses may be required to achieve the desired treatment endpoint. An effective amount may be considered in the context of administering one or more therapeutic agents, and a single agent may be considered to be given in an effective amount if, in conjunction with one or more other agents, a desirable or beneficial result may be or is achieved. Suitable doses of any co-administered compounds may optionally be lowered due to the combined action (e.g., additive or synergistic effects) of the compounds.

“Co-administration” as used herein refers to administration of unit dosages of the compounds disclosed herein before or after administration of unit dosages of one or more additional therapeutic agents, for example, administration of the compound disclosed herein within seconds, minutes, or hours of the administration of one or more additional therapeutic agents. For example, in some embodiments, a unit dose of a compound of the present application is administered first, followed within seconds or minutes by administration of a unit dose of one or more additional therapeutic agents. Alternatively, in other embodiments, a unit dose of one or more additional therapeutic agents is administered first, followed by administration of a unit dose of a compound of the present application within seconds or minutes. In some embodiments, a unit dose of a compound of the present application is administered first, followed, after a period of hours (e.g., 1-12 hours), by administration of a unit dose of one or more additional therapeutic agents. In other embodiments, a unit dose of one or more additional therapeutic agents is administered first, followed, after a period of hours (e.g., 1-12 hours), by administration of a unit dose of a compound of the present application. Co-administration of a compound disclosed herein with one or more additional therapeutic agents generally refers to simultaneous or sequential administration of a compound disclosed herein and one or more additional therapeutic agents, such that therapeutically effective amounts of each agent are present in the body of the subject.

Provided are also pharmaceutically acceptable salts, hydrates, solvates, tautomeric forms, polymorphs, and prodrugs of the compounds described herein.

Pharmaceutically acceptable” or “physiologically acceptable” refer to compounds, salts, compositions, dosage forms and other materials which are useful in preparing a pharmaceutical composition that is suitable for veterinary or human pharmaceutical use.

The compounds described herein may be prepared and/or formulated as pharmaceutically acceptable salts or when appropriate as a free base. Pharmaceutically acceptable salts are non-toxic salts of a free base form of a compound that possesses the desired pharmacological activity of the free base. These salts may be derived from inorganic or organic acids or bases. For example, a compound that contains a basic nitrogen may be prepared as a pharmaceutically acceptable salt by contacting the compound with an inorganic or organic acid. Non-limiting examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in Remington: The Science and Practice of Pharmacy, 21st Edition, Lippincott Williams and Wilkins, Philadelphia, Pa., 2006.

Examples of “pharmaceutically acceptable salts” of the compounds disclosed herein also include salts derived from an appropriate base, such as an alkali metal (for example, sodium, potassium), an alkaline earth metal (for example, magnesium), ammonium and N(C₁-C₄ alkyl)₄⁺. Also included are base addition salts, such as sodium or potassium salts.

Provided are also compounds described herein or pharmaceutically acceptable salts, isomers, or a mixture thereof, in which from 1 to n hydrogen atoms attached to a carbon atom may be replaced by a deuterium atom or D, in which n is the number of hydrogen atoms in the molecule. As known in the art, the deuterium atom is a non-radioactive isotope of the hydrogen atom. Such compounds may increase resistance to metabolism, and thus may be useful for increasing the half-life of the compounds described herein or pharmaceutically acceptable salts, isomer, or a mixture thereof when administered to a mammal. See, e.g., Foster, “Deuterium Isotope Effects in Studies of Drug Metabolism”, Trends Pharmacol. Sci., 5(12):524-527 (1984). Such compounds are synthesized by means well known in the art, for example by employing starting materials in which one or more hydrogen atoms have been replaced by deuterium.

Examples of isotopes that can be incorporated into the disclosed compounds also include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I and ¹²⁵I respectively. Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of Formula I-A-1 can generally be prepared by conventional techniques known to those skilled in the art or by processes analogous to those described in the Examples as set out below using an appropriate isotopically-labeled reagent in place of the non-labeled reagent previously employed.

The compounds of the embodiments disclosed herein, or their pharmaceutically acceptable salts may contain one or more asymmetric centers and may thus give rise to enantiomers, tautomer, diastereomers, and other stereoisomeric forms that may be defined, in terms of absolute stereochemistry, as (R)- or (S)- or, as (D)- or (L)- for amino acids, as well as deuterated analogs thereof. The chemical formula shown in the present application is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included. Where compounds are represented in their chiral form, it is understood that the embodiment encompasses, but is not limited to, the specific diastereomerically or enantiomerically enriched form. Where chirality is not specified but is present, it is understood that the embodiment is directed to either the specific diastereomerically or enantiomerically enriched form; or a racemic or scalemic mixture of such compound(s). As used herein, “scalemic mixture” is a mixture of stereoisomers at a ratio other than 1:1

“Stereoisomer” as used herein refers to a compound made up of the same atoms bonded by the same bonds but having different three-dimensional structures, which are not interchangeable. The present application contemplates various stereoisomers and mixtures thereof and includes “enantiomers”, which refers to two stereoisomers whose molecules are non-superimposable mirror images of one another.

“Tautomer” as used herein refers to a proton shift from one atom of a molecule to another atom of the same molecule. In some embodiments, the present application includes tautomers of said compounds.

“Solvate” as used herein refers to the result of the interaction of a solvent and a compound. Solvates of salts of the compounds described herein are also provided. Hydrates of the compounds described herein are also provided.

“Hydrate” as used herein refers to a compound of the disclosure that is chemically associated with one or more molecules of water.

“Prevention” or “preventing” means any treatment of a disease or condition that causes the clinical symptoms of the disease or condition not to develop. Compounds may, in some embodiments, be administered to a subject (including a human) who is at risk or has a family history of the disease or condition.

“Prodrug” as used herein refers to a derivative of a drug that upon administration to the human body is converted to the parent drug according to some chemical or enzymatic pathway. In some embodiments, a prodrug is a biologically inactive derivative of a drug that upon administration to the human body is converted to the biologically active parent drug according to some chemical or enzymatic pathway.

“Treatment” or “treat” or “treating” as used herein refers to an approach for obtaining beneficial or desired results. For purposes of the present application, beneficial or desired results include, but are not limited to, alleviation of a symptom and/or diminishment of the extent of a symptom and/or preventing a worsening of a symptom associated with a disease or condition. In one embodiment, “treatment” or “treating” includes one or more of the following: a) inhibiting the disease or condition (e.g., decreasing one or more symptoms resulting from the disease or condition, and/or diminishing the extent of the disease or condition); b) slowing or arresting the development of one or more symptoms associated with the disease or condition (e.g., stabilizing the disease or condition, delaying the worsening or progression of the disease or condition); and c) relieving the disease or condition, e.g., causing the regression of clinical symptoms, ameliorating the disease state, delaying the progression of the disease, increasing the quality of life, and/or prolonging survival. “At risk individual” as used herein refers to an individual who is at risk of developing a condition to be treated. An individual “at risk” may or may not have detectable disease or condition, and may or may not have displayed detectable disease prior to the treatment of methods described herein. “At risk” denotes that an individual has one or more so-called risk factors, which are measurable parameters that correlate with development of a disease or condition and are known in the art. An individual having one or more of these risk factors has a higher probability of developing the disease or condition than an individual without these risk factor(s).

### II. Compounds

One aspect of the present application relates to compounds that binds to and act as agonist or modulators of GLP-1R. In some embodiments, the present application provides a compound of Formula I-A-1:

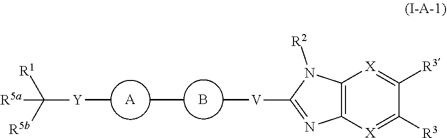

or a pharmaceutically acceptable salt thereof, wherein
R¹ is C₁₋₆ alkyl, C₁₋₆ haloalkyl, C₂₋₆ alkenyl, C₂₋₆ alkynyl, C₃₋₁₀ cycloalkyl, heterocyclyl, C₆₋₁₀ aryl, 5-10 membered heteroaryl, —S—R^{1b}, —S(O)R^{1b}, —S(O)(NH)R^{1b}, —S(O)₂R^{1b}, —S(O)₂N(R^{1b})(R^{1c}), —S(O)(NR^{1b})R^{1c}, —C(O)N(R^{1b})(R^{1c}), —C(O)R^{1b}, or —C(O)OR^{1c}, wherein the alkyl, haloalkyl, cycloalkyl, aryl, 5-10 membered heteroaryl, or heterocyclyl is each optionally substituted with one to four Z¹ and wherein any two Z¹ can be combined with the atoms to which they are attached to form a C₃₋₁₀ cycloalkyl or heterocyclyl or C₆₋₁₀ aryl or 5-10 membered heteroaryl;
Ring A is C₃₋₁₀ cycloalkyl, heterocyclyl, C₆₋₁₀ aryl or 5-10 membered heteroaryl, which is each optionally substituted with one to four Z^{1a};
Ring B is C₃₋₁₀ cycloalkyl, heterocyclyl, C₆₋₁₀ aryl or 5-10 membered heteroaryl, which is each optionally substituted with one to four R⁴;
Ring A and Ring B each contains zero to three double bonds;
R² is H, C₁₋₆ alkyl, —C₁₋₆ alkyl-alkoxy, —C₁₋₆ alkyl-cycloalkoxy, C₁₋₆ haloalkyl, C₂₋₆ alkenyl, C₂₋₆ alkynyl, C₃₋₁₀ cycloalkyl, heterocyclyl, C₆₋₁₀ aryl, 5-10 membered heteroaryl, —CN, —OR^{2a}, —S—R^{2a}, —S(O)R^{2a}, —S(O)(NH)R^{2a}, —S(O)₂R^{2a}, —S(O)₂N(R^{2a})(R^{2b}), or —S(O)(NR^{2a})R^{2b}, wherein the alkyl, alkoxy, cycloalkoxy, haloalkyl, cycloalkyl, heterocyclyl, aryl, or 5-10 membered heteroaryl is each optionally substituted with one to four Z¹ and wherein any two Z₁ can be combined with the atoms to which they are attached to form a C₃₋₁₀ cycloalkyl or heterocyclyl or C₆₋₁₀ aryl or 5-10 membered heteroaryl;
X is each independently —N═, —C(H)═, or —C(R⁸)═;
each R³ and R^{3′} is independently H, C₁₋₆ alkyl, C₁₋₆ haloalkyl, C₂₋₆ alkenyl, C₂₋₆ alkynyl, halogen, C₃₋₁₀ cycloalkyl, heterocyclyl, C₆₋₁₀ aryl, 5-10 membered heteroaryl, —CN, —NO₂, —OR^{3a}, —C(O)R^{3a}, —CH₂C(O)OR^{3a}, —C(O)OR^{3a}, —C(O)N(R^{3a})(R^{3b}), —N(R^{3a})C(O)R^{3b}, —N(R^{3a})C(O)OR^{3b}, —N(R^{3a})C(O)N(R^{3b})₂, —C(O)NHS(O)₂R^{3a}, —C(O)NR^{3a}S(O)₂R^{3b}, —C(O)NR^{3a}S(O)₂NR^{3b}R^{3c}, —C(O)NR^{3a}—S(O)(═NR^{3b})R^{3c}, —S(O)₂R^{3a}, —S(O)₂OR^{3a}, —S(O)₂N(R^{3a})(R^{3b}), —N(R^{3a})S(O)₂R^{3b}, —S(O)₂NHC(O)R^{3a}, —S(O)(═NR^{3a})R^{3b}, —S(O)(═NR^{3a})NR^{3b}, —S(═NR^{3a})(═NR^{3b})R^{3c}, —P(O)(OR^{3a})(R^{3b}), —P(O)(OR^{3a})(OR^{3b}), —B(OR^{3a})(OR^{3b}), or —O—C₁₋₆alkyl-C(O)OR^{3a}, wherein the alkyl, haloalkyl, alkenyl, alkynyl, cycloalkyl, heterocyclyl, aryl, or 5-10 membered heteroaryl in each instance is optionally substituted with one to four R^{3d};
each R^{3a}, R^{3b}, and R^{3c} is independently H, C₁₋₆ alkyl, C₆₋₁₀ aryl, 5-10 membered heteroaryl, C₁₋₆ haloalkyl, C₂₋₈ alkoxyalkyl, —C₁₋₄ alkyl-N(R^{9a})(R^{9b}), —C₁₋₄ alkyl-N(R^{9a})C(O)—O—C₁₋₄ alkyl-OP(O)(OR^{9c})₂, —C₁₋₄ alkyl-C(O)N(R^{9a})(R^{9b}), —C₁₋₄ alkyl-O—C(O)—O—C₁₋₄ alkyl, —C₁₋₄ alkyl-O—C(O)—C₁₋₄ alkyl-N(R^{9a})(R^{9b}), —C₁₋₄ alkyl-O—C(O)—C₁₋₄ alkyl-OP(O)(OR^{9c})₂, —C₁₋₄ alkyl-C₃₋₈ cycloalkyl, —C₁₋₄ alkyl-heterocyclyl, C₂₋₆ alkenyl, C₂₋₆ alkynyl, C₃₋₁₀ cycloalkyl, heterocyclyl, —CH₂CH(N(R^{9a})₂)C(O)OR^{9b}, —P(O)(OR^{9c})₂, —OP(O)(OR^{9c})₂, —CH₂P(O)(OR^{9c})₂, —CH₂OP(O)(OR^{9c})₂, —OCH₂P(O)(OR^{9c})₂, —C(O)OCH₂P(O)(OR^{9c})₂, —P(O)(R^{9c})(OR^{9d}), —OP(O)(R^{9c})(OR^{9d}), —CH₂P(O)(R^{9c})(OR^{9d}), —OCH₂P(O)(R^{9c})(OR^{9d}), —C(O)OCH₂P(O)(R^{9c})(OR^{9d}), —P(O)(N(R^{9c})₂)₂, —OP(O)(N(R^{9c})₂)₂, —CH₂P(O)(N(R^{9c})₂)₂, —OCH₂P(O)(N(R^{9c})₂)₂, —C(O)OCH₂P(O)(N(R^{9c})₂)₂, —P(O)(N(R^{9c})₂)(OR^{9d}), —OP(O)(N(R^{9c})₂)(OR^{9d}), —CH₂P(O)(N(R^{9c})₂)(OR^{9d}), —OCH₂P(O)(N(R^{9c})₂)(OR^{9d}), —C(O)OCH₂P(O)(N(R^{9c})₂)(OR^{9d}), —P(O)(R^{9c})(N(R^{9d})₂), —OP(O)(R^{9c})(N(R^{9d})₂), —CH₂P(O)(R^{9c})(N(R^{9d})₂), —OCH₂P(O)(R^{9c})(N(R^{9d})₂), —C(O)OCH₂P(O)(R^{9c})(N(R^{9d})₂), C₁₋₆ alkyl-heterocyclyl, C₁₋₆ alkyl-aryl or C₁₋₆ alkyl-(5-10 membered) heteroaryl, wherein the alkyl, alkenyl, cycloalkyl, heterocyclyl, aryl, or 5-10 membered heteroaryl in each instance is optionally substituted with one to four Z^{1b};
each R⁴ is independently H, —OH, CH₂OH, C₁₋₉ alkyl, C₁₋₈ haloalkyl, C₁₋₆ haloalkoxy, C₂₋₆ alkoxyalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —NO₂, —CN, —N₃, —O—R^{4a}, —C(O)R^{4a}, —C(O)O—R^{4a}, —C(O)N(R^{4a})(R^{4b}), —N(R^{4a})(R^{4b}), —N(R^{4a})₂(R^{4b})⁺, —N(R^{4a})—C(O)R^{4b}, —N(R^{4a})C(O)O(R^{4b}), —N(R^{4a})C(O)N(R^{4b})(R^{4c}), —N(R^{4a})S(O)₂(R^{4b}), —N(R^{4a})S(O)₂—N(R^{4b})(R^{4c}), —N(R^{4a})S(O)₂O(R^{4b}), —OC(O)R^{4a}, —OC(O)OR^{4a}, —OC(O)—N(R^{4a})(R^{4b}), —S—R^{4a}, —S(O)R^{4a}, —S(O)(NH)R^{4a}, —S(O)₂R^{4a}, —S(O)₂N(R^{4a})(R^{4b}), —S(O)(NR^{4a})R^{4b}, or —Si(R^{4a})₃, wherein the alkyl, haloalkyl, alkenyl, alkynyl, cycloalkyl, heterocyclyl, aryl, or 5-10 membered heteroaryl in each instance is optionally substituted with one to four Z^{1b}; or two R⁴ groups attached to adjacent ring atoms are combined with the atoms to which they are attached to form a C₅₋₁₀ cycloalkyl or heterocyclyl, which is each optionally substituted with one to four Z^{1b};
each R^{5a} and R^{5b} is independently H, C₁₋₆ alkyl, C₁₋₆ alkyloxy, C₁₋₆ haloalkyl, C₂₋₆ alkoxyalkyl, halogen, C₃₋₁₀ cycloalkyl, heterocyclyl, —C₁₋₆ alkyl-N(R^{9a})(R^{9b}), —CN, —OR^{5a1}, or —N(R^{5a1})(R^{5a2}); or R^{5a} and R^{5b} are combined with the atoms to which they are attached to form a C₃₋₁₀ cycloalkyl or heterocyclyl, which is each optionally substituted with one to four R^{5a3}; or R^{5a} or R^{5b} can be combined with the substituents on Ring A to form a C₅₋₁₀ cycloalkyl or heterocyclyl, which is each optionally substituted with one to four R^{5a3};
each R^{5a1} and R^{5a2} is independently H, C₁₋₆ alkyl, C₂₋₆ alkenyl, C₂₋₆ alkynyl, C₃₋₁₀ cycloalkyl, heterocyclyl, C₆₋₁₀ aryl, or 5-10 membered heteroaryl, wherein the cycloalkyl, heterocyclyl, aryl, or 5-10 membered heteroaryl is each optionally substituted with one to four R^{5a4};
V is —C(O)—, —O—, —N(R^{6a})—, or —C(R^{6b})(R^{6c})—;
R^{6a} is H, C₁₋₆ alkyl, C₃₋₁₀ cycloalkyl, heterocyclyl, —S(O)₂R^{6a1}, or —S(O)₂N(R^{6a1})(R^{6a2}), wherein the cycloalkyl or heterocyclyl in each instance is optionally substituted with C₁₋₆ alkyl, F, or —CN;
each R^{6b} and R^{6c} is independently H, C₁₋₆ alkyl, C₁₋₆ haloalkyl, C₂₋₆ alkoxyalkyl, halogen, C₃₋₁₀ cycloalkyl, heterocyclyl, —C₁₋₆ alkyl-N(R^{9a})(R^{9b}), —CN, —OR^{6c1}, or —N(R^{6c2})(R^{6c3}), wherein the alkyl, cycloalkyl, or heterocyclyl in each instance is optionally substituted with one to four R^{6b1}; or R^{6b} and R^{6c} are combined with the atom to which they are attached to form C₃₋₁₀ cycloalkyl or heterocyclyl, which is each optionally substituted with one to four R^{6b1}; or R^{6a} or R^{6c} is combined with one R⁴ group and the atoms to which they are attached to form a C₅₋₁₀ cycloalkyl or heterocyclyl, which is each optionally substituted with one to four R¹⁰;
Y is —N(R⁷)—, —O—, —S—, —S(O)—, —S(O)₂—, —S(O)(═NH)—, or —S(O)(═NR⁷)—;
each R^{3d}, R^{5a3}, R^{5a4}, R^{6b1}, and R¹⁰ is independently H, C₁₋₆ alkyl, C₁₋₆ haloalkyl, C₁₋₆ alkoxy, C₁₋₆ haloalkoxy, C₂₋₆ alkoxyalkyl, C₂₋₆ alkenyl, C₂₋₆ alkynyl, halogen, C₃₋₁₀ cycloalkyl, heterocyclyl, C₆₋₁₀ aryl, 5-10 membered heteroaryl, oxo, —OH, —CN, CO₂R^{3e}, —NO₂, or —C(O)N(R^{2a})(R^{2b}), wherein the heterocyclyl or 5-10 membered heteroaryl in each instance is optionally substituted with C₁₋₆ alkyl, C₁₋₆ haloalkyl, or C₁₋₆ haloalkoxy;
each R^{6a1}, R^{6a2}, R^{6c1}, R^{6c2}, R^{6c3} and R⁷ is independently H, C₁₋₆ alkyl or C₃₋₁₀ cycloalkyl;
each R⁸ is independently C₁₋₉ alkyl, C₁₋₈ haloalkyl, C₂₋₆ alkenyl, C₂₋₆ alkynyl, halogen, oxo, —OH, —CN, CO₂R^{3e}, —NO₂, —NH₂, —N₃, —SH, —O(C₁₋₉ alkyl), —O(C₁₋₈ haloalkyl), —NH(C₁₋₉ alkyl), —NH(C₁₋₈ haloalkyl), —N(C₁₋₉ alkyl)₂, —N(C₁₋₈ haloalkyl)₂;
each R^{9a} and R^{9b} is independently H, C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —NO₂, —CN, —N₃, —O—R^{12a}, —C(O)R^{12a}, —C(O)O—R^{12a}, —C(O)N(R^{12a})(R^{12b}), —N(R^{12a})(R^{12b}), —N(R^{12a})₂(R^{12b})⁺, —N(R^{12a})—C(O)R^{12b}, —N(R^{12a})C(O)O(R^{12b}), —N(R^{12a})C(O)N(R^{12b})(R^{12c}), —N(R^{12a})S(O)₂(R^{12b}), —N(R^{12a})S(O)₂—N(R^{12b})(R^{12c}), —N(R^{12a})S(O)₂O(R^{12b}), —OC(O)R^{12a}, —OC(O)OR^{12a}, —OC(O)—N(R^{12a})(R^{12b}), —S—R^{12a}, —S(O)R^{12a}, —S(O)(NH)R^{12a}, —S(O)₂R^{12a}, —S(O)₂N(R^{12a})(R^{12b}), —S(O)(NR^{12a})R^{12b}, or —Si(R^{12a})₃; or R^{9a} and R^{9b} together form a 3-10 membered heterocyclyl optionally substituted with C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —NO₂, —CN, —N₃, —O—R^{12a}, —C(O)R^{12a}, —C(O)O—R^{12a}, —C(O)N(R^{12a})(R^{12b}), —N(R^{12a})(R^{12b}), —N(R^{12a})₂(R^{12b})⁺, —N(R^{12a})—C(O)R^{12b}, —N(R^{12a})C(O)O(R^{12b}), —N(R^{12a})C(O)N(R^{12b})(R^{12c}), —N(R^{12a})S(O)₂(R^{12b}), —N(R^{12a})S(O)₂—N(R^{12b})(R^{12c}), —N(R^{12a})S(O)₂O(R^{12b}), —OC(O)R^{12a}, —OC(O)OR^{12a}, —OC(O)—N(R^{12a})(R^{12b}), —S—R^{12a}, —S(O)R^{12a}, —S(O)(NH)R^{12a}, —S(O)₂R^{12a}, —S(O)₂N(R^{12a})(R^{12b}), —S(O)(NR^{12a})R^{12b}, or —Si(R^{12a})₃;
each Z¹ is independently C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —NO₂, —N₃, —CN, —O—R^{12a}, —C(O)—R^{12a}, —C(O)O—R^{12a}, —C(O)—N(R^{12a})(R^{12b}), —N(R^{12a})(R^{12b}), —N(R^{12a})₂(R^{12b})⁺, —N(R^{12a})C(O)—R^{12b}, —N(R^{12a})C(O)O—R^{12b}, —N(R^{12a})C(O)N(R^{12b})(R^{12c}), —N(R^{12a})S(O)₂(R^{12b}), —NR^{12a}S(O)₂N(R^{12b})(R^{12c}), —NR^{12a}S(O)₂O(R^{12b}), —OC(O)R^{12a}, —OC(O)OR^{12a}, —OC(O)—N(R^{12a})(R^{12b}), —S—R^{12a}, —S(O)R^{12a}, —S(O)(NH)R^{12a}, —S(O)₂R^{12a}, —S(O)₂N(R^{12a})(R^{12b}), —S(O)(NR^{12a})R^{12b}, or —Si(R^{12a})₃, wherein the alkyl, haloalkyl, alkenyl, alkynyl, cycloalkyl, heterocyclyl, aryl, or 5-10 membered heteroaryl in each instance is optionally substituted with one to four Z^{1a};
each Z^{1a} is independently H, C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —NO₂, —CN, —N₃, —O—R^{12a}, —C(O)R^{12a}, —C(O)O—R^{12a}, —C(O)N(R^{12a})(R^{12b}), —N(R^{12a})(R^{12b}), —N(R^{12a})₂(R^{12b})⁺, —N(R^{12a})—C(O)R^{12b}, —N(R^{12a})C(O)O(R^{12b}), —N(R^{12a})C(O)N(R^{12b})(R^{12c}), —N(R^{12a})S(O)₂(R^{12b}), —N(R^{12a})S(O)₂—N(R^{12b})(R^{12c}), —N(R^{12a})S(O)₂O(R^{12b}), —OC(O)R^{12a}, —OC(O)OR^{12a}, —OC(O)—N(R^{12a})(R^{12b}), —S—R^{12a}, —S(O)R^{12a}, —S(O)(NH)R^{12a}, —S(O)₂R^{12a}, —S(O)₂N(R^{12a})(R^{12b}), —S(O)(NR^{12a})R^{12b}, or —Si(R^{12a})₃; wherein the alkyl, haloalkyl, alkenyl, alkynyl, cycloalkyl, heterocyclyl, aryl, or 5-10 membered heteroaryl in each instance is optionally substituted with one to four Z^{1b};
each Z^{1b} is independently H, C₁₋₉ alkyl, C₁₋₈ haloalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —OH, —CN, CO₂R^{3e}, —NO₂, —NH₂, —N₃, —SH, —O(C₁₋₉ alkyl), —O(C₁₋₈ haloalkyl), —O(C₂₋₆ alkenyl), —O(C₂₋₆ alkynyl), —O(C₃₋₁₅ cycloalkyl), —O(heterocyclyl), —O(C₆₋₁₀ aryl), —O(5-10 membered heteroaryl), —NH(C₁₋₉ alkyl), —NH(C₁₋₈ haloalkyl), —NH(C₂₋₆ alkenyl), —NH(C₂₋₆ alkynyl), —NH(C₃₋₁₅ cycloalkyl), —NH(heterocyclyl), —NH(C₆₋₁₀ aryl), —NH(5-10 membered heteroaryl), —N(cyclo-alkyl), —N(C₁₋₉ alkyl)₂, —N(C₁₋₈ haloalkyl)₂, —N(C₂₋₆ alkenyl)₂, —N(C₂₋₆ alkynyl)₂, —N(C₃₋₁₅ cycloalkyl)₂, —N(heterocyclyl)₂, —N(C₆₋₁₀ aryl)₂, —N(5-10 membered heteroaryl)₂, —N(C₁₋₉ alkyl)(C₁₋₈ haloalkyl), —N(C₁₋₉ alkyl)(C₂₋₆ alkenyl), —N(C₁₋₉ alkyl)(C₂₋₆ alkynyl), —N(C₁₋₉ alkyl)(C₃₋₁₅ cycloalkyl), —N(C₁₋₉ alkyl)(heterocyclyl), —N(C₁₋₉ alkyl)(C₆₋₁₀ aryl), —N(C₁₋₉ alkyl)(5-10 membered heteroaryl), —C(O)(C₁₋₉ alkyl), —C(O)(C₁₋₈ haloalkyl), —C(O)(C₂₋₆ alkenyl), —C(O)(C₂₋₆ alkynyl), —C(O)(C₃₋₁₅ cycloalkyl), —C(O)(heterocyclyl), —C(O)(C₆₋₁₀ aryl), —C(O)(5-10 membered heteroaryl), —C(O)O(C₁₋₉ alkyl), —C(O)O(C₁₋₈ haloalkyl), —C(O)O(C₂₋₆ alkenyl), —C(O)O(C₂₋₆ alkynyl), —C(O)O(C₃₋₁₅ cycloalkyl), —C(O)O(heterocyclyl), —C(O)O(C₆₋₁₀ aryl), —C(O)O(5-10 membered heteroaryl), —C(O)NH₂, —C(O)NH(C₁₋₉ alkyl), —C(O)NH(C₁₋₈ haloalkyl), —C(O)NH(C₂₋₆ alkenyl), —C(O)NH(C₂₋₆ alkynyl), —C(O)NH(C₃₋₁₅ cycloalkyl), —C(O)NH(heterocyclyl), —C(O)NH(C₆₋₁₀ aryl), —C(O)NH(5-10 membered heteroaryl), —C(O)N(C₁₋₉ alkyl)₂, —C(O)N(C₁₋₈ haloalkyl)₂, —C(O)N(C₂₋₆ alkenyl)₂, —C(O)N(C₂₋₆ alkynyl)₂, —C(O)N(C₃₋₁₅ cycloalkyl)₂, —C(O)N(heterocyclyl)₂, —C(O)N(C₆₋₁₀ aryl)₂, —C(O)N(5-10 membered heteroaryl)₂, —NHC(O)(C₁₋₉ alkyl), —NHC(O)(C₁₋₈ haloalkyl), —NHC(O)(C₂₋₆ alkenyl), —NHC(O)(C₂₋₆ alkynyl), —NHC(O)(C₃₋₁₅ cycloalkyl), —NHC(O)(heterocyclyl), —NHC(O)(C₆₋₁₀ aryl), —NHC(O)(5-10 membered heteroaryl), —NHC(O)O(C₁₋₉ alkyl), —NHC(O)O(C₁₋₈ haloalkyl), —NHC(O)O(C₂₋₆ alkenyl), —NHC(O)O(C₂₋₆ alkynyl), —NHC(O)O(C₃₋₁₅ cycloalkyl), —NHC(O)O(heterocyclyl), —NHC(O)O(C₆₋₁₀ aryl), —NHC(O)O(5-10 membered heteroaryl), —NHC(O)NH(C₁₋₉ alkyl), —NHC(O)NH(C₁₋₈ haloalkyl), —NHC(O)NH(C₂₋₆ alkenyl), —NHC(O)NH(C₂₋₆ alkynyl), —NHC(O)NH(C₃₋₁₅ cycloalkyl), —NHC(O)NH(heterocyclyl), —NHC(O)NH(C₆₋₁₀ aryl), —NHC(O)NH(5-10 membered heteroaryl), —NHS(O)(C₁₋₉ alkyl), —N(C₁₋₉ alkyl)(S(O)(C₁₋₉ alkyl), —S(C₁₋₉ alkyl), —S(C₁₋₈ haloalkyl), —S(C₂₋₆ alkenyl), —S(C₂₋₆ alkynyl), —S(C₃₋₁₅ cycloalkyl), —S(heterocyclyl), —S(C₆₋₁₀ aryl), —S(5-10 membered heteroaryl), —S(O)N(C₁₋₉ alkyl)₂, —S(O)(C₁₋₉ alkyl), —S(O)(C₁₋₈ haloalkyl), —S(O)(C₂₋₆ alkenyl), —S(O)(C₂₋₆ alkynyl), —S(O)(C₃₋₁₅ cycloalkyl), —S(O)(heterocyclyl), —S(O)(C₆₋₁₀ aryl), —S(O)(5-10 membered heteroaryl), —S(O)₂(C₁₋₉ alkyl), —S(O)₂(C₁₋₈ haloalkyl), —S(O)₂(C₂₋₆ alkenyl), —S(O)₂(C₂₋₆ alkynyl), —S(O)₂(C₃₋₁₅ cycloalkyl), —S(O)₂(heterocyclyl), —S(O)₂(C₆₋₁₀ aryl), —S(O)₂(5-10 membered heteroaryl), —S(O)(NH)(C₁₋₉ alkyl), —S(O)₂NH(C₁₋₉ alkyl), or —S(O)₂N(C₁₋₉ alkyl)₂, wherein the alkyl, cycloalkyl, heterocyclyl, aryl, or 5-10 membered heteroaryl in each instance is optionally substituted with one to three C₁₋₉ alkyl, C₁₋₈ haloalkyl, halogen, —OH, —NH₂, CO₂H, —O(C₁₋₉ alkyl), —O(C₁₋₈ haloalkyl), —O(C₃₋₁₅ cycloalkyl), —O(heterocyclyl), —O(aryl), —O(5-10 membered heteroaryl), —NH(C₁₋₉ alkyl), —NH(C₁₋₈ haloalkyl), —NH(C₃₋₁₅ cycloalkyl), —NH(heterocyclyl), —NH(aryl), —NH(5-10 membered heteroaryl), —N(C₁₋₉ alkyl)₂, —N(C₃₋₁₅ cycloalkyl)₂, —NHC(O)(C₁₋₈ haloalkyl), —NHC(O)(C₃₋₁₅ cycloalkyl), —NHC(O)(heterocyclyl), —NHC(O)(aryl), —NHC(O)(5-10 membered heteroaryl), —NHC(O)O(C₁₋₉ alkyl), —NHC(O)O(C₁₋₈ haloalkyl), —NHC(O)O(C₂₋₆ alkynyl), —NHC(O)O(C₃₋₁₅ cycloalkyl), —NHC(O)O(heterocyclyl), —NHC(O)O(aryl), —NHC(O)O(5-10 membered heteroaryl), —NHC(O)NH(C₁₋₉ alkyl), S(O)₂(C₁₋₉ alkyl), —S(O)₂(C₁₋₈ haloalkyl), —S(O)₂(C₃₋₁₅ cycloalkyl), —S(O)₂(heterocyclyl), —S(O)₂(aryl), —S(O)₂(5-10 membered heteroaryl), —S(O)(NH)(C₁₋₉ alkyl), —S(O)₂NH(C₁₋₉ alkyl), or —S(O)₂N(C₁₋₉ alkyl)₂;
each R^{1b}, R^{1c}, R^{2a}, R^{2b}, R^{4a}, R^{4b}, R^{4c}, R^{9c}, R^{9d}, R^{12a}, R^{12b}, and R^{12c} is independently H, —OH, C₁₋₉ alkyl, C₂₋₆ alkenyl, C₂₋₆ alkynyl, C₃₋₁₅ cycloalkyl, heterocyclyl, C₆₋₁₀ aryl, or 5-10 membered heteroaryl wherein the alkyl, alkenyl, alkynyl, cycloalkyl, heterocyclyl, aryl, or 5-10 membered heteroaryl is each optionally substituted with one to four Z^{1b}; and
each R^{3e} is independently H, C₁₋₆ alkyl, C₁₋₆ haloalkyl, —C₁₋₄ alkyl-N(R^{9a})(R^{9b}), —C₁₋₄ alkyl-C(O)N(R^{9a})(R^{9b}), —C₁₋₄ alkyl-O—C(O)—O—C₁₋₄ alkyl, —C₁₋₄ alkyl-O—C(O)—C₁₋₄ alkyl-N(R^{9a})(R^{9b}), —C₁₋₄ alkyl-C₃₋₈ cycloalkyl, —C₁₋₄ alkyl-heterocyclyl, C₃₋₁₀ cycloalkyl, heterocyclyl, C₆₋₁₀ aryl, 5-10 membered heteroaryl, —P(O)(OR^{9c})₂, —CH₂P(O)(OR^{9c})₂, —OCH₂P(O)(OR^{9c})₂, —C(O)OCH₂P(O)(OR^{9c})₂, —P(O)(R^{9c})(OR^{9d}), —OP(O)(R^{9c})(OR^{9d}), —CH₂P(O)(R^{9c})(OR^{9d}), —C(O)OCH₂P(O)(R^{9c})(OR^{9d}), —P(O)(N(R^{9c})₂)₂, —CH₂P(O)(N(R^{9c})₂)₂, —C(O)OCH₂P(O)(N(R^{9c})₂)₂, —P(O)(N(R^{9c})₂)(OR^{9d}), —CH₂P(O)(N(R^{9c})₂)(OR^{9d}), —C(O)OCH₂P(O)(N(R^{9c})₂)(OR^{9d}), —P(O)(R^{9c})(N(R^{9d})₂), —CH₂P(O)(R^{9c})(N(R^{9d})₂), or —C(O)OCH₂P(O)(R^{9c})(N(R^{9d})₂), wherein the alkyl, alkenyl, cycloalkyl, heterocyclyl, aryl, or 5-10 membered heteroaryl in each instance is optionally substituted with one to four Z^{1b}, wherein each 5-10 membered heteroaryl has 5 to 12 ring members and has one to four heteroatoms, each is independently N, O, or S, and wherein each heterocyclyl has 3 to 12 ring members and has one to four heteroatoms, each is independently N, O, or S.

In some embodiments, each Z¹ in Formula I-A-1, or a pharmaceutically acceptable salt thereof, is independently C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —NO₂, —N₃, —CN, —O—R^{12a}, —C(O)—R^{12a}, —C(O)O—R^{12a}, —C(O)—N(R^{12a})(R^{12b}), or —N(R^{12a})(R^{12b}).

In some embodiments, each Z^{1a} in Formula I-A-1, or a pharmaceutically acceptable salt thereof, is independently H, —OH, C₁₋₉ alkyl, C₁₋₈ haloalkyl, C₁₋₆ alkoxy, C₁₋₆ haloalkoxy, C₂₋₆ alkoxyalkyl, or halogen.

In some embodiments, each Z^{1b} in Formula I-A-1, or a pharmaceutically acceptable salt thereof, is independently H, C₁₋₉ alkyl, C₁₋₈ haloalkyl, halogen, oxo, —OH, or —CN.

In some embodiments, each R⁸ in Formula I-A-1, or a pharmaceutically acceptable salt thereof, is independently H, oxo, OH, C₁₋₉ alkyl, C₁₋₈ haloalkyl, halogen, —O(C₁₋₃ alkyl), or —O(C₁₋₃ haloalkyl).

Another embodiment concerns compounds of Formula I-A-2 or Formula I-A-3

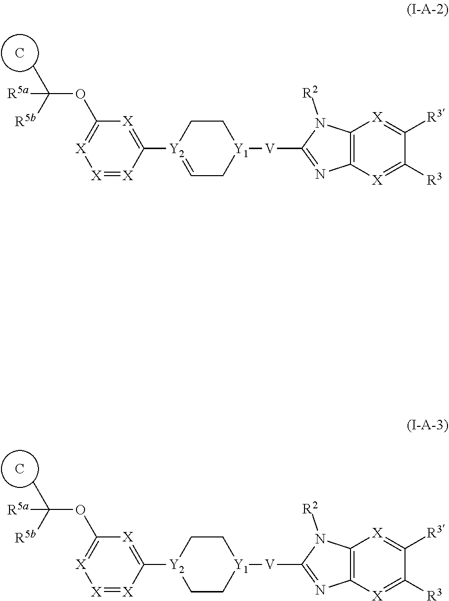

or a pharmaceutically acceptable salt thereof, wherein
Ring C is C₃₋₁₀ cycloalkyl, heterocyclyl, C₆₋₁₀ aryl or 5-10 membered heteroaryl, which is each optionally substituted with one to four Z¹;
Y₁—V is N—CH₂, N—CH(CH₃), N—CF₂, CH—CH₂, CH—CF₂, CH—NH, N—S(O), N—S(O)₂, N—C(O), CH—C(O) or cyclopropyl;
Y₂ is C, CH or N;
R² is H, C₁₋₆ alkyl, —C₁₋₆ alkyl-alkoxy, —C₁₋₆ alkyl-cycloalkoxy, C₁₋₆ haloalkyl, C₂₋₆ alkenyl, C₂₋₆ alkynyl, C₃₋₁₀ cycloalkyl, heterocyclyl, C₆₋₁₀ aryl, 5-10 membered heteroaryl, —CN, —OR^{2a}, —S—R^{2a}, —S(O)R^{2a}, —S(O)(NH)R^{2a}, —S(O)₂R^{2a}, —S(O)₂N(R^{2a})(R^{2b}), or —S(O)(NR^{2a})R^{2b}, wherein the alkyl, alkoxy, cycloalkoxy, haloalkyl, cycloalkyl, heterocyclyl, aryl, or 5-10 membered heteroaryl in each instance is optionally substituted with one to four Z¹, wherein any two Z₁ can be combined with the atoms to which they are attached to form a C₃₋₁₀ cycloalkyl or heterocyclyl or C₆₋₁₀ aryl or 5-10 membered heteroaryl;
X is each independently —N═, —C(H)═, or —C(R⁸)═;
each R³ and R^{3′} is independently H, C₁₋₆ alkyl, C₁₋₆ haloalkyl, C₂₋₆ alkenyl, C₂₋₆ alkynyl, halogen, C₃₋₁₀ cycloalkyl, heterocyclyl, C₆₋₁₀ aryl, 5-10 membered heteroaryl, —CN, —NO₂, —OR^{3a}, —C(O)R^{3a}, —CH₂C(O)OR^{3a}, —C(O)OR^{3a}, —C(O)N(R^{3a})(R^{3b}), —N(R^{3a})C(O)R^{3b}, —N(R^{3a})C(O)OR^{3b}, —N(R^{3a})C(O)N(R^{3b})₂, —C(O)NHS(O)₂R^{3a}, —C(O)NR^{3a}S(O)₂R^{3b}, —C(O)NR^{3a}S(O)₂NR^{3b}R^{3c}, —C(O)NR^{3a}—S(O)(═NR^{3b})R^{3c}, —S(O)₂R^{3a}, —S(O)₂OR^{3a}, —S(O)₂N(R^{3a})(R^{3b}), —N(R^{3a})S(O)₂R^{3b}, —S(O)₂NHC(O)R^{3a}, —S(O)(═NR^{3a})R^{3b}, —S(O)(═NR^{3a})NR^{3b}, —S(═NR^{3a})(═NR^{3b})R^{3c}, —P(O)(OR^{3a})(R^{3b}), —P(O)(OR^{3a})(OR^{3b}), —B(OR^{3a})(OR^{3b}), or —O—C₁₋₆alkyl-C(O)OR^{3a}, wherein the alkyl, haloalkyl, alkenyl, alkynyl, cycloalkyl, heterocyclyl, aryl, or 5-10 membered heteroaryl in each instance is optionally substituted with one to four R^{3d};
each R^{3a}, R^{3b}, and R^{3c} is independently H, C₁₋₆ alkyl, C₆₋₁₀ aryl, 5-10 membered heteroaryl, C₁₋₆ haloalkyl, C₂₋₈ alkoxyalkyl, —C₁₋₄ alkyl-N(R^{9a})(R^{9b}), —C₁₋₄ alkyl-N(R^{9a})C(O)—O—C₁₋₄ alkyl-OP(O)(OR^{9c})₂, —C₁₋₄ alkyl-C(O)N(R^{9a})(R^{9b}), —C₁₋₄ alkyl-O—C(O)—O—C₁₋₄ alkyl, —C₁₋₄ alkyl-O—C(O)—C₁₋₄ alkyl-N(R^{9a})(R^{9b}), —C₁₋₄ alkyl-O—C(O)—C₁₋₄ alkyl-OP(O)(OR^{9c})₂, —C₁₋₄ alkyl-C₃₋₈ cycloalkyl, —C₁₋₄ alkyl-heterocyclyl, C₂₋₆ alkenyl, C₂₋₆ alkynyl, C₃₋₁₀ cycloalkyl, heterocyclyl, —CH₂CH(N(R^{9a})₂)C(O)OR^{9b}, C₁₋₆ alkyl-heterocyclyl, C₁₋₆ alkyl-aryl or C₁₋₆ alkyl-(5-10 membered) heteroaryl, wherein the alkyl, alkenyl, cycloalkyl, heterocyclyl, aryl, or 5-10 membered heteroaryl in each instance is optionally substituted with one to four Z^{1b};
each R^{5a} and R^{5b} is independently H, C₁₋₆ alkyl, C₁₋₆ alkyloxy, C₁₋₆ haloalkyl, C₂₋₆ alkoxyalkyl, halogen, C₃₋₁₀ cycloalkyl, heterocyclyl, —C₁₋₆ alkyl-N(R^{9a})(R^{9b}), —CN, —OR^{5a1}, or —N(R^{5a1})(R^{5a2}), or R^{5a} and R^{5b} are combined with the atoms to which they are attached to form a C₃₋₁₀ cycloalkyl or heterocyclyl, which is each optionally substituted with one to four R^{5a3};
each R^{5a1} and R^{5a2} is independently H, C₁₋₆ alkyl, C₂₋₆ alkenyl, C₂₋₆ alkynyl, C₃₋₁₀ cycloalkyl, heterocyclyl, C₆₋₁₀ aryl, or 5-10 membered heteroaryl, wherein the cycloalkyl, heterocyclyl, aryl, or 5-10 membered heteroaryl in each instance is optionally substituted with one to four halogen;
each R^{3d}, R^{5a3} is independently H, C₁₋₆ alkyl, C₁₋₆ haloalkyl, C₁₋₆ alkoxy, C₁₋₆ haloalkoxy, C₂₋₆ alkoxyalkyl, C₂₋₆ alkenyl, C₂₋₆ alkynyl, halogen, C₃₋₁₀ cycloalkyl, heterocyclyl, C₆₋₁₀ aryl, 5-10 membered heteroaryl, oxo, —OH, —CN, CO₂R^{3e}, —NO₂, or —C(O)N(R^{2a})(R^{2b}), wherein the heterocyclyl or 5-10 membered heteroaryl is optionally substituted with C₁₋₆ alkyl, C₁₋₆ haloalkyl, or C₁₋₆ haloalkoxy;
each R⁸ is independently C₁₋₉ alkyl, C₁₋₈ haloalkyl, C₂₋₆ alkenyl, C₂₋₆ alkynyl, halogen, oxo, —OH, —CN, CO₂R^{3e}, —NO₂, —NH₂, —N₃, —SH, —O(C₁₋₉ alkyl), —O(C₁₋₈ haloalkyl), —NH(C₁₋₉ alkyl), —NH(C₁₋₈ haloalkyl), —N(C₁₋₉ alkyl)₂, —N(C₁₋₈ haloalkyl)₂;
each R^{9a} and R^{9b} is independently H, C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —NO₂, —CN, —N₃, —O—R^{12a}, —C(O)R^{12a}, —C(O)O—R^{12a}, —C(O)N(R^{12a})(R^{12b}), —N(R^{12a})(R^{12b}), —N(R^{12a})₂(R^{12b})⁺, —N(R^{12a})—C(O)R^{12b}, —N(R^{12a})C(O)O(R^{12b}), —N(R^{12a})C(O)N(R^{12b})(R^{12c}), —N(R^{12a})S(O)₂(R^{12b}), —N(R^{12a})S(O)₂—N(R^{12b})(R^{12c}), —N(R^{12a})S(O)₂O(R^{12b}), —OC(O)R^{12a}, —OC(O)OR^{12a}, —OC(O)—N(R^{12a})(R^{12b}), —S—R^{12a}, —S(O)R^{12a}, —S(O)(NH)R^{12a}, —S(O)₂R^{12a}, —S(O)₂N(R^{12a})(R^{12b}), —S(O)(NR^{12a})R^{12b}, or —Si(R^{12a})₃; or R^{9a} and R^{9b} together form a 3-10 membered heterocyclyl optionally substituted with C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —NO₂, —CN, —N₃, —O—R^{12a}, —C(O)R^{12a}, —C(O)O—R^{12a}, —C(O)N(R^{12a})(R^{12b}), —N(R^{12a})(R^{12b}), —N(R^{12a})₂(R^{12b})⁺, —N(R^{12a})—C(O)R^{12b}, —N(R^{12a})C(O)O(R^{12b}), —N(R^{12a})C(O)N(R^{12b})(R^{12c}), —N(R^{12a})S(O)₂(R^{12b}), —N(R^{12a})S(O)₂—N(R^{12b})(R^{12c}), —N(R^{12a})S(O)₂O(R^{12b}), —OC(O)R^{12a}, —OC(O)OR^{12a}, —OC(O)—N(R^{12a})(R^{12b}), —S—R^{12a}, —S(O)R^{12a}, —S(O)(NH)R^{12a}, —S(O)₂R^{12a}, —S(O)₂N(R^{12a})(R^{12b}), —S(O)(NR^{12a})R^{12b}, or —Si(R^{12a})₃;
each Z¹ is independently C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —NO₂, —N₃, —CN, or —O—R^{12a}, wherein the alkyl, haloalkyl, alkenyl, alkynyl, cycloalkyl, heterocyclyl, aryl, or 5-10 membered heteroaryl in each instance is optionally substituted with one to four Z^{1a};
each Z^{1a} is independently H, C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —NO₂, —CN, —N₃, —O—R^{12a}, —C(O)R^{12a}, —C(O)O—R^{12a}, —C(O)N(R^{12a})(R^{12b}), —N(R^{12a})(R^{12b}), —N(R^{12a})₂(R^{12b})⁺, —N(R^{12a})—C(O)R^{12b}, —N(R^{12a})C(O)O(R^{12b}), —N(R^{12a})C(O)N(R^{12b})(R^{12c}), —N(R^{12a})S(O)₂(R^{12b}), —N(R^{12a})S(O)₂—N(R^{12b})(R^{12c}), —N(R^{12a})S(O)₂O(R^{12b}), —OC(O)R^{12a}, —OC(O)OR^{12a}, —OC(O)—N(R^{12a})(R^{12b}), —S—R^{12a}, —S(O)R^{12a}, —S(O)(NH)R^{12a}, —S(O)₂R^{12a}, —S(O)₂N(R^{12a})(R^{12b}), or —S(O)(NR^{12a})R^{12b}, or —Si(R^{12a})₃, wherein the alkyl, haloalkyl, alkenyl, alkynyl, cycloalkyl, heterocyclyl, aryl, or 5-10 membered heteroaryl in each instance is optionally substituted with one to four Z^{1b};
each Z^{1b} is independently H, C₁₋₉ alkyl, C₁₋₈ haloalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —OH, —CN, CO₂R^{3e}, —NO₂, —NH₂, —N₃, —SH, —O(C₁₋₉ alkyl), —O(C₁₋₈ haloalkyl), —O(C₂₋₆ alkenyl), —O(C₂₋₆ alkynyl), —O(C₃₋₁₅ cycloalkyl), —O(heterocyclyl), —O(C₆₋₁₀ aryl), —O(5-10 membered heteroaryl), —NH(C₁₋₉ alkyl), —NH(C₁₋₈ haloalkyl), —NH(C₂₋₆ alkenyl), —NH(C₂₋₆ alkynyl), —NH(C₃₋₁₅ cycloalkyl), —NH(heterocyclyl), —NH(C₆₋₁₀ aryl), —NH(5-10 membered heteroaryl), —N(cyclo-alkyl), —N(C₁₋₉ alkyl)₂, —N(C₁₋₈ haloalkyl)₂, —N(C₂₋₆ alkenyl)₂, —N(C₂₋₆ alkynyl)₂, —N(C₃₋₁₅ cycloalkyl)₂, —N(heterocyclyl)₂, —N(C₆₋₁₀ aryl)₂, —N(5-10 membered heteroaryl)₂, —N(C₁₋₉ alkyl)(C₁₋₈ haloalkyl), —N(C₁₋₉ alkyl)(C₂₋₆ alkenyl), —N(C₁₋₉ alkyl)(C₂₋₆ alkynyl), —N(C₁₋₉ alkyl)(C₃₋₁₅ cycloalkyl), —N(C₁₋₉ alkyl)(heterocyclyl), —N(C₁₋₉ alkyl)(C₆₋₁₀ aryl), —N(C₁₋₉ alkyl)(5-10 membered heteroaryl), —C(O)(C₁₋₉ alkyl), —C(O)(C₁₋₈ haloalkyl), —C(O)(C₂₋₆ alkenyl), —C(O)(C₂₋₆ alkynyl), —C(O)(C₃₋₁₅ cycloalkyl), —C(O)(heterocyclyl), —C(O)(C₆₋₁₀ aryl), —C(O)(5-10 membered heteroaryl), —C(O)O(C₁₋₉ alkyl), —C(O)O(C₁₋₈ haloalkyl), —C(O)O(C₂₋₆ alkenyl), —C(O)O(C₂₋₆ alkynyl), —C(O)O(C₃₋₁₅ cycloalkyl), —C(O)O(heterocyclyl), —C(O)O(C₆₋₁₀ aryl), —C(O)O(5-10 membered heteroaryl), —C(O)NH₂, —C(O)NH(C₁₋₉ alkyl), —C(O)NH(C₁₋₈ haloalkyl), —C(O)NH(C₂₋₆ alkenyl), —C(O)NH(C₂₋₆ alkynyl), —C(O)NH(C₃₋₁₅ cycloalkyl), —C(O)NH(heterocyclyl), —C(O)NH(C₆₋₁₀ aryl), —C(O)NH(5-10 membered heteroaryl), —C(O)N(C₁₋₉ alkyl)₂, —C(O)N(C₁₋₈ haloalkyl)₂, —C(O)N(C₂₋₆ alkenyl)₂, —C(O)N(C₂₋₆ alkynyl)₂, —C(O)N(C₃₋₁₅ cycloalkyl)₂, —C(O)N(heterocyclyl)₂, —C(O)N(C₆₋₁₀ aryl)₂, —C(O)N(5-10 membered heteroaryl)₂, —NHC(O)(C₁₋₉ alkyl), —NHC(O)(C₁₋₈ haloalkyl), —NHC(O)(C₂₋₆ alkenyl), —NHC(O)(C₂₋₆ alkynyl), —NHC(O)(C₃₋₁₅ cycloalkyl), —NHC(O)(heterocyclyl), —NHC(O)(C₆₋₁₀ aryl), —NHC(O)(5-10 membered heteroaryl), —NHC(O)O(C₁₋₉ alkyl), —NHC(O)O(C₁₋₈ haloalkyl), —NHC(O)O(C₂₋₆ alkenyl), —NHC(O)O(C₂₋₆ alkynyl), —NHC(O)O(C₃₋₁₅ cycloalkyl), —NHC(O)O(heterocyclyl), —NHC(O)O(C₆₋₁₀ aryl), —NHC(O)O(5-10 membered heteroaryl), —NHC(O)NH(C₁₋₉ alkyl), —NHC(O)NH(C₁₋₈ haloalkyl), —NHC(O)NH(C₂₋₆ alkenyl), —NHC(O)NH(C₂₋₆ alkynyl), —NHC(O)NH(C₃₋₁₅ cycloalkyl), —NHC(O)NH(heterocyclyl), —NHC(O)NH(C₆₋₁₀ aryl), or —NHC(O)NH(5-10 membered heteroaryl), wherein the alkyl, cycloalkyl, heterocyclyl, aryl, or 5-10 membered heteroaryl in each instance is optionally substituted with one to three C₁₋₉ alkyl, C₁₋₈ haloalkyl, halogen, —OH, —NH₂, CO₂H, —O(C₁₋₉ alkyl), —O(C₁₋₈ haloalkyl), —O(C₃₋₁₅ cycloalkyl), —O(heterocyclyl), —O(aryl), —O(5-10 membered heteroaryl), —NH(C₁₋₉ alkyl), —NH(C₁₋₈ haloalkyl), —NH(C₃₋₁₅ cycloalkyl), —NH(heterocyclyl), —NH(aryl), —NH(5-10 membered heteroaryl), —N(C₁₋₉ alkyl)₂, —N(C₃₋₁₅ cycloalkyl)₂, —NHC(O)(C₁₋₈ haloalkyl), —NHC(O)(C₃₋₁₅ cycloalkyl), —NHC(O)(heterocyclyl), —NHC(O)(aryl), —NHC(O)(5-10 membered heteroaryl), —NHC(O)O(C₁₋₉ alkyl), —NHC(O)O(C₁₋₈ haloalkyl), —NHC(O)O(C₂₋₆ alkynyl), —NHC(O)O(C₃₋₁₅ cycloalkyl), —NHC(O)O(heterocyclyl), —NHC(O)O(aryl), —NHC(O)O(5-10 membered heteroaryl), or —NHC(O)NH(C₁₋₉ alkyl);
each R^{1b}, R^{1c}, R^{2a}, R^{2b}, R^{4c}, R^{9c}, R^{12a}, R^{12b}, and R^{12c} is independently H, —OH, C₁₋₉ alkyl, C₂₋₆ alkenyl, C₂₋₆ alkynyl, C₃₋₁₅ cycloalkyl, heterocyclyl, C₆₋₁₀ aryl, or 5-10 membered heteroaryl wherein the alkyl, alkenyl, alkynyl, cycloalkyl, heterocyclyl, aryl, or 5-10 membered heteroaryl in each instance is optionally substituted with one to four Z^{1b}; and
each R^{3e} is independently H, C₁₋₆ alkyl, C₁₋₆ haloalkyl, —C₁₋₄ alkyl-N(R^{9a})(R^{9b}), —C₁₋₄ alkyl-C(O)N(R^{9a})(R^{9b}), —C₁₋₄ alkyl-O—C(O)—O—C₁₋₄ alkyl, —C₁₋₄ alkyl-O—C(O)—C₁₋₄ alkyl-N(R^{9a})(R^{9b}), —C₁₋₄ alkyl-C₃₋₈ cycloalkyl, —C₁₋₄ alkyl-heterocyclyl, C₃₋₁₀ cycloalkyl, heterocyclyl, C₆₋₁₀ aryl, or 5-10 membered heteroaryl, herein the alkyl, alkenyl, cycloalkyl, heterocyclyl, aryl, or 5-10 membered heteroaryl in each instance is optionally substituted with one to four Z^{1b}, wherein each 5-10 membered heteroaryl has 5 to 12 ring members and has one to four heteroatoms, each is independently N, O, or S, and wherein each heterocyclyl has 3 to 12 ring members and has one to four heteroatoms, each is independently N, O, or S.

Another embodiment concerns compounds of Formula I-A-4 or Formula I-A-5

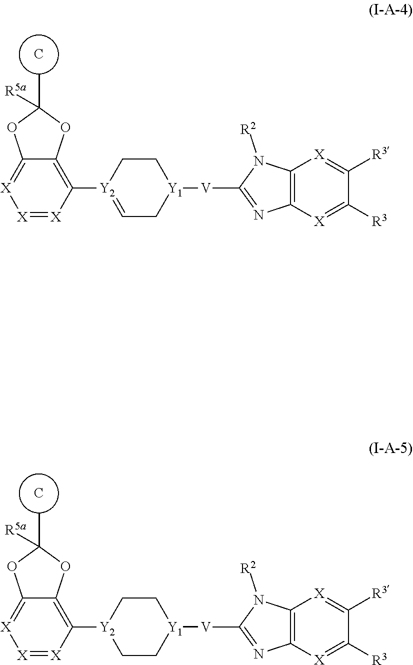

or a pharmaceutically acceptable salt thereof, wherein
Ring C is C₃₋₁₀ cycloalkyl, heterocyclyl, C₆₋₁₀ aryl or 5-10 membered heteroaryl, which is each optionally substituted with one to four Z¹;
Y₁—V is N—CH₂, N—CH(CH₃), N—CF₂, or CH—CH₂, CH—CF₂, CH—NH, N—S(O), N—S(O)₂, N—C(O), CH—C(O) or cyclopropyl;
Y₂ is C, CH or N;
R² is H, C₁₋₆ alkyl, —C₁₋₆ alkyl-alkoxy, —C₁₋₆ alkyl-cycloalkoxy, C₁₋₆ haloalkyl, C₂₋₆ alkenyl, C₂₋₆ alkynyl, C₃₋₁₀ cycloalkyl, heterocyclyl, C₆₋₁₀ aryl, 5-10 membered heteroaryl, —CN, —OR^{2a}, —S—R^{2a}, —S(O)R^{2a}, —S(O)(NH)R^{2a}, —S(O)₂R^{2a}, —S(O)₂N(R^{2a})(R^{2b}), or —S(O)(NR^{2a})R^{2b}, wherein the alkyl, alkoxy, cycloalkoxy, haloalkyl, cycloalkyl, heterocyclyl, aryl, or 5-10 membered heteroaryl in each instance is optionally substituted with one to four Z¹, any two Z₁ can be combined with the atoms to which they are attached to form a C₃₋₁₀ cycloalkyl or heterocyclyl or C₆₋₁₀ aryl or 5-10 membered heteroaryl;
X is each independently —N═, —C(H)═, or —C(R⁸)═;
each R³ and R^{3′} is independently H, C₁₋₆ alkyl, C₁₋₆ haloalkyl, C₂₋₆ alkenyl, C₂₋₆ alkynyl, halogen, C₃₋₁₀ cycloalkyl, heterocyclyl, C₆₋₁₀ aryl, 5-10 membered heteroaryl, —CN, —NO₂, —OR^{3a}, —C(O)R^{3a}, —CH₂C(O)OR^{3a}, —C(O)OR^{3a}, —C(O)N(R^{3a})(R^{3b}), —N(R^{3a})C(O)R^{3b}, —N(R^{3a})C(O)OR^{3b}, —N(R^{3a})C(O)N(R^{3b})₂, —C(O)NHS(O)₂R^{3a}, —C(O)NR^{3a}S(O)₂R^{3b}, —C(O)NR^{3a}S(O)₂NR^{3b}R^{3c}, —C(O)NR^{3a}—S(O)(═NR^{3b})R^{3c}, —S(O)₂R^{3a}, —S(O)₂OR^{3a}, —S(O)₂N(R^{3a})(R^{3b}), —N(R^{3a})S(O)₂R^{3b}, —S(O)₂NHC(O)R^{3a}, —S(O)(═NR^{3a})R^{3b}, —S(O)(═NR^{3a})NR^{3b}, —S(═NR^{3a})(═NR^{3b})R^{3c}, —P(O)(OR^{3a})(R^{3b}), —P(O)(OR^{3a})(OR^{3b}), —B(OR^{3a})(OR^{3b}), or —O—C₁₋₆alkyl-C(O)OR^{3a}, wherein the alkyl, haloalkyl, alkenyl, alkynyl, cycloalkyl, heterocyclyl, aryl, or 5-10 membered heteroaryl in each instance is optionally substituted with one to four R^{3d};
each R^{3a}, R^{3b}, and R^{3c} is independently H, C₁₋₆ alkyl, C₆₋₁₀ aryl, 5-10 membered heteroaryl, C₁₋₆ haloalkyl, C₂₋₈ alkoxyalkyl, —C₁₋₄ alkyl-N(R^{9a})(R^{9b}), —C₁₋₄ alkyl-N(R^{9a})C(O)—O—C₁₋₄ alkyl-OP(O)(OR^{9c})₂, —C₁₋₄ alkyl-C(O)N(R^{9a})(R^{9b}), —C₁₋₄ alkyl-O—C(O)—O—C₁₋₄ alkyl, —C₁₋₄ alkyl-O—C(O)—C₁₋₄ alkyl-N(R^{9a})(R^{9b}), —C₁₋₄ alkyl-O—C(O)—C₁₋₄ alkyl-OP(O)(OR^{9c})₂, —C₁₋₄ alkyl-C₃₋₈ cycloalkyl, —C₁₋₄ alkyl-heterocyclyl, C₂₋₆ alkenyl, C₂₋₆ alkynyl, C₃₋₁₀ cycloalkyl, heterocyclyl, —CH₂CH(N(R^{9a})₂)C(O)OR^{9b}, C₁₋₆ alkyl-heterocyclyl, C₁₋₆ alkyl-aryl or C₁₋₆ alkyl-(5-10 membered) heteroaryl, wherein the alkyl, alkenyl, cycloalkyl, heterocyclyl, aryl, or 5-10 membered heteroaryl in each instance is optionally substituted with one to four Z^{1b};
R^{5a} is each independently H, C₁₋₆ alkyl, C₁₋₆ alkyloxy, C₁₋₆ haloalkyl, C₂₋₆ alkoxyalkyl, halogen, C₃₋₁₀ cycloalkyl, heterocyclyl, —C₁₋₆ alkyl-N(R^{9a})(R^{9b}), —CN, —OR^{5a1}, or —N(R^{5a1})(R^{5a2});
each R^{5a1} and R^{5a2} is independently H, C₁₋₆ alkyl, C₂₋₆ alkenyl, C₂₋₆ alkynyl, C₃₋₁₀ cycloalkyl, heterocyclyl, C₆₋₁₀ aryl, or 5-10 membered heteroaryl, wherein the cycloalkyl, heterocyclyl, aryl, or 5-10 membered heteroaryl in each instance is optionally substituted with one to four halogen;
R^{3d} is independently H, C₁₋₆ alkyl, C₁₋₆ haloalkyl, C₁₋₆ alkoxy, C₁₋₆ haloalkoxy, C₂₋₆ alkoxyalkyl, C₂₋₆ alkenyl, C₂₋₆ alkynyl, halogen, C₃₋₁₀ cycloalkyl, heterocyclyl, C₆₋₁₀ aryl, 5-10 membered heteroaryl, oxo, —OH, —CN, CO₂R^{3e}, —NO₂, or —C(O)N(R^{2a})(R^{2b}), wherein the heterocyclyl or 5-10 membered heteroaryl is optionally substituted with C₁₋₆ alkyl, C₁₋₆ haloalkyl, or C₁₋₆ haloalkoxy;
each R⁸ is independently C₁₋₉ alkyl, C₁₋₈ haloalkyl, C₂₋₆ alkenyl, C₂₋₆ alkynyl, halogen, oxo, —OH, —CN, CO₂R^{3e}, —NO₂, —NH₂, —N₃, —SH, —O(C₁₋₉ alkyl), —O(C₁₋₈ haloalkyl), —NH(C₁₋₉ alkyl), —NH(C₁₋₈ haloalkyl), —N(C₁₋₉ alkyl)₂, —N(C₁₋₈ haloalkyl)₂;
each R^{9a} and R^{9b} is independently H, C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —NO₂, —CN, —N₃, —O—R^{12a}, —C(O)R^{12a}, —C(O)O—R^{12a}, —C(O)N(R^{12a})(R^{12b}), —N(R^{12a})(R^{12b}), —N(R^{12a})₂(R^{12b})⁺, —N(R^{12a})—C(O)R^{12b}, —N(R^{12a})C(O)O(R^{12b}), —N(R^{12a})C(O)N(R^{12b})(R^{12c}), —N(R^{12a})S(O)₂(R^{12b}), —N(R^{12a})S(O)₂—N(R^{12b})(R^{12c}), —N(R^{12a})S(O)₂O(R^{12b}), —OC(O)R^{12a}, —OC(O)OR^{12a}, —OC(O)—N(R^{12a})(R^{12b}), —S—R^{12a}, —S(O)R^{12a}, —S(O)(NH)R^{12a}, —S(O)₂R^{12a}, —S(O)₂N(R^{12a})(R^{12b}), —S(O)(NR^{12a})R^{12b}, or —Si(R^{12a})₃; or R^{9a} and R^{9b} together form a 3-10 membered heterocyclyl optionally substituted with C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —NO₂, —CN, —N₃, —O—R^{12a}, —C(O)R^{12a}, —C(O)O—R^{12a}, —C(O)N(R^{12a})(R^{12b}), —N(R^{12a})(R^{12b}), —N(R^{12a})₂(R^{12b})⁺, —N(R^{12a})—C(O)R^{12b}, —N(R^{12a})C(O)O(R^{12b}), —N(R^{12a})C(O)N(R^{12b})(R^{12c}), —N(R^{12a})S(O)₂(R^{12b}), —N(R^{12a})S(O)₂—N(R^{12b})(R^{12c}), —N(R^{12a})S(O)₂O(R^{12b}), —OC(O)R^{12a}, —OC(O)OR^{12a}, —OC(O)—N(R^{12a})(R^{12b}), —S—R^{12a}, —S(O)R^{12a}, —S(O)(NH)R^{12a}, —S(O)₂R^{12a}, —S(O)₂N(R^{12a})(R^{12b}), —S(O)(NR^{12a})R^{12b}, or —Si(R^{12a})₃;
each Z¹ is independently C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —NO₂, —N₃, —CN, or —O—R^{12a}, wherein the alkyl, haloalkyl, alkenyl, alkynyl, cycloalkyl, heterocyclyl, aryl, or 5-10 membered heteroaryl in each instance is optionally substituted with one to four Z^{1a};
each Z^{1a} is independently H, C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —NO₂, —CN, —N₃, —O—R^{12a}, —C(O)R^{12a}, —C(O)O—R^{12a}, —C(O)N(R^{12a})(R^{12b}), —N(R^{12a})(R^{12b}), —N(R^{12a})₂(R^{12b})⁺, —N(R^{12a})—C(O)R^{12b}, —N(R^{12a})C(O)O(R^{12b}), —N(R^{12a})C(O)N(R^{12b})(R^{12c}), —N(R^{12a})S(O)₂(R^{12b}), —N(R^{12a})S(O)₂—N(R^{12b})(R^{12c}), —N(R^{12a})S(O)₂O(R^{12b}), —OC(O)R^{12a}, —OC(O)OR^{12a}, —OC(O)—N(R^{12a})(R^{12b}), —S—R^{12a}, —S(O)R^{12a}, —S(O)(NH)R^{12a}, —S(O)₂R^{12a}, —S(O)₂N(R^{12a})(R^{12b}), —S(O)(NR^{12a})R^{12b}, or —Si(R^{12a})₃, wherein the alkyl, haloalkyl, alkenyl, alkynyl, cycloalkyl, heterocyclyl, aryl, or 5-10 membered heteroaryl in each instance is optionally substituted with one to four Z^{1b};
each Z^{1b} is independently H, C₁₋₉ alkyl, C₁₋₈ haloalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —OH, —CN, CO₂R^{3e}, —NO₂, —NH₂, —N₃, —SH, —O(C₁₋₉ alkyl), —O(C₁₋₈ haloalkyl), —O(C₂₋₆ alkenyl), —O(C₂₋₆ alkynyl), —O(C₃₋₁₅ cycloalkyl), —O(heterocyclyl), —O(C₆₋₁₀ aryl), —O(5-10 membered heteroaryl), —NH(C₁₋₉ alkyl), —NH(C₁₋₈ haloalkyl), —NH(C₂₋₆ alkenyl), —NH(C₂₋₆ alkynyl), —NH(C₃₋₁₅ cycloalkyl), —NH(heterocyclyl), —NH(C₆₋₁₀ aryl), —NH(5-10 membered heteroaryl), —N(cyclo-alkyl), —N(C₁₋₉ alkyl)₂, —N(C₁₋₈ haloalkyl)₂, —N(C₂₋₆ alkenyl)₂, —N(C₂₋₆ alkynyl)₂, —N(C₃₋₁₅ cycloalkyl)₂, —N(heterocyclyl)₂, —N(C₆₋₁₀ aryl)₂, —N(5-10 membered heteroaryl)₂, —N(C₁₋₉ alkyl)(C₁₋₈ haloalkyl), —N(C₁₋₉ alkyl)(C₂₋₆ alkenyl), —N(C₁₋₉ alkyl)(C₂₋₆ alkynyl), —N(C₁₋₉ alkyl)(C₃₋₁₅ cycloalkyl), —N(C₁₋₉ alkyl)(heterocyclyl), —N(C₁₋₉ alkyl)(C₆₋₁₀ aryl), —N(C₁₋₉ alkyl)(5-10 membered heteroaryl), —C(O)(C₁₋₉ alkyl), —C(O)(C₁₋₈ haloalkyl), —C(O)(C₂₋₆ alkenyl), —C(O)(C₂₋₆ alkynyl), —C(O)(C₃₋₁₅ cycloalkyl), —C(O)(heterocyclyl), —C(O)(C₆₋₁₀ aryl), —C(O)(5-10 membered heteroaryl), —C(O)O(C₁₋₉ alkyl), —C(O)O(C₁₋₈ haloalkyl), —C(O)O(C₂₋₆ alkenyl), —C(O)O(C₂₋₆ alkynyl), —C(O)O(C₃₋₁₅ cycloalkyl), —C(O)O(heterocyclyl), —C(O)O(C₆₋₁₀ aryl), —C(O)O(5-10 membered heteroaryl), —C(O)NH₂, —C(O)NH(C₁₋₉ alkyl), —C(O)NH(C₁₋₈ haloalkyl), —C(O)NH(C₂₋₆ alkenyl), —C(O)NH(C₂₋₆ alkynyl), —C(O)NH(C₃₋₁₅ cycloalkyl), —C(O)NH(heterocyclyl), —C(O)NH(C₆₋₁₀ aryl), —C(O)NH(5-10 membered heteroaryl), —C(O)N(C₁₋₉ alkyl)₂, —C(O)N(C₁₋₈ haloalkyl)₂, —C(O)N(C₂₋₆ alkenyl)₂, —C(O)N(C₂₋₆ alkynyl)₂, —C(O)N(C₃₋₁₅ cycloalkyl)₂, —C(O)N(heterocyclyl)₂, —C(O)N(C₆₋₁₀ aryl)₂, —C(O)N(5-10 membered heteroaryl)₂, —NHC(O)(C₁₋₉ alkyl), —NHC(O)(C₁₋₈ haloalkyl), —NHC(O)(C₂₋₆ alkenyl), —NHC(O)(C₂₋₆ alkynyl), —NHC(O)(C₃₋₁₅ cycloalkyl), —NHC(O)(heterocyclyl), —NHC(O)(C₆₋₁₀ aryl), —NHC(O)(5-10 membered heteroaryl), —NHC(O)O(C₁₋₉ alkyl), —NHC(O)O(C₁₋₈ haloalkyl), —NHC(O)O(C₂₋₆ alkenyl), —NHC(O)O(C₂₋₆ alkynyl), —NHC(O)O(C₃₋₁₅ cycloalkyl), —NHC(O)O(heterocyclyl), —NHC(O)O(C₆₋₁₀ aryl), —NHC(O)O(5-10 membered heteroaryl), —NHC(O)NH(C₁₋₉ alkyl), —NHC(O)NH(C₁₋₈ haloalkyl), —NHC(O)NH(C₂₋₆ alkenyl), —NHC(O)NH(C₂₋₆ alkynyl), —NHC(O)NH(C₃₋₁₅ cycloalkyl), —NHC(O)NH(heterocyclyl), —NHC(O)NH(C₆₋₁₀ aryl), or —NHC(O)NH(5-10 membered heteroaryl), wherein the alkyl, cycloalkyl, heterocyclyl, aryl, or 5-10 membered heteroaryl in each instance is optionally substituted with one to three C₁₋₉ alkyl, C₁₋₈ haloalkyl, halogen, —OH, —NH₂, CO₂H, —O(C₁₋₉ alkyl), —O(C₁₋₈ haloalkyl), —O(C₃₋₁₅ cycloalkyl), —O(heterocyclyl), —O(aryl), —O(5-10 membered heteroaryl), —NH(C₁₋₉ alkyl), —NH(C₁₋₈ haloalkyl), —NH(C₃₋₁₅ cycloalkyl), —NH(heterocyclyl), —NH(aryl), —NH(5-10 membered heteroaryl), —N(C₁₋₉ alkyl)₂, —N(C₃₋₁₅ cycloalkyl)₂, —NHC(O)(C₁₋₈ haloalkyl), —NHC(O)(C₃₋₁₅ cycloalkyl), —NHC(O)(heterocyclyl), —NHC(O)(aryl), —NHC(O)(5-10 membered heteroaryl), —NHC(O)O(C₁₋₉ alkyl), —NHC(O)O(C₁₋₈ haloalkyl), —NHC(O)O(C₂₋₆ alkynyl), —NHC(O)O(C₃₋₁₅ cycloalkyl), —NHC(O)O(heterocyclyl), —NHC(O)O(aryl), —NHC(O)O(5-10 membered heteroaryl), or —NHC(O)NH(C₁₋₉ alkyl);
each R^{1b}, R^{1c}, R^{2a}, R^{2b}, R^{4c}, R^{9c}, R^{12a}, R^{12b}, and R^{12c} is independently H, —OH, C₁₋₉ alkyl, C₂₋₆ alkenyl, C₂₋₆ alkynyl, C₃₋₁₅ cycloalkyl, heterocyclyl, C₆₋₁₀ aryl, or 5-10 membered heteroaryl, wherein the alkyl, alkenyl, alkynyl, cycloalkyl, heterocyclyl, aryl, or 5-10 membered heteroaryl in each instance is optionally substituted with one to four Z^{1b}; and
each R^{3e} is independently H, C₁₋₆ alkyl, C₁₋₆ haloalkyl, —C₁₋₄ alkyl-N(R^{9a})(R^{9b}), —C₁₋₄ alkyl-C(O)N(R^{9a})(R^{9b}), —C₁₋₄ alkyl-O—C(O)—O—C₁₋₄ alkyl, —C₁₋₄ alkyl-O—C(O)—C₁₋₄ alkyl-N(R^{9a})(R^{9b}), —C₁₋₄ alkyl-C₃₋₈ cycloalkyl, —C₁₋₄ alkyl-heterocyclyl, C₃₋₁₀ cycloalkyl, heterocyclyl, C₆₋₁₀ aryl, or 5-10 membered heteroaryl, wherein the alkyl, alkenyl, cycloalkyl, heterocyclyl, aryl, or 5-10 membered heteroaryl in each instance is optionally substituted with one to four Z^{1b}, wherein each 5-10 membered heteroaryl has 5 to 12 ring members and has one to four heteroatoms, each is independently N, O, or S; and wherein each heterocyclyl has 3 to 12 ring members and has one to four heteroatoms, each is independently N, O, or S.

Another embodiment concerns compounds of Formula I-A-6 or Formula I-A-7

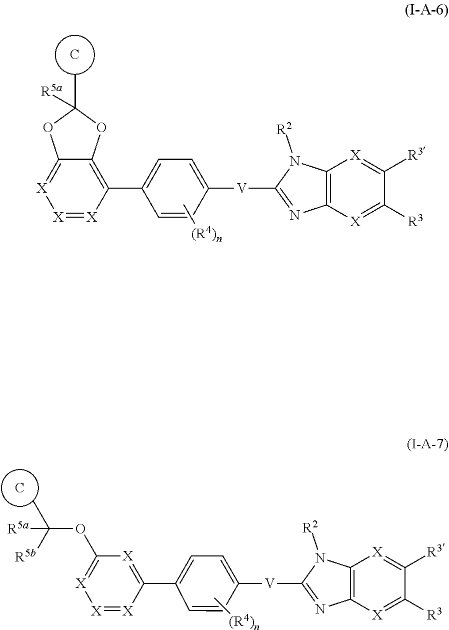

or a pharmaceutically acceptable salt thereof, wherein
Ring C is C₃₋₁₀ cycloalkyl, heterocyclyl, C₆₋₁₀ aryl or 5-10 membered heteroaryl, which is each optionally substituted with one to four Z¹;
V is CH₂, CH(CH₃), NH, C(O), S(O), S(O)₂;
n=0, 1, 2, 3, or 4;
R² is H, C₁₋₆ alkyl, —C₁₋₆ alkyl-alkoxy, —C₁₋₆ alkyl-cycloalkoxy, C₁₋₆ haloalkyl, C₂₋₆ alkenyl, C₂₋₆ alkynyl, C₃₋₁₀ cycloalkyl, heterocyclyl, C₆₋₁₀ aryl, 5-10 membered heteroaryl, —CN, —OR^{2a}, —S—R^{2a}, —S(O)R^{2a}, —S(O)(NH)R^{2a}, —S(O)₂R^{2a}, —S(O)₂N(R^{2a})(R^{2b}), or —S(O)(NR^{2a})R^{2b}, wherein the alkyl, alkoxy, cycloalkoxy, haloalkyl, cycloalkyl, heterocyclyl, aryl, or 5-10 membered heteroaryl in each instance is optionally substituted with one to four Z¹, and wherein any two Z¹ can be combined with the atoms to which they are attached to form a C₃₋₁₀ cycloalkyl or heterocyclyl or C₆₋₁₀ aryl or 5-10 membered heteroaryl;
X is each independently —N═, —C(H)═, or —C(R⁸)═;
each R³ and R^{3′} is independently H, C₁₋₆ alkyl, C₁₋₆ haloalkyl, C₂₋₆ alkenyl, C₂₋₆ alkynyl, halogen, C₃₋₁₀ cycloalkyl, heterocyclyl, C₆₋₁₀ aryl, 5-10 membered heteroaryl, —CN, —NO₂, —OR^{3a}, —C(O)R^{3a}, —CH₂C(O)OR^{3a}, —C(O)OR^{3a}, —C(O)N(R^{3a})(R^{3b}), —N(R^{3a})C(O)R^{3b}, —N(R^{3a})C(O)OR^{3b}, —N(R^{3a})C(O)N(R^{3b})₂, —C(O)NHS(O)₂R^{3a}, —C(O)NR^{3a}S(O)₂R^{3b}, —C(O)NR^{3a}S(O)₂NR^{3b}R^{3c}, —C(O)NR^{3a}—S(O)(═NR^{3b})R^{3c}, —S(O)₂R^{3a}, —S(O)₂OR^{3a}, —S(O)₂N(R^{3a})(R^{3b}), —N(R^{3a})S(O)₂R^{3b}, —S(O)₂NHC(O)R^{3a}, —S(O)(═NR^{3a})R^{3b}, —S(O)(═NR^{3a})NR^{3b}, —S(═NR^{3a})(═NR^{3b})R^{3c}, —P(O)(OR^{3a})(R^{3b}), —P(O)(OR^{3a})(OR^{3b}), —B(OR^{3a})(OR^{3b}), or —O—C₁₋₆alkyl-C(O)OR^{3a}, wherein the alkyl, haloalkyl, alkenyl, alkynyl, cycloalkyl, heterocyclyl, aryl, or 5-10 membered heteroaryl in each instance is optionally substituted with one to four R^{3d};
each R^{3a}, R^{3b}, and R^{3c} is independently H, C₁₋₆ alkyl, C₆₋₁₀ aryl, 5-10 membered heteroaryl, C₁₋₆ haloalkyl, C₂₋₈ alkoxyalkyl, —C₁₋₄ alkyl-N(R^{9a})(R^{9b}), —C₁₋₄ alkyl-N(R^{9a})C(O)—O—C₁₋₄ alkyl-OP(O)(OR^{9c})₂, —C₁₋₄ alkyl-C(O)N(R^{9a})(R^{9b}), —C₁₋₄ alkyl-O—C(O)—O—C₁₋₄ alkyl, —C₁₋₄ alkyl-O—C(O)—C₁₋₄ alkyl-N(R^{9a})(R^{9b}), —C₁₋₄ alkyl-O—C(O)—C₁₋₄ alkyl-OP(O)(OR^{9c})₂, —C₁₋₄ alkyl-C₃₋₈ cycloalkyl, —C₁₋₄ alkyl-heterocyclyl, C₂₋₆ alkenyl, C₂₋₆ alkynyl, C₃₋₁₀ cycloalkyl, heterocyclyl, C₆₋₁₀ aryl, 5-10 membered heteroaryl, —CH₂CH(N(R^{9a})₂)C(O)OR^{9b}, C₁₋₆ alkyl-heterocyclyl, C₁₋₆ alkyl-aryl or C₁₋₆ alkyl-(5-10 membered) heteroaryl, wherein the alkyl, alkenyl, cycloalkyl, heterocyclyl, aryl, or 5-10 membered heteroaryl in each instance is optionally substituted with one to four Z^{1b};
each R⁴ is independently H, —OH, CH₂OH, C₁₋₉ alkyl, C₁₋₈ haloalkyl, C₁₋₆ haloalkoxy, C₂₋₆ alkoxyalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —NO₂, —CN, —N₃, —O—R^{4a}, —C(O)R^{4a}, —C(O)O—R^{4a}, —C(O)N(R^{4a})(R^{4b}), —N(R^{4a})(R^{4b}), —N(R^{4a})₂(R^{4b})⁺, —N(R^{4a})—C(O)R^{4b}, —N(R^{4a})C(O)O(R^{4b}), —N(R^{4a})C(O)N(R^{4b})(R^{4c}), —N(R^{4a})S(O)₂(R^{4b}), —N(R^{4a})S(O)₂—N(R^{4b})(R^{4c}), —N(R^{4a})S(O)₂O(R^{4b}), —OC(O)R^{4a}, —OC(O)OR^{4a}, —OC(O)—N(R^{4a})(R^{4b}), —S—R^{4a}, —S(O)R^{4a}, —S(O)(NH)R^{4a}, —S(O)₂R^{4a}, —S(O)₂N(R^{4a})(R^{4b}), —S(O)(NR^{4a})R^{4b}, or —Si(R^{4a})₃, wherein the alkyl, haloalkyl, alkenyl, alkynyl, cycloalkyl, heterocyclyl, aryl, or 5-10 membered heteroaryl in each instance is optionally substituted with one to four Z^{1b}; or two R⁴ groups attached to adjacent ring atoms are combined with the atoms to which they are attached to form a C₅₋₁₀ cycloalkyl or heterocyclyl, which is each optionally substituted with one to four Z^{1b};
each R^{5a} and R^{5b} is independently H, C₁₋₆ alkyl, C₁₋₆ alkyloxy, C₁₋₆ haloalkyl, C₂₋₆ alkoxyalkyl, halogen, C₃₋₁₀ cycloalkyl, heterocyclyl, —C₁₋₆ alkyl-N(R^{9a})(R^{9b}), —CN, —OR^{5a1}, or —N(R^{5a1})(R^{5a2}), or R^{5a} and R^{5b} are combined with the atoms to which they are attached to form a C₃₋₁₀ cycloalkyl or heterocyclyl, which is each optionally substituted with one to four R^{5a3};
each R^{5a1} and R^{5a2} is independently H, C₁₋₆ alkyl, C₂₋₆ alkenyl, C₂₋₆ alkynyl, C₃₋₁₀ cycloalkyl, heterocyclyl, C₆₋₁₀ aryl, or 5-10 membered heteroaryl, wherein the cycloalkyl, heterocyclyl, aryl, or 5-10 membered heteroaryl in each instance is optionally substituted with one to four halogen;
each R^{3d} and R^{5a3} is independently H, C₁₋₆ alkyl, C₁₋₆ haloalkyl, C₁₋₆ alkoxy, C₁₋₆ haloalkoxy, C₂₋₆ alkoxyalkyl, C₂₋₆ alkenyl, C₂₋₆ alkynyl, halogen, C₃₋₁₀ cycloalkyl, heterocyclyl, C₆₋₁₀ aryl, 5-10 membered heteroaryl, oxo, —OH, —CN, CO₂R^{3e}, —NO₂, or —C(O)N(R^{2a})(R^{2b}), wherein the heterocyclyl or 5-10 membered heteroaryl in each instance is optionally substituted with C₁₋₆ alkyl, C₁₋₆ haloalkyl, or C₁₋₆ haloalkoxy;
each R⁸ is independently C₁₋₉ alkyl, C₁₋₈ haloalkyl, C₂₋₆ alkenyl, C₂₋₆ alkynyl, halogen, oxo, —OH, —CN, CO₂R^{3e}, —NO₂, —NH₂, —N₃, —SH, —O(C₁₋₉ alkyl), —O(C₁₋₈ haloalkyl), —NH(C₁₋₉ alkyl), —NH(C₁₋₈ haloalkyl), —N(C₁₋₉ alkyl)₂, —N(C₁₋₈ haloalkyl)₂;
each R^{9a} and R^{9b} is independently H, C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —NO₂, —CN, —N₃, —O—R^{12a}, —C(O)R^{12a}, —C(O)O—R^{12a}, —C(O)N(R^{12a})(R^{12b}), —N(R^{12a})(R^{12b}), —N(R^{12a})₂(R^{12b})⁺, —N(R^{12a})—C(O)R^{12b}, —N(R^{12a})C(O)O(R^{12b}), —N(R^{12a})C(O)N(R^{12b})(R^{12c}), —N(R^{12a})S(O)₂(R^{12b}), —N(R^{12a})S(O)₂—N(R^{12b})(R^{12c}), —N(R^{12a})S(O)₂O(R^{12b}), —OC(O)R^{12a}, —OC(O)OR^{12a}, —OC(O)—N(R^{12a})(R^{12b}), —S—R^{12a}, —S(O)R^{12a}, —S(O)(NH)R^{12a}, —S(O)₂R^{12a}, —S(O)₂N(R^{12a})(R^{12b}), —S(O)(NR^{12a})R^{12b}, or —Si(R^{12a})₃; or R^{9a} and R^{9b} together form a 3-10 membered heterocyclyl optionally substituted with C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —NO₂, —CN, —N₃, —O—R^{12a}, —C(O)R^{12a}, —C(O)O—R^{12a}, —C(O)N(R^{12a})(R^{12b}), —N(R^{12a})(R^{12b}), —N(R^{12a})₂(R^{12b})⁺, —N(R^{12a})—C(O)R^{12b}, —N(R^{12a})C(O)O(R^{12b}), —N(R^{12a})C(O)N(R^{12b})(R^{12c}), —N(R^{12a})S(O)₂(R^{12b}), —N(R^{12a})S(O)₂—N(R^{12b})(R^{12c}), —N(R^{12a})S(O)₂O(R^{12b}), —OC(O)R^{12a}, —OC(O)OR^{12a}, —OC(O)—N(R^{12a})(R^{12b}), —S—R^{12a}, —S(O)R^{12a}, —S(O)(NH)R^{12a}, —S(O)₂R^{12a}, —S(O)₂N(R^{12a})(R^{12b}), —S(O)(NR^{12a})R^{12b}, or —Si(R^{12a})₃;
each Z¹ is independently C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —NO₂, —N₃, —CN, —O—R^{12a}, wherein the alkyl, haloalkyl, alkenyl, alkynyl, cycloalkyl, heterocyclyl, aryl, or 5-10 membered heteroaryl in each instance is optionally substituted with one to four Z^{1a};
each Z^{1a} is independently H, C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —NO₂, —CN, —N₃, —O—R^{12a}, —C(O)R^{12a}, —C(O)O—R^{12a}, —C(O)N(R^{12a})(R^{12b}), —N(R^{12a})(R^{12b}), —N(R^{12a})₂(R^{12b})⁺, —N(R^{12a})—C(O)R^{12b}, —N(R^{12a})C(O)O(R^{12b}), —N(R^{12a})C(O)N(R^{12b})(R^{12c}), —N(R^{12a})S(O)₂(R^{12b}), —N(R^{12a})S(O)₂—N(R^{12b})(R^{12c}), —N(R^{12a})S(O)₂O(R^{12b}), —OC(O)R^{12a}, —OC(O)OR^{12a}, —OC(O)—N(R^{12a})(R^{12b}), —S—R^{12a}, —S(O)R^{12a}, —S(O)(NH)R^{12a}, —S(O)₂R^{12a}, —S(O)₂N(R^{12a})(R^{12b}), —S(O)(NR^{12a})R^{12b}, or —Si(R^{12a})₃, wherein the alkyl, haloalkyl, alkenyl, alkynyl, cycloalkyl, heterocyclyl, aryl, or 5-10 membered heteroaryl in each instance is optionally substituted with one to four Z^{1b};
each Z^{1b} is independently H, C₁₋₉ alkyl, C₁₋₈ haloalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —OH, —CN, CO₂R^{3e}, —NO₂, —NH₂, —N₃, —SH, —O(C₁₋₉ alkyl), —O(C₁₋₈ haloalkyl), —O(C₂₋₆ alkenyl), —O(C₂₋₆ alkynyl), —O(C₃₋₁₅ cycloalkyl), —O(heterocyclyl), —O(C₆₋₁₀ aryl), —O(5-10 membered heteroaryl), —NH(C₁₋₉ alkyl), —NH(C₁₋₈ haloalkyl), —NH(C₂₋₆ alkenyl), —NH(C₂₋₆ alkynyl), —NH(C₃₋₁₅ cycloalkyl), —NH(heterocyclyl), —NH(C₆₋₁₀ aryl), —NH(5-10 membered heteroaryl), —N(cyclo-alkyl), —N(C₁₋₉ alkyl)₂, —N(C₁₋₈ haloalkyl)₂, —N(C₂₋₆ alkenyl)₂, —N(C₂₋₆ alkynyl)₂, —N(C₃₋₁₅ cycloalkyl)₂, —N(heterocyclyl)₂, —N(C₆₋₁₀ aryl)₂, —N(5-10 membered heteroaryl)₂, —N(C₁₋₉ alkyl)(C₁₋₈ haloalkyl), —N(C₁₋₉ alkyl)(C₂₋₆ alkenyl), —N(C₁₋₉ alkyl)(C₂₋₆ alkynyl), —N(C₁₋₉ alkyl)(C₃₋₁₅ cycloalkyl), —N(C₁₋₉ alkyl)(heterocyclyl), —N(C₁₋₉ alkyl)(C₆₋₁₀ aryl), —N(C₁₋₉ alkyl)(5-10 membered heteroaryl), —C(O)(C₁₋₉ alkyl), —C(O)(C₁₋₈ haloalkyl), —C(O)(C₂₋₆ alkenyl), —C(O)(C₂₋₆ alkynyl), —C(O)(C₃₋₁₅ cycloalkyl), —C(O)(heterocyclyl), —C(O)(C₆₋₁₀ aryl), —C(O)(5-10 membered heteroaryl), —C(O)O(C₁₋₉ alkyl), —C(O)O(C₁₋₈ haloalkyl), —C(O)O(C₂₋₆ alkenyl), —C(O)O(C₂₋₆ alkynyl), —C(O)O(C₃₋₁₅ cycloalkyl), —C(O)O(heterocyclyl), —C(O)O(C₆₋₁₀ aryl), —C(O)O(5-10 membered heteroaryl), —C(O)NH₂, —C(O)NH(C₁₋₉ alkyl), —C(O)NH(C₁₋₈ haloalkyl), —C(O)NH(C₂₋₆ alkenyl), —C(O)NH(C₂₋₆ alkynyl), —C(O)NH(C₃₋₁₅ cycloalkyl), —C(O)NH(heterocyclyl), —C(O)NH(C₆₋₁₀ aryl), —C(O)NH(5-10 membered heteroaryl), —C(O)N(C₁₋₉ alkyl)₂, —C(O)N(C₁₋₈ haloalkyl)₂, —C(O)N(C₂₋₆ alkenyl)₂, —C(O)N(C₂₋₆ alkynyl)₂, —C(O)N(C₃₋₁₅ cycloalkyl)₂, —C(O)N(heterocyclyl)₂, —C(O)N(C₆₋₁₀ aryl)₂, —C(O)N(5-10 membered heteroaryl)₂, —NHC(O)(C₁₋₉ alkyl), —NHC(O)(C₁₋₈ haloalkyl), —NHC(O)(C₂₋₆ alkenyl), —NHC(O)(C₂₋₆ alkynyl), —NHC(O)(C₃₋₁₅ cycloalkyl), —NHC(O)(heterocyclyl), —NHC(O)(C₆₋₁₀ aryl), —NHC(O)(5-10 membered heteroaryl), —NHC(O)O(C₁₋₉ alkyl), —NHC(O)O(C₁₋₈ haloalkyl), —NHC(O)O(C₂₋₆ alkenyl), —NHC(O)O(C₂₋₆ alkynyl), —NHC(O)O(C₃₋₁₅ cycloalkyl), —NHC(O)O(heterocyclyl), —NHC(O)O(C₆₋₁₀ aryl), —NHC(O)O(5-10 membered heteroaryl), —NHC(O)NH(C₁₋₉ alkyl), —NHC(O)NH(C₁₋₈ haloalkyl), —NHC(O)NH(C₂₋₆ alkenyl), —NHC(O)NH(C₂₋₆ alkynyl), —NHC(O)NH(C₃₋₁₅ cycloalkyl), —NHC(O)NH(heterocyclyl), —NHC(O)NH(C₆₋₁₀ aryl), or —NHC(O)NH(5-10 membered heteroaryl), wherein the alkyl, cycloalkyl, heterocyclyl, aryl, or 5-10 membered heteroaryl in each instance is optionally substituted with one to three C₁₋₉ alkyl, C₁₋₈ haloalkyl, halogen, —OH, —NH₂, CO₂H, —O(C₁₋₉ alkyl), —O(C₁₋₈ haloalkyl), —O(C₃₋₁₅ cycloalkyl), —O(heterocyclyl), —O(aryl), —O(5-10 membered heteroaryl), —NH(C₁₋₉ alkyl), —NH(C₁₋₈ haloalkyl), —NH(C₃₋₁₅ cycloalkyl), —NH(heterocyclyl), —NH(aryl), —NH(5-10 membered heteroaryl), —N(C₁₋₉ alkyl)₂, —N(C₃₋₁₅ cycloalkyl)₂, —NHC(O)(C₁₋₈ haloalkyl), —NHC(O)(C₃₋₁₅ cycloalkyl), —NHC(O)(heterocyclyl), —NHC(O)(aryl), —NHC(O)(5-10 membered heteroaryl), —NHC(O)O(C₁₋₉ alkyl), —NHC(O)O(C₁₋₈ haloalkyl), —NHC(O)O(C₂₋₆ alkynyl), —NHC(O)O(C₃₋₁₅ cycloalkyl), —NHC(O)O(heterocyclyl), —NHC(O)O(aryl), —NHC(O)O(5-10 membered heteroaryl), or —NHC(O)NH(C₁₋₉ alkyl;
each R^{1b}, R^{1c}, R^{2a}, R^{2b}, R^{4a}, R^{4b}, R^{4c}, R^{9c}, R^{12a}, R^{12b}, and R^{12c} is independently H, —OH, C₁₋₉ alkyl, C₂₋₆ alkenyl, C₂₋₆ alkynyl, C₃₋₁₅ cycloalkyl, heterocyclyl, C₆₋₁₀ aryl, or 5-10 membered heteroaryl wherein the alkyl, alkenyl, alkynyl, cycloalkyl, heterocyclyl, aryl, or 5-10 membered heteroaryl is each optionally substituted with one to four Z^{1b}; and
each R^{3e} is independently H, C₁₋₆ alkyl, C₁₋₆ haloalkyl, —C₁₋₄ alkyl-N(R^{9a})(R^{9b}), —C₁₋₄ alkyl-C(O)N(R^{9a})(R^{9b}), —C₁₋₄ alkyl-O—C(O)—O—C₁₋₄ alkyl, —C₁₋₄ alkyl-O—C(O)—C₁₋₄ alkyl-N(R^{9a})(R^{9b}), —C₁₋₄ alkyl-C₃₋₈ cycloalkyl, —C₁₋₄ alkyl-heterocyclyl, C₃₋₁₀ cycloalkyl, heterocyclyl, C₆₋₁₀ aryl, or 5-10 membered heteroaryl, wherein the alkyl, alkenyl, cycloalkyl, heterocyclyl, aryl, or 5-10 membered heteroaryl in each instance is optionally substituted with one to four Z^{1b}; wherein each 5-10 membered heteroaryl has 5 to 12 ring members and has one to four heteroatoms, each is independently N, O, or S; and wherein each heterocyclyl has 3 to 12 ring members and has one to four heteroatoms, each is independently N, O, or S.

In some embodiments of the compound of Formula I-A-2, I-A-3, I-A-5, I-A-6, I-A-7, or a pharmaceutically acceptable salt thereof, each R^{5a} and R^{5b} is independently H, C₁₋₉ alkyl, or C₂₋₆ alkoxyalkyl, wherein the alkyl or alkoxyalkyl is each optionally substituted with one or more halogens.

In some embodiments of the compound of Formula I-A-2, I-A-3, I-A-4, I-A-5, I-A-6, I-A-7, or pharmaceutically acceptable salt thereof, ring C is a C₆₋₁₀ aryl or 5-10 membered heteroaryl selected from the structures shown below:

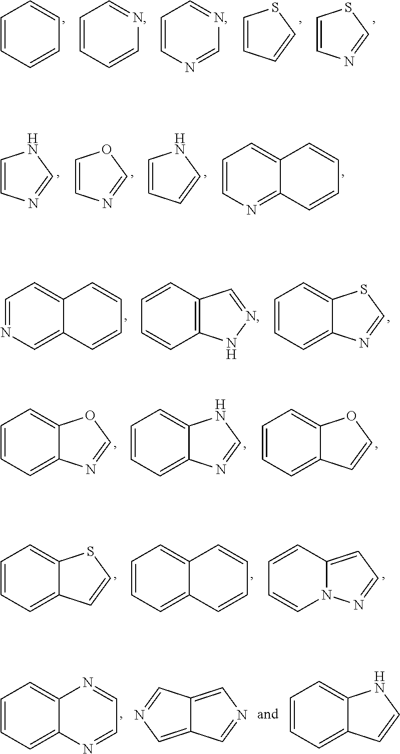

which is each optionally substituted with one to four substituents selected from the group consisting of H, OH, oxo, halogen, C₁₋₆ alkyl, C₁₋₆ haloalkyl, C₁₋₆ alkoxy, C₁₋₆ haloalkoxy, C₂₋₆ alkoxyalkyl, C₂₋₆ alkenyl, C₂₋₆ alkynyl, C₃₋₁₀ cycloalkyl, heterocyclyl, —NO₂, —N₃, —CN, —O—C₁₋₆ alkyl, —C(O)—C₁₋₆ alkyl, —C(O)—N(C₀₋₆ alkyl)(C₀₋₆ alkyl), and —N(C₀₋₆ alkyl)(C₀₋₆ alkyl), wherein each of the one to four substituents can be optionally substituted with one or more halogens.

In some embodiments of the compound of Formula I-A-1,

or pharmaceutically acceptable salt thereof, Ring A is one of

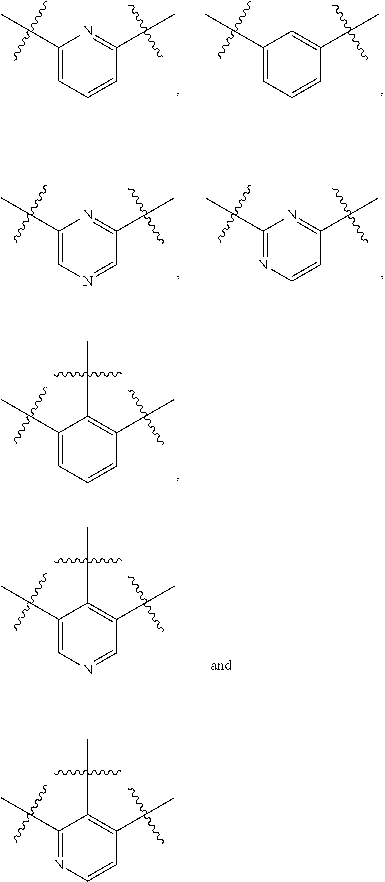

which is each optionally substituted with one to four Z^{1a}.

In some embodiments of the compound of Formula I-A-1, I-A-2, I-A-3, I-A-4, I-A-5, I-A-6, I-A-7, or pharmaceutically acceptable salt thereof, Z^{1a} is independently H, halogen, C₁₋₉ alkyl, C₁₋₆ alkoxy, C₂₋₆ alkoxyalkyl, C₂₋₆ alkenyl, C₂₋₆ alkynyl, C₃₋₁₅ cycloalkyl, or heterocyclyl, which is each optionally substituted with one or more halogens.

In some embodiments of the compound of Formula I-A-1, I-A-2, I-A-3, I-A-4, I-A-5, I-A-6, I-A-7, or pharmaceutically acceptable salt thereof, wherein R² is C₁₋₆ alkyl, —C₁₋₆ alkyl-alkoxy, —C₁₋₆ alkyl-cycloalkoxy, C₁₋₆ haloalkyl, C₂₋₆ alkenyl, C₂₋₆ alkynyl, C₃₋₁₀ cycloalkyl, heterocyclyl, C₆₋₁₀ aryl, or 5-10 membered heteroaryl, which is each optionally substituted with one to four Z¹. Preferably, R² is one of

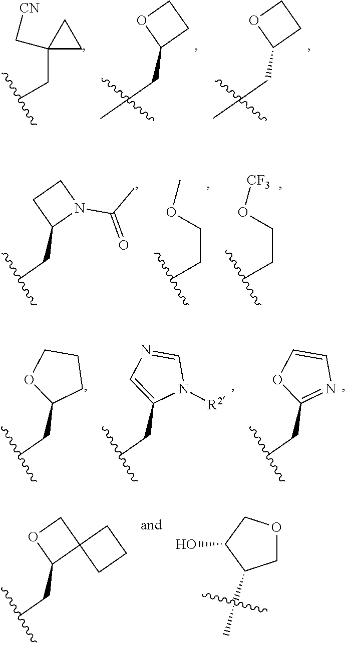

which is each optionally substituted with one to four Z¹.

In some embodiments of the compound of Formula I-A-1, I-A-2, I-A-3, I-A-4, I-A-5, I-A-6, I-A-7, or pharmaceutically acceptable salt thereof, R^{2′} is H, C₁₋₆ alkyl, C₁₋₆ haloalkyl, —C₁₋₆ alkyl or -cycloalkoxy, which is each optionally substituted with one or more halogens.

In some embodiments of the compound of of Formula I-A-1, I-A-2, I-A-3, I-A-4, I-A-5, I-A-6, I-A-7, or pharmaceutically acceptable salt thereof, each R³ and R^{3′} is independently H, C₆₋₁₀ aryl, 5-10 membered heteroaryl, —C(O)R^{3a}, —CH₂C(O)OR^{3a}, —C(O)N(R^{3a})(R^{3b}), —N(R^{3a})C(O)R^{3b}, —N(R^{3a})C(O)OR^{3b}, —N(R^{3a})C(O)N(R^{3b})₂, —C(O)NHS(O)₂R^{3a}, —C(O)NR^{3a}S(O)₂R^{3b}, —C(O)NR^{3a}S(O)₂NR^{3b}R^{3c}, —C(O)NR^{3a}—S(O)(═NR^{3b})R^{3c}, —S(O)₂N(R^{3a})(R^{3b}), —N(R^{3a})S(O)₂R^{3b}, —S(O)₂NHC(O)R^{3a}, or —O—C₁₋₆alkyl-C(O)OR^{3a}, which is each optionally substituted with one to four R^{3d}.

Preferably, each R³ and R^{3′} is independently H, C(O)NR^{3a}(CH₂)ₘR^{3b}, C(O)NR^{3a}(CH₂)ₘC(O)R^{3b}, NR^{3a} C(O) (CH₂)ₘR^{3b}, NR^{3a} (CH₂)ₘ R^{3b}, NR^{3a} (CH₂)ₘ C(O)R^{3b}, C(O)NR^{3a}R^{3b}, or NR^{3a}C(O) R^{3b}, which is each optionally substituted with one to four R^{3d}, wherein m=0, 1, 2, or 3. Preferably, each R³ and R^{3′} is independently H, C(O)NHR^{3b}, or NHC(O) R^{3b}.

In some embodiments of the compound of of Formula I-A-1, I-A-2, I-A-3, I-A-4, I-A-5, I-A-6, I-A-7, or pharmaceutically acceptable salt thereof, R^{3b} is independently H, C₁₋₆ alkyl, C₁₋₆ haloalkyl, C₂₋₈ alkoxyalkyl, —C₁₋₄ alkyl-N(R^{9a})(R^{9b}), —C₁₋₄ alkyl-C(O)N(R^{9a})(R^{9b}), —C₁₋₄ alkyl-O—C(O)—O—C₁₋₄ alkyl, —C₁₋₄ alkyl-O—C(O)—C₁₋₄ alkyl-N(R^{9a})(R^{9b}), —C₁₋₄ alkyl-C₃₋₈ cycloalkyl, —C₁₋₄ alkyl-heterocyclyl, C₂₋₆ alkenyl, C₂₋₆ alkynyl, C₃₋₁₀ cycloalkyl, heterocyclyl, C₆₋₁₀ aryl, 5-10 membered heteroaryl, —CH₂CH(N(R^{9a})₂)C(O)OR^{9b}, C₁₋₆ alkyl-heterocyclyl, C₁₋₆ alkyl-aryl, or C₁₋₆ alkyl-(5-10 membered) heteroaryl, wherein the alkyl, alkenyl, cycloalkyl, heterocyclyl, aryl, or 5-10 membered heteroaryl in each instance is optionally substituted with one to four Z^{1b}.

Preferably, R^{3b} is C₅₋₁₀ substituted aryl or substituted 5-10 membered heteroaryl, which is each optionally substituted with one to five R^{3f}, wherein each R^{3f} is independently H, OH, oxo, NH₂, halogen, —NO₂, —N₃, —CN, C₁₋₆ alkyl, C₁₋₆ haloalkyl, C₁₋₆ alkoxy, C₁₋₆ haloalkoxy, C₂₋₆ alkoxyalkyl, C₂₋₆ alkenyl, C₂₋₆ alkynyl, C₃₋₁₀ cycloalkyl, heterocyclyl, —O—C₁₋₆ alkyl, —C(O)—C₁₋₆ alkyl, —C(O)—N(R^{12a})(R^{12b}), —N(R^{12a})(R^{12b}), C₆₋₁₀ aryl, or 5-10 membered heteroaryl, and wherein R^{3f} in each instance is optionally substituted with one or more halogens, C₁₋₆ alkyl, C₁₋₆ haloalkyl, C₁₋₆ alkoxy, or C₁₋₆ haloalkoxy.

Preferably, R^{3b} is one of the following:

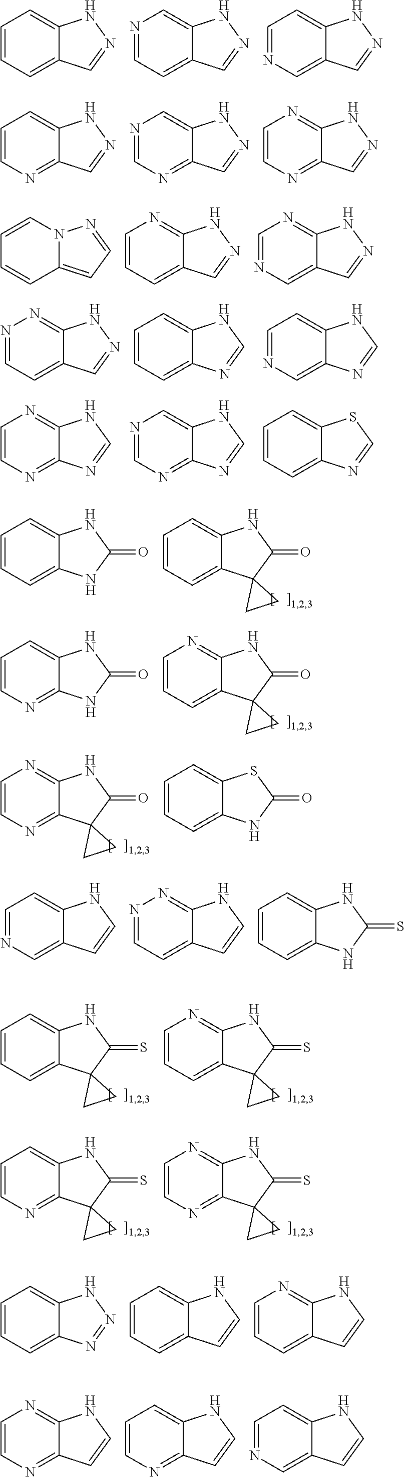

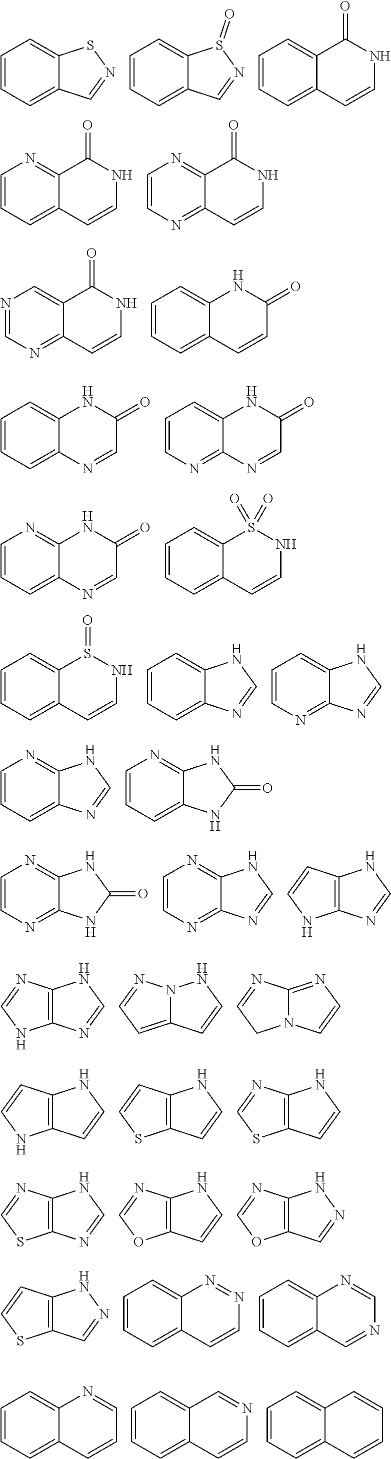

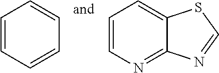 [SMILES: c1ccccc1 ; c1cnc2ncsc2c1]

which is each optionally substituted with one to five R^{3f}.

In some embodiments of the compound of Formula I-A-1, or pharmaceutically acceptable salt thereof, R⁴ is independently H, —OH, C₁₋₉ alkyl, C₁₋₈ haloalkyl, C₁₋₆ haloalkoxy, C₂₋₆ alkoxyalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —NO₂, —CN, —N₃, —O—C₁₋₉ alkyl, or —C(O) C₁₋₉ alkyl, which is each optionally substituted with one to four Z^{1b}; or two R⁴ groups attached to adjacent ring atoms are combined with the atoms to which they are attached to form a C₅₋₁₀ cycloalkyl or heterocyclyl, which is each optionally substituted with one to four Z^{1b}.

Preferably, R⁴ is independently H, halogen, C₁₋₉ alkyl, C₁₋₈ haloalkyl, C₁₋₆ haloalkoxy, C₂₋₆ alkoxyalkyl, C₂₋₆ alkenyl, C₂₋₆ alkynyl, or C₃₋₁₅ cycloalkyl.

In some embodiments, the compound of Formula I-A-1, I-A-2, I-A-3, I-A-4, I-A-5, I-A-6, or I-A-7, or pharmaceutically acceptable salt thereof has one of the structures listed below:

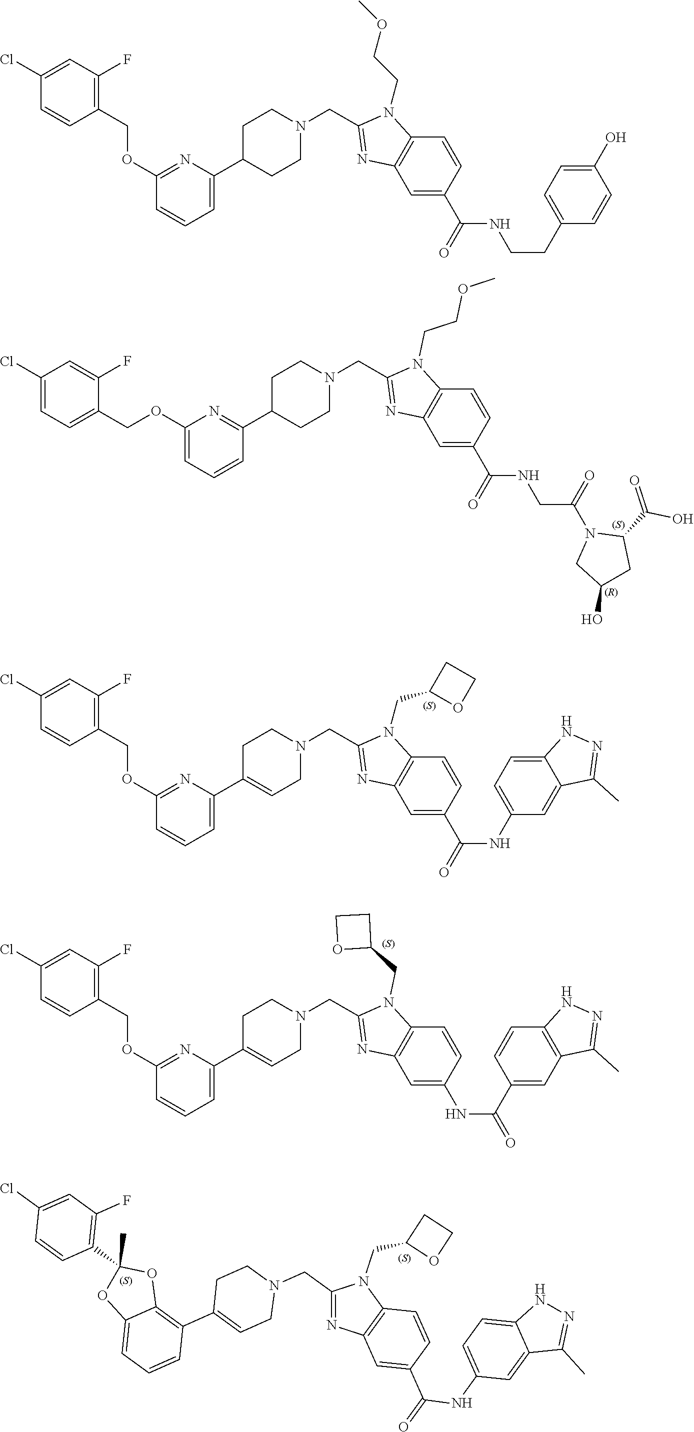

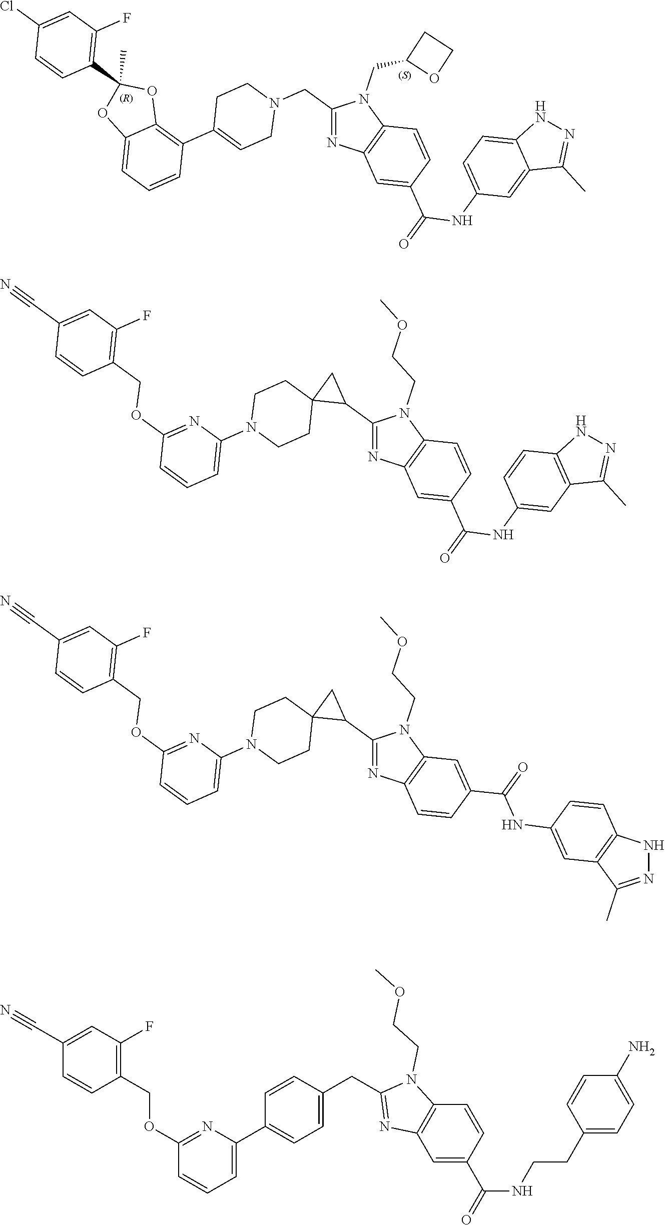

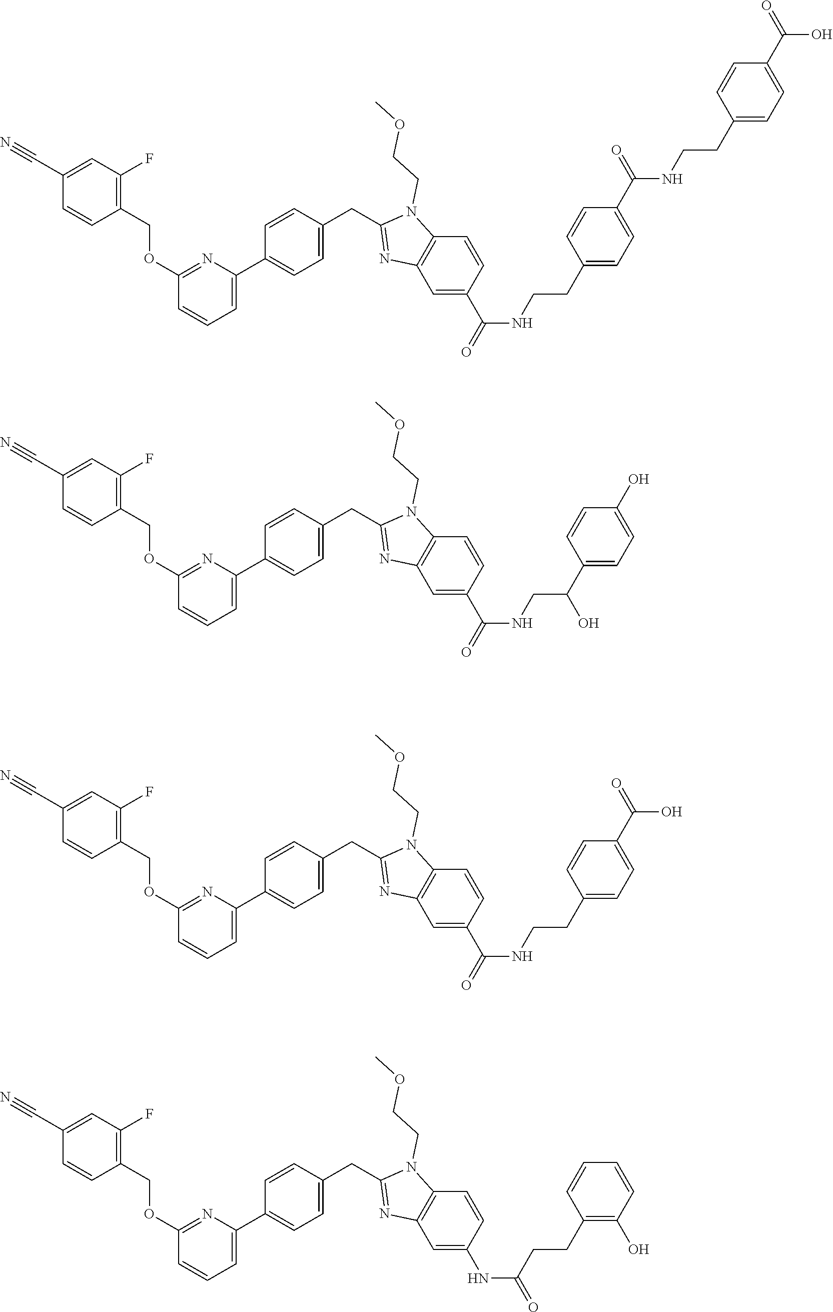 [SMILES: COCCn1c(Cc2ccc(-c3cccc(OCc4ccc(C#N)cc4F)n3)cc2)nc2cc(C(=O)NCCc3ccc(C(=O)NCCc4ccc(C(=O)O)cc4)cc3)ccc21 ; COCCn1c(Cc2ccc(-c3cccc(OCc4ccc(C#N)cc4F)n3)cc2)nc2cc(C(=O)NCC(O)c3ccc(O)cc3)ccc21 ; COCCn1c(Cc2ccc(-c3cccc(OCc4ccc(C#N)cc4F)n3)cc2)nc2cc(C(=O)NCCc3ccc(C(=O)O)cc3)ccc21 ; COCCn1c(Cc2ccc(-c3cccc(OCc4ccc(C#N)cc4F)n3)cc2)nc2cc(NC(=O)CCc3ccccc3O)ccc21]

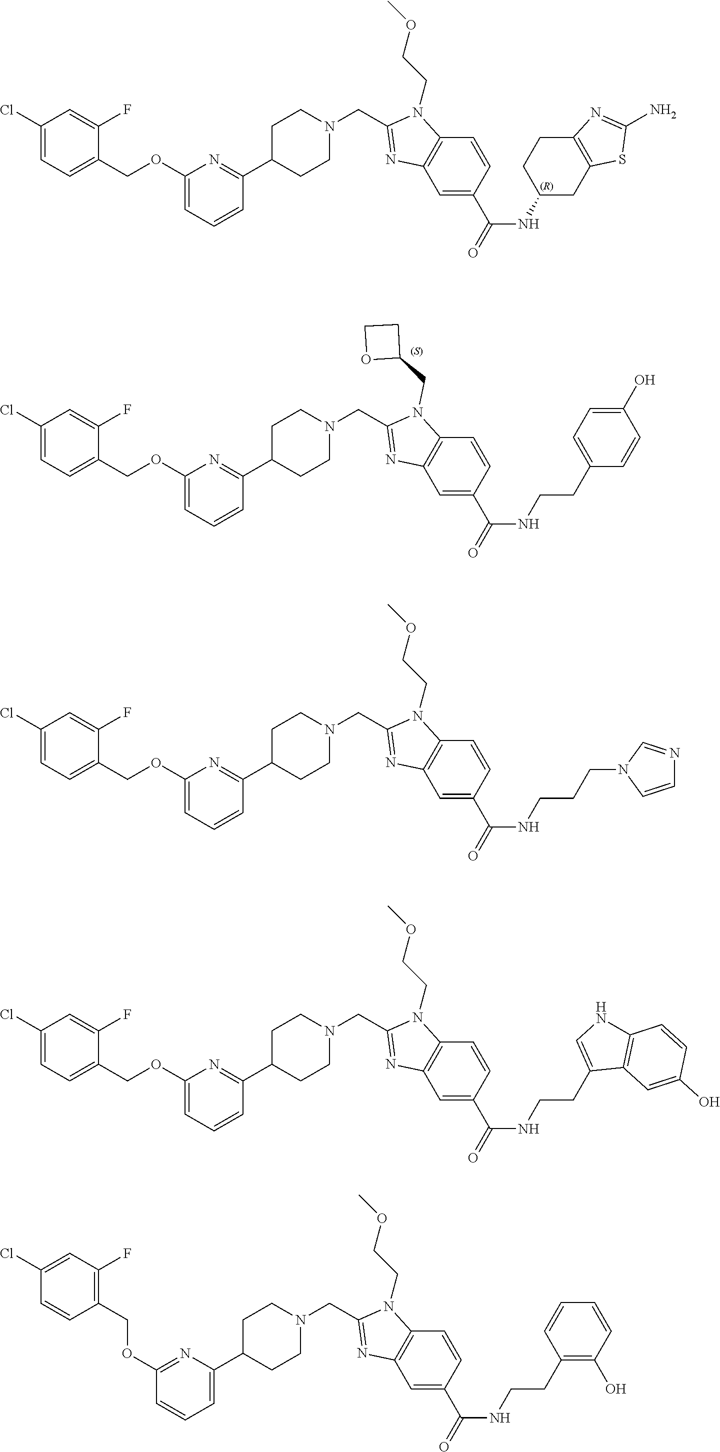

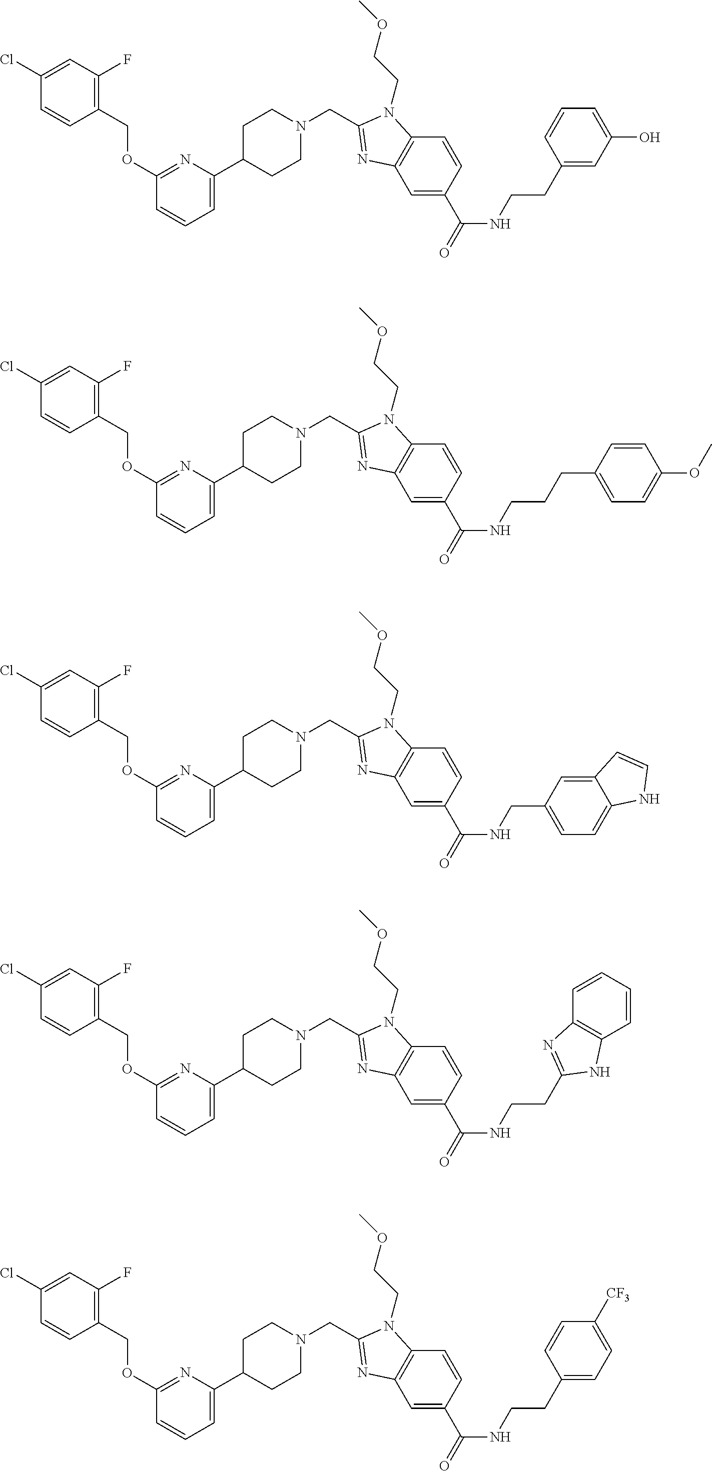 [SMILES: COCCn1c(CN2CCC(c3cccc(OCc4ccc(Cl)cc4F)n3)CC2)nc2cc(C(=O)NCCc3cccc(O)c3)ccc21 ; COCCn1c(CN2CCC(c3cccc(OCc4ccc(Cl)cc4F)n3)CC2)nc2cc(C(=O)NCCCc3ccc(OC)cc3)ccc21 ; COCCn1c(CN2CCC(c3cccc(OCc4ccc(Cl)cc4F)n3)CC2)nc2cc(C(=O)NCc3ccc4[nH]ccc4c3)ccc21 ; COCCn1c(CN2CCC(c3cccc(OCc4ccc(Cl)cc4F)n3)CC2)nc2cc(C(=O)NCCc3nc4ccccc4[nH]3)ccc21 ; COCCn1c(CN2CCC(c3cccc(OCc4ccc(Cl)cc4F)n3)CC2)nc2cc(C(=O)NCCc3ccc(C(F)(F)F)cc3)ccc21]

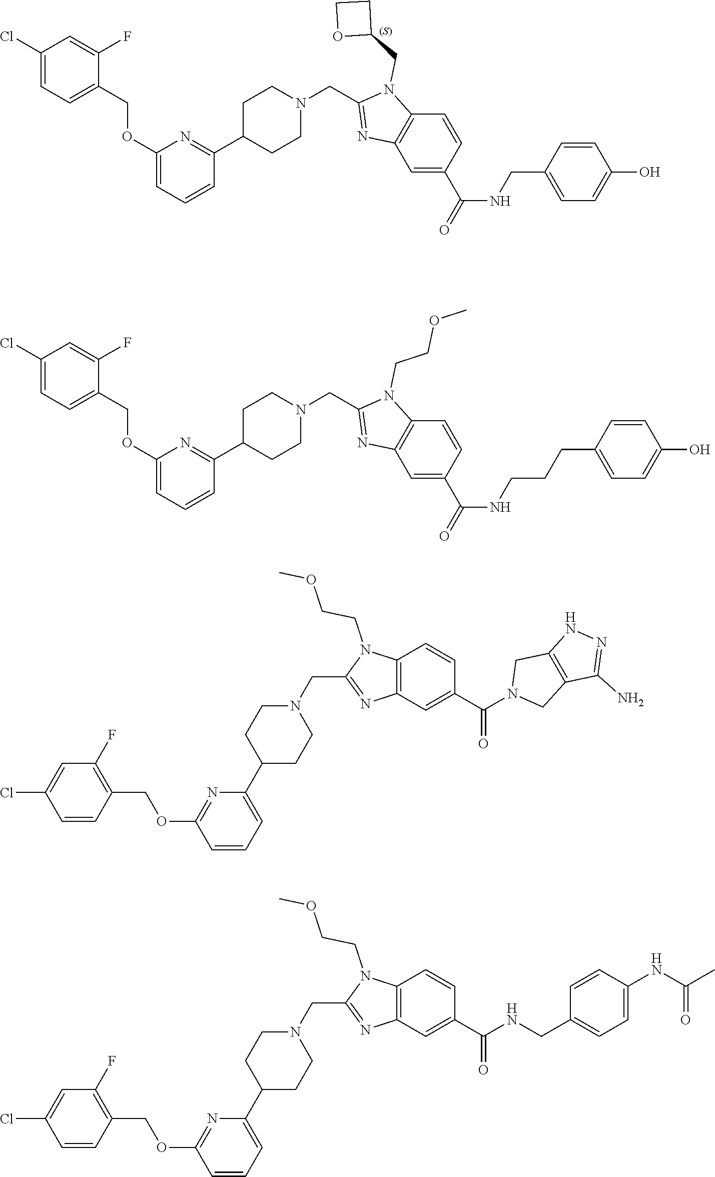

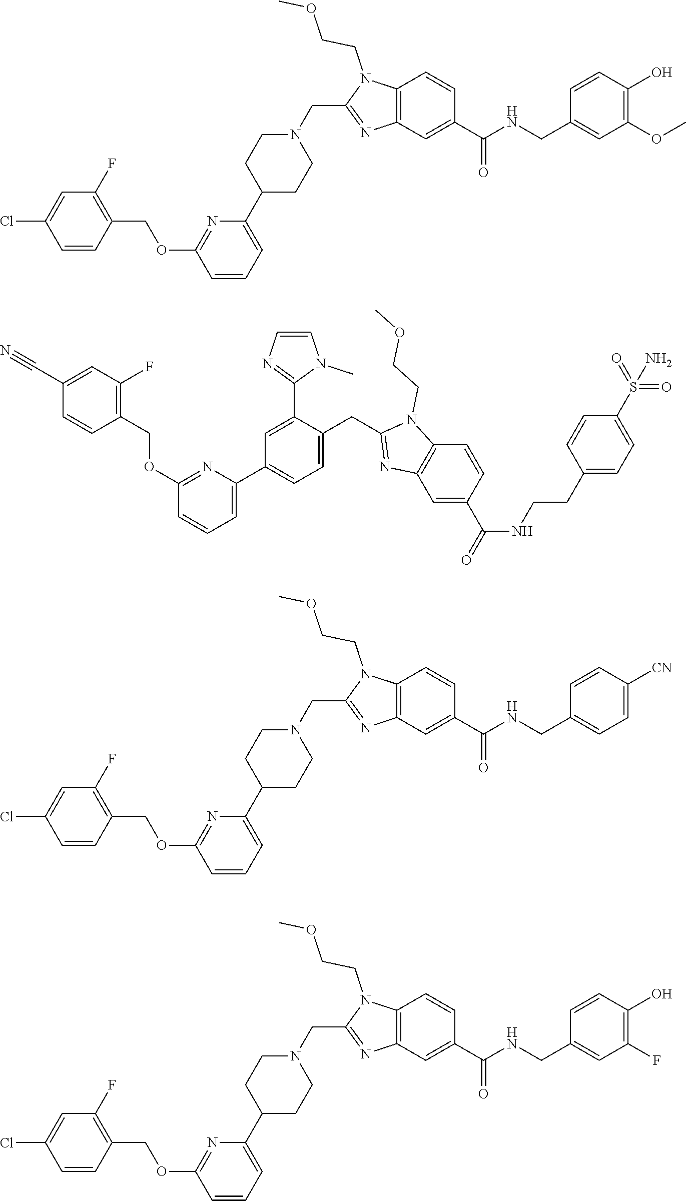 [SMILES: COCCn1c(CN2CCC(c3cccc(OCc4ccc(Cl)cc4F)n3)CC2)nc2cc(C(=O)NCc3ccc(O)c(OC)c3)ccc21 ; COCCn1c(Cc2ccc(-c3cccc(OCc4ccc(C#N)cc4F)n3)cc2-c2nccn2C)nc2cc(C(=O)NCCc3ccc(S(N)(=O)=O)cc3)ccc21 ; COCCn1c(CN2CCC(c3cccc(OCc4ccc(Cl)cc4F)n3)CC2)nc2cc(C(=O)NCc3ccc(C#N)cc3)ccc21 ; COCCn1c(CN2CCC(c3cccc(OCc4ccc(Cl)cc4F)n3)CC2)nc2cc(C(=O)NCc3ccc(O)c(F)c3)ccc21]

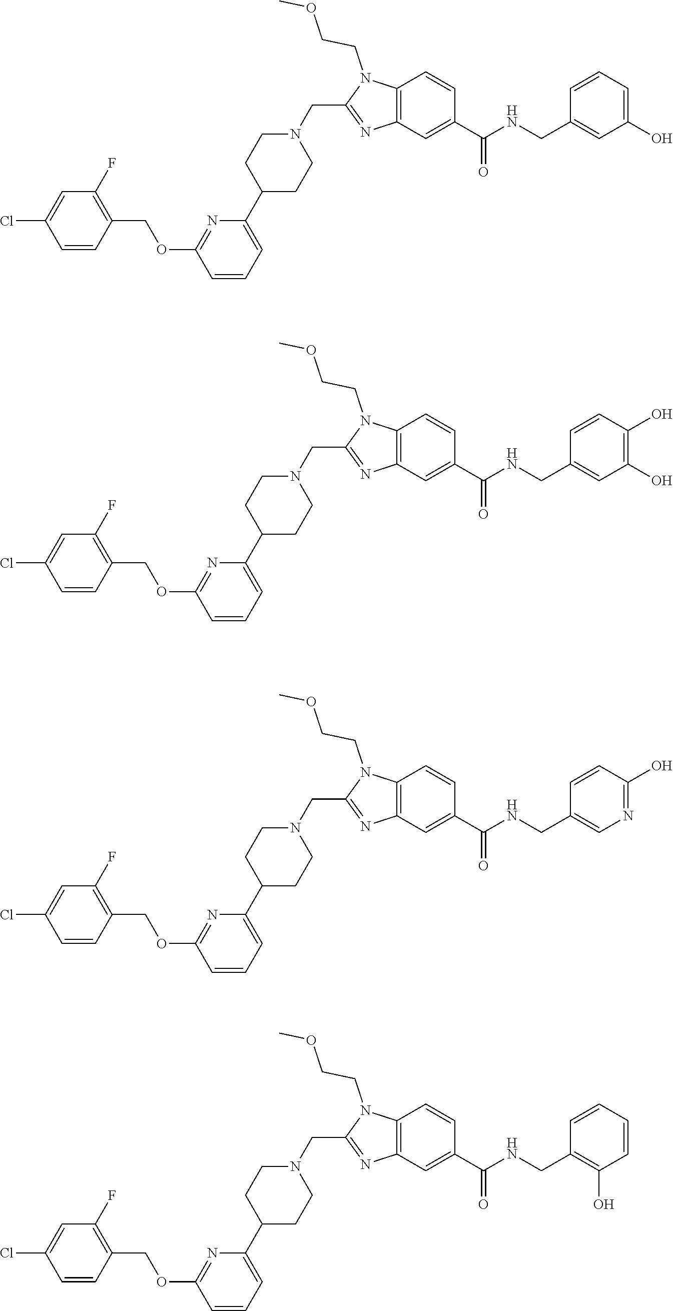 [SMILES: COCCn1c(CN2CCC(c3cccc(OCc4ccc(Cl)cc4F)n3)CC2)nc2cc(C(=O)NCc3cccc(O)c3)ccc21 ; COCCn1c(CN2CCC(c3cccc(OCc4ccc(Cl)cc4F)n3)CC2)nc2cc(C(=O)NCc3ccc(O)c(O)c3)ccc21 ; COCCn1c(CN2CCC(c3cccc(OCc4ccc(Cl)cc4F)n3)CC2)nc2cc(C(=O)NCc3ccc(O)nc3)ccc21 ; COCCn1c(CN2CCC(c3cccc(OCc4ccc(Cl)cc4F)n3)CC2)nc2cc(C(=O)NCc3ccccc3O)ccc21]

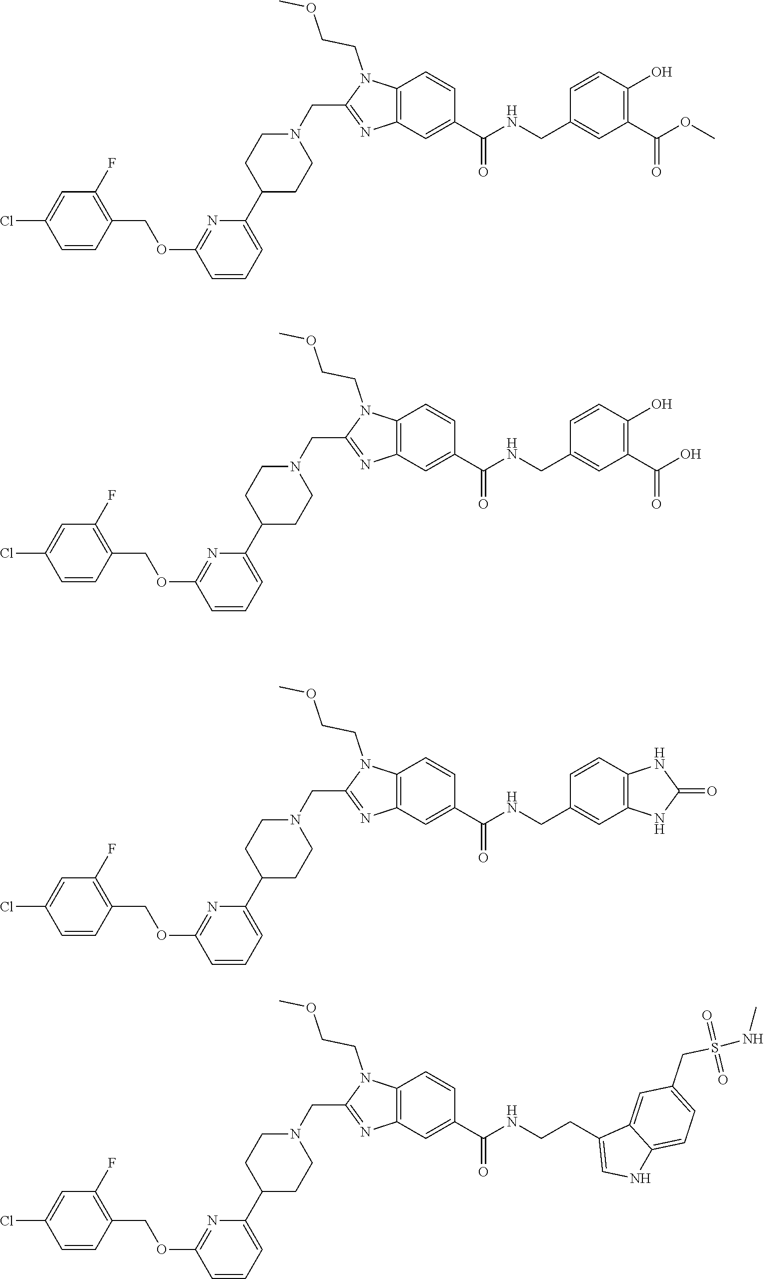 [SMILES: COCCn1c(CN2CCC(c3cccc(OCc4ccc(Cl)cc4F)n3)CC2)nc2cc(C(=O)NCc3ccc(O)c(C(=O)OC)c3)ccc21 ; COCCn1c(CN2CCC(c3cccc(OCc4ccc(Cl)cc4F)n3)CC2)nc2cc(C(=O)NCc3ccc(O)c(C(=O)O)c3)ccc21 ; COCCn1c(CN2CCC(c3cccc(OCc4ccc(Cl)cc4F)n3)CC2)nc2cc(C(=O)NCc3ccc4[nH]c(=O)[nH]c4c3)ccc21 ; CNS(=O)(=O)Cc1ccc2[nH]cc(CCNC(=O)c3ccc4c(c3)nc(CN3CCC(c5cccc(OCc6ccc(Cl)cc6F)n5)CC3)n4CCOC)c2c1]

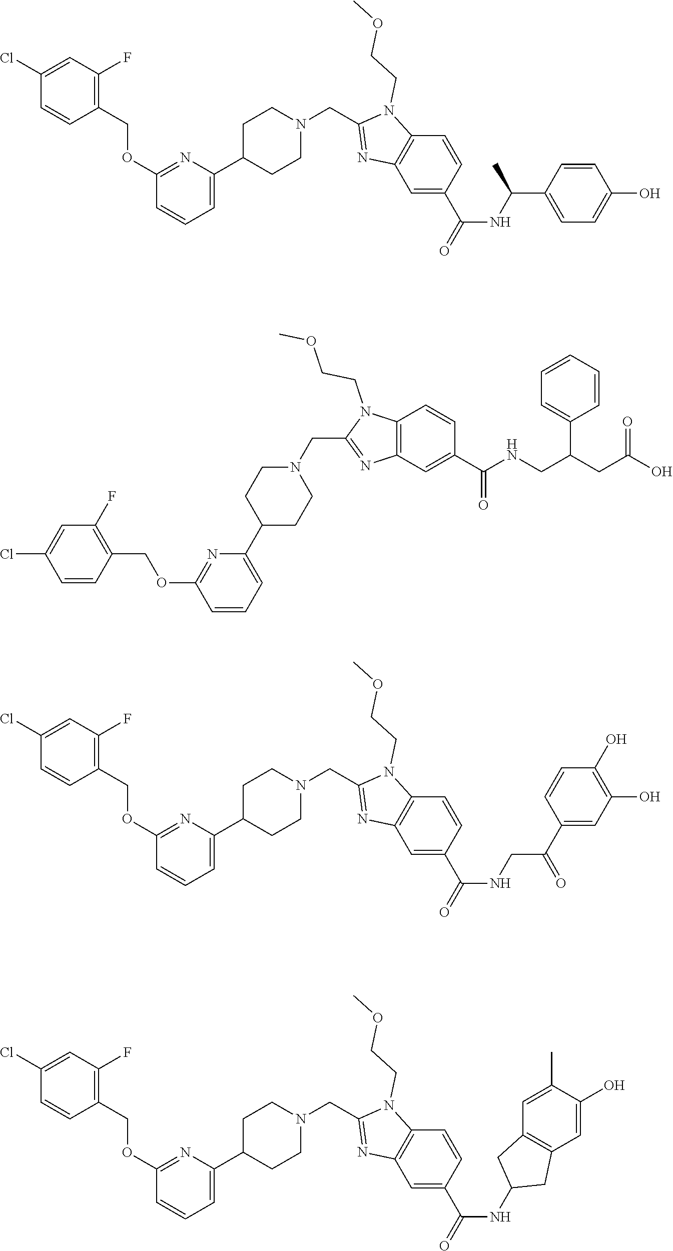 [SMILES: COCCn1c(CN2CCC(c3cccc(OCc4ccc(Cl)cc4F)n3)CC2)nc2cc(C(=O)N[C@@H](C)c3ccc(O)cc3)ccc21 ; COCCn1c(CN2CCC(c3cccc(OCc4ccc(Cl)cc4F)n3)CC2)nc2cc(C(=O)NCC(CC(=O)O)c3ccccc3)ccc21 ; COCCn1c(CN2CCC(c3cccc(OCc4ccc(Cl)cc4F)n3)CC2)nc2cc(C(=O)NCC(=O)c3ccc(O)c(O)c3)ccc21 ; COCCn1c(CN2CCC(c3cccc(OCc4ccc(Cl)cc4F)n3)CC2)nc2cc(C(=O)NC3Cc4cc(C)c(O)cc4C3)ccc21]

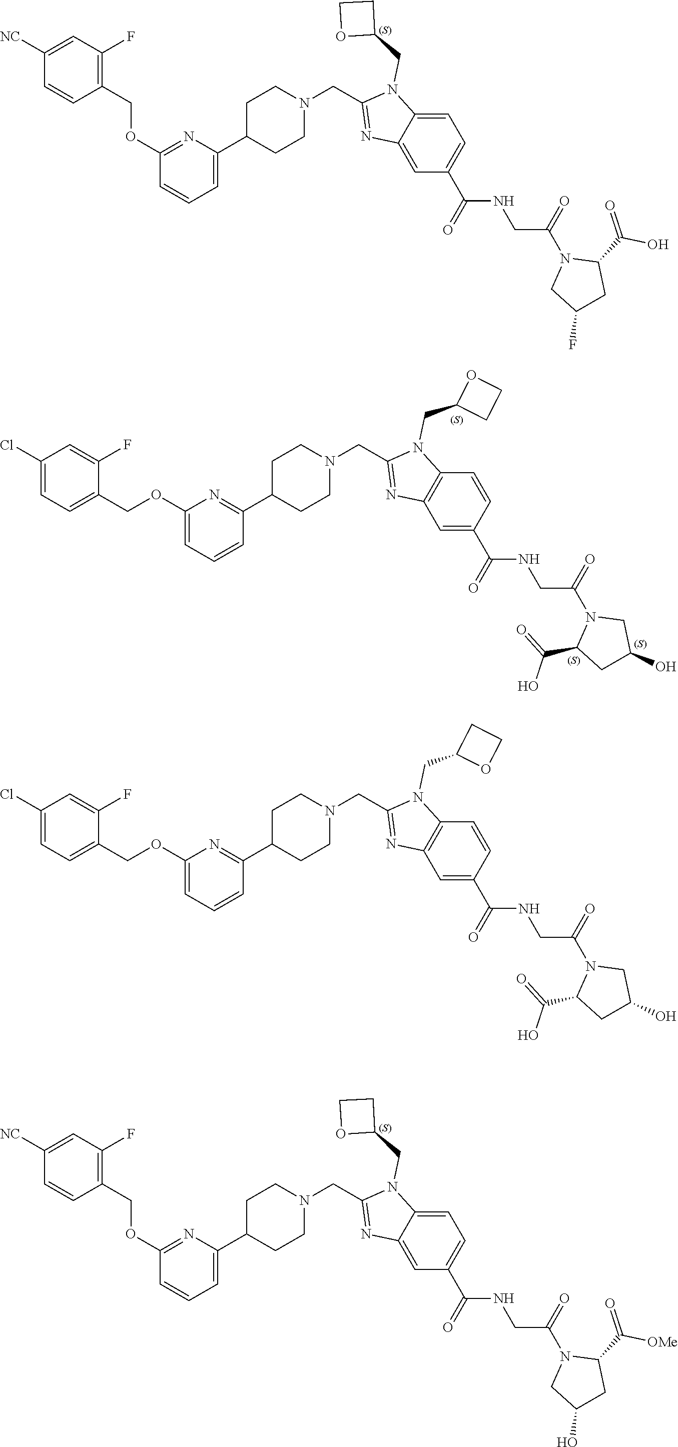

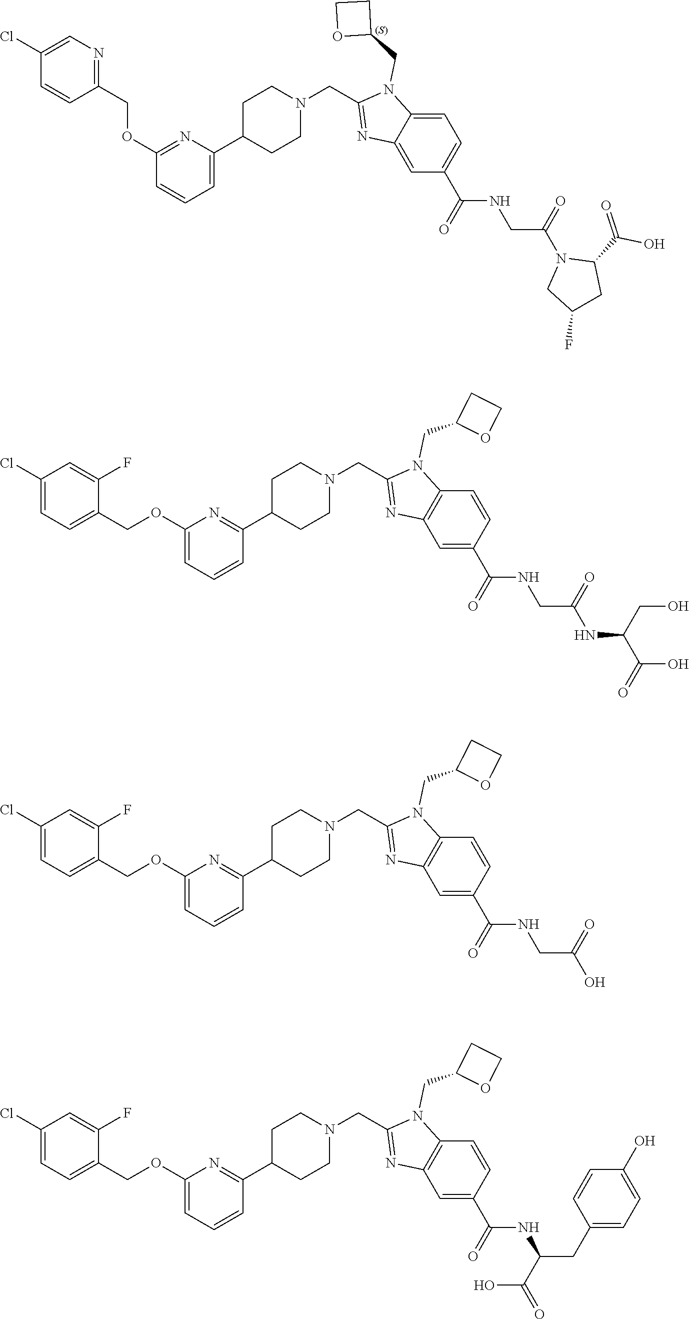

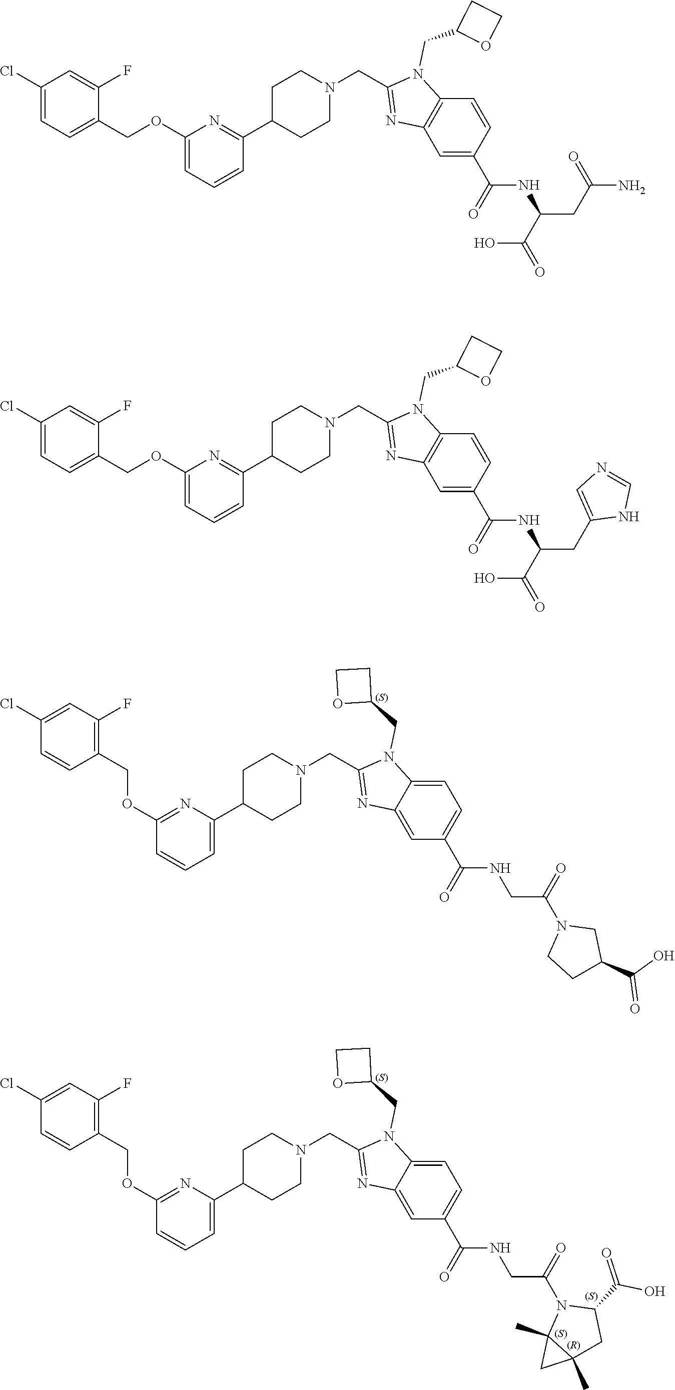

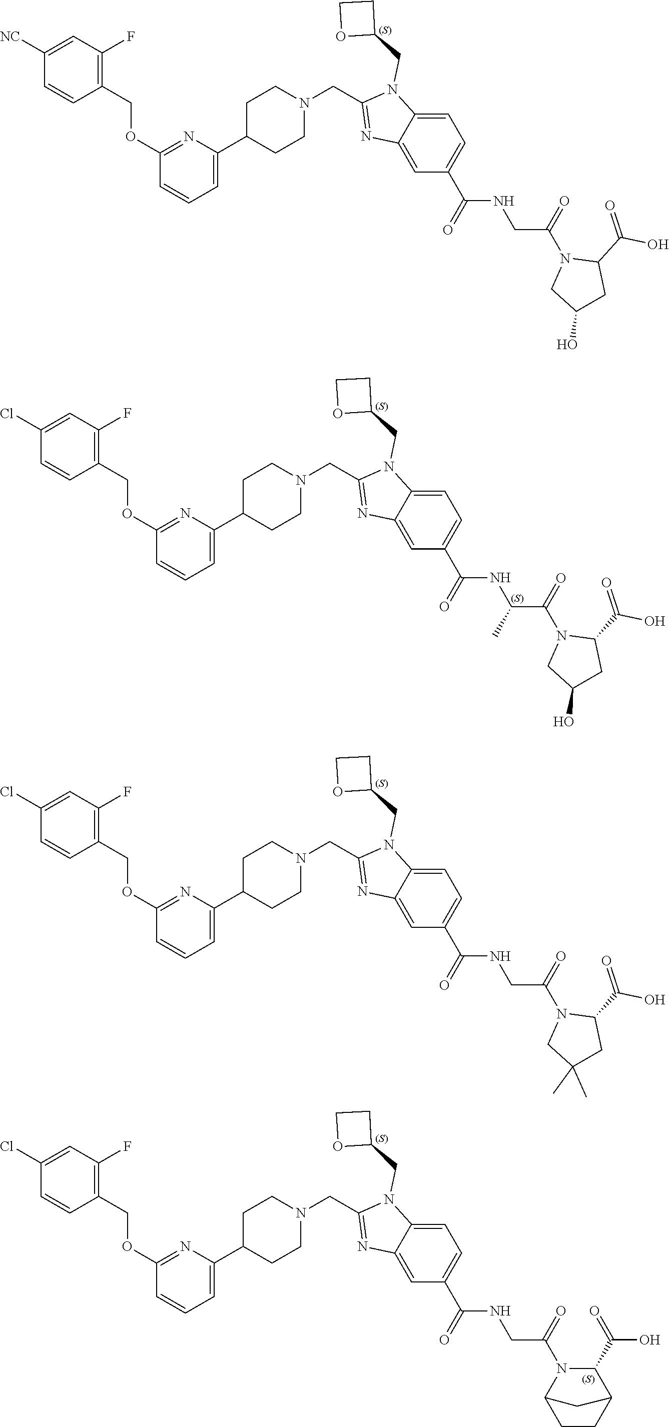

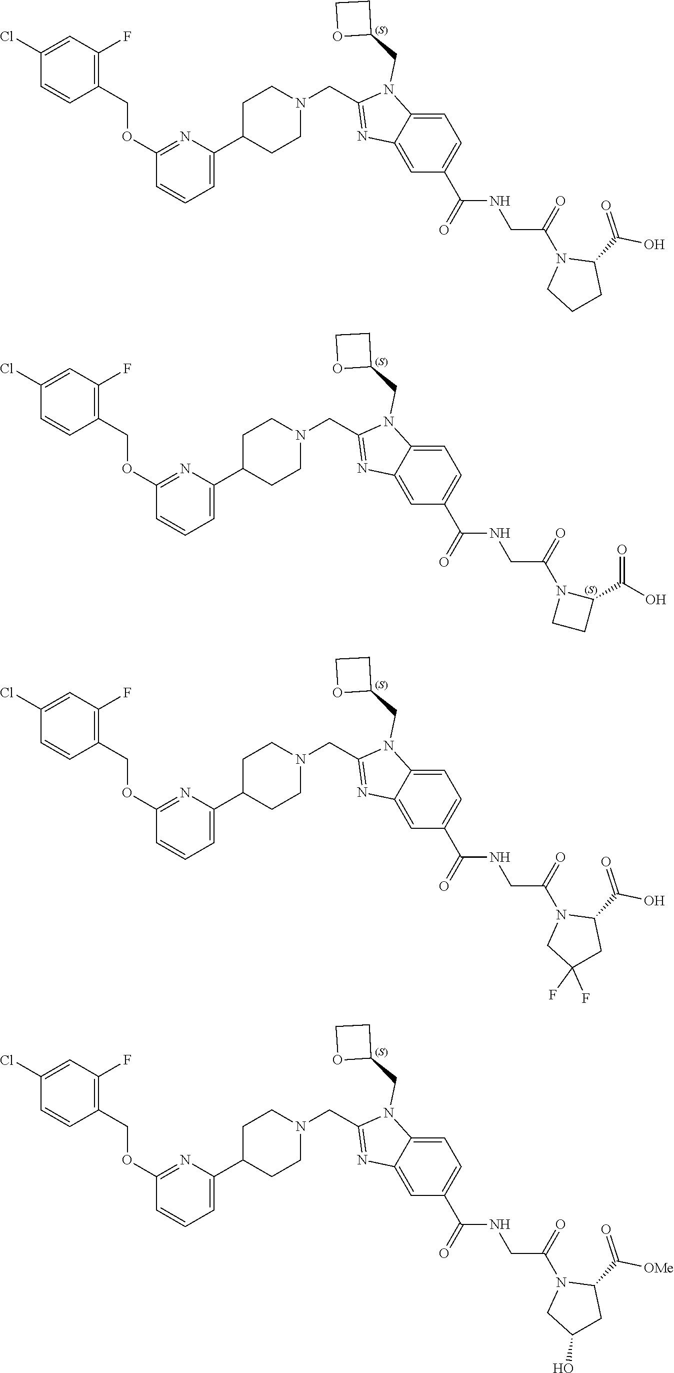

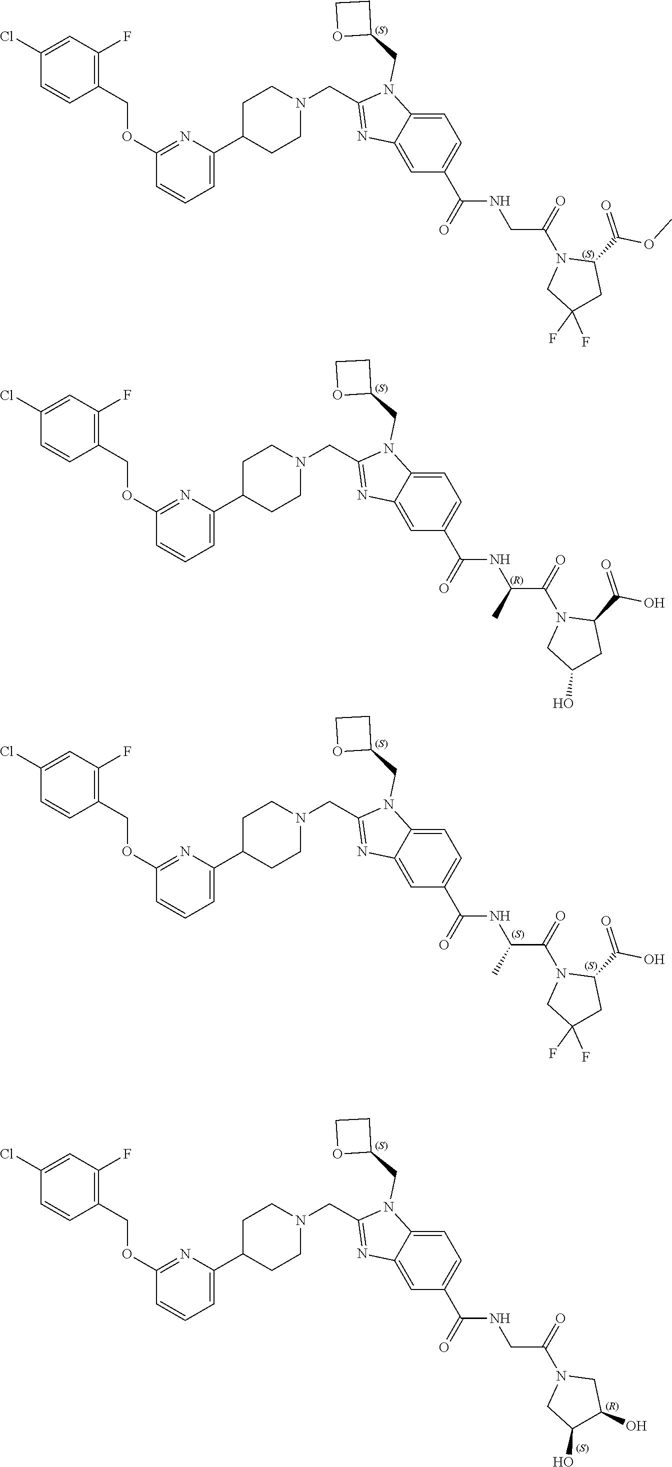

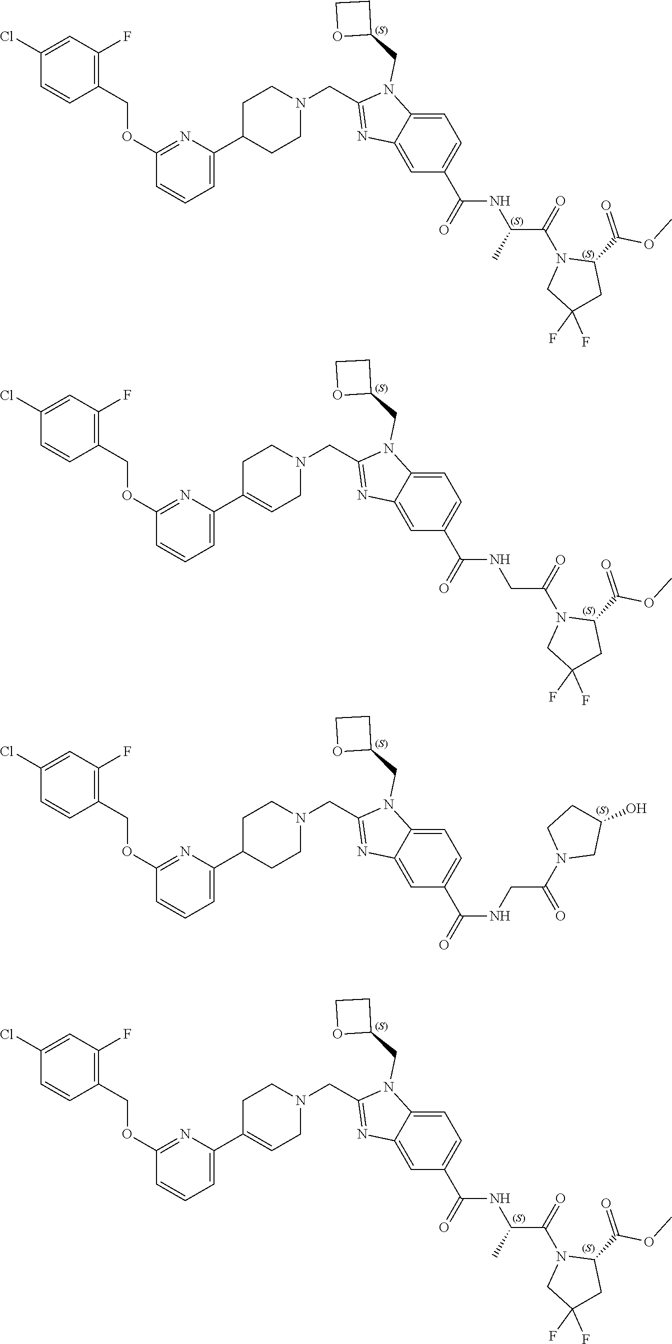

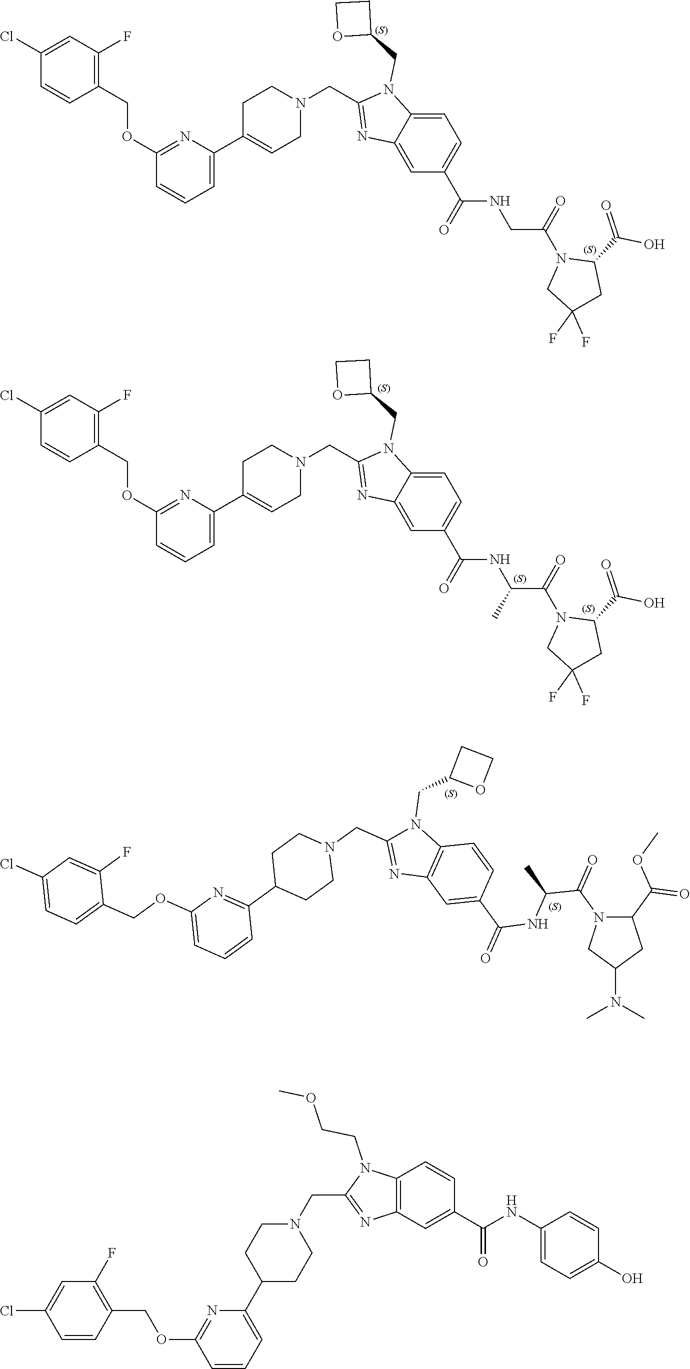

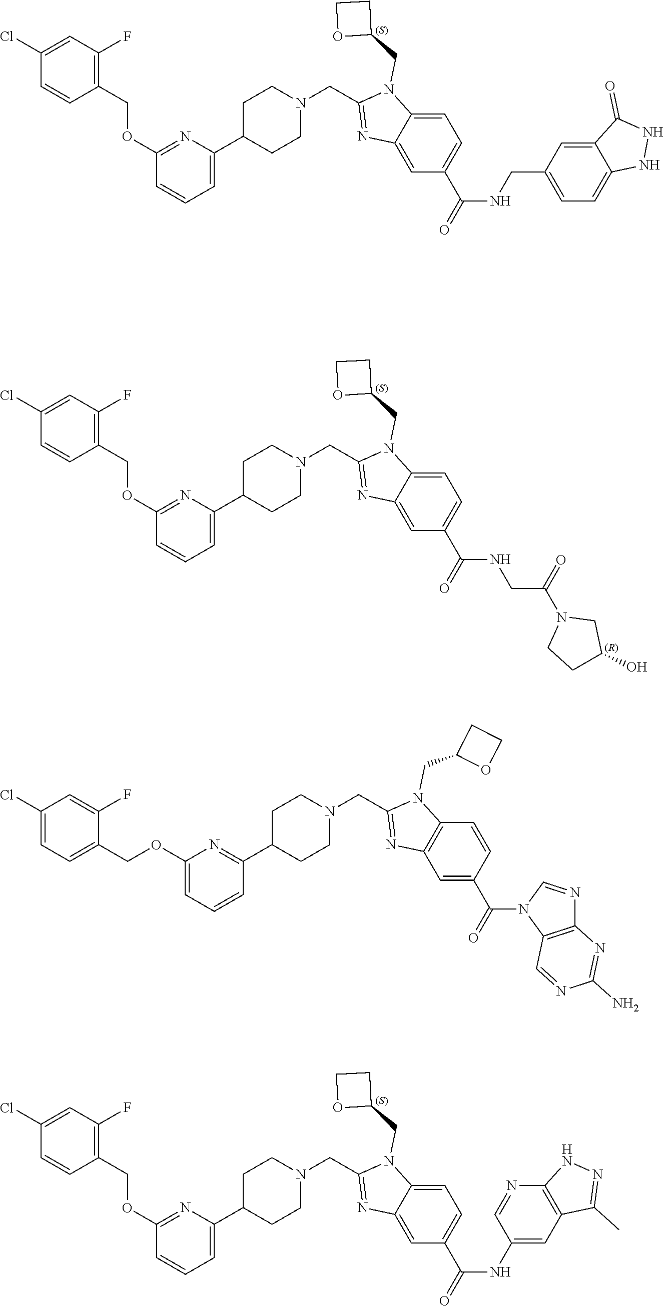

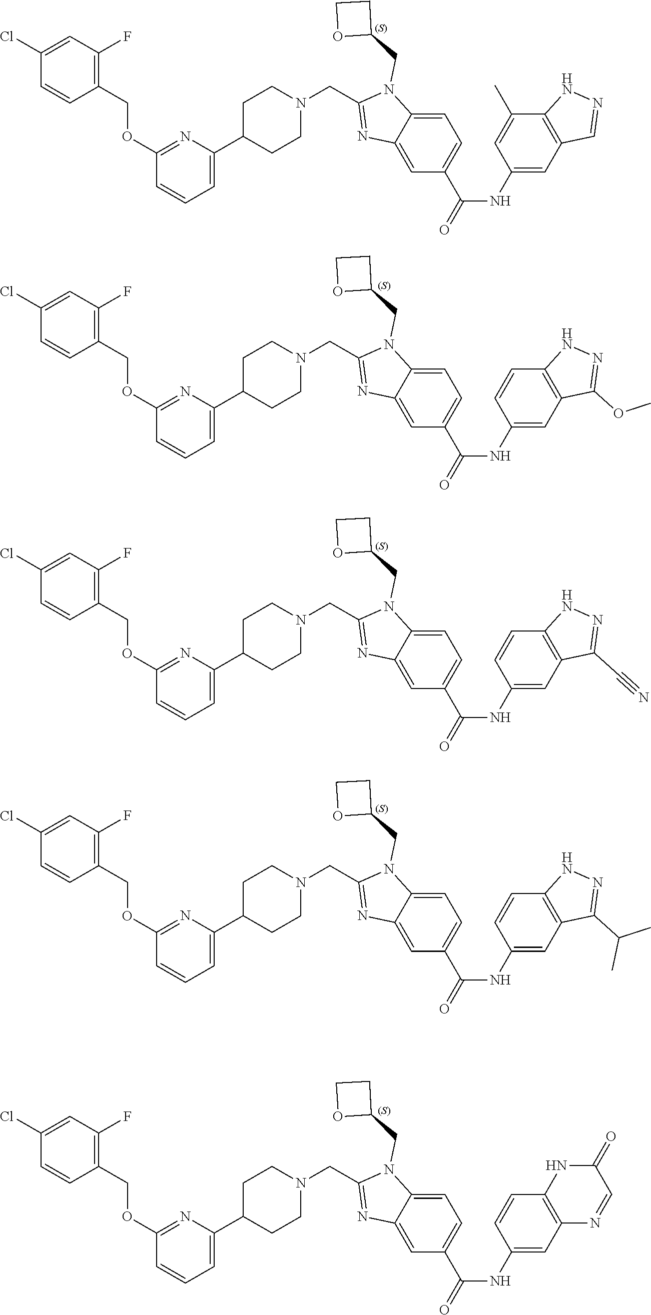

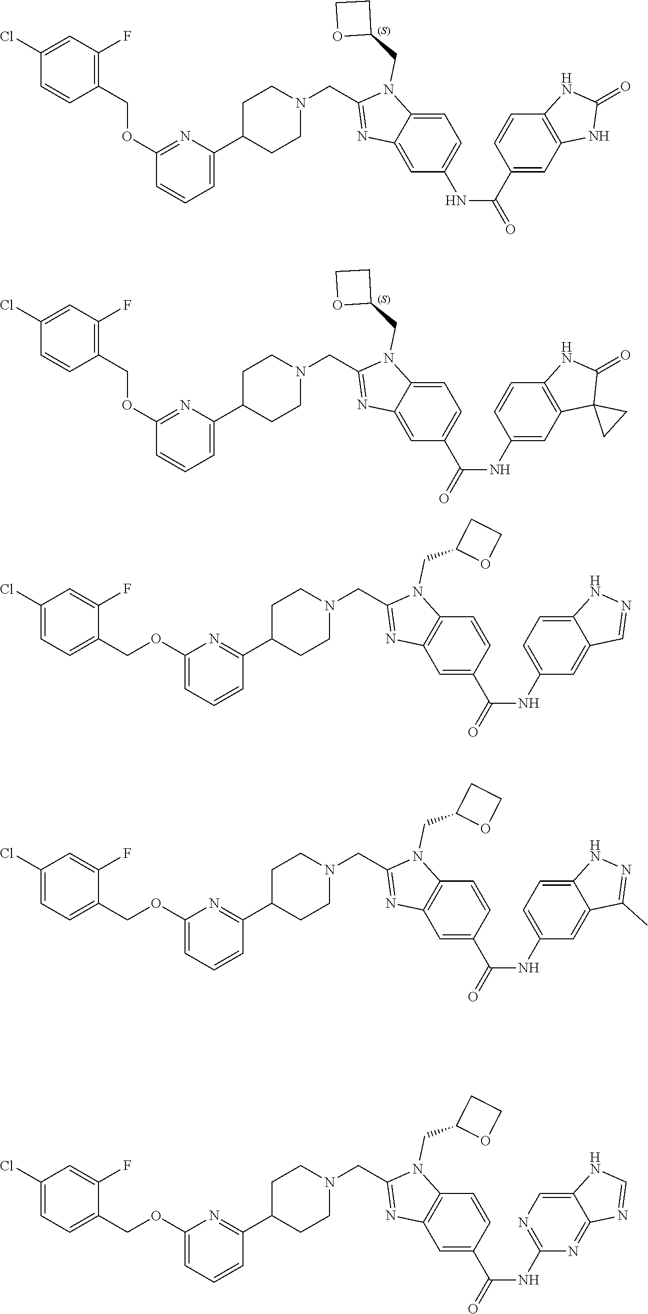

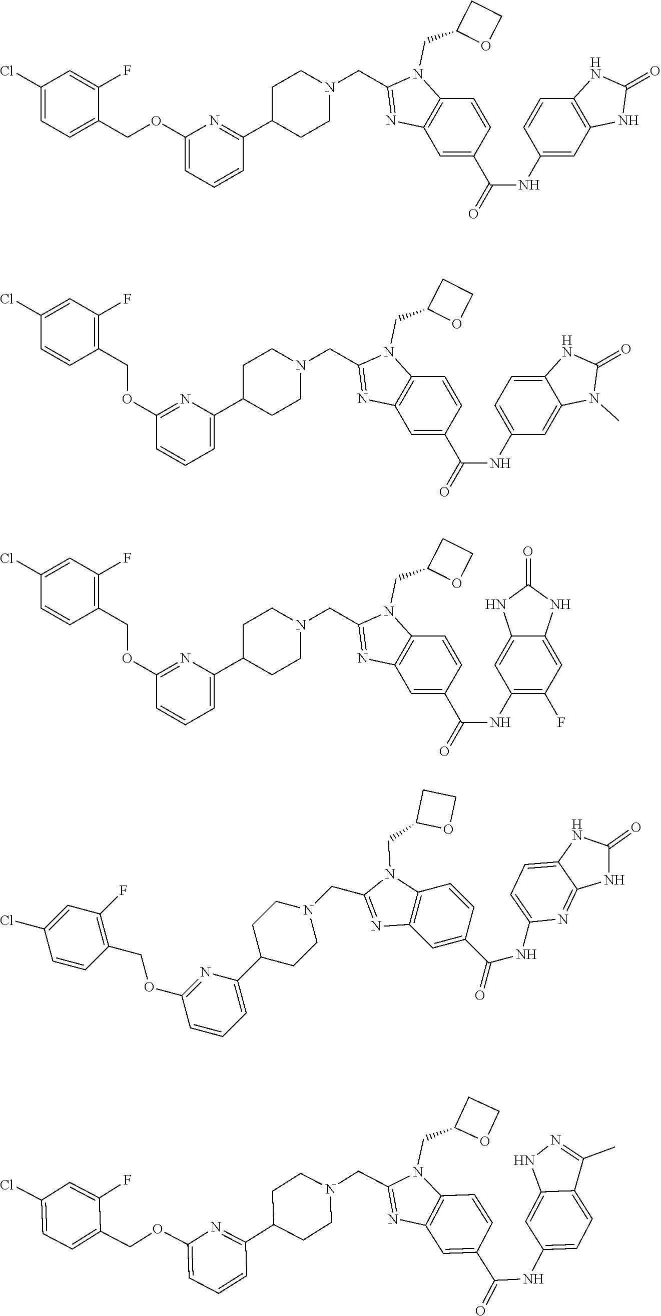

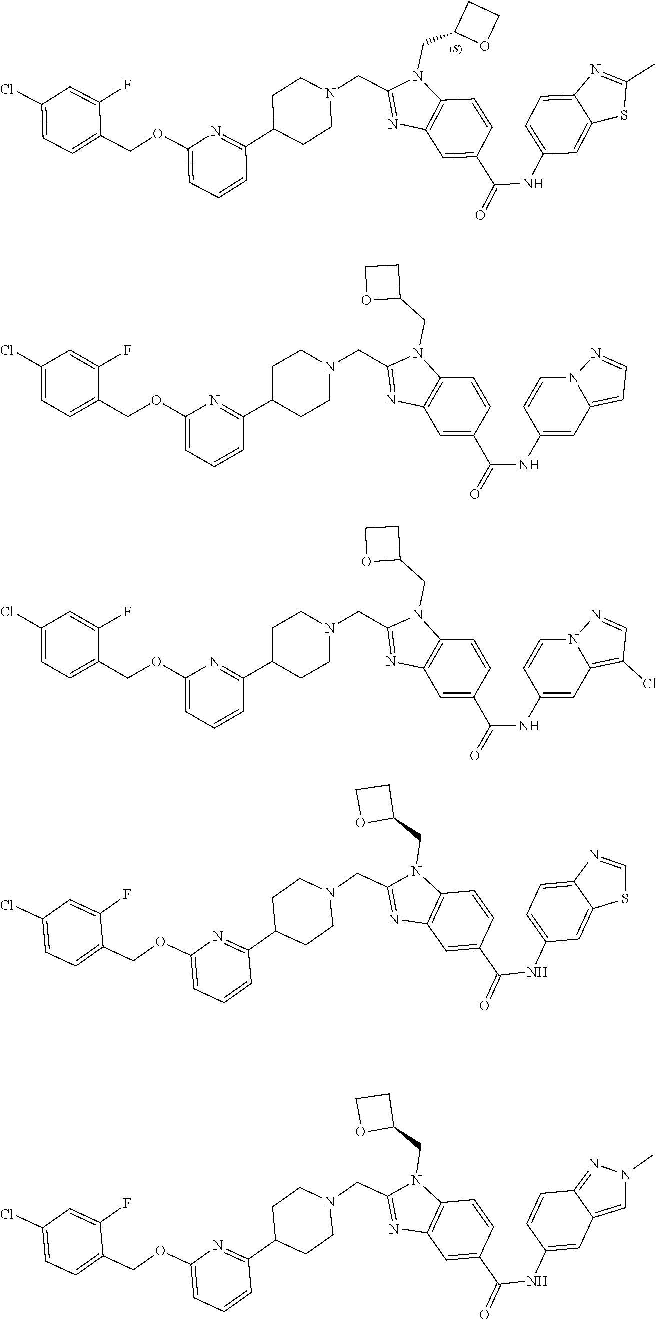

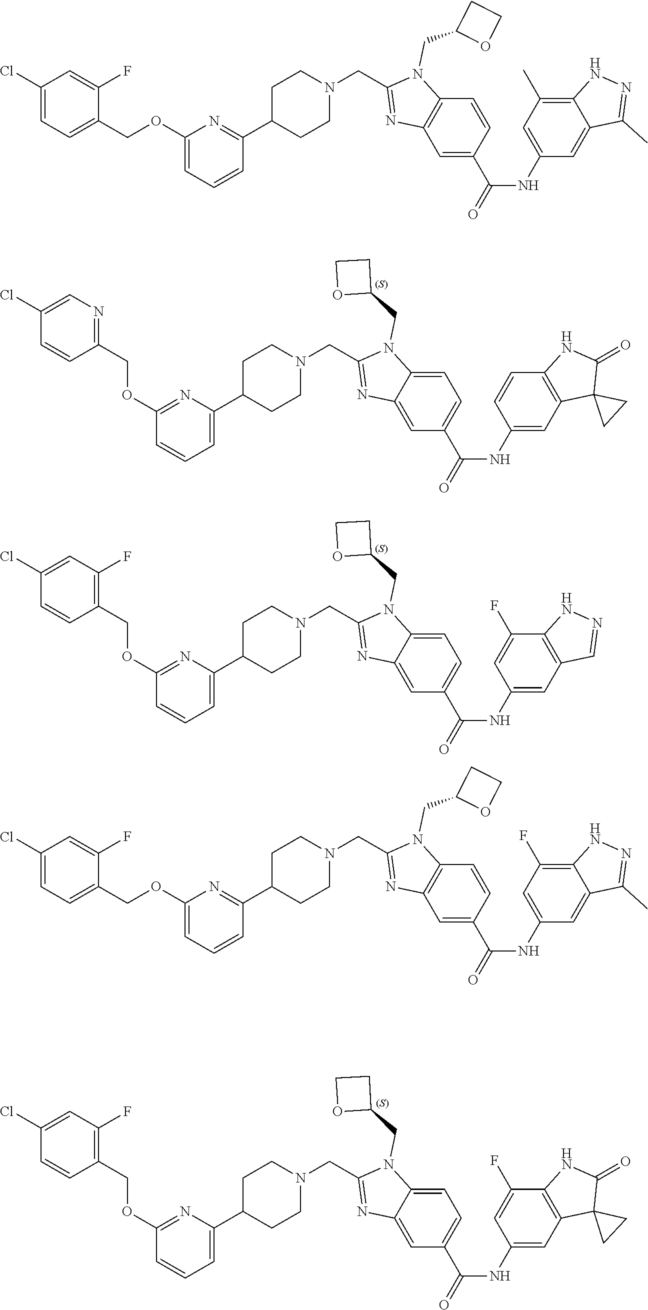

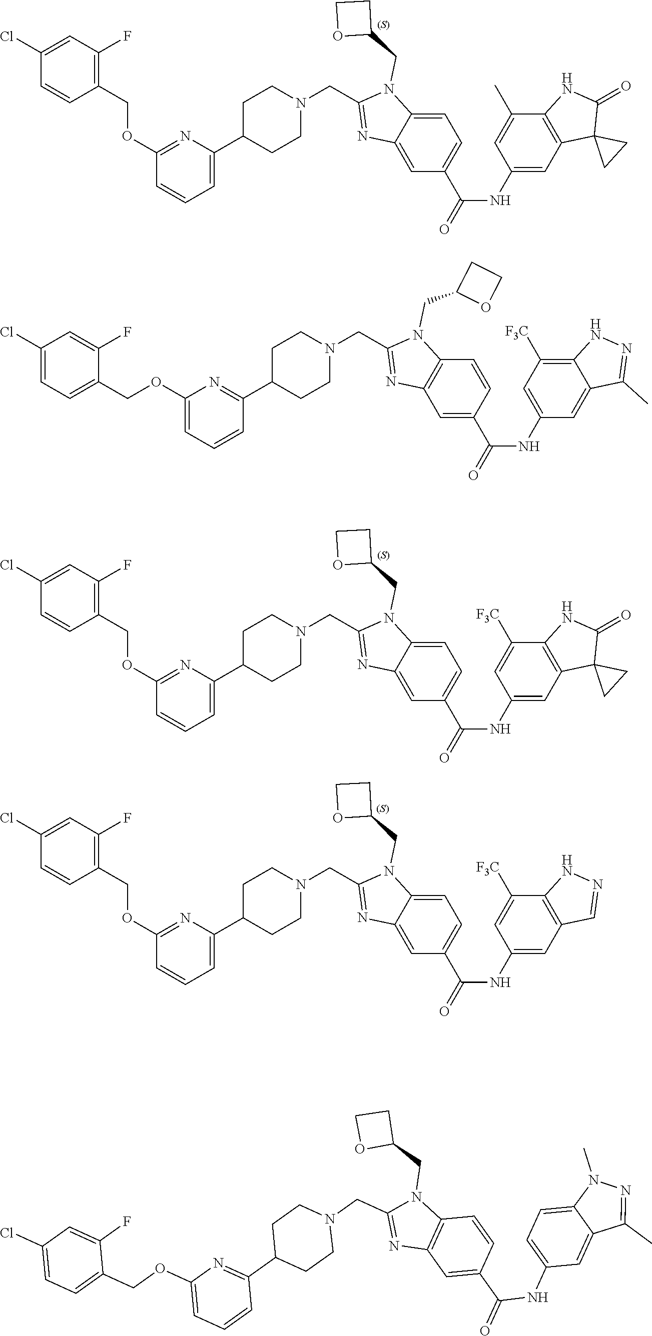

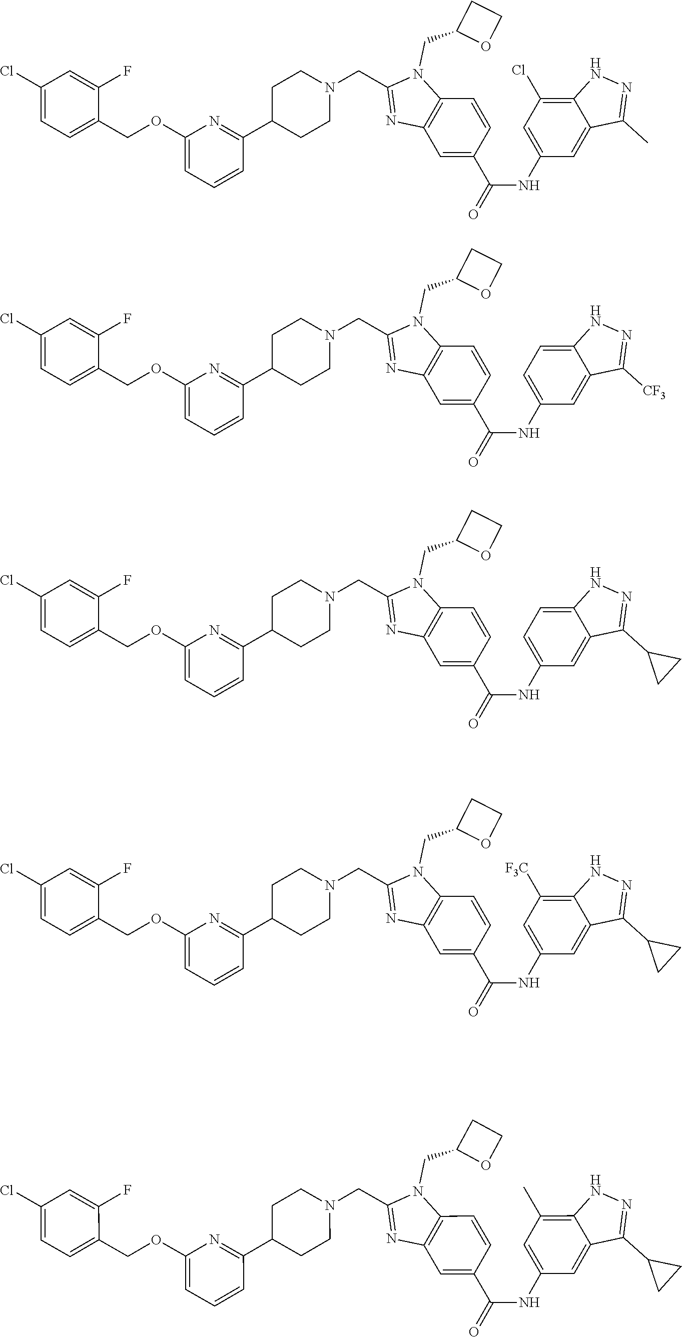

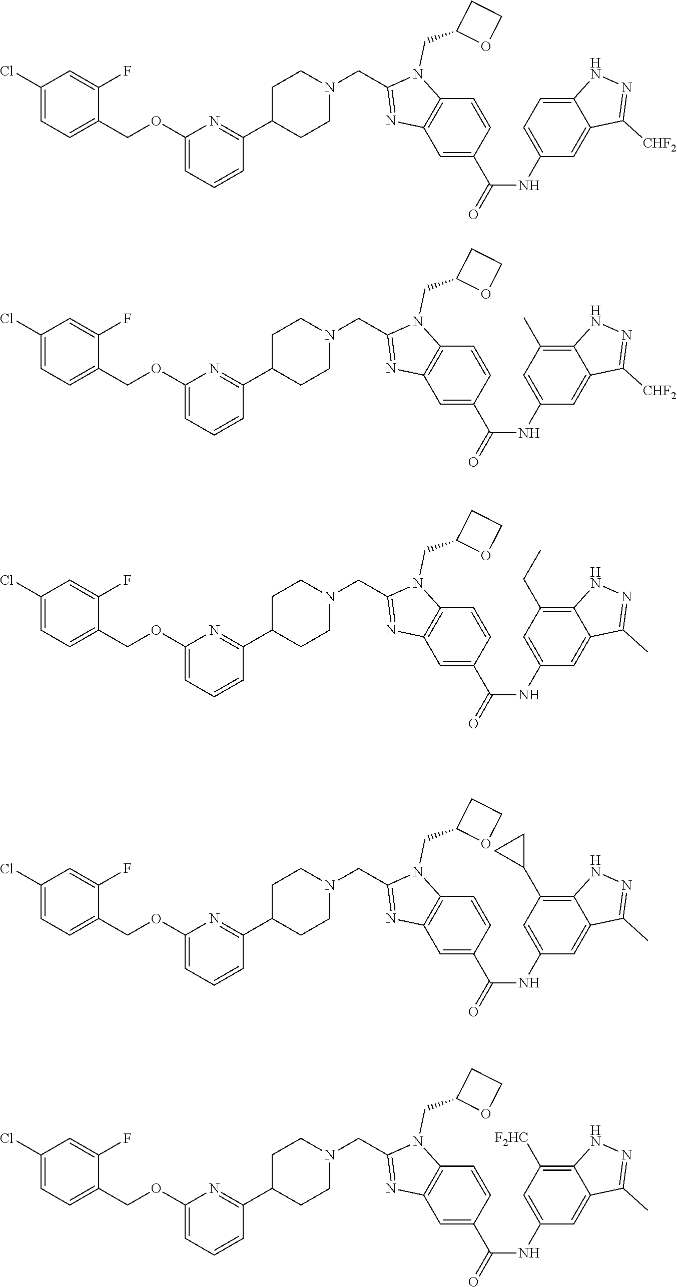

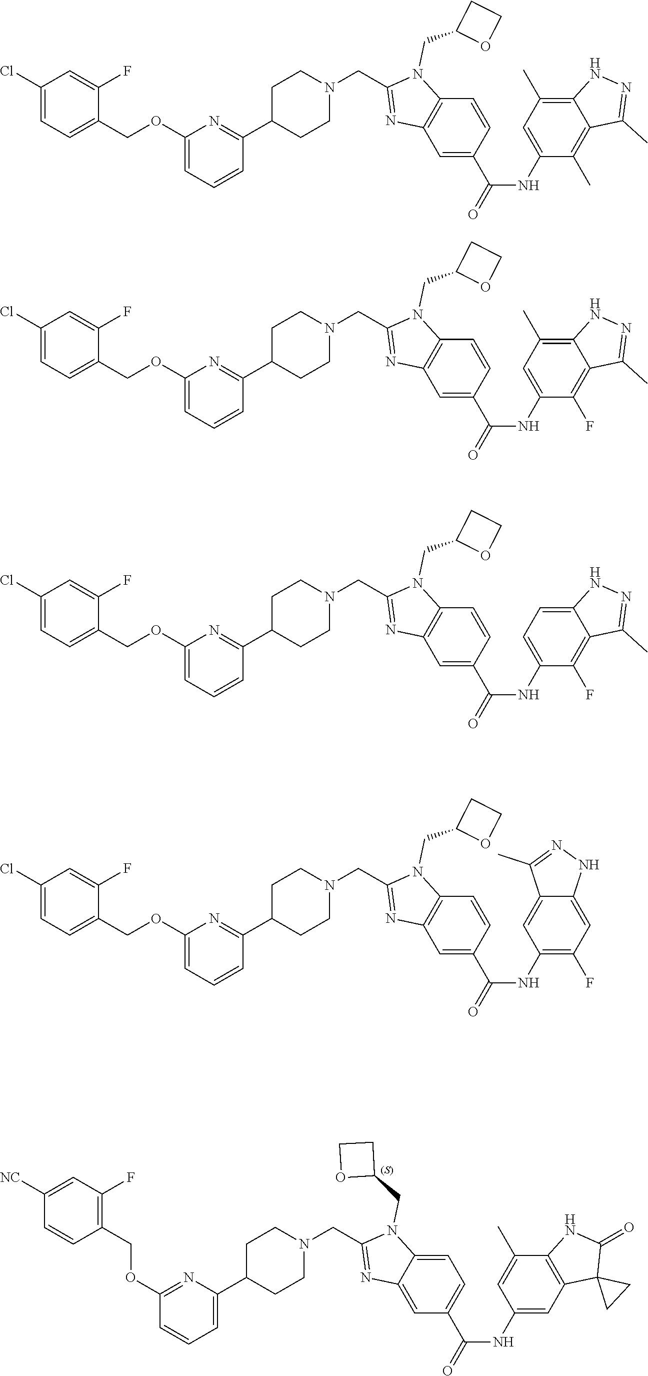

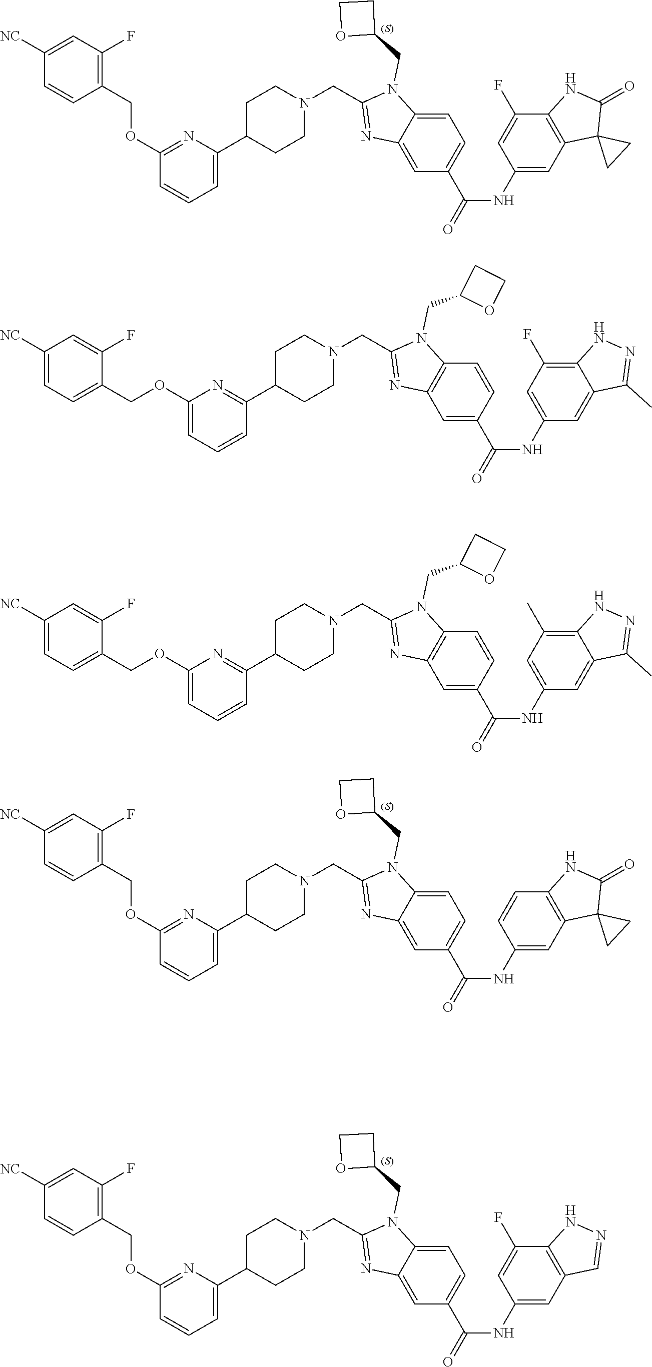

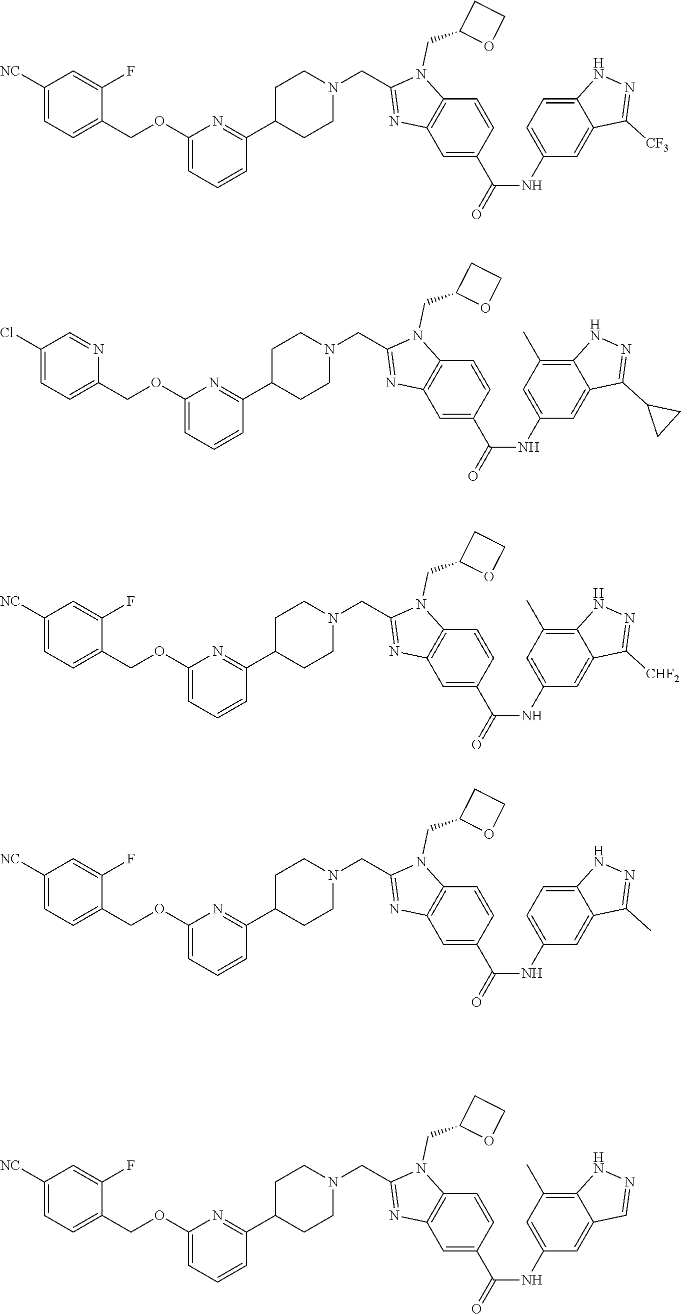

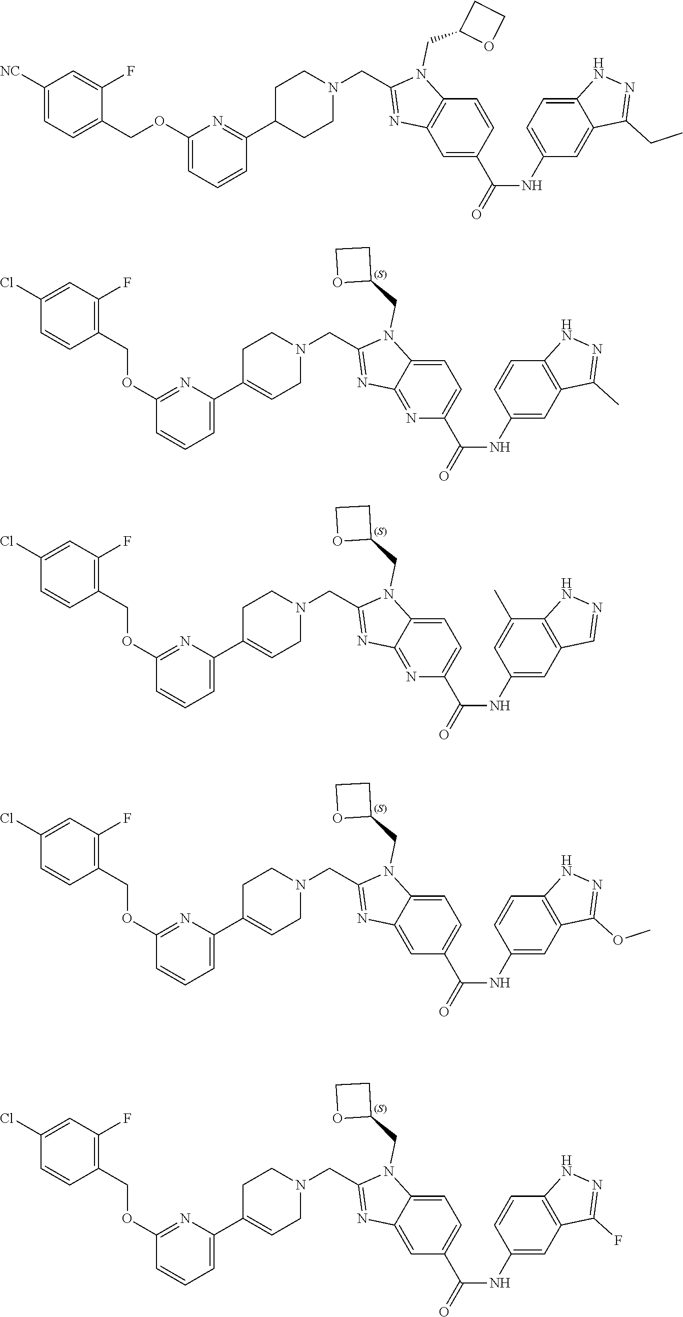

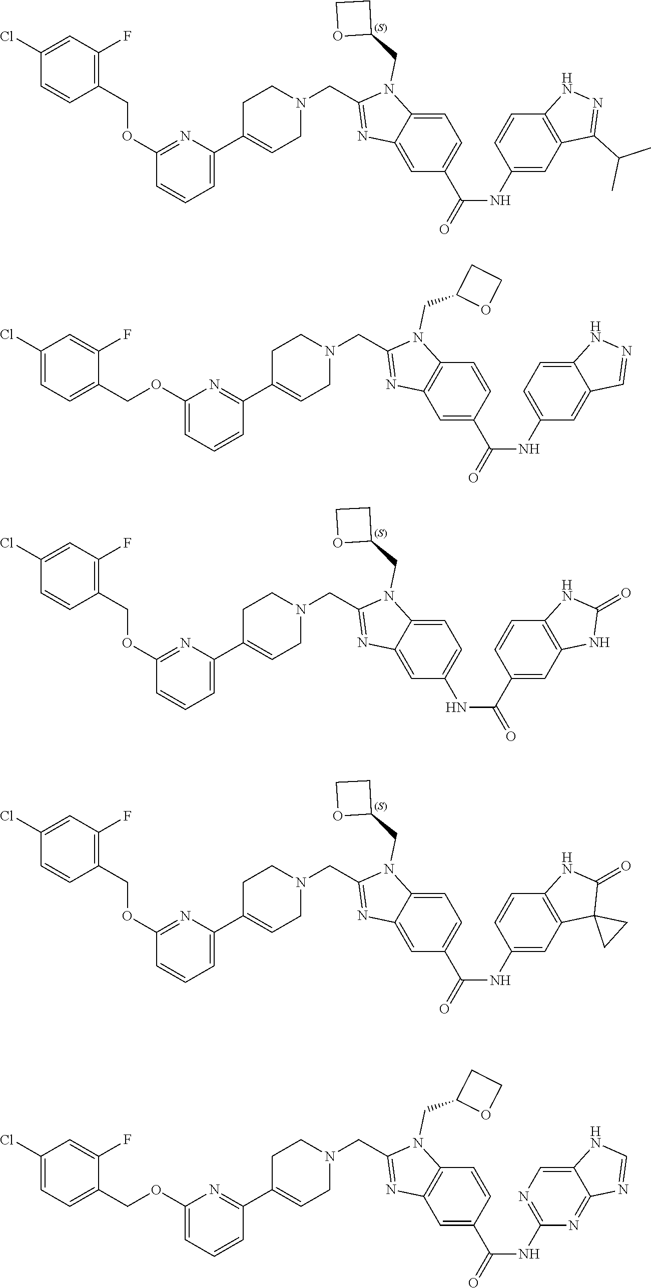

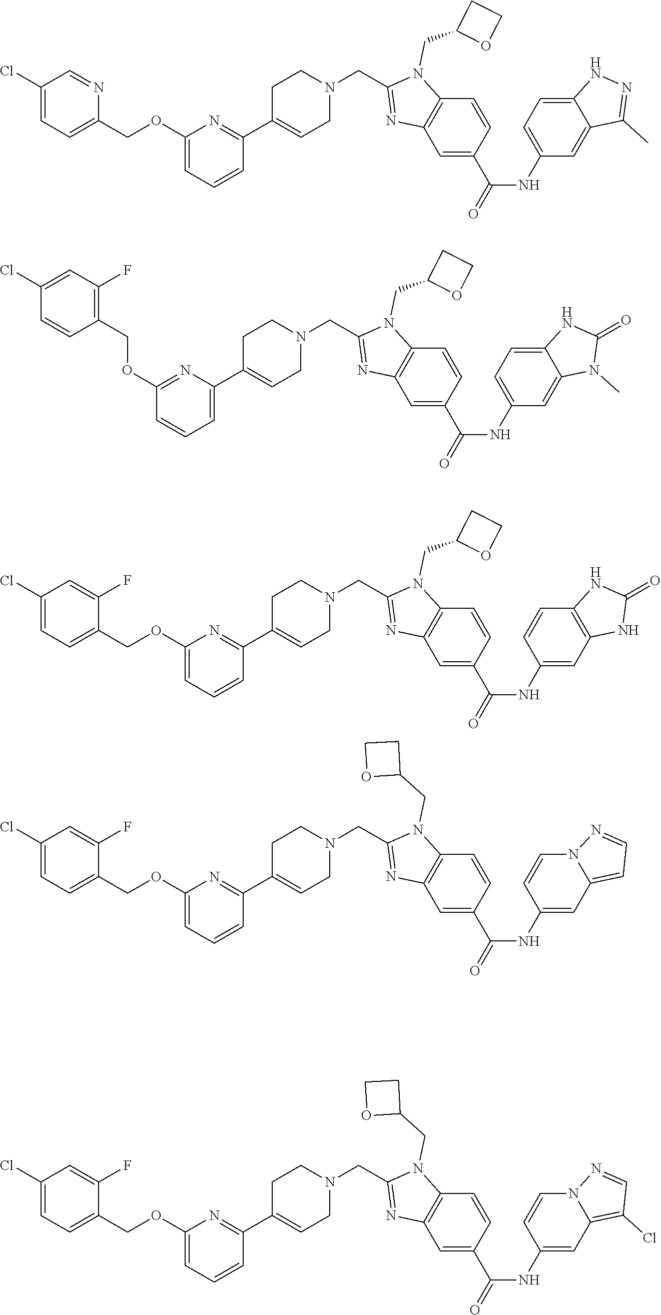 [SMILES: Cc1n[nH]c2ccc(NC(=O)c3ccc4c(c3)nc(CN3CC=C(c5cccc(OCc6ccc(Cl)cn6)n5)CC3)n4C[C@@H]3CCO3)cc12 ; Cn1c(=O)[nH]c2ccc(NC(=O)c3ccc4c(c3)nc(CN3CC=C(c5cccc(OCc6ccc(Cl)cc6F)n5)CC3)n4C[C@@H]3CCO3)cc21 ; O=C(Nc1ccc2[nH]c(=O)[nH]c2c1)c1ccc2c(c1)nc(CN1CC=C(c3cccc(OCc4ccc(Cl)cc4F)n3)CC1)n2C[C@@H]1CCO1 ; O=C(Nc1ccn2nccc2c1)c1ccc2c(c1)nc(CN1CC=C(c3cccc(OCc4ccc(Cl)cc4F)n3)CC1)n2CC1CCO1 ; O=C(Nc1ccn2ncc(Cl)c2c1)c1ccc2c(c1)nc(CN1CC=C(c3cccc(OCc4ccc(Cl)cc4F)n3)CC1)n2CC1CCO1]

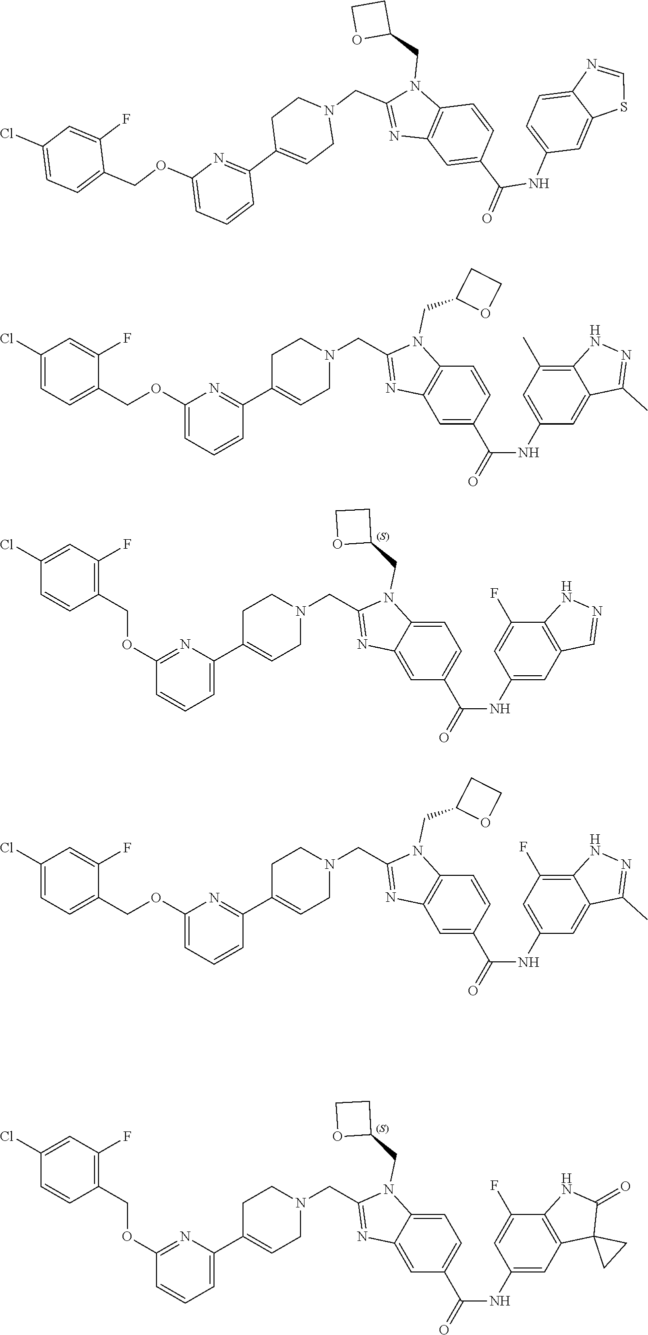

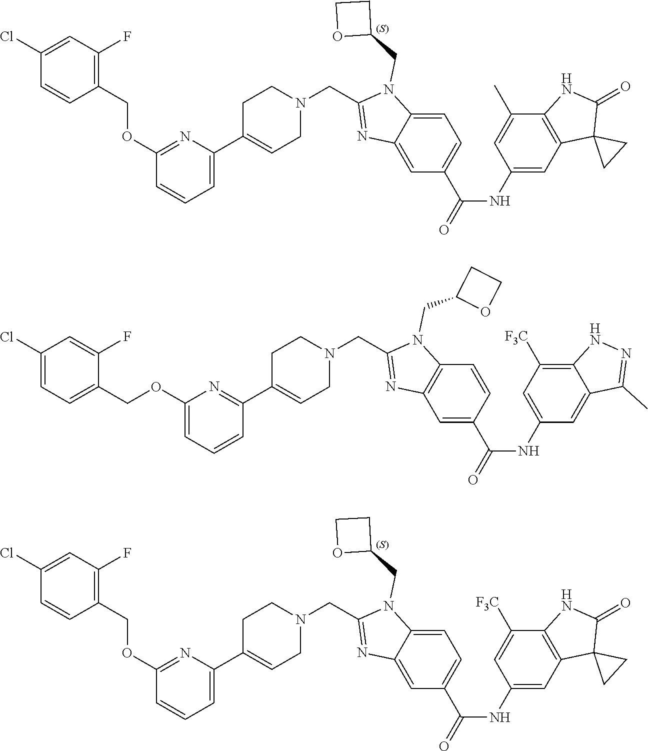

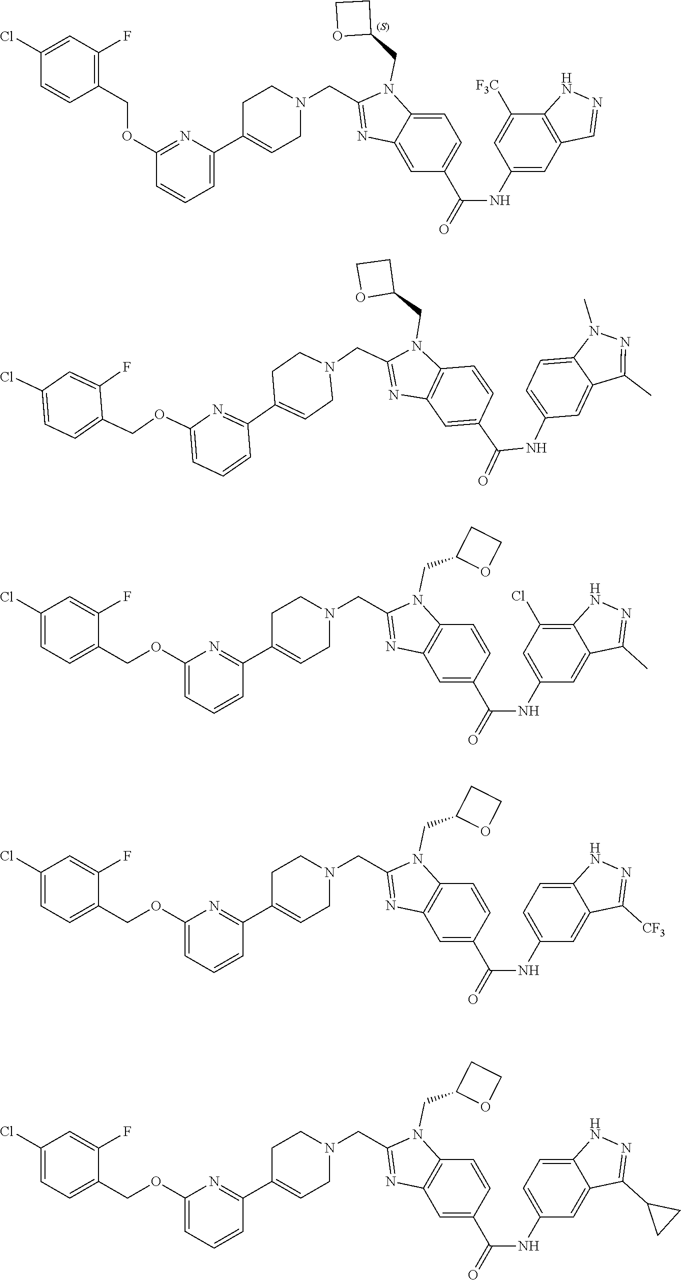

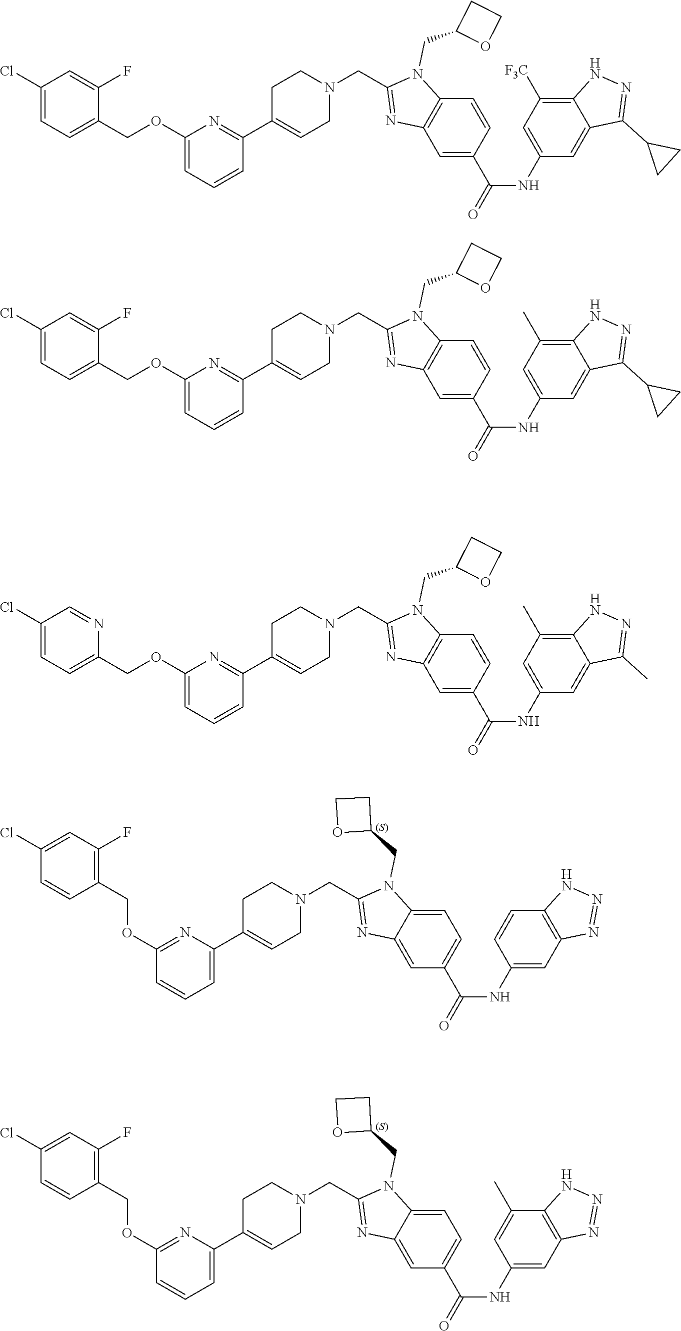

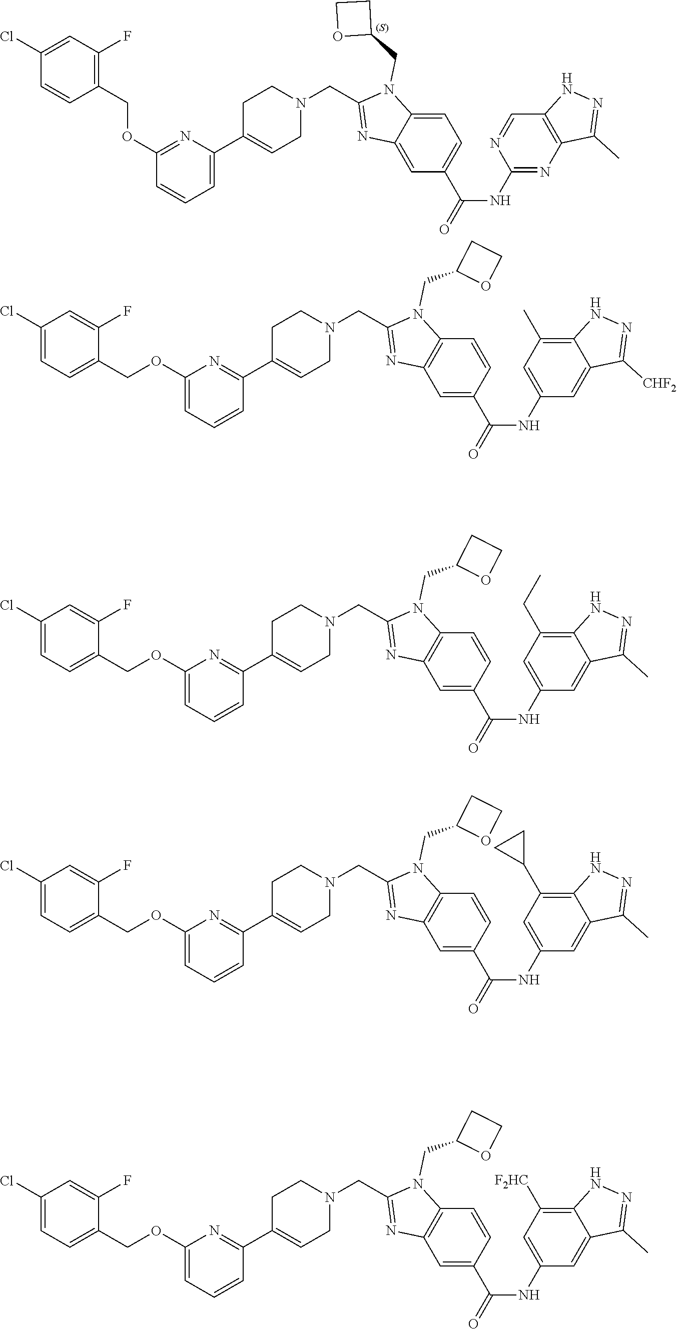

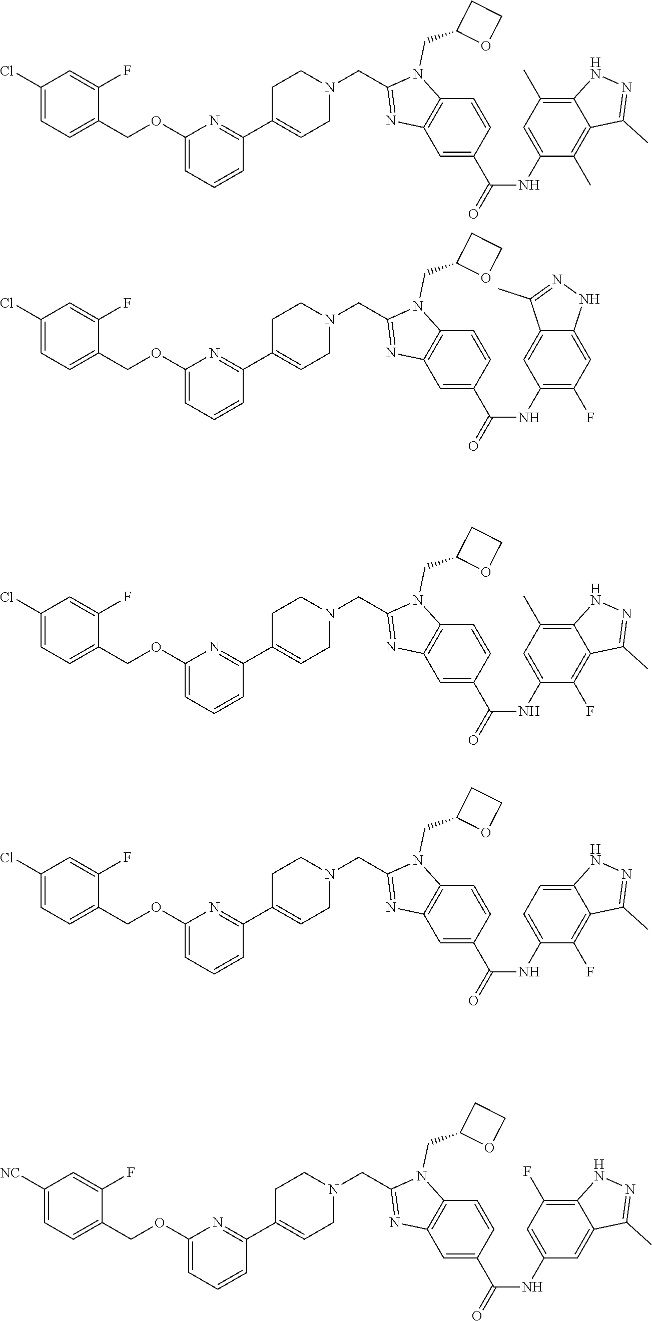

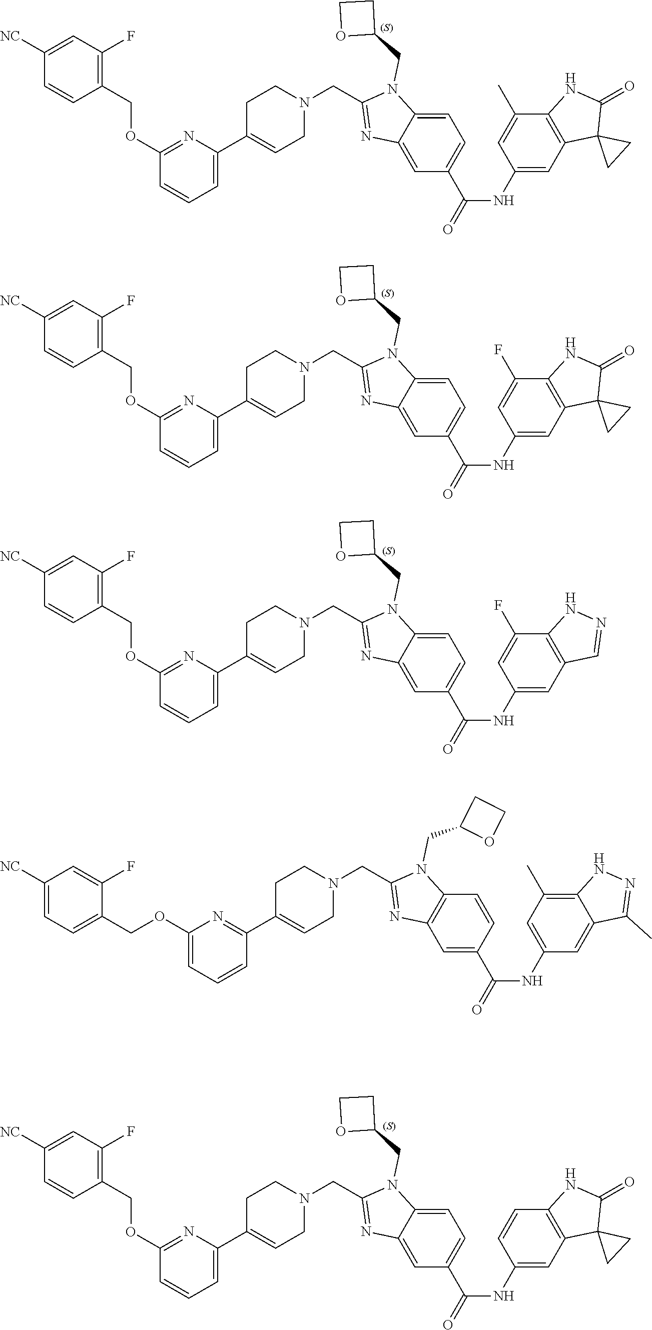

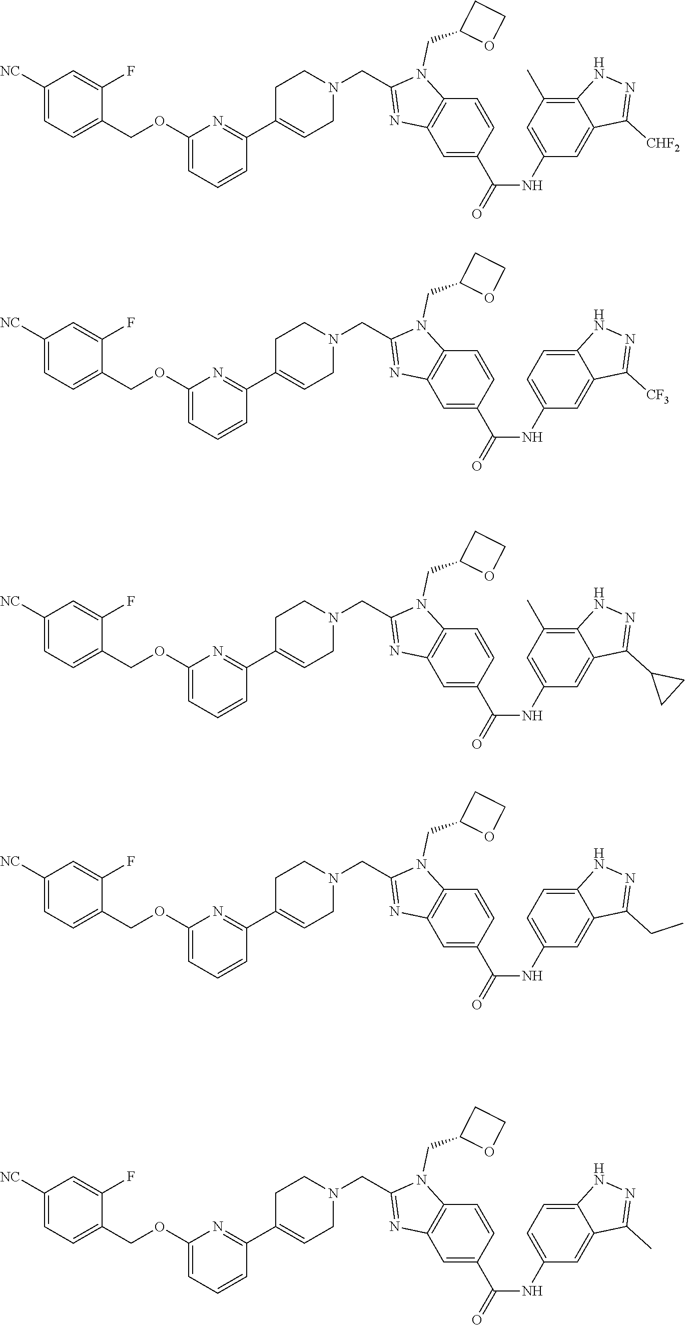

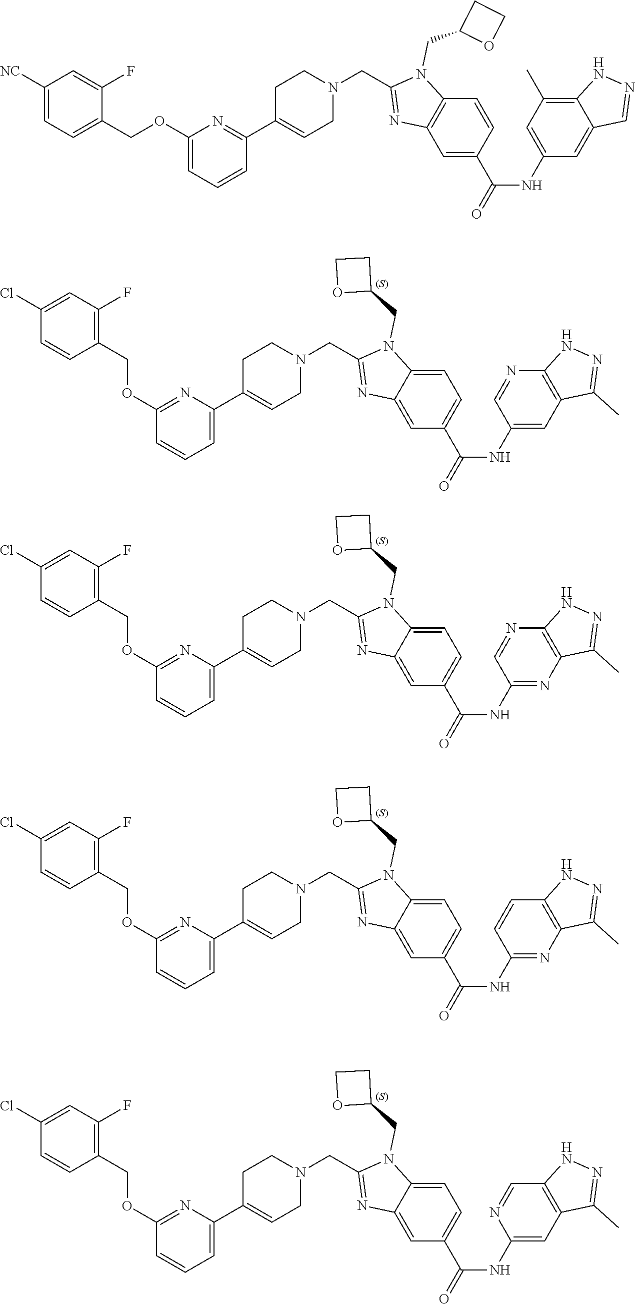

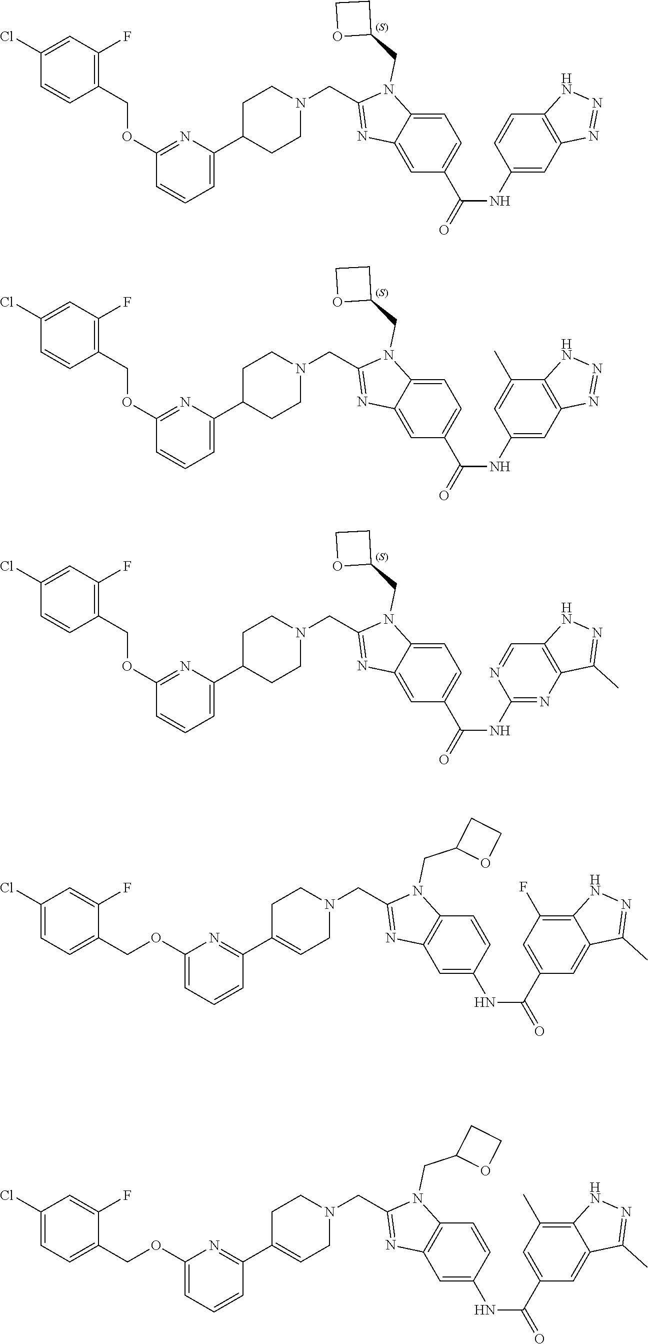

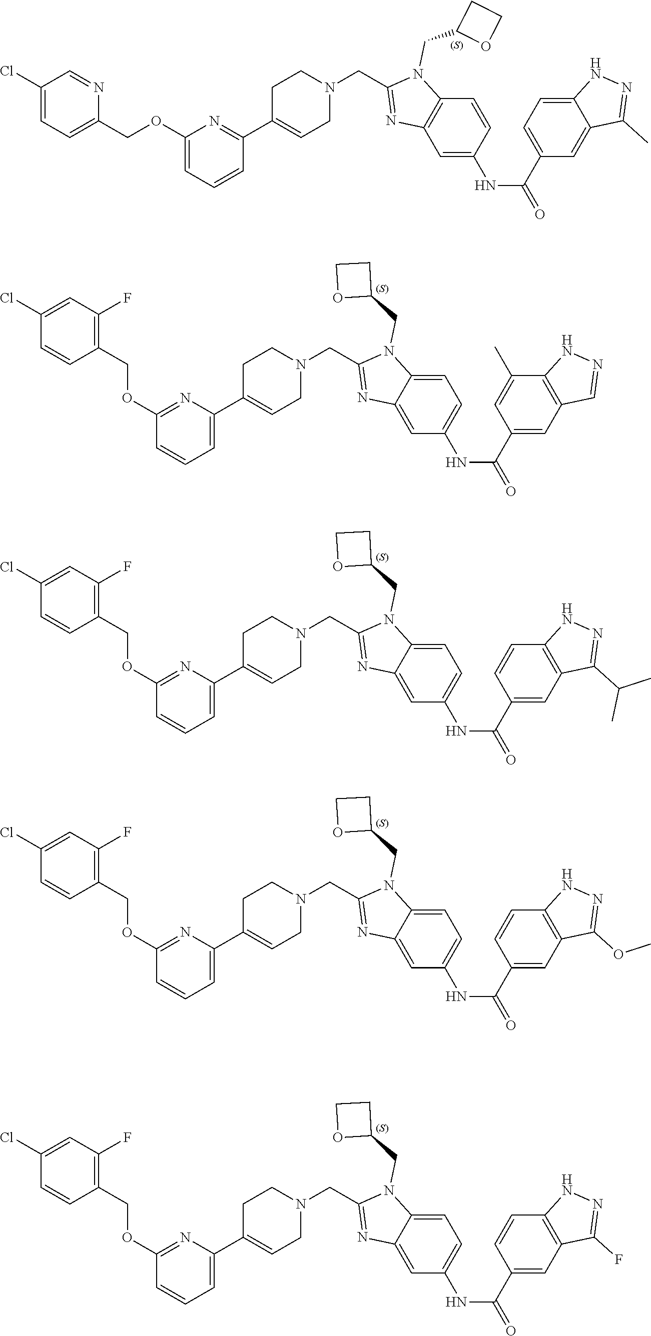

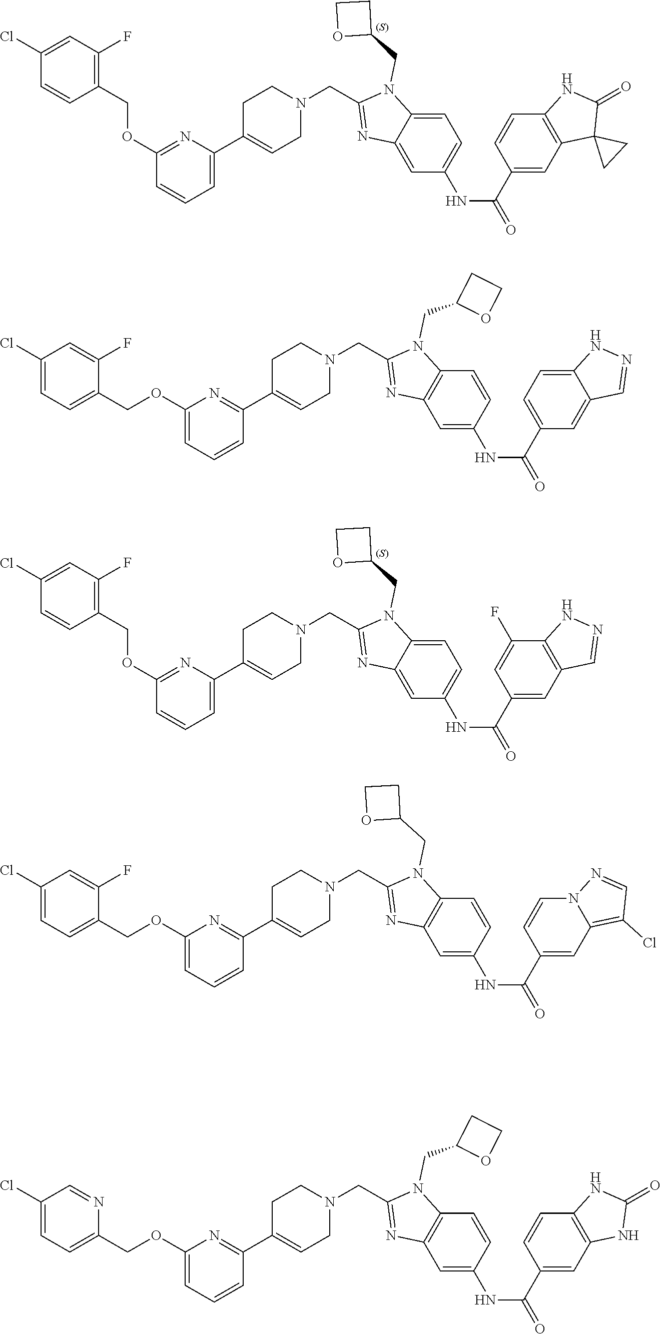

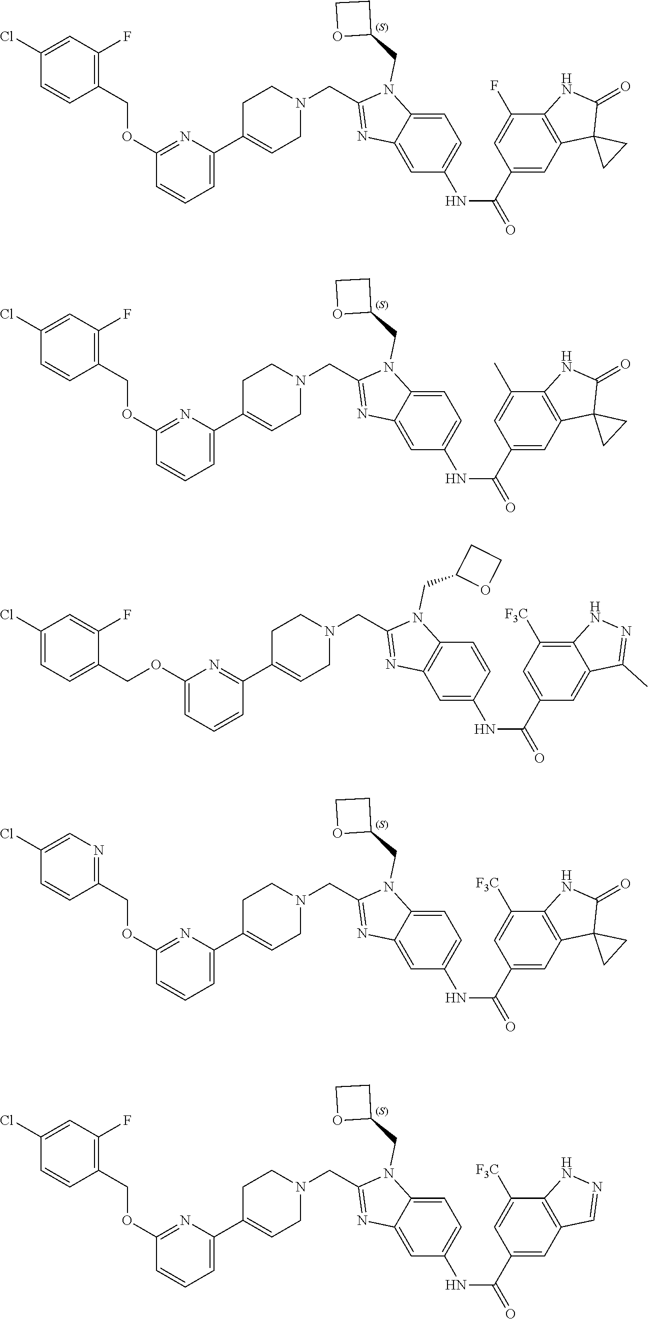

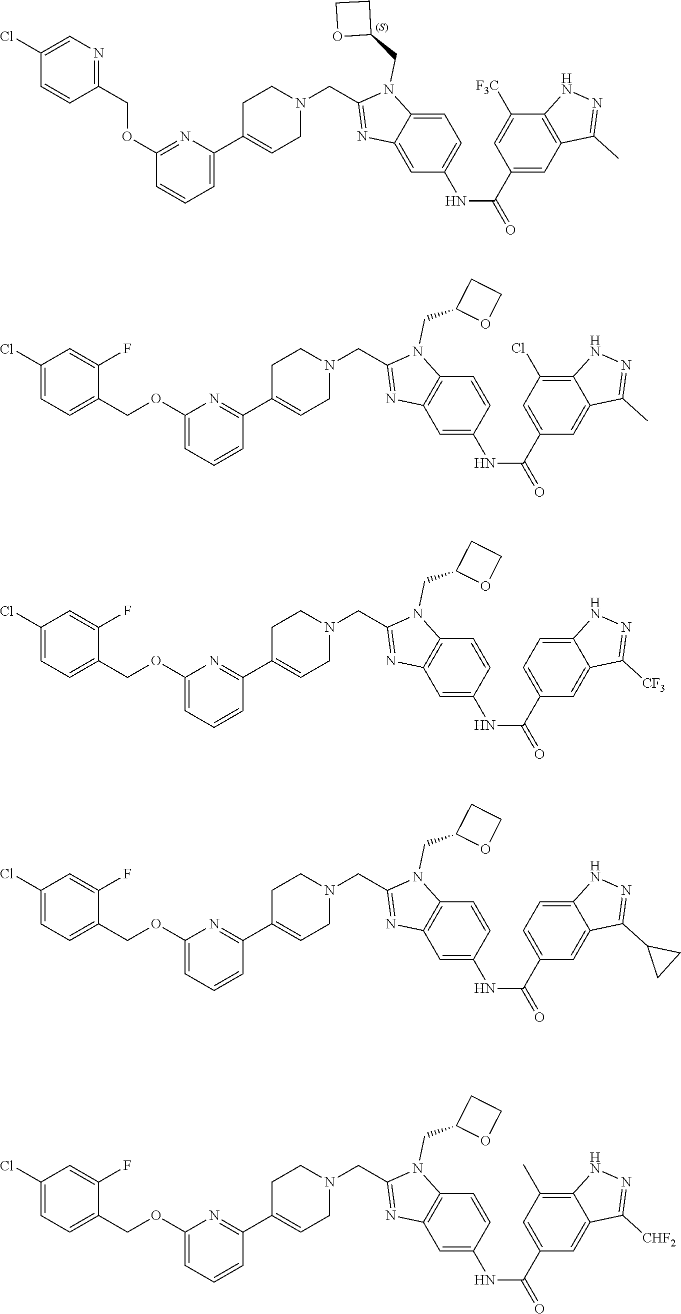

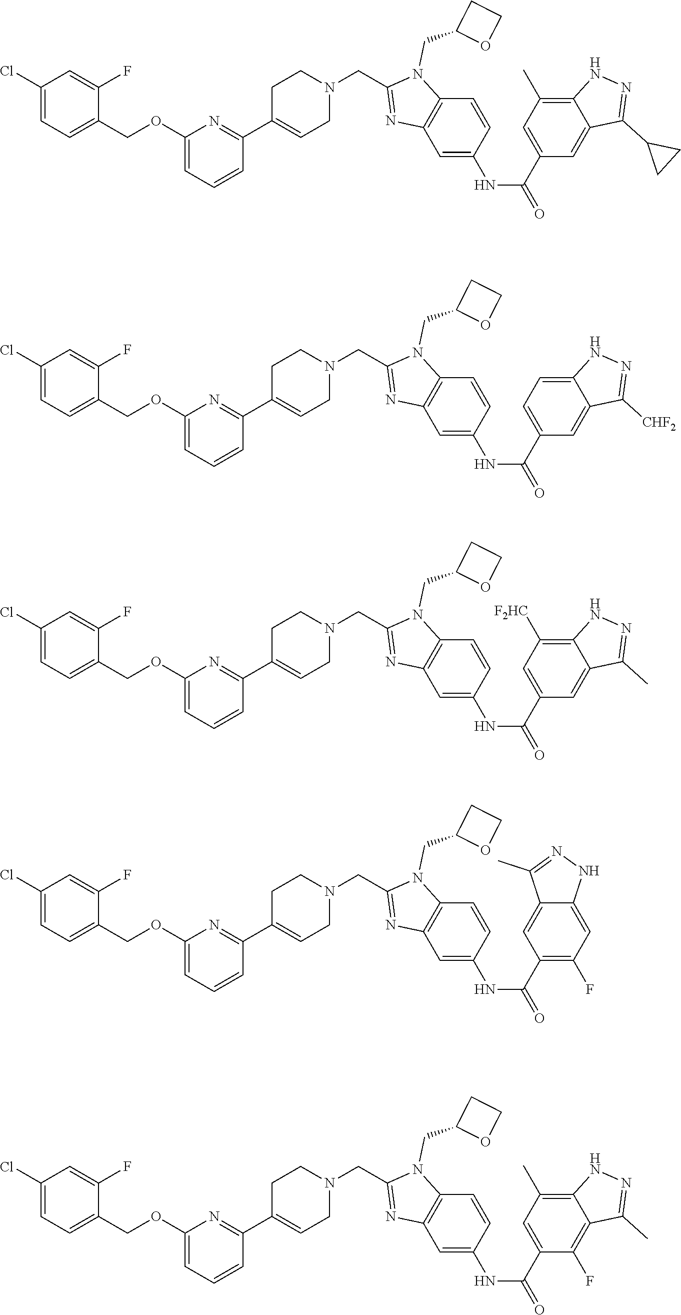 [SMILES: Cc1cc(C(=O)Nc2ccc3c(c2)nc(CN2CC=C(c4cccc(OCc5ccc(Cl)cc5F)n4)CC2)n3C[C@@H]2CCO2)cc2c(C3CC3)n[nH]c12 ; O=C(Nc1ccc2c(c1)nc(CN1CC=C(c3cccc(OCc4ccc(Cl)cc4F)n3)CC1)n2C[C@@H]1CCO1)c1ccc2[nH]nc(C(F)F)c2c1 ; Cc1n[nH]c2c(C(F)F)cc(C(=O)Nc3ccc4c(c3)nc(CN3CC=C(c5cccc(OCc6ccc(Cl)cc6F)n5)CC3)n4C[C@@H]3CCO3)cc12 ; Cc1n[nH]c2cc(F)c(C(=O)Nc3ccc4c(c3)nc(CN3CC=C(c5cccc(OCc6ccc(Cl)cc6F)n5)CC3)n4C[C@@H]3CCO3)cc12 ; Cc1cc(C(=O)Nc2ccc3c(c2)nc(CN2CC=C(c4cccc(OCc5ccc(Cl)cc5F)n4)CC2)n3C[C@@H]2CCO2)c(F)c2c(C)n[nH]c12]

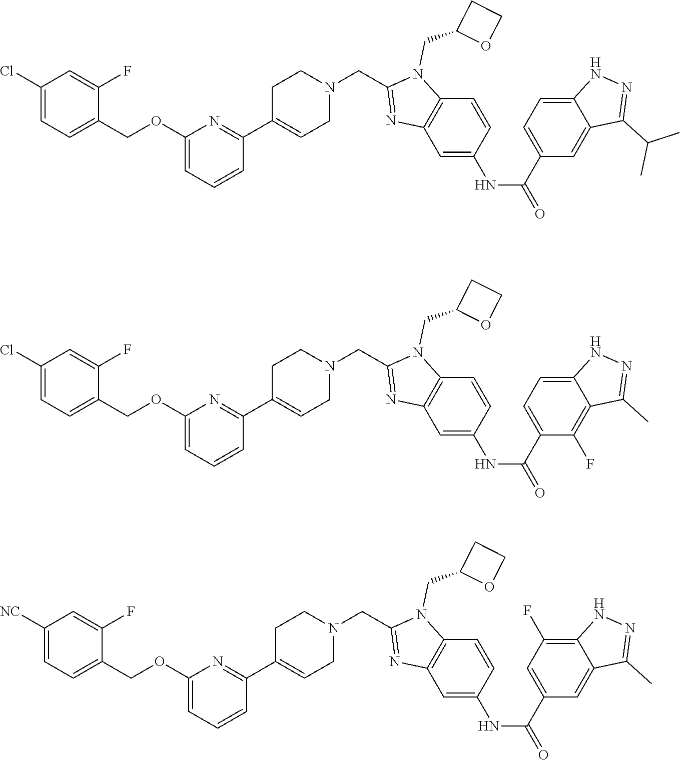 [SMILES: CC(C)c1n[nH]c2ccc(C(=O)Nc3ccc4c(c3)nc(CN3CC=C(c5cccc(OCc6ccc(Cl)cc6F)n5)CC3)n4C[C@@H]3CCO3)cc12 ; Cc1n[nH]c2ccc(C(=O)Nc3ccc4c(c3)nc(CN3CC=C(c5cccc(OCc6ccc(Cl)cc6F)n5)CC3)n4C[C@@H]3CCO3)c(F)c12 ; Cc1n[nH]c2c(F)cc(C(=O)Nc3ccc4c(c3)nc(CN3CC=C(c5cccc(OCc6ccc(C#N)cc6F)n5)CC3)n4C[C@@H]3CCO3)cc12]

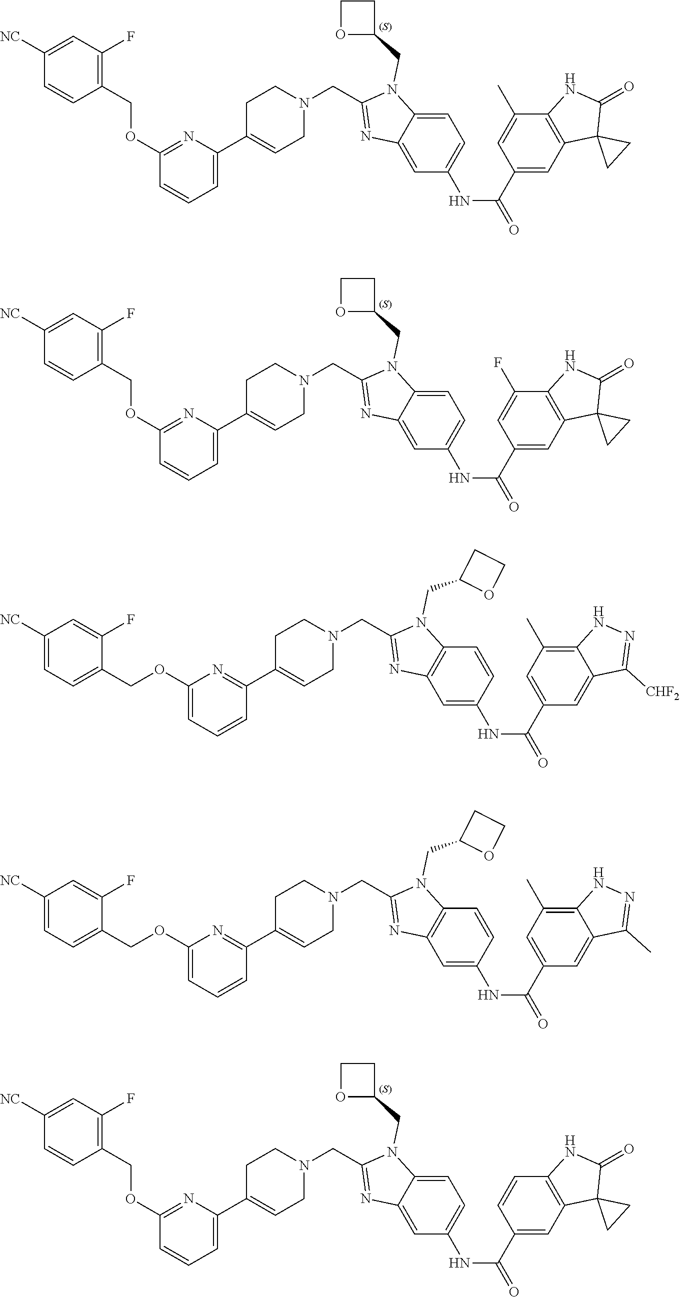

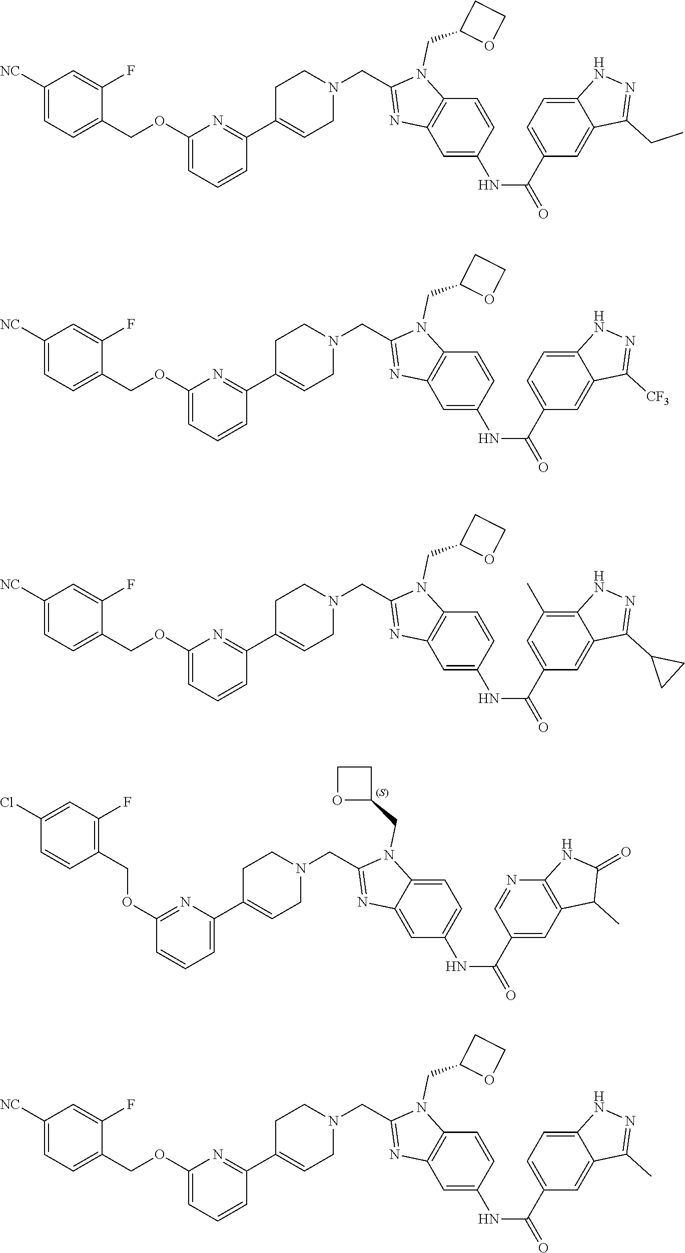

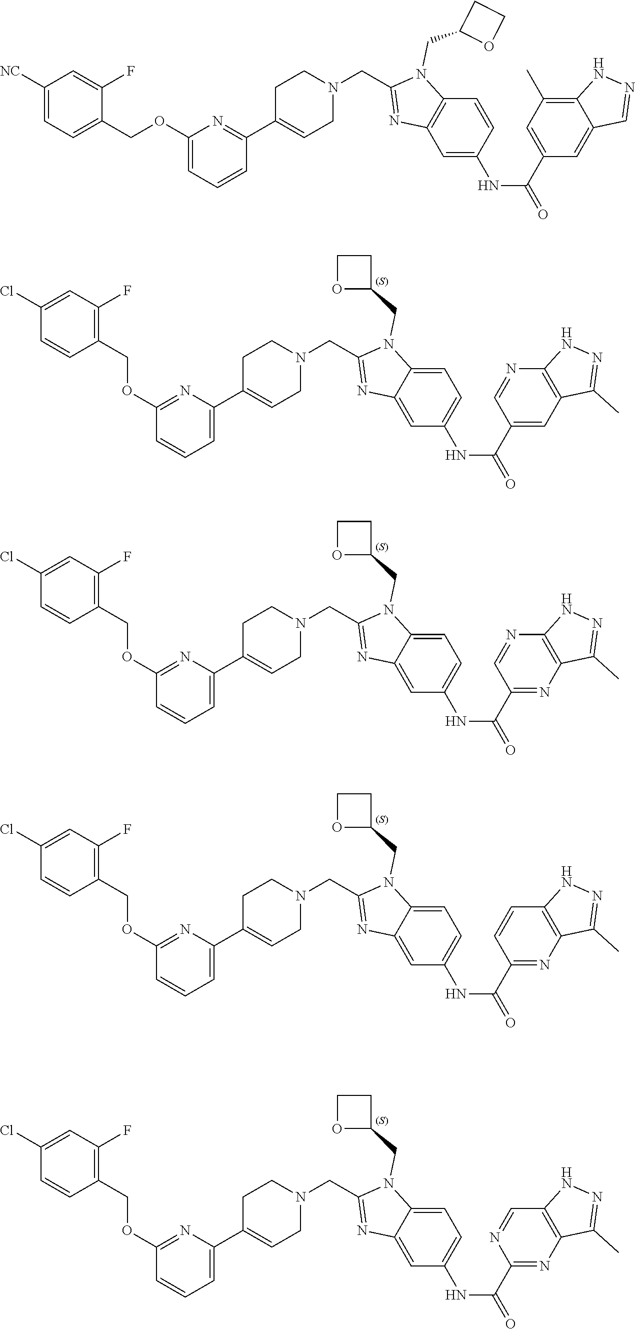

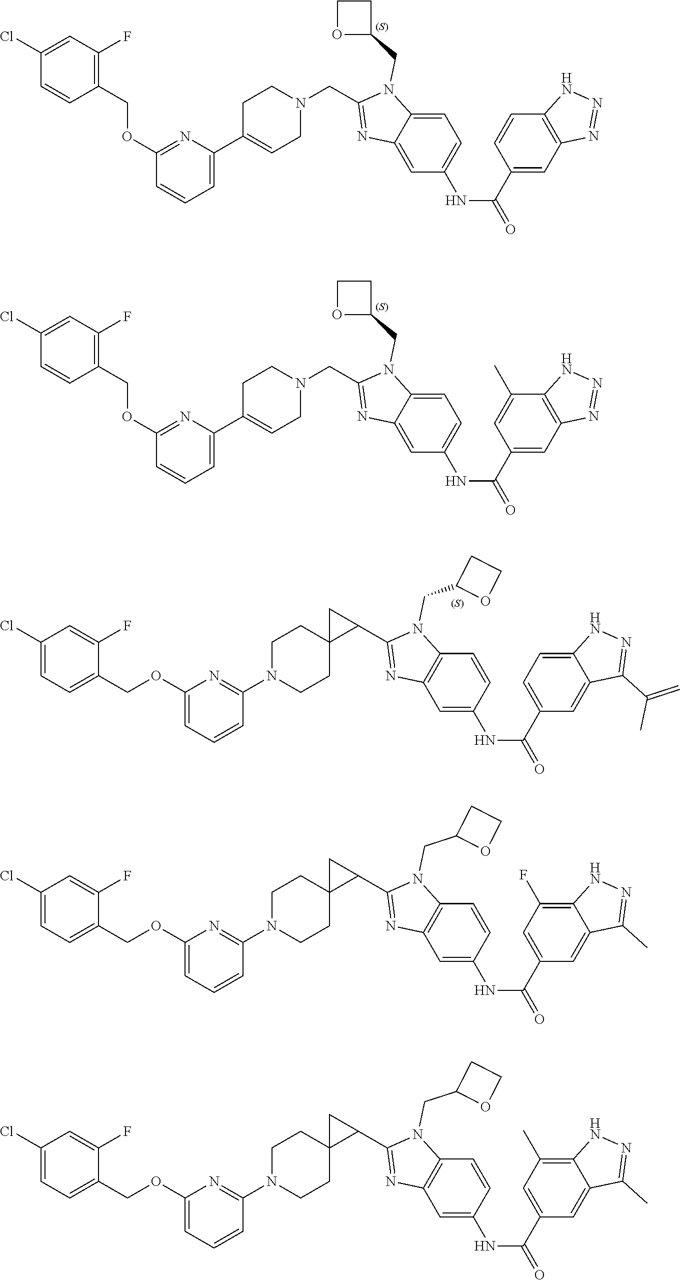

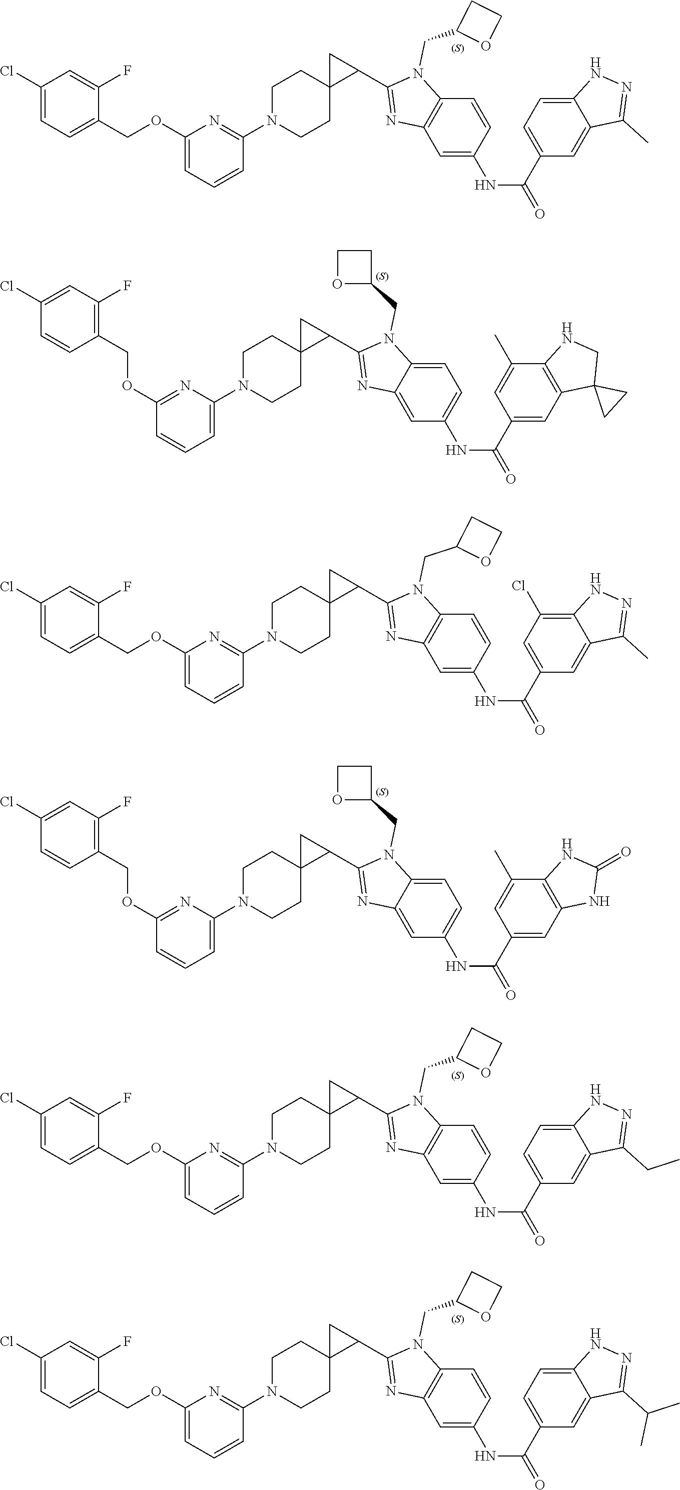

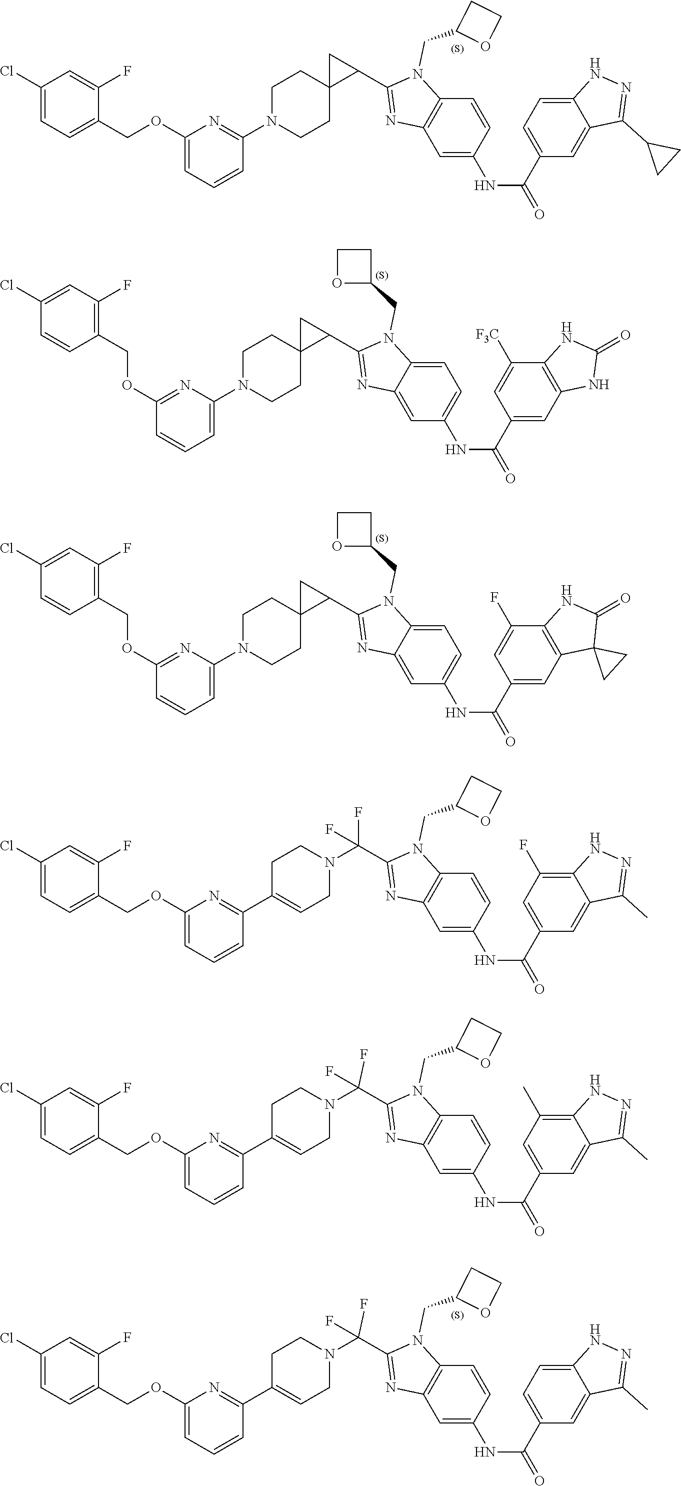

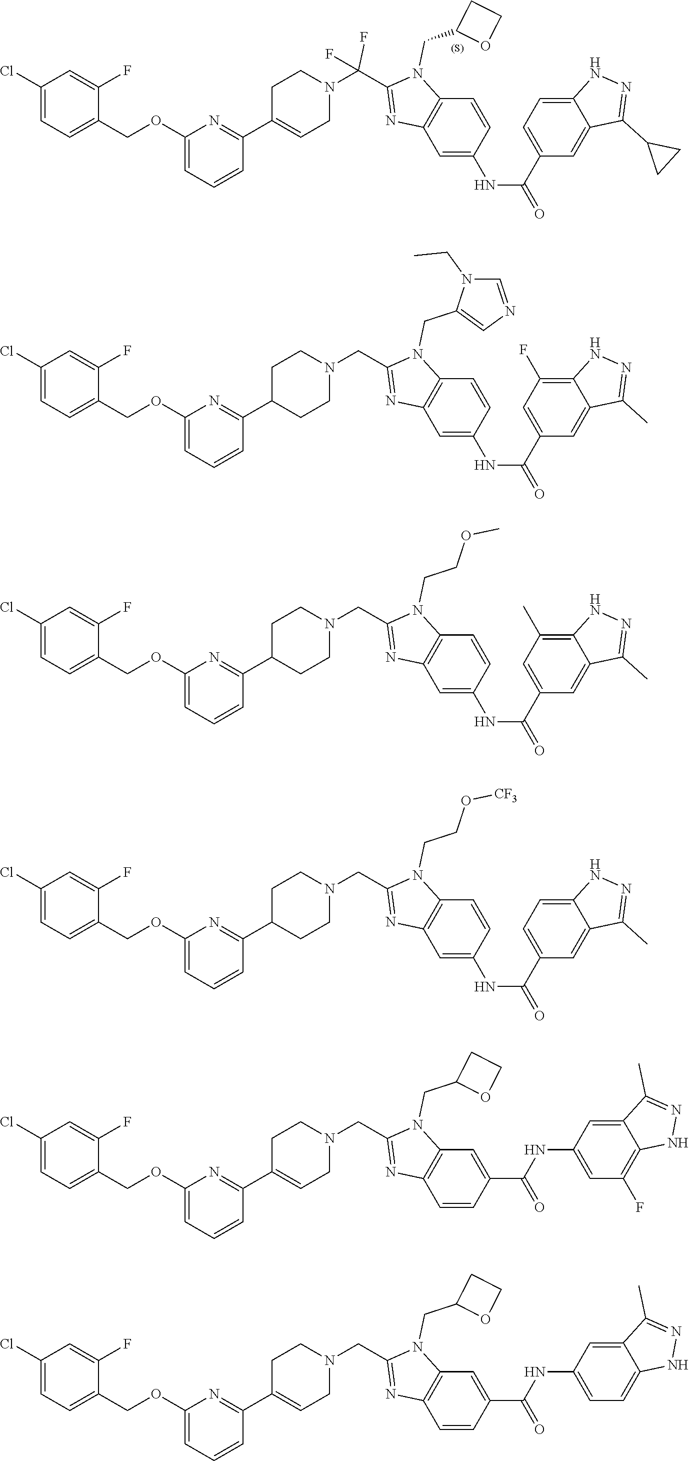

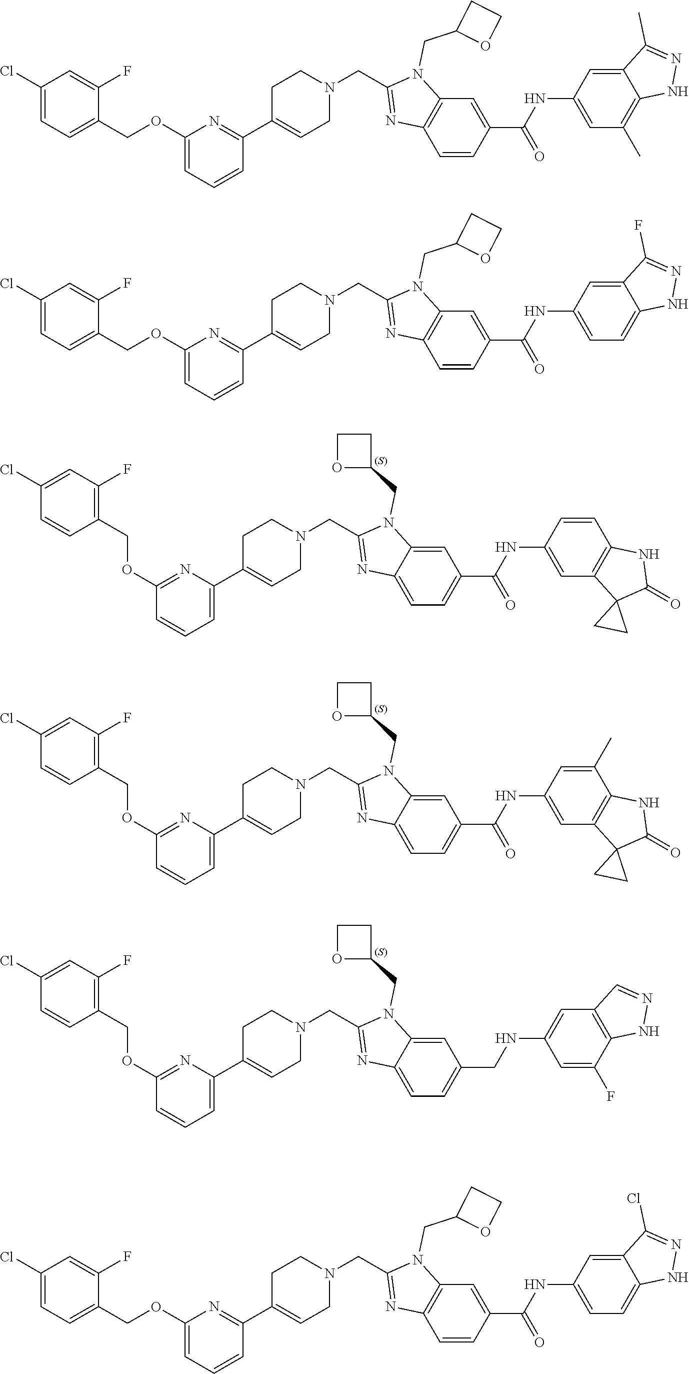

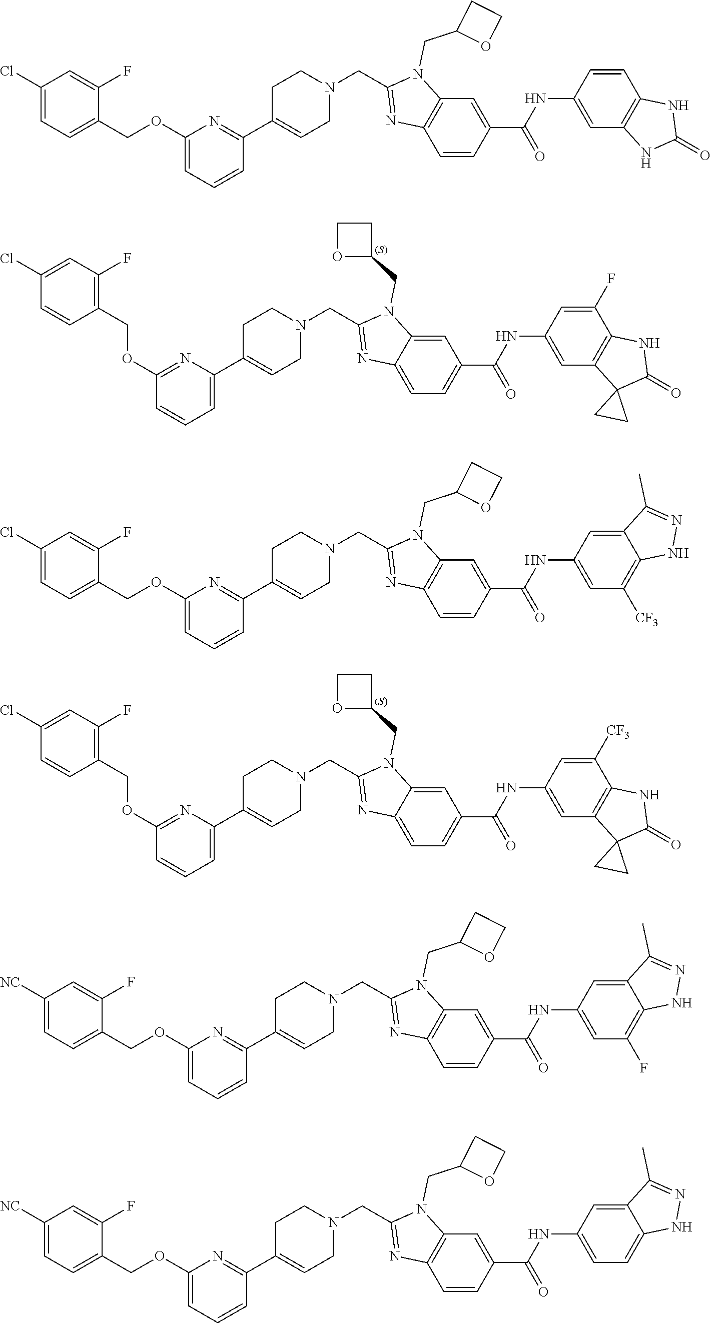

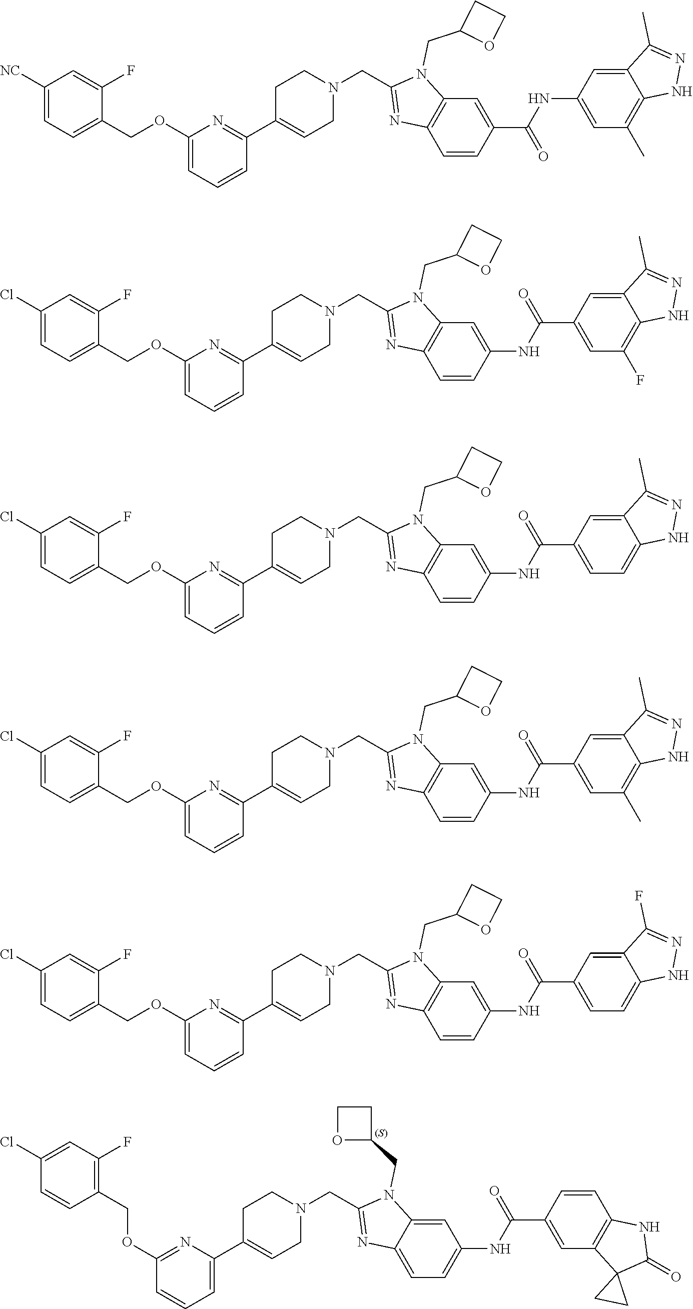

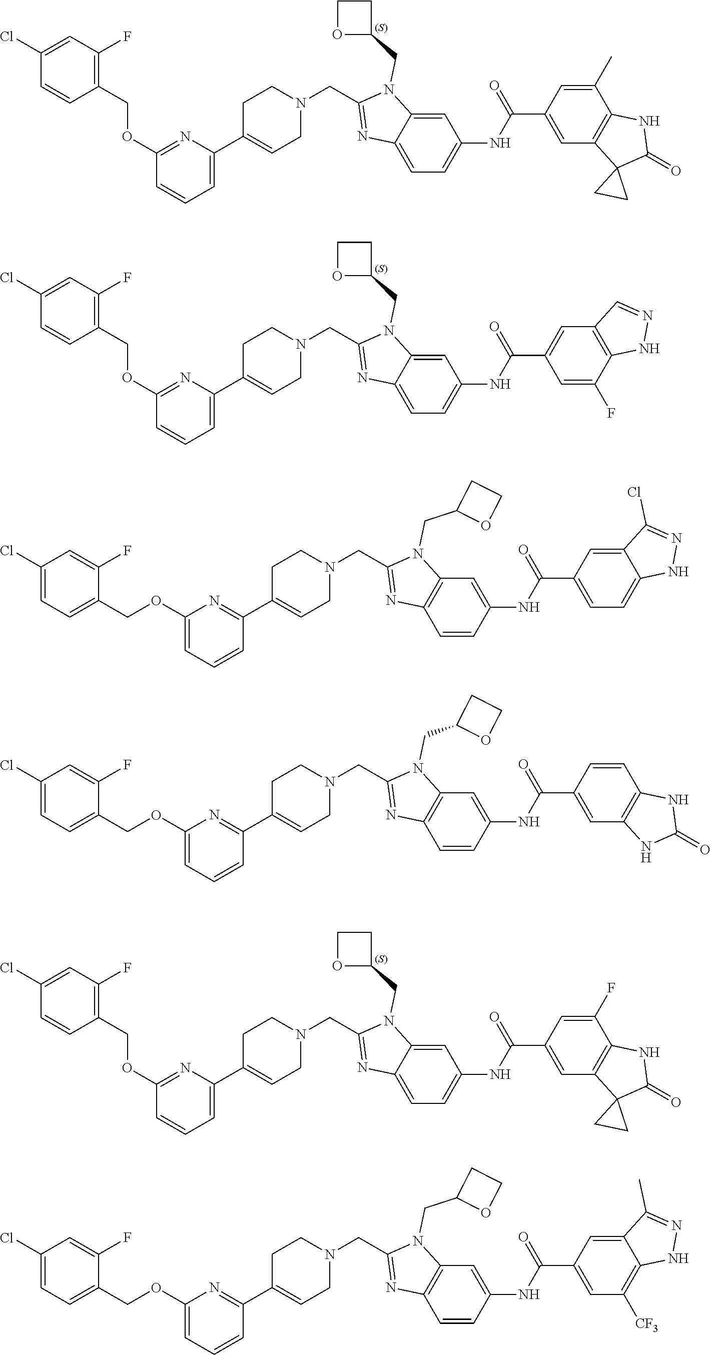

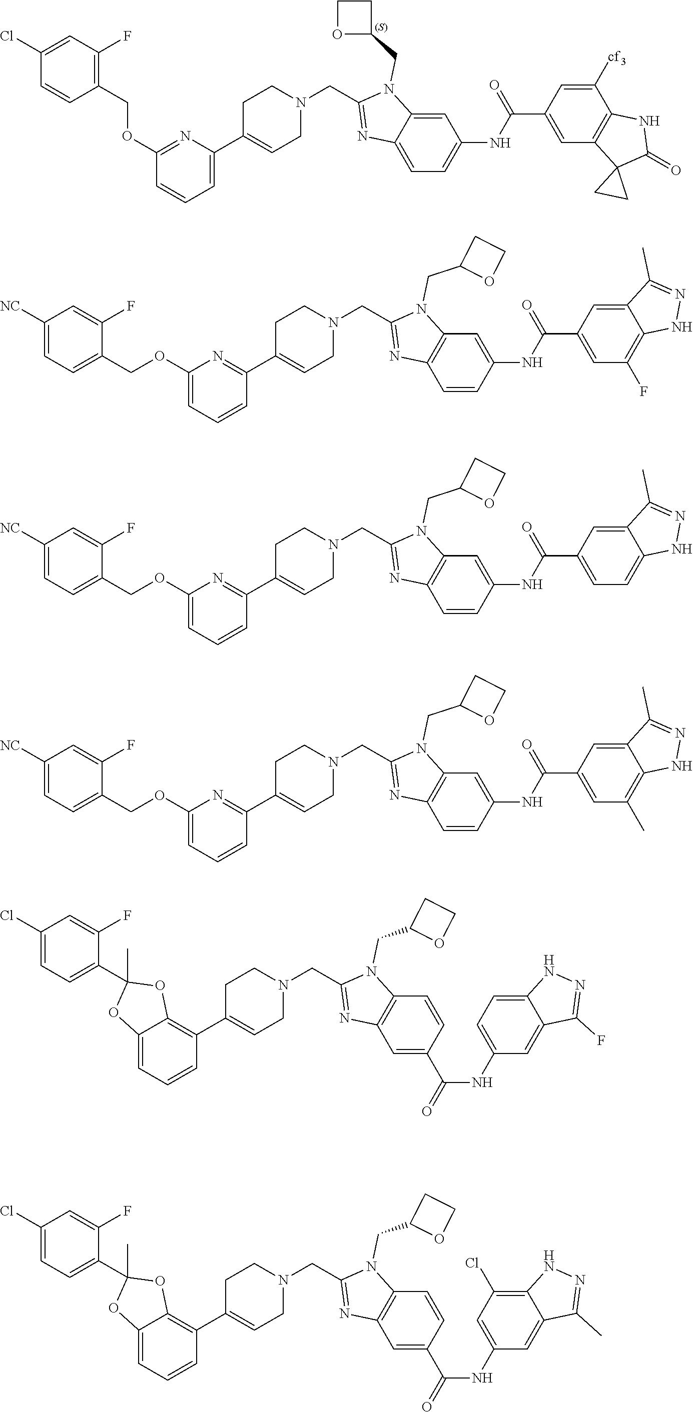

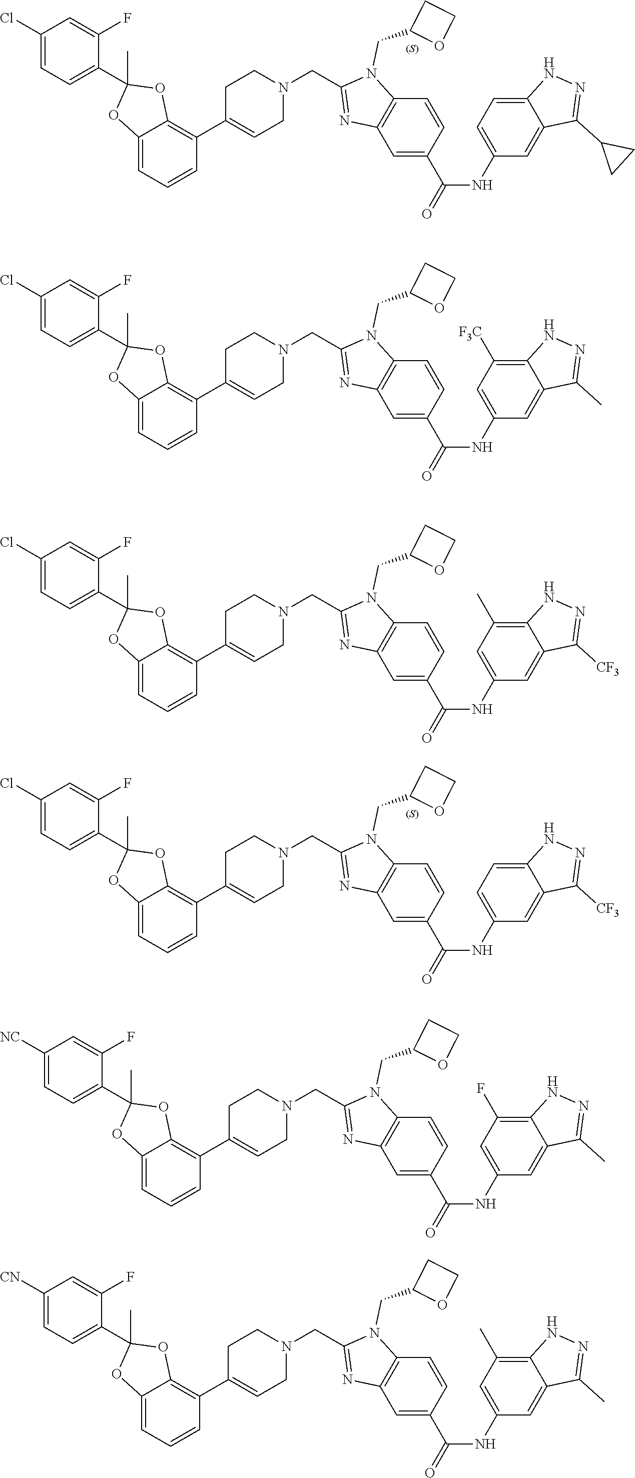

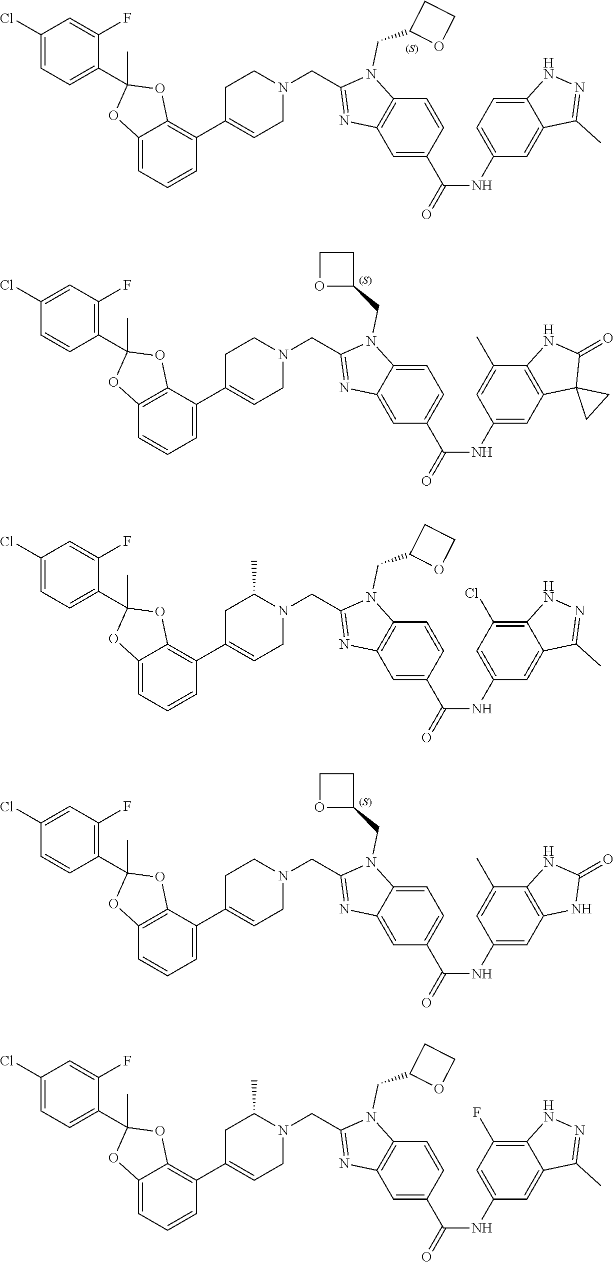

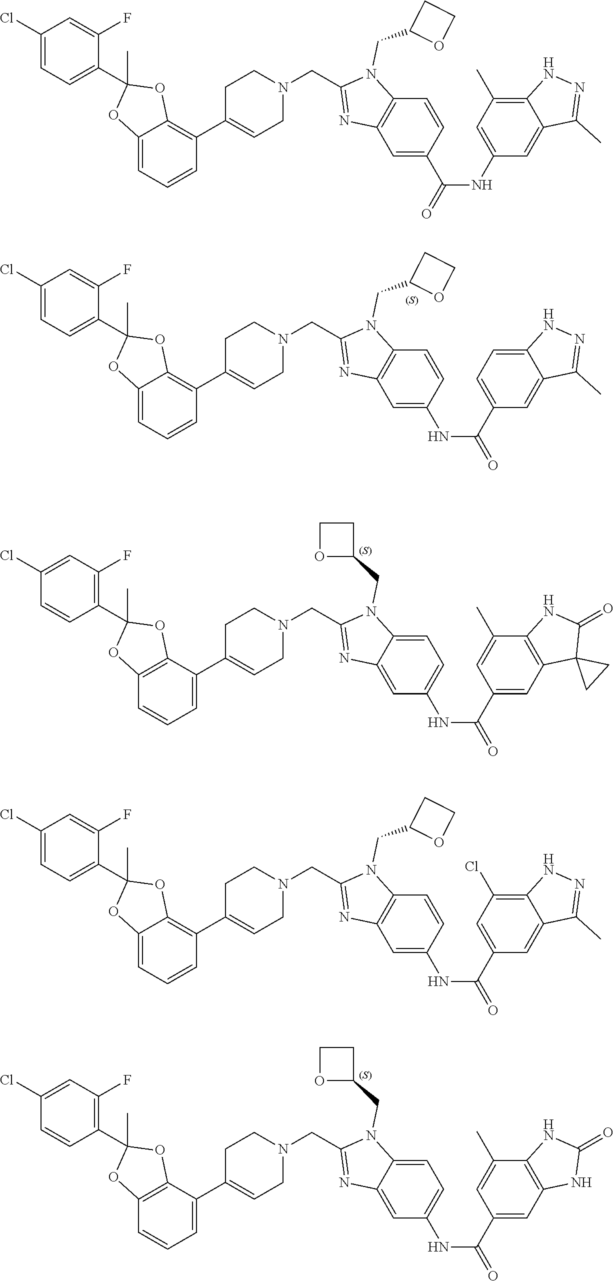

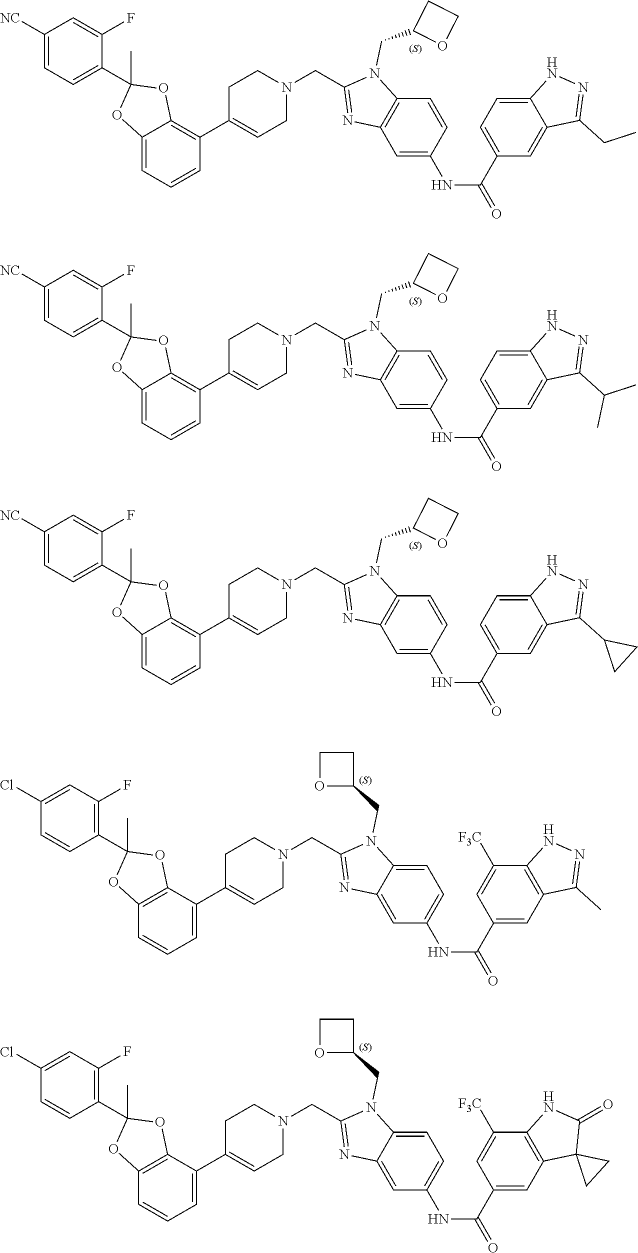

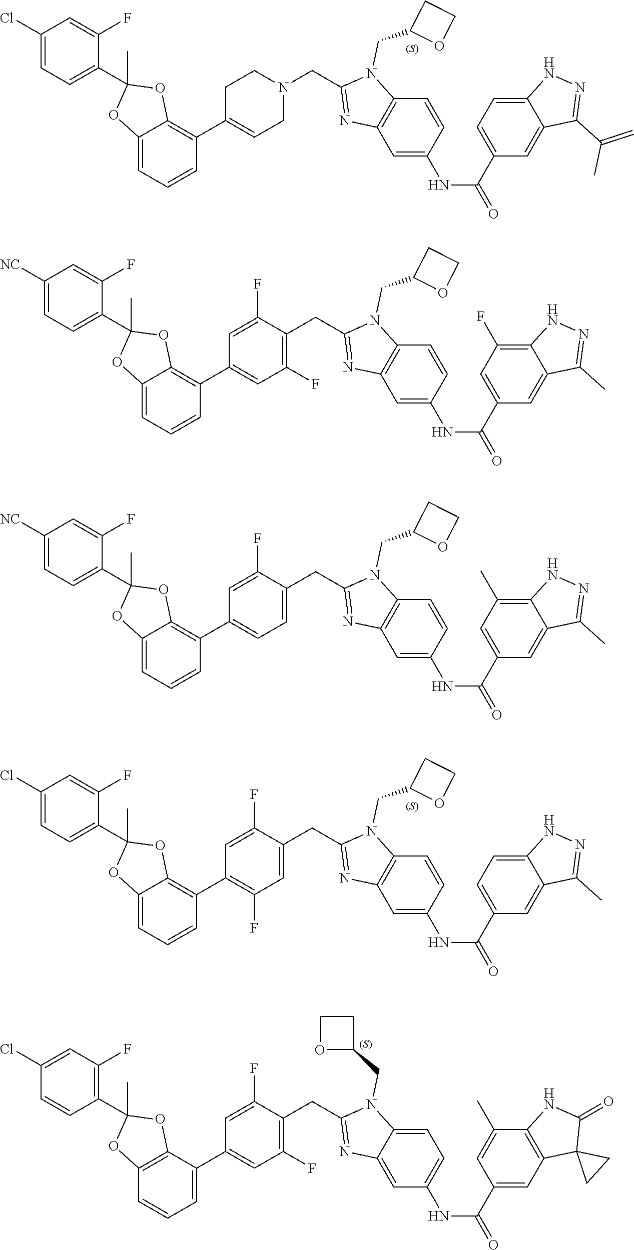

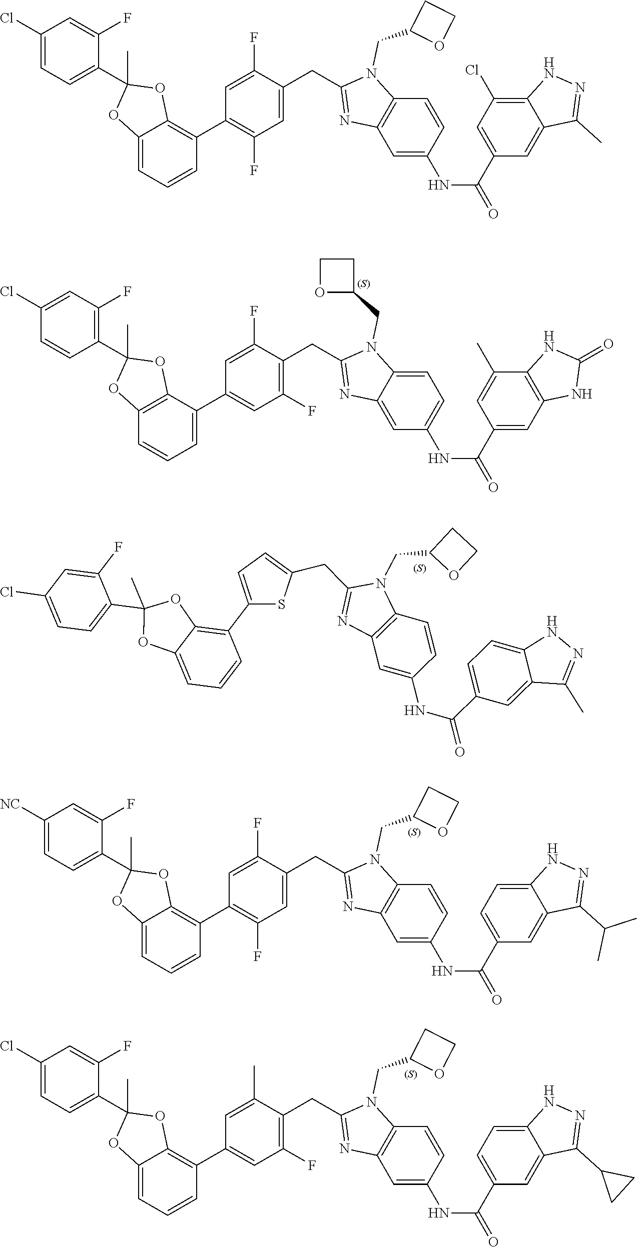

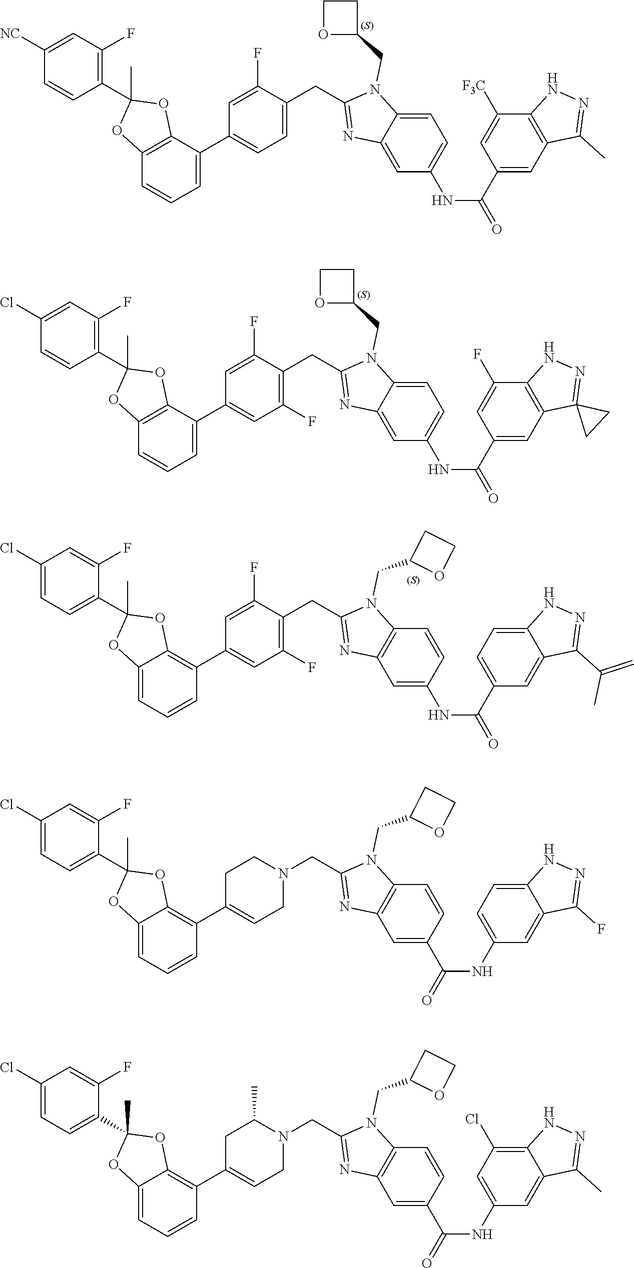

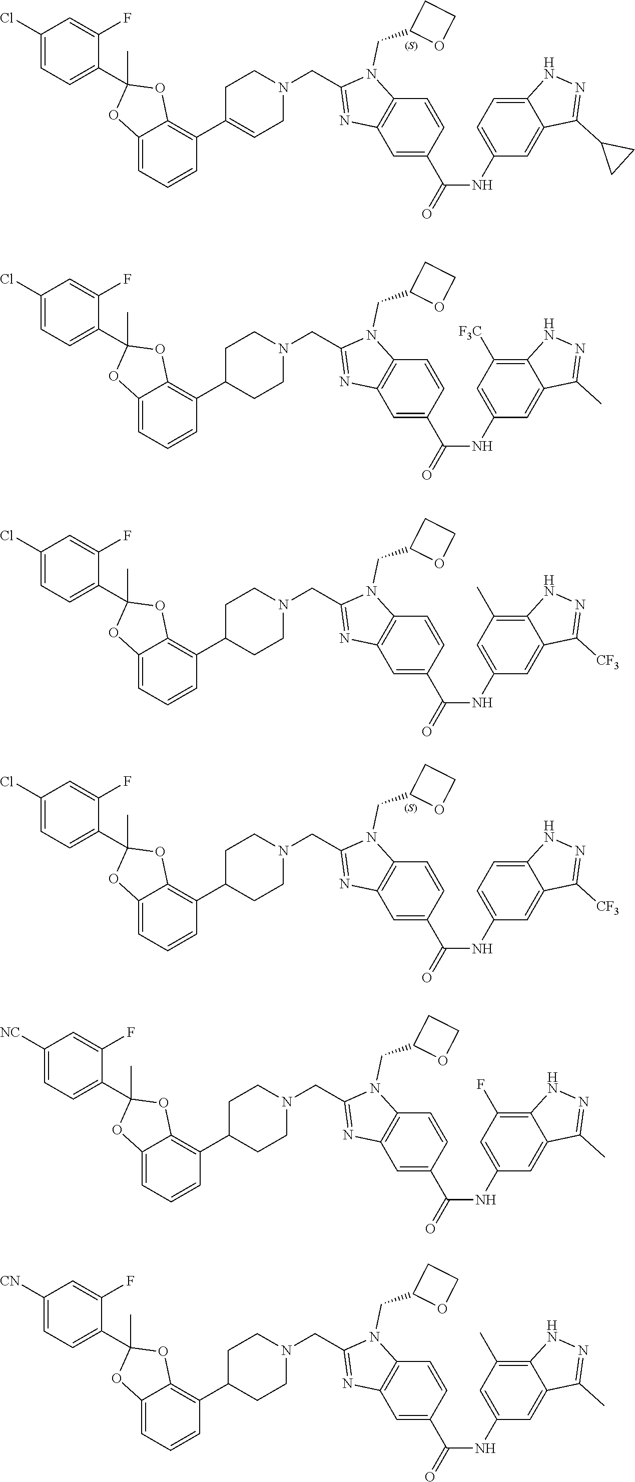

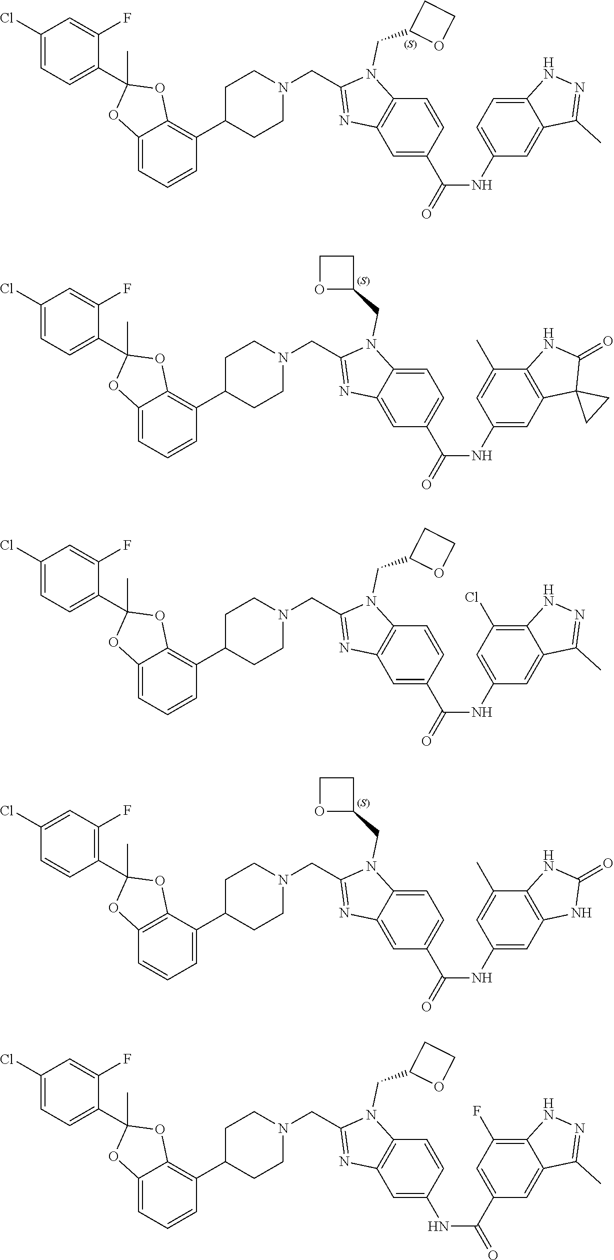

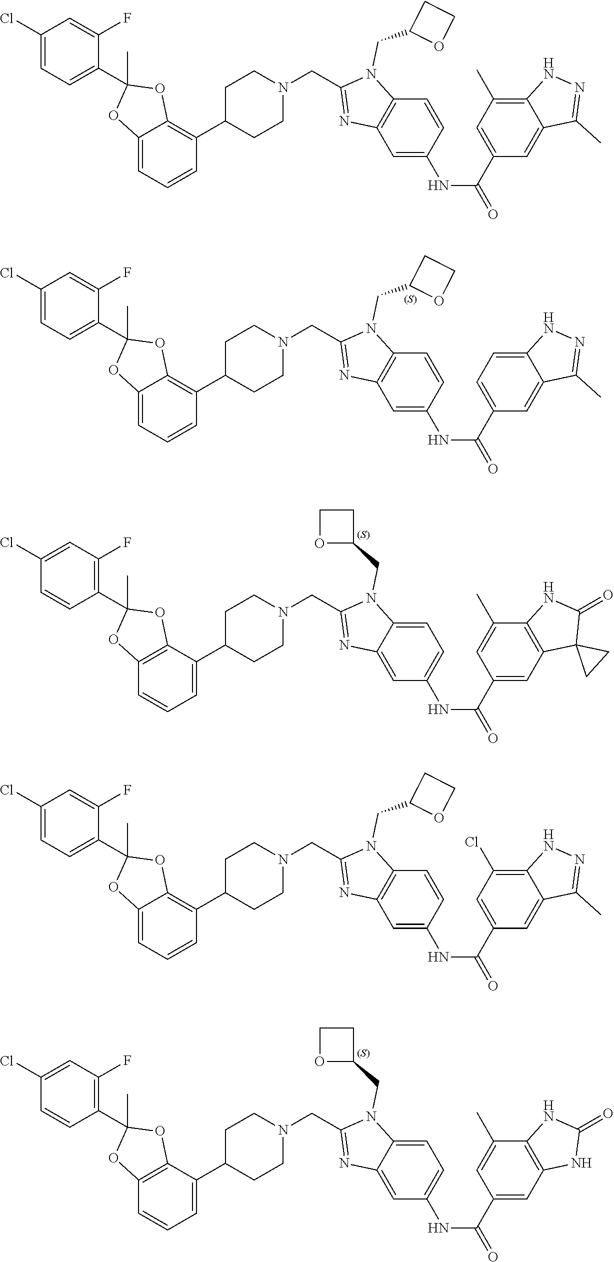

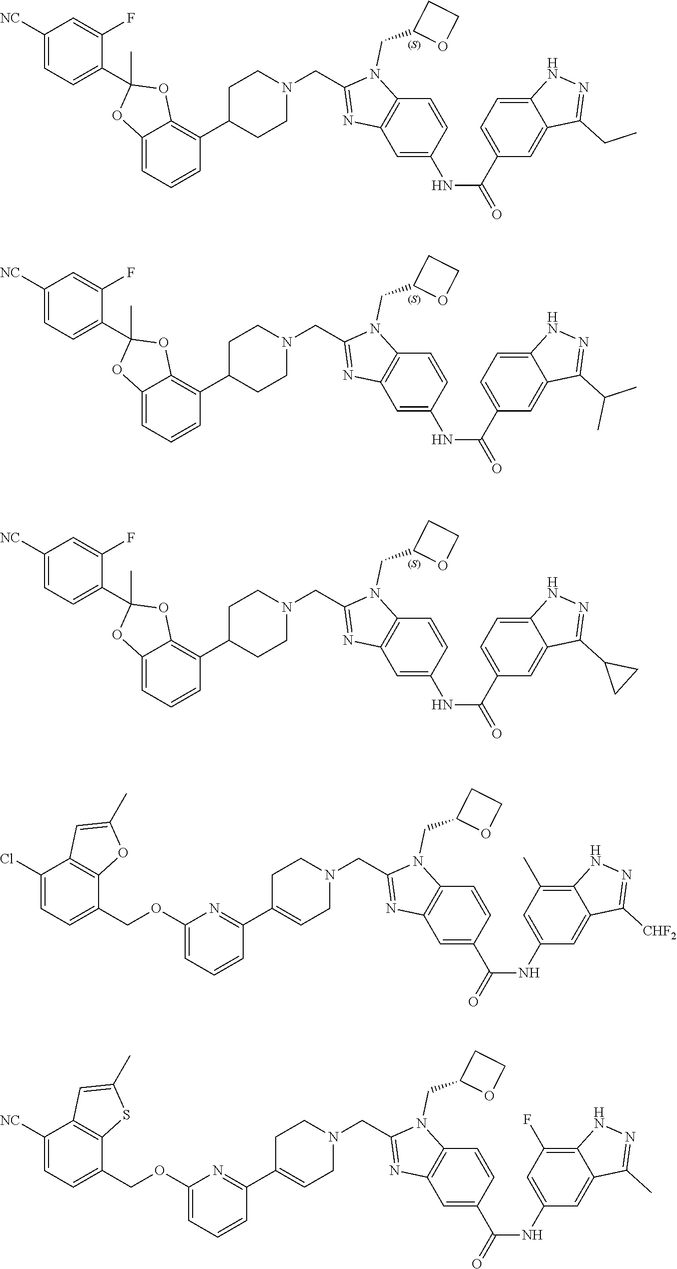

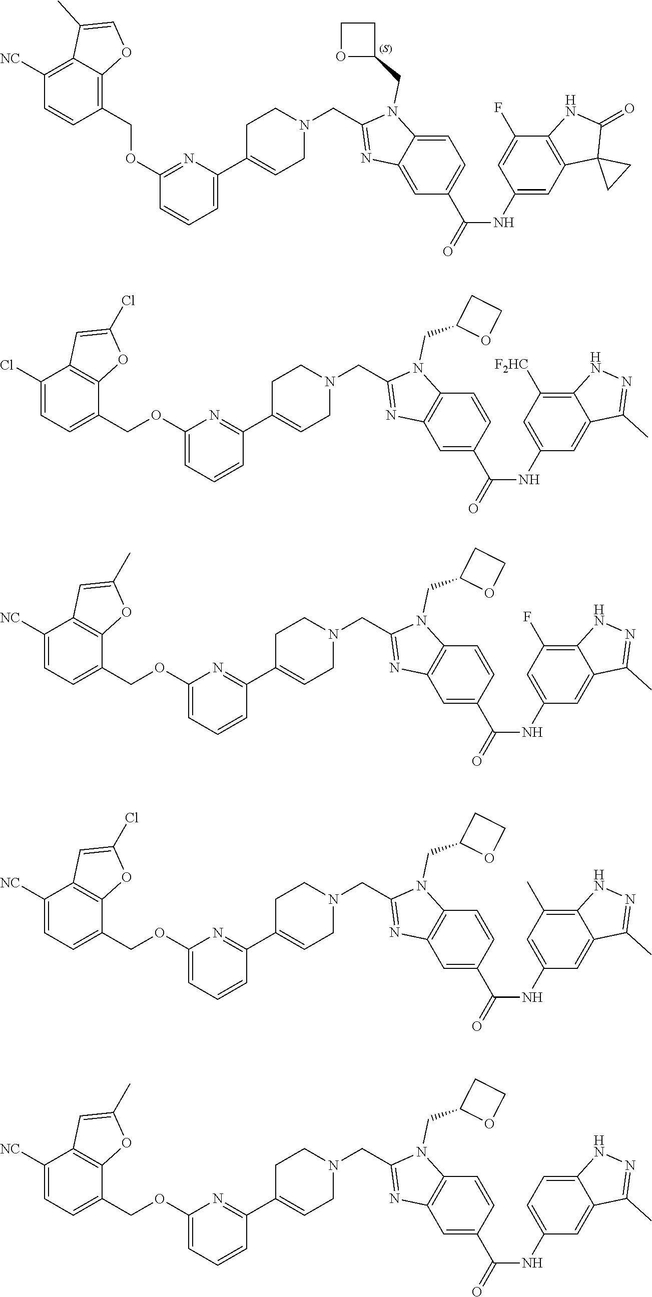

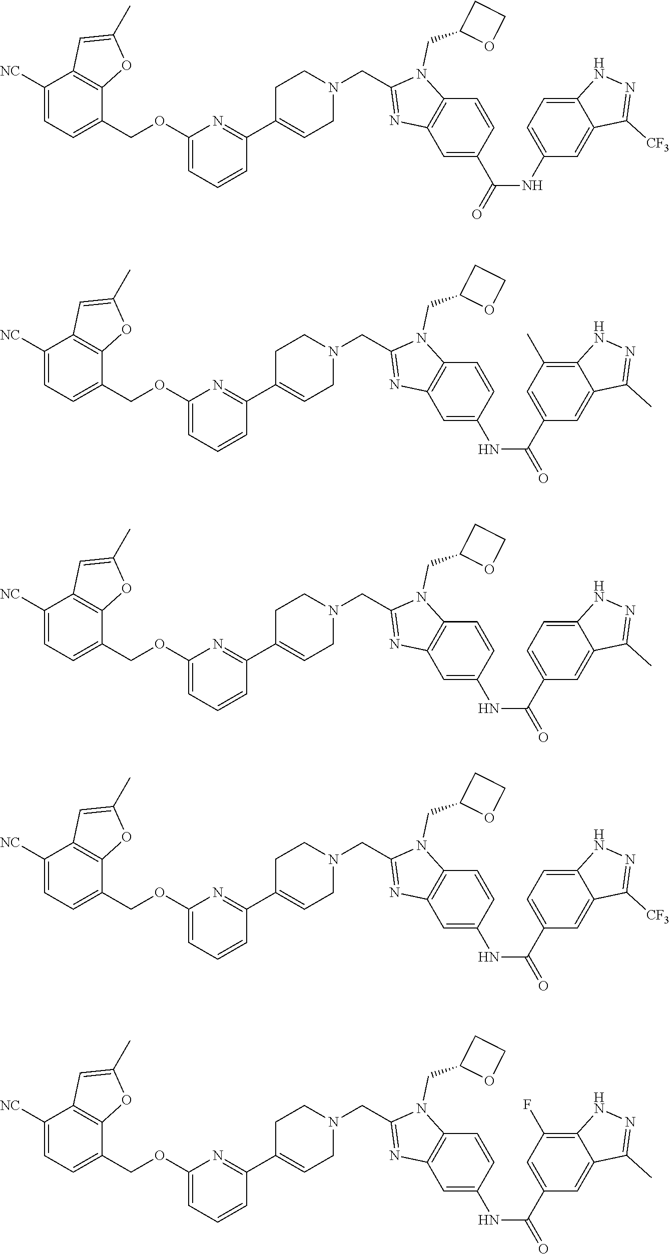

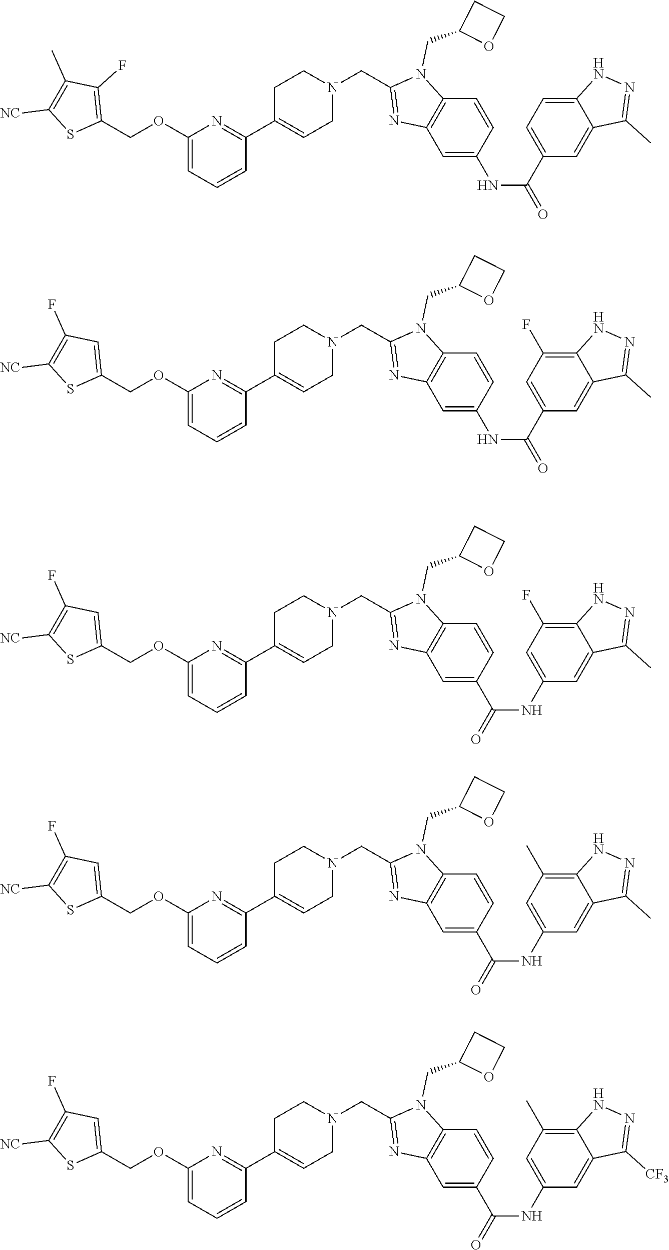

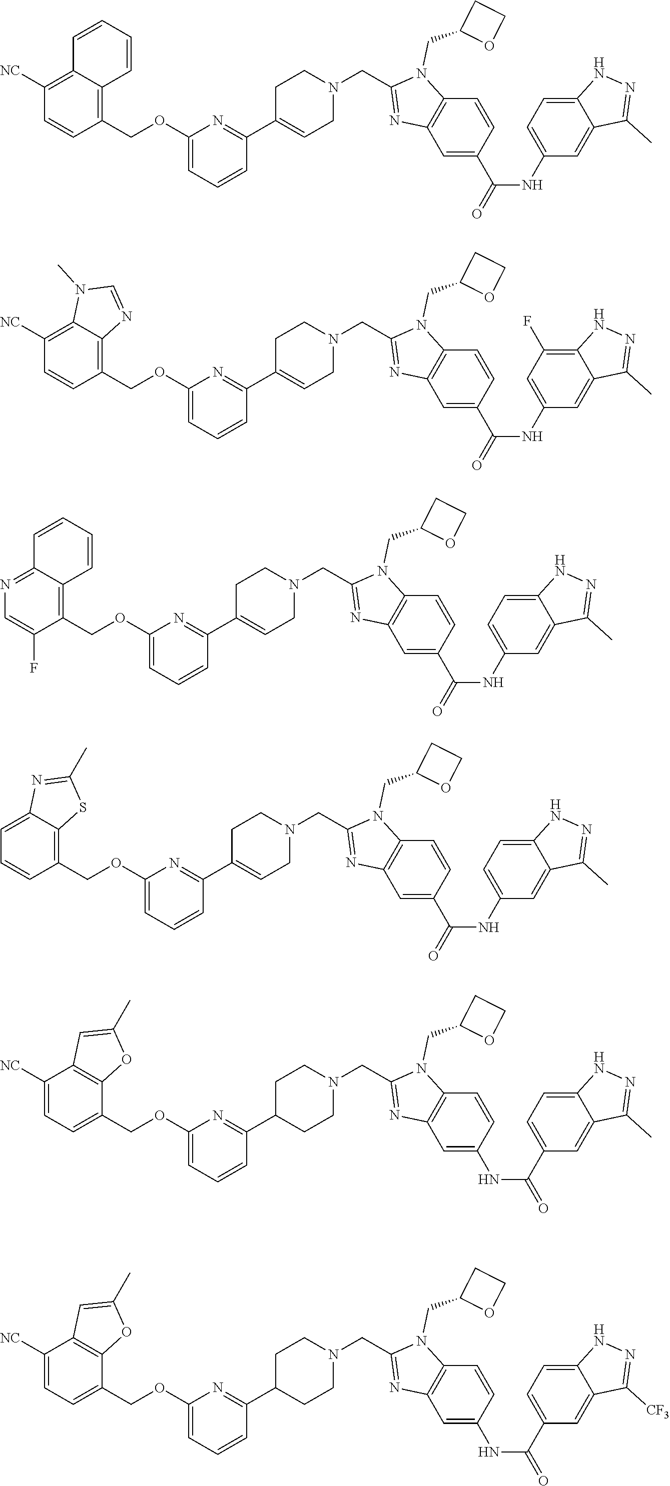

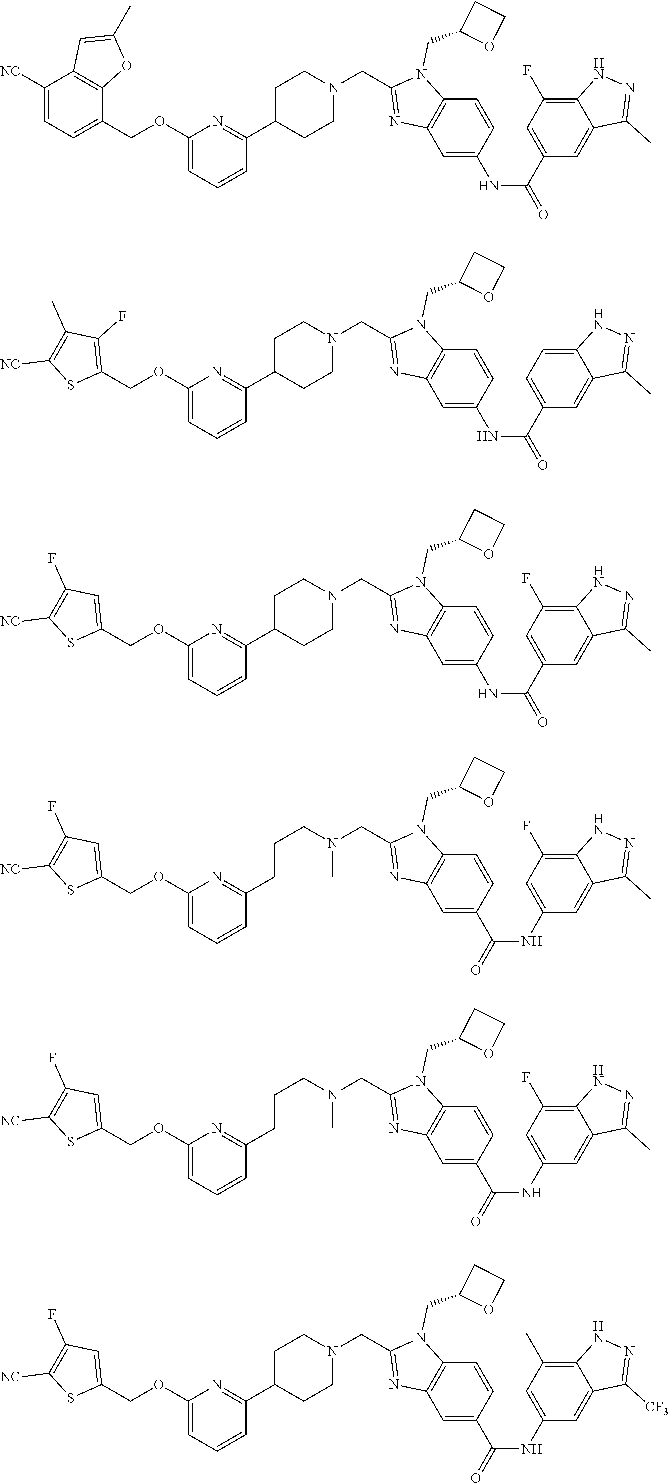

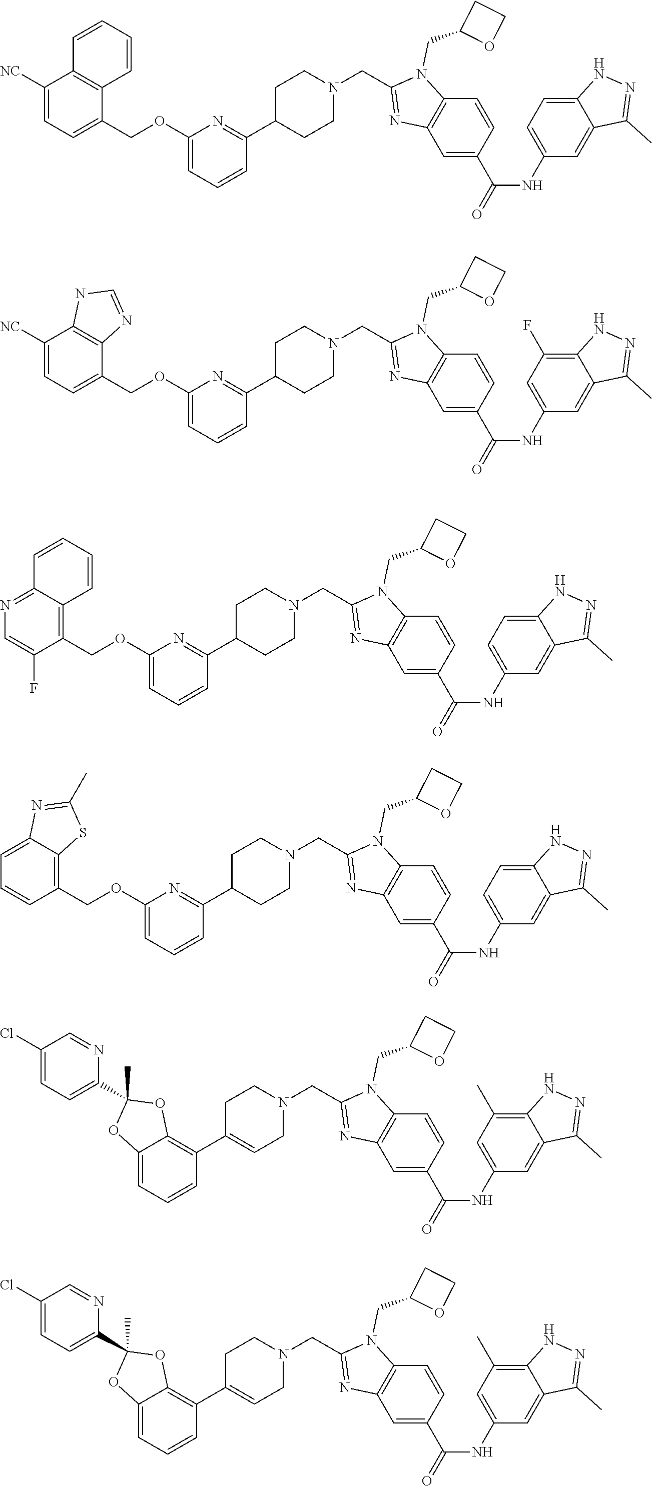

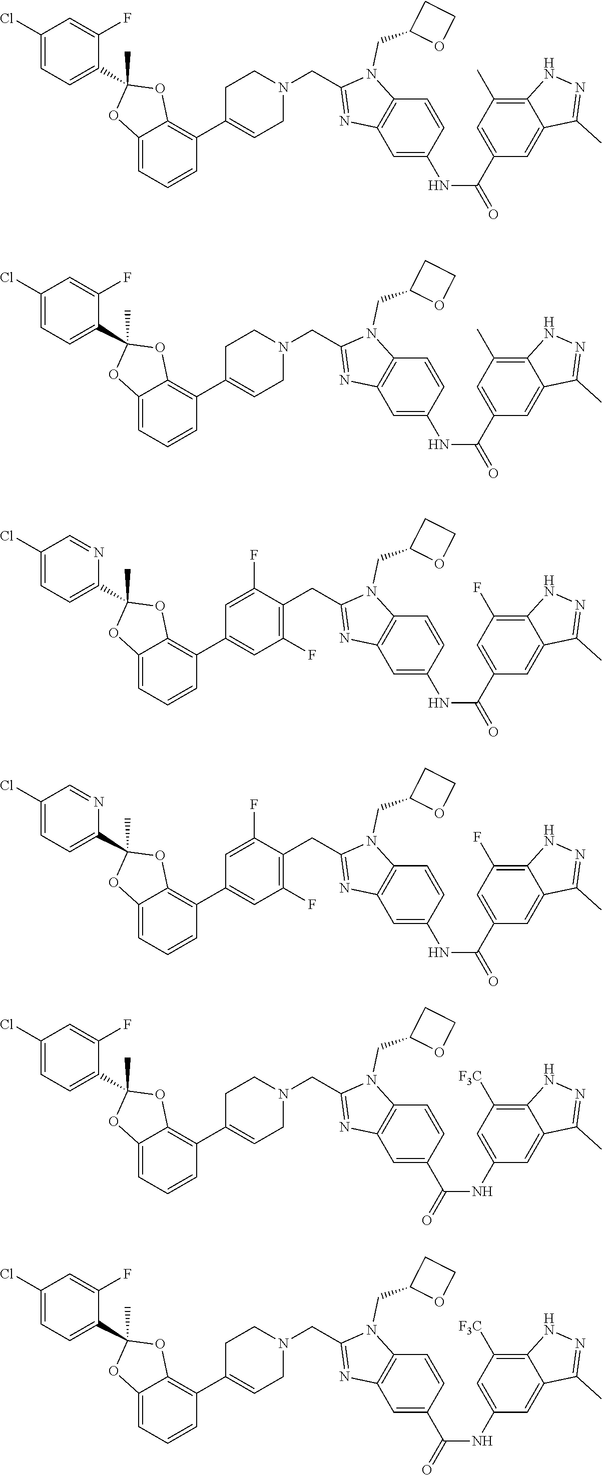

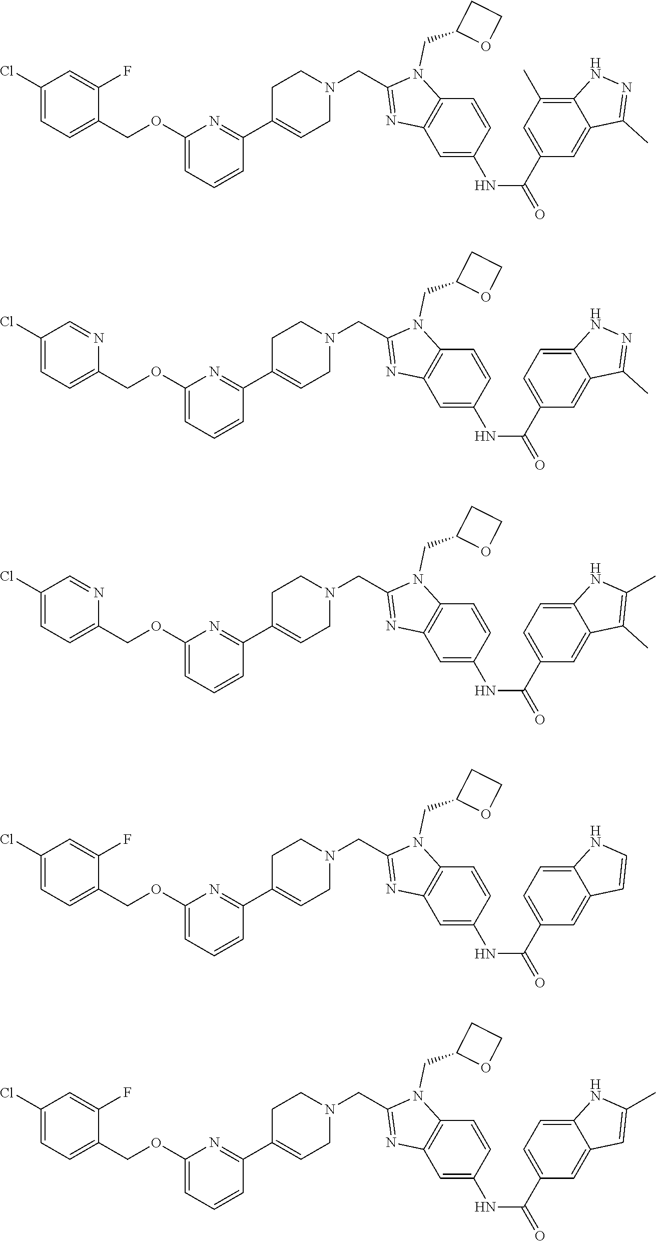 [SMILES: Cc1n[nH]c2c(C)cc(C(=O)Nc3ccc4c(c3)nc(CN3CC=C(c5cccc(OCc6ccc(Cl)cc6F)n5)CC3)n4C[C@@H]3CCO3)cc12 ; Cc1n[nH]c2ccc(C(=O)Nc3ccc4c(c3)nc(CN3CC=C(c5cccc(OCc6ccc(Cl)cn6)n5)CC3)n4C[C@@H]3CCO3)cc12 ; Cc1[nH]c2ccc(C(=O)Nc3ccc4c(c3)nc(CN3CC=C(c5cccc(OCc6ccc(Cl)cn6)n5)CC3)n4C[C@@H]3CCO3)cc2c1C ; O=C(Nc1ccc2c(c1)nc(CN1CC=C(c3cccc(OCc4ccc(Cl)cc4F)n3)CC1)n2C[C@@H]1CCO1)c1ccc2[nH]ccc2c1 ; Cc1cc2cc(C(=O)Nc3ccc4c(c3)nc(CN3CC=C(c5cccc(OCc6ccc(Cl)cc6F)n5)CC3)n4C[C@@H]3CCO3)ccc2[nH]1]

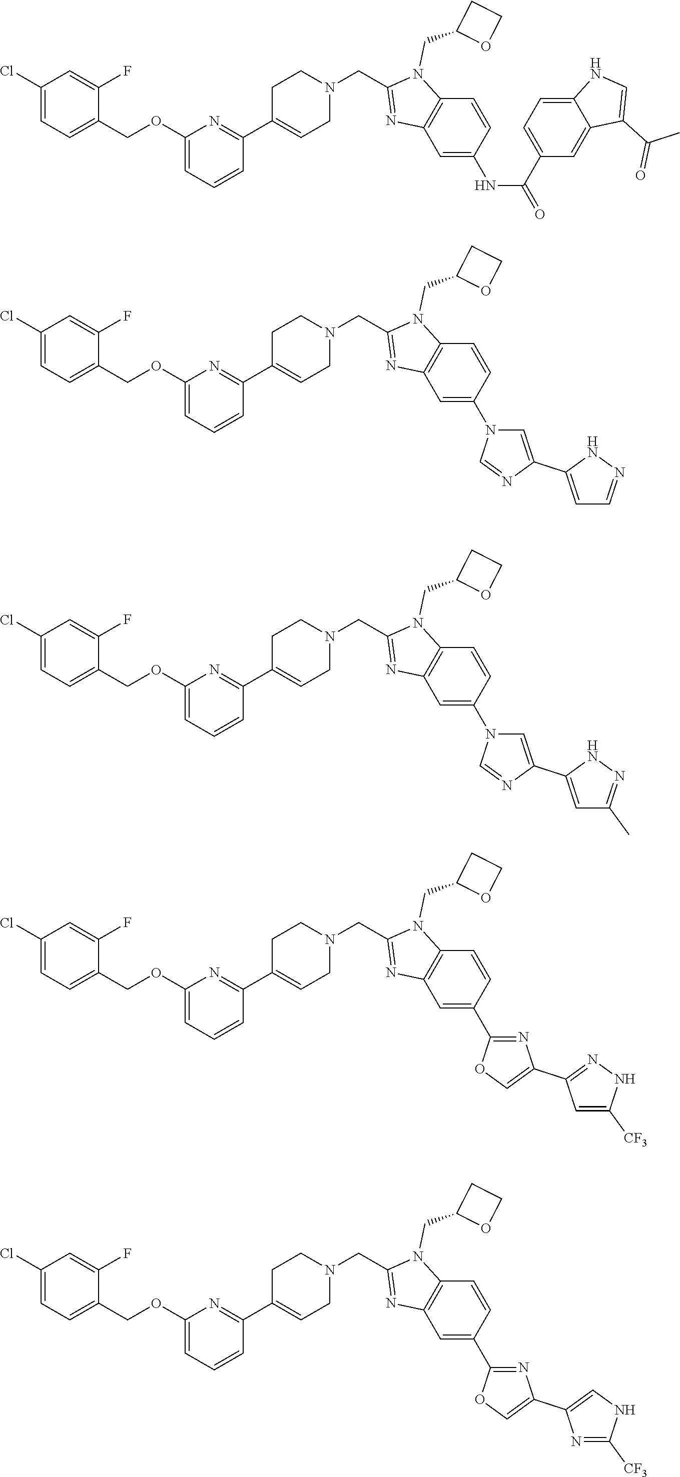 [SMILES: CC(=O)c1c[nH]c2ccc(C(=O)Nc3ccc4c(c3)nc(CN3CC=C(c5cccc(OCc6ccc(Cl)cc6F)n5)CC3)n4C[C@@H]3CCO3)cc12 ; Fc1cc(Cl)ccc1COc1cccc(C2=CCN(Cc3nc4cc(-n5cnc(-c6ccn[nH]6)c5)ccc4n3C[C@@H]3CCO3)CC2)n1 ; Cc1cc(-c2cn(-c3ccc4c(c3)nc(CN3CC=C(c5cccc(OCc6ccc(Cl)cc6F)n5)CC3)n4C[C@@H]3CCO3)cn2)[nH]n1 ; Fc1cc(Cl)ccc1COc1cccc(C2=CCN(Cc3nc4cc(-c5nc(-c6cc(C(F)(F)F)[nH]n6)co5)ccc4n3C[C@@H]3CCO3)CC2)n1 ; Fc1cc(Cl)ccc1COc1cccc(C2=CCN(Cc3nc4cc(-c5nc(-c6c[nH]c(C(F)(F)F)n6)co5)ccc4n3C[C@@H]3CCO3)CC2)n1]

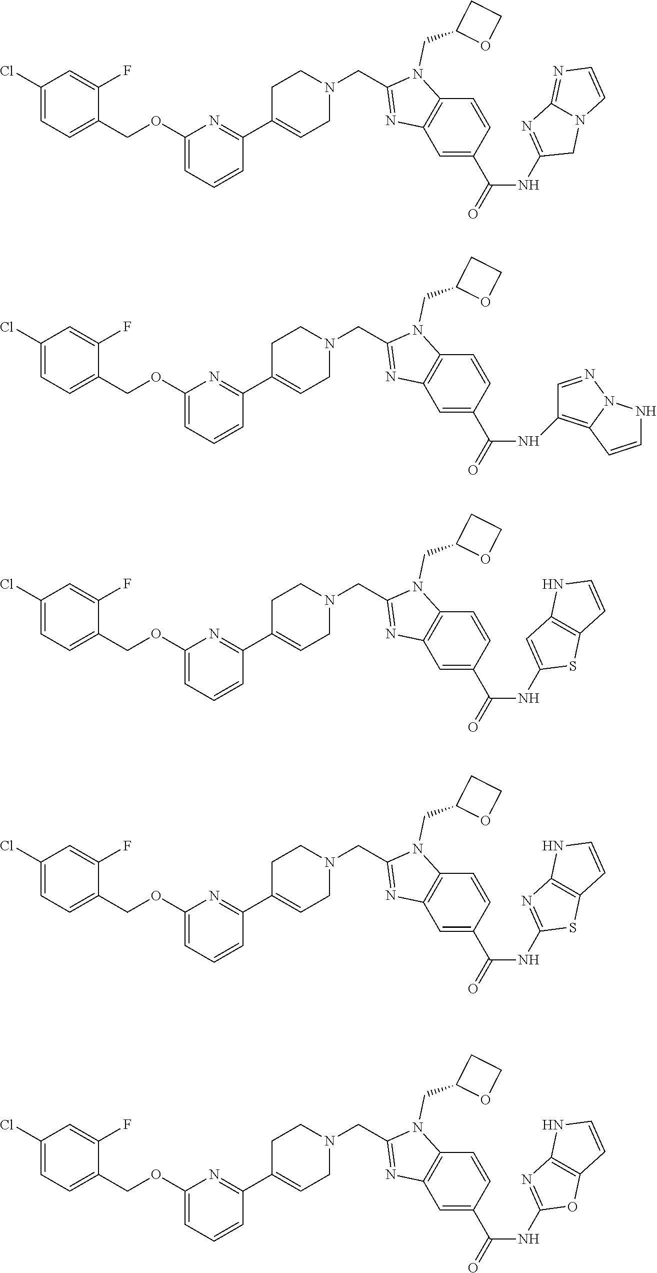

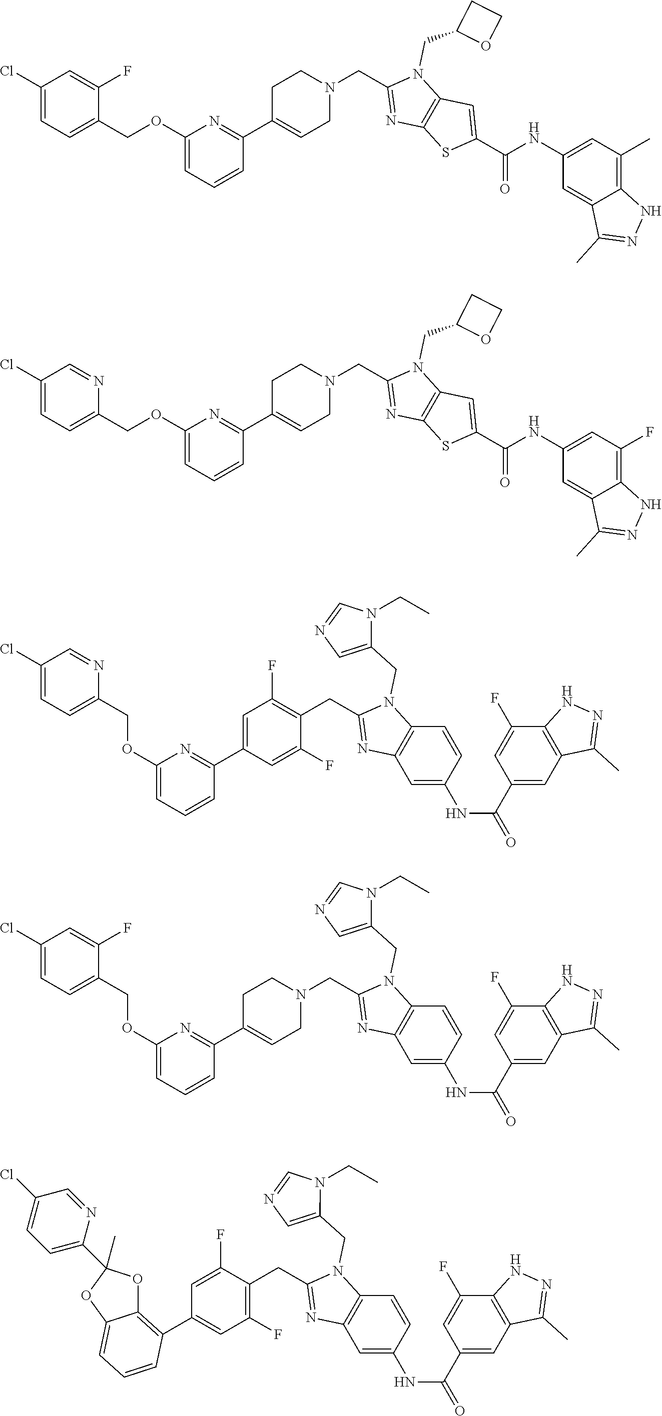 [SMILES: Cc1n[nH]c2c(C)cc(NC(=O)c3cc4c(nc(CN5CC=C(c6cccc(OCc7ccc(Cl)cc7F)n6)CC5)n4C[C@@H]4CCO4)s3)cc12 ; Cc1n[nH]c2c(F)cc(NC(=O)c3cc4c(nc(CN5CC=C(c6cccc(OCc7ccc(Cl)cn7)n6)CC5)n4C[C@@H]4CCO4)s3)cc12 ; CCn1cncc1Cn1c(Cc2c(F)cc(-c3cccc(OCc4ccc(Cl)cn4)n3)cc2F)nc2cc(NC(=O)c3cc(F)c4[nH]nc(C)c4c3)ccc21 ; CCn1cncc1Cn1c(CN2CC=C(c3cccc(OCc4ccc(Cl)cc4F)n3)CC2)nc2cc(NC(=O)c3cc(F)c4[nH]nc(C)c4c3)ccc21 ; CCn1cncc1Cn1c(Cc2c(F)cc(-c3cccc4c3OC(C)(c3ccc(Cl)cn3)O4)cc2F)nc2cc(NC(=O)c3cc(F)c4[nH]nc(C)c4c3)ccc21]

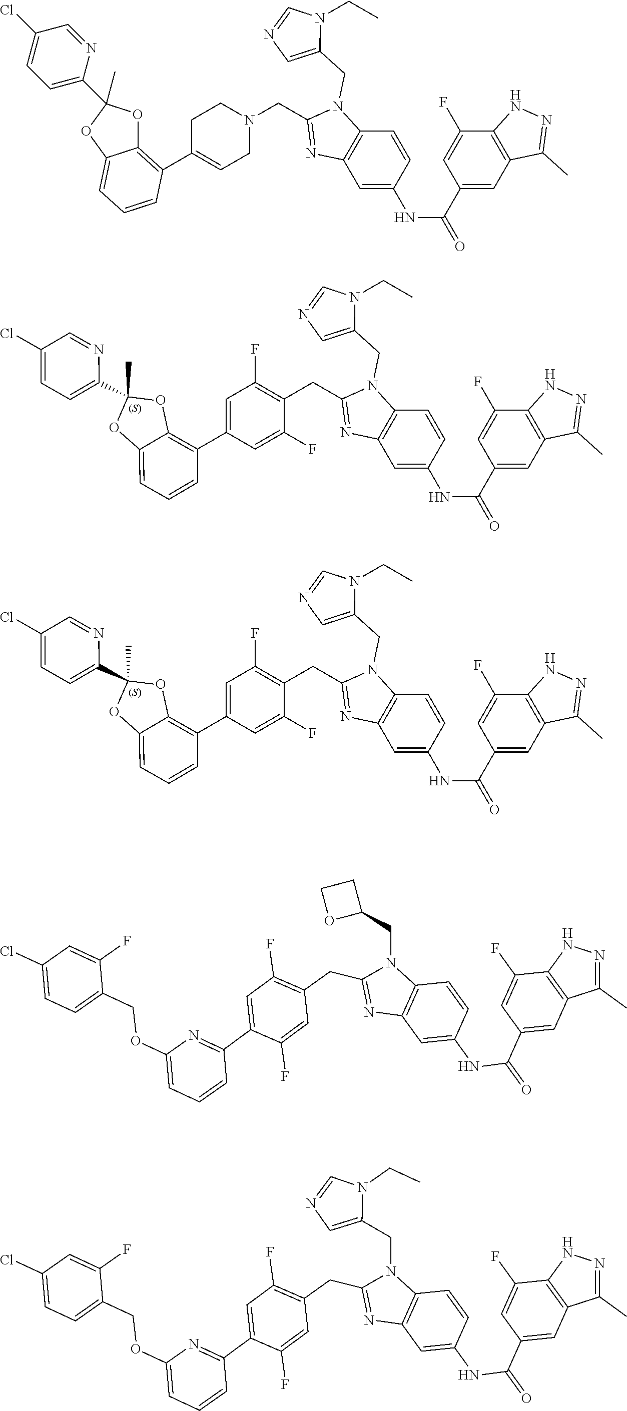

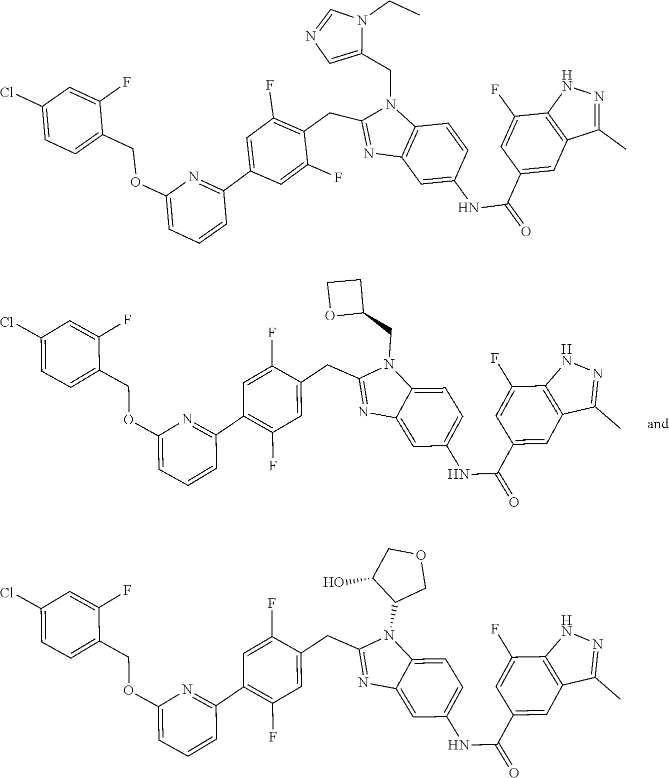

### III. Pharmaceutical Compositions

Another aspect of the present application relates to pharmaceutical compositions that contains the compounds of the present application. In some embodiments, the present application provides a pharmaceutical composition comprising a compound of the present application, and one or more pharmaceutically acceptable excipient.

In some embodiments, the pharmaceutical composition comprises a compound of Formula I-A-1 or a pharmaceutically acceptable salt thereof.

In some embodiments, the pharmaceutical composition comprises a compound of Formula I-A-2 or a pharmaceutically acceptable salt thereof.

In some embodiments, the pharmaceutical composition comprises a compound of Formula I-A-3 or a pharmaceutically acceptable salt thereof.

In some embodiments, the pharmaceutical composition comprises a compound of Formula I-A-4 or a pharmaceutically acceptable salt thereof.

In some embodiments, the pharmaceutical composition comprises a compound of Formula I-A-5 or a pharmaceutically acceptable salt thereof.

In some embodiments, the pharmaceutical composition comprises a compound of Formula I-A-6 or a pharmaceutically acceptable salt thereof.

In some embodiments, the pharmaceutical composition comprises a compound of Formula I-A-7 or a pharmaceutically acceptable salt thereof.

In some embodiments, the pharmaceutical composition of the present application further comprises one or more additional therapeutic agents. Examples of the additional therapeutic agents include, but are not limited to, those therapeutic agents listed in the Combination therapy section of this application.

Pharmaceutical compositions of the present application may be in any form suitable for the intended method of administration. In some embodiments, the pharmaceutical composition is in the form of tablets, sachets, troches, lozenges, aqueous or oil suspensions, dispersible powders or granules, emulsions, hard or soft capsules, syrups or elixirs for oral administration.

In some embodiments, the pharmaceutical compositions of the present application are presented in unit dosage form, including but not limited to, capsules, sachets or tablets each containing a predetermined amount of the active ingredient. In one embodiment, the pharmaceutical composition is a tablet.

The pharmaceutical composition may be prepared by any of the methods well known in the art of pharmacy. Such methods include the step of bringing into association the active ingredient (e.g., a compound of the present application or a pharmaceutical salt thereof) with the one or more pharmaceutically acceptable excipients. In some embodiments, the pharmaceutical compositions are prepared by uniformly and intimately bringing into association the active ingredient with liquid excipients or finely divided solid excipients or both, and then, if desired, shaping the product. Techniques and formulations generally are found in Remington: The Science and Practice of Pharmacy, 21st Edition, Lippincott Williams and Wilkins, Philadelphia, Pa., 2006.

Examples of the one or more excepients include, but not limited to, fillers, binders, bulking agent, glidants, sweetening agents, flavoring agents, coloring agents and preserving agents. In some embodiments, the pharmaceutical composition is in tablet form and comprises one or more pharmaceutically acceptable excipients which are suitable for manufacture of tablets. These excipients may be, for example, inert diluents, such as calcium or sodium carbonate, lactose, lactose monohydrate, croscarmellose sodium, povidone, calcium or sodium phosphate; granulating and disintegrating agents, such as maize starch, or alginic acid; binding agents, such as cellulose, microcrystalline cellulose, starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

The amount of active ingredient that may be combined with the inactive ingredients to produce a dosage form may vary depending upon the intended treatment subject and the mode of administration. For example, in some embodiments, a dosage form for oral administration to humans may contain approximately 1 to 1000 mg of active material formulated with an appropriate and convenient amount of a pharmaceutically acceptable excipient. In some embodiments, the pharmaceutically acceptable excipient varies from about 5% to about 95% of the total compositions (weight:weight).

In some embodiments, the pharmaceutical composition of the present application does not contain an agent that affects the rate at which the active ingredient is metabolized. Thus, it is understood that pharmaceutical compositions comprising a compound of the present application in one aspect do not comprise an agent that would affect (e.g., slow, hinder or retard) the metabolism of a compound of the present application or any other active ingredient administered separately, sequentially or simultaneously with a compound of the present application. It is also understood that any of the methods, kits, articles of manufacture and the like detailed herein in one aspect do not comprise an agent that would affect (e.g., slow, hinder or retard) the metabolism of a compound of the present application or any other active ingredient administered separately, sequentially or simultaneously with a compound of the present application.

### IV. Methods of Treatment

Another aspect of the present application relates to a method for preventing, treating or ameliorating a symptom of a GLP-1R-mediated disease or condition with the compound of the present application. In some embodiments, the method comprises the step of administering to a subject in need of such treatment an effective amount of a compound of Formula I-A-1, I-A-2, I-A-3, I-A-4, I-A-5, I-A-6, I-A-7, or a pharmaceutically acceptable salt thereof.

Examples of GLP-1R-mediated diseases or conditions include, but are not limited to, present application chronic intrahepatic or some forms extrahepatic cholestatic conditions, liver fibrosis, acute intrahepatic cholestatic conditions, obstructive or chronic inflammatory disorders that arise out of improper bile composition, gastrointestinal conditions with a reduced uptake of dietary fat and fat-soluble dietary vitamins, inflammatory bowel diseases, lipid and lipoprotein disorders, type II diabetes and clinical complications of type I and type II diabetes, conditions and diseases which result from chronic fatty and fibrotic degeneration of organs due to enforced lipid and specifically triglyceride accumulation and subsequent activation of profibrotic pathways, of obesity and metabolic syndrome (combined conditions of dyslipidemia, diabetes and abnormally high body-mass index), acute myocardial infarction, acute stroke, thrombosis which occurs as an endpoint of chronic obstructive atherosclerosis, persistent infections by intracellular bacteria or parasitic protozoae, non-malignant hyperproliferative disorders, malignant hyperproliferative disorders, colon adenocarcinoma and hepatocellular carcinoma for instance, liver steatosis and associated syndromes, liver failure or liver malfunction as an outcome of chronic liver diseases or of surgical liver resection, Hepatitis B infection, Hepatitis C infection and/or of cholestatic and fibrotic effects that are associated with alcohol-induced cirrhosis or with viral-borne forms of hepatitis, type I diabetes, pre-diabetes, idiopathic type 1 diabetes, latent autoimmune diabetes, maturity onset diabetes of the young, early onset diabetes, malnutrition-related diabetes, gestational diabetes, hyperglycemia, insulin resistance, hepatic insulin resistance, impaired glucose tolerance, diabetic neuropathy, diabetic nephropathy, kidney disease, diabetic retinopathy, adipocyte dysfunction, visceral adipose deposition, obesity, eating disorders, sleep apnea, weight gain, sugar craving, dyslipidemia, hyperinsulinemia, congestive heart failure, myocardial infarction, stroke, hemorrhagic stroke, ischemic stroke, traumatic brain injury, pulmonary hypertension, restenosis after angioplasty, intermittent claudication, post-prandial lipemia, metabolic acidosis, ketosis, arthritis, left ventricular hypertrophy, Parkinson's Disease, peripheral arterial disease, macular degeneration, cataract, glomerulosclerosis, chronic renal failure, metabolic syndrome, angina pectoris, premenstrual syndrome, thrombosis, atherosclerosis, impaired glucose metabolism, or vascular restenosis.

In some embodiments, the GLP-1R-mediated disease or condition is selected from the group consisting of type I diabetes (T1D), type II diabetes mellitus (T2DM), pre-diabetes, idiopathic T1D, late-onset autoimmune diabetes of adulthood (LADA), early-onset T2DM (EOD), youth-onset atypical diabetes (YOAD), maturity-onset diabetes of the young (MODY), malnutrition-related diabetes, gestational diabetes, hyperglycemia, insulin resistance, hepatic insulin resistance, impaired glucose tolerance, diabetic neuropathy, diabetic nephropathy, kidney disease, diabetic retinopathy, adipocyte dysfunction, visceral adipose deposition, sleep apnea, obesity, eating disorders, weight gain from use of other agents, excessive sugar craving, dyslipidemia, hyperinsulinemia, NAFLD, NASH, fibrosis, cirrhosis, hepatocellular carcinoma, cardiovascular disease, atherosclerosis, coronary artery disease, peripheral vascular disease, hypertension, endothelial dysfunction, impaired vascular compliance, congestive heart failure, myocardial infarction, stroke, hemorrhagic stroke, ischemic stroke, traumatic brain injury, pulmonary hypertension, restenosis after angioplasty, intermittent claudication, post-prandial lipemia, metabolic acidosis, ketosis, arthritis, osteoporosis, Parkinson's Disease, left ventricular hypertrophy, peripheral arterial disease, macular degeneration, cataract, glomerulosclerosis, chronic renal failure, metabolic syndrome, syndrome X, premenstrual syndrome, angina pectoris, thrombosis, atherosclerosis, transient ischemic attacks, vascular restenosis, impaired glucose metabolism, conditions of impaired fasting plasma glucose, hyperuricemia, gout, erectile dysfunction, skin and connective tissue disorders, psoriasis, foot ulcerations, ulcerative colitis, hyper apo B lipoproteinemia, Alzheimer's Disease, schizophrenia, impaired cognition, inflammatory bowel disease, short bowel syndrome, Crohn's disease, colitis, irritable bowel syndrome, prevention or treatment of Polycystic Ovary Syndrome and treatment of addiction.

In some embodiments, the GLP-1R-mediated disease or condition is a liver disease, e.g., liver fibrosis, non-alcoholic fatty liver disease (NAFLD), non-alcoholic steatohepatitis (NASH), liver cirrhosis, compensated liver fibrosis, decompensated liver fibrosis, hepatocellular carcinoma, Primary Biliary Cirrhosis (PBC), or Primary Sclerosing Cholangitis (PSC).

In some embodiments, the GLP-1R-mediated disease or condition is a metabolic disease.

In some embodiments, the present application relates to a method for treating and/or preventing NAFLD, comprises administering to a subject in need thereof n effective amount of a compound of Formula I-A-1 or a pharmaceutically acceptable salt thereof.

In some embodiments, the present application relates to a method for treating and/or preventing NASH. In some embodiments, the method comprises administering an effective amount of a compound of Formula I-A-1 to a subject in need thereof.

In some embodiments, the present application relates to preventive and posttraumatic treatment of a cardiovascular disorder, such as acute myocardial infarction, acute stroke, or thrombosis which occur as an endpoint of chronic obstructive atherosclerosis. In some embodiments, the method comprises administering an effective amount of a compound of Formula I-A-1, to a subject in need thereof.

In some embodiments, the present application relates to a method for the treatment and/or prevention of obesity and associated disorders such as metabolic syndrome (combined conditions of dyslipidemias, diabetes and abnormally high body-mass index), which can be overcome by GLP1R-mediated lowering of serum triglycerides, blood glucose and increased insulin sensitivity and GLP1R-mediated weight loss. In some embodiments, the method comprises administering an effective amount of a compound of Formula I-A-1 to a subject in need thereof.

In some embodiments, the present application relates to a method for preventing and/or treating clinical complications of Type I and Type II Diabetes. Examples of such complications include diabetic nephropathy, diabetic retinopathy, diabetic neuropathies, or Peripheral Arterial Occlusive Disease (PAOD). Other clinical complications of diabetes are also encompassed by the present application. In some embodiments, the method comprises administering an effective amount of a compound of Formula I-A-1 to a subject in need thereof.

Furthermore, conditions and diseases which result from chronic fatty and fibrotic degeneration of organs due to enforced lipid and/or triglyceride accumulation and subsequent activation of profibrotic pathways may also be prevented and/or treated by administering the compounds or pharmaceutical compositions of the present application. Such conditions and diseases include, but are not limited to, NASH and chronic cholestatic conditions in the liver, Glomerulosclerosis and Diabetic Nephropathy in the kidney, Macular Degeneration and Diabetic Retinopathy in the eye and neurodegenerative diseases, such as Alzheimer's Disease in the brain, or Diabetic Neuropathies in the peripheral nervous system.

In some embodiments, the present application relates to a method for treating and/or preventing conditions and diseases which result from chronic fatty and fibrotic degeneration of organs due to enforced lipid and/or triglyceride accumulation and subsequent activation of profibrotic pathways. In some embodiments, the method comprises administering an effective amount of a compound of Formula I-A-1 to a subject in need thereof.

In some embodiments, the compound of the present application is used in the form of a prodrug or other suitably modified form, which releases the active ingredient in vivo.

The compounds of the present application (also referred to herein as the active ingredients) or pharmaceutical compositions of the present application can be administered by any route appropriate to the condition to be treated. Suitable routes include oral, rectal, nasal, topical (including buccal and sublingual), transdermal, vaginal and parenteral (including subcutaneous, intramuscular, intravenous, intradermal, intratumoral, intrathecal and epidural), and the like. It will be appreciated that the preferred route may vary with for example the condition of the recipient. An advantage of certain compounds disclosed herein is that they are orally bioavailable and can be dosed orally.

A compound of the present application may be administered to an individual in accordance with an effective dosing regimen for a desired period of time or duration, such as at least about one month, at least about 2 months, at least about 3 months, at least about 6 months, or at least about 12 months or longer. In one variation, the compound is administered on a daily or intermittent schedule for the duration of the individual's life.

The dosage or dosing frequency of a compound of the present application may be adjusted over the course of the treatment, based on the judgment of the administering physician.

The compound may be administered to an individual (e.g., a human) in an effective amount. In some embodiments, the compound is administered once daily.

The compound can be administered by any useful route and means, such as by oral or parenteral (e.g., intravenous) administration. Therapeutically effective amounts of the compound may include from about 0.00001 mg/kg body weight per day to about 10 mg/kg body weight per day, such as from about 0.0001 mg/kg body weight per day to about 10 mg/kg body weight per day, or such as from about 0.001 mg/kg body weight per day to about 1 mg/kg body weight per day, or such as from about 0.01 mg/kg body weight per day to about 1 mg/kg body weight per day, or such as from about 0.05 mg/kg body weight per day to about 0.5 mg/kg body weight per day, or such as from about 0.3 mg to about 30 mg per day, or such as from about 30 mg to about 300 mg per day.

A compound of the present application may be combined with one or more additional therapeutic agents in any dosage amount of the compound of the present application (e.g., from 1 mg to 1000 mg of compound). Therapeutically effective amounts may include from about 1 mg per dose to about 1000 mg per dose, such as from about 50 mg per dose to about 500 mg per dose, or such as from about 100 mg per dose to about 400 mg per dose, or such as from about 150 mg per dose to about 350 mg per dose, or such as from about 200 mg per dose to about 300 mg per dose. Other therapeutically effective amounts of the compound of the present application are about 100, 125, 150, 175, 200, 225, 250, 275, 300, 325, 350, 375, 400, 425, 450, 475, or about 500 mg per dose. Other therapeutically effective amounts of the compound of the present application are about 100 mg per dose, or about 125, 150, 175, 200, 225, 250, 275, 300, 350, 400, 450, or about 500 mg per dose. A single dose can be administered hourly, daily, or weekly. For example, a single dose can be administered once every 1 hour, 2, 3, 4, 6, 8, 12, 16 or once every 24 hours. A single dose can also be administered once every 1 day, 2, 3, 4, 5, 6, or once every 7 days. A single dose can also be administered once every 1 week, 2, 3, or once every 4 weeks. In some embodiments, a single dose can be administered once every week. A single dose can also be administered once every month.

Kits that comprise a compound of the present application, or pharmaceutically acceptable salt thereof, are also included in the present application. In one embodiment, a kit further includes a label and/or instructions for use of the compounds in the treatment of the indications, such as the diseases or conditions described herein. In one embodiment, the kit comprises a compound of the present application, or pharmaceutically acceptable salt thereof, in combination with one or more (e.g., one, two, three, four, one or two, or one to three, or one to four) additional therapeutic agents.

### V. Combination Therapy

Also provided herein is the use of a compound of the present application in combination with one or more additional therapeutic agents having a synergistic effect with the compound of the present application.

In some embodiments, the one or more additional therapeutic agents comprise an apoptotic signal-regulating kinase (ASK-1) inhibitor, a farnesoid X receptor (FXR) agonist, a peroxisome proliferator-activated receptor alpha (PPARα) agonist, fish oil, an acetyl-coA carboxylase (ACC) inhibitor, or a TGFβ antagonist, or a combination thereof.

In some embodiments, the one or more additional therapeutic agents are selected from the group consisting of ACE inhibitors, 2-acylglycerol O-acyltransferase 2 (DGAT2) inhibitors, acetaldehyde dehydrogenase inhibitors, acetyl CoA carboxylase inhibitors, adrenergic receptor agonists, Alstrom syndrome protein 1 (ALMS1)/PKC alpha protein interaction inhibitors, apelin receptor agonists, diacylglycerol O acyltransferase 2 inhibitors, adenosine A3 receptor agonists, adenosine A3 receptor antagonists, adiponectin receptor agonists, aldehyde dehydrogenase 2 stimulators, AKT protein kinase inhibitors, AMP-activated protein kinases (AMPK), AMP kinase activators, ATP citrate lyase inhibitors, AMP activated protein kinase stimulators, endothelial nitric oxide synthase stimulators, NAD-dependent deacetylase sirtuin-1 stimulators, androgen receptor agonists, amylin receptor agonists, angiotensin II AT-1 receptor antagonists, autophagy protein modulators, autotaxin inhibitors, Axl tyrosine kinase receptor inhibitors, Bax protein stimulators, beta-catenin inhibitors, bioactive lipids, aalcitonin agonists, cannabinoid receptor modulators, Caspase inhibitors, Caspase-3 stimulators, cathepsin inhibitors, caveolin 1 inhibitors, CCK receptor antagonists, CCL26 gene inhibitors, CCR2 chemokine antagonists, CCR2 chemokine antagonists, angiotensin II AT-1 receptor antagonists, CCR3 chemokine antagonists, CCR5 chemokine antagonists, CD3 antagonists, CDGSH iron sulfur domain protein modulators, chitinase inhibitors, chloride channel stimulators, chitotriosidase 1 inhibitors, CNR1 inhibitors, connective tissue growth factor ligand inhibitors, cyclin D1 inhibitors, cytochrome P4507A1 inhibitors, DGAT1/2 inhibitors, diacylglycerol O acyltransferase 1 (DGAT1) inhibitors, cytochrome P4502E1 (CYP2E1) inhibitors, CXCR4 chemokine antagonists, dihydroceramide delta 4 desaturase inhibitors, dihydroorotate dehydrogenase inhibitors, dipeptidyl peptidase IV inhibitors, endosialin modulators, eotaxin ligand inhibitors, extracellular matrix protein modulators, farnesoid X receptor agonists, fatty acid synthase inhibitors, FGF1 receptor agonists, fibroblast growth factor (FGF-15, FGF-19, FGF-21) ligands, fibroblast activation protein inhibitors, free fatty acid receptor 1 agonists, galectin-3 inhibitors, GDNF family receptor alpha like agonists, glucagon receptor agonists, glucagon-like peptide 1 agonists, glucocorticoid receptor antagonists, glucose 6-phosphate 1-dehydrogenase inhibitors, G-protein coupled bile acid receptor 1 agonists, G-protein coupled receptor-119 agonists, G-protein coupled receptor 84 antagonists, hedgehog (Hh) modulators, hepatitis C virus NS3 protease inhibitors, hepatocyte nuclear factor 4 alpha (HNF4A) modulators, hepatocyte growth factor modulators, histone deacetylase inhibitors, STAT-3 modulators, HMG CoA reductase inhibitors, HSD17B13 gene inhibitors, 5-HT 2a receptor antagonists, hydrolase inhibitors, hypoxia inducible factor-2 alpha inhibitors, IL-10 agonists, IL-17 antagonists, IL-22 agonists, ileal sodium bile acid cotransporter inhibitors, insulin sensitizers, insulin ligand agonists, insulin receptor agonists, integrin modulators, integrin antagonists, integrin alpha-V/beta-1 antagonists, integrin alpha-V/beta-6 antagonists, interleukin-1 receptor-associated kinase 4 (IRAK4) inhibitors, IL-6 receptor agonists, interleukin 17 ligand inhibitors, Jak2 tyrosine kinase inhibitors, Jun N terminal kinase-1 inhibitors, Kelch like ECH associated protein 1 modulators, ketohexokinase (KHK) inhibitors, klotho beta stimulators, leukotriene A4 hydrolase inhibitors, 5-lipoxygenase inhibitors, lipoprotein lipase inhibitors, liver X receptors, LPL gene stimulators, lysophosphatidate-1 receptor antagonists, lysyl oxidase homolog 2 inhibitors, LXR inverse agonists, macrophage mannose receptor 1 modulators, matrix metalloproteinases (MMPs) inhibitors, MEKK-5 protein kinase inhibitors, MCH receptor-1 antagonists, membrane copper amine oxidase (VAP-1) inhibitors, methionine aminopeptidase-2 inhibitors, methyl CpG binding protein 2 modulators, microRNA-132 (miR-132) antagonists, microRNA-21 (miR-21) inhibitors, mitochondrial uncouplers, mixed lineage kinase-3 inhibitors, motile sperm domain protein 2 inhibitors, myelin basic protein stimulators, NACHT LRR PYD domain protein 3 (NLRP3) inhibitors, NAD-dependent deacetylase sirtuin stimulators, NADPH oxidase (NOX) inhibitors, NFE2L2 gene inhibitors, nicotinic acid receptor 1 agonists, opioid receptor mu antagonists, P2Y13 purinoceptor stimulators, nuclear erythroid 2-related factor 2 stimulators, nuclear receptor modulators, P2X7 purinoceptor modulators, PACAP type I receptor agonists, PDE 3 inhibitors, PDE 4 inhibitors, PDE 5 inhibitors, PDGF receptor beta modulators, phenylalanine hydroxylase stimulators, phospholipase C inhibitors, phosphoric diester hydrolase inhibitors, PPAR alpha agonists, PPAR delta agonists, PPAR gamma agonists, peptidyl-prolyl cis-trans isomerase A inhibitors, PNPLA3 gene inhibitors, PPAR gamma modulators, protease-activated receptor-2 antagonists, protein kinase modulators, protein NOV homolog modulators, PTGS2 gene inhibitors, renin inhibitors, resistin/CAP1 (adenylyl cyclase associated protein 1) interaction inhibitors, rho associated protein kinase inhibitors, snitrosoglutathione reductase (GSNOR) enzyme inhibitors, sodium glucose transporter-2 inhibitors, sphingolipid delta 4 desaturase DES1 inhibitors, SREBP transcription factor inhibitors, STAT-1 inhibitors, stearoyl CoA desaturase-1 inhibitors, STK25 inhibitors, suppressor of cytokine signalling-1 stimulators, suppressor of cytokine signalling-3 stimulators, telomerase stimulators, TERT gene modulators, TGF beta (TGFB1) ligand inhibitors, TNF antagonists, transforming growth factor β (TGF-β), transforming growth factor β activated Kinase 1 (TAK1), thyroid hormone receptor beta agonists, TLR-4 antagonists, transglutaminase inhibitors, tyrosine kinase receptor modulators, GPCR modulators, nuclear hormone receptor modulators, TLR-9 antagonists, VDR agonists, WNT modulators, YAP/TAZ modulators and zonulin inhibitors.

Non-limiting examples of the one or more additional therapeutic agents include:
ACE inhibitors, such as enalapril;
Acetaldehyde dehydrogenase inhibitors, such as ADX-629;
Acetyl CoA carboxylase (ACC) inhibitors, such as NDI-010976 (firsocostat), DRM-01, gemcabene, GS-834356, PF-05175157, QLT-091382, PF-05221304;
Acetyl CoA carboxylase/Diacylglycerol O acyltransferase 2 inhibitors, such as PF-07055341;
Adenosine receptor agonists, such as CF-102 (namodenoson), CF-101, CF-502, CGS21680;
Adenosine A3 receptor antagonist, such as FM-101;
Adiponectin receptor agonists, such as ADP-355, ADP-399;
Adrenergic receptor antagonist, such as bromocriptine, phentermine, VI-0521
Aldehyde dehydrogenase 2 stimulators, such as FP-045;
Alpha glucosidase inhibitors (e.g. voglibose, acarbose, or miglitol);
Amylin/calcitonin receptor agonists, such as KBP-042, KBP-089;
AMP activated protein kinase stimulators, such as, C-455, PXL-770, O-304;
AMP kinase activators/ATP citrate lyase inhibitors, such as as bempedoic acid (ETC-1002, ESP-55016)
AMP activated protein kinase/Endothelial nitric oxide synthase/NAD-dependent deacetylase sirtuin-1 stimulators, such as NS-0200 (leucine+metformin+sildenafil); —Androgen receptor agonists, such as LPCN-1144, LPCN-1148, testosterone prodrug; —Angiotensin II AT-1 receptor antagonists, such as irbesartan; Angiopoietin-related protein-3 inhibitors, such as vupanorsen (IONIS-ANGPTL3-LRx);
Apelin receptor agonist, such as CB-5064, MBT-2;
Autophagy protein modulators, such as A-2906;
Autotaxin (ectonucleotide pyrophosphatase/phosphodiesterase 2 (NPP2 or ENPP2)) inhibitors, such as FP10.47, PAT-505, PAT-048, GLPG-1690, X-165, PF-8380, TJC-0265, TJC-0316, AM-063, BBT-877;
Axl tyrosine kinase receptor inhibitors, such as bemcentinib (BGB-324, R-428);
Bax protein stimulators, such as CBL-514;
Bioactive lipids, such as DS-102;
Cannabinoid receptor modulators, such as namacizumab (nimacimab), GWP-42004, REV-200, CRB-4001, INV-101, SCN-002;
Caspase inhibitors, such as emricasan;
Pan cathepsin B inhibitors, such as VBY-376;
Pan cathepsin inhibitors, such as VBY-825;
CCK receptor antagonist, such as proglumide;
CCL26 gene inhibitor, such as mosedipimod, KDDF-201410-10
CCR2/CCR5 chemokine antagonists, such as BMS-687681, cenicriviroc, maraviroc, CCX-872, leronlimab, WXSH-0213;
CCR2/CCR5 chemokine antagonists and FXR agonists, such as LJC-242 (tropifexor+cenivriviroc);
CCR2 chemokine antagonists, such as propagermanium;
CCR2 chemokine/Angiotensin II AT-1 receptor antagonists, such as DMX-200, DMX-250;
CCR3 chemokine antagonists, such as bertilimumab;
CD3 antagonists, such as NI-0401 (foralumab);
CDGSH iron sulfur domain protein modulators, such as EYP-002;
Chitinase inhibitor, such as OATD-01;
Chitotriosidase 1 inhibitors, such as OAT-2068;
Chloride channel stimulators, such as cobiprostone, and lubiprostone;
Casein kinase-1 (CK1) delta/epsilon inhibitors, such as PF-05006739;
Connective tissue growth factor ligand inhibitor, such as PBI-4050;
CXCR4 chemokine antagonists, such as AD-214;
Diglyceride acyltransferase 2 (DGAT2) inhibitors, such as IONIS-DGAT2Rx, PF-06865571;
Diglyceride acyltransferase 1 (DGAT1) inhibitors, such as GSK-3008356;
Diacylglycerol O acyltransferase 1 (DGAT1)/Cytochrome P4502E1 inhibitors (CYP2E1), such as SNP-610;
Dihydroorotate dehydrogenase inhibitor, such as vidofludimus;
Dipeptidyl peptidase IV inhibitors, such as linagliptin, evogliptin;
Eotaxin ligand inhibitors, such as bertilimumab, CM-101;
Extracellular matrix protein modulators, such as CNX-024;
Farnesoid X receptor (FXR) agonists, such as AGN-242266, AGN-242256, ASC-42, EDP-297 (EP-024297), RDX-023, BWL-200, AKN-083, EDP-305, GNF-5120, cilofexor tromethamine (GS-9674), HPG-1860, IOT-022, LMB-763, obeticholic acid, Px-102, Px-103, M790, M780, M450, M-480, MET-409, MET-642, PX20606, SYHA-1805, vonafexor (EYP-001), TERN-101, TC-100, INT-2228, TQA-3526, ZG-5266;
Farnesoid X receptor (FXR)/G-protein coupled bile acid receptor 1 (TGR5) agonists, such as INT-767;
Fatty acid synthase inhibitors, such as TVB-2640, FT-8225;
Fibroblast growth factor 19 (rhFGF19)/cytochrome P450 (CYP) 7A1 inhibitors, such as aldafermin (NGM-282);
Fibroblast growth factor 21 (FGF-21) ligand modulators, such as AP-025, BMS-986171, B-1654, BIO89-100, BOS-580,
Pegbelfermin (BMS-986036), B-1344;
Fibroblast growth factor 21 (FGF-21)/glucagon like peptide 1 (GLP-1) agonists, such as YH-25723 (YH-25724; YH-22241), efruxifermin (AKR-001);
FGF receptor agonists/Klotho beta stimulators, such as BFKB-8488A (RG-7992);
Free fatty acid receptor 1 agonist, such as SCO-267;
Galectin-3 inhibitors, such as belapectin (GR-MD-02), GB-1107 (Gal-300), GB-1211 (Gal-400);
GDNF family receptor alpha like agonist, such as NGM-395
Glucagon-like peptide 1 (GLP1R) agonists, such as ALT-801, AC-3174, liraglutide, cotadutide (MEDI-0382), SAR-425899, LY-3305677, HM-15211, YH-25723, YH-GLP1, RPC-8844, PB-718, PF-06882961, semaglutide;
Glucagon-like peptide 1 receptor agonist; Oxyntomodulin ligand; Glucagon receptor agonist, such as efinopegdutide;
Gastric inhibitory polypeptide/Glucagon-like peptide-1 (GIP/GLP-1) receptor co-agonist, such as tirzepatide (LY-3298176);
PEGylated long-acting glucagon-like peptide-1/glucagon (GLP-1R/GCGR) receptor dual agonist, such as DD-01;
Glucagon/GLP1-receptor agonist, such as BI-456906;
Glucocorticoid receptor antagonists, such as CORT-118335 (miricorilant);
Glucose 6-phosphate 1-dehydrogenase inhibitors, such as ST001;
Glucokinase stimulator, such as dorzagliatin, sinogliatin (RO-5305552)
G-protein coupled bile acid receptor 1 (TGR5) agonists, such as RDX-009, INT-777, HY-209;
G-protein coupled receptor 84 antagonist, such as PBI-4547;
G-protein coupled receptor-119 agonist, such as DA-1241;
Heat shock protein 47 (HSP47) inhibitors, such as ND-L02-s0201;
Hedgehog protein and/or TGF beta ligand inhibitors, such as Oxy-210
Histone deacetylase inhibitors/STAT-3 modulators, such as SFX-01;
HMG CoA reductase inhibitors, such as atorvastatin, fluvastatin, pitavastatin, pravastatin, rosuvastatin, simvastatin;
HSD17B13 gene inhibitor, such as ALN-HSD, ARO-HSD
Hydrolase inhibitor, such as ABD-X;
Hypoxia inducible factor-2 alpha inhibitors, such as PT-2567;
IL-10 agonists, such as peg-ilodecakin;
Ileal sodium bile acid cotransporter inhibitors, such as odevixibat (A-4250), volixibat potassium ethanolate hydrate (SHP-262), GSK2330672, CJ-14199, elobixibat (A-3309); —Insulin sensitizers, such as, KBP-042, azemiglitazone potassium (MSDC-0602K), ION-224, MSDC-5514, Px-102, RG-125 (AZD4076), Tolimidone, VVP-100X, CB-4211, ETI-101, pioglitazone;
Insulin ligand/dsInsulin receptor agonists, such as ORMD-0801;
Integrin antagonists, such as IDL-2965;
IL-6 receptor agonists, such as KM-2702;
Integrin alpha-V/beta-6 and alpha-V/beta-1 dual inhibitor; such as PLN-74809;
Interleukin 17 ligand inhibitor, such as netakimab
Jak1/2 tyrosine kinase inhibitor, such as baricitinib
Jun N terminal kinase-1 inhibitor, such as CC-90001
Kelch like ECH associated protein 1 modulator, such as alpha-cyclodextrin-stabilized sulforaphane;
Ketohexokinase (KHK) inhibitors, such as PF-06835919, LY-3478045;
beta Klotho (KLB)-FGF1c agonists, such as MK-3655 (NGM-313);
Leukotriene A4 hydrolase inhibitor, such as LYS-006;
5-Lipoxygenase inhibitors, such as tipelukast (MN-001), epeleuton (DS-102, (AF-102); —Lipoprotein lipase inhibitors, such as CAT-2003;
LPL gene stimulators, such as alipogene tiparvovec;
Liver X receptor (LXR) inhibitors, such as PX-665, PX-L603, PX-L493, BMS-852927, T-0901317, GW-3965, SR-9238;
Lysophosphatidate-1 receptor antagonists, such as BMT-053011, UD-009 (CP-2090), AR-479, ITMN-10534, BMS-986020, KI-16198;
Lysyl oxidase homolog 2 inhibitors, such as simtuzumab, PXS-5382A (PXS-5338); —Macrophage mannose receptor 1 modulators, such as tilmanocept-Cy3 (technetium Tc 99m tilmanocept);
Matrix metalloprotease inhibitors, such as ALS-L1023;
Membrane copper amine oxidase (VAP-1) inhibitors, such as TERN-201, TT-01025; —MEKK-5 protein kinase (ASK-1) inhibitors, such as CJ-16871, CS-17919, selonsertib (GS-4997), SRT-015, GS-444217, GST-HG-151, TERN-301;
MCH receptor-1 antagonists, such as CSTI-100 (ALB-127158);
Semicarbazide-Sensitive Amine Oxidase/Vascular Adhesion Protein-1 (SSAO/VAP-1) Inhibitors, such as PXS-4728A (BI-1467335);
Methionine aminopeptidase-2 inhibitors, such as ZGN-1061, ZGN-839, ZN-1345;
Methyl CpG binding protein 2 modulators, such as mercaptamine;
Mineralocorticoid receptor antagonists (MCRA), such as MT-3995 (apararenone);
Mitochondrial uncouplers, such as 2,4-dinitrophenol, HU6, Mito-99-0053;
Mixed lineage kinase-3 inhibitors, such as URMC-099-C;
Motile sperm domain protein 2 inhibitors, such as VB-601;
Myelin basic protein stimulators, such as olesoxime;
Myeloperoxidase inhibitors, such as PF-06667272, AZM-198;
NADPH oxidase inhibitors, such as GKT-831, GenKyoTex, APX-311, setanaxib;
Nicotinic acid receptor 1 agonists, such as ARI-3037MO;
NACHT LRR PYD domain protein 3 (NLRP3) inhibitors, such as KDDF-201406-03, NBC-6, IFM-514, JT-194 (JT-349);
NFE2L2 gene inhibitor, such as GeRP-amiR-144
Nuclear receptor modulators, such as DUR-928 (DV-928);
Opioid receptor mu antagonists, such as methylnaltrexone;
P2X7 purinoceptor modulators, such as SGM-1019;
P2Y13 purinoceptor stimulators, such as CER-209;
PDE 3/4 inhibitors, such as tipelukast (MN-001);
PDE 5 inhibitors, such as sildenafil, MSTM-102;
PDGF receptor beta modulators, such as BOT-191, BOT-509;
Peptidyl-prolyl cis-trans isomerase inhibitors, such as CRV-431 (CPI-432-32), NVP-018, NV-556 (NVP-025);
Phenylalanine hydroxylase stimulators, such as HepaStem;
Phosphoric diester hydrolase inhibitor, such as ZSP-1601;
PNPLA3 gene inhibitor, such as AZD-2693;
PPAR agonists, such as Chiglitazar, elafibranor (GFT-505), seladelpar lysine (MBX-8025), deuterated pioglitazone R-enantiomer, pioglitazone, PXL-065 (DRX-065), saroglitazar, lanifibranor (IVA-337), CHS-131, pemafibrate (K-877), ZG-0588, ZSP-0678; ZSYM-008;
Protease-activated receptor-2 antagonists, such as PZ-235;
Protein kinase modulators, such as CNX-014;
Protein NOV homolog modulators, such as BLR-200;
PTGS2 gene inhibitors, such as STP-705, STP-707;
Renin inhibitors, such as PRO-20;
Resistin/CAP1 (adenylyl cyclase associated protein 1) interaction inhibitors, such as DWJ-211
Rev protein modulator, such as ABX-464;
Rho associated protein kinase (ROCK) inhibitors, such as REDX-10178 (REDX-10325), KD-025, RXC-007, TDI-01;
Snitrosoglutathione reductase (GSNOR) enzyme inhibitors, such as SL-891;
Sodium glucose transporter-2 (SGLT2) inhibitors, such as ipragliflozin, remogliflozin etabonate, ertugliflozin, dapagliflozin, tofogliflozin, sotagliflozin,
Sodium glucose transporter-1/2 (SGLT 1/2) inhibitors, such as licogliflozin bis(prolinate) (LIK-066);
SREBP transcription factor inhibitors, such as CAT-2003, HPN-01, MDV-4463;
Stearoyl CoA desaturase-1 inhibitors, such as aramchol;
Thyroid hormone receptor beta agonists, such as ALG-009, ASC-41, CNPT-101101; CNPT-101207, CS-27186, KY-41111, resmetirom (MGL-3196), MGL-3745, TERN-501, VK-2809;
TLR-2/TLR-4 antagonists, such as VB-201 (CI-201);
TLR-4 antagonists, such as JKB-121, JKB-122, naltrexone;
Tyrosine kinase receptor modulators, such as CNX-025, GFE-2137 (repurposed nitazoxanide);
TLR-9 antagonist, such as GNKS-356
TNF antagonist, such as ALF-421
GPCR modulators, such as CNX-023;
Nuclear hormone receptor modulators, such as Px-102;
VDR agonist, such as CK-15;
Xanthine oxidase inhibitors, such as ACQT-1127;
Xanthine oxidase/Urate anion exchanger 1 (URAT1) inhibitors, such as RLBN-1001, RLBN-1127; and
Zonulin Inhibitors, such as lorazotide acetate (INN-202).

In some embodiments, the one or more additional therapeutic agents are selected from A-4250, AC-3174, acetylsalicylic acid, AK-20, alipogene tiparvovec, AMX-342, AN-3015, anti-TAGE antibody, aramchol, ARI-3037MO, ASP-8232, AZD-2693, bertilimumab, Betaine anhydrous, BI-1467335, BMS-986036, BMS-986171, BMT-053011, BOT-191, BTT-1023, budesonide, BX-003, CAT-2003, cenicriviroc, CBW-511, CER-209, CF-102, CGS21680, CNX-014, CNX-023, CNX-024, CNX-025, cobiprostone, colesevelam, dabigatran etexilate mesylate, dapagliflozin, DCR-LIV1, deuterated pioglitazone R-enantiomer, 2,4-dinitrophenol, DRX-065, DS-102, DUR-928, EDP-305, elafibranor (GFT-505), emricasan, enalapril, ertugliflozin, evogliptin, F-351, fluasterone (ST-002), FT-4101, GDD-3898, GH-509, GKT-831, GNF-5120, GRI-0621, GR-MD-02, GS-300, GS-4997, GS-9674, HEC-96719, HTD-1801, HS-10356, HSG-4112, HST-202, HST-201, HU-6, hydrochlorothiazide, icosabutate (PRC-4016), icosapent ethyl ester, IMN-124-E, INT-767, INV-240, IONIS-DGAT2Rx, ipragliflozin, Irbesarta, propagermanium, IVA-337, J2H-1702, JKB-121, KB-GE-001, KBLP-004, KBLP-009, KBP-042, KD-025, M790, M780, M450, metformin, sildenafil, LB-700, LC-280126, linagliptin, liraglutide, (LJN-452) (tropifexor), LM-011, LM-002 (CVI-LM-002), LMB-763, LYN-100, MB-N-008, MBX-8025, MDV-4463, mercaptamine, MGL-3196, MGL-3745, MP-301, MSDC-0602K, namacizumab, NC-101, NDI-010976, ND-L02-s0201 (BMS-986263), NGM-282, NGM-313, NGM-386, NGM-395, NP-011, NP-135, NP-160, norursodeoxycholic acid, NV-422, NVP-022, O-304, obeticholic acid (OCA), 25HC3S, olesoxime, PAT-505, PAT-048, PBI-4547, peg-ilodecakin, pioglitazone, pirfenidone, PRI-724, PX20606, Px-102, PX-L603, PX-L493, PXS-4728A, PZ-235, PZH-2109, RCYM-001, RDX-009, remogliflozin etabonate, RG-125 (AZD4076), RPI-500, S-723595, saroglitazar, SBP-301, semaglutide, SH-2442, SHC-028, SHC-023, simtuzumab, solithromycin, sotagliflozin, statins (atorvastatin, fluvastatin, pitavastatin, pravastatin, rosuvastatin, simvastatin), symbiotic, TCM-606F, TEV-45478, TQA-3526, TQA-3563, tipelukast (MN-001), TLY-012, TRX-318, TVB-2640, TXR-612, TS-20004, UD-009, UN-03, ursodeoxycholic acid, VBY-376, VBY-825, VK-2809, vismodegib, volixibat potassium ethanolate hydrate (SHP-626), VVP-100X, WAV-301, WNT-974, WXSH-0038, WXSH-0078, XEN-103, XRx-117, XTYW-003, XW-003, XW-004, ZGN-839, ZG-5216, ZSYM-008, ZYSM-007.

In some embodiments, the compound of present application is administered in combination with one or more therapeutic agents selected from the group consisting of anti-obesity agents, peptide YY and analogues thereof, neuropeptide Y receptor type 2 (NPYR2) agonists, NPYR1 agonists, NPYR5 antagonists, cannabinoid receptor type 1 (CB1 R) antagonists, lipase inhibitors (e.g., orlistat), human proislet peptides (HIP), melanocortin receptor 4 agonists (e.g., setmelanotide), melanin concentrating hormone receptor 1 antagonists, farnesoid X receptor (FXR) agonists (e.g. obeticholic acid), apoptotic signal-regulating kinase (ASK-1) inhibitors, zonisamide, phentermine (alone or in combination with topiramate), norepinephrine/dopamine reuptake inhibitors (e.g., buproprion), opioid receptor antagonists (e.g., naltrexone), combinations of norepinephrine/dopamine reuptake inhibitor and opioid receptor antagonists (e.g., a combination of bupropion and naltrexone), GDF-15 analogs, sibutramine, cholecystokinin agonists, amylin and analogues thereof (e.g., pramlintide), leptin and analogues thereof (e.g., metroleptin), serotonergic agents (e.g., lorcaserin), methionine aminopeptidase 2 (MetAP2) inhibitors (e.g., beloranib or ZGN-1061), phendimetrazine, diethylpropion, benzphetamine, SGLT2 inhibitors (e.g., empagliflozin, canagliflozin, dapagliflozin, ipragliflozin, tofogliflozin, sergliflozin etabonate, remogliflozin etabonate, or ertugliflozin), SGLTL1 inhibitors, dual SGLT2/SGLT1 inhibitors, fibroblast growth factor receptor (FGFR) modulators, AMP-activated protein kinase (AMPK) activators, biotin, MAS receptor modulators, glucagon receptor agonists (alone or in combination with another GLP-1 R agonist, e.g., liraglutide, exenatide, dulaglutide, albiglutide, lixisenatide, or semaglutide), insulin sensitizers such as thiazolidinediones (TZDs), peroxisome proliferator-activated receptor alpha (PPARα) agonists, fish oil, acetyl-coA carboxylase (ACC) inhibitors, transforming growth factor beta (TGFβ) antagonists, GDNF family receptor alpha like (GFRAL) agonists, a melanocortin-4 receptor (MC4R) agonists, including the pharmaceutically acceptable salts of the specifically named agents and the pharmaceutically acceptable solvates of said agents and salts.

### EXAMPLES

### Synthesis

The compounds of the disclosure may be prepared using methods disclosed herein and routine modifications thereof which will be apparent given the disclosure herein and methods well known in the art. Conventional and well-known synthetic methods may be used in addition to the teachings herein. The synthesis of typical compounds of Formula I-A-1, or a pharmaceutically acceptable salt thereof, e.g., compounds having structures described by one or more of Formula I-A-1, or other formulas or compounds disclosed herein, may be accomplished as described in the following examples.

### General Syntheses

Typical embodiments of compounds in accordance with the present application may be synthesized using the general reaction schemes and/or examples described below. It will be apparent given the description herein that the general schemes may be altered by substitution of the starting materials with other materials having similar structures to result in products that are correspondingly different. Descriptions of syntheses follow to provide numerous examples of how the starting materials may vary to provide corresponding products. Starting materials are typically obtained from commercial sources or synthesized using published methods for synthesizing compounds which are embodiments of the present application, inspection of the structure of the compound to be synthesized will provide the identity of each substituent group The identity of the final product will generally render apparent the identity of the necessary starting materials by a simple process of inspection, given the examples herein. Group labels (e.g., R1, R2) used in the reaction schemes herein are for illustrative purposes only and unless otherwise specified do not necessarily match by name or function the labels used elsewhere to describe compounds of Formula I-A-1 or aspects or fragments thereof.

### Synthetic Reaction Parameters

The compounds of this disclosure can be prepared from readily available starting materials using, for example, the following general methods and procedures. It will be appreciated that where typical or preferred process conditions (i.e., reaction temperatures, times, mole ratios of reactants, solvents, pressures, etc.) are given; other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts (1999) Protecting Groups in Organic Synthesis, 3rd Edition, Wiley, New York, and references cited therein.

Furthermore, the compounds of the present application may contain one or more chiral centers. Accordingly, if desired, such compounds can be prepared or isolated as pure stereoisomers, i.e., as individual enantiomers or diastereomers or as stereoisomer-enriched mixtures. All such stereoisomers (and enriched mixtures) are included within the scope of this disclosure, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents, and the like.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA). Others may be prepared by procedures or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplemental (Elsevier Science Publishers, 1989) organic Reactions, Volumes 1-40 (John Wiley, and Sons, 1991), March's Advanced Organic Chemistry, (John Wiley, and Sons, 5th Edition, 2001), and Larock's Comprehensive Organic Transformations (VCH Publishers Inc., 1989).

The terms “solvent,” “inert organic solvent” or “inert solvent” refer to a solvent inert under the conditions of the reaction being described in conjunction therewith (including, for example, benzene, toluene, acetonitrile, tetrahydrofuran (“THF”), N, N-dimethylformamide (“DMF”), chloroform, methylene chloride (or dichloromethane), diethyl ether, methanol, pyridine and the like). Unless specified to the contrary, the solvents used in the reactions of the present application are inert organic solvents, and the reactions are carried out under an inert gas, preferably nitrogen.

The term “q.s.” means adding a quantity sufficient to achieve a stated function, e.g., to bring a solution to the desired volume (i.e., 100%).

Compounds as provided herein may be synthesized according to the general schemes provided below. In the Schemes below, it should be appreciated that each of the compounds shown therein may have protecting groups as required present at any step. Standard protecting groups are well within the pervue of one skilled in the art.

### Example 1: Synthesis of the Compound of Formula I-A-1

In another aspect, the present invention provides a method for preparing a compound of Formula I-A-1, a pharmaceutically acceptable salt, an ester or a stereoisomer thereof, the method comprising six general routes (Route 1, Route 2, Route 3, Route 4, Route 5 and Route 6):

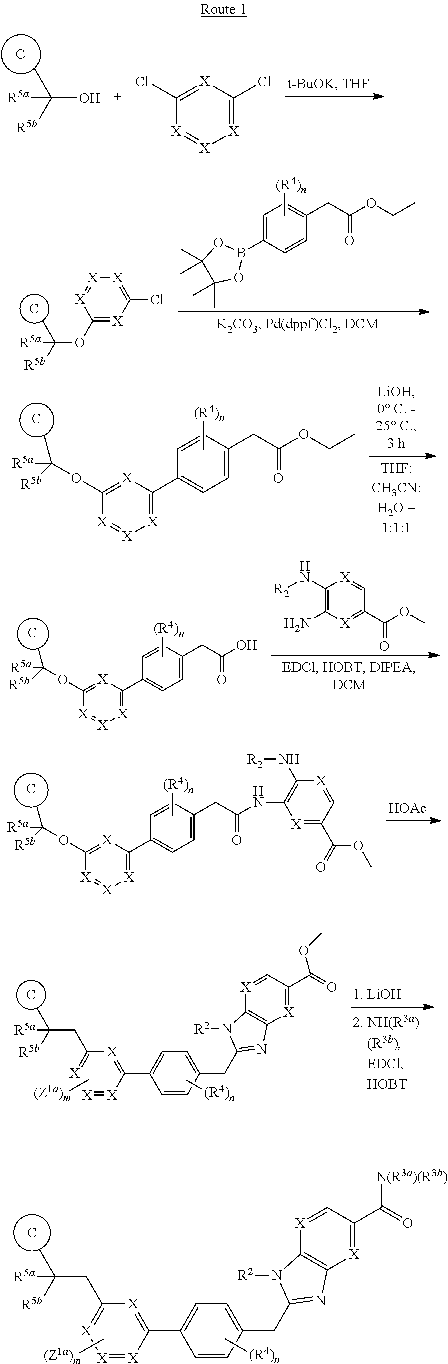

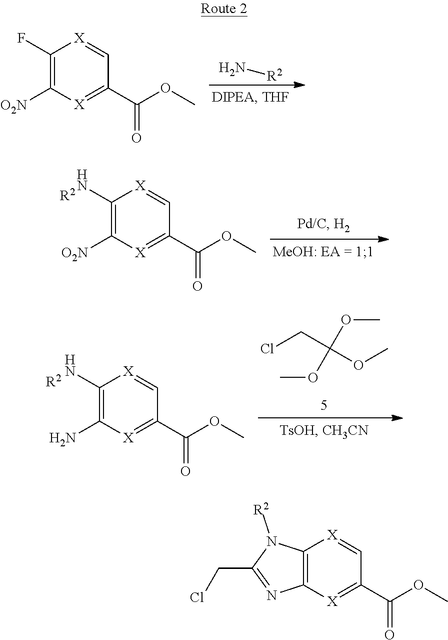

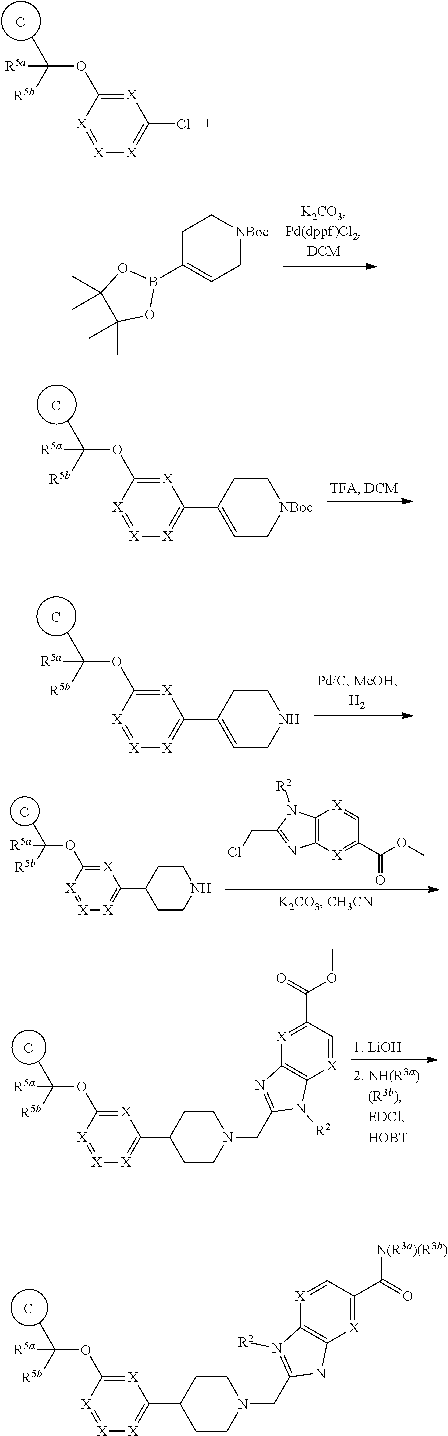

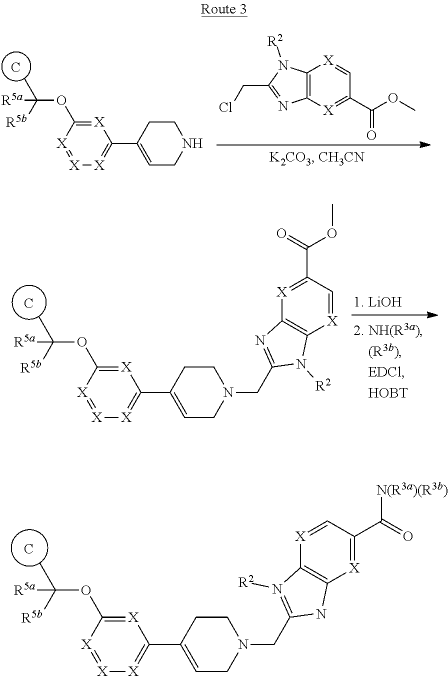

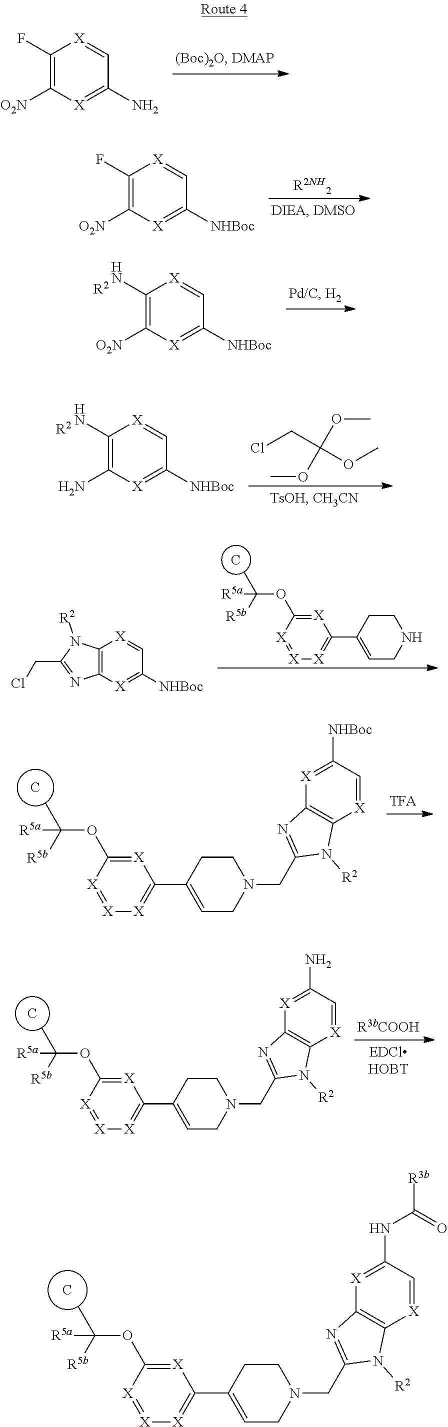

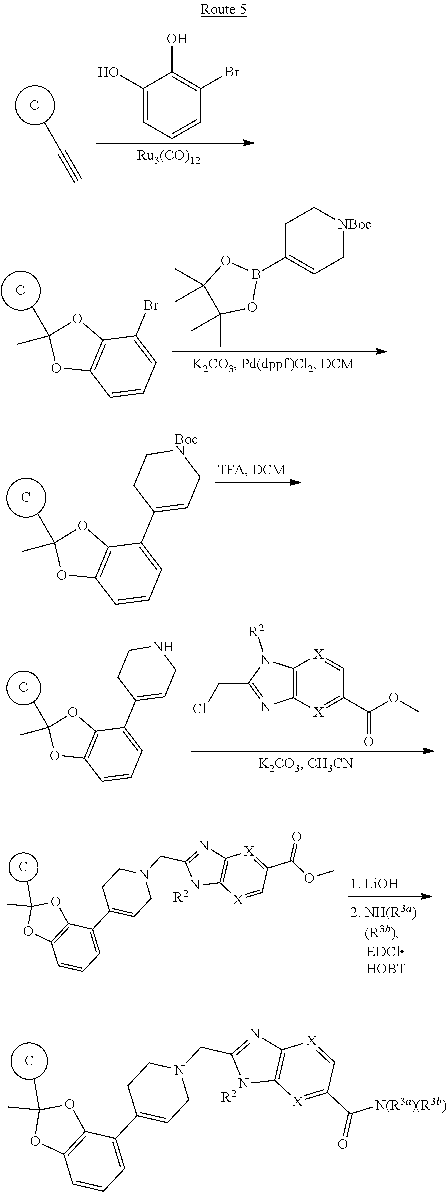

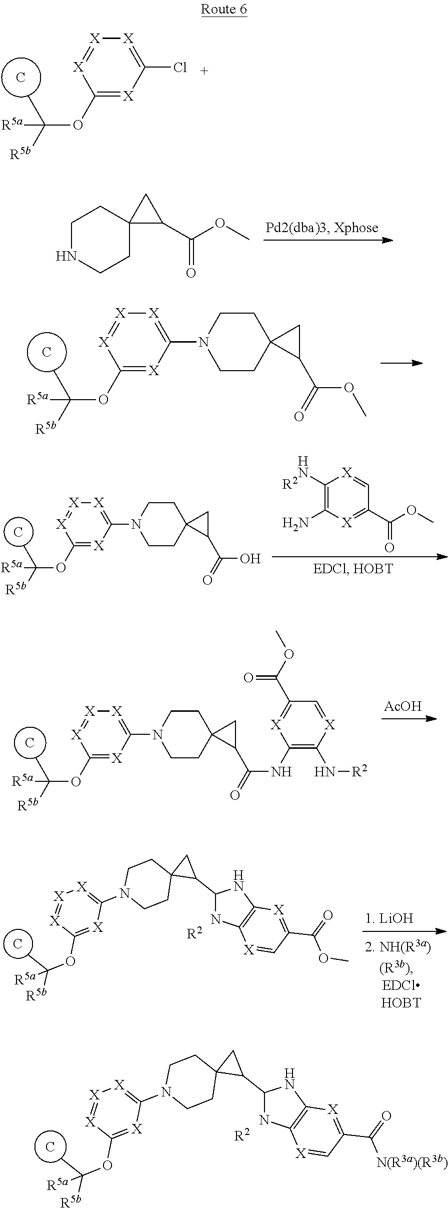

Preparation of Intermediate

The intermediate can be synthesized by follow 5 routes:

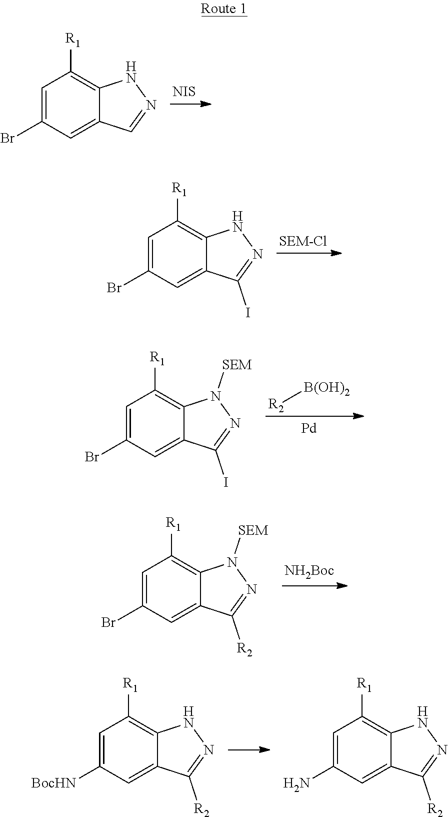

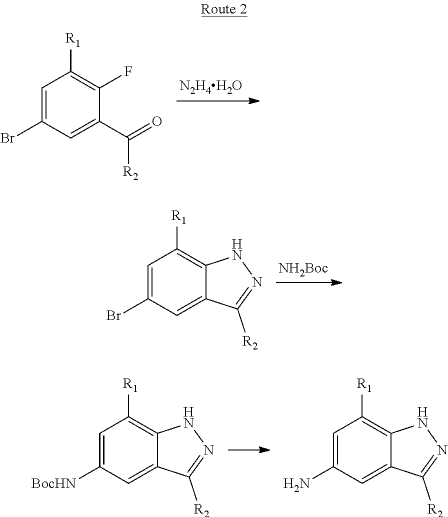

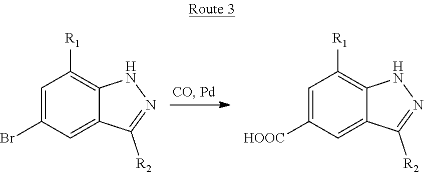

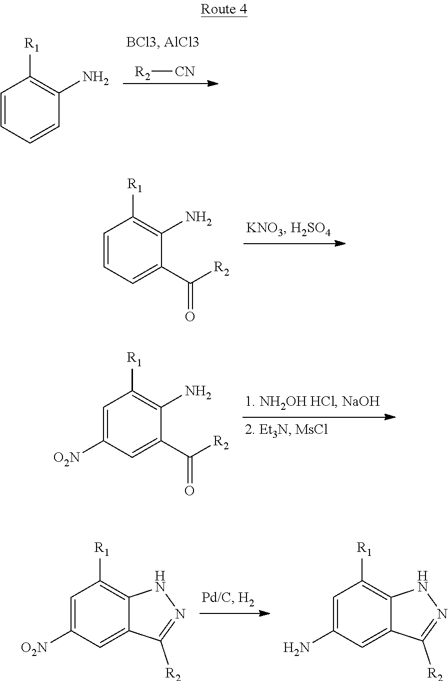

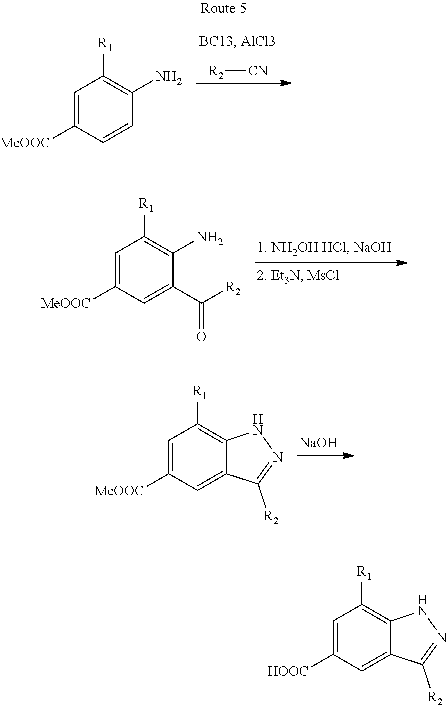

### The Preparation of 3,7-dimethyl-1H-indazol-5-amine (Int A)

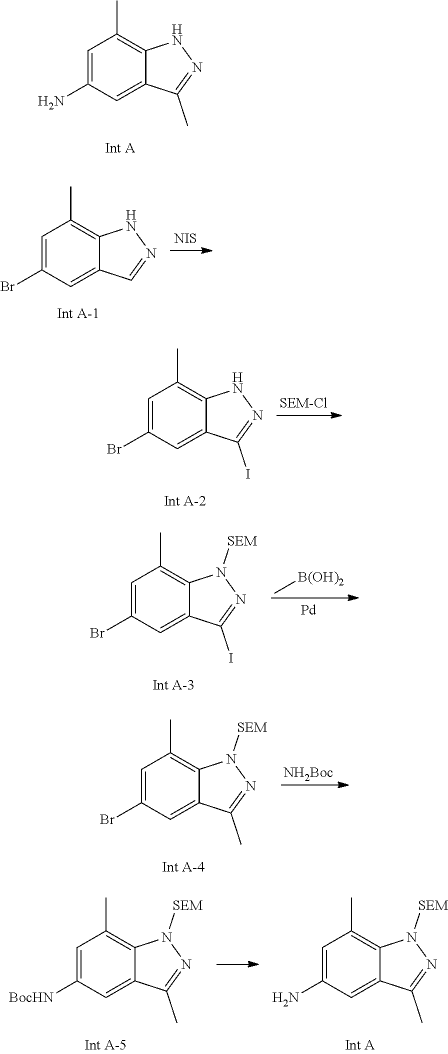

NIS (N-Iodosuccinimide) (29.0 g, 1.1 eq.) was added to a solution of 5-bromo-7-methyl-1H-indazole (25.0 g) in DCE (1,2-dichloroethane) (200 ml) at room temperature (RT) and the mixture was stirred at 80° C. for 12 h. After cooling to room temperature, the reaction mixture was diluted with THF (tetrahydrofuran), washed with Na₂S₂SO₃ (aq.) and brine, dried over anhydrous Na₂SO₄ and concentrated to obtain the desired product Int A-2 as yellow solid which was further purified by washing with PE (petroleum ether) (35.0 g, yield: 87.7%).

NaH (11.5 g, 2.5 eq.) was added to a solution of Int A-2 (40 g) and SemCl (40 g, 2.1 eq.) in THF (250 ml) with stirring at 0° C. The reaction mixture was stirred for 2 h at 25° C. The reaction mixture was quenched with H₂O, and extracted with EA (ethyl acetate), the organic phase was concentrated in vacuo to obtain Int A-3, 45.4 g, yield: 81.9%.

Pd₂(dppf)Cl₂ (0.9 g), K₃PO₄ (11.04 g, 2.7 eq.), and CH₃B(OH)₂ (1.96 g, 1.7 eq.) were added to a solution of Int A-3 (9.0 g) in dioxane with stirring at 25° C. The mixture was stirred for 3 h at 95° C. After cooling the mixture, Filter out the solid, then the filtrate was concentrated in vacuo to obtain Int A-4, the residue was purified by silica gel column chromatogram (EA/PE) to obtained the title compound as colored solid, 5.4 g, yield: 78.9%.

NH₂Boc (5.2 g, 4 eq.), Cs₂CO₃ (14.4 g, 4 eq.), Pd₂(dba)₃ (1.2 g, 0.15 eq) and X-phos (1.8 g, 0.3 eq) were added to a solution of Int A-4 (4.0 g) in Dixoane (100 ml) at RT and the mixture was stirred at 100° C. for 4 h under N₂. After cooling the reaction, the mixture was quenched with water (100 ml) and extracted with EA (100 ml×2). The residue was purified by silica gel column chromatogram (EA/PE=1/40-1/10) to obtain Int A-5, 3.6 g, yield: 81.7%.

HCl/EA (2M, 10 ml) was added to a solution of Int A-5 (3.6 g) in MeOH (5 ml) was added the mixture was stirred for 4 h at 40° C. A white solid was precipitated, and filtrate to obtain Int A, 1.4 g, yield: 94.5%. ¹H NMR (500 MHz, Chloroform-d) δ 11.40 (s, 1H), 6.85 (d, J=2.2 Hz, 1H), 6.68 (d, J=2.0 Hz, 1H), 4.35 (d, J=5.1 Hz, 1H), 4.33 (d, J=5.1 Hz, 1H), 2.95 (s, 3H), 2.65 (s, 3H).

### The Preparation of 7-fluoro-3-methyl-1H-indazol-5-amine (Int B)

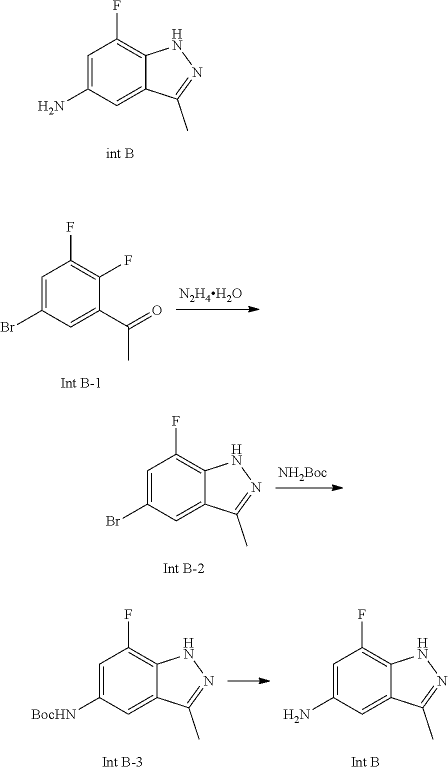

Int B-1 (5.0 g) and hydrazinium hydroxide solution (98%, 2.7 g, 2.5 eq) were added into NMP (N-methylpyrrolidone) (15 ml). The mixture was stirred at 150° C. for 4 h under N₂. After cooling the reaction, a white solid was precipitated and filtrated to obtain Int B-2, 3.8 g, 78.0%.

The next series of reactions can follow the Preparation of Int A, 7-fluoro-3-methyl-1H-indazol-5-amine was obtained.

### The Preparation of 3,7-dimethyl-1H-indazole-5-carboxylic acid (Int C)

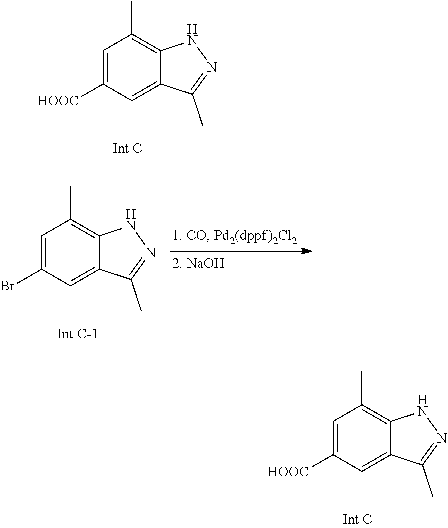

Int C-1 (2.0 g), trimethylamine (2.24 g, 2.5 eq.), 2,2′-bis(diphenylphosphino)-1,10-binaphthyl (0.55 g, 0.1 eq.) and PdCl₂ (0.31 g, 0.2 eq.) was added into methanol (20 ml) and acetonitrile (20 ml). The mixture was placed under an atmosphere of carbon monoxide (40 bar), stirred vigorously at 100° C. for 18 hours, cooled and exposed to air. The cooled reaction mixture was then filtered through Celite, the solvent was removed in vacuo. NaOH (4M, 5 ml) was added to the filtrate and stirred 30 min. HCl (2M) was then added to the mixture until pH 6 is reached. A white solid was precipitated and filtrated to obtain Int C, 0.7 g, yield: 41.4%. ¹H NMR (500 MHz, Chloroform-d) δ 8.44 (d, J=2.2 Hz, 1H), 7.84 (d, J=2.0 Hz, 1H), 2.97 (s, 3H), 2.66 (s, 3H).

### The Preparation of 3-cyclopropyl-7-methyl-1H-indazol-5-amine (Int D)

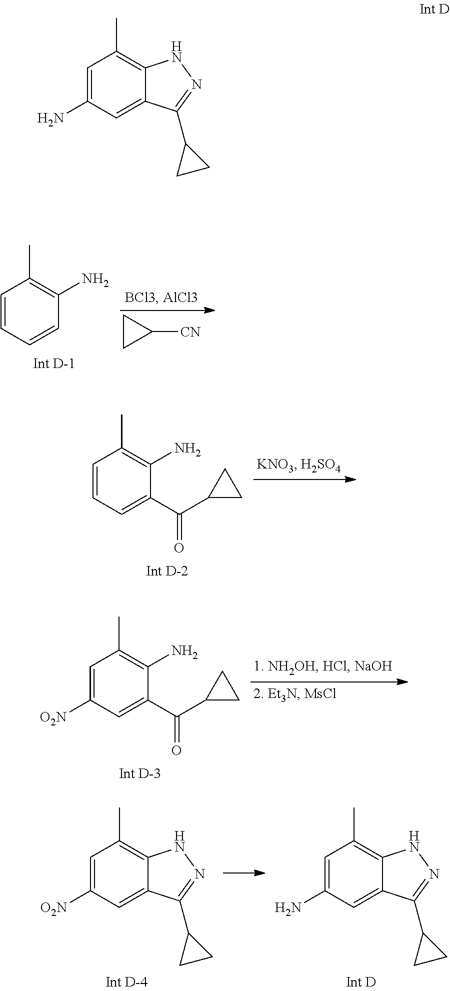

A solution of 2-isopropylaniline (Int D-1, 2.14 g, 0.02 mol) in 1,2-dichloroethane (10 ml) was added to a solution of boron trichloride (20 ml, 0.02 mol, 1M in toluene) in dichloromethane with stirring at 0° C. Aluminum chloride (2.96 g, 0.022 mol) and cyclopropanecarbonitrile (2.01 g, 0.03 mol) were then added to the mixture. The reaction mixture was allowed to reflux at 80° C. for 20 h. The reaction mixture was then cooled to 0° C., followed by addition of 2N HCl (10 ml). The reaction mixture was heated to 80° C. and stirred for an additional 30 min. The reaction mixture was then cooled to room temperature, extracted with dichloromethane. The organic layer was washed with 1N NaOH (10 ml) and brine, dried over anhydrous MgSO₄. The solvent was removed under reduced pressure. The residue was purified by flash column chromatography to produce the title compound Int D-2 (2.1 g, 60%) as a yellow solid.

KNO₃ (1.38 g, 13.7 mmol) was added slowly into a solution of Int D-2 (2.0 g, 11.4 mmol) in H₂SO₄ (98%, 8 ml) with stirring at 0° C., and stirred for 2 hours. The mixture solution was slowly added into the ice water (50 ml), and ethyl acetate (50 ml) was added to extract to generate an organic layer. The organic layer was removed under reduced pressure to obtain Int D-3 (1.9 g, 76%) as a yellow solid.

Sodium hydroxide (2.7 g, 68.8 mmol) was added to a solution of Int D-3 (1.9 g, 8.6 mmol) and hydroxylamine hydrochloride (1.8 g, 25.8 mmol) in water (2 ml) and ethanol (8 ml) with stirring under nitrogen at 0° C. Subsequent to the addition, the mixture was refluxed at 80° C. for 1 h. The mixture was concentrated, and the residue was dissolved in water (14 ml) and extracted with ethyl acetate (20 ml×2) to get an organic layer and concentrated in vacuo. The residue was dissolved in dichloromethane (10 ml). Triethylamine (1.74 g, 17.2 mmol) was added slowly, and the reaction mixture was stirred for 15 min at ambient temperature before being cooled to 0-5° C. in an ice/water bath. Methanesulfonyl chloride (1.25 g, 10.9 mmol) was added to 30 mL dichloromethane, and the resulting solution was cooled to 0-5° C. The cold solution of methanesulfonyl chloride was added in dropwise to the mixture. The resulting solution was stirred at 0-5° C. for 1.5 h. Silica gel was added, and the green/brown solution was concentrated by rotary evaporation. The residue was purified by flash column chromatography to produce the title compound Int D-4 (1.65 g, 88%) as a yellow solid.

Int D-4 (1.65 g, 7.6 mmol) and Pd/C (0.16 g, 10% w/w, 30% water) was added into methanol (10 ml) on a 50 ml single-necked round-bottomed flask. A hydrogen balloon was load on the flask, and stirred 4 hours until the yellow was disappeared. After filtration, the filtrate was concentrated in vacuo to obtain Int D (1.25 g, 88%) as a brown oil. LCMS (ESI): calculated for C11H13N3; [M+H]⁺: 188.1, found: 188.1. ¹H NMR (500 MHz, Chloroform-d) δ 7.15 (d, J=2.2 Hz, 1H), 6.84 (d, J=2.2 Hz, 1H), 4.42-4.32 (m, 2H), 2.66 (s, 3H), 2.24-2.19 (m, 1H), 1.03-0.87 (m, 4H).

### The Preparation of 7-fluoro-3-methyl-1H-indazole-5-carboxylic acid (Int E)

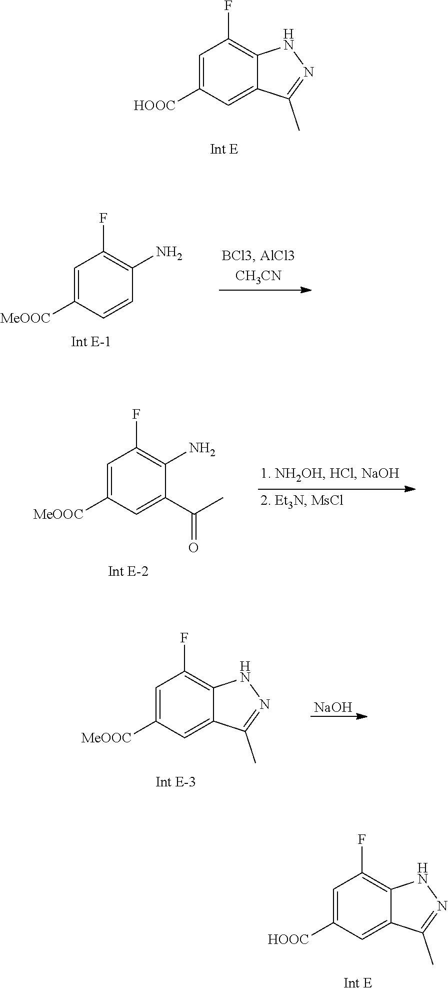

A solution of methyl 4-amino-3-fluorobenzoate (Int E-1, 3.38 g, 0.20 mol) in 1, 2-dichloroethane (10 mL) was added to a solution of boron trichloride (20 ml, 20 mmol, 1M in toluene) in dichloromethane with stirring at 0° C. To the mixture was added aluminum chloride (2.94 g, 22 mol) and acetonitrile (1.2 g, 30 mmol). The reaction mixture was allowed to reflux at 80° C. for 20 h. After cooling, 2N HCl (10 ml) was added. Then reaction mixture was heated to 80° C. and stirred for an additional 30 min. The reaction mixture was then cooled to room temperature, and extracted with dichloromethane. The organic layer was washed with 1N NaOH (10 ml) and brine, dried over anhydrous MgSO₄. The solvent was removed under reduced pressure. The residue was purified by flash column chromatography to give the title compound (Int E-2)(2.98 g, 70.65%) as a yellow solid.

Sodium hydroxide (2.3 g, 57.6 mmol) was added to solution of Int E-2 (2 g, 9.5 mmol) and hydroxylamine hydrochloride (1.97 g, 28.5 mmol) in water (2 ml) and ethanol (8 mL) with stirring under nitrogen at 0° C. Subsequent to the addition, the mixture was refluxed at 80° C. for 1 h. The mixture was concentrated, and the residue was dissolved in water (14 ml) and extracted with ethyl acetate (20 ml×2) to generate an organic layer and concentrated in vacuo. The residue was dissolved in dichloromethane (10 ml). Triethylamine (1.6 g, 14.8 mmol) was added slowly, and the reaction mixture was stirred for 15 min at ambient temperature before being cooled to 0-5° C. in an ice/water bath. Methanesulfonyl chloride (1.08 g, 9.5 mmol) was added to 30 ml dichloromethane, and the resulting solution was cooled to 0-5° C. The cold solution of methanesulfonyl chloride was added in dropwise to the mixture. The resulting solution was stirred at 0-5° C. for 1.5 h. Silica gel was added, and the green/brown solution was concentrated by rotary evaporation. The residue was purified by flash column chromatography to give the title compound Int E-3 (1.43 g, 73%) as a yellow solid.

Int E-3 (1.43 g, 6.87 mmol) and NaOH (1.1 g, 27.5 mmol) was added into methanol (10 ml) and water (5 ml), and stirred 1 hour at 60° C. After cooling, the mixture reaction was concentrated in vacuo. The reside was dissolved into water (10 ml), and 1N HCl was added to adjust the pH to 7. Then ethyl acetate (20 ml×2) was added to extract to obtain an organic layer. The solvent was removed under reduced pressure to obtain the Int E (1.25 g, 94%) as a white solid. LCMS (ESI): calculated for C9H7FN2O2; [M+H]⁺: 195.0, found: 195.0. ¹H NMR (500 MHz, Chloroform-d) δ 11.22 (s, 1H), 8.36 (d, J=2.2 Hz, 1H), 7.705 (dd, J=7.9, 2.0 Hz, 1H), 2.95 (s, 3H).

### The Preparation of tert-butyl (2-(chloromethyl)-1-((1-ethyl-1H-imidazol-5-yl)methyl)-1H-benzo[d]imidazol-5-yl)carbamate (Int F)

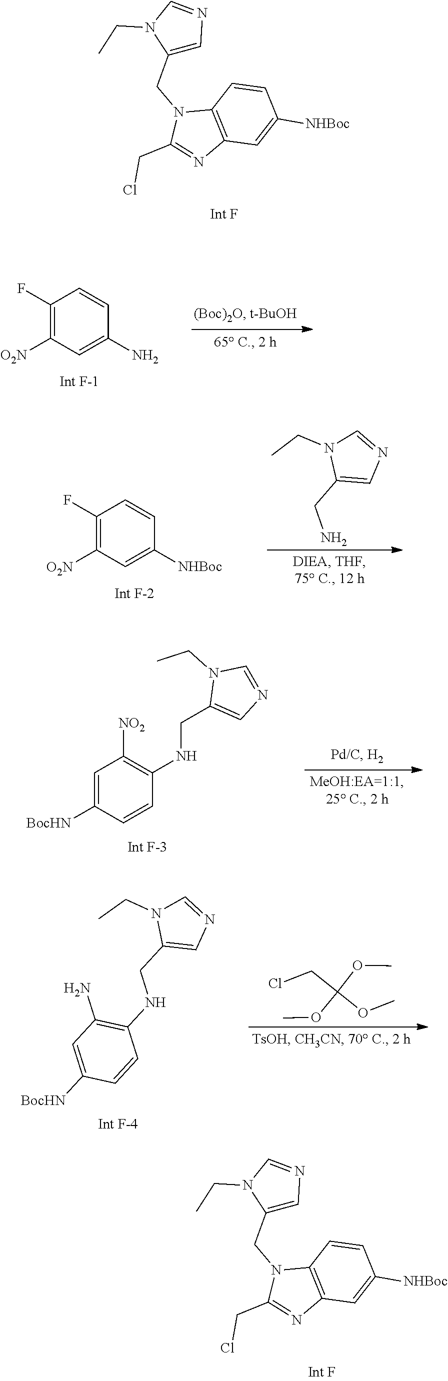

Di-tert-butyl dicarbonate (30.88 g, 141.66 mmol) was added portion-wise to a solution of Int F-1 (17.0 g, 108.97 mmol) in tert-butanol (250 ml). The reaction mixture was stirred at 55° C. for 8 hours. The resulting mixture was concentrated in vacuo. Trituration of the residue at 0° C. with petroleum ether (300 ml) provided Int F-2 as a pale white solid (26 g 93.2%). LCMS m/z 257.2 [M+H]+.

1-(1-Ethyl-1H-imidazol-5-yl)methanamine dihydrochloride (2.18 g, 11 mmol) and K₂CO₃ (6.95 g, 50 mmol) were added to a solution of Intermediate Int F-2 (2.56 g, 10 mmol) in DMF (N,N-dimethylformamide) (30 ml). The reaction mixture was stirred at 70° C. for 16 h. The reaction mixture was poured into water (300 ml) and then extracted with EtOAc (400 ml×2). The combined organic extracts were washed with brine (200 ml×2), dried over Na₂SO₄, filtered and concentrated under reduced pressure. The crude product was purified by flash chromatography (0 to 5% MeOH/DCM (dichloromethane)) to give Int F-3 (2.2 g, 61%) as a pale red solid. LCMS m/z 362.40 [M+H]+.

Int F-3 (181 mg, 0.501 mmol) was suspended in MeOH (150 ml) and treated with 10% palladium on carbon (20 mg) and the mixture was stirred at room temperature under 50 psi H₂ for 1 hours. The mixture was filtered through Celite® and the filtrate was concentrated under reduced pressure to yield Int F-4 (154 mg, 93%) as a pale brown solid which was used directly in the next step. LCMS m/z 332.20 [M+H+].

2-chloro-1,1,1-trimethoxy ethane (143 mg, 0.93 mmol) and pyridin-1-ium 4-methylbenzenesulfonate (23.2 mg, 0.093 mmol) were added to a solution of Int F-4 (123 mg, 0.37 mmol) in MeCN (5 ml). The mixture was heated to 70° C. for 1 h. The reaction was cooled to RT and concentrated under reduced pressure. The resultant crude product was triturated in 50% EtOAc/heptane. The solids were collected by filtration to obtain Int F (102 mg, 70%) as a brown solid. LCMS m/z 390.10 [M+H+].

### The Preparation of 2-bromo-6-((4-chloro-2-fluorobenzyl)oxy)pyridine (Int G)

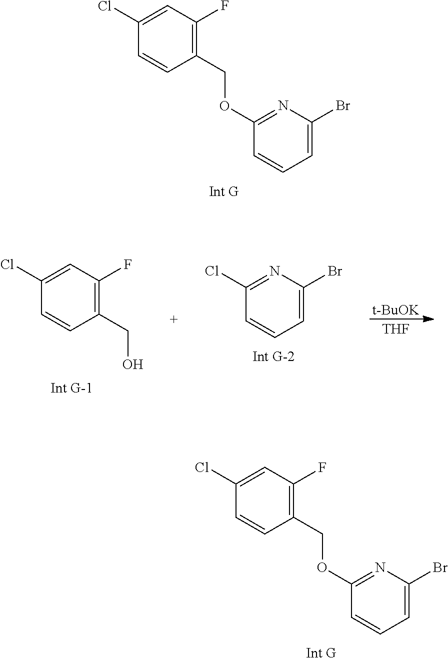 [SMILES: "t-BuOK" ; Fc1cc(Cl)ccc1COc1cccc(Br)n1 ; OCc1ccc(Cl)cc1F ; Clc1cccc(Br)n1 ; Fc1cc(Cl)ccc1COc1cccc(Br)n1 ; "THF"]

t-BuOK (21.0 g, 187 mmol) was added to a solution of Intermediate Int G-1 (20.0 g, 124.6 mmol) in THF (150 ml). The reaction mixture was stirred at 10˜15° C. for 45 min. Then Int G-2 (20 g, 104 mmol) was added into the mixture, and stirred overnight at room temperature. H₂O (500 ml) was added and filtered to obtain the Int G (22.0 g, yield: 79.7%).

### The Preparation of tert-butyl (2-(5-fluoro-2-(hydroxymethyl)-4-(4,4,5-trimethyl-1,3,2-dioxaborolan-2-yl)benzyl)-1-(((S)-oxetan-2-yl)methyl)-1H-benzo[d]imidazol-5-yl)carbamate (Int H)

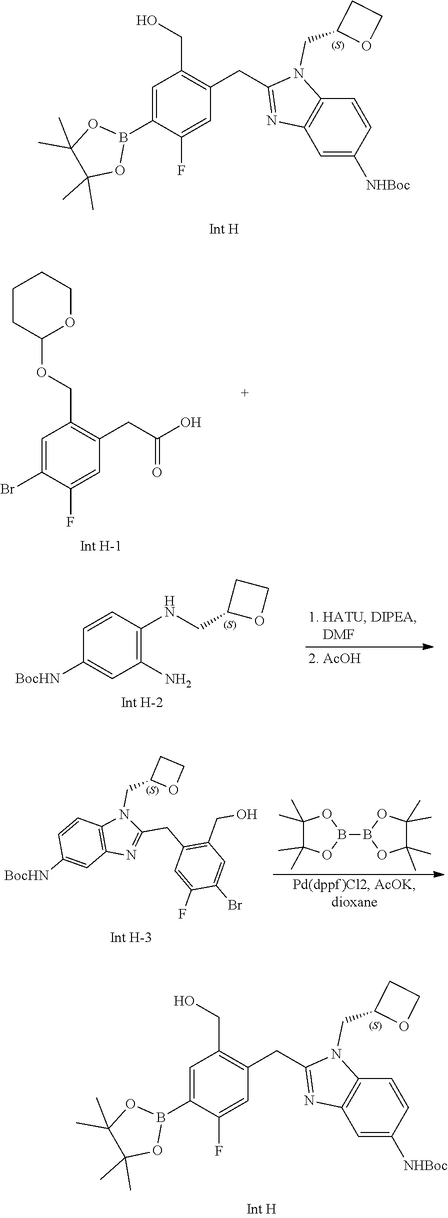

Int H-1 (1.0 g, 2.88 mmol), Int H-2 (0.93 g, 3.17 mmol), and HATU (1.44 g, 3.45 mmol) were taken up in N,N-dimethylformamide (10 ml) and N,Ndiisopropylethylamine (2.5 ml, 14.4 mmol) was added. The mixture was stirred at RT for one hour then diluted with saturated aqueous NH₄Cl (10 ml) and extracted with EtOAc (10 ml×3). The combined organics were dried over MgSO₄ and concentrated in vacuo. The resulting residue was taken up in acetic acid (25 ml) and the mixture heated to 100° C. for one hour. Following this time, the mixture was cooled to RT, concentrated in vacuo, and the material purified by normal phase column chromatography (eluent: EtOAc/PE) to yield the product Int H-3 (1.2 g, yield: 66.9%).

Under N₂, Int H-3 (1.0 g, 1.92 mmol), Bis(pinacolato)diboron (512.4 mg, 2.02 mmol), AcOK (282.3 mg, 2.88 mmol), Pd(dppf)Cl₂ (139 mg, 0.2 mmol) were taken into the dioxane (10 ml), and stirred 2 hours at 90˜95° C. After cooling, water (10 ml) was added and extracted with EtOAc (10 ml×3). The combined organics were dried over MgSO₄ and concentrated in vacuo. The resulting residue was purified by crystallization with EtOH (3 ml) to obtain Int H (0.85 g, yield: 78%). LCMS (ESI): calculated for C₃₀H₃₉BFN₃O₆; [M+H]⁺: 568.3, found: 568.3.

### The Preparation of tert-butyl (S)-(2-(chloromethyl)-1-(oxetan-2-ylmethyl)-1H-benzo[d]imidazol-5-yl)carbamate (Int I)

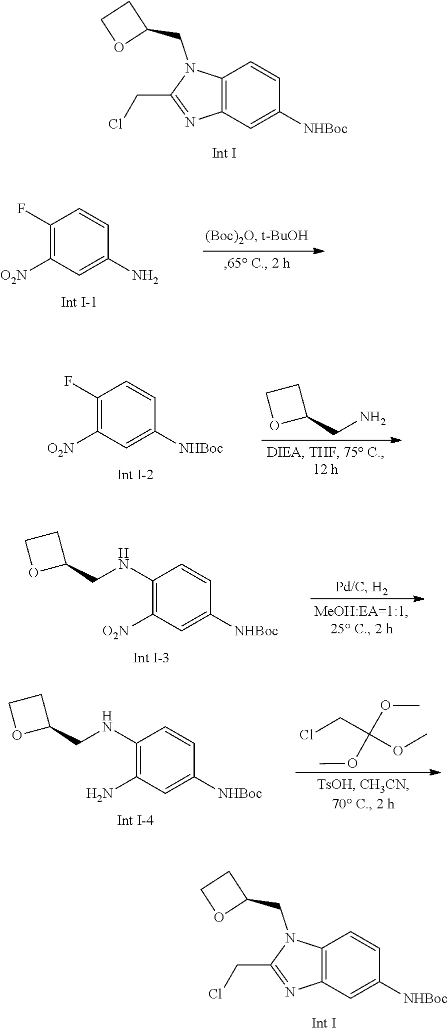

(Boc)₂O (42.0 g, 192 mmol) was added to a solution of 4-fluoro-3-nitroaniline (15.0 g, 96.0 mmol) in t-BuOH (150 ml) with stirring at 25° C. The reaction mixture was stirred at 40° C. for 12 h. The mixture was concentrated in vacuo, The crude product was slurried by PE to obtain tert-butyl (4-fluoro-3-nitrophenyl)carbamate (21.6 g, 88% yield) as a yellow solid. LCMS (ESI): calculated for C₁₁H₁₃FN₂O₄; [M+H]⁺: 256.2, found: 256.2.

N-ethyl-N-isopropyl-propan-2-amine (26.5 g, 205.1 mmol) was added to a solution of [(2S)-oxetan-2-yl]methanamine (9.3 g, 106.5 mmol) and compound 2 (21.0 g, 81.9 mmol) in THF (150 ml) with stirring at 25° C. The reaction mixture was stirred at 60° C. for 12 h. The mixture was diluted with EtOAc (100 ml) and H₂O (200 ml). The layers were separated, and the aqueous layer was extracted with EtOAc (100 ml×2). The combined organic phase was washed with brine (200 ml) and dried over anhydrous Na₂SO₄, filtered and concentrated in vacuo. The crude product was slurried by methyl tert-butyl ether to produce tert-butyl (S)-(3-nitro-4-((oxetan-2-ylmethyl)amino)phenyl)carbamate (23.6 g, 89.1% yield) as a yellow solid. LCMS (ESI): calculated for C₁₅H₂₁N₃O₅; [M+H]⁺: 323.3, found: 323.3.

10% Pd/C (4.6 g) was added to a solution of compound Int I-3 (23.0 g, 71.1 mmol) in EtOAc:MeOH=1:1 (200 ml) with stirring at 25° C. The reaction mixture was stirred at 25° C. for 12 h under hydrogen. The mixture was filtered and the filtrate cake was washed with EtOAc (100 ml×2). The combined organic phase concentrated in vacuo to obtain tert-butyl (S)-(3-amino-4-((oxetan-2-ylmethyl)amino)phenyl)carbamate (18.8 g, 90% yield) as a brown solid.

LCMS (ESI): calculated for C₁₅H₂₃N₃O₃; [M+H]⁺: 293.4, found: 293.4.

2-chloro-1,1,1-trimethoxyethane (11.4 g, 73.6 mmol) and pyridinium p-Toluenesulfonate (1.23 g, 4.9 mmol) were added to a solution of tert-butyl (S)-(3-amino-4-((oxetan-2-ylmethyl)amino)phenyl)carbamate (18.0 g, 61.4 mmol) in CH₃CN (150 ml) with stirring at RT. The reaction mixture was stirred at 70° C. for 2 h and upon completion of the reaction. The mixture was added EtOAc (100 ml) and H₂O (200 ml). The organic layers were separated, and the aqueous layer was extracted with EtOAc (100 ml×2). The combined organic phase was washed with brine (200 ml) and dried over anhydrous Na₂SO₄, filtered and concentrated in vacuo. The crude product was purified by flash column chromatography (petroleum ether/ethyl acetate 3:1) to produce Int I: tert-butyl (S)-(2-(chloromethyl)-1-(oxetan-2-ylmethyl)-1H-benzo[d]imidazol-5-yl)carbamate (18.0 g, 83% yield) as a yellow solid. LCMS (ESI): calculated for C₁₇H₂₂ClN₃O₃; [M+H]⁺: 352.1, found: 352.1.

### The Preparation of methyl (S)-2-(chloromethyl)-1-(oxetan-2-ylmethyl)-1H-benzo[d]imidazole-5-carboxylate (Int J)

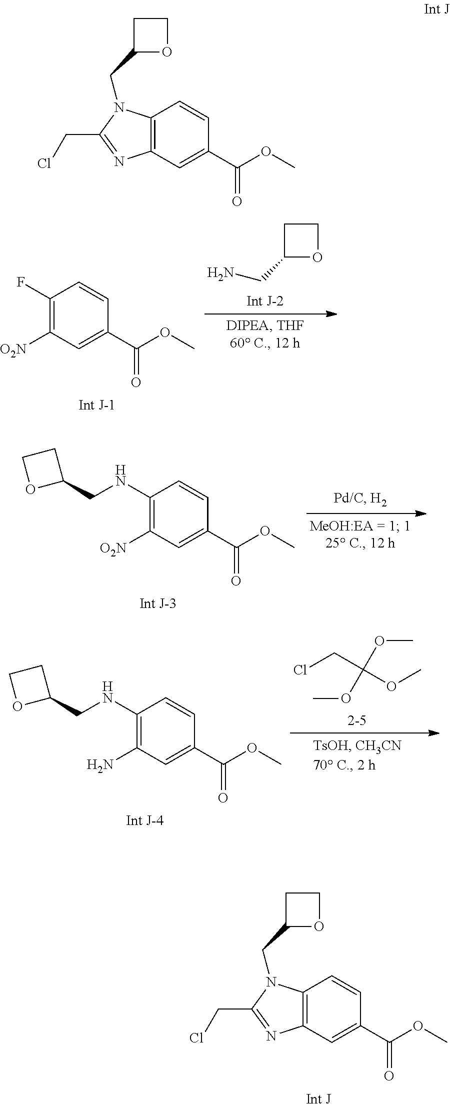

N-ethyl-N-isopropyl-propan-2-amine (25.9 g, 200.8 mmol) was added to a solution of [(2S)-oxetan-2-yl]methanamine (10.9 g, 125.5 mmol) and methyl 4-fluoro-3-nitrobenzoate (20.0 g, 100.5 mmol) in THF (100 ml) with stirring at 25° C. The reaction mixture was stirred at 60° C. for 4 h. the mixture was diluted with EtOAc (100 ml) and H₂O (200 ml). The layers were separated, and the aqueous layer was extracted with EtOAc (100 ml×2). The combined organic phase was washed with brine (200 ml) and dried over anhydrous Na₂SO₄, filtered and concentrated in vacuo. The crude product was slurried by methyl tert-butyl ether to obtain compound Int J-3 methyl (S)-3-nitro-4-((oxetan-2-ylmethyl)amino)benzoate (24.6 g, 92.3% yield) as a yellow solid. LCMS (ESI): calculated for C₁₂H₁₄N₂O₅; [M+H]⁺: 266.2, found: 267.2.

10% Pd/C (5.0 g) was added to a solution of compound Int J-3 (24.0 g, 90.1 mmol) in EtOAc:MeOH=1:1 (200 ml) with stirring at 25° C. The reaction mixture was stirred at 25° C. for 12 h under hydrogen. The mixture was filtered and the filtrate cake was washed with EtOAc (100 ml×2). The combined organic phase concentrated in vacuo to produce compound Int J-4 methyl (S)-3-amino-4-((oxetan-2-ylmethyl)amino)benzoate (18.8 g, 88.1% yield, 93% purity) as a brown solid. LCMS (ESI): calculated for C₁₂H₁₆N₂O₃; [M+H]⁺: 236.2, found: 237.2.

2-chloro-1,1,1-trimethoxyethane (14.1 g, 91.4 mmol) and 4-methylbenzenesulfonic acid (1.3 g, 7.62 mmol) were added to a stirred solution of compound Int J-4 methyl (S)-3-amino-4-((oxetan-2-ylmethyl)amino)benzoate (18.0 g, 76.2 mmol) in CH₃CN (150 ml) with stirring at RT. The reaction mixture was stirred at 70° C. for 2 h. The mixture was added EtOAc (100 ml) and H₂O (200 ml). The organic layers were separated, and the aqueous layer was extracted with EtOAc (100 ml×2). The combined organic phase was washed with brine (200 ml) and dried over anhydrous Na₂SO₄, filtered and concentrated in vacuo. The crude product was purified by flash column chromatography (petroleum ether/ethyl acetate 3:1) to produce compound Int J: methyl (S)-2-(chloromethyl)-1-(oxetan-2-ylmethyl)-1H-benzo[d]imidazole-5-carboxylate (18.0 g, 80% yield) as a yellow solid. LCMS (ESI): calculated for C₁₄H₁₅ClN₂O₃; [M+H]⁺: 294.7, found: 295.7.

### The Preparation of 6-((4-chloro-2-fluorobenzyl)oxy)-1′,2′,3′,6′-tetrahydro-2,4′-bipyridine (Int K) and 2-((4-chloro-2-fluorobenzyl)oxy)-6-(piperidin-4-yl)pyridine (Int L)

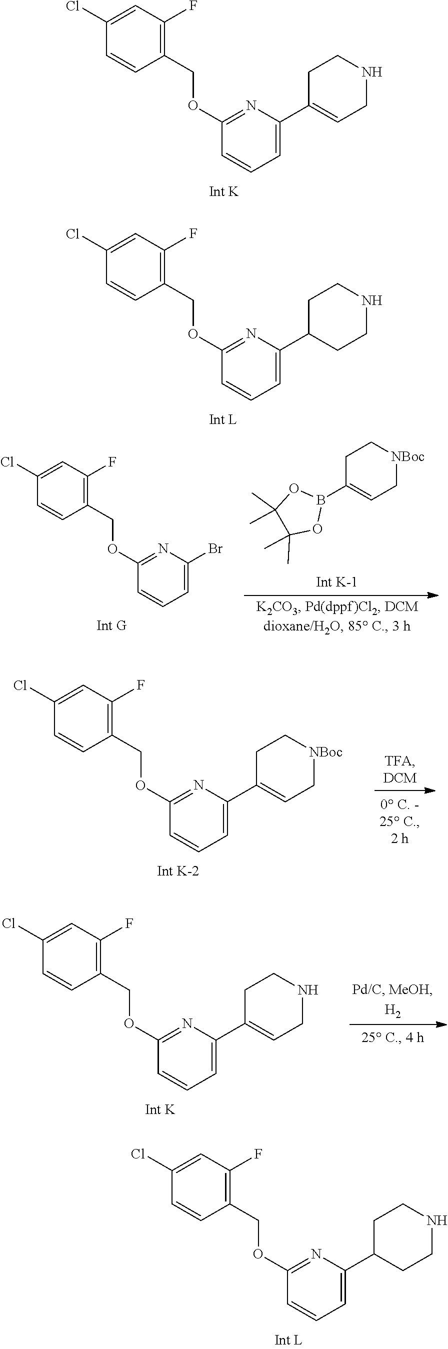

Tert-butyl 4-(4,4,5,5-tetramethyl-1,3,2-dioxaborolan-2-yl)-3,6-dihydropyridine-1(2H)-carboxylate (27.3 g, 88.2 mmol, 1.2 eq), K₂CO₃ (20.3 g, 147 mmol, 2.0 eq), Pd(dppf)Cl₂·DCM (5.7 g, 6.3 mmol, 0.085 eq), H₂O (40 ml) were added to a 500 ml round-bottom flask. Compound Int G (20.0 g, 73.5 mmol, 1.0 eq) in dioxane (200 ml) was added. The final mixture was maintained under N₂ atmosphere and stirred at 90° C. for 3.5 h. Then the reaction mixture was cooled down to RT and filtered, the filter cake was washed with EA (100 ml). Brine (200 ml) was added to the filtrate and separated, the aqueous phase was extracted with EA (100 ml×2), the combined organic phase was concentrated under vacuum. The residue was purified by recrystallization from PE:EA=1;1 to produce the crude compound, then was discolored with activated carbon at room temperature for 2 h. The mixture was filtered through a pad of celite, the filter cake was washed with EA then concentrated under vacuum to afford the crude compound. The crude compound Int K-2 was purified by recrystallized with EtOH/H₂O=6/1 to produce the desired product, tert-butyl 6-((4-chloro-2-fluorobenzyl)oxy)-3′,6′-dihydro-[2,4′-bipyridine]-1′(2′H)-carboxylate (20.1 g, yield 65%) as a light yellow solid. LCMS (ESI): calculated for C₂₂H₂₄ClFN₂O₃; [M+H]⁺: 418.8, found: 419.1.

Compound Int K-2 (20.0 g, 47.8 mmol) in DCM (100 ml) was added to a 500 ml round-bottom flask were added, then added TFA (trifluoroacetic acid) (20 ml), the reaction mixture was stirred at 25° C. for 2 hours. The reaction mixture was diluted with DCM, the reaction mixture was washed sequentially with aqueous sodium bicarbonate solution, water, and saturated aqueous sodium chloride solution, dried over sodium sulfate, filtered, and concentrated in vacuo. The crude compound 2-12 was purified by recrystallized with CH₃CN to afford desired product, Int K: 6-((4-chloro-2-fluorobenzyl)oxy)-1′,2′,3′,6′-tetrahydro-2,4′-bipyridine (13.5 g, yield 88%) as a white solid. LCMS (ESI): calculated for C₁₇H₁₆ClFN₂O; [M+H]⁺: 318.8, found: 319.1.

10% Pd/C (3.9 g) was added to a solution of compound Int K (13.0 g, 40.8 mmol) in EtOAc:MeOH=1:1 (100 ml) with stirring at 25° C. was added. The reaction mixture was stirred at 25° C. for 6 h under hydrogen. The mixture was filtered and the filtrate cake was washed with EtOAc (100 ml×2). The combined organic phase concentrated in vacuo to afford compound Int L: 2-((4-chloro-2-fluorobenzyl)oxy)-6-(piperidin-4-yl)pyridine (12.0 g, 91% yield) as a white solid. LCMS (ESI): calculated for C₁₇H₁₈ClFN₂O; [M+H]⁺: 320.8, found: 321.1.

### The Preparation of 4-(2-(4-chloro-2-fluorophenyl)-2-methylbenzo [d][1,3]dioxol-4-yl)-1,2,3,6-tetrahydropyridine (Int M)

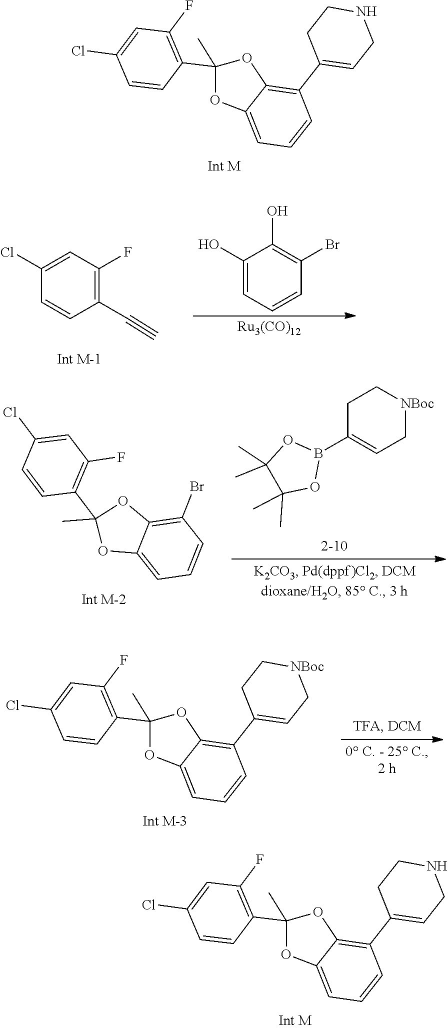

Under N₂ atmosphere, a mixture of 5-chloro-2-ethynylpyridine (1.80 g, 13.1 mmol), 3-bromobenzene-1, 2-diol (2.47 g, 13.1 mmol), and triruthenium dodecacarbonyl (167 mg, 0.261 mmol) in toluene (25 ml), and heated at 100° C. for 16 hours. The reaction mixture was diluted with ethyl acetate (30 ml) and filtered through a pad of diatomaceous earth; the filtrate was concentrated in vacuo and purified using silica gel chromatography (Gradient: 0% to 1% ethyl acetate in petroleum ether) to provide Int M-2 as a yellow oil, (1.5 g, 37.5%).

Tert-butyl 4-(4,4,5,5-tetramethyl-1,3,2-dioxaborolan-2-yl)-3,6-dihydropyridine-1(2H)-carboxylate (2.17 g, 8.8 mmol, 1.2 eq), K₂CO₃ (1.6 g, 11.7 mmol, 2.0 eq), Pd(dppf)Cl₂·DCM (0.4 g, 0.50 mmol, 0.085 eq), H₂O (4 ml) were added to compound Int M-2 (2.0 g, 5.84 mmol, 1.0 eq) in dioxane (20 ml) was added, the final mixture was charged under N² atmosphere and stirred at 90° C. for 3.5 h. Then the reaction mixture was cooled down to room temperature and filtered, the filter cake was washed with EA (20 ml). The brine (20 ml) was added to the filtrate and separated, the aqueous phase was extracted with EA (20 ml×2), the combined organic phase was concentrated under vacuum. The residue was purified by recrystallization from PE:EA=1:1 to afford the compound Int M-3 (2.1 g, 80.9%) as a light yellow solid. LCMS (ESI): calculated for C₂₄H₂₅ClFNO₄; [M+H]⁺: 446.15, found: 446.15.

Compound Int M-3 (2.0 g, 4.5 mmol) and DCM (10 ml) were added to a 50 ml round-bottom flask, TFA (2 ml) was then added, the reaction mixture was stirred at 25° C. for 2 hours. The reaction mixture was diluted with DCM, the reaction mixture was washed sequentially with aqueous sodium bicarbonate solution, water, and saturated aqueous sodium chloride solution, dried over sodium sulfate, filtered, and concentrated in vacuo. The crude compound was purified by recrystallized with CH₃CN to produce desired product Int M (1.3 g, yield 83.8%) as a white solid. LCMS (ESI): calculated for C₁₉H₁₇ClFNO₂; [M+H]⁺: 346.1, found: 346.1.

### PROCEDURES

### Procedure 1. Preparation of 2-(4-(6-((4-cyano-2-fluorobenzyl)oxy)pyridin-2-yl)benzyl)-N-(4-hydroxyphenethyl)-1-(2-methoxyethyl)-1H-benzo[d]imidazole-5-carboxamide (1)

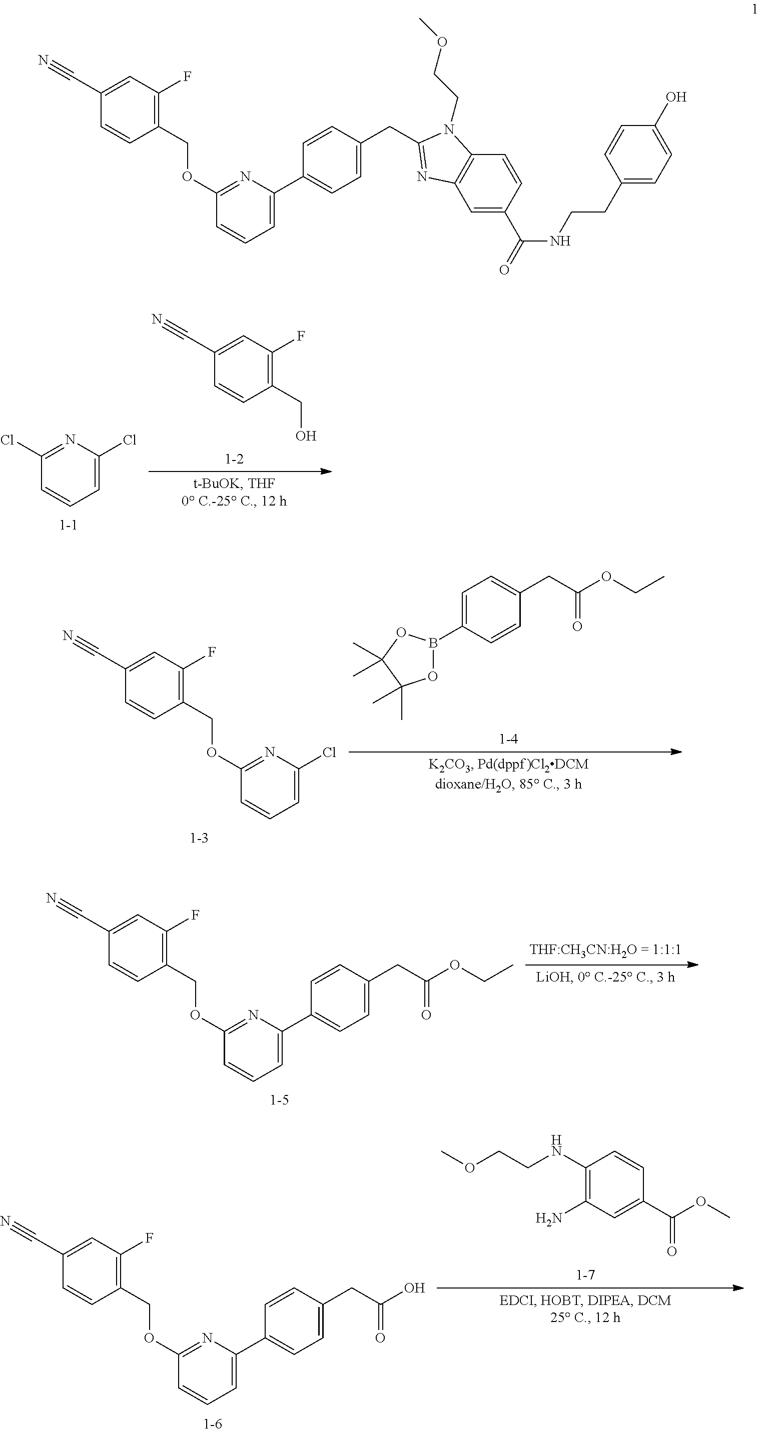

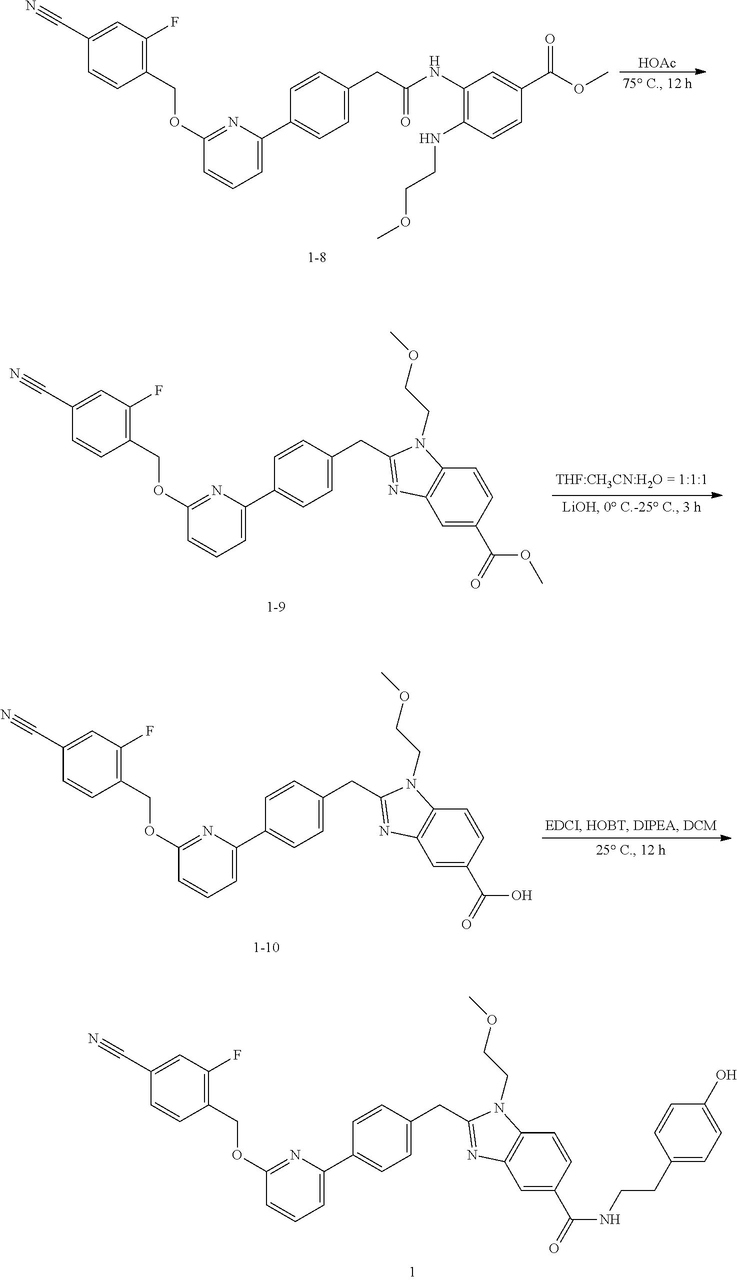 [SMILES: COCCNc1ccc(C(=O)OC)cc1NC(=O)Cc1ccc(-c2cccc(OCc3ccc(C#N)cc3F)n2)cc1 ; COCCn1c(Cc2ccc(-c3cccc(OCc4ccc(C#N)cc4F)n3)cc2)nc2cc(C(=O)OC)ccc21 ; "HOAc" ; COCCn1c(Cc2ccc(-c3cccc(OCc4ccc(C#N)cc4F)n3)cc2)nc2cc(C(=O)O)ccc21 ; COCCn1c(Cc2ccc(-c3cccc(OCc4ccc(C#N)cc4F)n3)cc2)nc2cc(C(=O)NCCc3ccc(O)cc3)ccc21]

Referring to the following reaction equation (Route 1): Potassium tert-butoxide (14.7 g, 0.13 mol, 2.4 eq) was added in THF (200 ml) in a 500 ml round-bottom flask and stirred for 5 min, 3-fluoro-4-(hydroxymethyl)benzonitrile (14 g, 0.092 mol, 1.7 eq) was then added and stirred for 45 min at 15° C. 2,6-dichloropyridine (8.07 g, 0.055 mol, 1.0 eq) was added into the mixture solution, and stirred for 18 h at 15° C. The saturated ammonium chloride aqueous solution (18 eq) and EA (100 ml) was added to work-up the reaction. The reaction mixture was filtered. The filtrate was extracted with EA (200 ml×2) and the combined organic phase was washed with brine (200 ml), dried over anhydrous sodium sulfate, filtered, the filtrate was concentrated under vacuum. The residue was purified by recrystallized with PE/EA=8/1 to produce crude compound 1-3. The crude compound 1-3 was purified by recrystallized with EtOH/H₂O=1/1 to produce the desired product (10.3 g, yield 70%) as a light yellow solid. LCMS (ESI): calculated for C₁₃H₈ClFN₂O; [M+H]⁺: 262.6, found: 263.6.

Compound 1-3 (10.0 g, 0.038 mol, 1.0 eq) in dioxane (100 ml, 10V) was added into a 250 ml round-bottom flask, ethyl 2-(4-(4,4,5,5-tetramethyl-1,3,2-dioxaborolan-2-yl)phenyl)acetate (14.4 g, 0.050 mol, 1.3 eq), K₂CO₃ (10.8 g, 0.760 mol, 2.0 eq), Pd(dppf)Cl₂·DCM (2.5 g, 0.003 mmol, 0.085 eq), and H₂O (20 ml) were then added to the flask. The final mixture was maintained under N₂ atmosphere and stirred at 90° C. for 3.5 h. The reaction mixture was then cooled down to RT and filtered, the filter cake was washed with EA (100 ml). Brine (100 ml) was added to the filtrate and separated, the aqueous phase was extracted with EA (100 ml×2), the combined organic phase was concentrated under vacuum. The residue was purified by recrystallization from PE:EA=1:1 to produce compound 1-5. The crude compound 1-5 was discolored with activated carbon at room temperature for 2 h. The mixture was filtered through a pad of celite, the filter cake was washed with EA then concentrated under vacuum and purified by recrystallized with EtOH/H₂O=6/1 to afford desired product 1-5 (8.8 g, yield 65%) as a light yellow solid. LCMS (ESI): calculated for C₂₃H₁₉FN₂O₃; [M+H]⁺: 390.4, found: 391.4.

Compound 1-5 (8.0 g, 20.49 mmol) into THF (20.0 ml), CH₃CN (20.0 ml), Water (20.0 ml) and lithium hydroxide (982 mg, 41 mmol) were added to a 250 ml round-bottom flask at 25° C., the reaction mixture was stirred at 25° C. for 12 hours. It was then concentrated to remove organic solvents, and the aqueous residue was adjusted to pH 6 by addition of 1 M hydrochloric acid. The resulting mixture was extracted with EA (60 ml×3), and the combined organic layers were dried over magnesium sulfate, filtered, and concentrated in vacuo to get crude product. The crude compound 1-6 was purified by recrystallized with EtOH to produce the desired product (6.5 g, yield 87%) as a light yellow solid. LCMS (ESI): calculated for C₂₁H₁₅FN₂O₃; [M+H]⁺: 362.3, found: 363.3.

A solution of compound 1-6 (6.0 g, 16.56 mmol), compound 1-7 (3.71 g, 16.56 mmol), and O-(7-Azabenzotriazol-1-yl)-N,N,N′,N′-tetraMethyluroniuM hexafluorophosphate (HATU; 9.45 g, 33.12 mmol) in N,N-dimethylformamide (60 ml) was stirred at 25° C. for 30 minutes, whereupon triethylamine (6.7 g, 66.23 mmol) was added, and stirring was continued at 25° C. for 12 h. The reaction mixture was then poured into water (300 ml) and extracted with ethyl acetate (200 ml×3). The combined organic layers were washed with saturated aqueous sodium chloride solution (200 ml×3), dried over sodium sulfate, filtered, and concentrated in vacuo. Silica gel chromatography (Eluent: 1:1 petroleum ether/ethyl acetate) produced compound 1-8 as a brown solid (7.8 g, yield 83%), which was taken directly into the following step. LCMS (ESI): calculated for C₃₂H2₉FN₄O₅; [M+H]⁺: 568.6, found: 569.6.

A solution of compound 1-8 (7.5 g, 13.19 mmol) in acetic acid (50 ml) was stirred at 75° C. for 12 hours, whereupon it was concentrated in vacuo to provide crude as a brown solid. The residue was purified by recrystallized with CH₃CN to produce the desired product compound 1-9 (6.1 g, yield 85%) as a yellow solid. LCMS (ESI): calculated for C₃₂H₂₇FN₄O₄; [M+H]⁺: 550.6, found: 551.6.

Compound 1-9 (6.0 g, 10.9 mmol) in THF (20.0 ml) was added to a 250 ml round-bottom flask at 0° C., CH₃CN (20.0 ml), water (20.0 ml) and lithium hydroxide (522 mg, 21.8 mmol) were then added, the reaction mixture was stirred at 25° C. for 12 h. It was then concentrated to remove organic solvents, and the aqueous residue was adjusted to pH 6 by addition of 1M hydrochloric acid. The resulting mixture was extracted with EA (60 ml×3), and the combined organic layers were dried over magnesium sulfate, filtered, and concentrated in vacuo to obtain a crude product. The crude compound 1-10 was purified by recrystallized with CH₃CN to afford desired product compound 1-10 (5.1 g, yield 87%) as a yellow solid. LCMS (ESI): calculated for C₃₁H₂₅FN₄O₄; [M+H]⁺: 536.5, found: 537.6.

A solution of compound 1-10 (100 mg, 1.86 mmol), N-Hydroxybenzotrizole (HOBT; 37.78 mg, 2.79 mmol), and N-(3-dimethylaminopropyl)-n″-ethylcarbodiimidehydrochloride (EDCI; 9.45 g, 33.12 mmol) in N,N-dimethylformamide (2 ml) was stirred at 25° C. for 30 minutes, whereupon N,N-Diisopropylethylamine (48.18 mg, 3.73 mmol) and 4-(2-aminoethyl)phenol (37.8 mg, 1.86 mmol) was added, and stirring was continued at 25° C. for 12 h. The reaction mixture was then poured into water (10 ml) and extracted with ethyl acetate (10 ml×3). The combined organic layers were washed with saturated aqueous sodium chloride solution (10 ml×3), dried over sodium sulfate, filtered, and concentrated in vacuo. The crude was purified by preparative liquid phase chromatography to produce compound 1 (67 mg, yield: 54.9%). LCMS (ESI): calculated for C₃₉H₃₄FN₅O₄; [M+H]⁺: 655.73, found: 656.2.

### Procedure 2. Preparation of (2S,4R)-1-((2-((4-(6-((4-chloro-2-fluorobenzyl)oxy)pyridin-2-yl)piperidin-1-yl)methyl)-1-(((S)-oxetan-2-yl)methyl)-1H-benzo[d]imidazole-5-carbonyl)glycyl)-4-hydroxypyrrolidine-2-carboxylic acid (2)

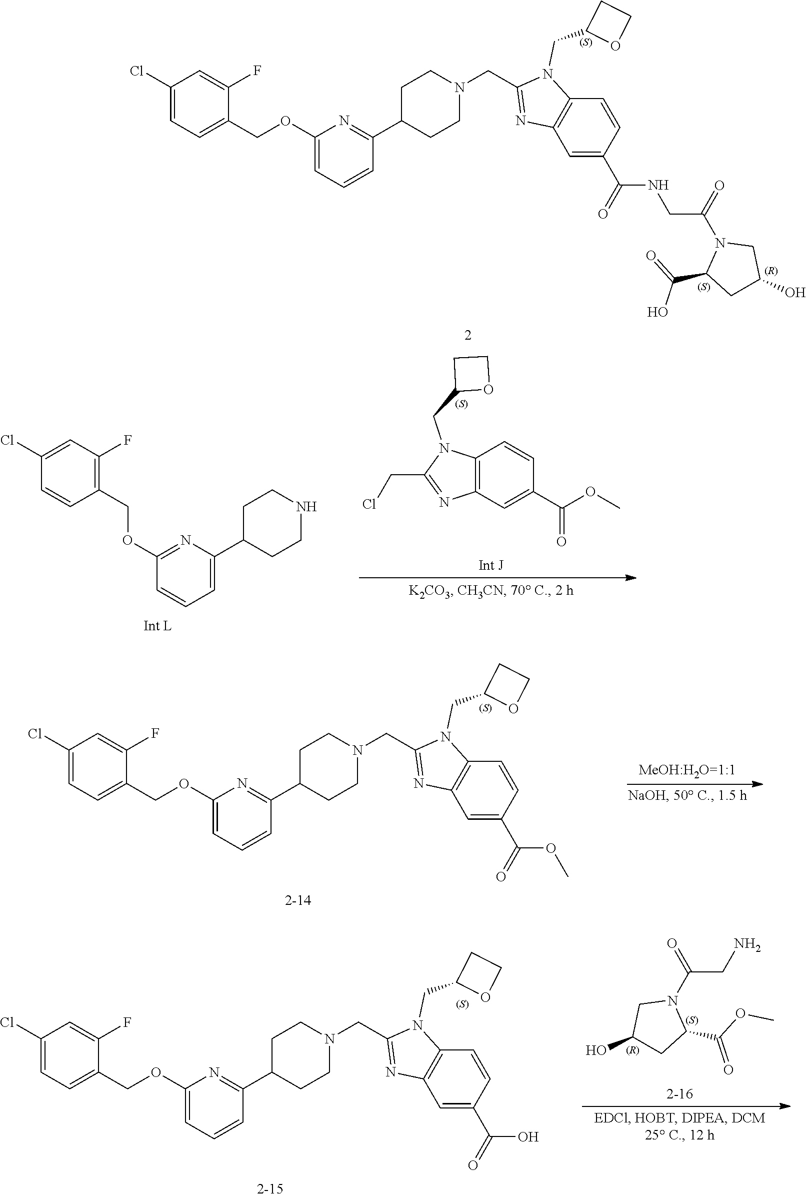

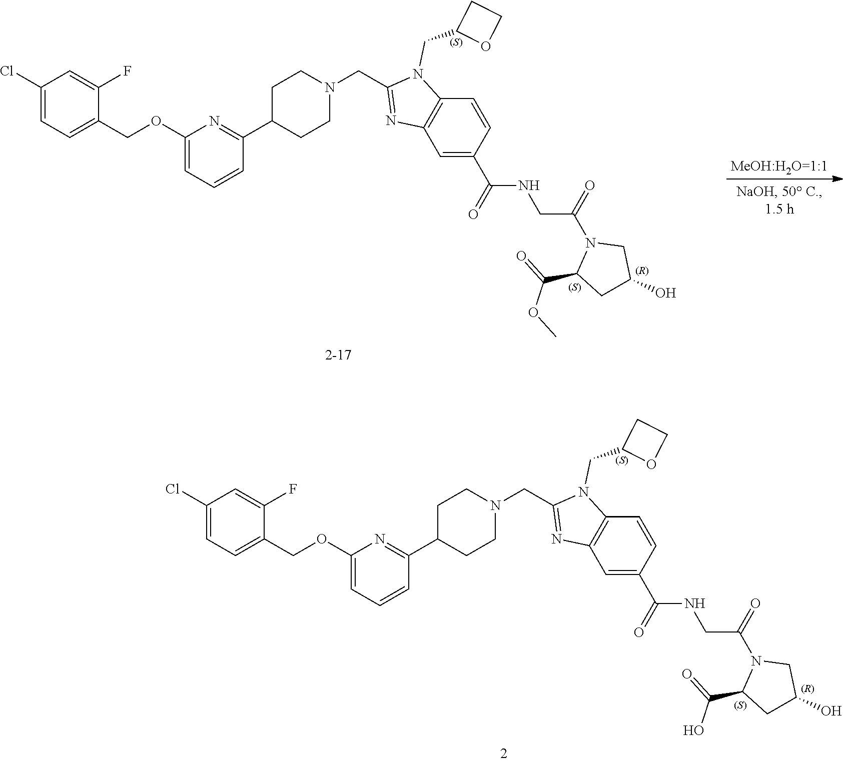

Referring to the following reaction equation (Route 2):

Compound Int J (12.2 g, 41.2 mmol) was added to a solution of compound Int L (12.0 g, 37.4 mmol) in acetonitrile (120 ml) with stirring, followed by addition of potassium carbonate (15.5 g, 112 mmol). The reaction mixture was stirred at 75° C. for 3 h. The reaction mixture was filtered and washed with EA (100 ml), the filtrate was concentrated in vacuo, the crude product was purified by slurry (PE:EA=5:1) to produce compound 2-14, methyl (S)-2-((4-(6-((4-chloro-2-fluorobenzyl)oxy)pyridin-2-yl)piperidin-1-yl)methyl)-1-(oxetan-2-ylmethyl)-1H-benzo[d]imidazole-5-carboxylate (17.5 g, 82% yield) as a yellow solid. LCMS (ESI): calculated for C₃₁H₃₂ClFN₄O₄; [M+H]⁺: 579.1, found: 579.2.

Compound 2-14 (16.0 g, 10.9 mmol) into MeOH (80.0 ml), water (80.0 ml) and sodium hydroxide (5.5 g, 138.0 mmol) were added to a 250 ml round-bottom flask at 0° C., the reaction mixture was stirred at 50° C. for 2 h. It was then concentrated to remove organic solvents, and the aqueous residue was adjusted to pH 6 by addition of 1M hydrochloric acid. The resulting mixture was extracted with EA (100 ml×3), and the combined organic layers were dried over magnesium sulfate, filtered, and concentrated in vacuo to get crude product. The crude product was purified by slurried with CH₃CN to produce the desired product compound 2-15, (S)-2-((4-(6-((4-chloro-2-fluorobenzyl)oxy)pyridin-2-yl)piperidin-1-yl)methyl)-1-(oxetan-2-ylmethyl)-1H-benzo[d]imidazole-5-carboxylic acid (13.5 g, yield 84%) as a yellow solid. LCMS (ESI): calculated for C₃₀H₃₀ClFN₄O₄; [M+H]⁺: 565.1, found: 565.2.

A solution of compound 2-15 (100 mg, 0.177 mmol) from above, N-Hydroxybenzotrizole (HOBT; 35.8 mg, 0.265 mmol), and N-(3-dimethylaminopropyl)-n″-ethylcarbodiimidehydrochloride (EDCI; 50.9 mg, 0.265 mmol) in N,N-dimethylformamide (2 mL) was stirred at 25° C. for 30 minutes, whereupon N,N-Diisopropylethylamine (58.0 mg, 0.442 mmol) and compound 2-16 methyl (2S,4R)-1-glycyl-4-hydroxypyrrolidine-2-carboxylate (40.0 mg, 0.195 mmol) was added, and stirring was continued at 25° C. for 12 h. The reaction mixture was then poured into water (10 ml) and extracted with ethyl acetate (10 ml×3). The combined organic layers were washed with saturated aqueous sodium chloride solution (10 ml×2), dried over sodium sulfate, filtered, and concentrated in vacuo. The crude was purified by preparative liquid phase chromatography to give compound 2-17, methyl (2S,4R)-1-((2-((4-(6-((4-chloro-2-fluorobenzyl)oxy)pyridin-2-yl)piperidin-1-yl)methyl)-1-(((S)-oxetan-2-yl)methyl)-1H-benzo[d]imidazole-5-carbonyl)glycyl)-4-hydroxypyrrolidine-2-carboxylate (67 mg, yield: 51%) as a white solid. LCMS (ESI): calculated for C₃₈H₄₂ClFN₆O₇; [M+H]+: 749.2, found: 749.2.

Compound 2-17 (60.0 mg, 0.08 mmol) in MeOH (1.0 ml), water (1.0 ml) and sodium hydroxide (16 mg, 0.40 mmol) were added to a 25 ml round-bottom flask at 0° C., the reaction mixture was stirred at 50° C. for 1.5 h. It was then concentrated to remove organic solvents, and the aqueous residue was adjusted to pH 6 by addition of 1M hydrochloric acid. The resulting mixture was extracted with EA (10 ml×3), and the combined organic layers were dried over magnesium sulfate, filtered, and concentrated in vacuo to get crude product. The crude product was purified by preparative liquid phase chromatography to afford desired product compound 2, (2S,4R)-1-((2-((4-(6-((4-chloro-2-fluorobenzyl)oxy)pyridin-2-yl)piperidin-1-yl)methyl)-1-(((S)-oxetan-2-yl)methyl)-1H-benzo[d]imidazole-5-carbonyl)glycyl)-4-hydroxypyrrolidine-2-carboxylic acid (35 mg, yield 60%) as a white solid. LCMS (ESI): calculated for C₃₇H₄₀ClFN₆O₇; [M+H]⁺: 735.2, found: 735.2. ¹H NMR (400 MHz, DMSO-d₆).

### Procedure 3. Preparation of (S)-2-((6-((4-chloro-2-fluorobenzyl)oxy)-3′,6′-dihydro-[2,4′-bipyridin]-1′(2′H)-yl)methyl)-N-(3-methyl-1H-indazol-5-yl)-1-(oxetan-2-ylmethyl)-1H-benzo[d]imidazole-5-carboxamide (3)

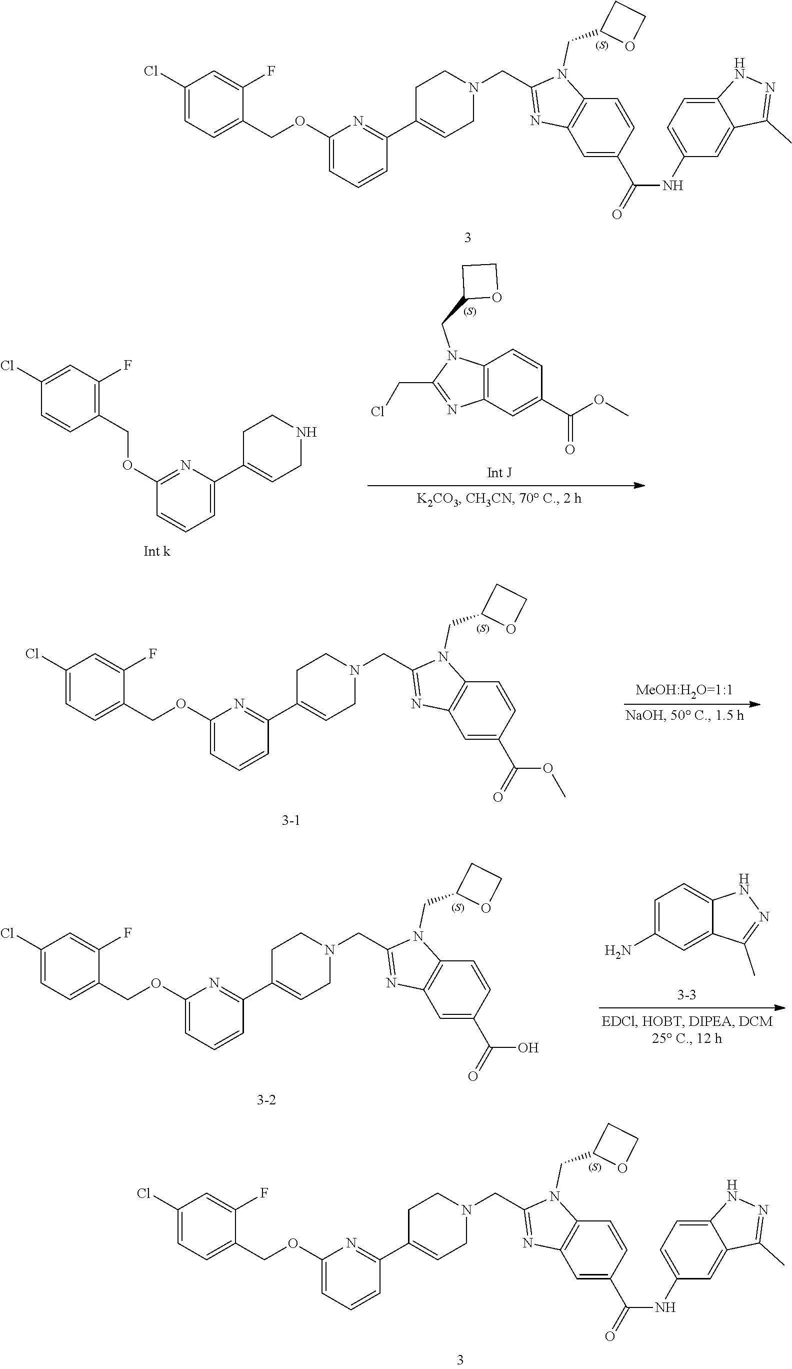

Referring to the following reaction equation (Route 3): compound Int J (10.2 g, 34 mmol) was added to a solution of compound Int K (10.0 g, 31.3 mmol) in acetonitrile (100 ml) with stirring, followed by the addition of potassium carbonate (10.8 g, 78.4 mmol). The reaction mixture was stirred at 75° C. for 3 h. The reaction mixture was filtered and washed with EA (100 ml), the filtrate was concentrated in vacuo, the crude product was purified by slurry (PE:EA=5:1) to produce compound 3-1 methyl (S)-2-((6-((4-chloro-2-fluorobenzyl)oxy)-3′,6′-dihydro-[2,4′-bipyridin]-1′(2′H)-yl)methyl)-1-(oxetan-2-ylmethyl)-1H-benzo[d]imidazole-5-carboxylate (14.5 g, 80% yield) as a yellow solid. LCMS (ESI): calculated for C₃₁H₃₀ClFN₄O₄; [M+H]⁺: 577.1, found: 577.1.

Compound 3-1 (14.0 g, 24.3 mmol) into MeOH (70.0 ml), water (70.0 ml) and sodium hydroxide (4.85 g, 121.3 mmol) were added to a 250 ml round-bottom flask at 0° C., the reaction mixture was stirred at 50° C. for 2 hours. It was then concentrated to remove organic solvents, and the aqueous residue was adjusted to pH 6 by addition of 1M hydrochloric acid. The resulting mixture was extracted with EA (100 ml×3), and the combined organic layers were dried over magnesium sulfate, filtered, and concentrated in vacuo to get crude product. The crude compound 3-2 was purified by slurried with CH₃CN to produce desired product, (S)-2-((6-((4-chloro-2-fluorobenzyl)oxy)-3′,6′-dihydro-[2,4′-bipyridin]-1′(2′H)-yl)methyl)-1-(oxetan-2-ylmethyl)-1H-benzo[d]imidazole-5-carboxylic acid (11.7 g, yield 86%) as a yellow solid. LCMS (ESI): calculated for C₃₀H₂₈ClFN₄O₄; [M+H]⁺: 563.0, found: 563.1.

A solution of compound 3-2 (100 mg, 0.177 mmol), N-Hydroxybenzotrizole (HOBT; 35. mg, 0.265 mmol), and N-(3-dimethylaminopropyl)-n″-ethylcarbodiimidehydrochloride (EDCI; 50.9 mg, 0.265 mmol) in N,N-dimethylformamide (2 ml) was stirred at 25° C. for 30 minutes, whereupon N,N-Diisopropylethylamine (58.0 mg, 0.442 mmol) and 3-methyl-1H-indazol-5-amine (28.8 mg, 0.195 mmol) was added, and stirring was continued at 25° C. for 12 h. The reaction mixture was then poured into water (10 ml) and extracted with ethyl acetate (10 ml×3). The combined organic layers were washed with saturated aqueous sodium chloride solution (10 ml×2), dried over sodium sulfate, filtered, and concentrated in vacuo. The crude was purified by preparative liquid phase chromatography to give compound 3, (S)-2-((6-((4-chloro-2-fluorobenzyl)oxy)-3′,6′-dihydro-[2,4′-bipyridin]-1′(2′H)-yl)methyl)-N-(3-methyl-1H-indazol-5-yl)-1-(oxetan-2-ylmethyl)-1H-benzo[d]imidazole-5-carboxamide (64 mg, yield: 52%) as a white solid. LCMS (ESI): calculated for C₃₈H₃₅ClFN₇O₃; [M+H]⁺: 692.1, found: 692.2. ¹H NMR (400 MHz, DMSO-d₆).

### Procedure 4. Preparation of (S)—N-(2-((6-((4-chloro-2-fluorobenzyl)oxy)-3′,6′-dihydro-[2,4′-bipyridin]-1′(2′H)-yl)methyl)-1-(oxetan-2-ylmethyl)-1H-benzo[d]imidazol-5-yl)-3-methyl-1H-indazole-5-carboxamide (4)

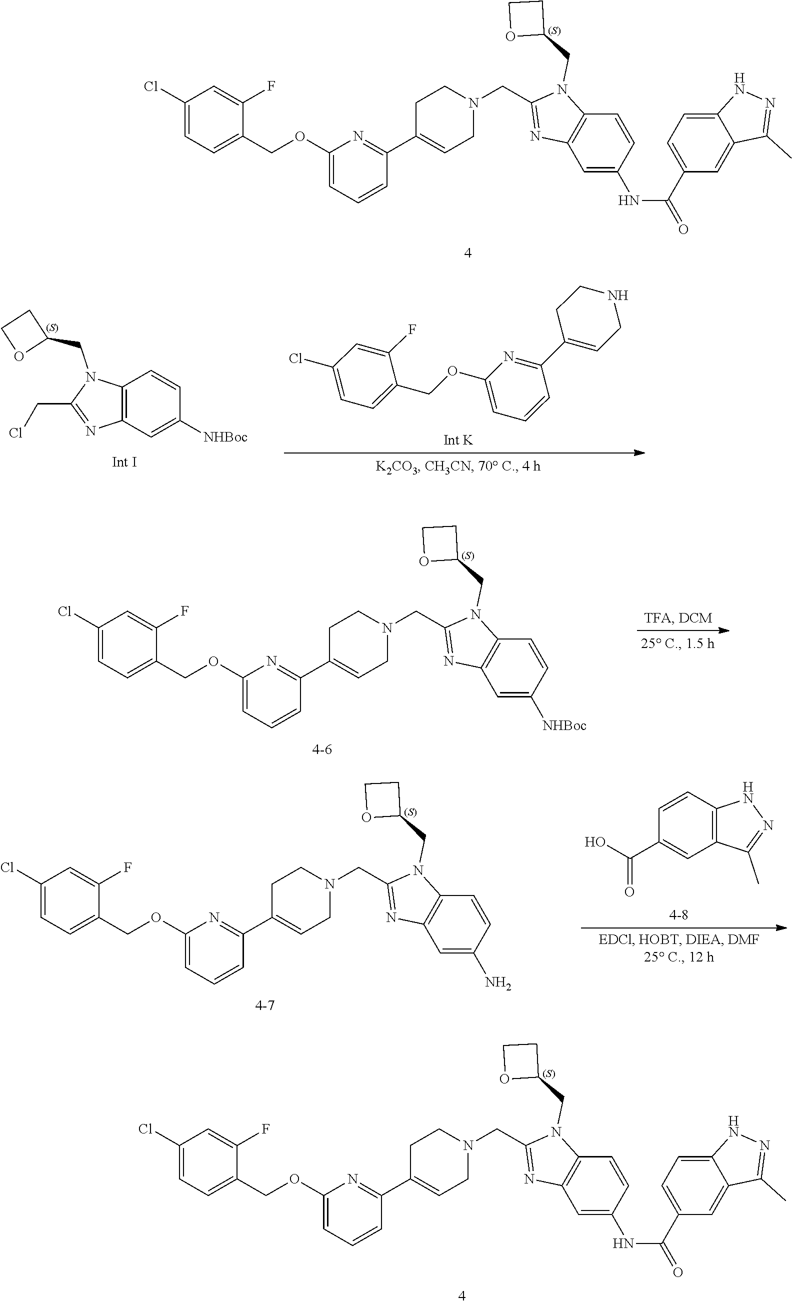

Referring to the following reaction equation (Route 4):

Compound Int K (17.0 g, 48.3 mmol) was added to a solution of compound Int I (14.0 g, 43.9 mmol) in acetonitrile (100 ml) with stirring, followed by addition of potassium carbonate (15.2 g, 109.8 mmol). The reaction mixture was stirred at temperature 75° C. for 3 h. The reaction mixture was filtered and washed with EA (100 ml), the filtrate was concentrated in vacuo, the crude product was purified by slurry (PE:EA=6:1) to produce compound 4-6 tert-butyl (S)-(2-((6-((4-chloro-2-fluorobenzyl)oxy)-3′,6′-dihydro-[2,4′-bipyridin]-1′(2′H)-yl)methyl)-1-(oxetan-2-ylmethyl)-1H-benzo[d]imidazol-5-yl)carbamate (22.5 g, 80.60 yield) as an off-white solid. LCMS (ESI): calculated for C₃₄H₃₇ClFN₅O₄; [M+H]⁺: 634.1, found: 634.2.

Compound 4-6 (20.0 g, 31.5 mmol) in DCM (100.0 ml) were added to a 500 ml round-bottom flask, TFA (20 ml) was then added to the flask, the reaction mixture was stirred at 25° C. for 2 h. The mixture was diluted with DCM, the reaction mixture was washed sequentially with aqueous sodium bicarbonate solution, water, and saturated aqueous sodium chloride solution, dried over sodium sulfate, filtered, and concentrated in vacuo. The crude product was purified by recrystallized with PE:EA=5:1 to produce the desired product compound 4-7, (S)-2-((6-((4-chloro-2-fluorobenzyl)oxy)-3′,6′-dihydro-[2,4′-bipyridin]-1′(2′H)-yl)methyl)-1-(oxetan-2-ylmethyl)-1H-benzo[d]imidazol-5-amine (13.5 g, yield 80%) as an off-white solid. LCMS (ESI): calculated for C₂₉H₂₉ClFN₅O₂; [M+H]⁺: 534.1, found: 534.2.

A solution of compound 4-8 (40 mg, 0.227 mmol), N-Hydroxybenzotrizole (HOBT; 46.0 mg, 0.340 mmol), and N-(3-dimethylaminopropyl)-n″-ethylcarbodiimidehydrochloride (EDCI; 65.1 mg, 0.340 mmol) in N,N-dimethylformamide (2 ml) was stirred at 25° C. for 30 minutes, whereupon N,N-Diisopropylethylamine (58.0 mg, 0.442 mmol) and compound 4-7 (121.1 mg, 0.227 mmol) were added, and stirred at 25° C. for 12 hours. The reaction mixture was then poured into water (10 ml) and extracted with ethyl acetate (10 ml×3). The combined organic layers were washed with saturated aqueous sodium chloride solution (10 ml×2), dried over sodium sulfate, filtered, and concentrated in vacuo. The crude was purified by preparative liquid phase chromatography to give compound 4, (S)—N-(2-((6-((4-chloro-2-fluorobenzyl)oxy)-3′,6′-dihydro-[2,4′-bipyridin]-1′(2′H)-yl)methyl)-1-(oxetan-2-ylmethyl)-1H-benzo[d]imidazol-5-yl)-3-methyl-1H-indazole-5-carboxamide, 81 mg, yield: 51.9% as a white solid. LCMS (ESI): calculated for C₃₈H₃₅ClFN₇O₃; [M+H]⁺: 692.1, found: 692.2.

### Procedure 5. Preparation of 2-((4-((S)-2-(4-chloro-2-fluorophenyl)-2-methylbenzo[d][1,3]dioxol-4-yl)-3,6-dihydropyridin-1(2H)-yl)methyl)-N-(3-methyl-1H-indazol-5-yl)-1-(((S)-oxetan-2-yl)methyl)-1H-benzo[d]imidazole-5-carboxamide (5)

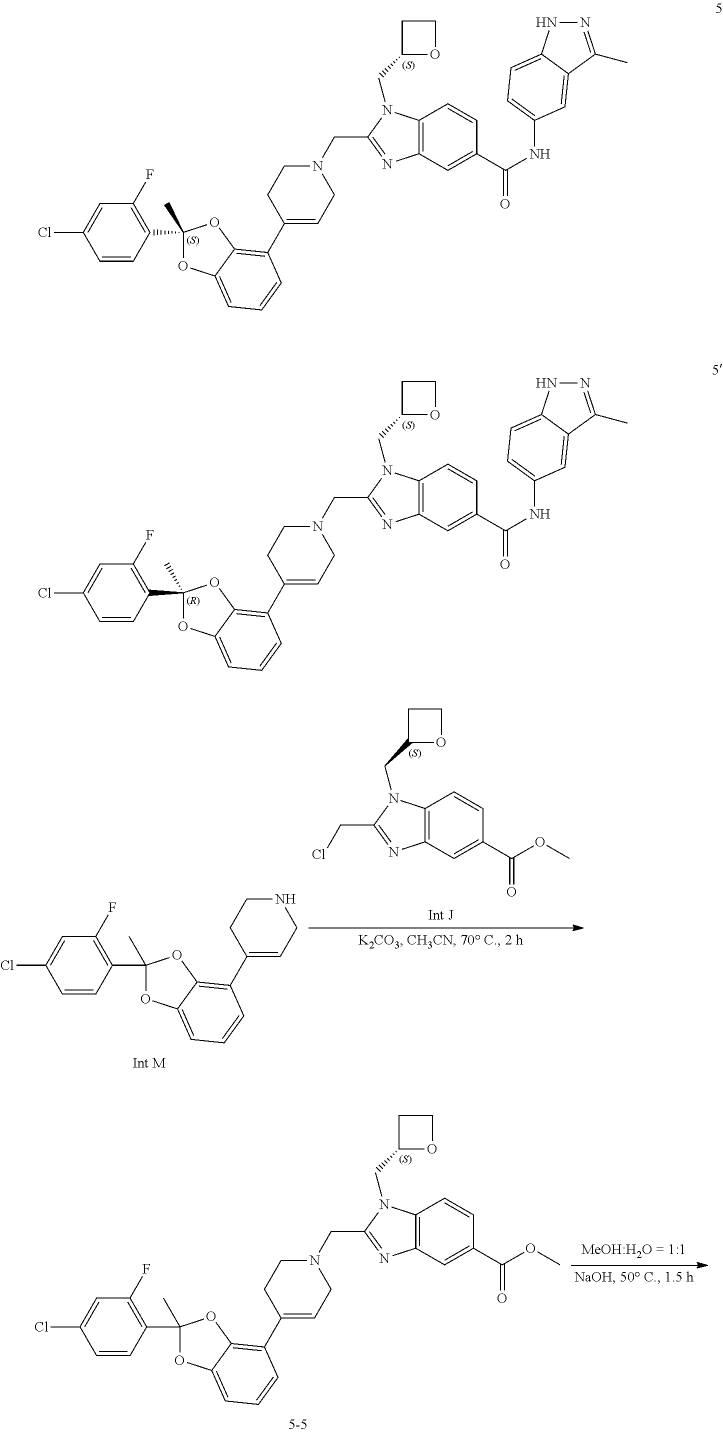

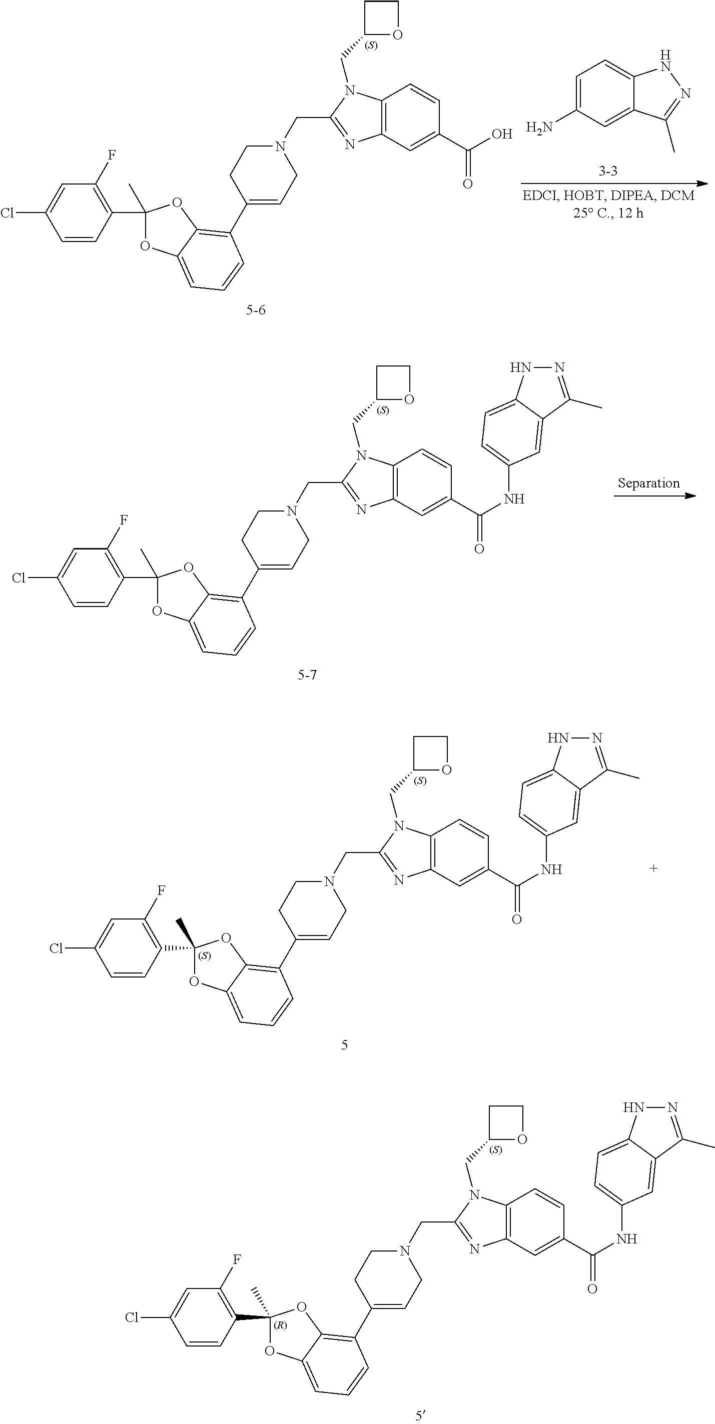

Referring to the following reaction equation (Route 5):

Compound Int J (1.0 g, 3.48 mmol) was added to a solution of compound Int M (1.0 g, 2.9 mmol) in acetonitrile (10 ml) with stirring, followed by addition of potassium carbonate (10.5 g, 7.25 mmol) and the reaction mixture was stirred at 75 C for 3 h. The reaction mixture was filtered and washed with EA (20 ml), the filtrate was concentrated in vacuo, the crude product was purified by slurry (PE:EA=5:1) to produce compound 5-5 (1.5 g, 85.8% yield) as a yellow solid. LCMS (ESI): calculated for C₃₃H₃₁ClFN₃O₅; [M+H]⁺: 604.2, found: 604.2.

Compound 5-5 (1.4 g, 2.3 mmol), MeOH (7.0 ml), water (7.0 ml) and sodium hydroxide (0.46 g, 11.5 mmol) were added into a 25 ml round-bottom flask at 0° C., the reaction mixture was stirred at 50° C. for 2 h, then concentrated to remove organic solvents. The aqueous residue was adjusted to pH 6 by addition of 1M hydrochloric acid. The resulting mixture was extracted with EA (10 ml×3), and the combined organic layers were dried over magnesium sulfate, filtered, and concentrated in vacuo to produce a crude product. The crude compound 5-6 was purified by slurried with CH₃CN to afford desired product (1.2 g, yield 87.8%) as a yellow solid. LCMS (ESI): calculated for C₃₂H₂₉ClFN₃O₅; [M+H]⁺: 590.2, found: 590.2.

A solution of compound 5-6 (100 mg, 0.17 mmol), N-Hydroxybenzotrizole (HOBT; 35.8 mg, 0.265 mmol), and N-(3-dimethylaminopropyl)-n″-ethylcarbodiimidehydrochloride (EDCI; 50.9 mg, 0.265 mmol) in N,N-dimethylformamide (2 ml) was stirred at 25° C. for 30 minutes, whereupon N,N-Diisopropylethylamine (58.0 mg, 0.442 mmol) and 3-methyl-1H-indazol-5-amine (28.8 mg, 0.195 mmol) were added, and stirring was continued at 25° C. for 12 h. The reaction mixture was then poured into water (10 ml) and extracted with ethyl acetate (10 ml×3). The combined organic layers were washed with saturated aqueous sodium chloride solution (10 ml×2), dried over sodium sulfate, filtered, and concentrated in vacuo. The crude was purified by preparative liquid phase chromatography to produce a mixture of the diastereomeric products 5-7. Separation of the two products was carried out via SFC (Supercritical Fluid Chromatography) [Column: Chiral Technologies ChiralCel OD, 5 pm; Mobile phase: 55:45 carbon dioxide/(methanol containing 0.1% ammonium hydroxide)], to produce two chiral compounds: compound 5, 23 mg, LCMS (ESI): calculated for C₄₀H₃₆ClFN₆O₄; [M+H]⁺: 719.3, found: 719.3; and compound 5′, 19 mg, LCMS (ESI): calculated for C₄₀H₃₆ClFN₆O₄; [M+H]⁺: 719.3, found: 719.3.

### Procedure 6. Preparation of 2-(6-(6-((4-cyano-2-fluorobenzyl)oxy)pyridin-2-yl)-6-azaspiro[2.5]octan-1-yl)-1-(2-methoxyethyl)-N-(3-methyl-1H-indazol-5-yl)-1H-benzo[d]imidazole-5-carboxamide (6)

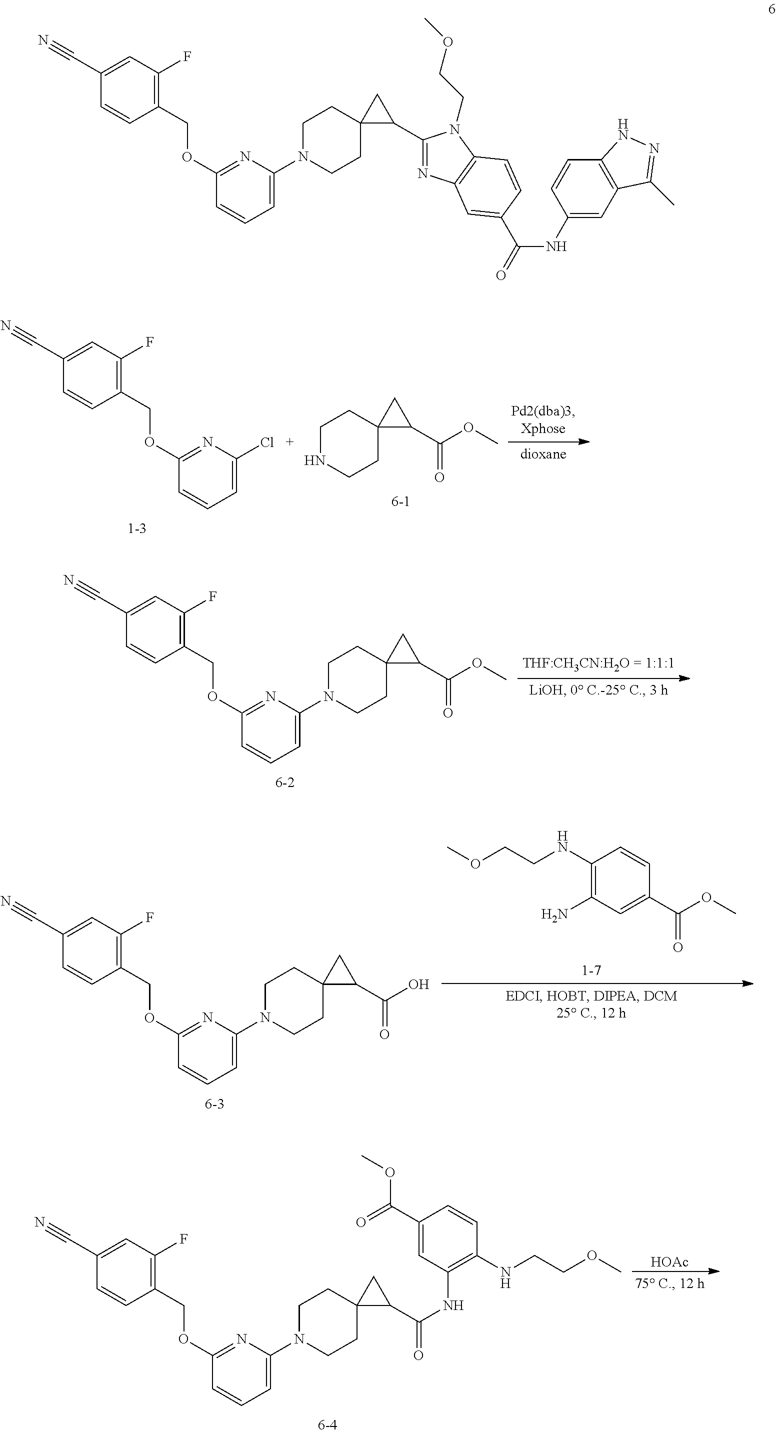

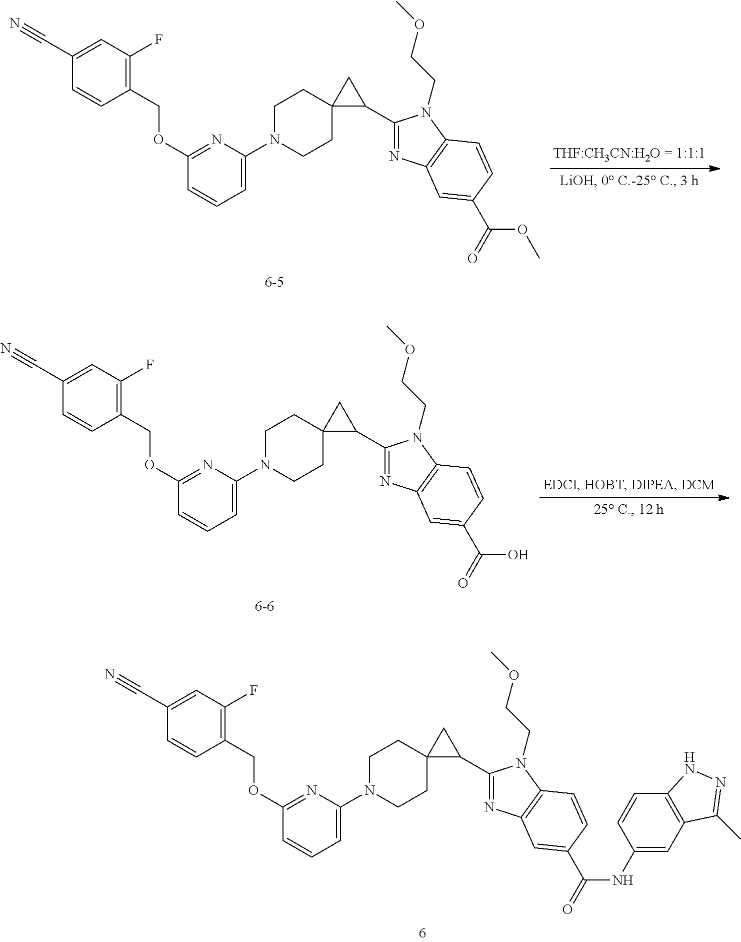 [SMILES: COCCn1c(C2CC23CCN(c2cccc(OCc4ccc(C#N)cc4F)n2)CC3)nc2cc(C(=O)OC)ccc21 ; COCCn1c(C2CC23CCN(c2cccc(OCc4ccc(C#N)cc4F)n2)CC3)nc2cc(C(=O)O)ccc21 ; COCCn1c(C2CC23CCN(c2cccc(OCc4ccc(C#N)cc4F)n2)CC3)nc2cc(C(=O)Nc3ccc4[nH]nc(C)c4c3)ccc21]

Under N₂ atmosphere, a mixture of compound 1-3 (2.0 g, 7.61 mmol), methyl 6-azaspiro[2.5]octane-1-carboxylate (1.42 g, 8.38 mmol), Cs₂CO₃ (4.97 g, 15.2 mmol), Pd2(dba)3 (696 mg, 0.77 mmol) and X-Phose (734 mg, 1.54 mmol) in dioxane (20 ml) was prepared, heated at 100° C. for 4 h. The reaction mixture was diluted with ethyl acetate (30 ml) and filtered through a pad of diatomaceous earth; the filtrate was concentrated in vacuo and purified using silica gel chromatography (Gradient: 0% to 5% MeOH in dichloromethane) to provide compound 6-2 as a yellow oil, (1.5 g, 49%).

The next series of reactions can follow the Procedure 1. After the Hydrolysis, condensation reaction, cyclization, hydrolysis and condensation to obtain compound 6.

The following compounds were prepared according to the procedures described herein (and indicated in Table 1 under Procedure) using the appropriate starting material(s) and appropriate protecting group chemistry as needed.

TABLE 1
The List of Compounds

| No. | Structure |
|---|---|
| 1 | 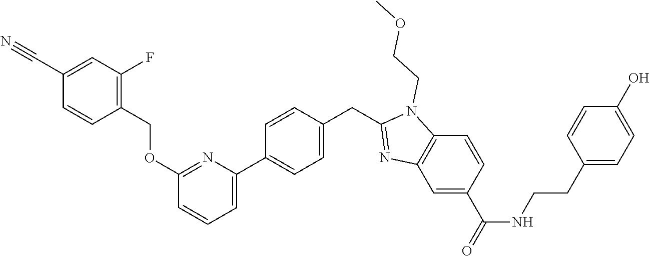 [SMILES: COCCn1c(Cc2ccc(-c3cccc(OCc4ccc(C#N)cc4F)n3)cc2)nc2cc(C(=O)NCCc3ccc(O)cc3)ccc21] |
| 2 | 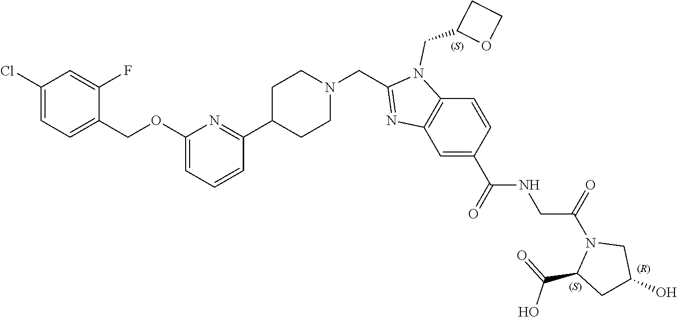 |
| 3 | 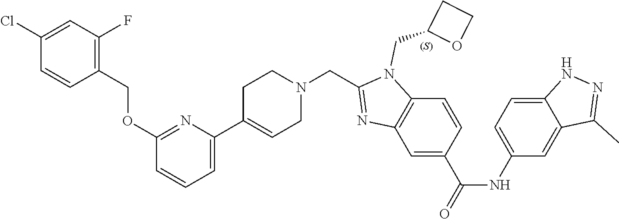 |
| 4 | 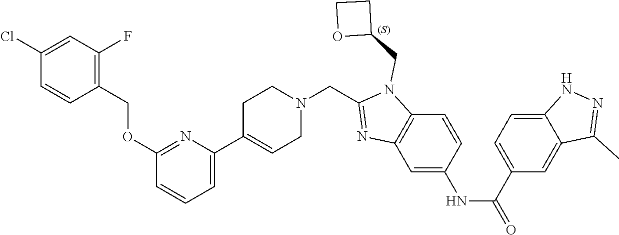 |
| 5 | 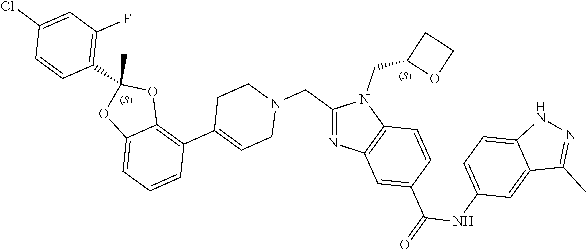 |
| 6 | 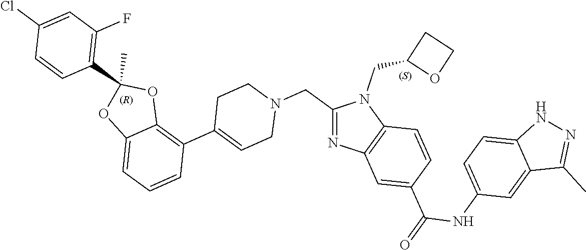 |
| 7 | 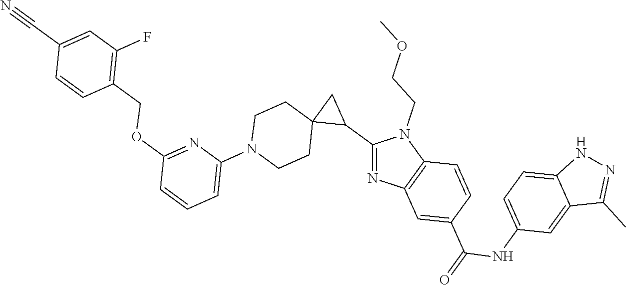 [SMILES: COCCn1c(C2CC23CCN(c2cccc(OCc4ccc(C#N)cc4F)n2)CC3)nc2cc(C(=O)Nc3ccc4[nH]nc(C)c4c3)ccc21] |
| 8 | 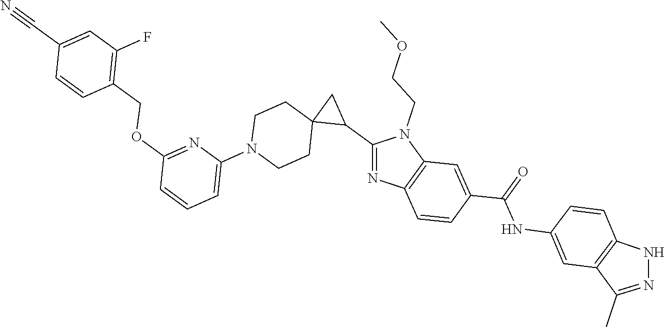 [SMILES: COCCn1c(C2CC23CCN(c2cccc(OCc4ccc(C#N)cc4F)n2)CC3)nc2ccc(C(=O)Nc3ccc4[nH]nc(C)c4c3)cc21] |
| 9 | 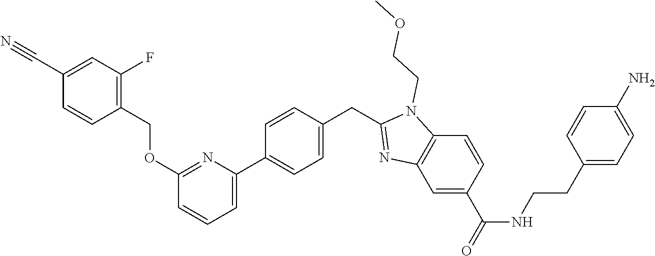 [SMILES: COCCn1c(Cc2ccc(-c3cccc(OCc4ccc(C#N)cc4F)n3)cc2)nc2cc(C(=O)NCCc3ccc(N)cc3)ccc21] |
| 10 | 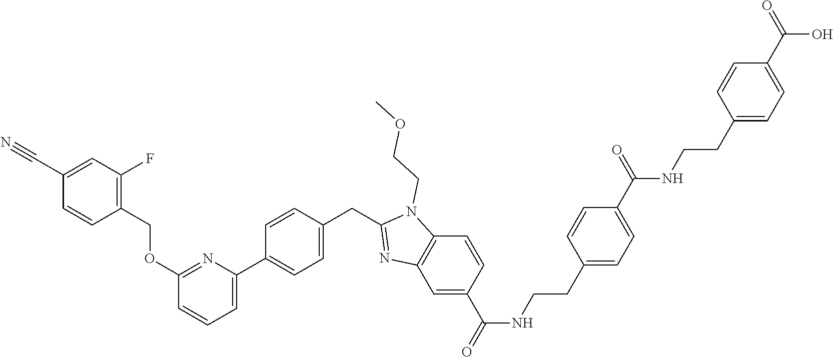 [SMILES: COCCn1c(Cc2ccc(-c3cccc(OCc4ccc(C#N)cc4F)n3)cc2)nc2cc(C(=O)NCCc3ccc(C(=O)NCCc4ccc(C(=O)O)cc4)cc3)ccc21] |
| 11 | 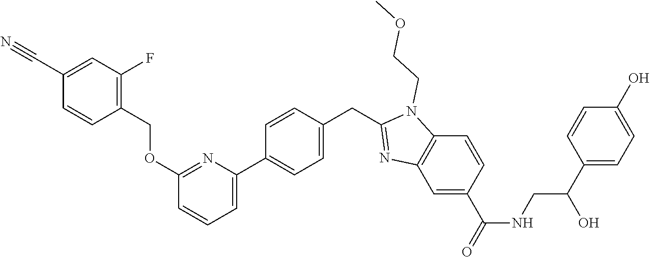 [SMILES: COCCn1c(Cc2ccc(-c3cccc(OCc4ccc(C#N)cc4F)n3)cc2)nc2cc(C(=O)NCC(O)c3ccc(O)cc3)ccc21] |
| 12 | 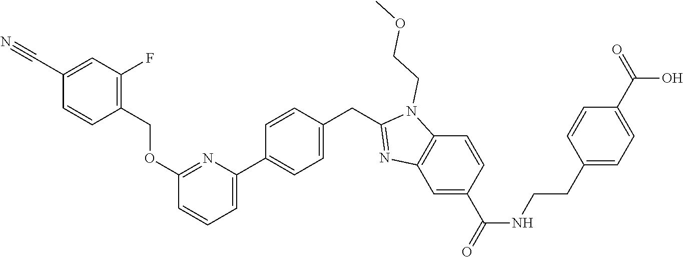 [SMILES: COCCn1c(Cc2ccc(-c3cccc(OCc4ccc(C#N)cc4F)n3)cc2)nc2cc(C(=O)NCCc3ccc(C(=O)O)cc3)ccc21] |
| 13 | 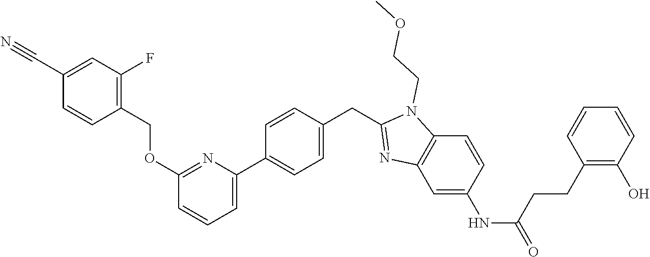 [SMILES: COCCn1c(Cc2ccc(-c3cccc(OCc4ccc(C#N)cc4F)n3)cc2)nc2cc(NC(=O)CCc3ccccc3O)ccc21] |
| 14 | 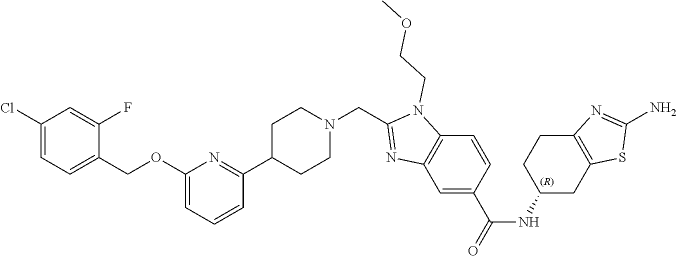 |
| 15 | 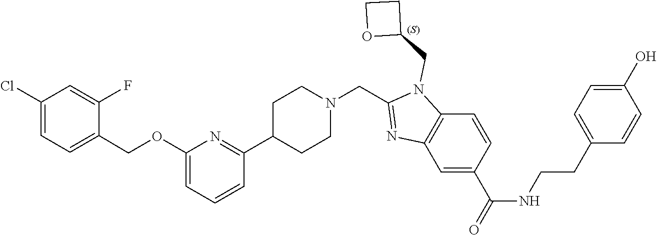 |
| 16 | 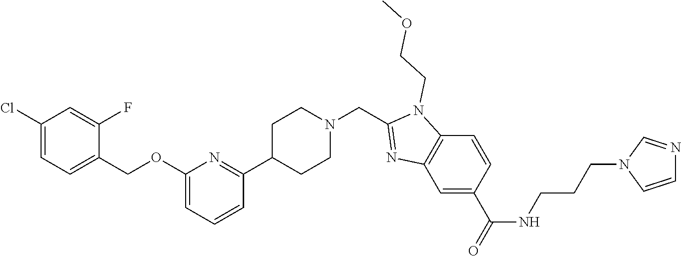 [SMILES: COCCn1c(CN2CCC(c3cccc(OCc4ccc(Cl)cc4F)n3)CC2)nc2cc(C(=O)NCCCn3ccnc3)ccc21] |
| 17 | 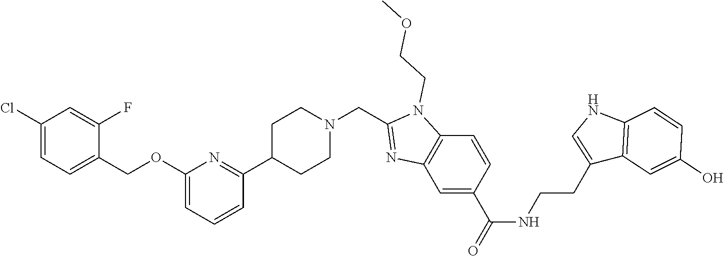 [SMILES: COCCn1c(CN2CCC(c3cccc(OCc4ccc(Cl)cc4F)n3)CC2)nc2cc(C(=O)NCCc3c[nH]c4ccc(O)cc34)ccc21] |
| 18 | 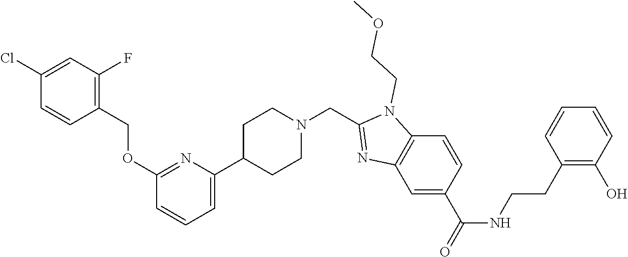 [SMILES: COCCn1c(CN2CCC(c3cccc(OCc4ccc(Cl)cc4F)n3)CC2)nc2cc(C(=O)NCCc3ccccc3O)ccc21] |
| 19 | 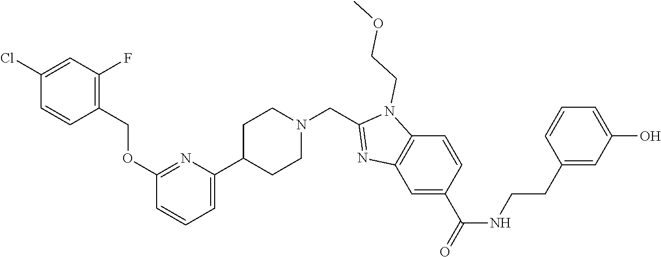 [SMILES: COCCn1c(CN2CCC(c3cccc(OCc4ccc(Cl)cc4F)n3)CC2)nc2cc(C(=O)NCCc3cccc(O)c3)ccc21] |
| 20 | 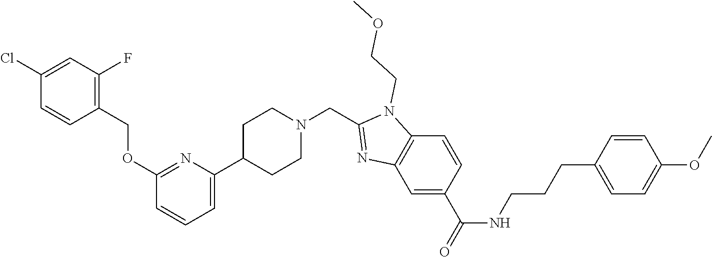 [SMILES: COCCn1c(CN2CCC(c3cccc(OCc4ccc(Cl)cc4F)n3)CC2)nc2cc(C(=O)NCCCc3ccc(OC)cc3)ccc21] |
| 21 | 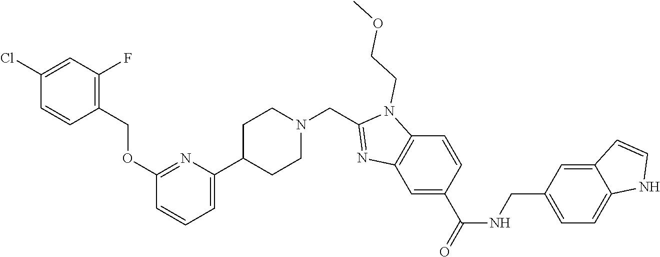 [SMILES: COCCn1c(CN2CCC(c3cccc(OCc4ccc(Cl)cc4F)n3)CC2)nc2cc(C(=O)NCc3ccc4[nH]ccc4c3)ccc21] |
| 22 | 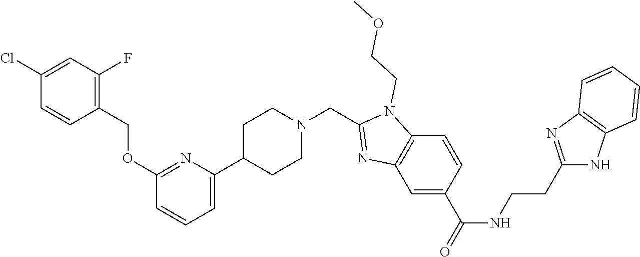 [SMILES: COCCn1c(CN2CCC(c3cccc(OCc4ccc(Cl)cc4F)n3)CC2)nc2cc(C(=O)NCCc3nc4ccccc4[nH]3)ccc21] |
| 23 | 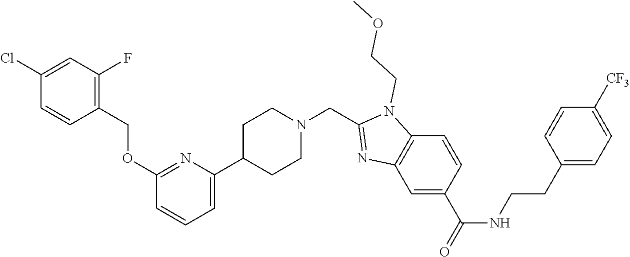 [SMILES: COCCn1c(CN2CCC(c3cccc(OCc4ccc(Cl)cc4F)n3)CC2)nc2cc(C(=O)NCCc3ccc(C(F)(F)F)cc3)ccc21] |
| 24 | 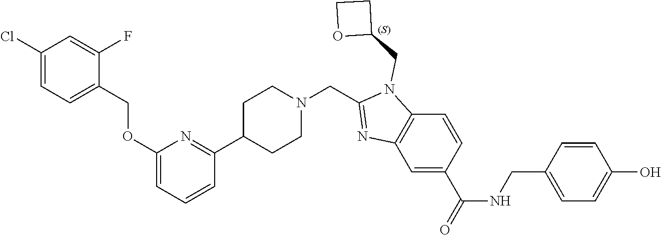 |
| 25 | 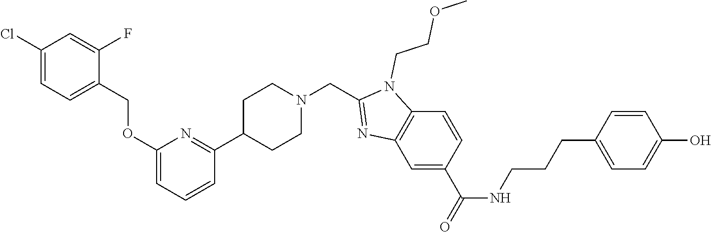 [SMILES: COCCn1c(CN2CCC(c3cccc(OCc4ccc(Cl)cc4F)n3)CC2)nc2cc(C(=O)NCCCc3ccc(O)cc3)ccc21] |
| 26 | 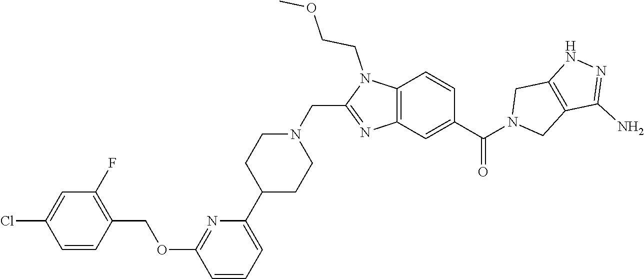 [SMILES: COCCn1c(CN2CCC(c3cccc(OCc4ccc(Cl)cc4F)n3)CC2)nc2cc(C(=O)N3Cc4[nH]nc(N)c4C3)ccc21] |
| 27 | 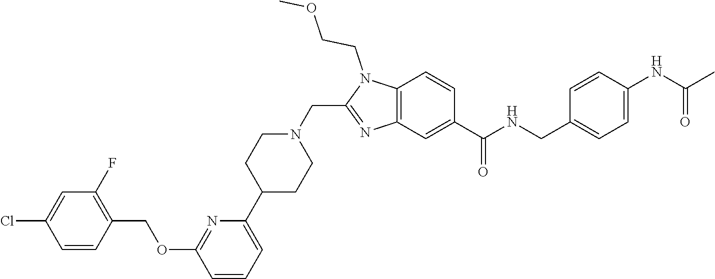 [SMILES: COCCn1c(CN2CCC(c3cccc(OCc4ccc(Cl)cc4F)n3)CC2)nc2cc(C(=O)NCc3ccc(NC(C)=O)cc3)ccc21] |
| 28 | 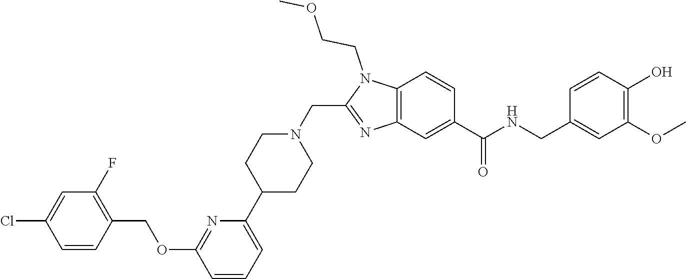 [SMILES: COCCn1c(CN2CCC(c3cccc(OCc4ccc(Cl)cc4F)n3)CC2)nc2cc(C(=O)NCc3ccc(O)c(OC)c3)ccc21] |
| 29 | 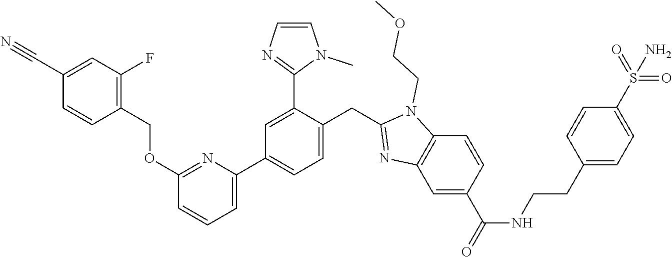 [SMILES: COCCn1c(Cc2ccc(-c3cccc(OCc4ccc(C#N)cc4F)n3)cc2-c2nccn2C)nc2cc(C(=O)NCCc3ccc(S(N)(=O)=O)cc3)ccc21] |
| 30 | 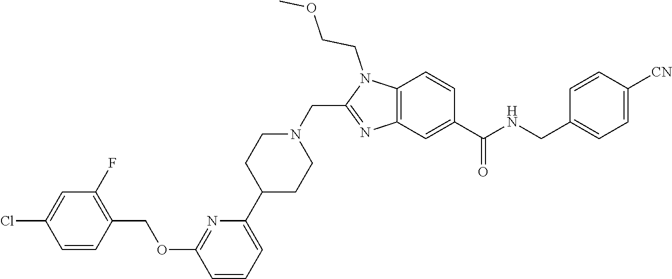 [SMILES: COCCn1c(CN2CCC(c3cccc(OCc4ccc(Cl)cc4F)n3)CC2)nc2cc(C(=O)NCc3ccc(C#N)cc3)ccc21] |
| 31 | 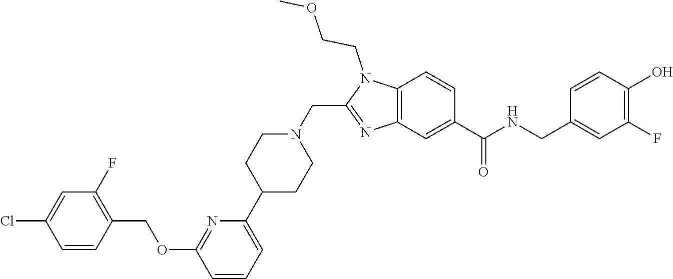 [SMILES: COCCn1c(CN2CCC(c3cccc(OCc4ccc(Cl)cc4F)n3)CC2)nc2cc(C(=O)NCc3ccc(O)c(F)c3)ccc21] |
| 32 | 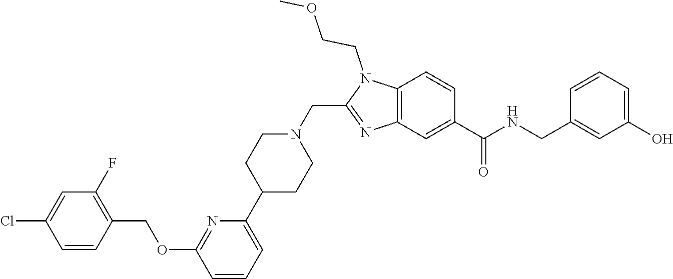 [SMILES: COCCn1c(CN2CCC(c3cccc(OCc4ccc(Cl)cc4F)n3)CC2)nc2cc(C(=O)NCc3cccc(O)c3)ccc21] |
| 33 | 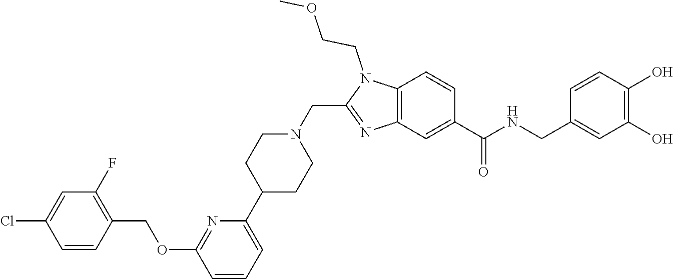 [SMILES: COCCn1c(CN2CCC(c3cccc(OCc4ccc(Cl)cc4F)n3)CC2)nc2cc(C(=O)NCc3ccc(O)c(O)c3)ccc21] |
| 34 | 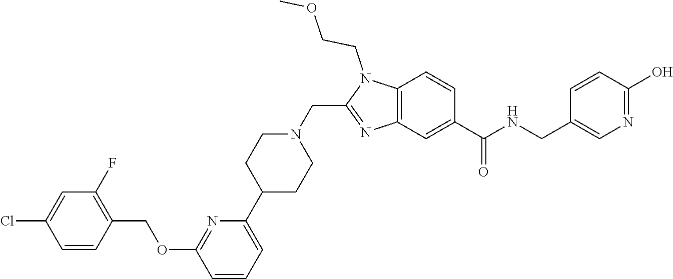 [SMILES: COCCn1c(CN2CCC(c3cccc(OCc4ccc(Cl)cc4F)n3)CC2)nc2cc(C(=O)NCc3ccc(O)nc3)ccc21] |
| 35 | 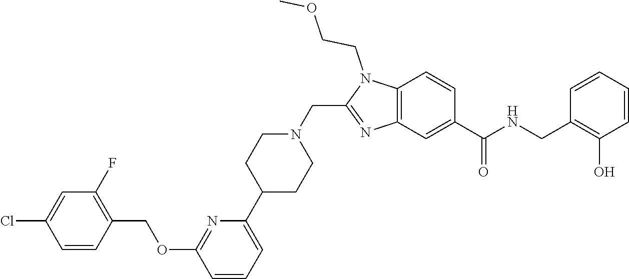 [SMILES: COCCn1c(CN2CCC(c3cccc(OCc4ccc(Cl)cc4F)n3)CC2)nc2cc(C(=O)NCc3ccccc3O)ccc21] |
| 36 | 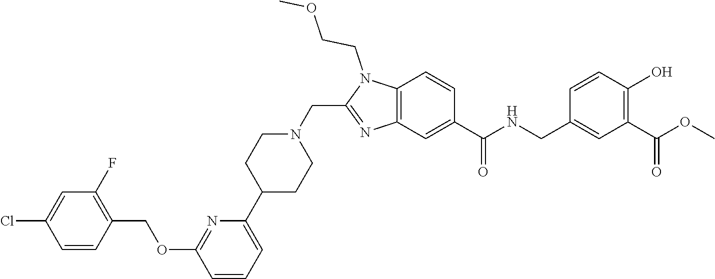 [SMILES: COCCn1c(CN2CCC(c3cccc(OCc4ccc(Cl)cc4F)n3)CC2)nc2cc(C(=O)NCc3ccc(O)c(C(=O)OC)c3)ccc21] |
| 37 | 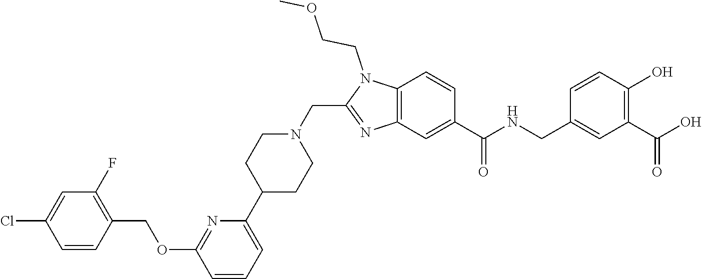 [SMILES: COCCn1c(CN2CCC(c3cccc(OCc4ccc(Cl)cc4F)n3)CC2)nc2cc(C(=O)NCc3ccc(O)c(C(=O)O)c3)ccc21] |
| 38 | 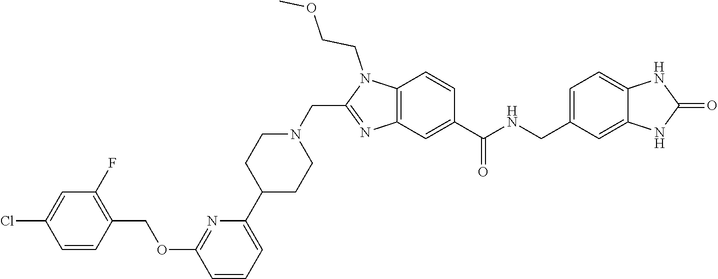 [SMILES: COCCn1c(CN2CCC(c3cccc(OCc4ccc(Cl)cc4F)n3)CC2)nc2cc(C(=O)NCc3ccc4[nH]c(=O)[nH]c4c3)ccc21] |
| 39 | 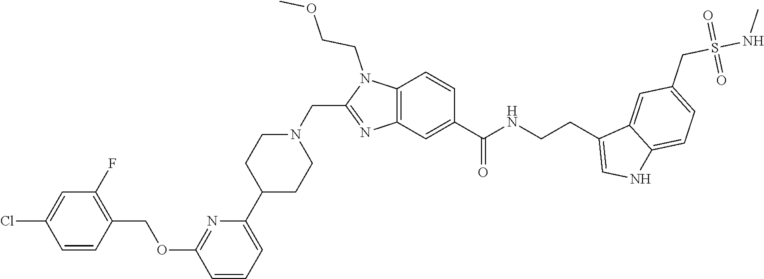 [SMILES: CNS(=O)(=O)Cc1ccc2[nH]cc(CCNC(=O)c3ccc4c(c3)nc(CN3CCC(c5cccc(OCc6ccc(Cl)cc6F)n5)CC3)n4CCOC)c2c1] |
| 40 | 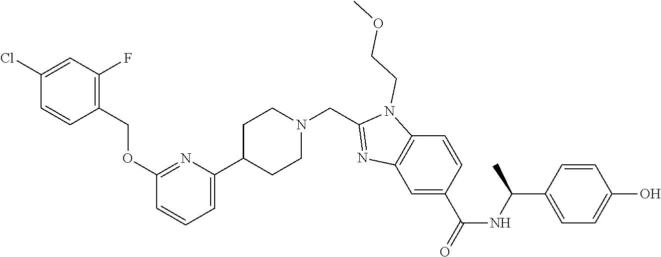 [SMILES: COCCn1c(CN2CCC(c3cccc(OCc4ccc(Cl)cc4F)n3)CC2)nc2cc(C(=O)N[C@@H](C)c3ccc(O)cc3)ccc21] |
| 41 | 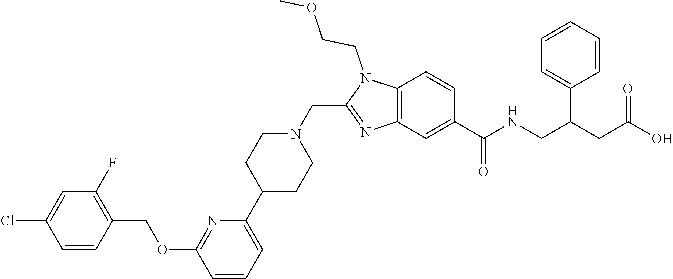 [SMILES: COCCn1c(CN2CCC(c3cccc(OCc4ccc(Cl)cc4F)n3)CC2)nc2cc(C(=O)NCC(CC(=O)O)c3ccccc3)ccc21] |
| 42 | 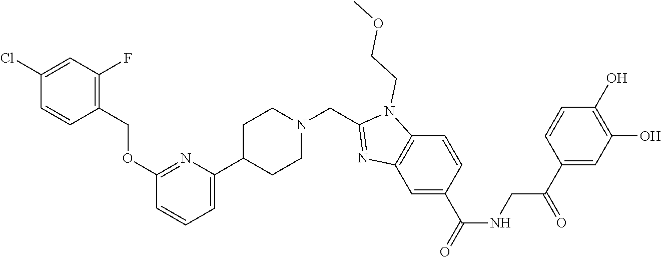 [SMILES: COCCn1c(CN2CCC(c3cccc(OCc4ccc(Cl)cc4F)n3)CC2)nc2cc(C(=O)NCC(=O)c3ccc(O)c(O)c3)ccc21] |
| 43 | 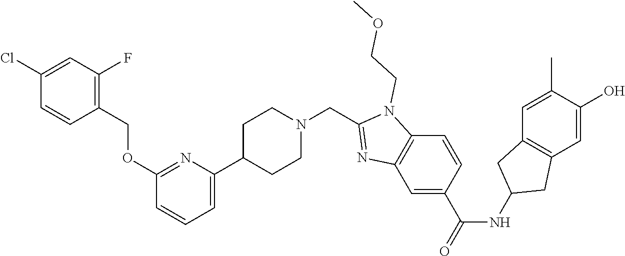 [SMILES: COCCn1c(CN2CCC(c3cccc(OCc4ccc(Cl)cc4F)n3)CC2)nc2cc(C(=O)NC3Cc4cc(C)c(O)cc4C3)ccc21] |
| 44 | 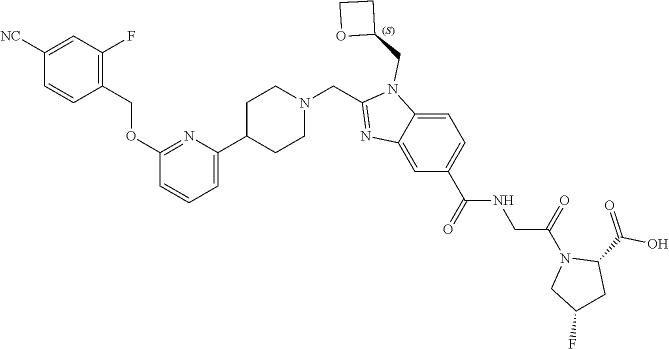 |
| 45 | 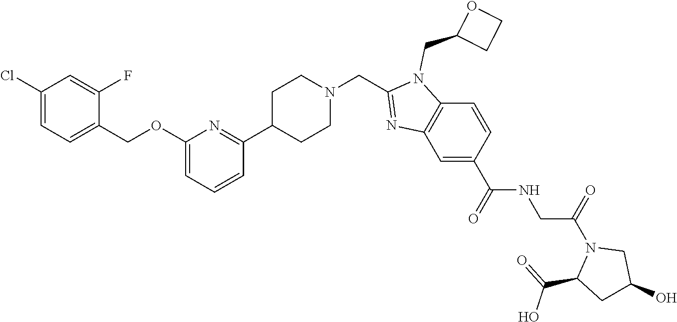 [SMILES: O=C(NCC(=O)N1C[C@@H](O)C[C@H]1C(=O)O)c1ccc2c(c1)nc(CN1CCC(c3cccc(OCc4ccc(Cl)cc4F)n3)CC1)n2C[C@@H]1CCO1] |
| 46 | 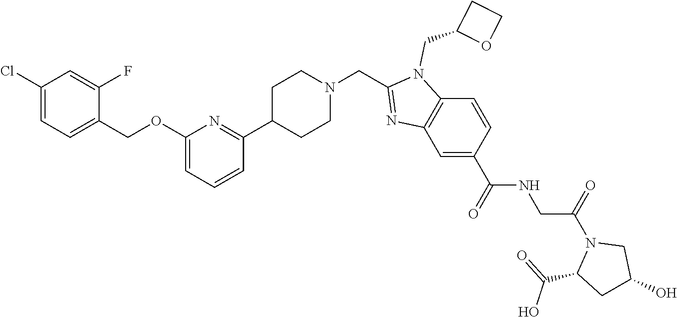 [SMILES: O=C(NCC(=O)N1C[C@H](O)C[C@@H]1C(=O)O)c1ccc2c(c1)nc(CN1CCC(c3cccc(OCc4ccc(Cl)cc4F)n3)CC1)n2C[C@@H]1CCO1] |
| 47 | 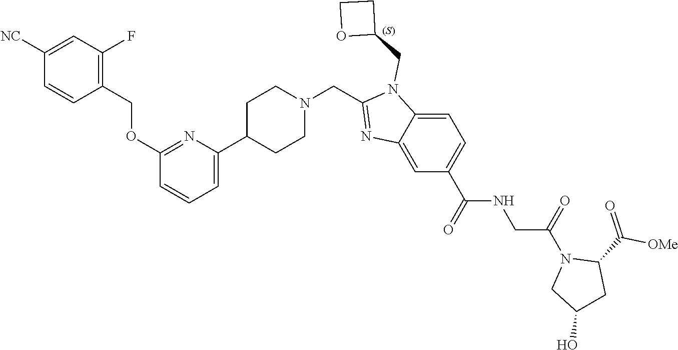 |
| 48 | 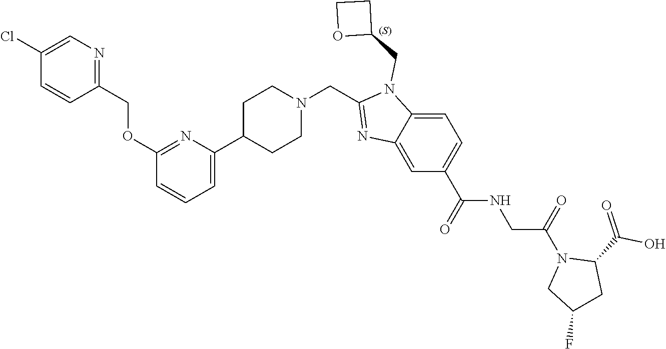 |
| 49 | 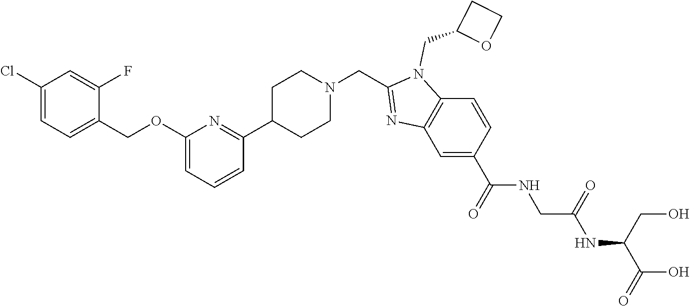 [SMILES: O=C(CNC(=O)c1ccc2c(c1)nc(CN1CCC(c3cccc(OCc4ccc(Cl)cc4F)n3)CC1)n2C[C@@H]1CCO1)N[C@@H](CO)C(=O)O] |
| 50 | 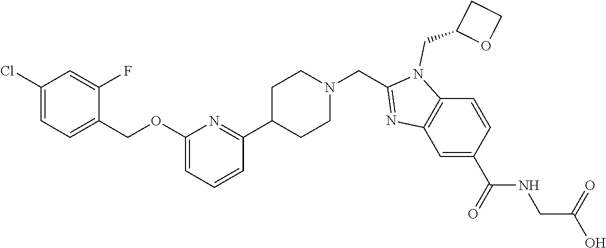 [SMILES: O=C(O)CNC(=O)c1ccc2c(c1)nc(CN1CCC(c3cccc(OCc4ccc(Cl)cc4F)n3)CC1)n2C[C@@H]1CCO1] |
| 51 | 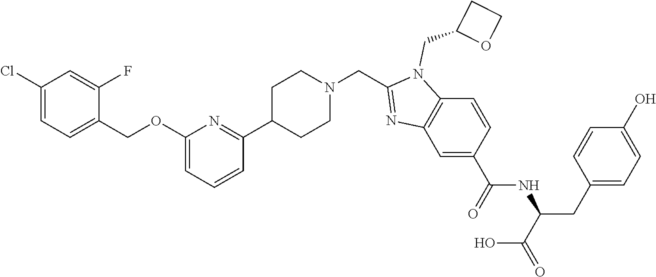 [SMILES: O=C(N[C@@H](Cc1ccc(O)cc1)C(=O)O)c1ccc2c(c1)nc(CN1CCC(c3cccc(OCc4ccc(Cl)cc4F)n3)CC1)n2C[C@@H]1CCO1] |
| 52 | 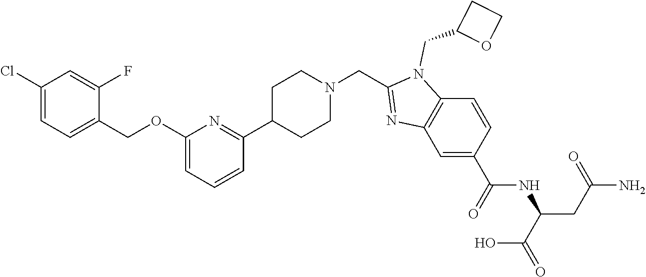 |
| 53 | 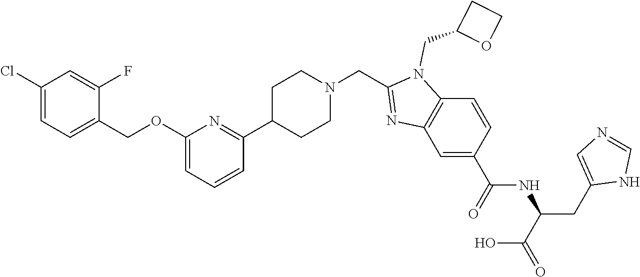 [SMILES: O=C(N[C@@H](Cc1cnc[nH]1)C(=O)O)c1ccc2c(c1)nc(CN1CCC(c3cccc(OCc4ccc(Cl)cc4F)n3)CC1)n2C[C@@H]1CCO1] |
| 54 | 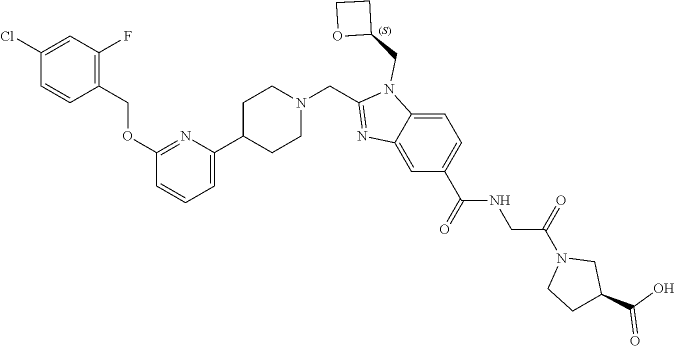 |
| 55 | 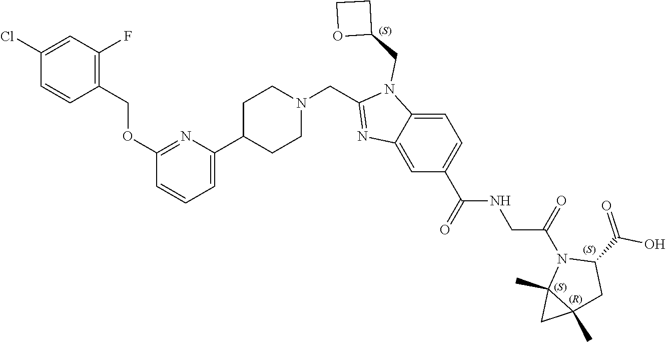 |
| 56 | 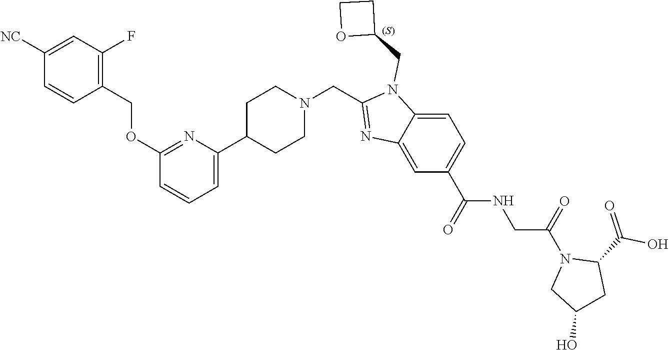 |
| 57 | 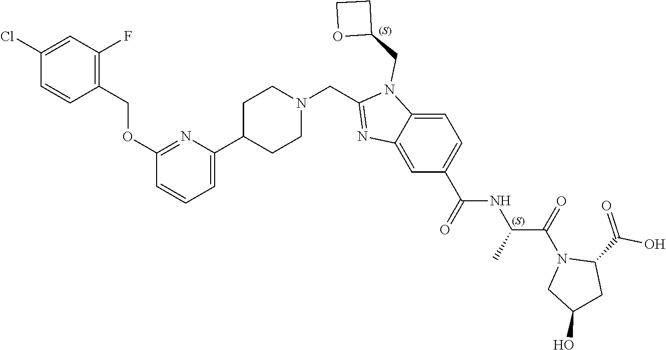 |
| 58 | 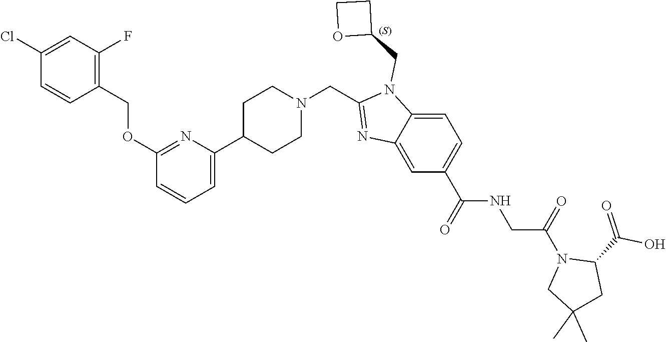 |
| 59 | 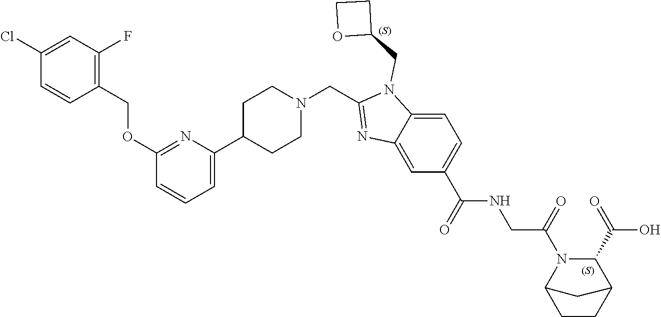 |
| 60 | 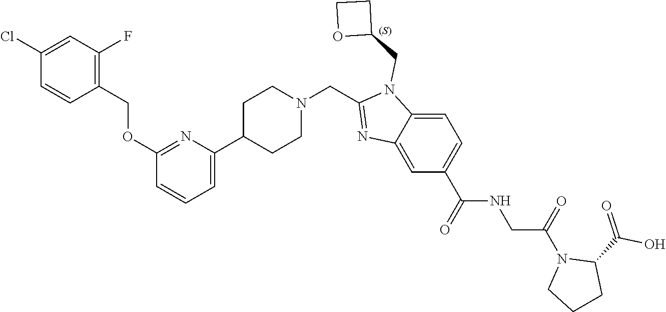 |
| 61 | 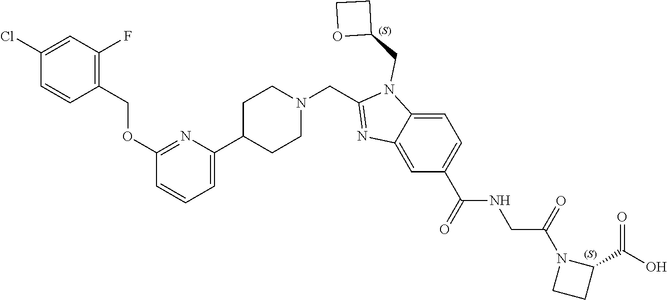 |
| 62 | 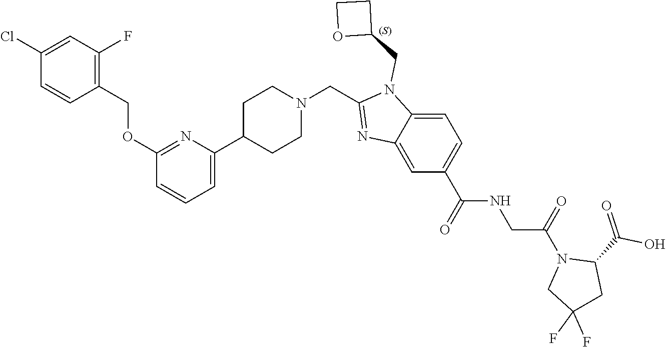 |
| 63 | 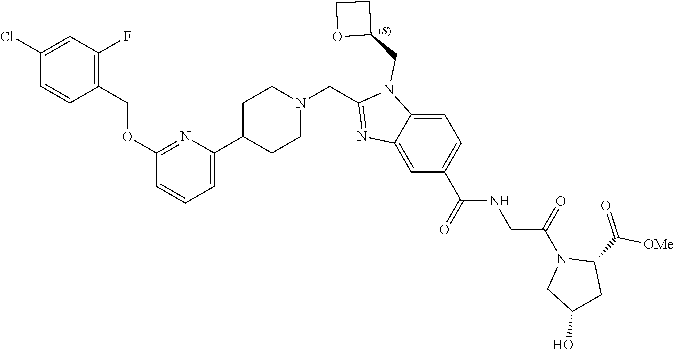 |
| 64 | 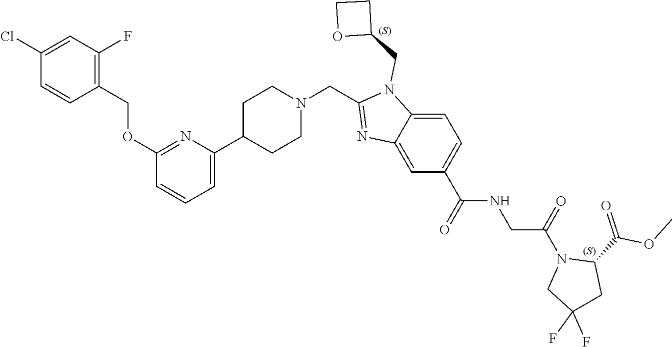 |
| 65 | 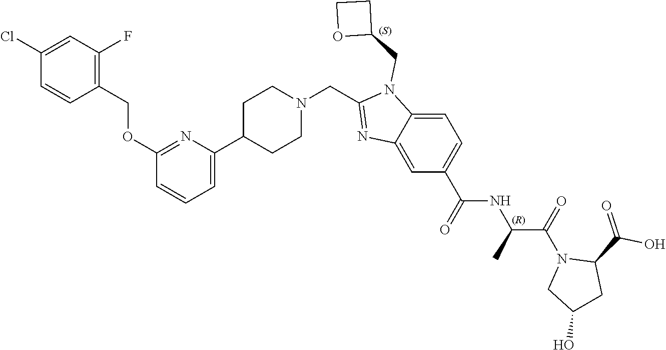 |
| 66 | 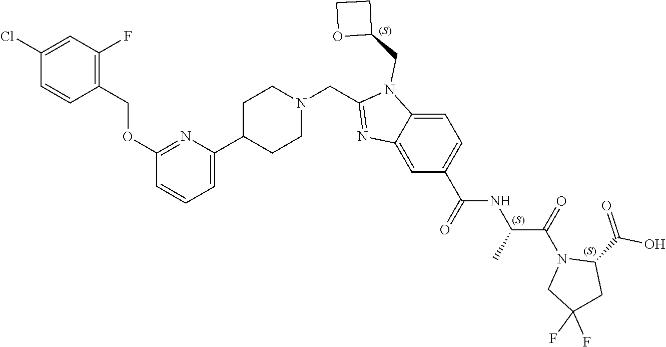 |
| 67 | 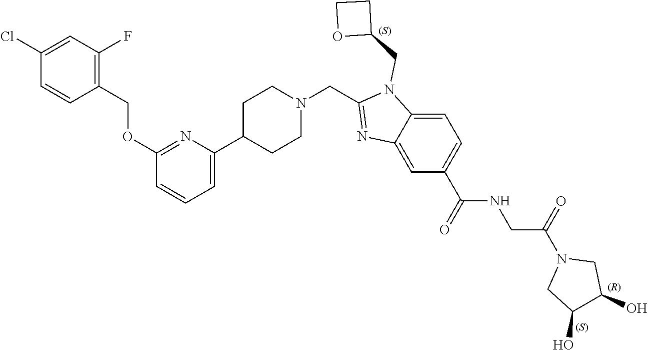 |
| 68 | 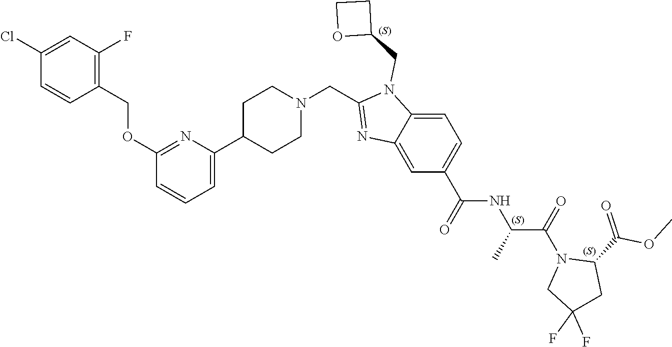 |
| 69 | 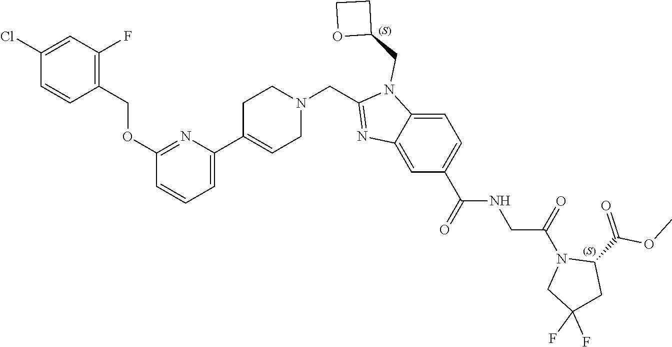 |
| 70 | 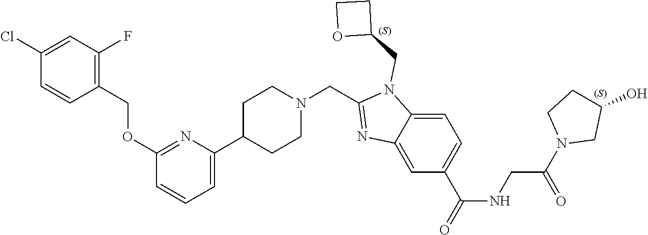 |
| 71 | 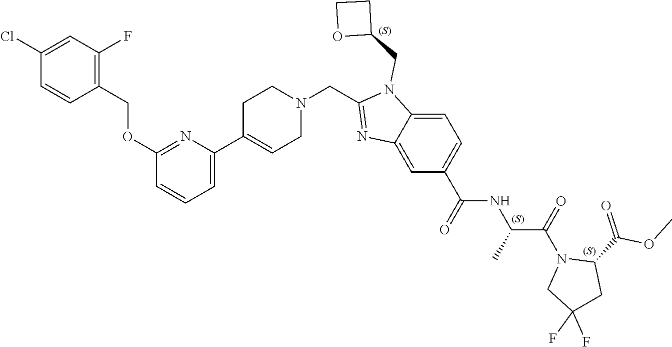 |
| 72 | 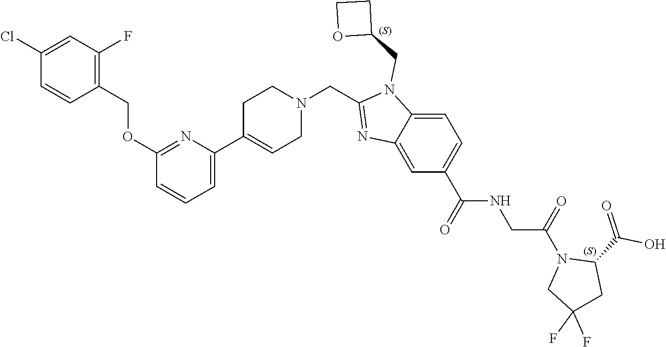 |
| 73 | 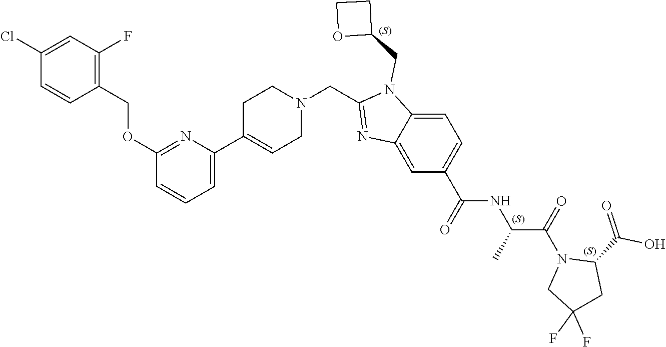 |
| 74 | 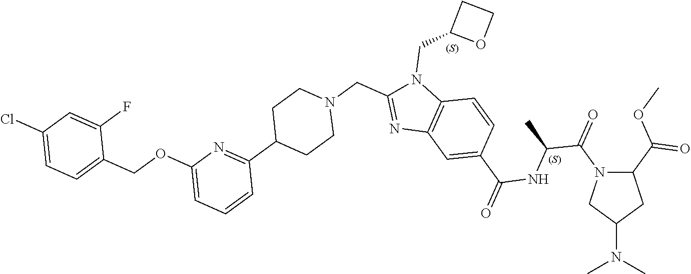 |
| 75 | 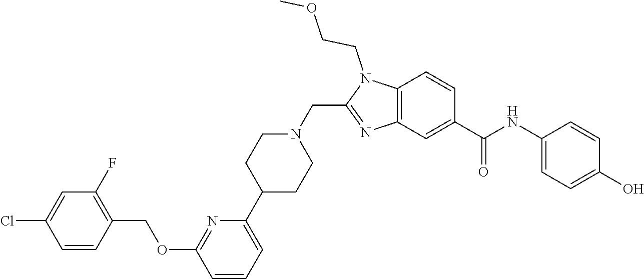 [SMILES: COCCn1c(CN2CCC(c3cccc(OCc4ccc(Cl)cc4F)n3)CC2)nc2cc(C(=O)Nc3ccc(O)cc3)ccc21] |
| 76 | 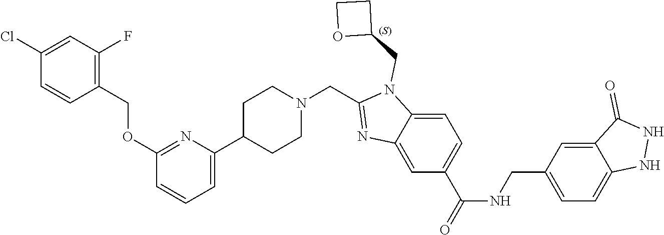 |
| 77 | 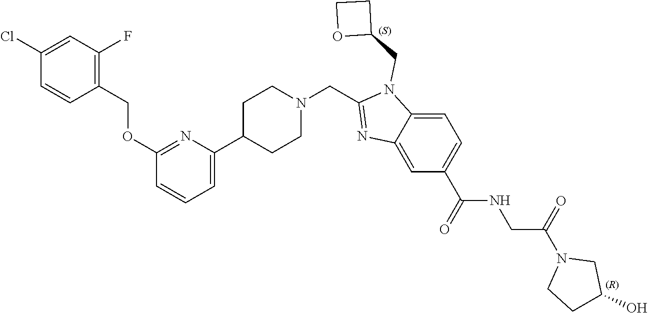 |
| 78 | 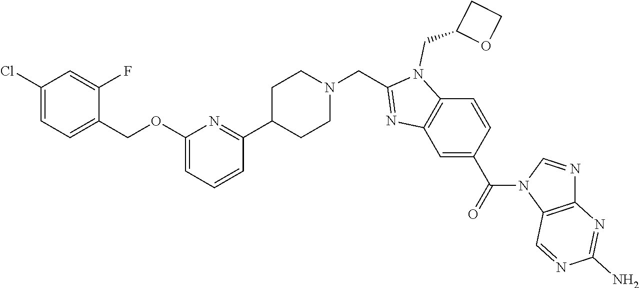 [SMILES: Nc1ncc2c(ncn2C(=O)c2ccc3c(c2)nc(CN2CCC(c4cccc(OCc5ccc(Cl)cc5F)n4)CC2)n3C[C@@H]2CCO2)n1] |
| 79 | 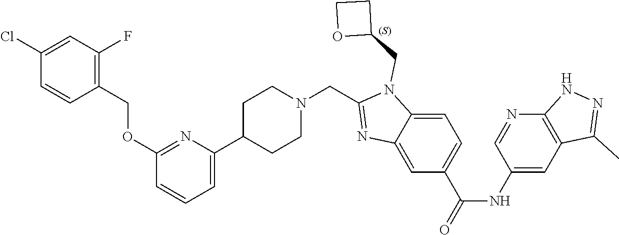 |
| 80 | 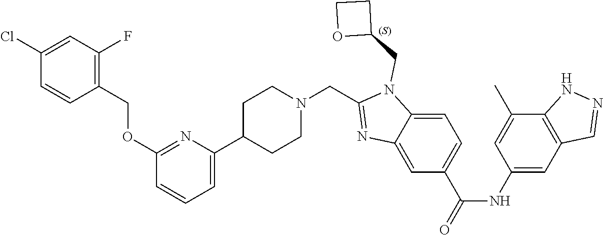 |
| 81 | 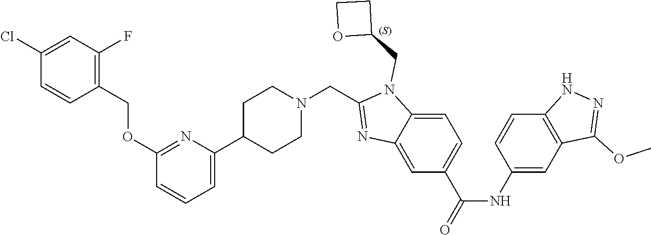 |
| 82 | 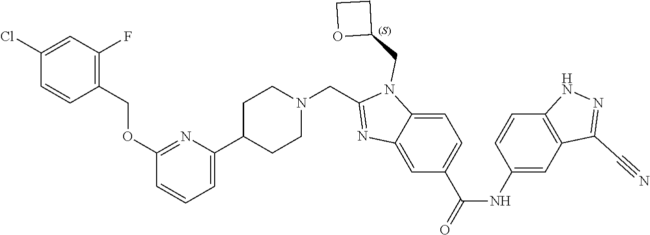 |
| 83 | 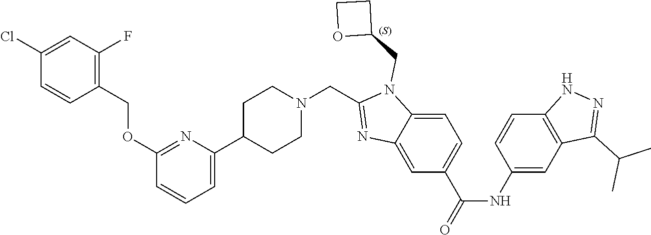 |
| 84 | 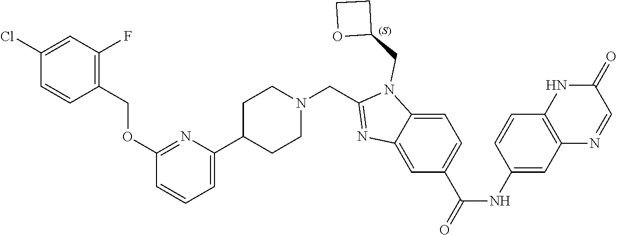 |
| 85 | 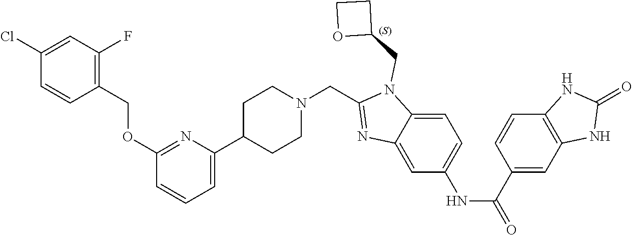 |
| 86 | 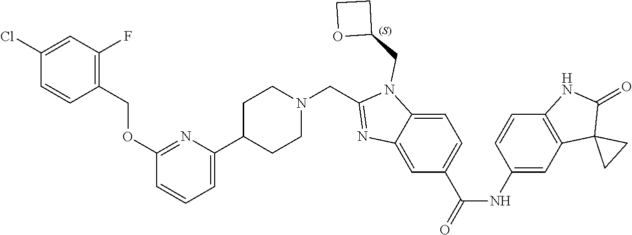 |
| 87 | 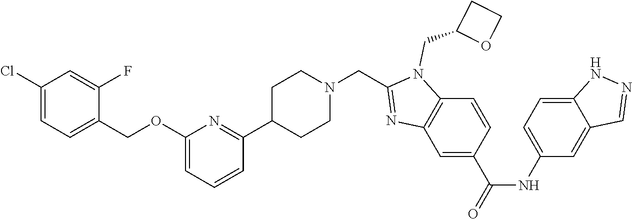 [SMILES: O=C(Nc1ccc2[nH]ncc2c1)c1ccc2c(c1)nc(CN1CCC(c3cccc(OCc4ccc(Cl)cc4F)n3)CC1)n2C[C@@H]1CCO1] |
| 88 | 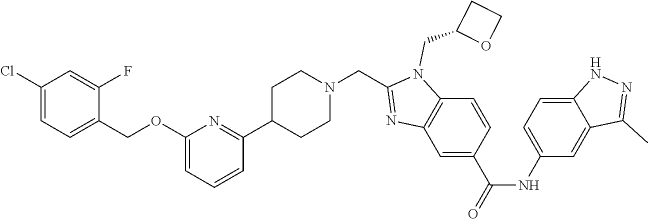 [SMILES: Cc1n[nH]c2ccc(NC(=O)c3ccc4c(c3)nc(CN3CCC(c5cccc(OCc6ccc(Cl)cc6F)n5)CC3)n4C[C@@H]3CCO3)cc12] |
| 89 | 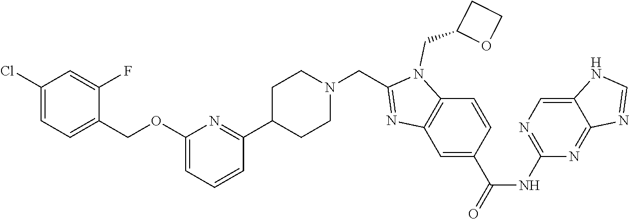 [SMILES: O=C(Nc1ncc2[nH]cnc2n1)c1ccc2c(c1)nc(CN1CCC(c3cccc(OCc4ccc(Cl)cc4F)n3)CC1)n2C[C@@H]1CCO1] |
| 90 | 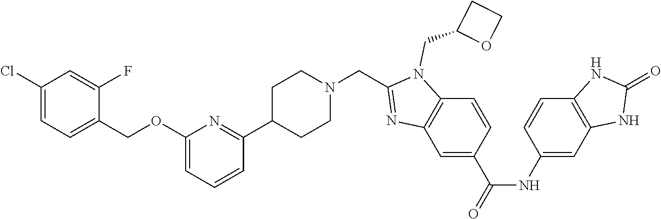 [SMILES: O=C(Nc1ccc2[nH]c(=O)[nH]c2c1)c1ccc2c(c1)nc(CN1CCC(c3cccc(OCc4ccc(Cl)cc4F)n3)CC1)n2C[C@@H]1CCO1] |
| 91 | 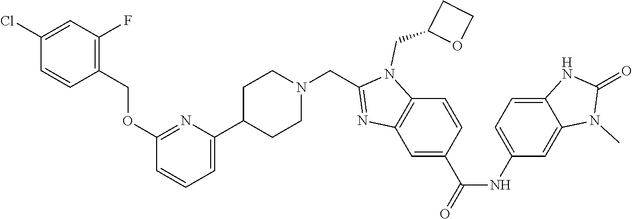 [SMILES: Cn1c(=O)[nH]c2ccc(NC(=O)c3ccc4c(c3)nc(CN3CCC(c5cccc(OCc6ccc(Cl)cc6F)n5)CC3)n4C[C@@H]3CCO3)cc21] |
| 92 | 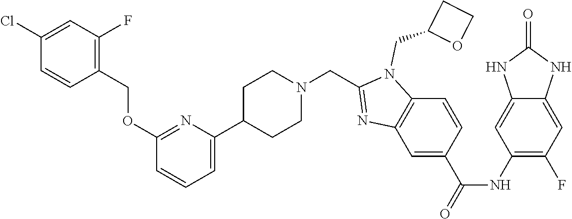 [SMILES: O=C(Nc1cc2[nH]c(=O)[nH]c2cc1F)c1ccc2c(c1)nc(CN1CCC(c3cccc(OCc4ccc(Cl)cc4F)n3)CC1)n2C[C@@H]1CCO1] |
| 93 | 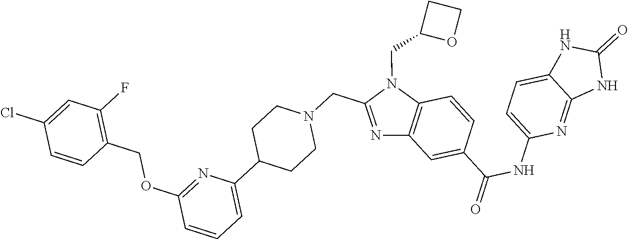 [SMILES: O=C(Nc1ccc2[nH]c(=O)[nH]c2n1)c1ccc2c(c1)nc(CN1CCC(c3cccc(OCc4ccc(Cl)cc4F)n3)CC1)n2C[C@@H]1CCO1] |
| 94 | 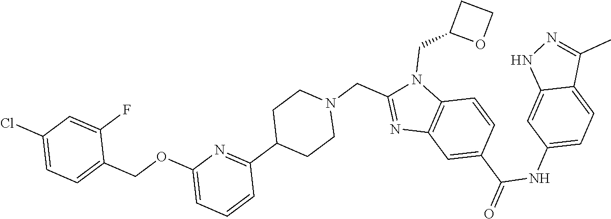 [SMILES: Cc1n[nH]c2cc(NC(=O)c3ccc4c(c3)nc(CN3CCC(c5cccc(OCc6ccc(Cl)cc6F)n5)CC3)n4C[C@@H]3CCO3)ccc12] |
| 95 | 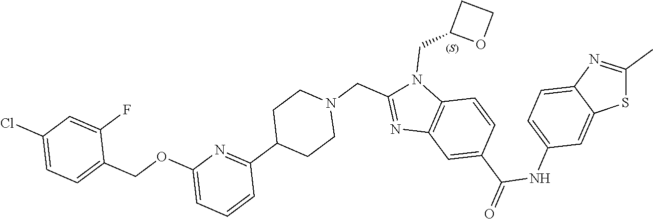 |
| 96 | 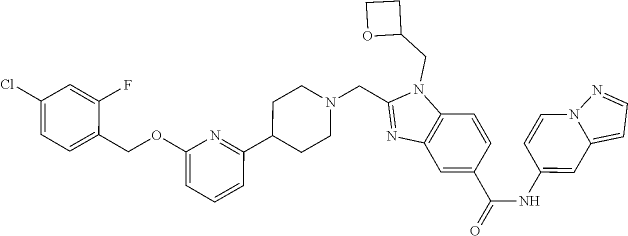 [SMILES: O=C(Nc1ccn2nccc2c1)c1ccc2c(c1)nc(CN1CCC(c3cccc(OCc4ccc(Cl)cc4F)n3)CC1)n2CC1CCO1] |
| 97 | 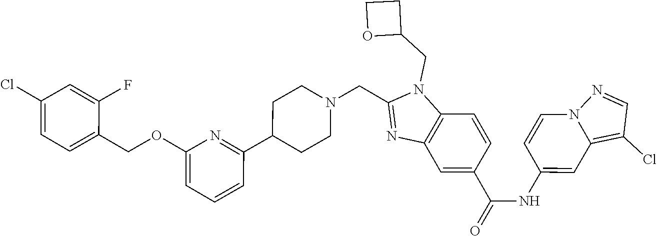 [SMILES: O=C(Nc1ccn2ncc(Cl)c2c1)c1ccc2c(c1)nc(CN1CCC(c3cccc(OCc4ccc(Cl)cc4F)n3)CC1)n2CC1CCO1] |
| 98 | 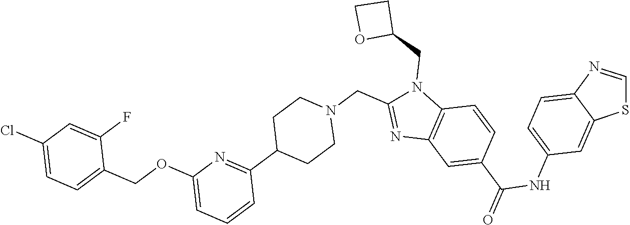 [SMILES: O=C(Nc1ccc2ncsc2c1)c1ccc2c(c1)nc(CN1CCC(c3cccc(OCc4ccc(Cl)cc4F)n3)CC1)n2C[C@@H]1CCO1] |
| 99 | 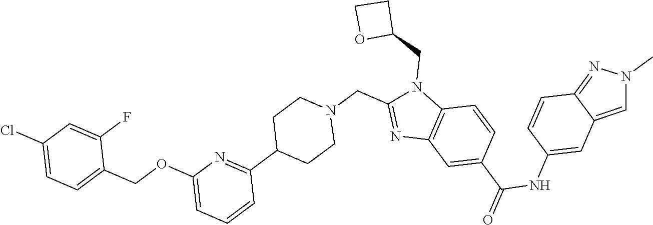 [SMILES: Cn1cc2cc(NC(=O)c3ccc4c(c3)nc(CN3CCC(c5cccc(OCc6ccc(Cl)cc6F)n5)CC3)n4C[C@@H]3CCO3)ccc2n1] |
| 100 | 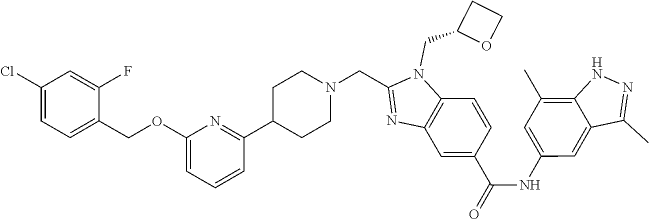 [SMILES: Cc1n[nH]c2c(C)cc(NC(=O)c3ccc4c(c3)nc(CN3CCC(c5cccc(OCc6ccc(Cl)cc6F)n5)CC3)n4C[C@@H]3CCO3)cc12] |
| 101 | 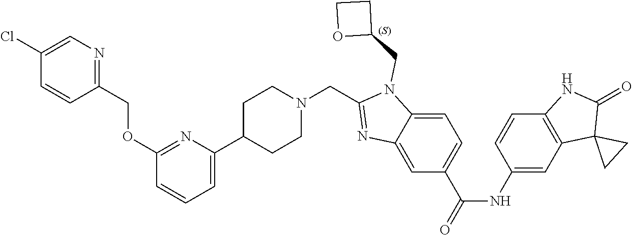 |
| 102 | 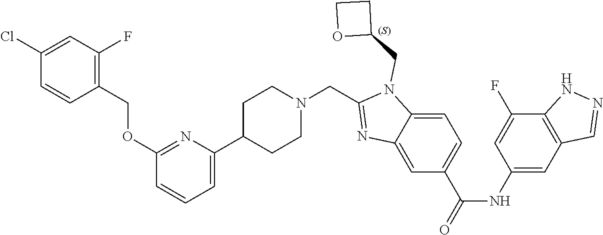 |
| 103 | 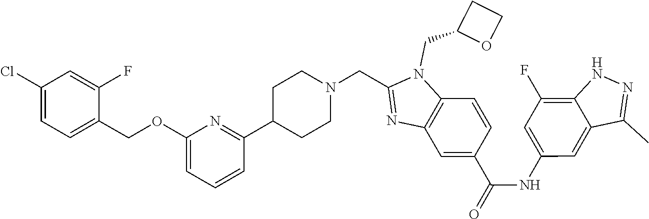 [SMILES: Cc1n[nH]c2c(F)cc(NC(=O)c3ccc4c(c3)nc(CN3CCC(c5cccc(OCc6ccc(Cl)cc6F)n5)CC3)n4C[C@@H]3CCO3)cc12] |
| 104 | 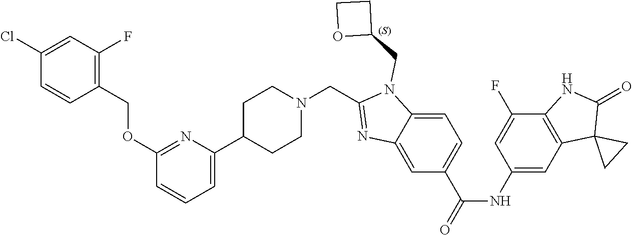 |
| 105 | 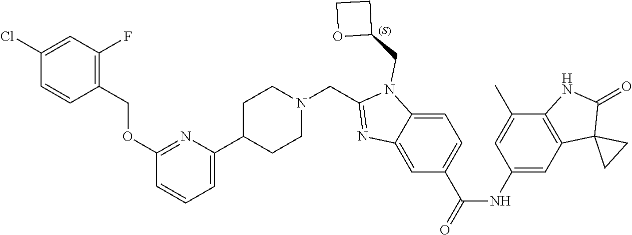 |
| 106 | 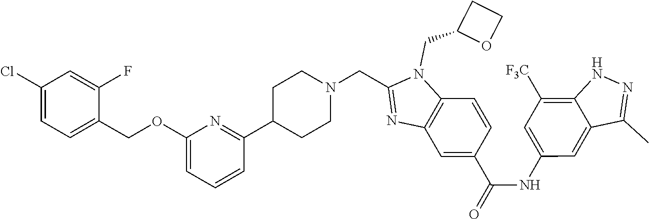 [SMILES: Cc1n[nH]c2c(C(F)(F)F)cc(NC(=O)c3ccc4c(c3)nc(CN3CCC(c5cccc(OCc6ccc(Cl)cc6F)n5)CC3)n4C[C@@H]3CCO3)cc12] |
| 107 | 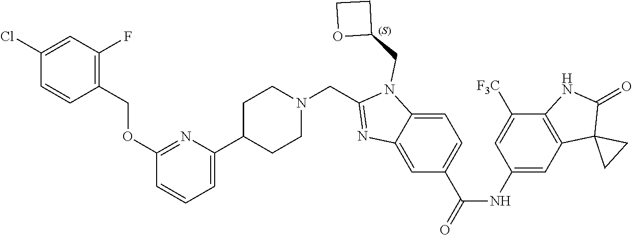 |
| 108 | 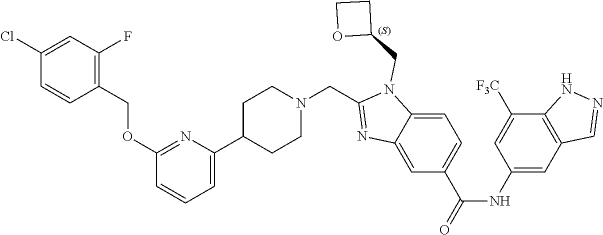 |
| 109 | 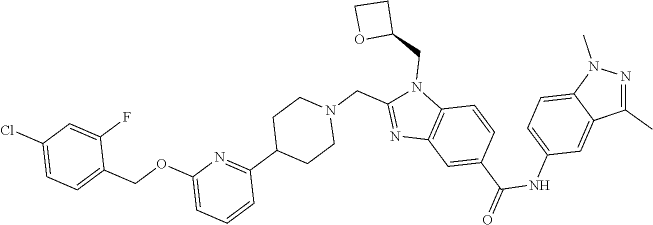 [SMILES: Cc1nn(C)c2ccc(NC(=O)c3ccc4c(c3)nc(CN3CCC(c5cccc(OCc6ccc(Cl)cc6F)n5)CC3)n4C[C@@H]3CCO3)cc12] |
| 110 | 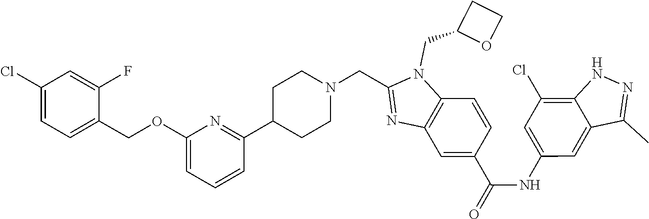 [SMILES: Cc1n[nH]c2c(Cl)cc(NC(=O)c3ccc4c(c3)nc(CN3CCC(c5cccc(OCc6ccc(Cl)cc6F)n5)CC3)n4C[C@@H]3CCO3)cc12] |
| 111 | 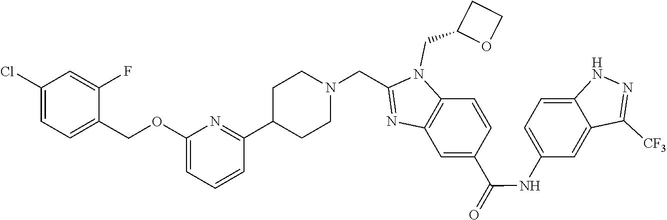 [SMILES: O=C(Nc1ccc2[nH]nc(C(F)(F)F)c2c1)c1ccc2c(c1)nc(CN1CCC(c3cccc(OCc4ccc(Cl)cc4F)n3)CC1)n2C[C@@H]1CCO1] |
| 112 | 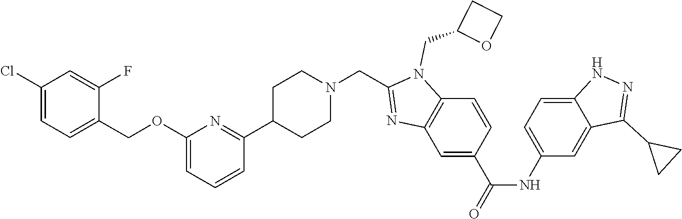 [SMILES: O=C(Nc1ccc2[nH]nc(C3CC3)c2c1)c1ccc2c(c1)nc(CN1CCC(c3cccc(OCc4ccc(Cl)cc4F)n3)CC1)n2C[C@@H]1CCO1] |
| 113 | 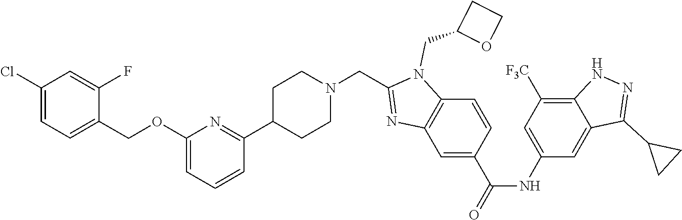 [SMILES: O=C(Nc1cc(C(F)(F)F)c2[nH]nc(C3CC3)c2c1)c1ccc2c(c1)nc(CN1CCC(c3cccc(OCc4ccc(Cl)cc4F)n3)CC1)n2C[C@@H]1CCO1] |
| 114 | 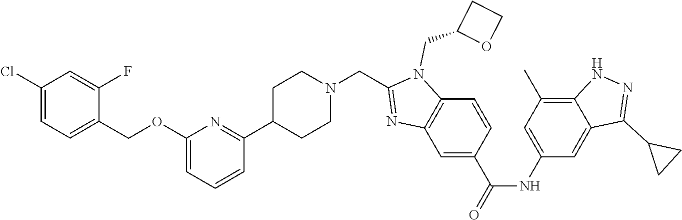 [SMILES: Cc1cc(NC(=O)c2ccc3c(c2)nc(CN2CCC(c4cccc(OCc5ccc(Cl)cc5F)n4)CC2)n3C[C@@H]2CCO2)cc2c(C3CC3)n[nH]c12] |
| 115 | 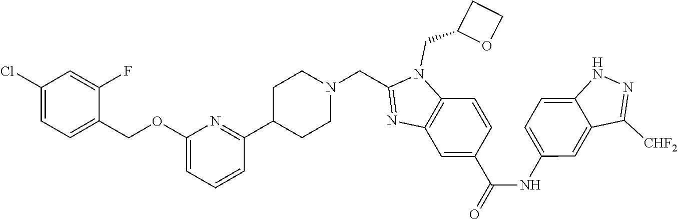 [SMILES: O=C(Nc1ccc2[nH]nc(C(F)F)c2c1)c1ccc2c(c1)nc(CN1CCC(c3cccc(OCc4ccc(Cl)cc4F)n3)CC1)n2C[C@@H]1CCO1] |
| 116 | 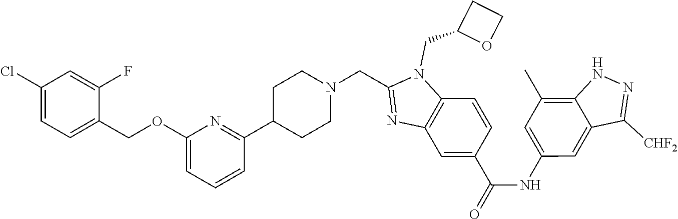 [SMILES: Cc1cc(NC(=O)c2ccc3c(c2)nc(CN2CCC(c4cccc(OCc5ccc(Cl)cc5F)n4)CC2)n3C[C@@H]2CCO2)cc2c(C(F)F)n[nH]c12] |
| 117 | 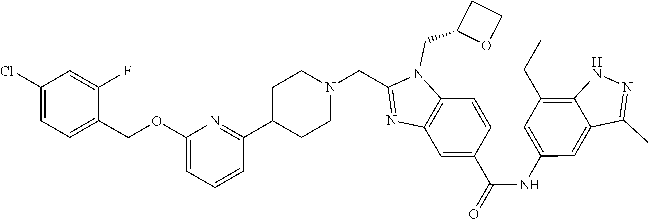 [SMILES: CCc1cc(NC(=O)c2ccc3c(c2)nc(CN2CCC(c4cccc(OCc5ccc(Cl)cc5F)n4)CC2)n3C[C@@H]2CCO2)cc2c(C)n[nH]c12] |
| 118 | 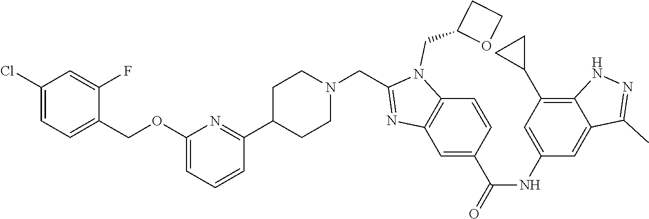 [SMILES: Cc1n[nH]c2c(C3CC3)cc(NC(=O)c3ccc4c(c3)nc(CN3CCC(c5cccc(OCc6ccc(Cl)cc6F)n5)CC3)n4C[C@@H]3CCO3)cc12] |
| 119 | 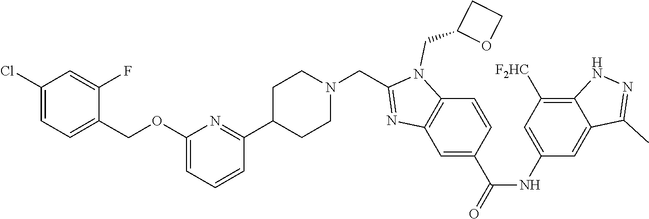 [SMILES: Cc1n[nH]c2c(C(F)F)cc(NC(=O)c3ccc4c(c3)nc(CN3CCC(c5cccc(OCc6ccc(Cl)cc6F)n5)CC3)n4C[C@@H]3CCO3)cc12] |
| 120 | 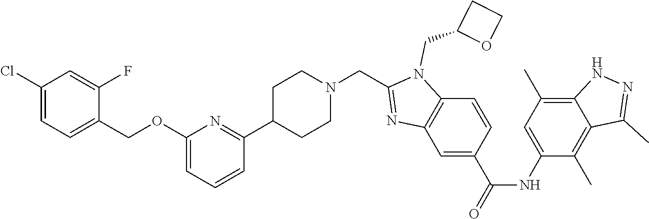 [SMILES: Cc1cc(NC(=O)c2ccc3c(c2)nc(CN2CCC(c4cccc(OCc5ccc(Cl)cc5F)n4)CC2)n3C[C@@H]2CCO2)c(C)c2c(C)n[nH]c12] |
| 121 | 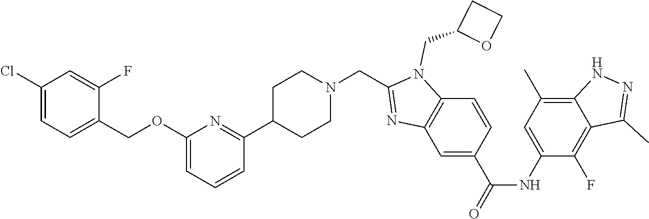 [SMILES: Cc1cc(NC(=O)c2ccc3c(c2)nc(CN2CCC(c4cccc(OCc5ccc(Cl)cc5F)n4)CC2)n3C[C@@H]2CCO2)c(F)c2c(C)n[nH]c12] |
| 122 | 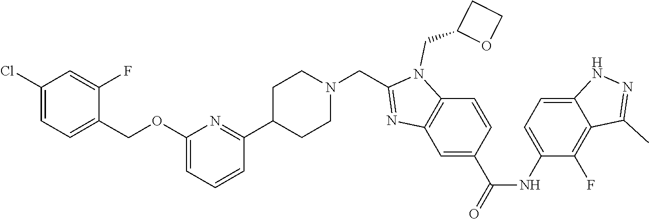 [SMILES: Cc1n[nH]c2ccc(NC(=O)c3ccc4c(c3)nc(CN3CCC(c5cccc(OCc6ccc(Cl)cc6F)n5)CC3)n4C[C@@H]3CCO3)c(F)c12] |
| 123 | 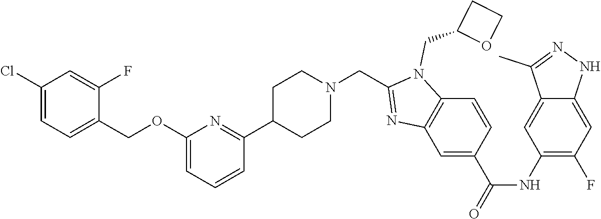 [SMILES: Cc1n[nH]c2cc(F)c(NC(=O)c3ccc4c(c3)nc(CN3CCC(c5cccc(OCc6ccc(Cl)cc6F)n5)CC3)n4C[C@@H]3CCO3)cc12] |
| 124 | 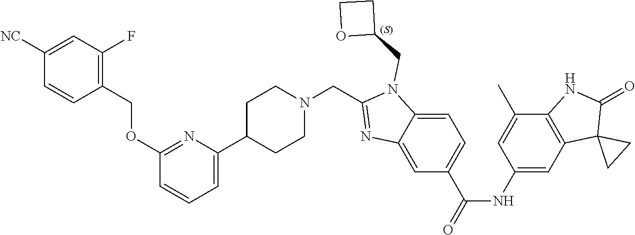 |
| 125 | 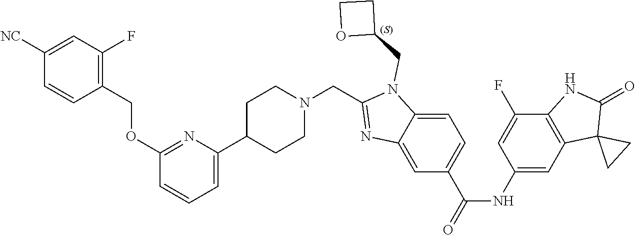 |
| 126 | 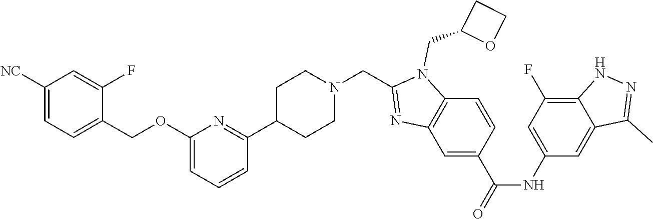 [SMILES: Cc1n[nH]c2c(F)cc(NC(=O)c3ccc4c(c3)nc(CN3CCC(c5cccc(OCc6ccc(C#N)cc6F)n5)CC3)n4C[C@@H]3CCO3)cc12] |
| 127 | 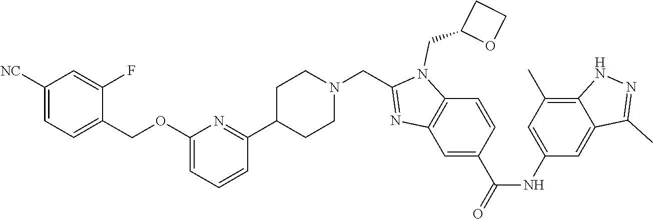 [SMILES: Cc1n[nH]c2c(C)cc(NC(=O)c3ccc4c(c3)nc(CN3CCC(c5cccc(OCc6ccc(C#N)cc6F)n5)CC3)n4C[C@@H]3CCO3)cc12] |
| 128 | 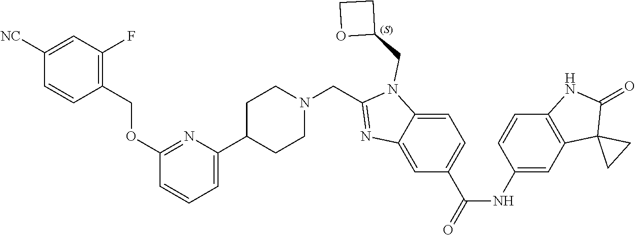 |
| 129 | 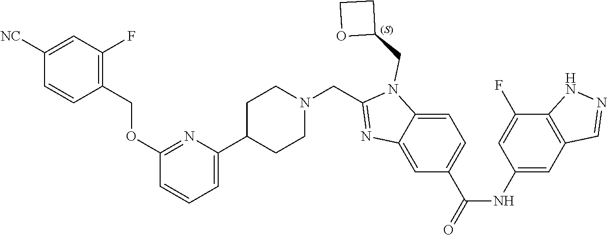 |
| 130 | 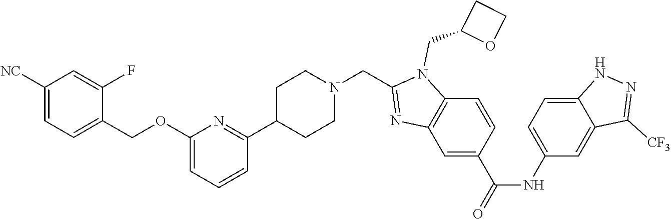 [SMILES: N#Cc1ccc(COc2cccc(C3CCN(Cc4nc5cc(C(=O)Nc6ccc7[nH]nc(C(F)(F)F)c7c6)ccc5n4C[C@@H]4CCO4)CC3)n2)c(F)c1] |
| 131 | 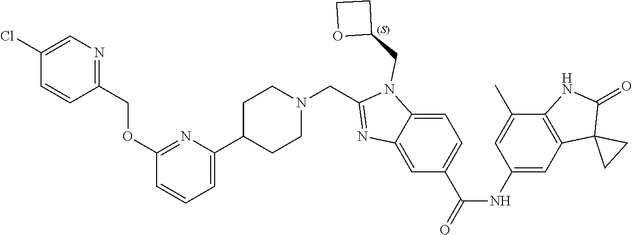 |
| 132 | 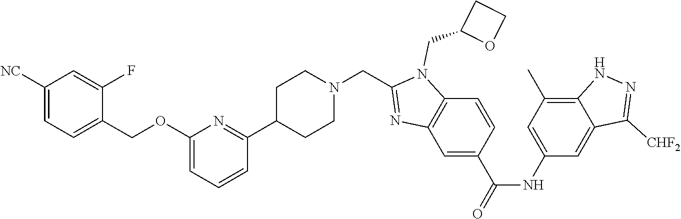 [SMILES: Cc1cc(NC(=O)c2ccc3c(c2)nc(CN2CCC(c4cccc(OCc5ccc(C#N)cc5F)n4)CC2)n3C[C@@H]2CCO2)cc2c(C(F)F)n[nH]c12] |
| 133 | 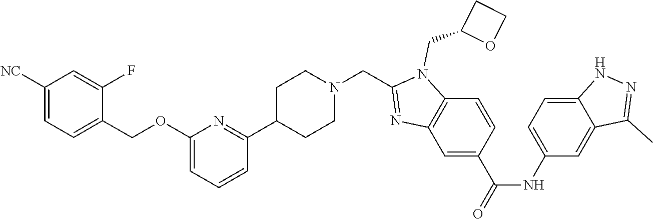 [SMILES: Cc1n[nH]c2ccc(NC(=O)c3ccc4c(c3)nc(CN3CCC(c5cccc(OCc6ccc(C#N)cc6F)n5)CC3)n4C[C@@H]3CCO3)cc12] |
| 134 | 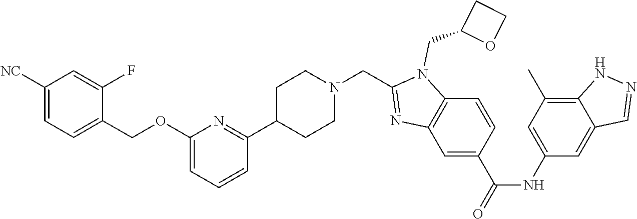 [SMILES: Cc1cc(NC(=O)c2ccc3c(c2)nc(CN2CCC(c4cccc(OCc5ccc(C#N)cc5F)n4)CC2)n3C[C@@H]2CCO2)cc2cn[nH]c12] |
| 135 | 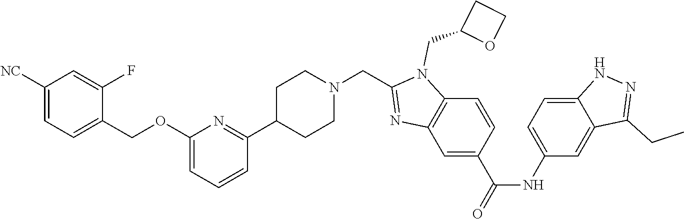 [SMILES: CCc1n[nH]c2ccc(NC(=O)c3ccc4c(c3)nc(CN3CCC(c5cccc(OCc6ccc(C#N)cc6F)n5)CC3)n4C[C@@H]3CCO3)cc12] |
| 136 | 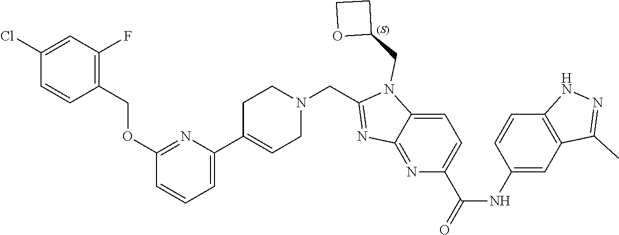 |
| 137 | 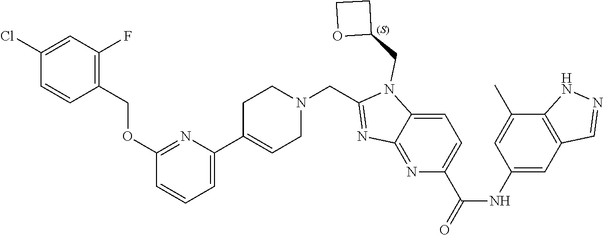 |
| 138 | 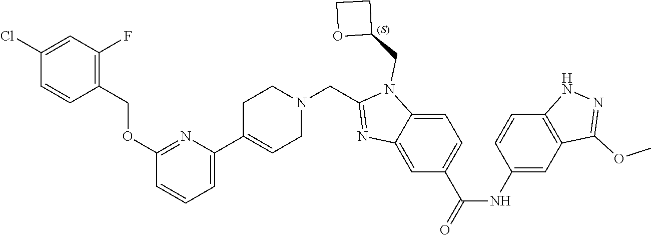 |
| 139 | 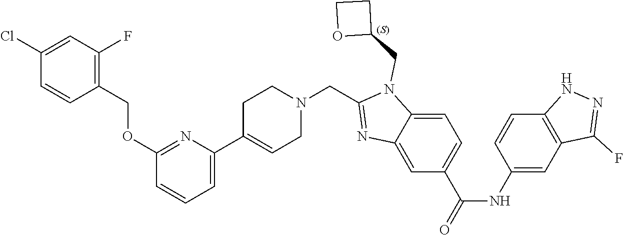 |
| 140 | 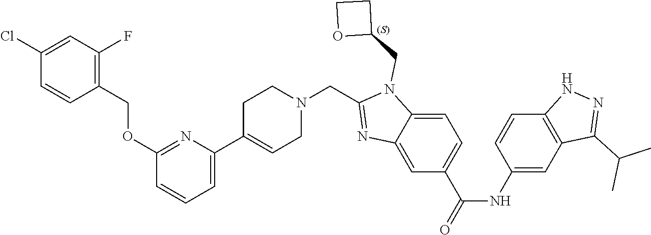 |
| 141 | 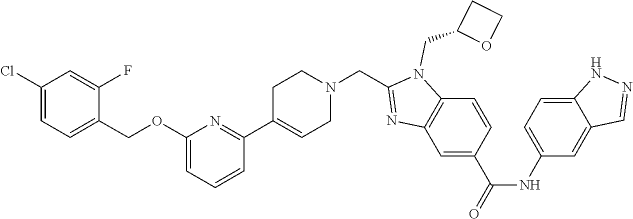 [SMILES: O=C(Nc1ccc2[nH]ncc2c1)c1ccc2c(c1)nc(CN1CC=C(c3cccc(OCc4ccc(Cl)cc4F)n3)CC1)n2C[C@@H]1CCO1] |
| 142 | 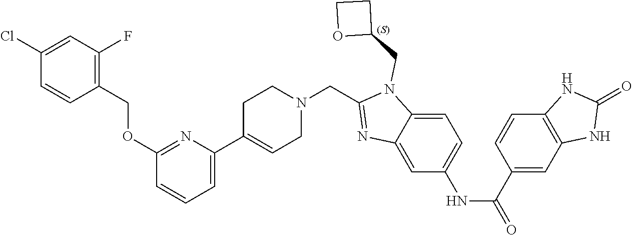 |
| 143 | 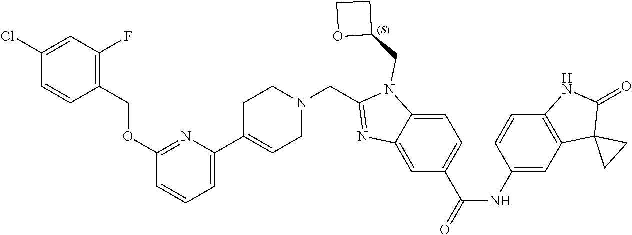 |
| 144 | 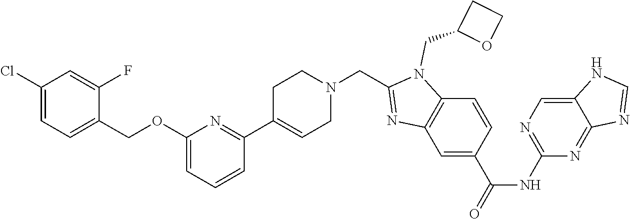 [SMILES: O=C(Nc1ncc2[nH]cnc2n1)c1ccc2c(c1)nc(CN1CC=C(c3cccc(OCc4ccc(Cl)cc4F)n3)CC1)n2C[C@@H]1CCO1] |
| 145 | 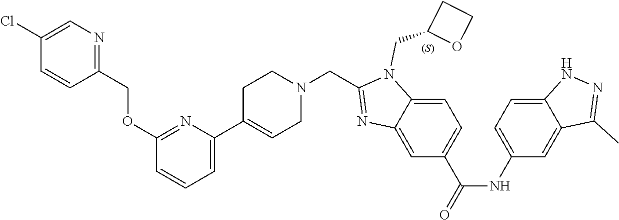 |
| 146 | 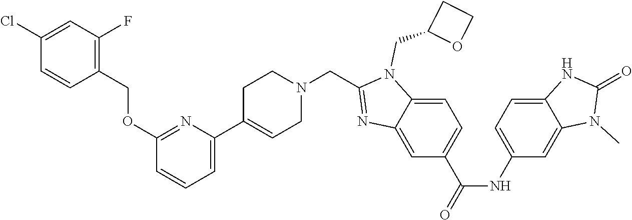 [SMILES: Cn1c(=O)[nH]c2ccc(NC(=O)c3ccc4c(c3)nc(CN3CC=C(c5cccc(OCc6ccc(Cl)cc6F)n5)CC3)n4C[C@@H]3CCO3)cc21] |
| 147 | 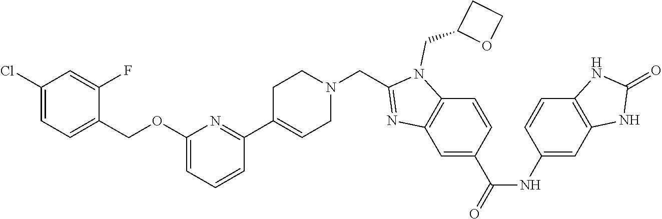 [SMILES: O=C(Nc1ccc2[nH]c(=O)[nH]c2c1)c1ccc2c(c1)nc(CN1CC=C(c3cccc(OCc4ccc(Cl)cc4F)n3)CC1)n2C[C@@H]1CCO1] |
| 148 | 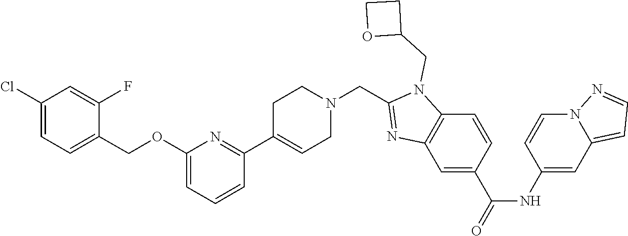 [SMILES: O=C(Nc1ccn2nccc2c1)c1ccc2c(c1)nc(CN1CC=C(c3cccc(OCc4ccc(Cl)cc4F)n3)CC1)n2CC1CCO1] |
| 149 | 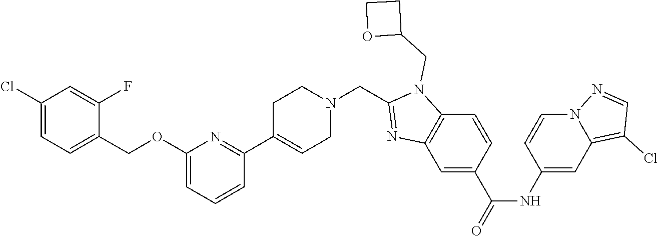 [SMILES: O=C(Nc1ccn2ncc(Cl)c2c1)c1ccc2c(c1)nc(CN1CC=C(c3cccc(OCc4ccc(Cl)cc4F)n3)CC1)n2CC1CCO1] |
| 150 | 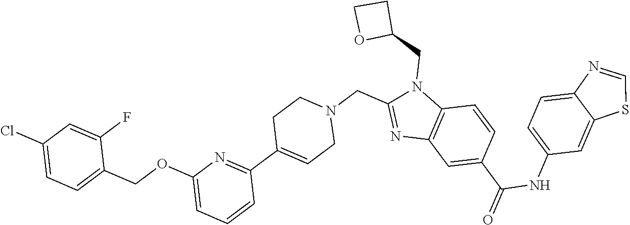 [SMILES: O=C(Nc1ccc2ncsc2c1)c1ccc2c(c1)nc(CN1CC=C(c3cccc(OCc4ccc(Cl)cc4F)n3)CC1)n2C[C@@H]1CCO1] |
| 151 | 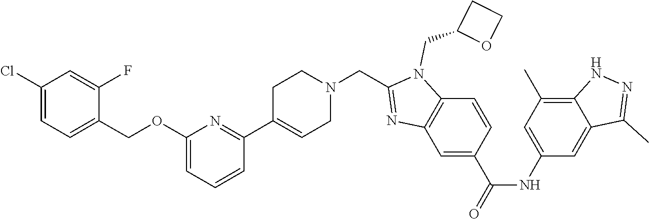 [SMILES: Cc1n[nH]c2c(C)cc(NC(=O)c3ccc4c(c3)nc(CN3CC=C(c5cccc(OCc6ccc(Cl)cc6F)n5)CC3)n4C[C@@H]3CCO3)cc12] |
| 152 | 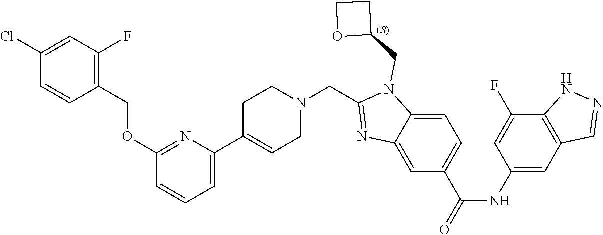 |
| 153 | 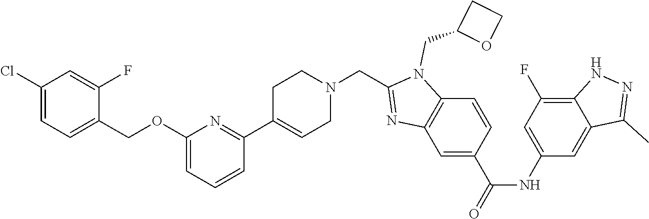 [SMILES: Cc1n[nH]c2c(F)cc(NC(=O)c3ccc4c(c3)nc(CN3CC=C(c5cccc(OCc6ccc(Cl)cc6F)n5)CC3)n4C[C@@H]3CCO3)cc12] |
| 154 | 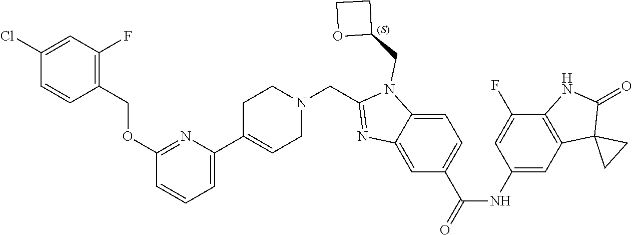 |
| 155 | 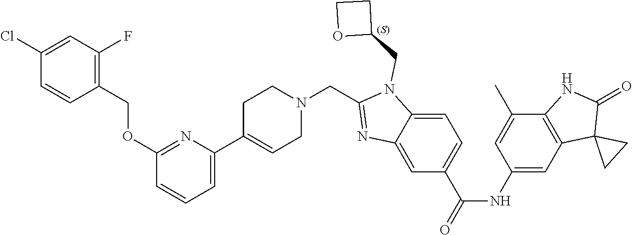 |
| 156 | 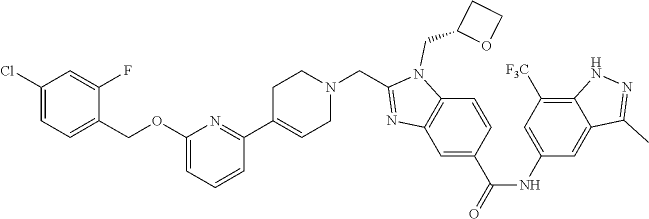 [SMILES: Cc1n[nH]c2c(C(F)(F)F)cc(NC(=O)c3ccc4c(c3)nc(CN3CC=C(c5cccc(OCc6ccc(Cl)cc6F)n5)CC3)n4C[C@@H]3CCO3)cc12] |
| 157 | 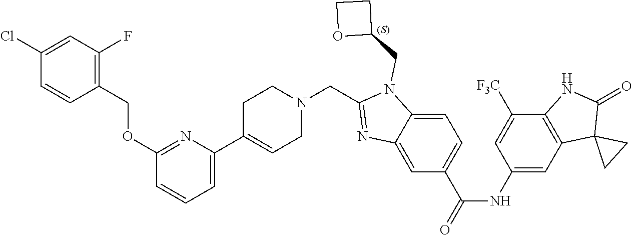 |
| 158 | 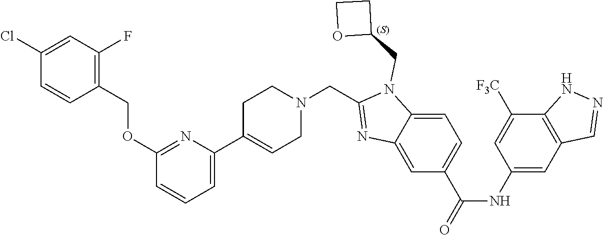 |
| 159 | 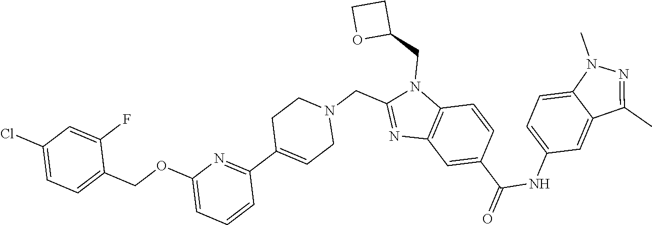 [SMILES: Cc1nn(C)c2ccc(NC(=O)c3ccc4c(c3)nc(CN3CC=C(c5cccc(OCc6ccc(Cl)cc6F)n5)CC3)n4C[C@@H]3CCO3)cc12] |
| 160 | 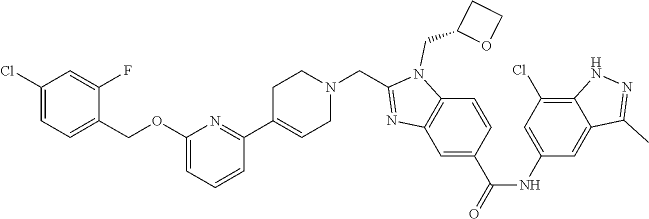 [SMILES: Cc1n[nH]c2c(Cl)cc(NC(=O)c3ccc4c(c3)nc(CN3CC=C(c5cccc(OCc6ccc(Cl)cc6F)n5)CC3)n4C[C@@H]3CCO3)cc12] |
| 161 | 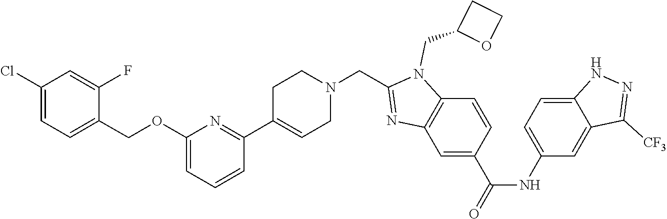 [SMILES: O=C(Nc1ccc2[nH]nc(C(F)(F)F)c2c1)c1ccc2c(c1)nc(CN1CC=C(c3cccc(OCc4ccc(Cl)cc4F)n3)CC1)n2C[C@@H]1CCO1] |
| 162 | 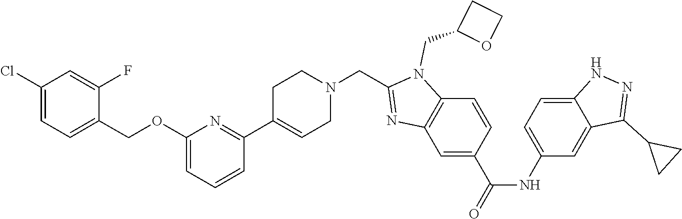 [SMILES: O=C(Nc1ccc2[nH]nc(C3CC3)c2c1)c1ccc2c(c1)nc(CN1CC=C(c3cccc(OCc4ccc(Cl)cc4F)n3)CC1)n2C[C@@H]1CCO1] |
| 163 | 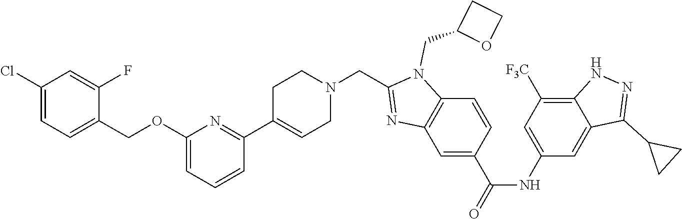 [SMILES: O=C(Nc1cc(C(F)(F)F)c2[nH]nc(C3CC3)c2c1)c1ccc2c(c1)nc(CN1CC=C(c3cccc(OCc4ccc(Cl)cc4F)n3)CC1)n2C[C@@H]1CCO1] |
| 164 | 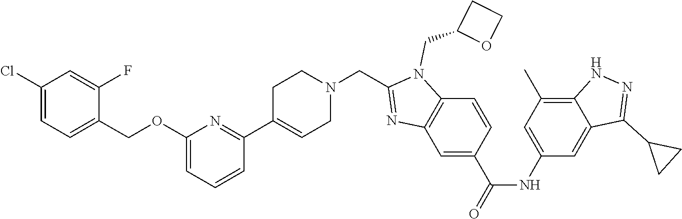 [SMILES: Cc1cc(NC(=O)c2ccc3c(c2)nc(CN2CC=C(c4cccc(OCc5ccc(Cl)cc5F)n4)CC2)n3C[C@@H]2CCO2)cc2c(C3CC3)n[nH]c12] |
| 165 | 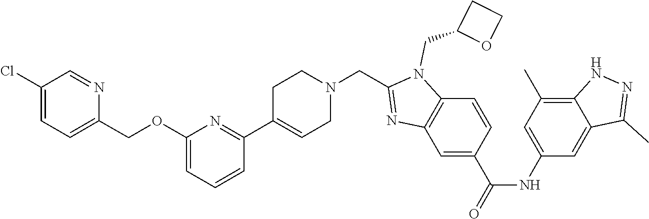 [SMILES: Cc1n[nH]c2c(C)cc(NC(=O)c3ccc4c(c3)nc(CN3CC=C(c5cccc(OCc6ccc(Cl)cn6)n5)CC3)n4C[C@@H]3CCO3)cc12] |
| 166 | 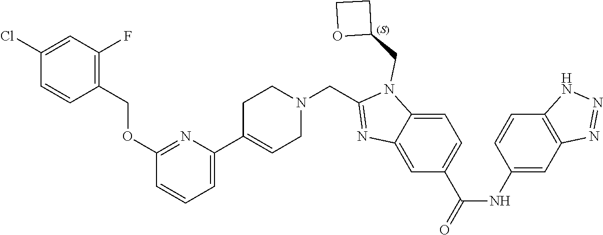 |
| 167 | 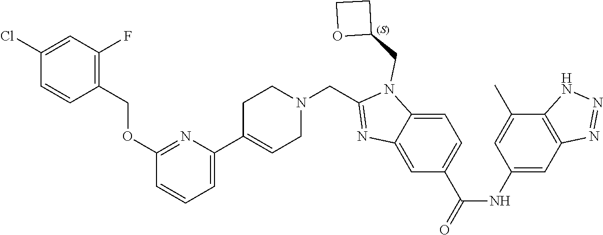 |
| 168 | 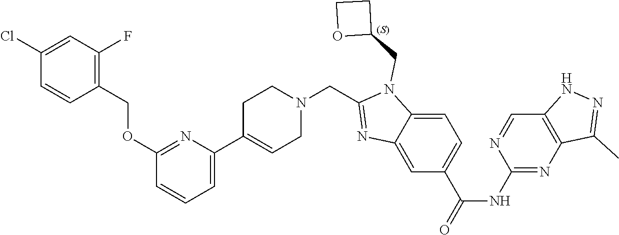 |
| 169 | 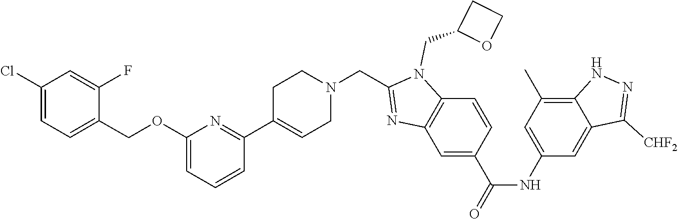 [SMILES: Cc1cc(NC(=O)c2ccc3c(c2)nc(CN2CC=C(c4cccc(OCc5ccc(Cl)cc5F)n4)CC2)n3C[C@@H]2CCO2)cc2c(C(F)F)n[nH]c12] |
| 170 | 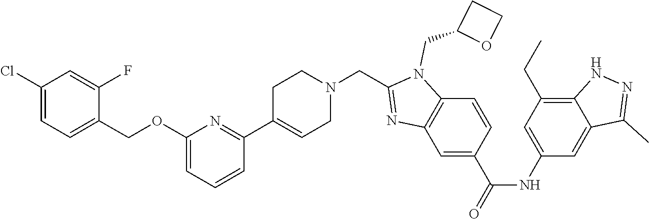 [SMILES: CCc1cc(NC(=O)c2ccc3c(c2)nc(CN2CC=C(c4cccc(OCc5ccc(Cl)cc5F)n4)CC2)n3C[C@@H]2CCO2)cc2c(C)n[nH]c12] |
| 171 | 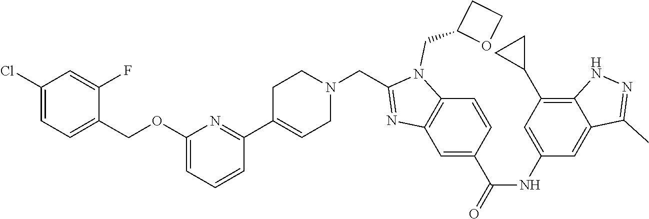 [SMILES: Cc1n[nH]c2c(C3CC3)cc(NC(=O)c3ccc4c(c3)nc(CN3CC=C(c5cccc(OCc6ccc(Cl)cc6F)n5)CC3)n4C[C@@H]3CCO3)cc12] |
| 172 | 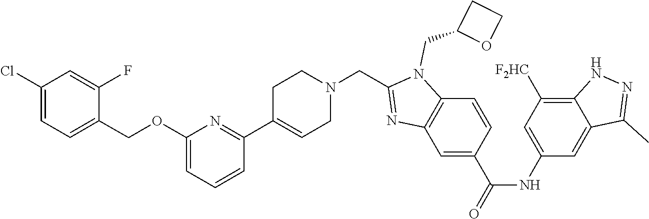 [SMILES: Cc1n[nH]c2c(C(F)F)cc(NC(=O)c3ccc4c(c3)nc(CN3CC=C(c5cccc(OCc6ccc(Cl)cc6F)n5)CC3)n4C[C@@H]3CCO3)cc12] |
| 173 | 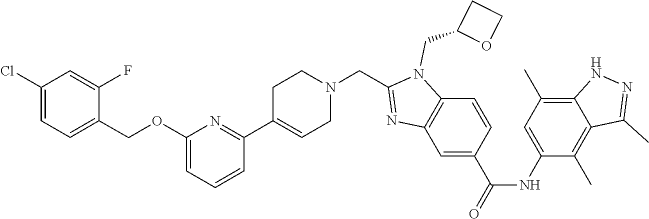 [SMILES: Cc1cc(NC(=O)c2ccc3c(c2)nc(CN2CC=C(c4cccc(OCc5ccc(Cl)cc5F)n4)CC2)n3C[C@@H]2CCO2)c(C)c2c(C)n[nH]c12] |
| 174 | 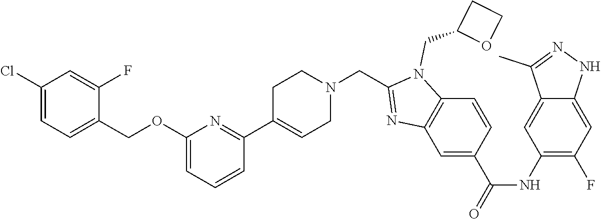 [SMILES: Cc1n[nH]c2cc(F)c(NC(=O)c3ccc4c(c3)nc(CN3CC=C(c5cccc(OCc6ccc(Cl)cc6F)n5)CC3)n4C[C@@H]3CCO3)cc12] |
| 175 | 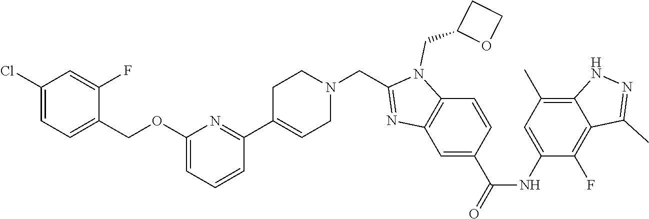 [SMILES: Cc1cc(NC(=O)c2ccc3c(c2)nc(CN2CC=C(c4cccc(OCc5ccc(Cl)cc5F)n4)CC2)n3C[C@@H]2CCO2)c(F)c2c(C)n[nH]c12] |
| 176 | 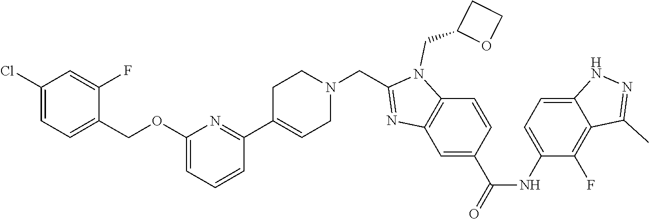 [SMILES: Cc1n[nH]c2ccc(NC(=O)c3ccc4c(c3)nc(CN3CC=C(c5cccc(OCc6ccc(Cl)cc6F)n5)CC3)n4C[C@@H]3CCO3)c(F)c12] |
| 177 | 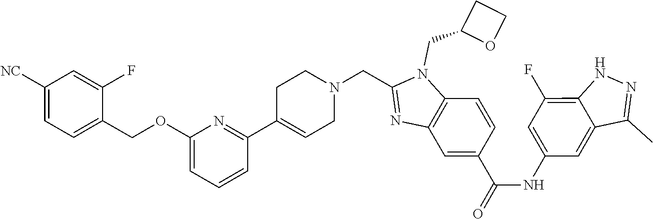 [SMILES: Cc1n[nH]c2c(F)cc(NC(=O)c3ccc4c(c3)nc(CN3CC=C(c5cccc(OCc6ccc(C#N)cc6F)n5)CC3)n4C[C@@H]3CCO3)cc12] |
| 178 | 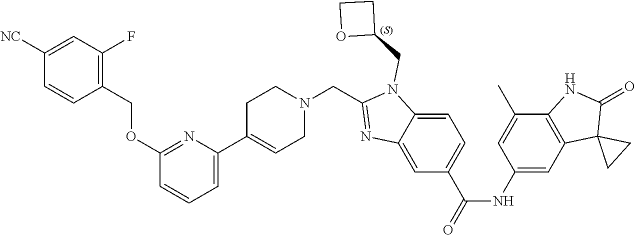 |
| 179 | 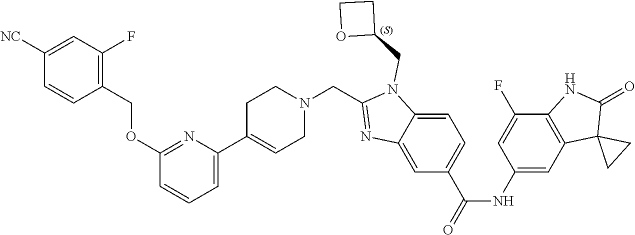 |
| 180 | 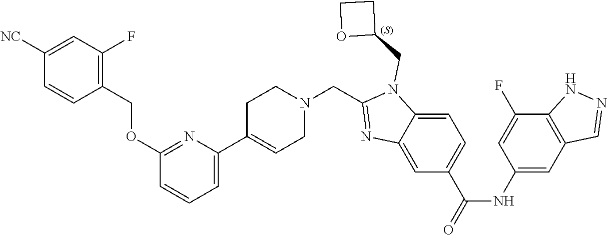 |
| 181 | 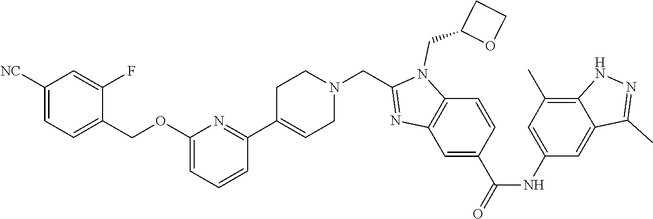 [SMILES: Cc1n[nH]c2c(C)cc(NC(=O)c3ccc4c(c3)nc(CN3CC=C(c5cccc(OCc6ccc(C#N)cc6F)n5)CC3)n4C[C@@H]3CCO3)cc12] |
| 182 | 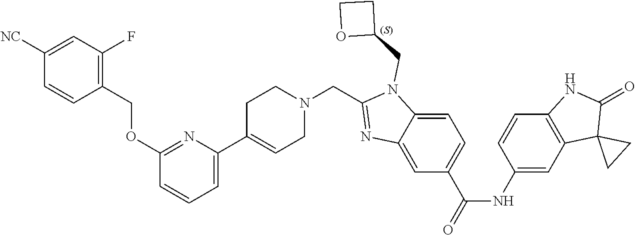 |
| 183 | 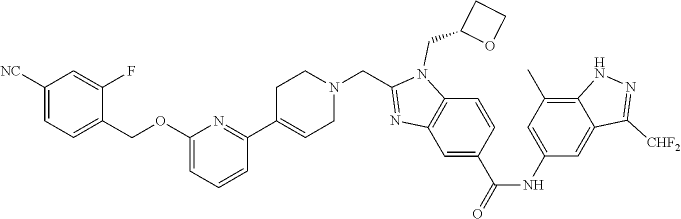 [SMILES: Cc1cc(NC(=O)c2ccc3c(c2)nc(CN2CC=C(c4cccc(OCc5ccc(C#N)cc5F)n4)CC2)n3C[C@@H]2CCO2)cc2c(C(F)F)n[nH]c12] |
| 184 | 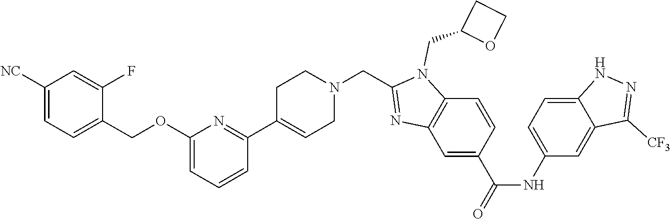 [SMILES: N#Cc1ccc(COc2cccc(C3=CCN(Cc4nc5cc(C(=O)Nc6ccc7[nH]nc(C(F)(F)F)c7c6)ccc5n4C[C@@H]4CCO4)CC3)n2)c(F)c1] |
| 185 | 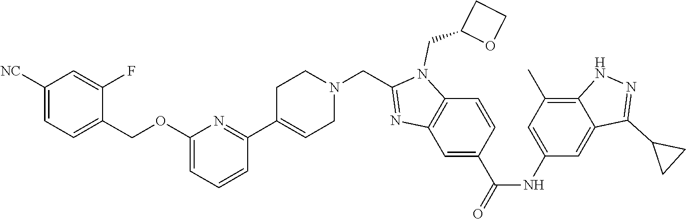 [SMILES: Cc1cc(NC(=O)c2ccc3c(c2)nc(CN2CC=C(c4cccc(OCc5ccc(C#N)cc5F)n4)CC2)n3C[C@@H]2CCO2)cc2c(C3CC3)n[nH]c12] |
| 186 | 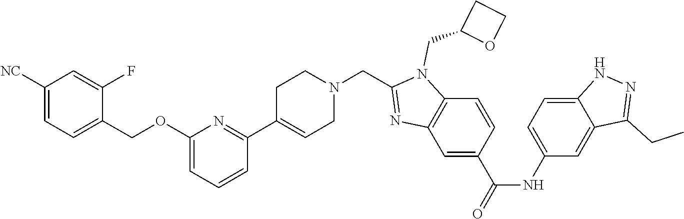 [SMILES: CCc1n[nH]c2ccc(NC(=O)c3ccc4c(c3)nc(CN3CC=C(c5cccc(OCc6ccc(C#N)cc6F)n5)CC3)n4C[C@@H]3CCO3)cc12] |
| 187 | 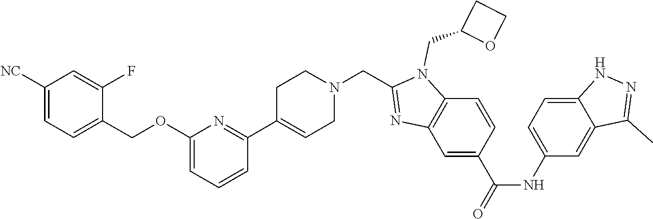 [SMILES: Cc1n[nH]c2ccc(NC(=O)c3ccc4c(c3)nc(CN3CC=C(c5cccc(OCc6ccc(C#N)cc6F)n5)CC3)n4C[C@@H]3CCO3)cc12] |
| 188 | 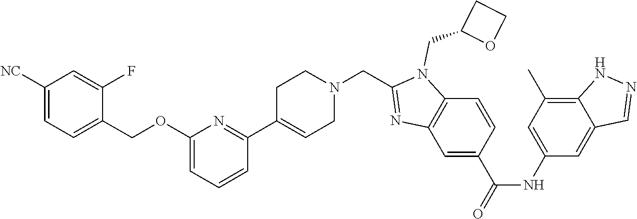 [SMILES: Cc1cc(NC(=O)c2ccc3c(c2)nc(CN2CC=C(c4cccc(OCc5ccc(C#N)cc5F)n4)CC2)n3C[C@@H]2CCO2)cc2cn[nH]c12] |
| 189 | 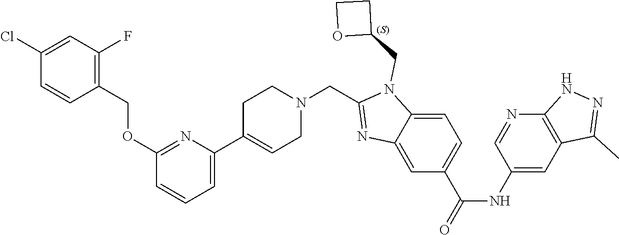 |
| 190 | 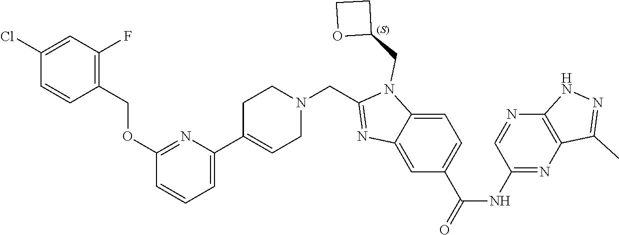 |
| 191 | 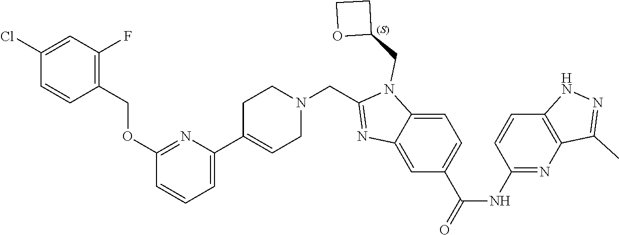 |
| 192 | 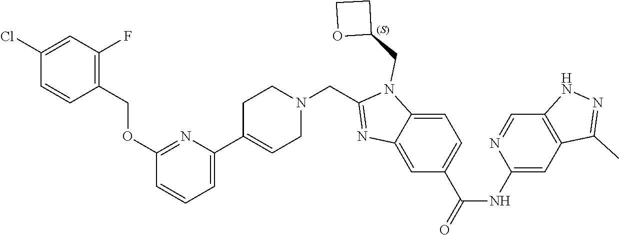 |
| 193 | 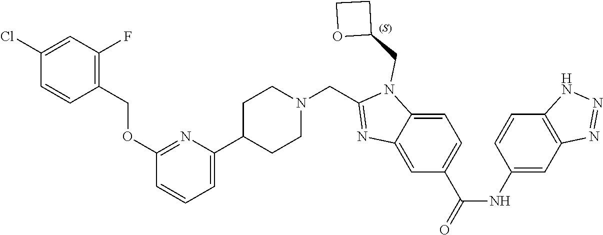 |
| 194 | 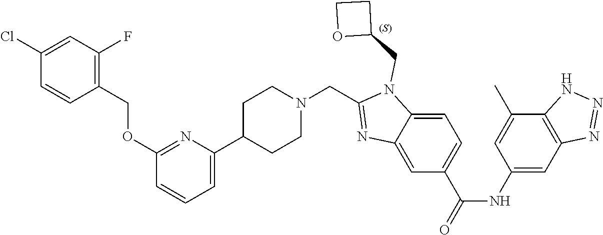 |
| 195 | 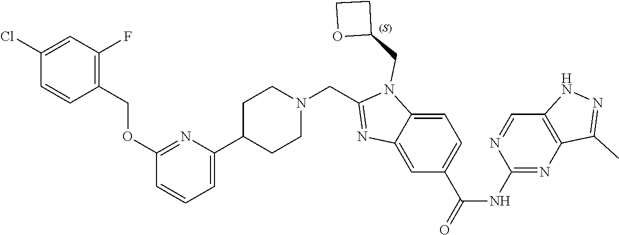 |
| 196 | 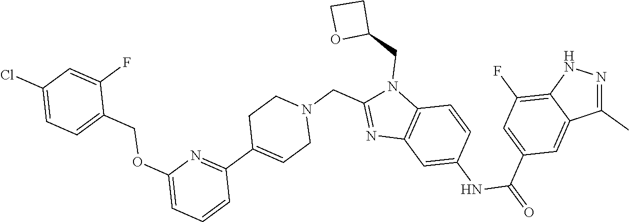 [SMILES: Cc1n[nH]c2c(F)cc(C(=O)Nc3ccc4c(c3)nc(CN3CC=C(c5cccc(OCc6ccc(Cl)cc6F)n5)CC3)n4C[C@@H]3CCO3)cc12] |
| 197 | 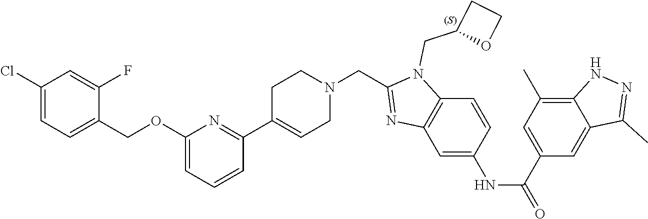 |
| 198 | 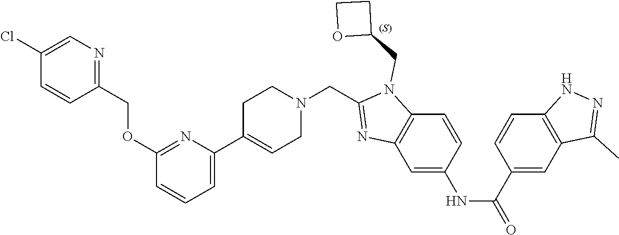 |
| 199 | 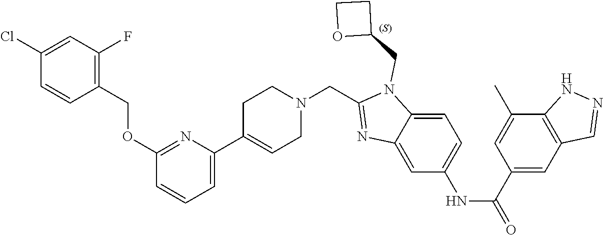 |
| 200 | 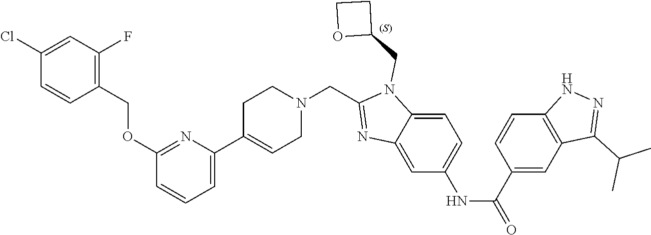 |
| 201 | 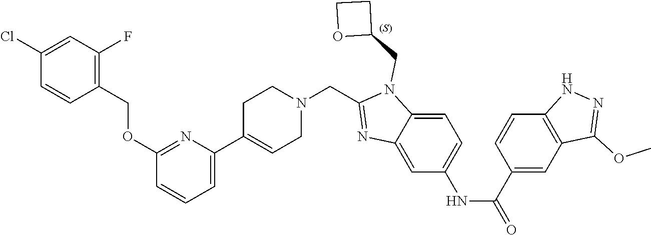 |
| 202 | 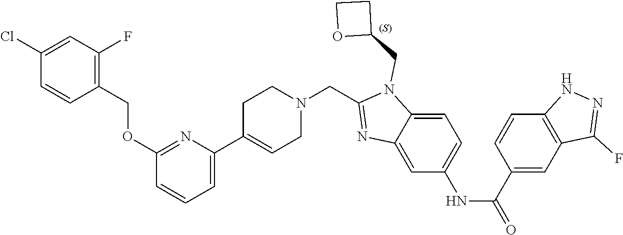 |
| 203 | 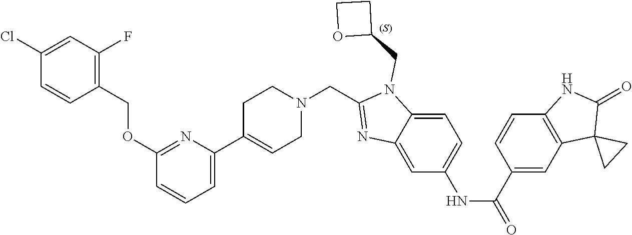 |
| 204 | 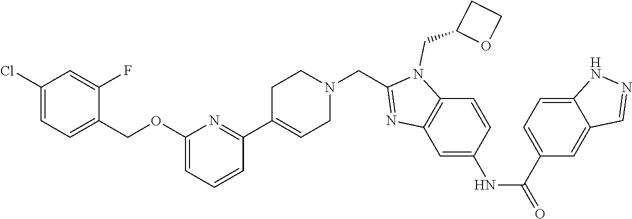 [SMILES: O=C(Nc1ccc2c(c1)nc(CN1CC=C(c3cccc(OCc4ccc(Cl)cc4F)n3)CC1)n2C[C@@H]1CCO1)c1ccc2[nH]ncc2c1] |
| 205 | 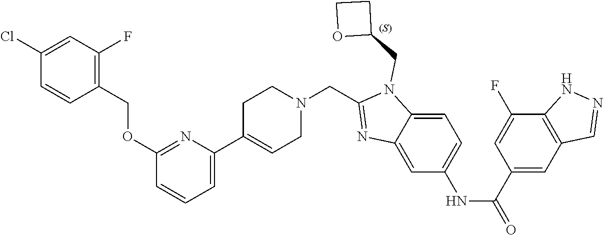 |
| 206 | 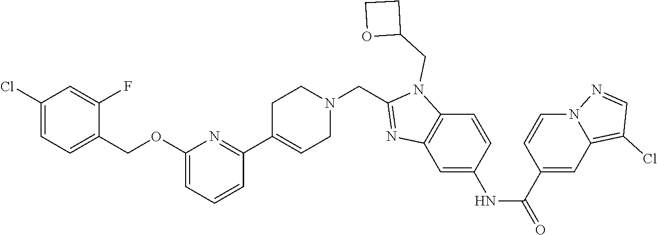 [SMILES: O=C(Nc1ccc2c(c1)nc(CN1CC=C(c3cccc(OCc4ccc(Cl)cc4F)n3)CC1)n2CC1CCO1)c1ccn2ncc(Cl)c2c1] |
| 207 | 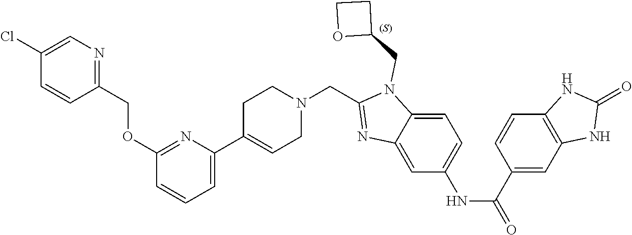 |
| 208 | 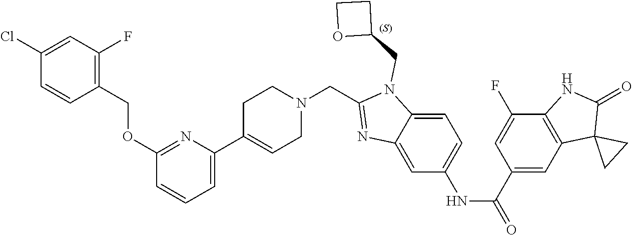 |
| 209 | 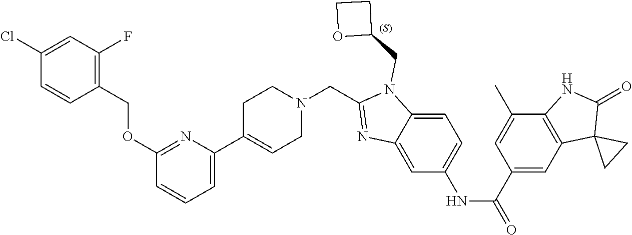 |
| 210 | 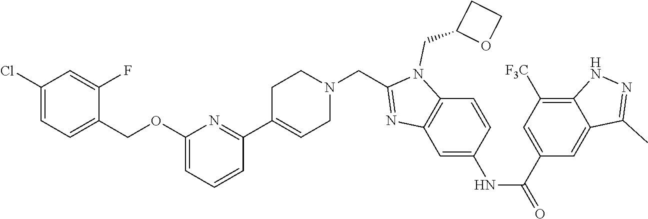 [SMILES: Cc1n[nH]c2c(C(F)(F)F)cc(C(=O)Nc3ccc4c(c3)nc(CN3CC=C(c5cccc(OCc6ccc(Cl)cc6F)n5)CC3)n4C[C@@H]3CCO3)cc12] |
| 211 | 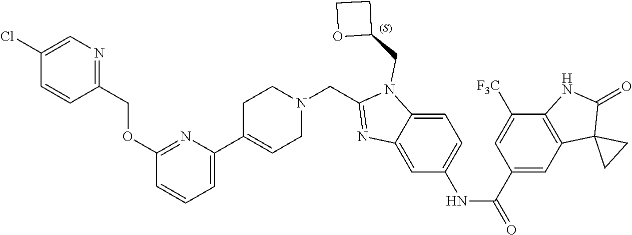 |
| 212 | 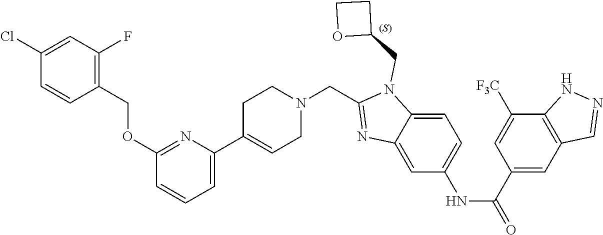 |
| 213 | 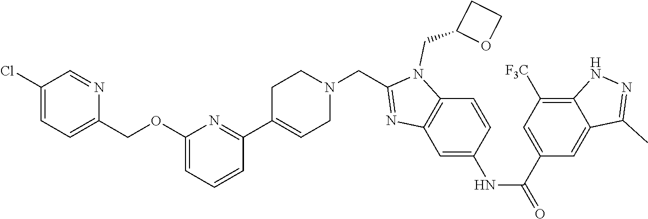 [SMILES: Cc1n[nH]c2c(C(F)(F)F)cc(C(=O)Nc3ccc4c(c3)nc(CN3CC=C(c5cccc(OCc6ccc(Cl)cn6)n5)CC3)n4C[C@@H]3CCO3)cc12] |
| 214 | 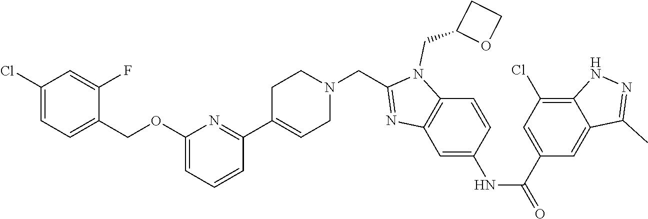 [SMILES: Cc1n[nH]c2c(Cl)cc(C(=O)Nc3ccc4c(c3)nc(CN3CC=C(c5cccc(OCc6ccc(Cl)cc6F)n5)CC3)n4C[C@@H]3CCO3)cc12] |
| 215 | 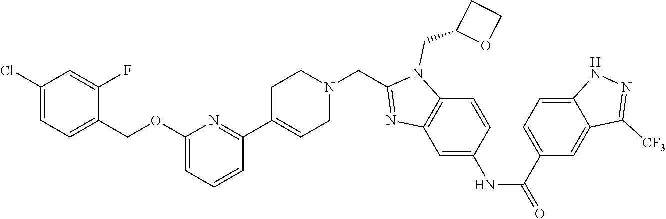 [SMILES: O=C(Nc1ccc2c(c1)nc(CN1CC=C(c3cccc(OCc4ccc(Cl)cc4F)n3)CC1)n2C[C@@H]1CCO1)c1ccc2[nH]nc(C(F)(F)F)c2c1] |
| 216 | 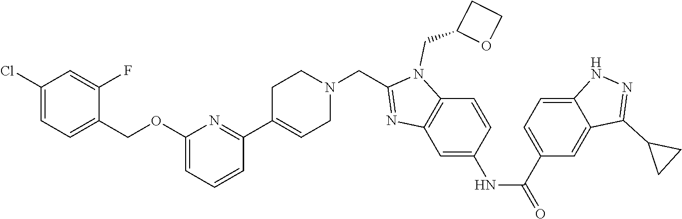 [SMILES: O=C(Nc1ccc2c(c1)nc(CN1CC=C(c3cccc(OCc4ccc(Cl)cc4F)n3)CC1)n2C[C@@H]1CCO1)c1ccc2[nH]nc(C3CC3)c2c1] |
| 217 | 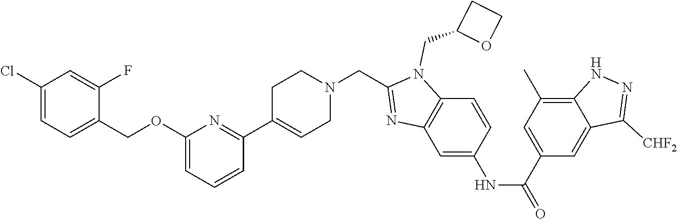 [SMILES: Cc1cc(C(=O)Nc2ccc3c(c2)nc(CN2CC=C(c4cccc(OCc5ccc(Cl)cc5F)n4)CC2)n3C[C@@H]2CCO2)cc2c(C(F)F)n[nH]c12] |
| 218 | 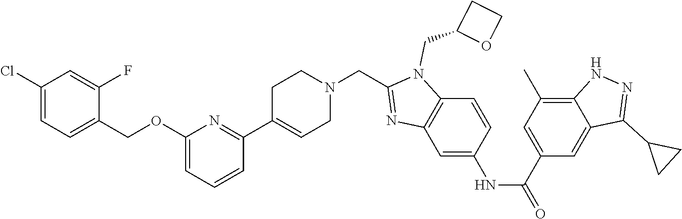 [SMILES: Cc1cc(C(=O)Nc2ccc3c(c2)nc(CN2CC=C(c4cccc(OCc5ccc(Cl)cc5F)n4)CC2)n3C[C@@H]2CCO2)cc2c(C3CC3)n[nH]c12] |
| 219 | 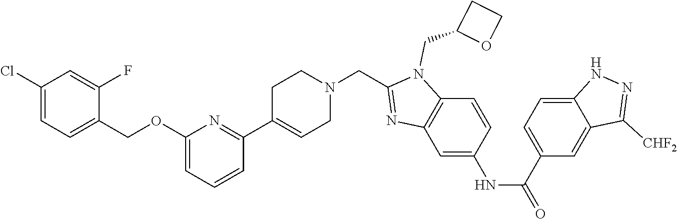 [SMILES: O=C(Nc1ccc2c(c1)nc(CN1CC=C(c3cccc(OCc4ccc(Cl)cc4F)n3)CC1)n2C[C@@H]1CCO1)c1ccc2[nH]nc(C(F)F)c2c1] |
| 220 | 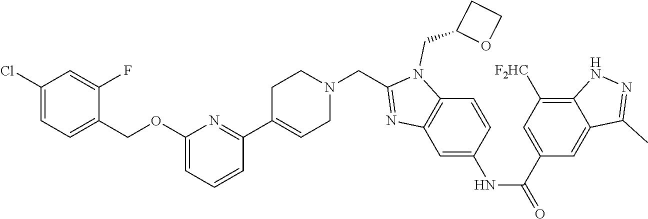 [SMILES: Cc1n[nH]c2c(C(F)F)cc(C(=O)Nc3ccc4c(c3)nc(CN3CC=C(c5cccc(OCc6ccc(Cl)cc6F)n5)CC3)n4C[C@@H]3CCO3)cc12] |
| 221 | 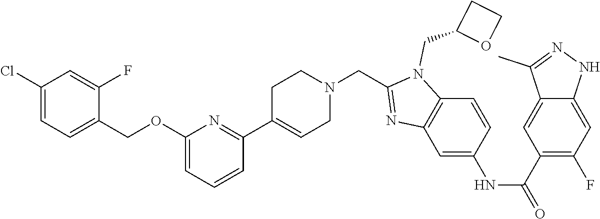 [SMILES: Cc1n[nH]c2cc(F)c(C(=O)Nc3ccc4c(c3)nc(CN3CC=C(c5cccc(OCc6ccc(Cl)cc6F)n5)CC3)n4C[C@@H]3CCO3)cc12] |
| 222 | 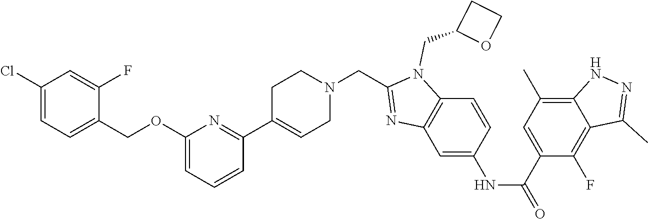 [SMILES: Cc1cc(C(=O)Nc2ccc3c(c2)nc(CN2CC=C(c4cccc(OCc5ccc(Cl)cc5F)n4)CC2)n3C[C@@H]2CCO2)c(F)c2c(C)n[nH]c12] |
| 223 | 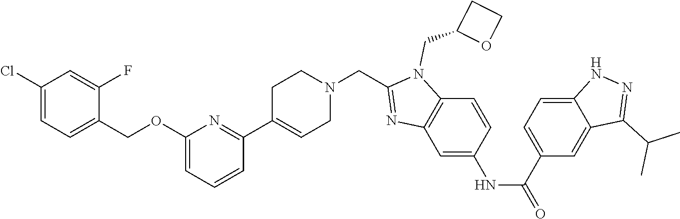 [SMILES: CC(C)c1n[nH]c2ccc(C(=O)Nc3ccc4c(c3)nc(CN3CC=C(c5cccc(OCc6ccc(Cl)cc6F)n5)CC3)n4C[C@@H]3CCO3)cc12] |
| 224 | 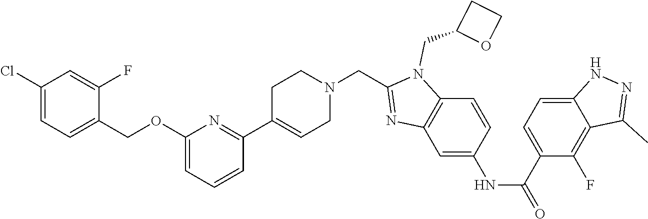 [SMILES: Cc1n[nH]c2ccc(C(=O)Nc3ccc4c(c3)nc(CN3CC=C(c5cccc(OCc6ccc(Cl)cc6F)n5)CC3)n4C[C@@H]3CCO3)c(F)c12] |
| 225 | 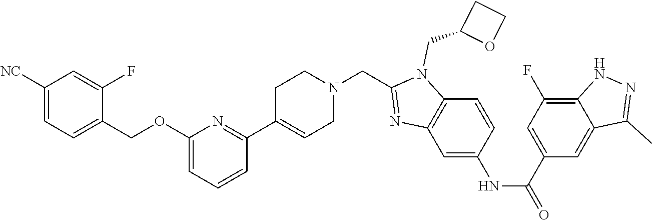 [SMILES: Cc1n[nH]c2c(F)cc(C(=O)Nc3ccc4c(c3)nc(CN3CC=C(c5cccc(OCc6ccc(C#N)cc6F)n5)CC3)n4C[C@@H]3CCO3)cc12] |
| 226 | 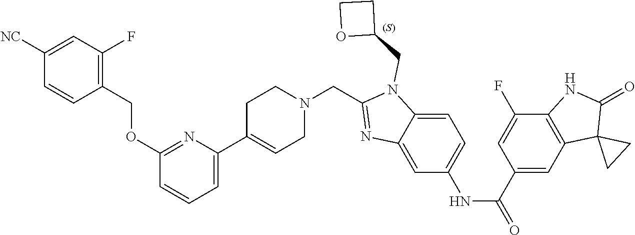 |
| 227 | 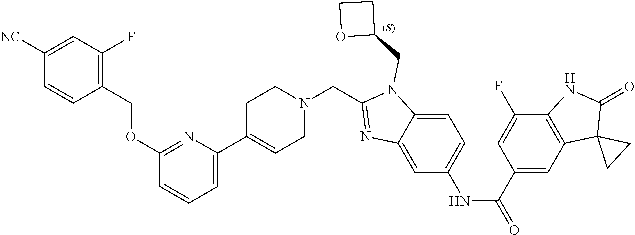 |
| 228 | 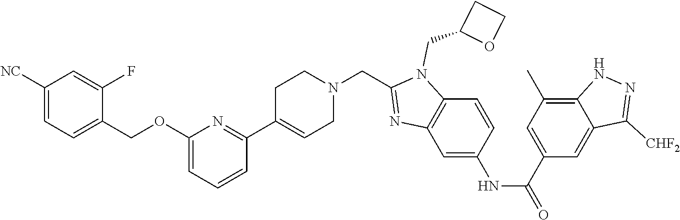 [SMILES: Cc1cc(C(=O)Nc2ccc3c(c2)nc(CN2CC=C(c4cccc(OCc5ccc(C#N)cc5F)n4)CC2)n3C[C@@H]2CCO2)cc2c(C(F)F)n[nH]c12] |
| 229 | 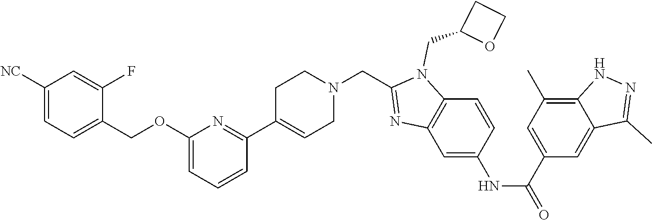 [SMILES: Cc1n[nH]c2c(C)cc(C(=O)Nc3ccc4c(c3)nc(CN3CC=C(c5cccc(OCc6ccc(C#N)cc6F)n5)CC3)n4C[C@@H]3CCO3)cc12] |
| 230 | 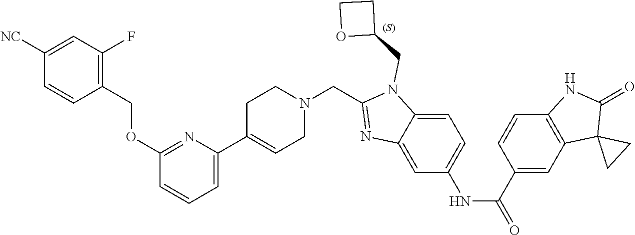 |
| 231 | 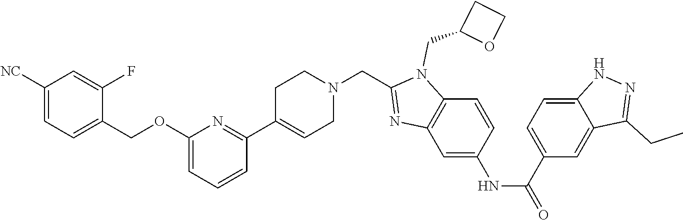 [SMILES: CCc1n[nH]c2ccc(C(=O)Nc3ccc4c(c3)nc(CN3CC=C(c5cccc(OCc6ccc(C#N)cc6F)n5)CC3)n4C[C@@H]3CCO3)cc12] |
| 232 | 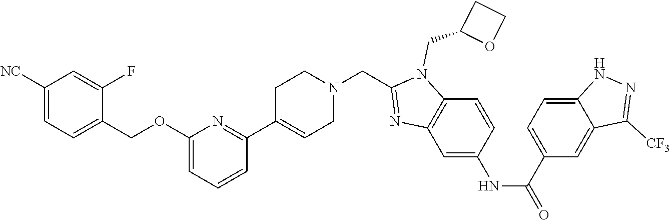 [SMILES: N#Cc1ccc(COc2cccc(C3=CCN(Cc4nc5cc(NC(=O)c6ccc7[nH]nc(C(F)(F)F)c7c6)ccc5n4C[C@@H]4CCO4)CC3)n2)c(F)c1] |
| 233 | 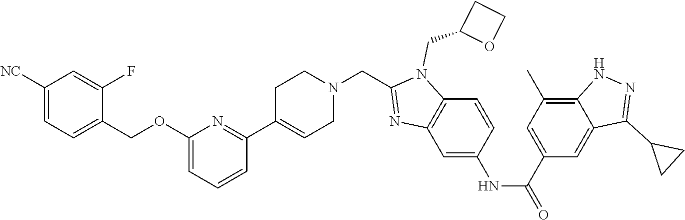 [SMILES: Cc1cc(C(=O)Nc2ccc3c(c2)nc(CN2CC=C(c4cccc(OCc5ccc(C#N)cc5F)n4)CC2)n3C[C@@H]2CCO2)cc2c(C3CC3)n[nH]c12] |
| 234 | 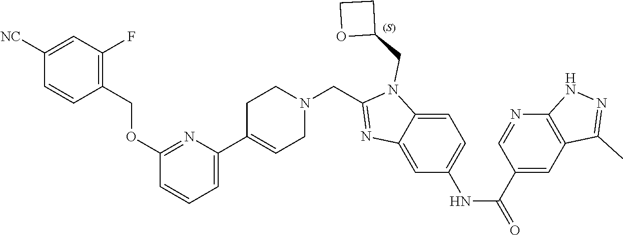 |
| 235 | 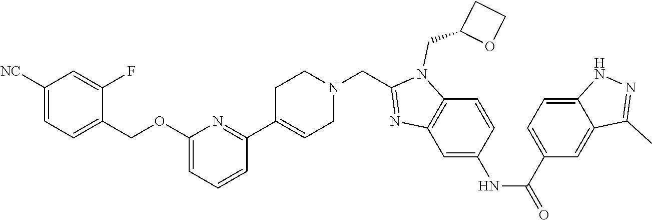 [SMILES: Cc1n[nH]c2ccc(C(=O)Nc3ccc4c(c3)nc(CN3CC=C(c5cccc(OCc6ccc(C#N)cc6F)n5)CC3)n4C[C@@H]3CCO3)cc12] |
| 236 | 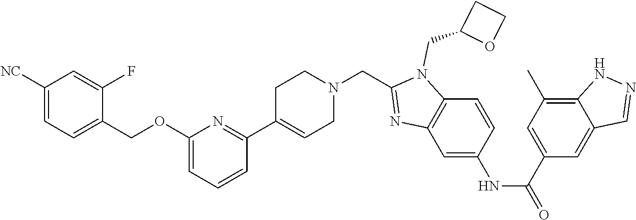 [SMILES: Cc1cc(C(=O)Nc2ccc3c(c2)nc(CN2CC=C(c4cccc(OCc5ccc(C#N)cc5F)n4)CC2)n3C[C@@H]2CCO2)cc2cn[nH]c12] |
| 237 | 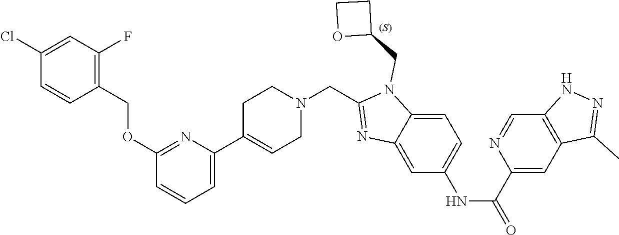 |
| 238 | 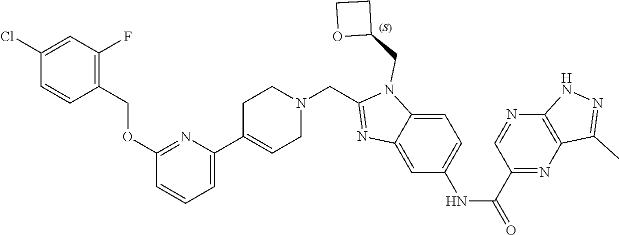 |
| 239 | 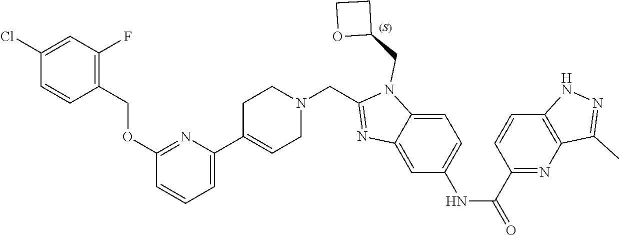 |
| 240 | 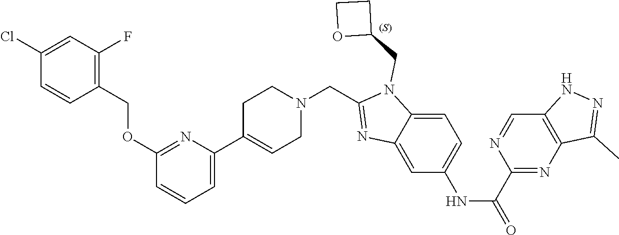 |
| 241 | 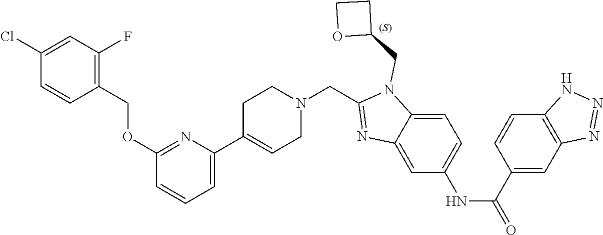 |
| 242 | 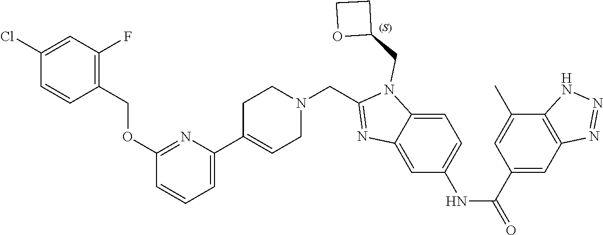 |
| 243 | 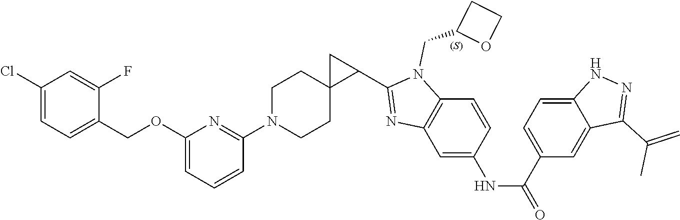 |
| 244 | 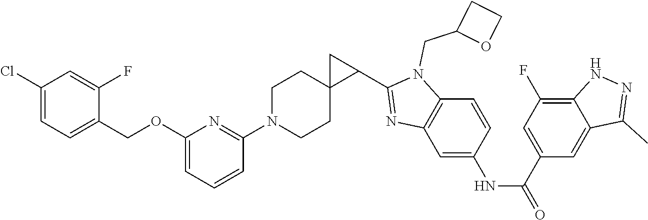 [SMILES: Cc1n[nH]c2c(F)cc(C(=O)Nc3ccc4c(c3)nc(C3CC35CCN(c3cccc(OCc6ccc(Cl)cc6F)n3)CC5)n4CC3CCO3)cc12] |
| 245 | 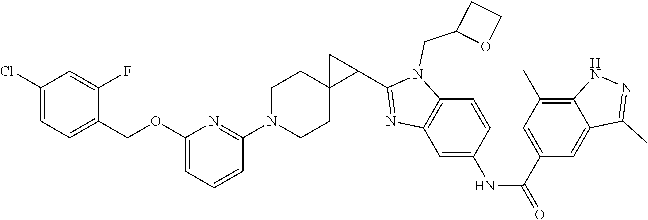 [SMILES: Cc1n[nH]c2c(C)cc(C(=O)Nc3ccc4c(c3)nc(C3CC35CCN(c3cccc(OCc6ccc(Cl)cc6F)n3)CC5)n4CC3CCO3)cc12] |
| 246 | 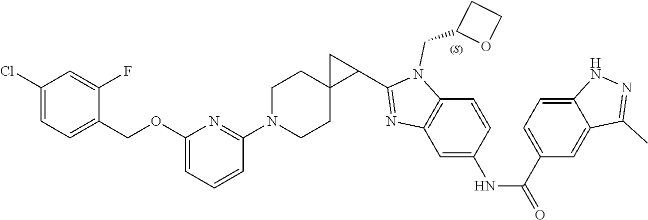 |
| 247 | 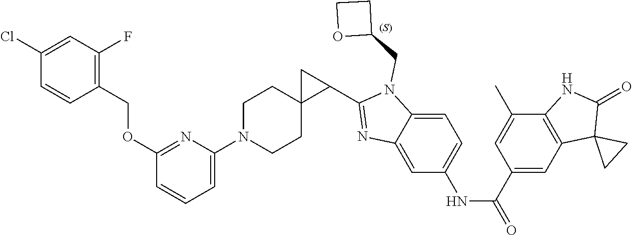 |
| 248 | 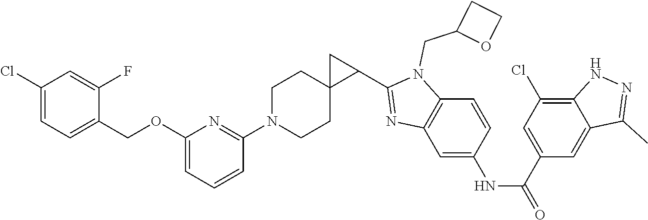 [SMILES: Cc1n[nH]c2c(Cl)cc(C(=O)Nc3ccc4c(c3)nc(C3CC35CCN(c3cccc(OCc6ccc(Cl)cc6F)n3)CC5)n4CC3CCO3)cc12] |
| 249 | 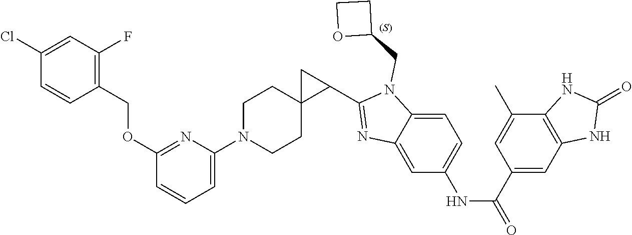 |
| 250 | 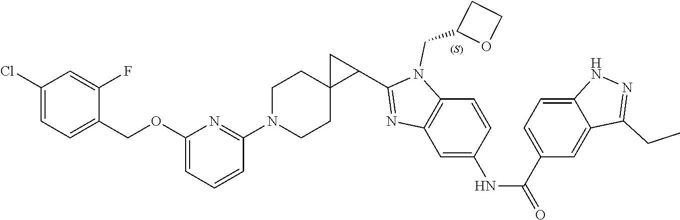 |
| 251 | 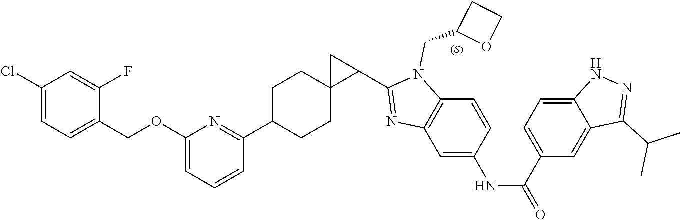 |
| 252 | 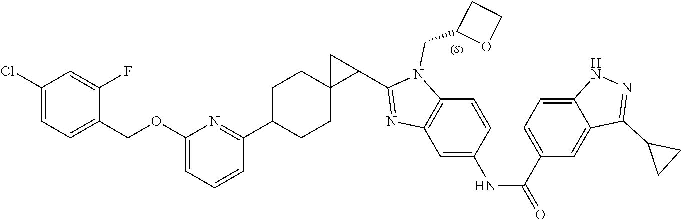 |
| 253 | 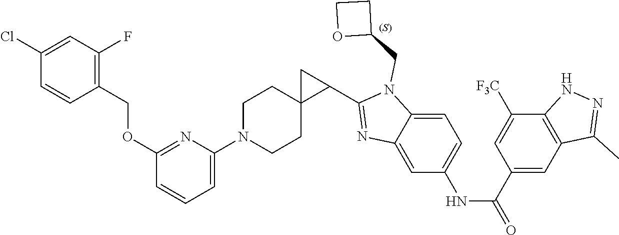 |
| 254 | 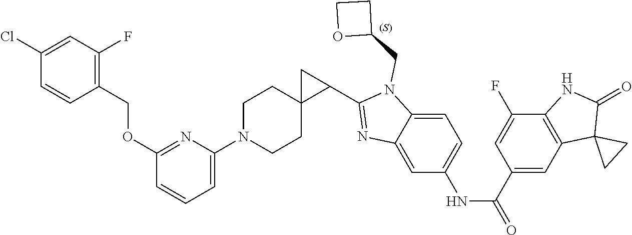 |
| 255 | 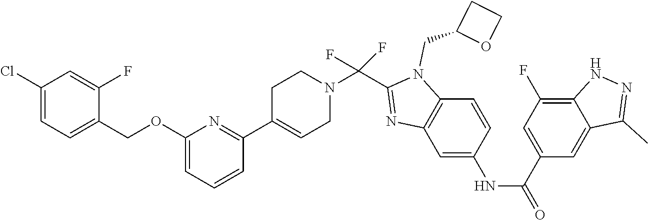 [SMILES: Cc1n[nH]c2c(F)cc(C(=O)Nc3ccc4c(c3)nc(C(F)(F)N3CC=C(c5cccc(OCc6ccc(Cl)cc6F)n5)CC3)n4C[C@@H]3CCO3)cc12] |
| 256 | 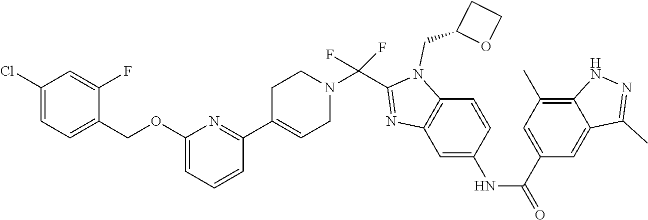 [SMILES: Cc1n[nH]c2c(C)cc(C(=O)Nc3ccc4c(c3)nc(C(F)(F)N3CC=C(c5cccc(OCc6ccc(Cl)cc6F)n5)CC3)n4C[C@@H]3CCO3)cc12] |
| 257 | 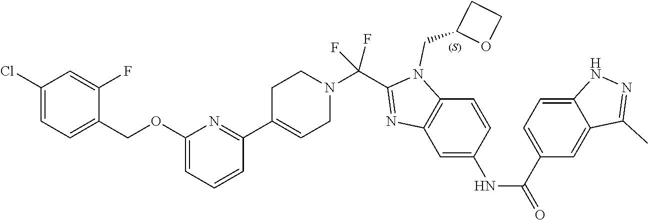 |
| 258 | 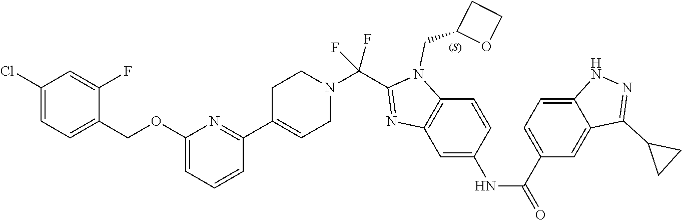 |
| 259 | 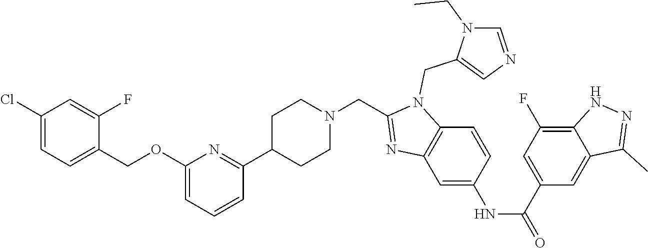 [SMILES: CCn1cncc1Cn1c(CN2CCC(c3cccc(OCc4ccc(Cl)cc4F)n3)CC2)nc2cc(NC(=O)c3cc(F)c4[nH]nc(C)c4c3)ccc21] |
| 260 | 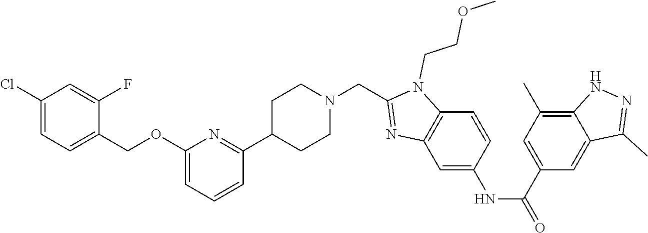 [SMILES: COCCn1c(CN2CCC(c3cccc(OCc4ccc(Cl)cc4F)n3)CC2)nc2cc(NC(=O)c3cc(C)c4[nH]nc(C)c4c3)ccc21] |
| 261 | 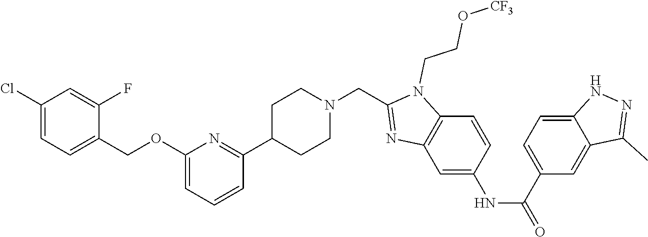 [SMILES: Cc1n[nH]c2ccc(C(=O)Nc3ccc4c(c3)nc(CN3CCC(c5cccc(OCc6ccc(Cl)cc6F)n5)CC3)n4CCOC(F)(F)F)cc12] |
| 262 | 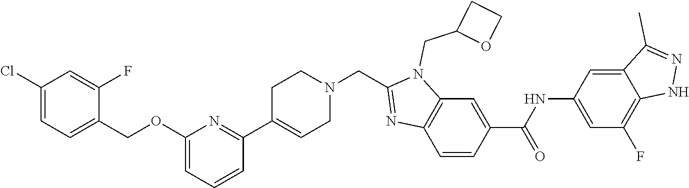 [SMILES: Cc1n[nH]c2c(F)cc(NC(=O)c3ccc4nc(CN5CC=C(c6cccc(OCc7ccc(Cl)cc7F)n6)CC5)n(CC5CCO5)c4c3)cc12] |
| 263 | 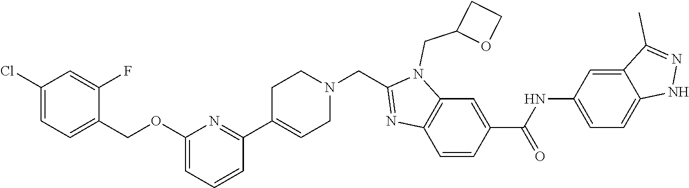 [SMILES: Cc1n[nH]c2ccc(NC(=O)c3ccc4nc(CN5CC=C(c6cccc(OCc7ccc(Cl)cc7F)n6)CC5)n(CC5CCO5)c4c3)cc12] |
| 264 | 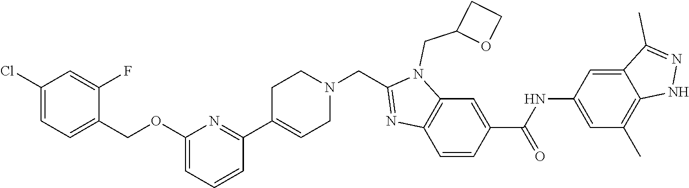 [SMILES: Cc1n[nH]c2c(C)cc(NC(=O)c3ccc4nc(CN5CC=C(c6cccc(OCc7ccc(Cl)cc7F)n6)CC5)n(CC5CCO5)c4c3)cc12] |
| 265 | 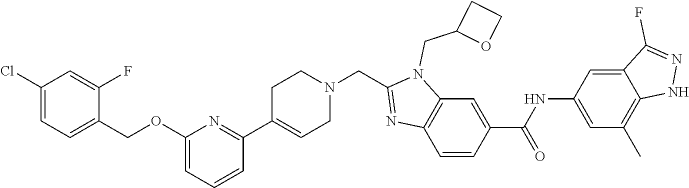 [SMILES: Cc1cc(NC(=O)c2ccc3nc(CN4CC=C(c5cccc(OCc6ccc(Cl)cc6F)n5)CC4)n(CC4CCO4)c3c2)cc2c(F)n[nH]c12] |
| 266 | 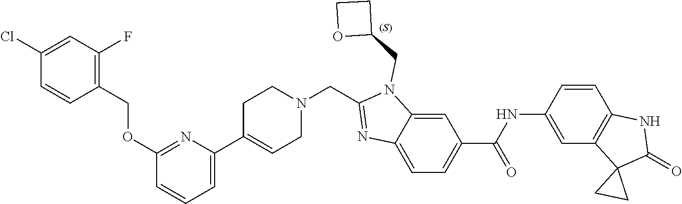 |
| 267 | 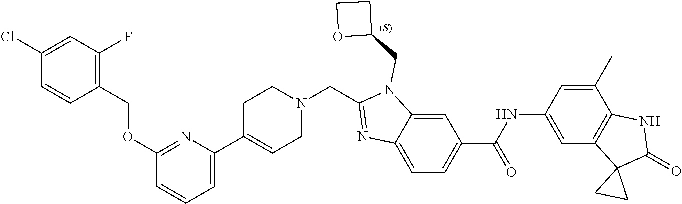 |
| 268 | 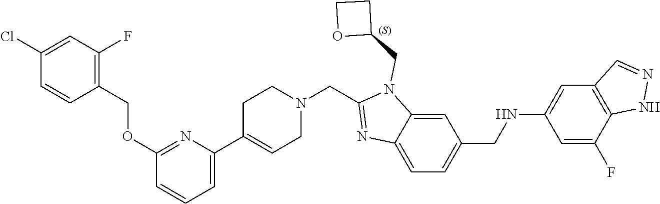 |
| 269 | 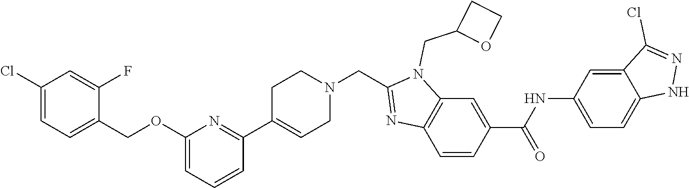 [SMILES: O=C(Nc1ccc2[nH]nc(Cl)c2c1)c1ccc2nc(CN3CC=C(c4cccc(OCc5ccc(Cl)cc5F)n4)CC3)n(CC3CCO3)c2c1] |
| 270 | 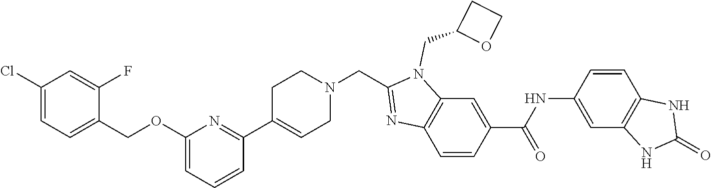 [SMILES: O=C(Nc1ccc2[nH]c(=O)[nH]c2c1)c1ccc2nc(CN3CC=C(c4cccc(OCc5ccc(Cl)cc5F)n4)CC3)n(C[C@@H]3CCO3)c2c1] |
| 271 | 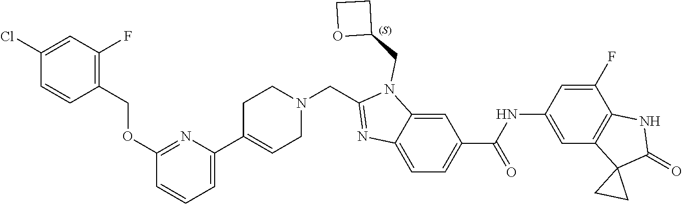 |
| 272 | 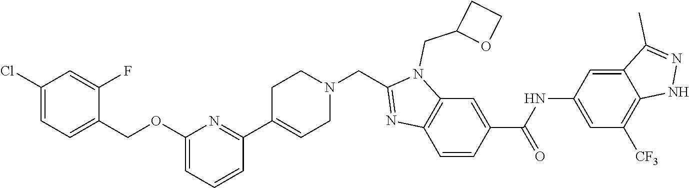 [SMILES: Cc1n[nH]c2c(C(F)(F)F)cc(NC(=O)c3ccc4nc(CN5CC=C(c6cccc(OCc7ccc(Cl)cc7F)n6)CC5)n(CC5CCO5)c4c3)cc12] |
| 273 | 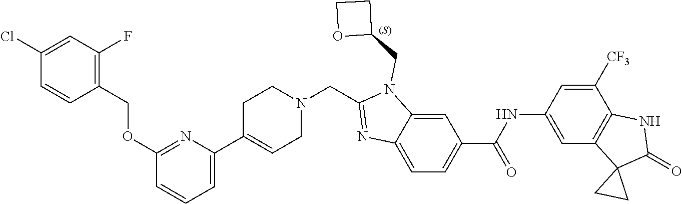 |
| 274 | 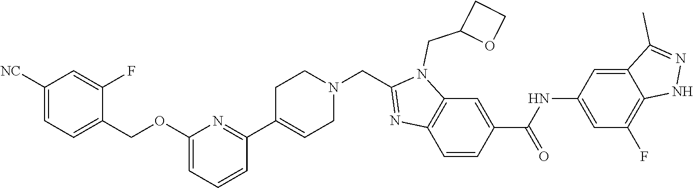 [SMILES: Cc1n[nH]c2c(F)cc(NC(=O)c3ccc4nc(CN5CC=C(c6cccc(OCc7ccc(C#N)cc7F)n6)CC5)n(CC5CCO5)c4c3)cc12] |
| 275 | 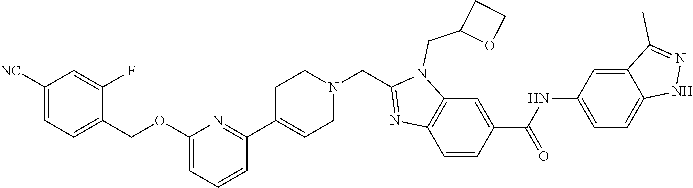 [SMILES: Cc1n[nH]c2ccc(NC(=O)c3ccc4nc(CN5CC=C(c6cccc(OCc7ccc(C#N)cc7F)n6)CC5)n(CC5CCO5)c4c3)cc12] |
| 276 | 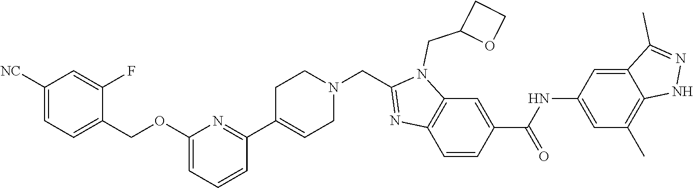 [SMILES: Cc1n[nH]c2c(C)cc(NC(=O)c3ccc4nc(CN5CC=C(c6cccc(OCc7ccc(C#N)cc7F)n6)CC5)n(CC5CCO5)c4c3)cc12] |
| 277 | 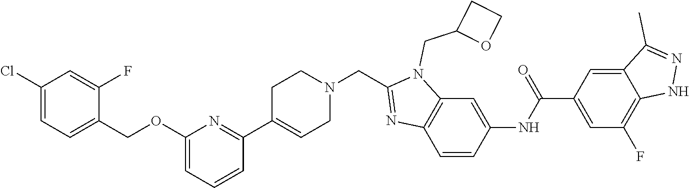 [SMILES: Cc1n[nH]c2c(F)cc(C(=O)Nc3ccc4nc(CN5CC=C(c6cccc(OCc7ccc(Cl)cc7F)n6)CC5)n(CC5CCO5)c4c3)cc12] |
| 278 | 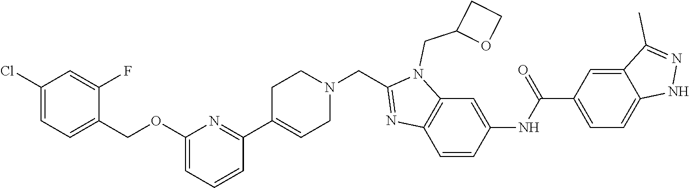 [SMILES: Cc1n[nH]c2ccc(C(=O)Nc3ccc4nc(CN5CC=C(c6cccc(OCc7ccc(Cl)cc7F)n6)CC5)n(CC5CCO5)c4c3)cc12] |
| 279 | 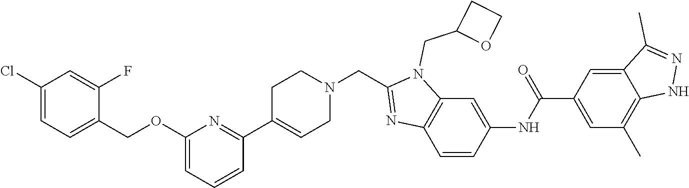 [SMILES: Cc1n[nH]c2c(C)cc(C(=O)Nc3ccc4nc(CN5CC=C(c6cccc(OCc7ccc(Cl)cc7F)n6)CC5)n(CC5CCO5)c4c3)cc12] |
| 280 | 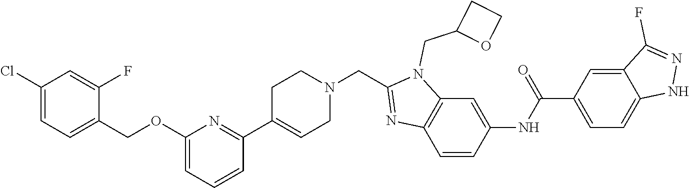 [SMILES: O=C(Nc1ccc2nc(CN3CC=C(c4cccc(OCc5ccc(Cl)cc5F)n4)CC3)n(CC3CCO3)c2c1)c1ccc2[nH]nc(F)c2c1] |
| 281 | 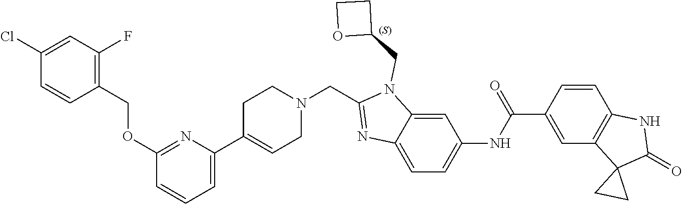 |
| 282 | 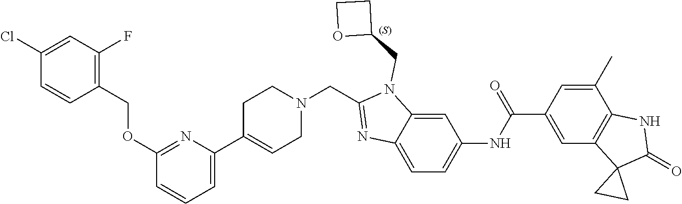 |
| 283 | 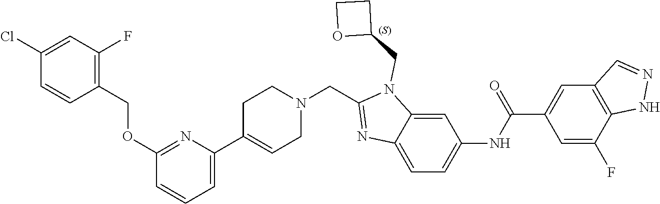 |
| 284 | 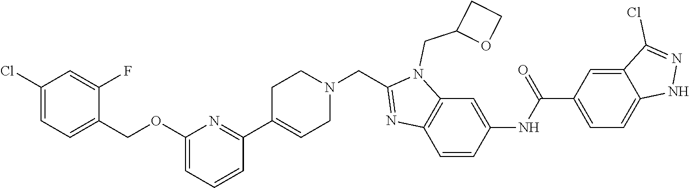 [SMILES: O=C(Nc1ccc2nc(CN3CC=C(c4cccc(OCc5ccc(Cl)cc5F)n4)CC3)n(CC3CCO3)c2c1)c1ccc2[nH]nc(Cl)c2c1] |
| 285 | 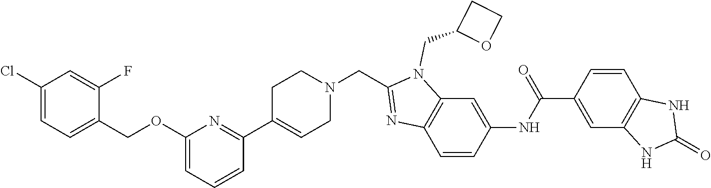 [SMILES: O=C(Nc1ccc2nc(CN3CC=C(c4cccc(OCc5ccc(Cl)cc5F)n4)CC3)n(C[C@@H]3CCO3)c2c1)c1ccc2[nH]c(=O)[nH]c2c1] |
| 286 | 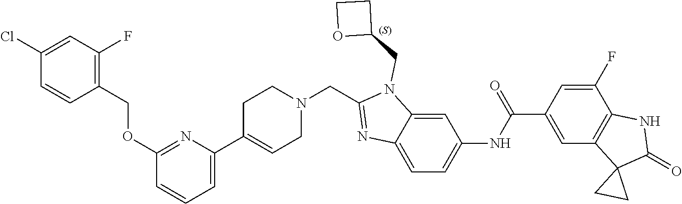 |
| 287 | 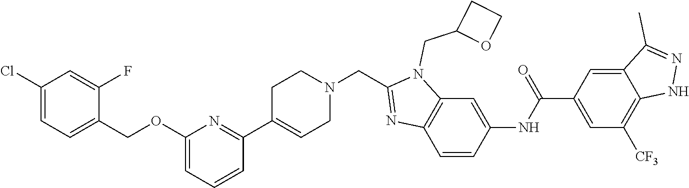 [SMILES: Cc1n[nH]c2c(C(F)(F)F)cc(C(=O)Nc3ccc4nc(CN5CC=C(c6cccc(OCc7ccc(Cl)cc7F)n6)CC5)n(CC5CCO5)c4c3)cc12] |
| 288 | 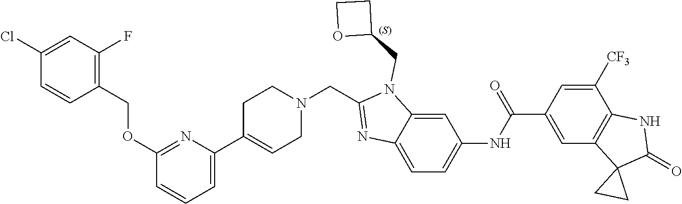 |
| 289 | 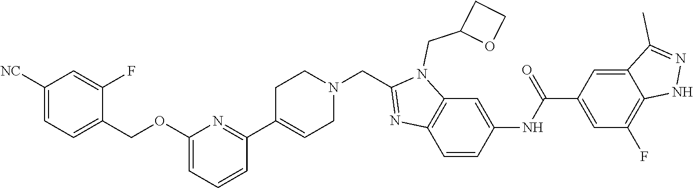 [SMILES: Cc1n[nH]c2c(F)cc(C(=O)Nc3ccc4nc(CN5CC=C(c6cccc(OCc7ccc(C#N)cc7F)n6)CC5)n(CC5CCO5)c4c3)cc12] |
| 290 | 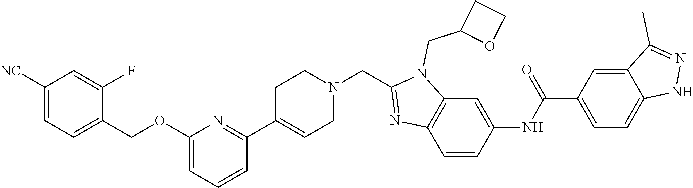 [SMILES: Cc1n[nH]c2ccc(C(=O)Nc3ccc4nc(CN5CC=C(c6cccc(OCc7ccc(C#N)cc7F)n6)CC5)n(CC5CCO5)c4c3)cc12] |
| 291 | 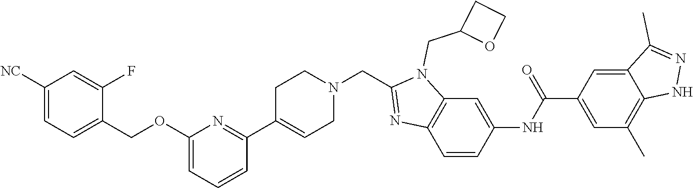 [SMILES: Cc1n[nH]c2c(C)cc(C(=O)Nc3ccc4nc(CN5CC=C(c6cccc(OCc7ccc(C#N)cc7F)n6)CC5)n(CC5CCO5)c4c3)cc12] |
| 292 | 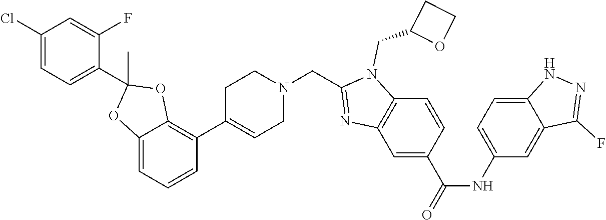 |
| 293 | 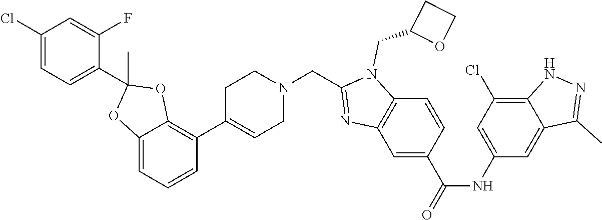 |
| 294 | 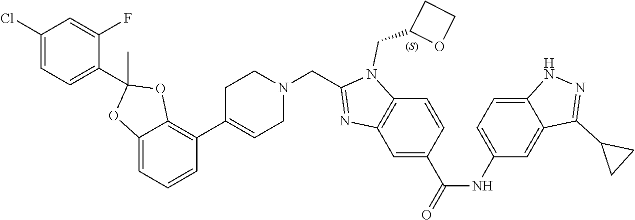 |
| 295 | 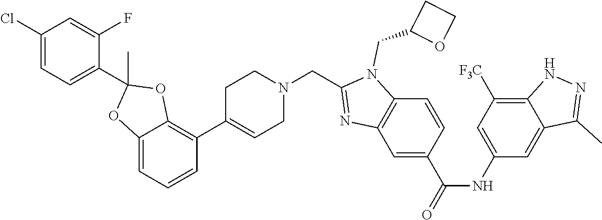 |
| 296 | 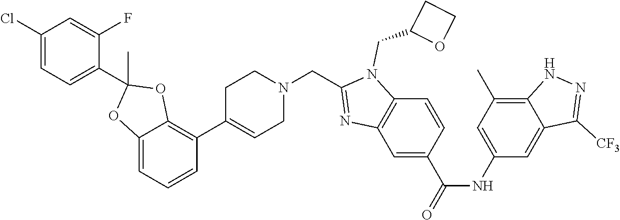 |
| 297 | 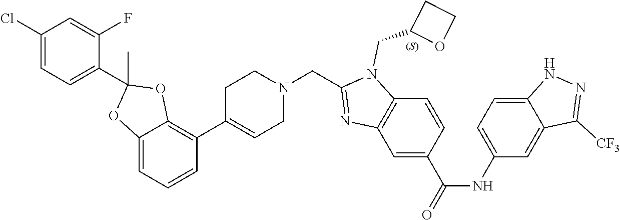 |
| 298 | 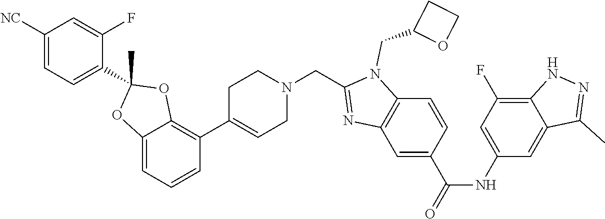 |
| 299 | 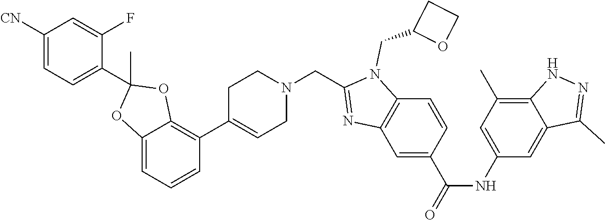 |
| 300 | 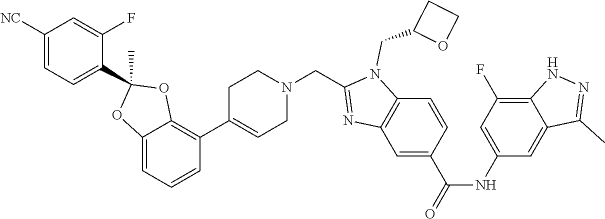 |
| 301 | 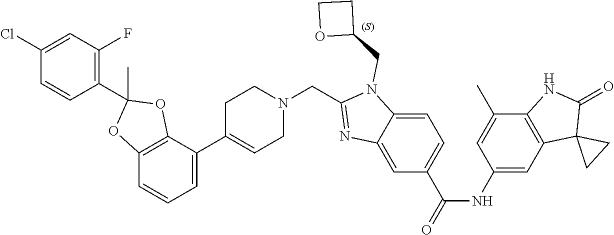 |
| 302 | 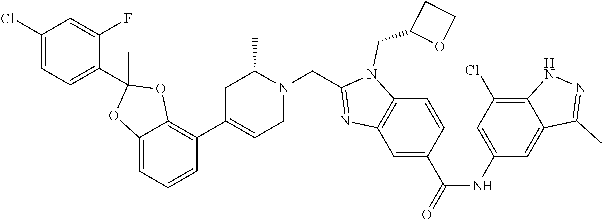 |
| 303 | 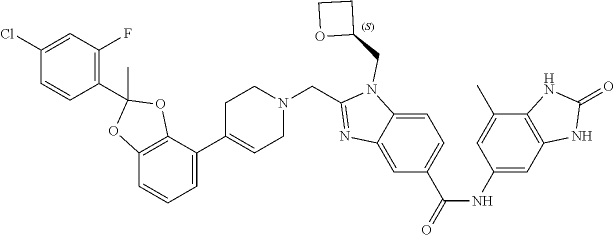 |
| 304 | 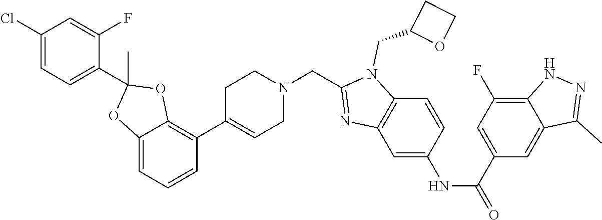 |
| 305 | 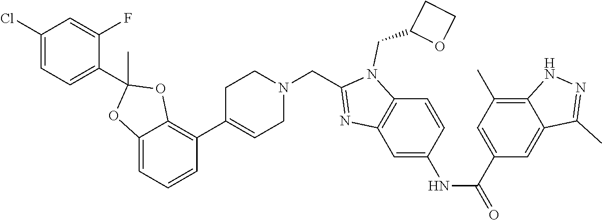 |
| 306 | 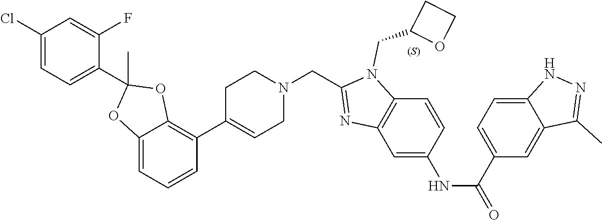 |
| 307 | 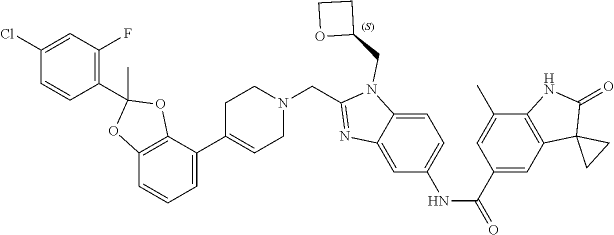 |
| 308 | 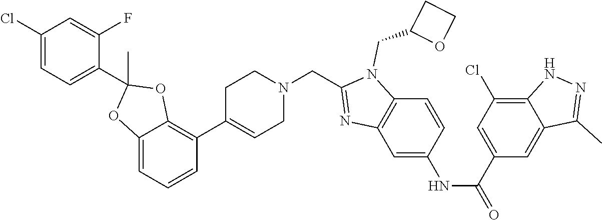 |
| 309 | 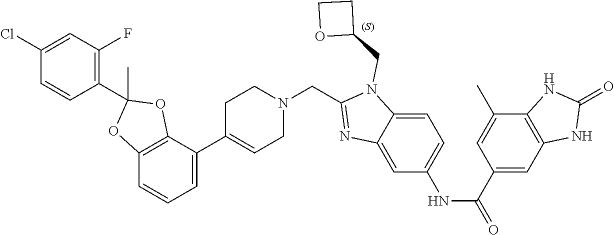 |
| 310 | 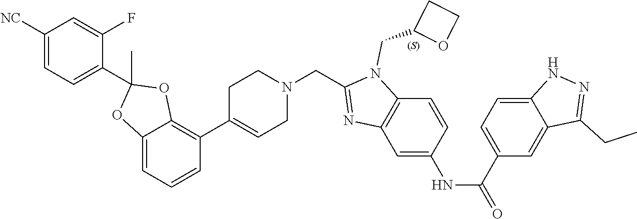 |
| 311 | 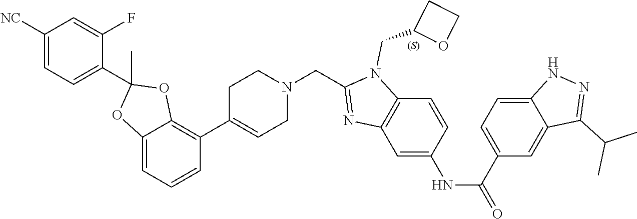 |
| 312 | 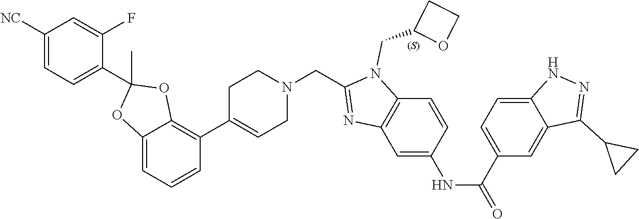 |
| 313 | 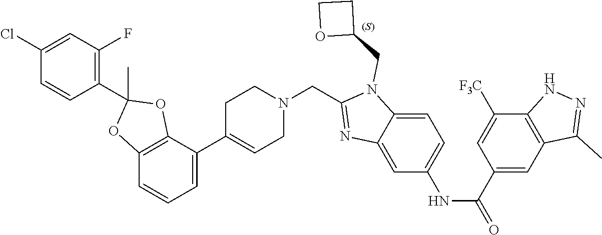 |
| 314 | 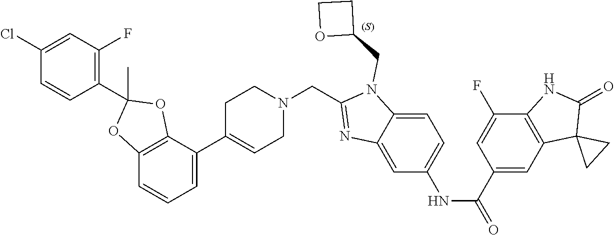 |
| 315 | 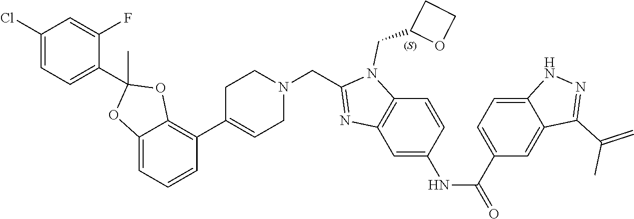 |
| 316 | 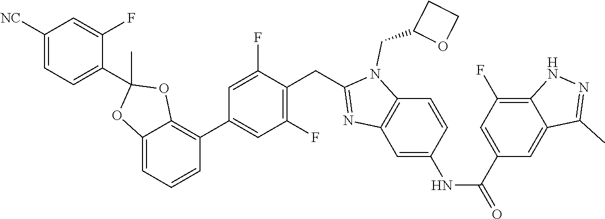 |
| 317 | 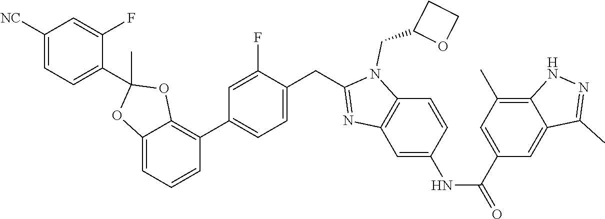 |
| 318 | 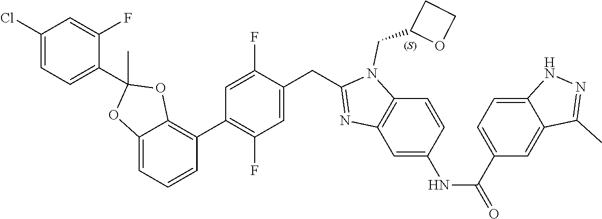 |
| 319 | 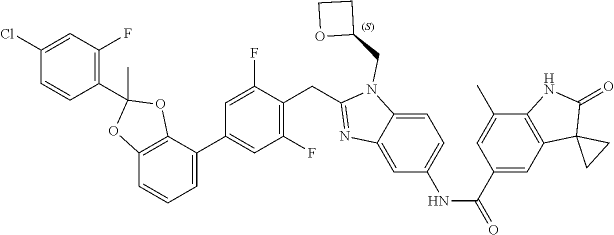 |
| 320 | 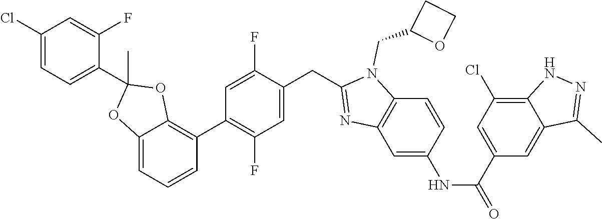 |
| 321 | 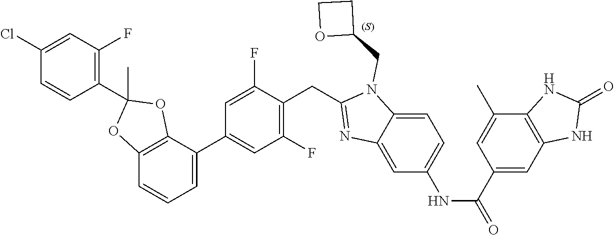 |
| 322 | 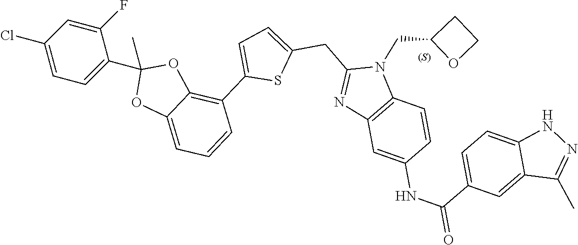 |
| 323 | 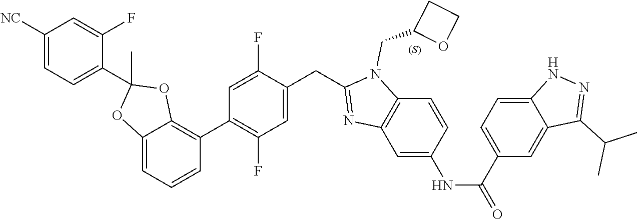 |
| 324 | 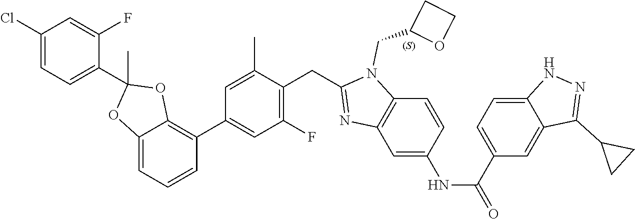 |
| 325 | 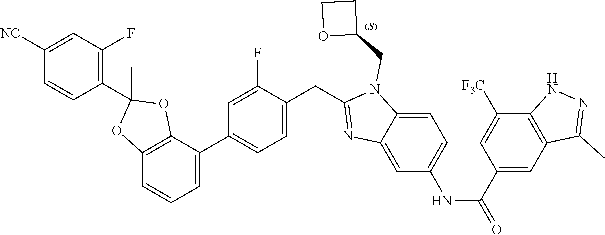 |
| 326 | 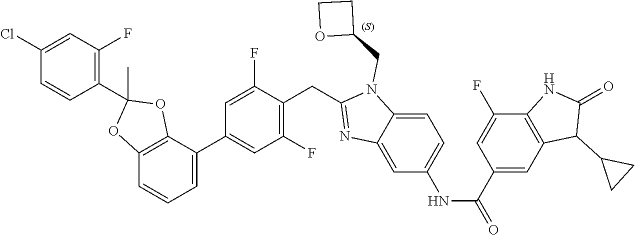 |
| 327 | 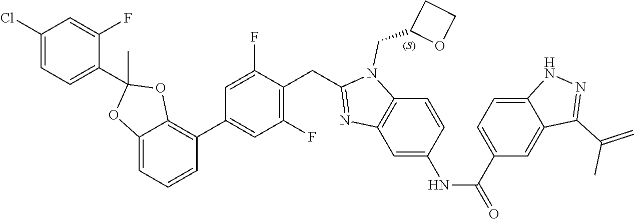 |
| 328 | 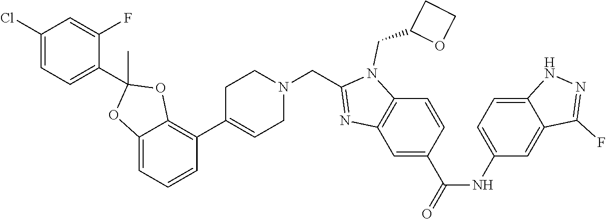 |
| 329 | 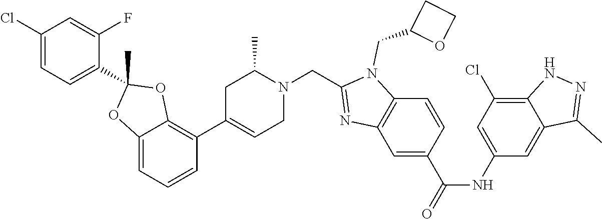 |
| 330 | 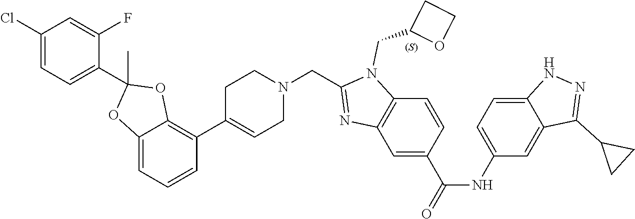 |
| 331 | 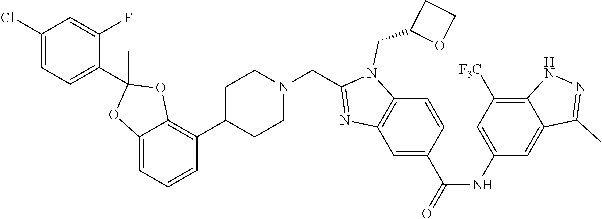 |
| 332 | 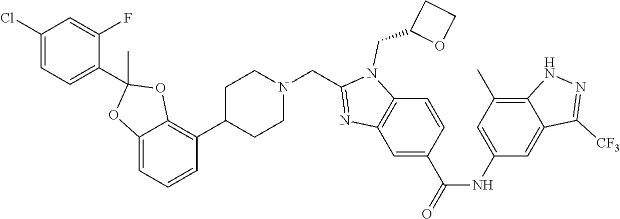 |
| 333 | 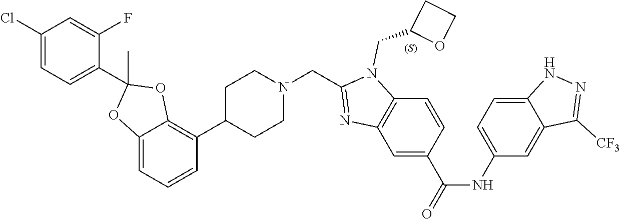 |
| 334 | 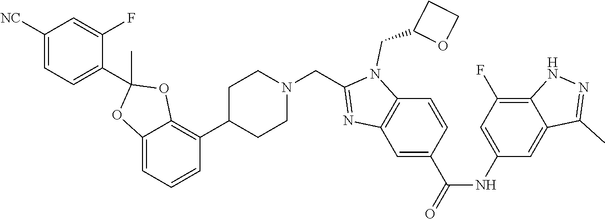 |
| 335 | 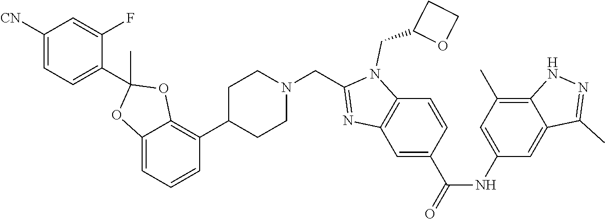 |
| 336 | 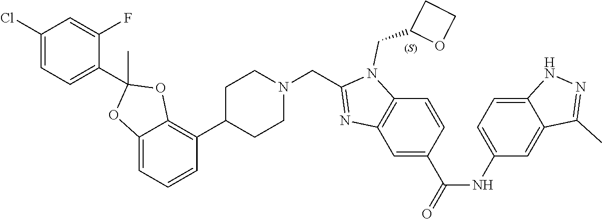 |
| 337 | 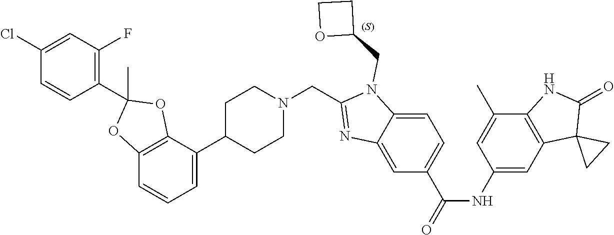 |
| 338 | 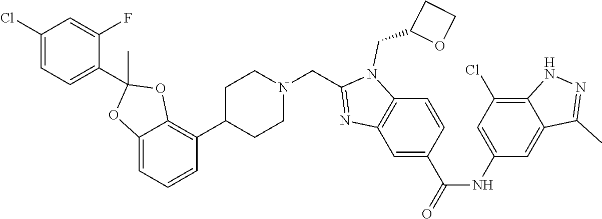 |
| 339 | 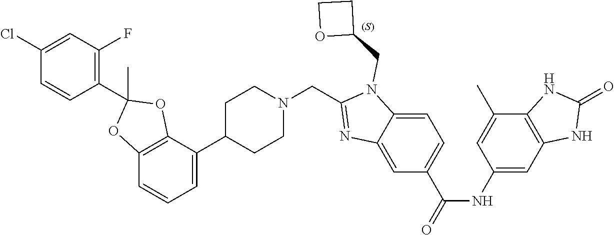 |
| 340 | 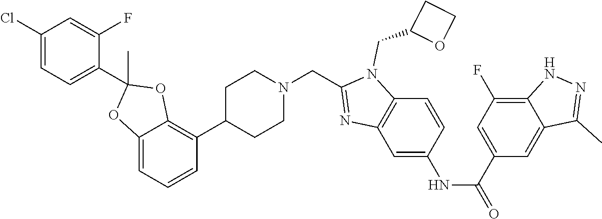 |
| 341 | 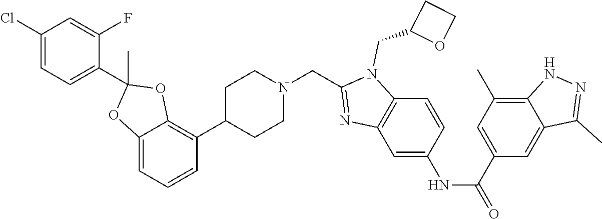 |
| 342 | 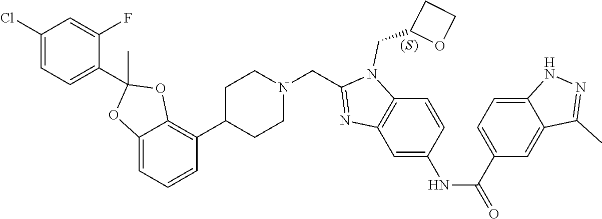 |
| 343 | 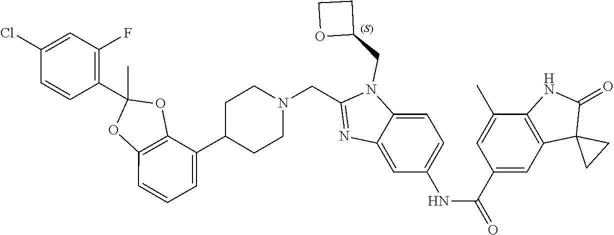 |
| 344 | 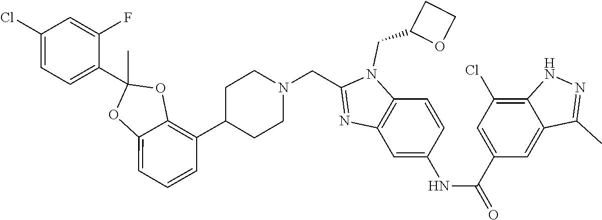 |
| 345 | 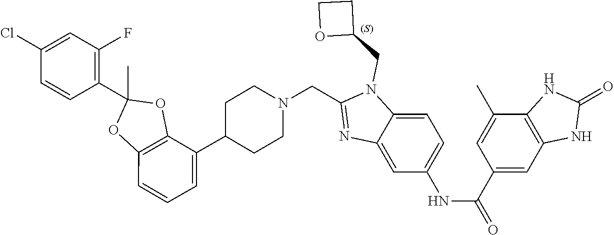 |
| 346 | 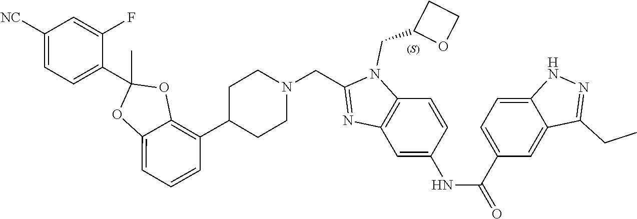 |
| 347 | 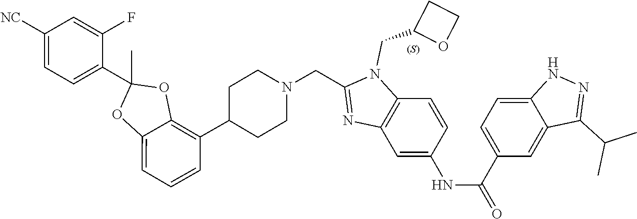 |
| 348 | 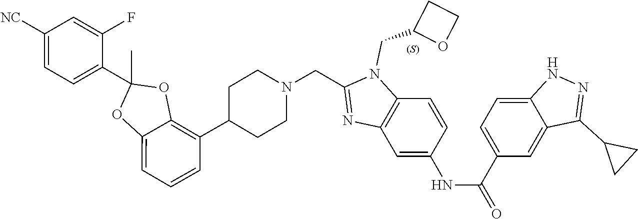 |
| 349 | 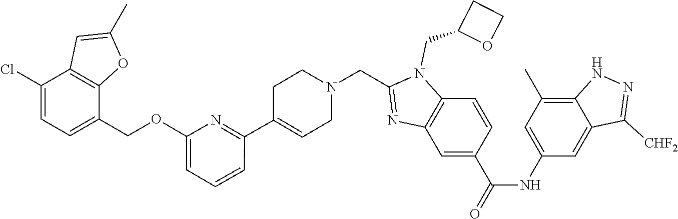 [SMILES: Cc1cc2c(Cl)ccc(COc3cccc(C4=CCN(Cc5nc6cc(C(=O)Nc7cc(C)c8[nH]nc(C(F)F)c8c7)ccc6n5C[C@@H]5CCO5)CC4)n3)c2o1] |
| 350 | 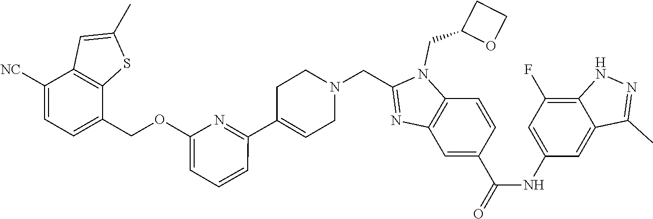 [SMILES: Cc1cc2c(C#N)ccc(COc3cccc(C4=CCN(Cc5nc6cc(C(=O)Nc7cc(F)c8[nH]nc(C)c8c7)ccc6n5C[C@@H]5CCO5)CC4)n3)c2s1] |
| 351 | 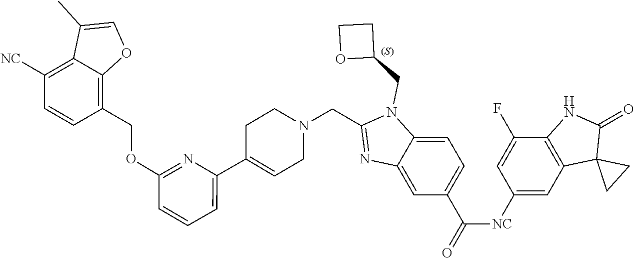 |
| 352 | 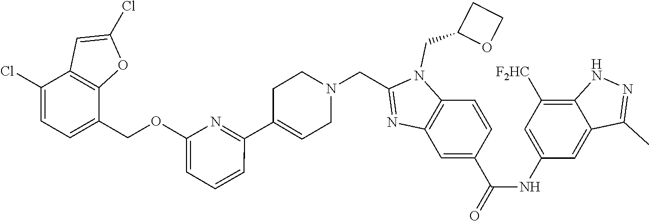 [SMILES: Cc1n[nH]c2c(C(F)F)cc(NC(=O)c3ccc4c(c3)nc(CN3CC=C(c5cccc(OCc6ccc(Cl)c7cc(Cl)oc67)n5)CC3)n4C[C@@H]3CCO3)cc12] |
| 353 | 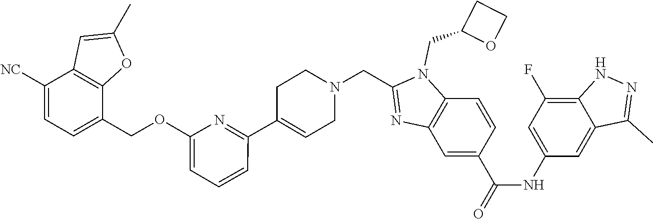 [SMILES: Cc1cc2c(C#N)ccc(COc3cccc(C4=CCN(Cc5nc6cc(C(=O)Nc7cc(F)c8[nH]nc(C)c8c7)ccc6n5C[C@@H]5CCO5)CC4)n3)c2o1] |
| 354 | 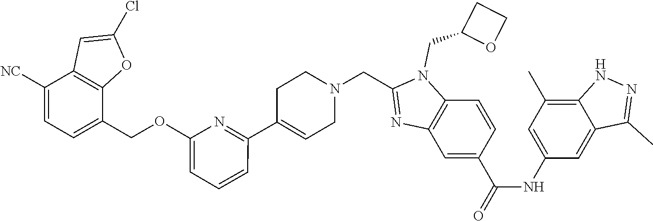 [SMILES: Cc1n[nH]c2c(C)cc(NC(=O)c3ccc4c(c3)nc(CN3CC=C(c5cccc(OCc6ccc(C#N)c7cc(Cl)oc67)n5)CC3)n4C[C@@H]3CCO3)cc12] |
| 355 | 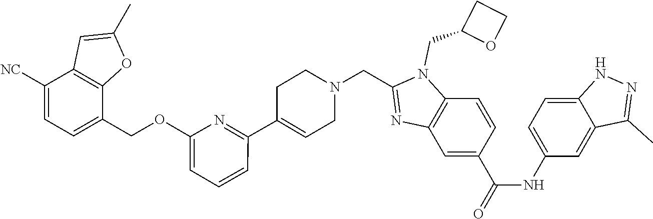 [SMILES: Cc1cc2c(C#N)ccc(COc3cccc(C4=CCN(Cc5nc6cc(C(=O)Nc7ccc8[nH]nc(C)c8c7)ccc6n5C[C@@H]5CCO5)CC4)n3)c2o1] |
| 356 | 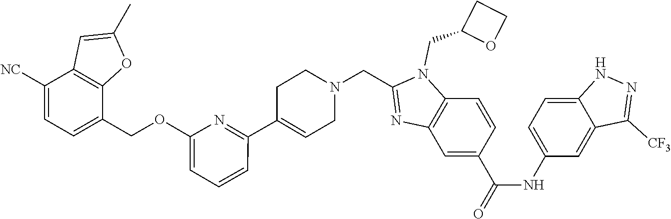 [SMILES: Cc1cc2c(C#N)ccc(COc3cccc(C4=CCN(Cc5nc6cc(C(=O)Nc7ccc8[nH]nc(C(F)(F)F)c8c7)ccc6n5C[C@@H]5CCO5)CC4)n3)c2o1] |
| 357 | 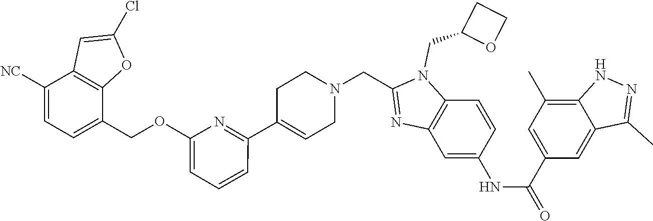 [SMILES: Cc1n[nH]c2c(C)cc(C(=O)Nc3ccc4c(c3)nc(CN3CC=C(c5cccc(OCc6ccc(C#N)c7cc(Cl)oc67)n5)CC3)n4C[C@@H]3CCO3)cc12] |
| 358 | 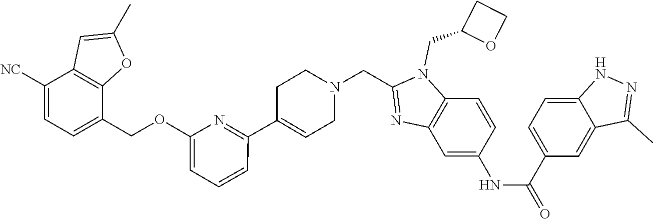 [SMILES: Cc1cc2c(C#N)ccc(COc3cccc(C4=CCN(Cc5nc6cc(NC(=O)c7ccc8[nH]nc(C)c8c7)ccc6n5C[C@@H]5CCO5)CC4)n3)c2o1] |
| 359 | 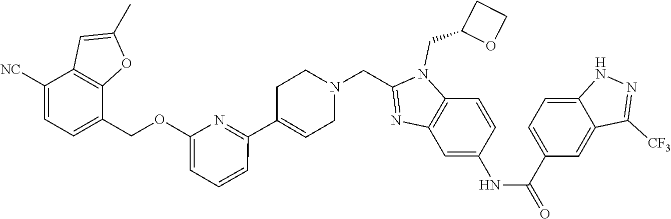 [SMILES: Cc1cc2c(C#N)ccc(COc3cccc(C4=CCN(Cc5nc6cc(NC(=O)c7ccc8[nH]nc(C(F)(F)F)c8c7)ccc6n5C[C@@H]5CCO5)CC4)n3)c2o1] |
| 360 | 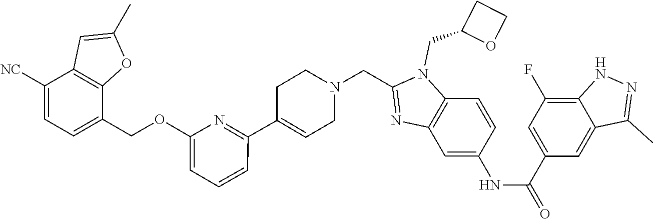 [SMILES: Cc1cc2c(C#N)ccc(COc3cccc(C4=CCN(Cc5nc6cc(NC(=O)c7cc(F)c8[nH]nc(C)c8c7)ccc6n5C[C@@H]5CCO5)CC4)n3)c2o1] |
| 361 | 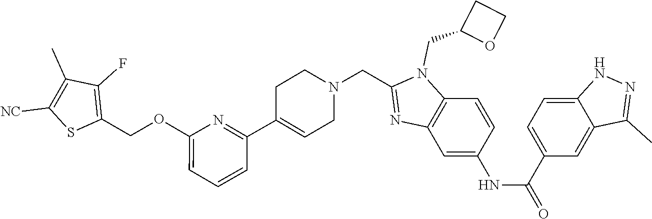 [SMILES: Cc1c(C#N)sc(COc2cccc(C3=CCN(Cc4nc5cc(NC(=O)c6ccc7[nH]nc(C)c7c6)ccc5n4C[C@@H]4CCO4)CC3)n2)c1F] |
| 362 | 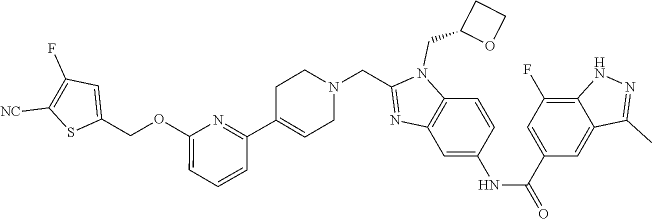 [SMILES: Cc1n[nH]c2c(F)cc(C(=O)Nc3ccc4c(c3)nc(CN3CC=C(c5cccc(OCc6cc(F)c(C#N)s6)n5)CC3)n4C[C@@H]3CCO3)cc12] |
| 363 | 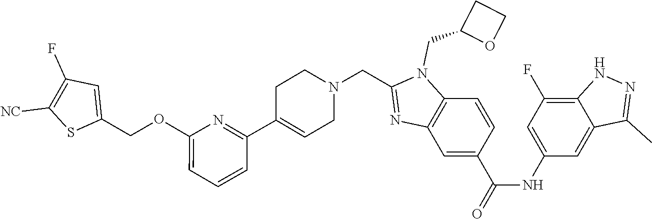 [SMILES: Cc1n[nH]c2c(F)cc(NC(=O)c3ccc4c(c3)nc(CN3CC=C(c5cccc(OCc6cc(F)c(C#N)s6)n5)CC3)n4C[C@@H]3CCO3)cc12] |
| 364 | 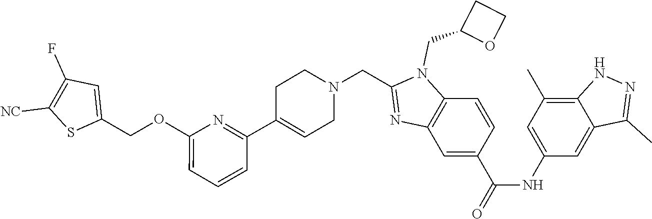 [SMILES: Cc1n[nH]c2c(C)cc(NC(=O)c3ccc4c(c3)nc(CN3CC=C(c5cccc(OCc6cc(F)c(C#N)s6)n5)CC3)n4C[C@@H]3CCO3)cc12] |
| 365 | 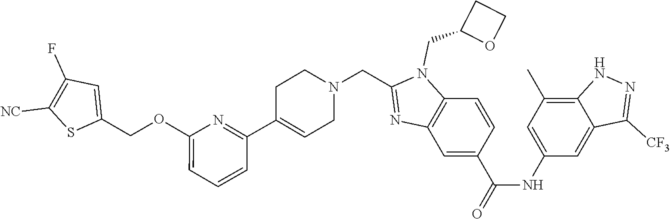 [SMILES: Cc1cc(NC(=O)c2ccc3c(c2)nc(CN2CC=C(c4cccc(OCc5cc(F)c(C#N)s5)n4)CC2)n3C[C@@H]2CCO2)cc2c(C(F)(F)F)n[nH]c12] |
| 366 | 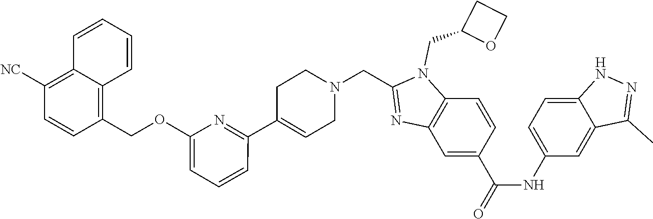 [SMILES: Cc1n[nH]c2ccc(NC(=O)c3ccc4c(c3)nc(CN3CC=C(c5cccc(OCc6ccc(C#N)c7ccccc67)n5)CC3)n4C[C@@H]3CCO3)cc12] |
| 367 | 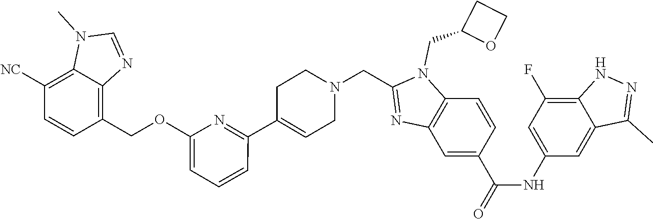 [SMILES: Cc1n[nH]c2c(F)cc(NC(=O)c3ccc4c(c3)nc(CN3CC=C(c5cccc(OCc6ccc(C#N)c7c6ncn7C)n5)CC3)n4C[C@@H]3CCO3)cc12] |
| 368 | 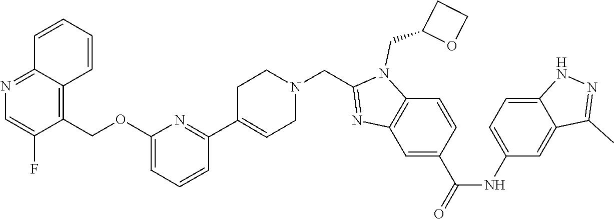 [SMILES: Cc1n[nH]c2ccc(NC(=O)c3ccc4c(c3)nc(CN3CC=C(c5cccc(OCc6c(F)cnc7ccccc67)n5)CC3)n4C[C@@H]3CCO3)cc12] |
| 369 | 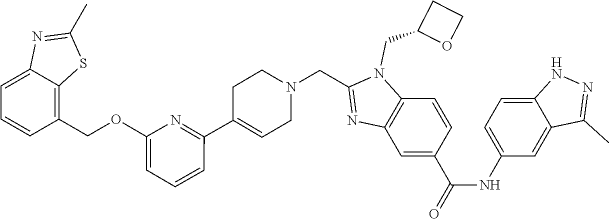 [SMILES: Cc1nc2cccc(COc3cccc(C4=CCN(Cc5nc6cc(C(=O)Nc7ccc8[nH]nc(C)c8c7)ccc6n5C[C@@H]5CCO5)CC4)n3)c2s1] |
| 370 | 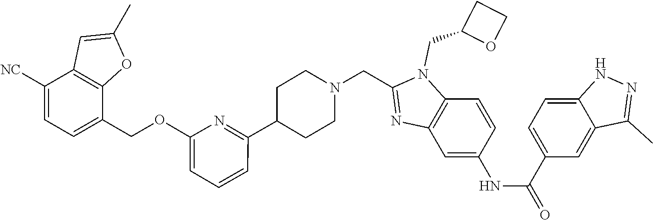 [SMILES: Cc1cc2c(C#N)ccc(COc3cccc(C4CCN(Cc5nc6cc(NC(=O)c7ccc8[nH]nc(C)c8c7)ccc6n5C[C@@H]5CCO5)CC4)n3)c2o1] |
| 371 | 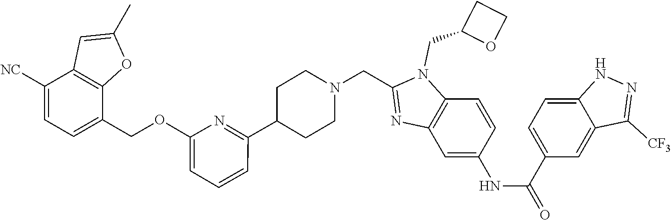 [SMILES: Cc1cc2c(C#N)ccc(COc3cccc(C4CCN(Cc5nc6cc(NC(=O)c7ccc8[nH]nc(C(F)(F)F)c8c7)ccc6n5C[C@@H]5CCO5)CC4)n3)c2o1] |
| 372 | 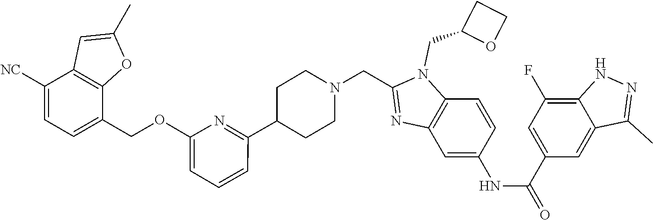 [SMILES: Cc1cc2c(C#N)ccc(COc3cccc(C4CCN(Cc5nc6cc(NC(=O)c7cc(F)c8[nH]nc(C)c8c7)ccc6n5C[C@@H]5CCO5)CC4)n3)c2o1] |
| 373 | 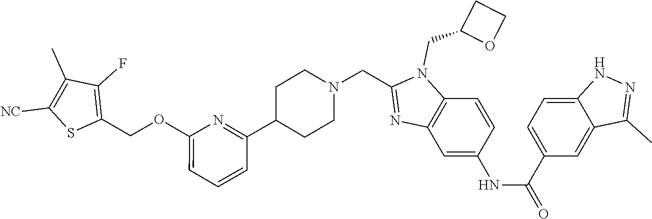 [SMILES: Cc1c(C#N)sc(COc2cccc(C3CCN(Cc4nc5cc(NC(=O)c6ccc7[nH]nc(C)c7c6)ccc5n4C[C@@H]4CCO4)CC3)n2)c1F] |
| 374 | 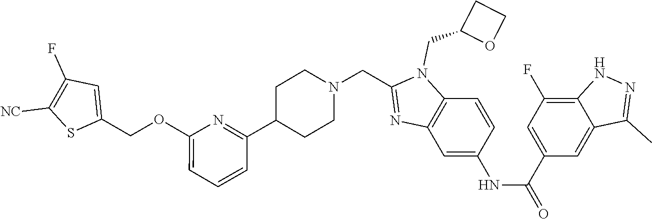 [SMILES: Cc1n[nH]c2c(F)cc(C(=O)Nc3ccc4c(c3)nc(CN3CCC(c5cccc(OCc6cc(F)c(C#N)s6)n5)CC3)n4C[C@@H]3CCO3)cc12] |
| 375 | 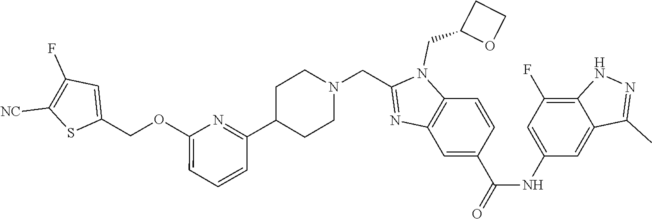 [SMILES: Cc1n[nH]c2c(F)cc(NC(=O)c3ccc4c(c3)nc(CN3CCC(c5cccc(OCc6cc(F)c(C#N)s6)n5)CC3)n4C[C@@H]3CCO3)cc12] |
| 376 | 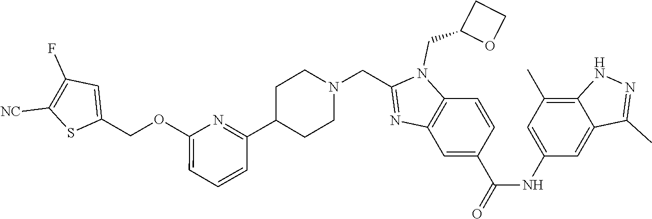 [SMILES: Cc1n[nH]c2c(C)cc(NC(=O)c3ccc4c(c3)nc(CN3CCC(c5cccc(OCc6cc(F)c(C#N)s6)n5)CC3)n4C[C@@H]3CCO3)cc12] |
| 377 | 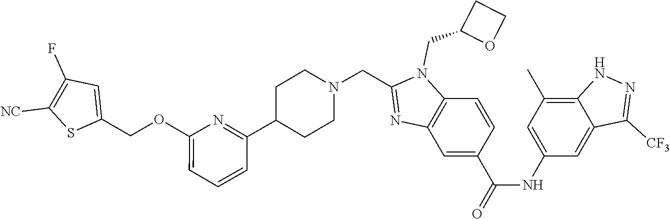 [SMILES: Cc1cc(NC(=O)c2ccc3c(c2)nc(CN2CCC(c4cccc(OCc5cc(F)c(C#N)s5)n4)CC2)n3C[C@@H]2CCO2)cc2c(C(F)(F)F)n[nH]c12] |
| 378 | 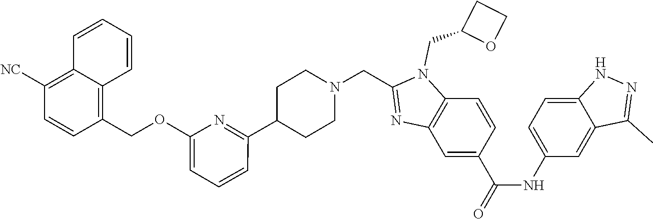 [SMILES: Cc1n[nH]c2ccc(NC(=O)c3ccc4c(c3)nc(CN3CCC(c5cccc(OCc6ccc(C#N)c7ccccc67)n5)CC3)n4C[C@@H]3CCO3)cc12] |
| 379 | 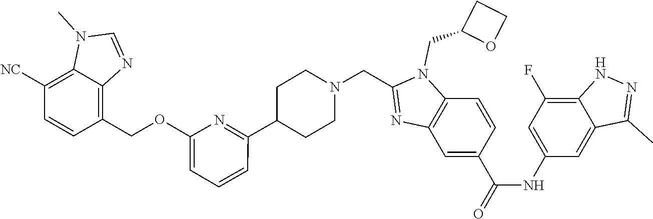 [SMILES: Cc1n[nH]c2c(F)cc(NC(=O)c3ccc4c(c3)nc(CN3CCC(c5cccc(OCc6ccc(C#N)c7c6ncn7C)n5)CC3)n4C[C@@H]3CCO3)cc12] |
| 380 | 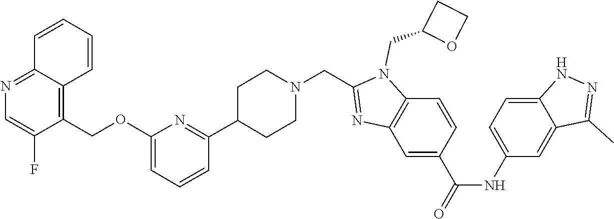 [SMILES: Cc1n[nH]c2ccc(NC(=O)c3ccc4c(c3)nc(CN3CCC(c5cccc(OCc6c(F)cnc7ccccc67)n5)CC3)n4C[C@@H]3CCO3)cc12] |
| 381 | 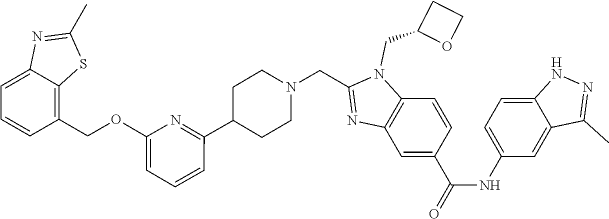 [SMILES: Cc1nc2cccc(COc3cccc(C4CCN(Cc5nc6cc(C(=O)Nc7ccc8[nH]nc(C)c8c7)ccc6n5C[C@@H]5CCO5)CC4)n3)c2s1] |
| 382 | 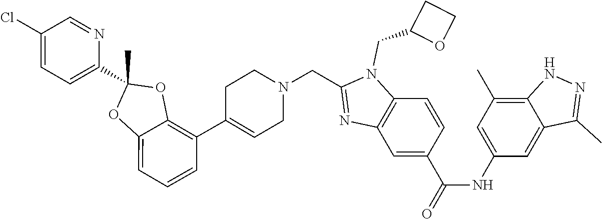 |
| 383 | 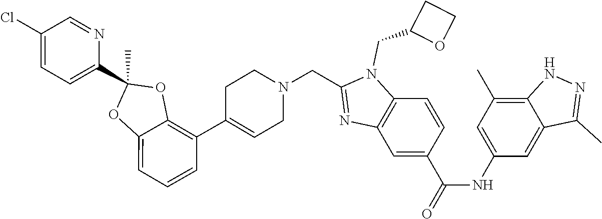 |
| 384 | 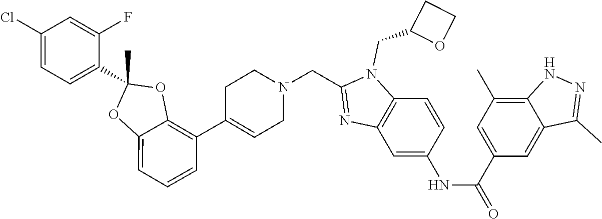 |
| 385 | 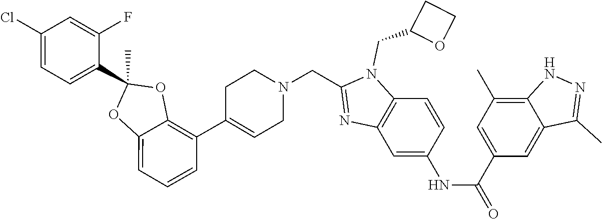 |
| 386 | 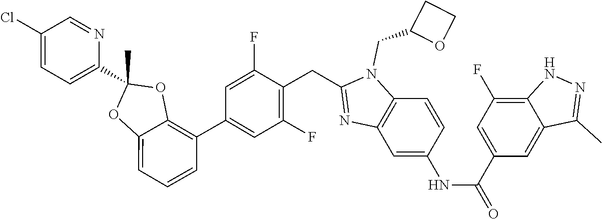 |
| 387 | 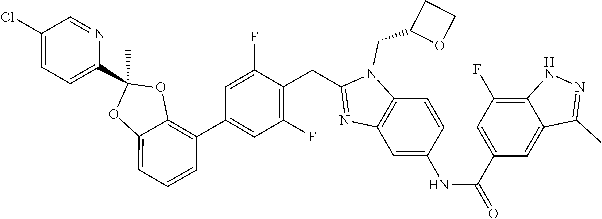 |
| 388 | 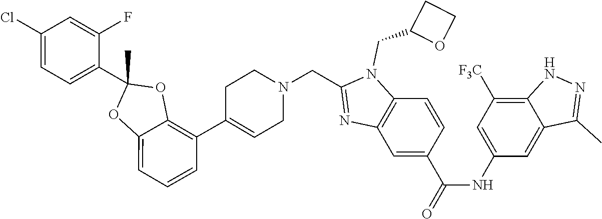 |
| 389 | 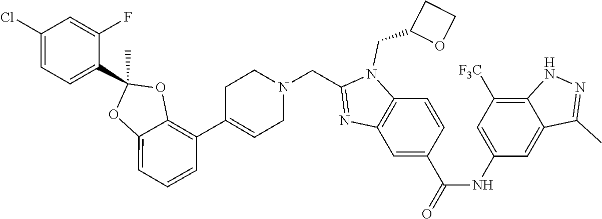 |
| 390 | 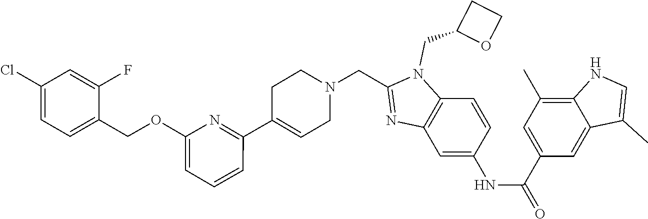 [SMILES: Cc1c[nH]c2c(C)cc(C(=O)Nc3ccc4c(c3)nc(CN3CC=C(c5cccc(OCc6ccc(Cl)cc6F)n5)CC3)n4C[C@@H]3CCO3)cc12] |
| 391 | 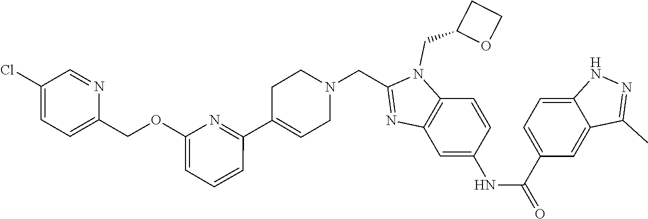 [SMILES: Cc1n[nH]c2ccc(C(=O)Nc3ccc4c(c3)nc(CN3CC=C(c5cccc(OCc6ccc(Cl)cn6)n5)CC3)n4C[C@@H]3CCO3)cc12] |
| 392 | 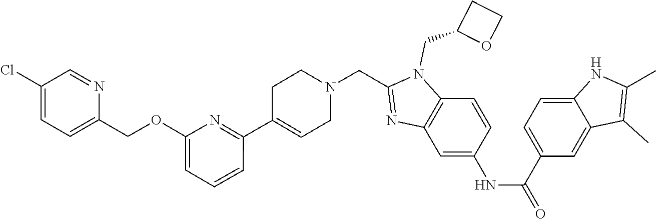 [SMILES: Cc1[nH]c2ccc(C(=O)Nc3ccc4c(c3)nc(CN3CC=C(c5cccc(OCc6ccc(Cl)cn6)n5)CC3)n4C[C@@H]3CCO3)cc2c1C] |
| 393 | 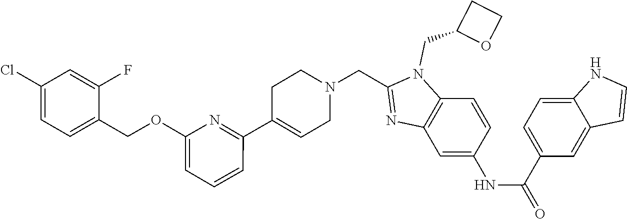 [SMILES: O=C(Nc1ccc2c(c1)nc(CN1CC=C(c3cccc(OCc4ccc(Cl)cc4F)n3)CC1)n2C[C@@H]1CCO1)c1ccc2[nH]ccc2c1] |
| 394 | 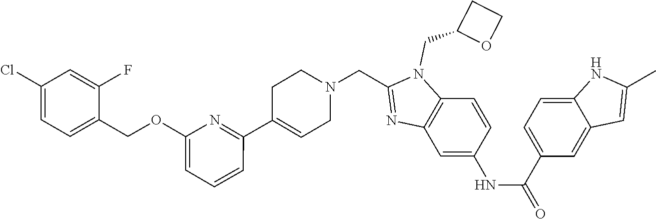 [SMILES: Cc1cc2cc(C(=O)Nc3ccc4c(c3)nc(CN3CC=C(c5cccc(OCc6ccc(Cl)cc6F)n5)CC3)n4C[C@@H]3CCO3)ccc2[nH]1] |
| 395 | 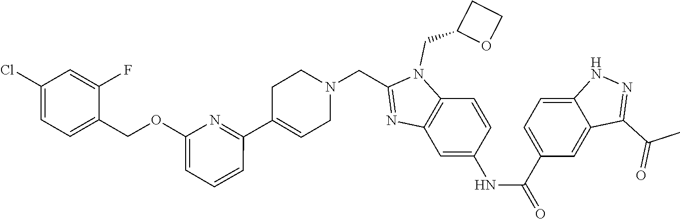 [SMILES: CC(=O)c1n[nH]c2ccc(C(=O)Nc3ccc4c(c3)nc(CN3CC=C(c5cccc(OCc6ccc(Cl)cc6F)n5)CC3)n4C[C@@H]3CCO3)cc12] |
| 396 | 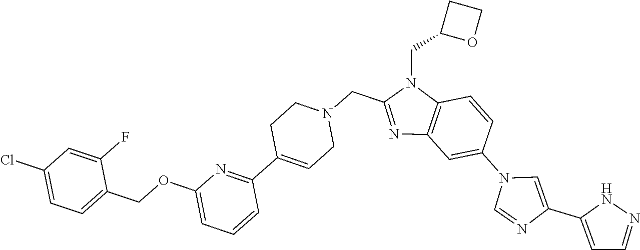 [SMILES: Fc1cc(Cl)ccc1COc1cccc(C2=CCN(Cc3nc4cc(-n5cnc(-c6ccn[nH]6)c5)ccc4n3C[C@@H]3CCO3)CC2)n1] |
| 397 | 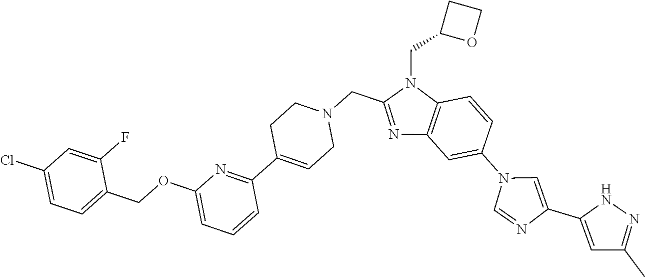 [SMILES: Cc1cc(-c2cn(-c3ccc4c(c3)nc(CN3CC=C(c5cccc(OCc6ccc(Cl)cc6F)n5)CC3)n4C[C@@H]3CCO3)cn2)[nH]n1] |
| 398 | 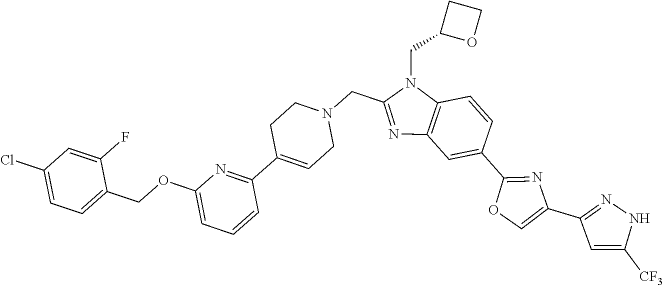 [SMILES: Fc1cc(Cl)ccc1COc1cccc(C2=CCN(Cc3nc4cc(-c5nc(-c6cc(C(F)(F)F)[nH]n6)co5)ccc4n3C[C@@H]3CCO3)CC2)n1] |
| 399 | 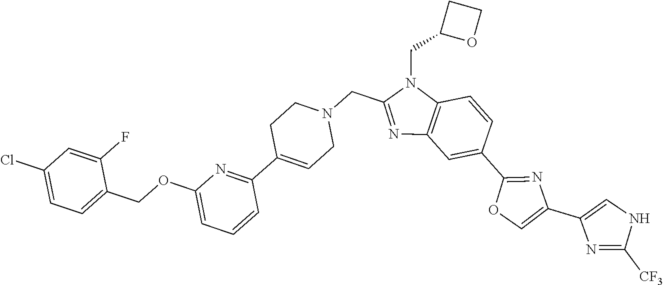 [SMILES: Fc1cc(Cl)ccc1COc1cccc(C2=CCN(Cc3nc4cc(-c5nc(-c6c[nH]c(C(F)(F)F)n6)co5)ccc4n3C[C@@H]3CCO3)CC2)n1] |
| 400 | 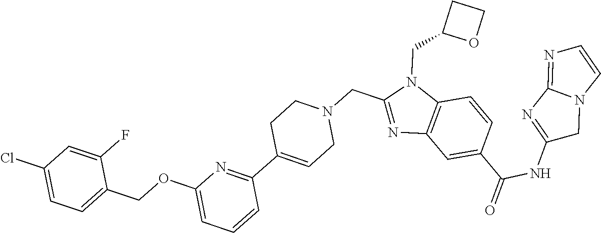 [SMILES: O=C(NC1=Nc2nccn2C1)c1ccc2c(c1)nc(CN1CC=C(c3cccc(OCc4ccc(Cl)cc4F)n3)CC1)n2C[C@@H]1CCO1] |
| 401 | 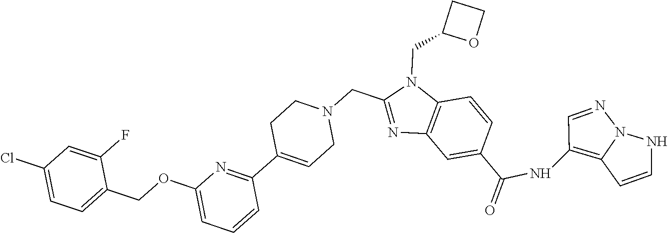 [SMILES: O=C(Nc1cnn2[nH]ccc12)c1ccc2c(c1)nc(CN1CC=C(c3cccc(OCc4ccc(Cl)cc4F)n3)CC1)n2C[C@@H]1CCO1] |
| 402 | 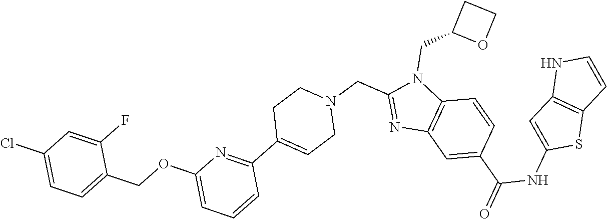 [SMILES: O=C(Nc1cc2[nH]ccc2s1)c1ccc2c(c1)nc(CN1CC=C(c3cccc(OCc4ccc(Cl)cc4F)n3)CC1)n2C[C@@H]1CCO1] |
| 403 | 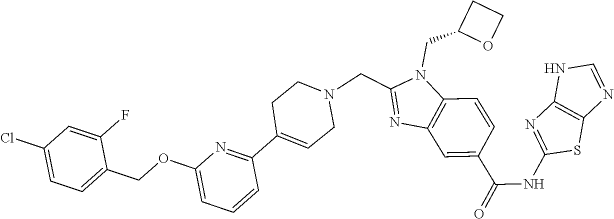 [SMILES: O=C(Nc1nc2[nH]cnc2s1)c1ccc2c(c1)nc(CN1CC=C(c3cccc(OCc4ccc(Cl)cc4F)n3)CC1)n2C[C@@H]1CCO1] |
| 404 | 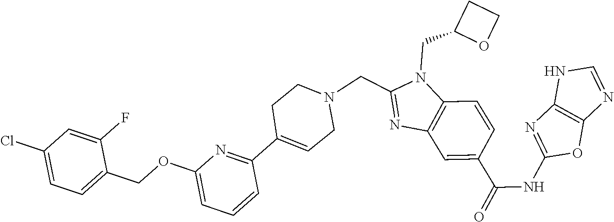 [SMILES: O=C(Nc1nc2[nH]cnc2o1)c1ccc2c(c1)nc(CN1CC=C(c3cccc(OCc4ccc(Cl)cc4F)n3)CC1)n2C[C@@H]1CCO1] |
| 405 | 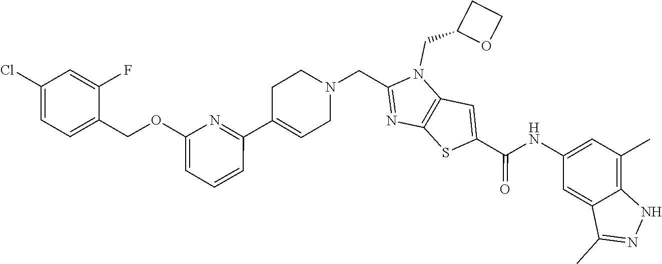 [SMILES: Cc1n[nH]c2c(C)cc(NC(=O)c3cc4c(nc(CN5CC=C(c6cccc(OCc7ccc(Cl)cc7F)n6)CC5)n4C[C@@H]4CCO4)s3)cc12] |
| 406 | 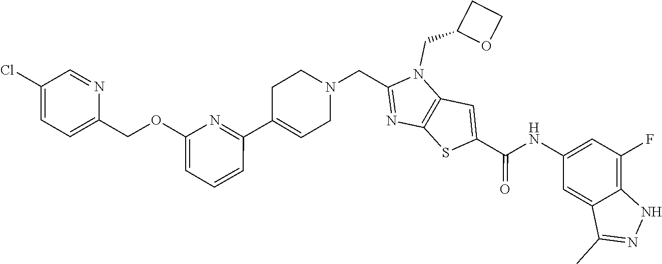 [SMILES: Cc1n[nH]c2c(F)cc(NC(=O)c3cc4c(nc(CN5CC=C(c6cccc(OCc7ccc(Cl)cn7)n6)CC5)n4C[C@@H]4CCO4)s3)cc12] |
| 407 | 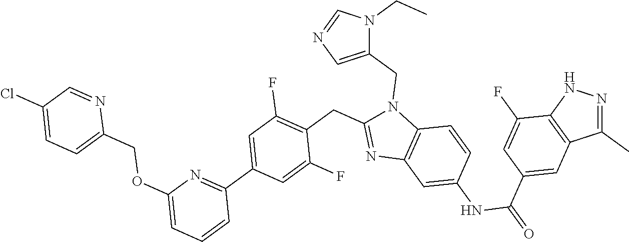 [SMILES: CCn1cncc1Cn1c(Cc2c(F)cc(-c3cccc(OCc4ccc(Cl)cn4)n3)cc2F)nc2cc(NC(=O)c3cc(F)c4[nH]nc(C)c4c3)ccc21] |
| 408 | 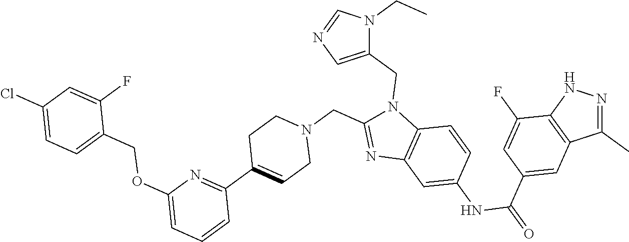 [SMILES: CCn1cncc1Cn1c(CN2CC=C(c3cccc(OCc4ccc(Cl)cc4F)n3)CC2)nc2cc(NC(=O)c3cc(F)c4[nH]nc(C)c4c3)ccc21] |
| 409 | 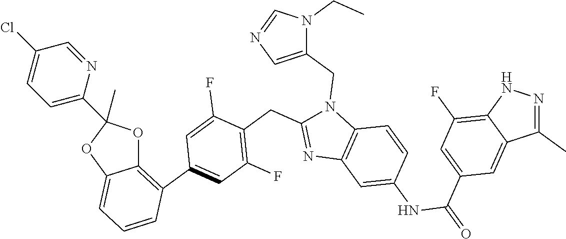 [SMILES: CCn1cncc1Cn1c(Cc2c(F)cc(-c3cccc4c3OC(C)(c3ccc(Cl)cn3)O4)cc2F)nc2cc(NC(=O)c3cc(F)c4[nH]nc(C)c4c3)ccc21] |
| 410 | 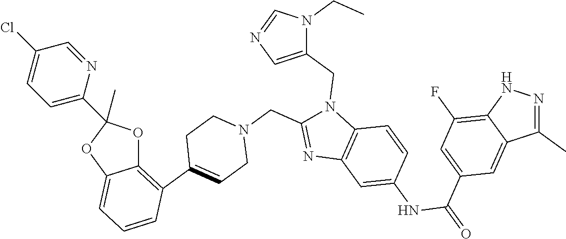 [SMILES: CCn1cncc1Cn1c(CN2CC=C(c3cccc4c3OC(C)(c3ccc(Cl)cn3)O4)CC2)nc2cc(NC(=O)c3cc(F)c4[nH]nc(C)c4c3)ccc21] |
| 411 | 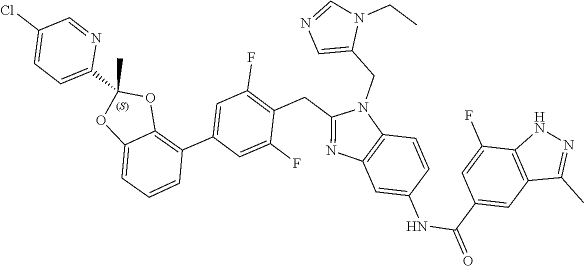 |
| 412 | 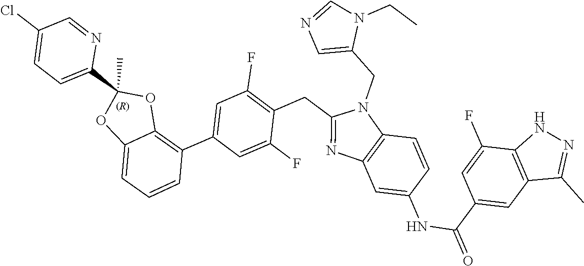 |
| 413 | 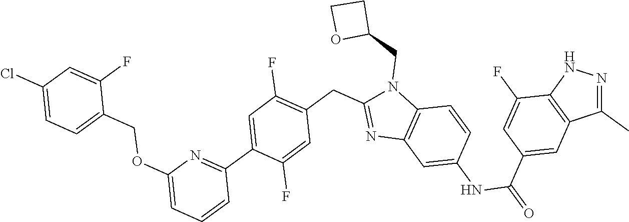 [SMILES: Cc1n[nH]c2c(F)cc(C(=O)Nc3ccc4c(c3)nc(Cc3cc(F)c(-c5cccc(OCc6ccc(Cl)cc6F)n5)cc3F)n4C[C@@H]3CCO3)cc12] |
| 414 | 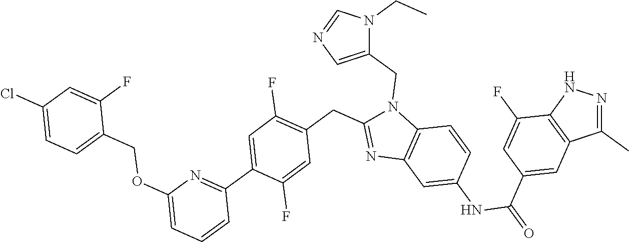 [SMILES: CCn1cncc1Cn1c(Cc2cc(F)c(-c3cccc(OCc4ccc(Cl)cc4F)n3)cc2F)nc2cc(NC(=O)c3cc(F)c4[nH]nc(C)c4c3)ccc21] |
| 415 | 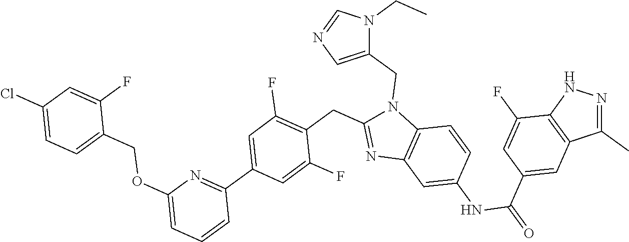 [SMILES: CCn1cncc1Cn1c(Cc2c(F)cc(-c3cccc(OCc4ccc(Cl)cc4F)n3)cc2F)nc2cc(NC(=O)c3cc(F)c4[nH]nc(C)c4c3)ccc21] |
| 416 | 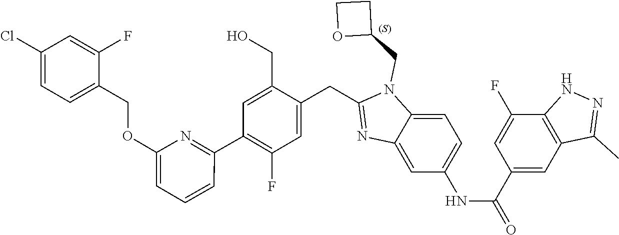 |
| 417 | 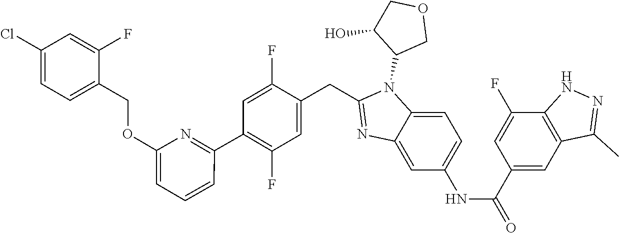 |

| | ES/MS | | |
|---|---|---|---|
| | (m/z, | | |
| No. | M + H⁺) | ¹H NMR | Procedure |
| 1 | 656.2 | ¹H NMR (500 MHz, Chloroform-d) δ | 1 |
| | | 8.19 (t, J = 4.9 Hz, 1H), 8.13-8.06 | |
| | | (m, 2H), 8.04-8.02 (m, 1H), 7.75- | |
| | | 7.73 (m, 1H), 7.66-7.63 (m, 1H), | |
| | | 7.48-7.43 (m, 3H), 7.40-7.34 (m, | |
| | | 2H), 7.28-7.21 (m, 2H), 7.02-6.96 | |
| | | (m, 2H), 6.79-6.74 (m, 1H), 6.74- | |
| | | 6.70 (m, 2H), 6.14 (s, 1H), 5.31 (s, | |
| | | 2H), 4.38-4.22 (m, 4H), 3.75-3.45 | |
| | | (m, 4H), 3.35 (s, 3H), 2.87-2.83 (m, | |
| | | 2H). | |
| 2 | 735.2 | ¹H NMR (500 MHz, Chloroform-d) δ | 2 |
| | | 8.14-8.04 (m, 2H), 7.74-7.72 (m, | |
| | | 1H), 7.65-7.35 (m, 3H), 7.21-7.18 | |
| | | (m, 1H), 7.18-7.13 (m, 2H), 6.80- | |
| | | 6.77 (m, 1H), 5.35 (s, 2H), 4.51- | |
| | | 4.32 (m, 3H), 4.32-4.24 (m, 1H), | |
| | | 4.23-4.15 (m, 1H), 4.01-3.83 (m, | |
| | | 3H), 3.78-3.65 (m, 4H), 3.61- | |
| | | 3.54 (m, 1H), 3.35-2.99 (m, 2H), 2.83- | |
| | | 2.74 (m, 2H), 2.69-2.60 (m, 2H), | |
| | | 2.42-2.30 (m, 2H), 2.25-2.09 (m, | |
| | | 7H). | |
| 3 | 692.1 | ¹H NMR (500 MHz, Chloroform-d) δ | 3 |
| | | 12.31 (s, 1H), 10.54 (s, 1H), 8.05- | |
| | | 8.02 (m, 1H), 7.88-7.87 (m, 1H), 7.86- | |
| | | 7.84 (m, 1H), 7.68-7.66 (m, 1H), | |
| | | 7.49-7.34 (m, 4H), 7.21-7.13 (m, | |
| | | 2H), 6.89-6.84 (m, 1H), 6.58-6.53 | |
| | | (m, 1H), 5.35-5.24 (m, 2H), 4.39- | |
| | | 4.24 (m, 2H), 4.23-4.15 (m, 1H), | |
| | | 4.00-3.82 (m, 4H), 3.78-3.70 (m, | |
| | | 2H), 3.36-2.97 (m, 4H), 2.90-2.84 | |
| | | (m, 5H), 2.36-2.31 (m, 1H), 2.09- | |
| | | 1.99 (m, 1H). | |
| 4 | 692.1 | ¹H NMR (500 MHz, Chloroform-d) δ | 4 |
| | | 12.01 (s, 1H), 10.64 (s, 1H), 8.07- | |
| | | 7.89 (m, 2H), 7.82-7.78 (m, 1H), | |
| | | 7.66-7.63 (m, 1H), 7.47-7.33 (m, | |
| | | 4H), 7.21-7.16 (m, 2H), 6.89-6.87 | |
| | | (m, 1H), 6.58-6.54 (m, 1H), 5.35- | |
| | | 5.24 (m, 2H), 4.39-4.13 (m, 3H), | |
| | | 4.00-3.70 (m, 6H), 3.36-3.28 (m, | |
| | | 2H), 3.08-2.90 (m, 5H), 2.93- | |
| | | 2.84 (m, 2H), 2.39-2.09 (m, 2H). | |
| 5 | 719.2 | ¹H NMR (500 MHz, Chloroform-d) δ | |
| | | 8.01-7.98 (m, 1H), 7.83-7.81 (m, | |
| | | 1H), 7.68-7.57 (m, 2H), 7.41-7.27 | |
| | | (m, 3H), 7.21-7.07 (m, 2H), 7.01- | |
| | | 6.93 (m, 2H), 6.10-6.07 (m, 1H), | |
| | | 4.39-4.13 (m, 3H), 3.97-3.70 (m, | |
| | | 4H), 3.37-3.29 (m, 5H), 2.89-2.71 | |
| | | (m, 4H), 2.36-2.31 (m, 1H), 2.09- | |
| | | 1.99 (m, 1H), 1.82 (s, 3H). | |
| 6 | 719.2 | ¹H NMR (500 MHz, Chloroform-d) δ | 5 |
| | | 8.01-7.98 (m, 1H), 7.83-7.81 (m, | |
| | | 1H), 7.68-7.57 (m, 2H), 7.41-7.27 | |
| | | (m, 3H), 7.21-7.07 (m, 2H), 7.01- | |
| | | 6.93 (m, 2H), 6.10-6.07 (m, 1H), | |
| | | 4.39-4.13 (m, 3H), 3.97-3.70 (m, | |
| | | 4H), 3.37-3.29 (m, 5H), 2.89-2.71 | |
| | | (m, 4H), 2.36-2.31 (m, 1H), 2.09- | |
| | | 1.99 (m, 1H), 1.82 (s, 3H). | |
| 7 | 685.4 | ¹H NMR (500 MHz, Chloroform-d) δ | 6 |
| | | 11.93 (s, 1H), 10.41 (s, 1H), 8.11- | |
| | | 8.10 (m, 1H), 7.88 (d, J = 2.1 Hz, | |
| | | 1H), 7.86-7.81 (m, 1H), 7.68-7.66 | |
| | | (m, 1H), 7.50-7.44 (m, 3H), 7.44- | |
| | | 7.39 (m, 1H), 7.39-7.33 (m, 1H) | |
| | | 7.33-7.26 (m, 1H), 6.57-6.23 (m, | |
| | | 2H), 5.31 (s, 2H), 4.40-3.51 (m, 8H), | |
| | | 3.35-3.20 (m, 4H), 2.90 (s, 3H), 2.12- | |
| | | 1.75 (m, 6H). | |
| 8 | 685.4 | ¹H NMR (500 MHz, Chloroform-d) δ | 6 |
| | | 12.31 (s, 1H), 10.83 (s, 1H), 8.13- | |
| | | 8.11 (m, 1H), 7.83-7.79 (m, 2H), | |
| | | 7.68-7.66 (m, 1H), 7.50-7.44 (m, | |
| | | 3H), 7.44-7.39 (m, 1H), 7.39-7.26 | |
| | | (m, 2H), 6.57-6.53 (m, 1H), 6.23- | |
| | | 6.19 (m, 1H), 5.31 (s, 2H), 4.40-3.75 | |
| | | (m, 4H), 3.64-3.51 (m, 4H), 3.35- | |
| | | 3.20 (m, 4H), 2.90 (s, 3H), 2.12- | |
| | | 1.75 (m, 6H). | |
| 9 | 655.2 | ¹H NMR (500 MHz, Chloroform-d) δ | 1 |
| | | 10.33 (s, 1H), 8.13-8.02 (m, 4H), | |
| | | 7.77-7.64 (m, 2H), 7.48-7.33 (m, | |
| | | 5H), 7.28-7.21 (m, 2H), 7.02-6.96 | |
| | | (m, 2H), 6.79-6.70 (m, 3H), 6.01- | |
| | | 5.31 (m, 4H), 4.38-3.75 (m, 6H), 3.51- | |
| | | 3.35 (m, 5H), 2.87-2.83 (m, 2H). | |
| 10 | 831.2 | ¹H NMR (500 MHz, Chloroform-d) δ | 1 |
| | | 8.22 (s, 1H), 8.15-8.12 (m, 1H), 8.11- | |
| | | 8.07 (m, 3H), 7.89-7.73 (m, 6H), | |
| | | 7.66-7.64 (m, 1H), 7.48-7.33 (m, | |
| | | 5H), 7.27-7.15 (m, 6H), 6.79-6.74 | |
| | | (m, 1H), 5.31 (s, 2H), 4.41-4.38 (m, | |
| | | 2H), 4.22 (t, J = 1.0 Hz, 2H), 3.75 (t, | |
| | | J = 4.4 Hz, 2H), 3.51-3.35 (m, 7H), | |
| | | 2.93-2.87 (m, 4H). | |
| 11 | 672.2 | ¹H NMR (500 MHz, Chloroform-d) δ | 1 |
| | | 8.18-8.15 (m, 1H), 8.13-8.06 (m, | |
| | | 2H), 8.04-8.02 (m, 1H), 7.77-7.64 | |
| | | (m, 2H), 7.48-7.35 (m, 5H), 7.28- | |
| | | 7.21 (m, 2H), 7.02-6.96 (m, 2H), | |
| | | 6.79-6.70 (m, 3H), 6.14 (s, 1H), | |
| | | 5.31 (s, 2H), 4.41-4.38 (m, 2H), 4.27 | |
| | | (s, 2H), 3.75 (t, J = 4.4 Hz, 2H), 3.51- | |
| | | 3.44 (m, 2H), 3.36 (s, 3H), 2.87- | |
| | | 2.83 (m, 2H). | |
| 12 | 684.4 | ¹H NMR (500 MHz, Chloroform-d) δ | 1 |
| | | 11.91 (s, 1H), 8.22 (s, 1H), 8.18-8.14 | |
| | | (m, 1H), 8.11-8.07 (m, 3H), 7.77- | |
| | | 7.64 (m, 2H), 7.48-7.35 (m, 5H), | |
| | | 7.28-7.21 (m, 2H), 7.02-6.96 (m, | |
| | | 2H), 6.79-6.69 (m, 3H), 5.31 (s, | |
| | | 2H), 4.41-4.38 (m, 2H), 4.27 (s, 2H), | |
| | | 3.75 (t, J = 4.4 Hz, 2H), 3.51-3.44 | |
| | | (m, 2H), 3.32 (s, 3H), 2.87-2.83 (m, 2H). | |
| 13 | 656.2 | ¹H NMR (500 MHz, Chloroform-d) δ | 1 |
| | | 8.32 (t, J = 4.9 Hz, 1H), 8.11-8.07 | |
| | | (m, 3H), 7.77-7.64 (m, 2H), 7.48- | |
| | | 7.43 (m, 3H), 7.40-7.34 (m, 2H), | |
| | | 7.28-7.21 (m, 2H), 7.02-6.96 (m, | |
| | | 5H), 6.11 (s, 1H), 5.31 (s, 2H), 4.41- | |
| | | 4.38 (m, 2H), 4.27 (s, 2H), 3.75 (t, J = | |
| | | 4.4 Hz, 2H), 3.51-3.44 (m, 2H), | |
| | | 3.34 (s, 3H), 2.87-2.83 (m, 2H). | |
| 14 | 704.2 | ¹H NMR (500 MHz, Chloroform-d) δ | 2 |
| | | 8.12 (s, 1H), 7.95 (d, J = 8.4 Hz, 1H), | |
| | | 7.79-7.73 (m, 1H), 7.47-7.37 (m, | |
| | | 2H), 7.22-7.16 (m, 3H), 6.82-6.73 | |
| | | (m, 1H), 6.02-6.01 (m, 1H), 5.96 (d, | |
| | | J = 6.8 Hz, 1H), 5.28 (d, J = 1.2 Hz, | |
| | | 2H), 4.38-4.36 (m, 2H), 4.13-4.03 | |
| | | (m, 1H), 3.80-3.71 (m, 4H), 3.36 (s, | |
| | | 3H), 3.07-2.91 (m, 2H), 2.85-2.78 | |
| | | (m, 5H), 2.69-2.60 (m, 2H), 2.12- | |
| | | 1.94 (m, 5H), 1.80-1.77 (m, 1H). | |
| 15 | 684.2 | ¹H NMR (500 MHz, Chloroform-d) δ | 2 |
| | | 8.19-8.16 (m, 1H), 8.08 (d, J = 2.0 | |
| | | Hz, 1H), 7.82-7.78 (m, 1H), 7.42- | |
| | | 7.34 (m, 3H), 7.22-7.14 (m, 3H), | |
| | | 7.02-6.96 (m, 2H), 6.82-6.70 (m, | |
| | | 3H), 6.14 (s, 1H), 5.35 (s, 2H), 4.39- | |
| | | 4.28 (m, 2H), 4.23-4.15 (m, 1H), | |
| | | 3.89-3.73 (m, 2H), 3.71 (s, 3H), | |
| | | 3.55-3.48 (m, 2H), 3.03-3.01 (m, | |
| | | 1H), 2.88-2.74 (m, 4H), 2.65-2.61 | |
| | | (m, 2H), 2.30-2.22 (m, 2H), 2.09- | |
| | | 1.94 (m, 4H). | |
| 16 | 660.1 | ¹H NMR (500 MHz, Chloroform-d) δ | 2 |
| | | 8.10 (t, J = 4.2 Hz, 1H), 8.08 (d, J = | |
| | | 2.0 Hz, 1H), 7.77-7.67 (m, 2H), | |
| | | 7.46-7.39 (m, 3H), 7.21-7.13 (m, | |
| | | 4H), 6.92-6.87 (m, 1H), 6.80-6.76 | |
| | | (m, 1H), 5.31-5.30 (m, 2H), 4.37- | |
| | | 4.33 (m, 2H), 4.05 (t, J = 6.4 Hz, | |
| | | 2H), 3.84-3.75 (m, 4H), 3.40-3.34 | |
| | | (m, 5H), 3.03-3.01 (m, 1H), 2.85- | |
| | | 2.75 (m, 2H), 2.66-2.61 (m, 2H), | |
| | | 2.09-1.94 (m, 6H). | |
| 17 | 711.2 | ¹H NMR (500 MHz, Chloroform-d) δ | 2 |
| | | 9.17 (d, J = 6.8 Hz, 1H), 8.44 (s, | |
| | | 1H), 8.10-8.01 (m, 2H), 7.82- | |
| | | 7.78 (m, 1H), 7.46-7.39 (m, 3H), | |
| | | 7.21-7.13 (m, 4H), 7.08-7.02 (m, | |
| | | 2H), 6.82-6.75 (m, 2H), 5.29 (d, J = | |
| | | 1.2 Hz, 2H), 4.32-4.30 (m, 2H), 3.82- | |
| | | 3.75 (m, 4H), 3.67-3.60 (m, 2H), | |
| | | 3.43-3.35 (m, 1H), 3.31 (s, 3H), | |
| | | 3.03-3.01 (m, 1H), 2.97-2.91 (m, | |
| | | 1H), 2.84-2.75 (m, 2H), 2.69-2.65 | |
| | | (m, 2H), 2.09-1.94 (m, 4H). | |
| 18 | 672.2 | ¹H NMR (500 MHz, Chloroform-d) δ | 2 |
| | | 8.40 (s, 1H),, 8.12 (t, J = 4.9 Hz, 1H), | |
| | | 8.08 (d, J = 2.0 Hz, 1H), 7.81-7.73 | |
| | | (m, 1H), 7.47-7.37 (m, 2H), 7.25- | |
| | | 7.17 (m, 3H), 7.08-7.01 (m, 2H), | |
| | | 6.88-6.77 (m, 3H), 5.26 (d, J = 1.2 | |
| | | Hz, 2H), 4.38-4.36 (m, 2H), 3.79- | |
| | | 3.71 (m, 4H), 3.59 (m, J = 5.1 Hz, | |
| | | 2H), 3.31 (s, 3H), 3.07-2.95 (m, | |
| | | 3H), 2.84-2.74 (m, 2H), 2.68-2.60 | |
| | | (m, 2H), 2.09-1.94 (m, 4H). | |
| 19 | 672.2 | ¹H NMR (500 MHz, Chloroform-d) δ | 2 |
| | | 8.53 (s, 1H), 8.08 (t, J = 4.9 Hz, 1H), | |
| | | 8.08 (d, J = 2.0 Hz, 1H), 7.81-7.73 | |
| | | (m, 1H), 7.46-7.39 (m, 2H), 7.24- | |
| | | 7.15 (m, 3H), 7.08-7.00 (m, 2H), | |
| | | 6.88-6.74 (m, 3H), 5.25 (d, J = 1.2 | |
| | | Hz, 2H), 4.36 (t, J = 4.4 Hz, 2H), | |
| | | 3.79-3.73 (m, 4H), 3.57 (m, J = 5.1 | |
| | | Hz, 2H), 3.32 (s, 3H), 3.07-2.95 (m, | |
| | | 3H), 2.83-2.75 (m, 2H), 2.69-2.65 | |
| | | (m, 2H), 2.09-1.94 (m, 4H). | |
| 20 | 700.2 | ¹H NMR (500 MHz, Chloroform-d) δ | 2 |
| | | 8.23 (t, J = 4.9 Hz, 1H), 8.08 (d, J = | |
| | | 2.0 Hz, 1H), 7.83-7.71 (m, 1H), | |
| | | 7.42-7.33 (m, 2H), 7.21-7.15 (m, | |
| | | 3H), 7.05-6.99 (m, 2H), 6.87-6.82 | |
| | | (m, 2H), 6.81-6.73 (m, 1H), 5.27 (d, | |
| | | J = 1.2 Hz, 2H), 4.33 (t, J = 4.4 Hz, | |
| | | 2H), 3.80-3.75 (m, 6H), 3.44-3.37 | |
| | | (m, 2H), 3.30 (s, 3H), 3.03-3.01 (m, | |
| | | 1H), 2.85-2.75 (m, 2H), 2.68-2.60 | |
| | | (m, 4H), 2.09-1.86 (m, 6H). | |
| 21 | 681.2 | ¹H NMR (500 MHz, Chloroform-d) δ | 2 |
| | | 8.96-8.90 (m, 2H), 8.08 (d, J = 2.0 | |
| | | Hz, 1H), 7.80-7.73 (m, 1H), 7.63 | |
| | | (t, J = 2.2 Hz, 1H), 7.47-7.37 (m, | |
| | | 2H), 7.33 (d, J = 7.3 Hz, 1H), 7.29- | |
| | | 7.23 (m, 1H), 7.22-7.13 (m, 4H), | |
| | | 6.83-6.75 (m, 1H), 6.45-6.40 (m, | |
| | | 1H), 5.29-5.28 (m, 2H), 4.53 (d, J = | |
| | | 6.3 Hz, 2H), 4.37-4.33 (m, 2H), 3.78- | |
| | | 3.73 (m, 4H), 3.31 (s, 3H), 3.03- | |
| | | 3.01 (m, 1H), 2.85-2.76 (m, 2H), | |
| | | 2.68-2.61 (m, 2H), 2.09-1.94 (m, | |
| | | 4H). | |
| 22 | 696.2 | ¹H NMR (500 MHz, Chloroform-d) δ | 2 |
| | | 8.49 (s, 1H), 8.09-8.08 (m, 1H), 8.08 | |
| | | (d, J = 2.0 Hz, 1H), 7.85-7.78 (m, | |
| | | 1H), 7.48-7.39 (m, 2H), 7.21- | |
| | | 7.13 (m, 4H), 7.08-7.02 (m, 2H), | |
| | | 6.82-6.75 (m, 2H), 5.28 (d, J = 1.2 | |
| | | Hz, 2H), 4.32-4.30 (m, 2H), 3.78- | |
| | | 3.70 (m, 4H), 3.67-3.60 (m, 2H), 3.43- | |
| | | 3.35 (m, 4H), 3.07-2.99 (m, 2H), | |
| | | 2.85-2.77 (m, 2H), 2.69-2.61 (m, | |
| | | 2H), 2.09-1.94 (m, 4H). | |
| 23 | 724.2 | ¹H NMR (500 MHz, Chloroform-d) δ | 2 |
| | | 8.14-8.12 (m, 1H), 8.08 (d, J = 2.0 | |
| | | Hz, 1H), 7.82-7.73 (m, 1H), 7.48- | |
| | | 7.35 (m, 2H), 7.21-7.13 (m, 3H), | |
| | | 7.05-6.98 (m, 2H), 6.88-6.72 (m, | |
| | | 3H), , 5.29 (d, J = 1.2 Hz, 2H), 4.34 | |
| | | (t, J = 4.4 Hz, 2H), 3.79-3.70 (m, | |
| | | 4H), 3.65-3.62 (m, 2H), 3.34 (s, 3H), | |
| | | 3.07-2.97 (m, 3H), 2.86-2.77 (m, | |
| | | 2H), 2.69-2.59 (m, 2H), 2.09-1.94 | |
| | | (m, 4H). | |
| 24 | 670.1 | ¹H NMR (500 MHz, Chloroform-d) δ | 2 |
| | | 8.15-8.12 (m, 1H), 8.08 (d, J = 2.0 | |
| | | Hz, 1H), 7.80-7.72 (m, 1H), 7.42- | |
| | | 7.36 (m, 3H), 7.22-7.14 (m, 3H), | |
| | | 7.02-6.96 (m, 2H), 6.81-6.70 (m, | |
| | | 1H), 6.75-6.70 (m, 2H), 6.14 (s, | |
| | | 1H), 5.35 (s, 2H), 4.39-4.30 (m, | |
| | | 2H), 4.23-4.15 (m, 1H), 3.89-3.84 | |
| | | (m, 1H), 3.80-3.73 (m, 2H), 3.71 (s, | |
| | | 2H), 3.03-3.01 (m, 1H), 2.88-2.79 | |
| | | (m, 4H), 2.68-2.59 (m, 2H), 2.30- | |
| | | 2.22 (m, 2H), 2.09-1.94 (m, 4H). | |
| 25 | 686.2 | ¹H NMR (500 MHz, Chloroform-d) δ | 2 |
| | | 8.23 (t, J = 4.9 Hz, 1H), 8.08 (d, J = | |
| | | 2.0 Hz, 1H), 7.81-7.73 (m, 1H), | |
| | | 7.45-7.35 (m, 2H), 7.22-7.15 (m, | |
| | | 3H), 7.05-6.99 (m, 2H), 6.85- | |
| | | 6.76 (m, 3H), 5.31 (d, J = 1.2 Hz, | |
| | | 2H), 4.33 (t, J = 4.4 Hz, 2H), 3.80- | |
| | | 3.75 (m, 3H), 3.44-3.37 (m, 2H), | |
| | | 3.32 (s, 3H), 3.03-3.01 (m, 1H), | |
| | | 2.84-2.74 (m, 2H), 2.68-2.60 (m, | |
| | | 4H), 2.09-1.86 (m, 6H). | |
| 26 | 659.1 | ¹H NMR (500 MHz, Chloroform-d) δ | 2 |
| | | 7.84-7.80 (m, 1H), 7.49 (t, J = 7.7 | |
| | | Hz, 1H), 7.42-7.34 (m, 3H), 7.25- | |
| | | 7.17 (m, 3H), 6.86-6.77 (m, 1H), | |
| | | 5.68 (s, 2H), 5.31 (d, J = 1.2 Hz, 2H), | |
| | | 4.70 (d, J = 2.0 Hz, 1H), 4.64 (d, J = | |
| | | 12.1 Hz, 1H), 4.48 (s, 2H), 4.37-4.33 | |
| | | (m, 2H), 3.79-3.71 (m, 4H), 3.36 (s, | |
| | | 3H), 3.03-3.01 (m, 1H), 2.85-2.74 | |
| | | (m, 2H), 2.69-2.62 (m, 2H), 2.09- | |
| | | 1.94 (m, 4H). | |
| 27 | 699.2 | ¹H NMR (500 MHz, Chloroform-d) δ | 2 |
| | | 9.25 (s, 1H), 8.86 (t, J = 6.3 Hz, 1H), | |
| | | 8.08 (d, J = 2.0 Hz, 1H), 7.79- | |
| | | 7.72 (m, 1H), 7.57-7.51 (m, 2H), | |
| | | 7.46-7.39 (m, 2H), 7.34-7.28 (m, | |
| | | 2H), 7.25-7.12 (m, 3H), 6.88-6.73 | |
| | | (m, 1H), 5.35 (s, 2H), 4.54-4.49 (m, | |
| | | 2H), 4.36-4.31 (m, 2H), 3.81-3.73 | |
| | | (m, 4H), 3.35 (s, 3H), 3.03-3.01 (m, | |
| | | 1H), 2.85-2.75 (m, 2H), 2.65-2.60 | |
| | | (m, 2H), 2.16 (s, 3H), 2.09-1.94 (m, | |
| | | 4H). | |
| 28 | 688.2 | ¹H NMR (500 MHz, Chloroform-d) δ | 2 |
| | | 9.05 (s, 1H), 8.86 (t, J = 6.3 Hz, 1H), | |
| | | 8.08 (d, J = 2.0 Hz, 1H), 7.79- | |
| | | 7.73 (m, 1H), 7.57-7.51 (m, 2H), | |
| | | 7.47-7.37 (m, 2H), 7.34-7.28 (m, | |
| | | 2H), 7.21-7.12 (m, 3H), 6.86-6.74 | |
| | | (m, 1H), 5.35 (s, 2H), 4.54-4.49 (m, | |
| | | 2H), 4.36-4.31 (m, 2H), 4.16 (s, | |
| | | 3H), 3.80-3.73 (m, 4H), 3.31 (s, | |
| | | 3H), 3.03-3.01 (m, 1H), 2.86-2.77 | |
| | | (m, 2H), 2.69-2.65 (m, 2H), 2.09- | |
| | | 1.94 (m, 4H). | |
| 29 | 799.2 | ¹H NMR (500 MHz, Chloroform-d) δ | 1 |
| | | 8.19 (t, J = 4.9 Hz, 1H), 8.04 (d, J = | |
| | | 2.2 Hz, 1H), 7.80 (d, J = 2.1 Hz, 1H), | |
| | | 7.79-7.73 (m, 4H), 7.70-7.66 (m, | |
| | | 1H), 7.50-7.33 (m, 8H), 7.28-7.23 | |
| | | (m, 2H), 7.06 (s, 2H), 6.80-6.75 (m, | |
| | | 1H), 5.31 (s, 2H), 4.41-4.36 (m, | |
| | | 2H), 4.35-4.29 (m, 1H), 4.28-4.22 | |
| | | (m, 1H), 3.88 (s, 2H), 3.75 (t, J = 4.4 | |
| | | Hz, 2H), 3.51-3.44 (m, 2H), 3.34 (s, | |
| | | 3H), 2.92-2.86 (m, 2H). | |
| 30 | 667.2 | ¹H NMR (500 MHz, Chloroform-d) δ | 2 |
| | | 8.86 (t, J = 6.3 Hz, 1H), 8.08 (d, J = | |
| | | 2.0 Hz, 1H), 7.80-7.74 (m, 1H), | |
| | | 7.62-7.59 (m, 2H), 7.50-7.36 (m, | |
| | | 5H), 7.21-7.14 (m, 3H), 6.85-6.74 | |
| | | (m, 1H), 5.35 (s, 2H), 4.56-4.51 (m, | |
| | | 2H), 4.31 (t, J = 4.4 Hz, 2H), 3.80- | |
| | | 3.73 (m, 4H), 3.32 (s, 3H), 3.03- | |
| | | 3.01 (m, 1H), 2.86-2.73 (m, 2H), | |
| | | 2.66-2.61 (m, 2H), 2.09-1.94 (m, | |
| | | 4H). | |
| 31 | 676.1 | ¹H NMR (500 MHz, Chloroform-d) δ | 2 |
| | | 8.82 (t, J = 6.3 Hz, 1H), 8.05 (d, J = | |
| | | 2.0 Hz, 1H), 7.77-7.70 (m, 1H), | |
| | | 7.62-7.59 (m, 2H), 7.50-7.36 (m, | |
| | | 5H), 7.25-7.14 (m, 3H), 6.85-6.78 | |
| | | (m, 1H), 5.35 (s, 2H), 4.56-4.51 (m, | |
| | | 2H), 4.34 (t, J = 4.4 Hz, 2H), 3.80- | |
| | | 3.74 (m, 4H), 3.35 (s, 3H), 3.03- | |
| | | 3.01 (m, 1H), 2.90-2.87 (m, 2H), | |
| | | 2.67-2.61 (m, 2H), 2.09-1.94 (m, | |
| | | 4H). | |
| 32 | 658.1 | ¹H NMR (500 MHz, Chloroform-d) δ | 2 |
| | | 8.79 (t, J = 6.3 Hz, 1H), 8.08 (d, J = | |
| | | 2.0 Hz, 1H), 7.81-7.73 (m, 1H), | |
| | | 7.48-7.37 (m, 2H), 7.21-7.10 (m, | |
| | | 4H), 7.02-6.97 (m, 1H), 6.95 (s, | |
| | | 1H), 6.86-6.81 (m, 2H), , 6.73- | |
| | | 6.67 (m, 1H), 5.35 (s, 2H), 4.47- | |
| | | 4.42 (m, 2H), 4.38-4.36 (m, 2H), 3.81- | |
| | | 3.74 (m, 4H), 3.31 (s, 3H), 3.03- | |
| | | 3.01 (m, 1H), 2.83-2.74 (m, 2H), | |
| | | 2.68-2.61 (m, 2H), 2.09-1.94 (m, | |
| | | 4H). | |
| 33 | 674.1 | ¹H NMR (500 MHz, Chloroform-d) δ | 2 |
| | | 8.72 (t, J = 6.3 Hz, 1H), 8.08 (d, J = | |
| | | 2.0 Hz, 1H), 7.49-7.37 (m, 2H), | |
| | | 7.21-7.10 (m, 4H), 7.02-6.97 (m, | |
| | | 2H), 6.83-6.73 (m, 3H), 5.35 (s, | |
| | | 2H), 4.47-4.42 (m, 2H), 4.38-4.36 | |
| | | (m, 2H), 3.82-3.75 (m, 4H), 3.36 (s, | |
| | | 3H), 3.03-3.01 (m, 1H), 2.83-2.75 | |
| | | (m, 2H), 2.68-2.59 (m, 2H), 2.09- | |
| | | 1.94 (m, 4H). | |
| 34 | 659.1 | ¹H NMR (500 MHz, Chloroform-d) δ | 2 |
| | | 8.91 (t, J = 6.3 Hz, 1H), 8.05 (d, J = | |
| | | 2.0 Hz, 1H), 7.77-7.70 (m, 1H), | |
| | | 7.62-7.59 (m, 2H), 7.50-7.36 (m, | |
| | | 5H), 7.25-7.18 (m, 3H), 6.84-6.78 | |
| | | (m, 1H), 5.35 (s, 2H), 4.56-4.51 (m, | |
| | | 2H), 4.33 (t, J = 4.4 Hz, 2H), 3.83- | |
| | | 3.75 (m, 4H), 3.37 (s, 3H), 3.03- | |
| | | 3.01 (m, 1H), 2.84-2.75 (m, 2H), | |
| | | 2.67-2.59 (m, 2H), 2.09-1.94 (m, | |
| | | 4H). | |
| 35 | 658.1 | ¹H NMR (500 MHz, Chloroform-d) δ | 2 |
| | | 8.79 (t, J = 6.3 Hz, 1H), 8.08 (d, J = | |
| | | 2.0 Hz, 1H), 7.82-7.74 (m, 1H), | |
| | | 7.47-7.37 (m, 2H), 7.21-7.10 (m, | |
| | | 4H), 7.02-6.97 (m, 1H), 6.86-6.79 | |
| | | (m, 2H), 6.73-6.63 (m, 2H), 5.35 (s, | |
| | | 2H), 4.47-4.42 (m, 2H), 4.37-4.33 | |
| | | (m, 2H), 3.81-3.76 (m, 4H), 3.30 (s, | |
| | | 3H), 3.03-3.01 (m, 1H), 2.84-2.74 | |
| | | (m, 2H), 2.67-2.58 (m, 2H), 2.09- | |
| | | 1.94 (m, 4H). | |
| 36 | 716.2 | ¹H NMR (500 MHz, Chloroform-d) δ | 2 |
| | | 8.88 (t, J = 5.8 Hz, 1H), 8.08 (d, J = | |
| | | 2.0 Hz, 1H), 7.82-7.80 (m, 1H), | |
| | | 7.83-7.74 (m, 1H), 7.46 (d, J = 15.4 | |
| | | Hz, 1H), 7.42-7.38 (m, 2H), 7.27- | |
| | | 7.13 (m, 4H), 6.90 (d, J = 8.0 Hz, | |
| | | 1H), 6.84-6.75 (m, 1H), 5.35 (s, | |
| | | 2H), 4.57-4.52 (m, 2H), 4.34 (t, J = | |
| | | 4.4 Hz, 2H), 3.86 (s, 3H), 3.81-3.73 | |
| | | (m, 4H), 3.32 (s, 3H), 3.03-3.01 (m, | |
| | | 1H), 2.83-2.74 (m, 2H), 2.67-2.58 | |
| | | (m, 2H), 2.09-1.94 (m, 4H). | |
| 37 | 702.1 | ¹H NMR (500 MHz, Chloroform-d) δ | 2 |
| | | 8.89 (t, J = 5.8 Hz, 1H), 8.08 (d, J = | |
| | | 2.0 Hz, 1H), 7.82-7.80 (m, 1H), | |
| | | 7.78-7.72 (m, 1H), 7.42-7.38 (m, | |
| | | 3H), 7.27-7.13 (m, 4H), 6.90 (d, J = | |
| | | 8.0 Hz, 1H), 6.81-6.75 (m, 1H), | |
| | | 5.35 (s, 2H), 4.57-4.52 (m, 2H), | |
| | | 4.32-4.30 (m, 2H), 3.81-3.74 (m, | |
| | | 4H), 3.31 (s, 3H), 3.03-3.01 (m, | |
| | | 1H), 2.83-2.75 (m, 2H), 2.67-2.59 | |
| | | (m, 2H), 2.09-1.94 (m, 4H). | |
| 38 | 698.2 | ¹H NMR (500 MHz, Chloroform-d) δ | 2 |
| | | 9.75 (s, 1H), 9.49 (s, 1H), 8.80 (t, J = | |
| | | 6.3 Hz, 1H), 8.04-7.99 (m, 2H), | |
| | | 7.82-7.72 (m, 1H), 7.46-7.37 (m, | |
| | | 2H), 7.21-7.16 (m, 1H), 7.18-7.13 | |
| | | (m, 2H), 7.13-7.05 (m, 2H), 6.81- | |
| | | 6.73 (m, 1H), 5.35 (s, 2H), 4.50 (d, J = | |
| | | 6.3 Hz, 2H), 4.38-4.36 (m, 2H), | |
| | | 3.81-3.76 (m, 4H), 3.35 (s, 3H), | |
| | | 3.03-3.01 (m, 1H), 2.85-2.75 (m, | |
| | | 2H), 2.65-2.59 (m, 2H), 2.09-1.94 (m, 4H). | |
| 39 | 802.3 | ¹H NMR (500 MHz, Chloroform-d) δ | 2 |
| | | 9.20 (d, J = 7.1 Hz, 1H), 8.03 (t, J = | |
| | | 4.9 Hz, 1H), 8.08 (d, J = 2.0 Hz, 1H), | |
| | | 7.84-7.76 (m, 1H), 7.55 (d, J = 2.4 | |
| | | Hz, 1H), 7.45-7.37 (m, 2H), 7.32 | |
| | | (d, J = 7.1 Hz, 1H), 7.21-7.16 (m, | |
| | | 4H), 7.09 (d, J = 7.1 Hz, 1H), 6.89- | |
| | | 6.79 (m, 1H), 5.86-5.79 (m, 1H), | |
| | | 5.35 (s, 2H), 4.36-4.31 (m, 4H), | |
| | | 3.82-3.76 (m, 4H), 3.63-3.57 (m, | |
| | | 2H), 3.43-3.35 (m, 1H), 3.37 (s, | |
| | | 3H), 3.07-3.00 (m, 2H), 2.84-2.75 | |
| | | (m, 2H), 2.65-2.58 (m, 2H), 2.57 (d, | |
| | | J = 6.3 Hz, 3H), 2.09-1.94 (m, 4H). | |
| 40 | 672.2 | ¹H NMR (500 MHz, Chloroform-d) δ | 2 |
| | | 8.39-8.37 (m, 1H), 8.08 (d, J = 2.1 | |
| | | Hz, 1H), 7.84-7.78 (m, 1H), 7.45- | |
| | | 7.37 (m, 2H), 7.31 (s, 1H), 7.24- | |
| | | 7.13 (m, 5H), 6.89-6.81 (m, 1H), | |
| | | 6.74-6.68 (m, 2H), 5.35 (s, 2H), | |
| | | 5.17-5.07 (m, 1H), 4.36 (t, J = 4.4 | |
| | | Hz, 2H), 3.83-3.76 (m, 4H), 3.34 (s, | |
| | | 3H), 3.03-3.01 (m, 1H), 2.84-2.76 | |
| | | (m, 2H), 2.66-2.58 (m, 2H), 2.09- | |
| | | 1.94 (m, 4H), 1.52 (s, 3H). | |
| 41 | 714.2 | ¹H NMR (500 MHz, Chloroform-d) δ | 2 |
| | | 8.12 (s, 1H), 7.95-7.88 (m, 1H), | |
| | | 7.83-7.75 (m, 1H), 7.46-7.39 (m, | |
| | | 3H), 7.29-7.13 (m, 8H), 6.86-6.78 | |
| | | (m, 1H), 5.35 (s, 2H), 4.33 (t, J = 4.4 | |
| | | Hz, 2H), 3.83-3.75 (m, 4H), 3.59- | |
| | | 3.49 (m, 3H), 3.36 (s, 3H), 3.03- | |
| | | 3.01 (m, 1H), 2.90-2.87 (m, 2H), | |
| | | 2.75-2.65 (m, 2H), 2.68-2.60 (m, | |
| | | 2H), 2.09-1.94 (m, 4H). | |
| 42 | 702.1 | ¹H NMR (500 MHz, Chloroform-d) δ | 2 |
| | | 8.55 (s, 1H), 8.38 (t, J = 5.9 Hz, 1H), | |
| | | 8.08 (d, J = 2.0 Hz, 1H), 7.85- | |
| | | 7.77 (m, 1H), 7.54-7.47 (m, 2H), | |
| | | 7.42-7.34 (m, 3H), 7.22-7.15 (m, | |
| | | 3H), 6.86 (d, J = 9.3 Hz, 1H), 6.86- | |
| | | 6.74 (m, 1H), 5.35 (s, 2H), 4.93 (d, J = | |
| | | 6.0 Hz, 2H), 4.38-4.36 (m, 2H), | |
| | | 3.82-3.75 (m, 4H), 3.35 (s, 3H), | |
| | | 3.03-3.01 (m, 1H), 2.85-2.76 (m, | |
| | | 2H), 2.63-2.58 (m, 2H), 2.09-1.94 | |
| | | (m, 4H). | |
| 43 | 698.2 | ¹H NMR (500 MHz, Chloroform-d) δ | 2 |
| | | 7.99-7.96 (m, 1H), 7.79-7.73 (m, | |
| | | 1H), 7.67 (d, J = 7.5 Hz, 1H), 7.45- | |
| | | 7.35 (m, 2H), 7.22-7.13 (m, 3H), | |
| | | 6.93 (d, J = 1.1 Hz, 1H), 6.84-6.74 | |
| | | (m, 1H), 6.52 (t, J = 1.0 Hz, 1H), | |
| | | 5.31-5.30 (m, 2H), 5.24 (s, 1H), 4.38- | |
| | | 4.29 (m, 3H), 3.82-3.73 (m, 4H), | |
| | | 3.31 (s, 3H), 3.11-3.00 (m, 3H), | |
| | | 3.00-2.93 (m, 2H), 2.87-2.79 (m, | |
| | | 2H), 2.63-2.55 (m, 2H), 2.16 (s, | |
| | | 3H), 2.09-1.94 (m, 4H). | |
| 44 | 728.1 | ¹H NMR (500 MHz, Chloroform-d) δ | 2 |
| | | 8.22-8.19 (m, 1H), 8.08 (d, J = 2.4 | |
| | | Hz, 1H), 7.83-7.77 (m, 1H), 7.50- | |
| | | 7.42 (m, 3H), 7.39-7.32 (m, 1H), | |
| | | 7.15-7.01 (m, 2H), 6.85-6.74 (m, | |
| | | 1H), 5.35 (s, 2H), 4.94-4.89 (m, | |
| | | 1H), 4.49-4.43 (m, 1H), 4.39-4.30 | |
| | | (m, 2H), 4.23-4.15 (m, 1H), 4.06- | |
| | | 3.93 (m, 2H), 3.89-3.84 (m, 1H), | |
| | | 3.79-3.76 (m, 1H), 3.78-3.74 (m, | |
| | | 1H), 3.71 (s, 3H), 3.67-3.55 (m, | |
| | | 1H), 3.03-3.01 (m, 1H), 2.90-2.87 | |
| | | (m, 2H), 2.65-2.55 (m, 2H), 2.53- | |
| | | 2.47 (m, 1H), 2.33-2.18 (m, 3H), | |
| | | 2.09-1.94 (m, 4H). | |
| 45 | 735.2 | ¹H NMR (500 MHz, Chloroform-d) δ | 2 |
| | | 8.22-8.19 (m, 1H), 8.08 (d, J = 2.4 | |
| | | Hz, 1H), 7.81-7.70 (m, 1H), 7.50- | |
| | | 7.42 (m, 3H), 7.37-7.34 (m, 1H), | |
| | | 7.15-7.01 (m, 2H), 6.86-6.72 (m, | |
| | | 1H), 5.35 (s, 2H), 4.94-4.89 (m, | |
| | | 1H), 4.49-4.43 (m, 1H), 4.39-4.30 | |
| | | (m, 2H), 4.23-4.15 (m, 1H), 4.06- | |
| | | 3.93 (m, 2H), 3.89-3.84 (m, 1H), | |
| | | 3.79-3.76 (m, 1H), 3.78-3.74 (m, | |
| | | 1H), 3.71 (s, 3H), 3.67-3.55 (m, | |
| | | 1H), 3.03-3.01 (m, 1H), 2.83-2.74 | |
| | | (m, 2H), 2.65-2.56 (m, 2H), 2.53- | |
| | | 2.47 (m, 1H), 2.33-2.18 (m, 3H), | |
| | | 2.09-1.94 (m, 4H). | |
| 46 | 735.2 | ¹H NMR (500 MHz, Chloroform-d) δ | 2 |
| | | 8.20-8.17 (m, 1H), 8.08 (d, J = 2.4 | |
| | | Hz, 1H), 7.82-7.78 (m, 1H), 7.50- | |
| | | 7.42 (m, 3H), 7.39-7.32 (m, 1H), | |
| | | 7.15-7.01 (m, 2H), 6.88-6.76 (m, | |
| | | 1H), 5.35 (s, 2H), 4.94-4.89 (m, | |
| | | 1H), 4.49-4.43 (m, 1H), 4.39-4.30 | |
| | | (m, 2H), 4.23-4.15 (m, 1H), 4.06- | |
| | | 3.93 (m, 2H), 3.89-3.84 (m, 1H), | |
| | | 3.79-3.76 (m, 1H), 3.78-3.74 (m, | |
| | | 1H), 3.71 (s, 3H), 3.67-3.55 (m, | |
| | | 1H), 3.03-3.01 (m, 1H), 2.85-2.74 | |
| | | (m, 2H), 2.65-2.57 (m, 2H), 2.53- | |
| | | 2.47 (m, 1H), 2.33-2.18 (m, 3H), | |
| | | 2.08-1.94 (m, 4H). | |
| 47 | 740.3 | ¹H NMR (500 MHz, Chloroform-d) δ | 2 |
| | | 8.22-8.19 (m, 1H), 8.08 (d, J = 2.4 | |
| | | Hz, 1H), 7.83-7.80 (m, 1H), 7.50- | |
| | | 7.42 (m, 3H), 7.39-7.32 (m, 1H), | |
| | | 7.15-7.01 (m, 2H), 6.86-6.78 (m, | |
| | | 1H), 5.35 (s, 2H), 4.94-4.89 (m, | |
| | | 1H), 4.49-4.43 (m, 1H), 4.39-4.30 | |
| | | (m, 2H), 4.23-4.15 (m, 1H), 4.06- | |
| | | 3.93 (m, 2H), 3.89-3.84 (m, 1H), | |
| | | 3.79-3.76 (m, 1H), 3.78-3.74 (m, | |
| | | 1H), 3.71 (s, 3H), 3.67-3.55 (m, | |
| | | 1H), 3.25 (s, 3H), 3.03-3.01 (m, | |
| | | 1H), 2.85-2.72 (m, 2H), 2.65-2.58 | |
| | | (m, 2H), 2.53-2.47 (m, 1H), 2.33- | |
| | | 2.18 (m, 3H), 2.09-1.94 (m, 4H). | |
| 48 | 720.3 | ¹H NMR (500 MHz, Chloroform-d) δ | 2 |
| | | 8.14 (t, J = 5.9 Hz, 1H), 8.02 (d, J = | |
| | | 2.4 Hz, 1H), 7.70-7.59 (m, 2H), 7.51- | |
| | | 7.47 (m, 2H), 7.34 (s, 1H), 7.18- | |
| | | 7.15 (m, 1H), 6.83-6.81 (m, 1H), 5.32 | |
| | | (s, 2H), 4.95-4.80 (m, 3H), 4.44- | |
| | | 4.38 (m, 2H), 4.32-4.30 (m, 1H), | |
| | | 4.19-4.16 (m, 1H), 4.01-3.82 (m, | |
| | | 6H), 3.72 (s, 2H), 3.01-2.78 (m, 3H), | |
| | | 2.69-2.61 (m, 2H), 2.42-2.31 (m, | |
| | | 3H), 2.01-1.90 (m, 6H). | |
| 49 | 709.1 | ¹H NMR (500 MHz, Chloroform-d) δ | 2 |
| | | 8.24-8.21 (m, 1H), 8.08 (d, J = 2.0 | |
| | | Hz, 1H), 7.90 (d, J = 9.3 Hz, 1H), | |
| | | 7.81-7.75 (m, 1H), 7.48-7.40 (m, | |
| | | 2H), 7.35 (d, J = 8.8 Hz, 1H), 7.21- | |
| | | 7.12 (m, 4H), 6.86-6.75 (m, 1H), | |
| | | 5.31-5.30 (m, 2H), 4.39-4.30 (m, | |
| | | 2H), 4.24-4.13 (m, 2H), 4.01 (t, J = | |
| | | 5.6 Hz, 1H), 3.92-3.88 (m, 1H), | |
| | | 3.90-3.78 (m, 2H), 3.80-3.75 (m, | |
| | | 1H), 3.71 (s, 2H), 3.70-3.62 (m, | |
| | | 1H), 3.03-3.01 (m, 1H), 2.84-2.74 | |
| | | (m, 2H), 2.64-2.56 (m, 2H), 2.36- | |
| | | 2.31 (m, 1H), 2.09-1.94 (m, 5H). | |
| 50 | 622.1 | ¹H NMR (500 MHz, Chloroform-d) δ | 2 |
| | | 8.24 (t, J = 6.7 Hz, 1H), 8.08 (d, J = | |
| | | 2.0 Hz, 1H), 7.82-7.74 (m, 1H), | |
| | | 7.46-7.32 (m, 3H), 7.21-7.13 (m, | |
| | | 3H), 6.86-6.78 (m, 1H), 5.35 (s, | |
| | | 2H), 4.39-4.30 (m, 2H), 4.23- | |
| | | 4.15 (m, 1H), 3.90 (s, 1H), 3.87-3.85 | |
| | | (m, 1H), 3.78-3.74 (m, 1H), 3.71 (s, | |
| | | 2H), 3.03-3.01 (m, 1H), 2.83-2.74 | |
| | | (m, 2H), 2.64-2.54 (m, 2H), 2.36- | |
| | | 2.31 (m, 2H), 2.09-1.94 (m, 4H). | |
| 51 | 728.2 | ¹H NMR (500 MHz, Chloroform-d) δ | 2 |
| | | 8.49-8.46 (m, 1H), 7.99-7.96 (m, | |
| | | 1H), 7.81-7.77 (m, 1H), 7.42-7.33 | |
| | | (m, 3H), 7.21-7.12 (m, 3H), 6.98- | |
| | | 6.92 (m, 2H), 6.82-6.71 (m, 3H), | |
| | | 6.14 (s, 1H), 5.31-5.30 (m, 2H), 4.59- | |
| | | 4.50 (m, 1H), 4.39-4.30 (m, 2H), | |
| | | 4.23-4.15 (m, 1H), 3.91-3.83 (m, | |
| | | 1H), 3.78-3.74 (m, 1H), 3.71 (s, | |
| | | 2H), 3.08-3.03 (m, 1H), 3.06-2.99 | |
| | | (m, 2H), 2.83-2.73 (m, 2H), 2.65- | |
| | | 2.54 (m, 2H), 2.36-2.31 (m, 1H), | |
| | | 2.09-1.94 (m, 5H). | |
| 52 | 679.1 | ¹H NMR (500 MHz, Chloroform-d) δ | 2 |
| | | 8.71 (d, J = 9.2 Hz, 1H), 7.99-7.96 | |
| | | (m, 1H), 7.83-7.80 (m, 1H), 7.42- | |
| | | 7.33 (m, 3H), 7.21-7.12 (m, 4H), | |
| | | 6.85-6.78 (m, 1H), 5.35 (s, 2H), | |
| | | 4.69-4.60 (m, 1H), 4.39-4.30 (m, | |
| | | 2H), 4.23-4.15 (m, 1H), 3.91-3.83 | |
| | | (m, 1H), 3.78-3.74 (m, 1H), 3.71 (s, | |
| | | 2H), 3.03-3.01 (m, 1H), 2.83-2.73 | |
| | | (m, 3H), 2.73-2.63 (m, 2H), 2.69- | |
| | | 2.65 (m, 1H), 2.36-2.31 (m, 1H), | |
| | | 2.09-1.94 (m, 5H). | |
| 53 | 702.1 | ¹H NMR (500 MHz, Chloroform-d) δ | 2 |
| | | 8.82 (d, J = 8.8 Hz, 1H), 7.99-7.96 | |
| | | (m, 1H), 7.82-7.77 (m, 1H), 7.42- | |
| | | 7.38 (m, 2H), 7.40-7.33 (m, 2H), | |
| | | 7.21-7.11 (m, 3H), 6.86-6.78 (m, | |
| | | 1H), 6.58 (d, J = 1.8 Hz, 1H), 5.27 (d, | |
| | | J = 1.2 Hz, 2H), 4.72-4.63 (m, 1H), | |
| | | 4.39-4.30 (m, 2H), 4.23-4.15 (m, | |
| | | 1H), 3.91-3.83 (m, 1H), 3.78-3.74 | |
| | | (m, 1H), 3.71 (s, 2H), 3.12-2.95 (m, | |
| | | 3H), 2.84-2.73 (m, 2H), 2.65-2.55 | |
| | | (m, 2H), 2.36-2.31 (m, 1H), 2.11- | |
| | | 1.95 (m, 5H). | |
| 54 | 719.2 | ¹H NMR (500 MHz, Chloroform-d) δ | 2 |
| | | 8.23 (t, J = 5.6 Hz, 1H), 8.08 (d, J = | |
| | | 2.4 Hz, 1H), 7.83-7.76 (m, 1H), | |
| | | 7.42-7.32 (m, 3H), 7.22-7.11 (m, | |
| | | 3H), 6.86-6.76 (m, 1H), 5.31-5.30 | |
| | | (m, 2H), 4.39-4.30 (m, 2H), 4.23- | |
| | | 4.15 (m, 1H), 4.06-3.93 (m, 2H), | |
| | | 3.89-3.84 (m, 1H), 3.80-3.73 (m, | |
| | | 1H), 3.71-3.61 (m, 3H), 3.63-3.54 | |
| | | (m, 2H), 3.49-3.40 (m, 1H), 3.03- | |
| | | 3.01 (m, 1H), 2.86-2.82 (m, 1H), | |
| | | 2.84-2.74 (m, 2H), 2.63-2.55 (m, | |
| | | 2H), 2.30-2.22 (m, 2H), 2.15-1.85 | |
| | | (m, 6H). | |
| 55 | 759.2 | ¹H NMR (500 MHz, Chloroform-d) δ | 2 |
| | | 8.35-8.31 (m, 1H), 8.08 (d, J = 2.1 | |
| | | Hz, 1H), 7.81-7.74 (m, 1H), 7.42- | |
| | | 7.32 (m, 3H), 7.22-7.12 (m, 3H), | |
| | | 6.80-6.77 (m, 1H), 5.26 (d, J = 1.2 | |
| | | Hz, 2H), 4.51-4.46 (m, 1H), 4.39- | |
| | | 4.30 (m, 2H), 4.23-4.15 (m, 1H), | |
| | | 4.06-3.93 (m, 2H), 3.89-3.84 (m, | |
| | | 1H), 3.80-3.73 (m, 1H), 3.71 (s, | |
| | | 2H), 3.03-3.01 (m, 1H), 2.85-2.74 | |
| | | (m, 2H), 2.63-2.55 (m, 2H), 2.30- | |
| | | 2.22 (m, 2H), 2.16-2.11 (m, 1H), | |
| | | 2.09-1.94 (m, 4H), 1.90-1.79 (m, | |
| | | 2H), 1.51-1.48 (m, 1H), 1.35 (s, | |
| | | 3H), 1.01 (s, 3H). | |
| 56 | 725.7 | ¹H NMR (500 MHz, Chloroform-d) δ | 2 |
| | | 8.23 (t, J = 5.6 Hz, 1H), 8.08 (d, J = | |
| | | 2.4 Hz, 1H), 7.82-7.73 (m, 1H), | |
| | | 7.42-7.32 (m, 3H), 7.25-7.14 (m, | |
| | | 3H), 6.84-6.78 (m, 1H), 5.31-5.30 | |
| | | (m, 2H), 4.39-4.30 (m, 2H), 4.23- | |
| | | 4.15 (m, 1H), 4.06-3.93 (m, 2H), | |
| | | 3.89-3.84 (m, 1H), 3.80-3.73 (m, | |
| | | 1H), 3.71-3.61 (m, 3H), 3.63-3.54 | |
| | | (m, 2H), 3.49-3.40 (m, 1H), 3.03- | |
| | | 3.01 (m, 1H), 2.86-2.82 (m, 1H), | |
| | | 2.85-2.73 (m, 2H), 2.66-2.55 (m, | |
| | | 2H), 2.30-2.22 (m, 2H), 2.15-1.85 | |
| | | (m, 6H). | |
| 57 | 749.2 | ¹H NMR (500 MHz, Chloroform-d) δ | 2 |
| | | 8.04-8.02 (m, 1H), 7.95 (d, J = 8.4 | |
| | | Hz, 1H), 7.80-7.73 (m, 1H), 7.42- | |
| | | 7.32 (m, 3H), 7.22-7.18 (m, 3H), | |
| | | 6.83-6.77 (m, 1H), 5.31-5.30 (m, | |
| | | 2H), 4.63-4.58 (m, 1H), 4.51-4.40 | |
| | | (m, 2H), 4.39-4.30 (m, 2H), 4.23- | |
| | | 4.15 (m, 1H), 3.89-3.84 (m, 1H), | |
| | | 3.80-3.73 (m, 1H), 3.71 (s, 2H), | |
| | | 3.51-3.48 (m, 2H), 3.35 (d, J = 5.5 | |
| | | Hz, 1H), 3.03-3.01 (m, 1H), 2.83- | |
| | | 2.73 (m, 2H), 2.66-2.58 (m, 2H), | |
| | | 2.49-2.46 (m, 1H), 2.30-2.13 (m, | |
| | | 3H), 2.09-1.94 (m, 4H), 1.37 (d, J = | |
| | | 6.2 Hz, 3H). | |
| 58 | 747.2 | ¹H NMR (500 MHz, Chloroform-d) δ | 2 |
| | | 8.22-8.19 (m, 1H), 8.08 (d, J = 2.1 | |
| | | Hz, 1H), 7.80-7.77 (m, 1H), 7.42- | |
| | | 7.33 (m, 3H), 7.22-7.15 (m, 3H), | |
| | | 6.80-6.77 (m, 1H), 5.28 (d, J = 1.2 | |
| | | Hz, 2H), 4.43-4.39 (m, 1H), 4.39- | |
| | | 4.30 (m, 2H), 4.23-4.15 (m, 1H), | |
| | | 4.01-3.91 (m, 2H), 3.88-3.81 (m, | |
| | | 1H), 3.80-3.73 (m, 1H), 3.71 (s, | |
| | | 2H), 3.55-3.51 (m, 1H), 3.39 (d, J = | |
| | | 12.4 Hz, 1H), 3.03-3.01 (m, 1H), | |
| | | 2.83-2.74 (m, 2H), 2.66-2.59 (m, | |
| | | 2H), 2.30-2.22 (m, 2H), 2.15-2.09 | |
| | | (m, 1H), 2.09-1.94 (m, 4H), 1.87- | |
| | | 1.81 (m, 1H), 1.04 (s, 3H), 0.99 (s, | |
| | | 3H). | |
| 59 | 745.2 | ¹H NMR (500 MHz, Chloroform-d) δ | 2 |
| | | 8.42 (t, J = 6.1 Hz, 1H), 8.08 (d, J = | |
| | | 2.4 Hz, 1H), 7.80-7.74 (m, 1H), | |
| | | 7.42-7.33 (m, 3H), 7.22-7.13 (m, | |
| | | 3H), 6.84-6.74 (m, 1H), 5.35 (s, | |
| | | 2H), 4.39-4.32 (m, 2H), 4.32-4.23 | |
| | | (m, 2H), 4.23-4.15 (m, 1H), 4.05- | |
| | | 3.97 (m, 1H), 3.96-3.92 (m, 1H), | |
| | | 3.89-3.84 (m, 1H), 3.80-3.73 (m, | |
| | | 1H), 3.71 (s, 2H), 3.03-3.01 (m, | |
| | | 1H), 2.83-2.75 (m, 2H), 2.66-2.57 | |
| | | (m, 2H), 2.46-2.38 (m, 1H), 2.30- | |
| | | 2.22 (m, 2H), 2.09-1.86 (m, 7H), | |
| | | 1.85-1.71 (m, 3H). | |
| 60 | 719.2 | ¹H NMR (500 MHz, Chloroform-d) δ | 2 |
| | | 8.23 (t, J = 5.6 Hz, 1H), 8.08 (d, J = | |
| | | 2.4 Hz, 1H), 7.81-7.74 (m, 1H), | |
| | | 7.42-7.35 (m, 3H), 7.25-7.13 (m, | |
| | | 3H), 6.83-6.74 (m, 1H), 5.31-5.30 | |
| | | (m, 2H), 4.39-4.30 (m, 2H), 4.23- | |
| | | 4.15 (m, 1H), 4.06-3.93 (m, 2H), | |
| | | 3.89-3.84 (m, 1H), 3.80-373 (m, | |
| | | 1H), 3.71-3.61 (m, 3H), 3.63-3.54 | |
| | | (m, 2H), 3.49-3.40 (m, 1H), 3.03- | |
| | | 3.01 (m, 1H), 2.86-2.82 (m, 1H), | |
| | | 2.83-2.73 (m, 2H), 2.65-2.57 (m, | |
| | | 2H), 2.35-2.23 (m, 2H), 2.15-1.95 | |
| | | (m, 6H). | |
| 61 | 705.1 | ¹H NMR (500 MHz, Chloroform-d) δ | 2 |
| | | 8.22-8.19 (m, 1H), 8.08 (d, J = 2.1 | |
| | | Hz, 1H), 7.83-7.75 (m, 1H), 7.42- | |
| | | 7.38 (m, 3H), 7.21-7.12 (m, 3H), | |
| | | 6.83-6.76 (m, 1H), 5.31-5.30 (m, | |
| | | 2H), 4.51-4.45 (m, 1H), 4.39-4.30 | |
| | | (m, 2H), 4.23-4.15 (m, 1H), 4.03- | |
| | | 3.89 (m, 2H), 3.89-3.84 (m, 1H), | |
| | | 3.80-3.66 (m, 2H), 3.71 (s, 2H), | |
| | | 3.63-3.53 (m, 1H), 3.03-3.01 (m, | |
| | | 1H), 2.84-2.73 (m, 2H), 2.65-2.57 | |
| | | (m, 2H), 2.38-2.28 (m, 1H), 2.31- | |
| | | 2.23 (m, 1H), 2.26-2.19 (m, 1H), | |
| | | 2.20-2.14 (m, 1H), 2.09-1.94 (m, | |
| | | 4H). | |
| 62 | 755.1 | ¹H NMR (500 MHz, Chloroform-d) δ | 2 |
| | | 8.22-8.19 (m, 1H), 8.08 (d, J = 2.4 | |
| | | Hz, 1H), 7.81-7.75 (m, 1H), 7.49 (t, | |
| | | J = 7.7 Hz, 1H), 7.42-7.38 (m, 1H), | |
| | | 7.36-7.32 (m, 1H), 7.21-7.12 (m, | |
| | | 3H), 6.81-6.75 (m, 1H), 5.31-5.30 | |
| | | (m, 2H), 4.62-4.56 (m, 1H), 4.39- | |
| | | 4.30 (m, 2H), 4.23-4.15 (m, 1H), | |
| | | 4.04-3.96 (m, 1H), 3.98-3.83 (m, | |
| | | 3H), 3.86-3.81 (m, 1H), 3.83-3.73 | |
| | | (m, 1H), 3.71 (s, 2H), 3.03-3.01 (m, | |
| | | 1H), 2.84-2.75 (m, 2H), 2.74-2.52 | |
| | | (m, 4H), 2.30-2.22 (m, 2H), 2.09- | |
| | | 1.94 (m, 4H). | |
| 63 | 749.2 | ¹H NMR (500 MHz, Chloroform-d) δ | 2 |
| | | 8.22-8.19 (m, 1H), 8.08 (d, J = 2.1 | |
| | | Hz, 1H), 7.83-7.80 (m, 1H), 7.45- | |
| | | 7.41 (m, 3H), 7.25-7.17 (m, 3H), | |
| | | 6.81-6.74 (m, 1H), 5.31-5.30 (m, | |
| | | 2H), 4.49-4.43 (m, 1H), 4.39-4.32 | |
| | | (m, 1H), 4.32-4.24 (m, 2H), 4.23- | |
| | | 4.15 (m, 1H), 4.06-3.93 (m, 2H), | |
| | | 3.89-3.84 (m, 1H), 3.80-3.73 (m, | |
| | | 1H), 3.73-3.58 (m, 8H), 3.03-3.01 | |
| | | (m, 1H), 2.84-2.77 (m, 2H), 2.65- | |
| | | 2.58 (m, 2H), 2.36-2.17 (m, 3H), | |
| | | 2.09-1.94 (m, 5H). | |
| 64 | 769.2 | ¹H NMR (500 MHz, Chloroform-d) δ | 2 |
| | | 8.22-8.19 (m, 1H), 8.08 (d, J = 2.1 | |
| | | Hz, 1H), 7.82-7.78 (m, 1H), 7.47- | |
| | | 7.36 (m, 3H), 7.25-7.18 (m, 3H), | |
| | | 6.80-6.73 (m, 1H), 5.31-5.30 (m, | |
| | | 2H), 4.49-4.43 (m, 1H), 4.39-4.30 | |
| | | (m, 2H), 4.23-4.15 (m, 1H), 4.06- | |
| | | 3.93 (m, 2H), 3.89-3.84 (m, 1H), | |
| | | 3.80-3.73 (m, 1H), 3.73-3.58 (m, | |
| | | 7H), 3.03-3.01 (m, 1H), 2.85-2.77 | |
| | | (m, 2H), 2.65-2.59 (m, 2H), 2.36- | |
| | | 2.17 (m, 3H), 2.09-1.94 (m, 5H). | |
| 65 | 749.2 | ¹H NMR (500 MHz, Chloroform-d) δ | 2 |
| | | 8.04-8.02 (m, 1H), 7.95 (d, J = 8.4 | |
| | | Hz, 1H), 7.83-7.80 (m, 1H), 7.47- | |
| | | 7.34 (m, 3H), 7.25-7.18 (m, 3H), | |
| | | 6.83-6.75 (m, 1H), 5.31-5.30 (m, | |
| | | 2H), 4.63-4.58 (m, 1H), 4.51-4.40 | |
| | | (m, 2H), 4.39-4.30 (m, 2H), 4.23- | |
| | | 4.15 (m, 1H), 3.89-3.84 (m, 1H), | |
| | | 3.80-3.73 (m, 1H), 3.71 (s, 2H), | |
| | | 3.51-3.48 (m, 2H), 3.35 (d, J = 5.5 | |
| | | Hz, 1H), 3.03-3.01 (m, 1H), 2.85- | |
| | | 2.74 (m, 2H), 2.65-2.55 (m, 2H), | |
| | | 2.49-2.46 (m, 1H), 2.30-2.13 (m, | |
| | | 3H), 2.09-1.94 (m, 4H), 1.37 (d, J = | |
| | | 6.2 Hz, 3H). | |
| 66 | 769.2 | H NMR (500 MHz, Chloroform-d) δ | 2 |
| | | 8.04-8.02 (m, 1H), 7.95 (d, J = 8.4 | |
| | | Hz, 1H), 7.81-7.75 (m, 1H), 7.45- | |
| | | 7.34 3(m, 3H), 7.24-7.16 (m, 3H), | |
| | | 6.83-6.77 (m, 1H), 5.31-5.30 (m, | |
| | | 2H), 4.63-4.58 (m, 1H), 4.51-4.40 | |
| | | (m, 2H), 4.39-4.30 (m, 2H), 3.89- | |
| | | 3.84 (m, 1H), 3.80-3.73 (m, 1H), | |
| | | 3.71 (s, 2H), 3.51-3.48 (m, 2H), | |
| | | 3.03-3.01 (m, 1H), 2.86-2.76 (m, | |
| | | 2H), 2.65-2.56 (m, 2H), 2.49-2.46 | |
| | | (m, 1H), 2.30-2.31 (m, 3H), 2.09- | |
| | | 1.94 (m, 4H), 1.37 (d, J = 6.2 Hz, | |
| | | 3H). | |
| 67 | 707.2 | ¹H NMR (500 MHz, Chloroform-d) δ | 2 |
| | | 8.16 (t, J = 5.6 Hz, 1H), 8.08 (d, J = | |
| | | 2.4 Hz, 1H), 7.82-7.78 (m, 1H), | |
| | | 7.36-7.32 (m, 3H), 7.24-7.17 (m, | |
| | | 3H), 6.80-6.77 (m, 1H), 5.31-5.30 | |
| | | (m, 2H), 4.39-4.30 (m, 2H), 4.23- | |
| | | 4.15 (m, 1H), 4.12-4.03 (m, 2H), | |
| | | 4.01-3.96 (m, 1H), 3.94-3.81 (m, | |
| | | 2H), 3.80-3.73 (m, 1H), 3.71 (s, | |
| | | 2H), 3.57-3.48 (m, 4H), 3.45-3.38 | |
| | | (m, 2H), 3.03-3.01 (m, 1H), 2.86- | |
| | | 2.75 (m, 2H), 2.67-2.56 (m, 2H), | |
| | | 2.30-2.22 (m, 2H), 2.09-1.94 (m, | |
| | | 4H). | |
| 68 | 783.3 | H NMR (500 MHz, Chloroform-d) δ | 2 |
| | | 8.04-8.02 (m, 1H), 7.95 (d, J = 8.4 | |
| | | Hz, 1H), 7.82-7.76 (m, 1H), 7.45- | |
| | | 7.35 (m, 3H), 7.24-7.15 (m, 3H), | |
| | | 6.82-6.80 (m, 1H), 5.31-5.30 (m, | |
| | | 2H), 4.63-4.58 (m, 1H), 4.51-4.40 | |
| | | (m, 2H), 4.39-4.30 (m, 2H), 3.89- | |
| | | 3.84 (m, 1H), 3.80-3.73 (m, 1H), | |
| | | 3.71 (s, 2H), 3.51-3.48 (m, 2H), | |
| | | 3.25 (s, 3H), 3.07-2.99 (m, 1H), | |
| | | 2.86-2.77 (m, 2H), 2.67-2.57 (m, | |
| | | 2H), 2.49-2.46 (m, 1H), 2.30-2.13 | |
| | | (m, 3H), 2.09-1.94 (m, 4H), 1.37 (d, | |
| | | J = 6.2 Hz, 3H). | |
| 69 | 767.3 | H NMR (500 MHz, Chloroform-d) δ | 2 |
| | | 8.04-8.02 (m, 1H), 7.95 (d, J = 8.4 | |
| | | Hz, 1H), 7.83-7.80 (m, 1H), 7.45- | |
| | | 7.34 (m, 3H), 7.22-7.15 (m, 3H), | |
| | | 6.87-6.79 (m, 1H), 5.31-5.30 (m, | |
| | | 2H), 4.63-4.58 (m, 1H), 4.51-4.40 | |
| | | (m, 2H), 4.39-4.32 (m, 1H), 4.32- | |
| | | 4.24 (m, 2H), 3.89-3.84 (m, 1H), | |
| | | 3.80-3.73 (m, 1H), 3.71 (s, 2H), | |
| | | 3.51-3.48 (m, 2H), 3.25 (s, 3H), 3.07- | |
| | | 2.99 (m, 1H), 2.86-2.78 (m, 2H), | |
| | | 2.67-2.58 (m, 2H), 2.49-2.46 (m, | |
| | | 1H), 2.30-2.13 (m, 3H), 2.09-1.94 | |
| | | (m, 4H). | |
| 70 | 691.2 | ¹H NMR (500 MHz, Chloroform-d) δ | 2 |
| | | 8.23 (t, J = 5.6 Hz, 1H), 8.08 (d, J = | |
| | | 2.4 Hz, 1H), 7.80-7.77 (m, 1H), | |
| | | 7.49 (t, J = 7.7 Hz, 1H), 7.42-7.34 | |
| | | (m, 2H), 7.22-7.17 (m, 3H), 6.86- | |
| | | 6.79 (m, 1H), 5.31-5.30 (m, 2H), 4.40- | |
| | | 4.32 (m, 2H), 4.32-4.24 (m, 1H), | |
| | | 4.23-4.15 (m, 1H), 4.06-3.93 (m, | |
| | | 2H), 3.89-3.84 (m, 1H), 3.80-3.73 | |
| | | (m, 1H), 3.71 (s, 2H), 3.64-3.50 (m, | |
| | | 3H), 3.41 (d, J = 5.5 Hz, 1H), 3.36- | |
| | | 3.27 (m, 1H), 3.03-3.01 (m, 1H), | |
| | | 2.86-2.76 (m, 2H), 2.67-2.59 (m, | |
| | | 2H), 2.30-2.22 (m, 2H), 2.09-1.94 | |
| | | (m, 5H), 1.86-1.77 (m, 1H). | |
| 71 | 781.3 | H NMR (500 MHz, Chloroform-d) δ | 3 |
| | | 8.04-8.02 (m, 1H), 7.95 (d, J = 8.4 | |
| | | Hz, 1H), 7.80-7.74 (m, 1H), 7.42- | |
| | | 7.34 (m, 3H), 7.25-7.17 (m, 3H), | |
| | | 6.86-6.78 (m, 1H), 5.31-5.30 (m, | |
| | | 2H), 4.63-4.58 (m, 1H), 4.51-4.40 | |
| | | (m, 2H), 4.39-4.30 (m, 2H), 3.89- | |
| | | 3.84 (m, 1H), 3.80-3.73 (m, 1H), | |
| | | 3.70 (s, 2H), 3.51-3.48 (m, 2H), | |
| | | 3.25 (s, 3H), 3.07-2.99 (m, 1H), | |
| | | 2.85-2.76 (m, 2H), 2.66-2.59 (m, | |
| | | 2H), 2.49-2.46 (m, 1H), 2.30-2.13 | |
| | | (m, 3H), 2.09-1.94 (m, 4H), 1.37 (d, | |
| | | J = 6.2 Hz, 3H). | |
| 72 | 753.2 | ¹H NMR (500 MHz, Chloroform-d) δ | 3 |
| | | 8.22-8.19 (m, 1H), 8.08 (d, J = 2.4 | |
| | | Hz, 1H), 7.83-7.78 (m, 1H), 7.49 (t, | |
| | | J = 7.7 Hz, 1H), 7.42-7.33 (m, 2H), | |
| | | 7.21-7.12 (m, 3H), 6.81-6.75 (m, | |
| | | 1H), 5.31-5.30 (m, 2H), 4.62-4.56 | |
| | | (m, 1H), 4.39-4.30 (m, 2H), 4.23- | |
| | | 4.15 (m, 1H), 4.04-3.96 (m, 1H), | |
| | | 3.98-3.83 (m, 3H), 3.86-3.81 (m, | |
| | | 1H), 3.83-3.73 (m, 1H), 3.71 (s, | |
| | | 2H), 3.03-3.01 (m, 1H), 2.85-2.75 | |
| | | (m, 2H), 2.74-2.52 (m, 4H), 2.30- | |
| | | 2.22 (m, 2H), 2.09-1.94 (m, 4H). | |
| 73 | 767.3 | H NMR (500 MHz, Chloroform-d) δ | 3 |
| | | 8.04-8.02 (m, 1H), 7.95 (d, J = 8.4 | |
| | | Hz, 1H), 7.83-7.77 (m, 1H), 7.42- | |
| | | 7.34 (m, 3H), 7.27-7.18 (m, 3H), | |
| | | 6.83-6.73 (m, 1H), 5.31-5.30 (m, | |
| | | 2H), 4.63-4.58 (m, 1H), 4.51-4.40 | |
| | | (m, 2H), 4.39-4.30 (m, 2H), 3.89- | |
| | | 3.84 (m, 1H), 3.80-3.73 (m, 1H), | |
| | | 3.71 (s, 2H), 3.51-3.48 (m, 2H), | |
| | | 3.03-3.01 (m, 1H), 2.85-2.76 (m, | |
| | | 2H), 2.66-2.57 (m, 2H), 2.49-2.46 | |
| | | (m, 1H), 2.30-2.13 (m, 3H), 2.09- | |
| | | 1.94 (m, 4H), 1.37 (d, J = 6.2 Hz, | |
| | | 3H). | |
| 74 | 790.3 | ¹H NMR (500 MHz, Chloroform-d) δ | 2 |
| | | 7.99-7.96 (m, 1H), 7.95 (d, J = 8.4 | |
| | | Hz, 1H), 7.83-7.76 (m, 1H), 7.49 (t, | |
| | | J = 7.7 Hz, 1H), 7.42-7.35 (m, 2H), | |
| | | 7.21-7.12 (m, 3H), 6.81-6.71 (m, | |
| | | 1H), 5.28 (d, J = 1.2 Hz, 2H), 4.62- | |
| | | 4.53 (m, 1H), 4.46 (t, J = 5.8 Hz, | |
| | | 1H), 4.39-4.30 (m, 2H), 4.23- | |
| | | 4.15 (m, 1H), 3.91-3.83 (m, 1H), | |
| | | 3.78-3.71 (m, 1H), 3.68 (d, J = 3.8 | |
| | | Hz, 5H), 3.63-3.51 (m, 2H), 3.38- | |
| | | 3.29 (m, 1H), 3.03-3.01 (m, 1H), | |
| | | 2.86-2.76 (m, 2H), 2.68-2.57 (m, | |
| | | 2H), 2.36-2.31 (m, 2H), 2.25 (s, | |
| | | 6H), 2.14-1.94 (m, 6H), 1.37 (d, J = | |
| | | 6.2 Hz, 3H). | |
| 75 | 644.1 | ¹H NMR (500 MHz, Chloroform-d) δ | 2 |
| | | 8.91 (s, 1H), 8.41 (s, 1H), 8.05 (d, J = | |
| | | 2.0 Hz, 1H), 7.86-7.81 (m, 1H), | |
| | | 7.49-7.37 (m, 2H), 7.33-7.28 (m, | |
| | | 2H), 7.25-7.13 (m, 3H), 6.82-6.72 | |
| | | (m, 1H), 6.79-6.73 (m, 2H), 5.31- | |
| | | 5.30 (m, 2H), 4.41-4.38 (m, 2H), 3.80- | |
| | | 3.73 (m, 4H), 3.32 (s, 3H), 3.03- | |
| | | 3.01 (m, 1H), 2.86-2.78 (m, 2H), | |
| | | 2.68-2.59 (m, 2H), 2.09-1.94 (m, | |
| | | 4H). | |
| 76 | 710.2 | ¹H NMR (500 MHz, Chloroform-d) δ | 2 |
| | | 8.92 (t, J = 6.3 Hz, 1H), 8.08 (d, J = | |
| | | 2.2 Hz, 1H), 7.97 (d, J = 2.1 Hz, 1H), | |
| | | 7.80-7.76 (m, 1H), 7.45-7.41 (m, | |
| | | 4H), 7.24-7.17 (m, 4H), 6.83-6.75 | |
| | | (m, 1H), 5.31-5.30 (m, 2H), 4.56 (d, J = | |
| | | 6.3 Hz, 2H), 4.39-4.30 (m, 2H), | |
| | | 4.23-4.15 (m, 1H), 3.89-3.84 (m, | |
| | | 1H), 3.80-3.73 (m, 1H), 3.71 (s, | |
| | | 2H), 3.03-3.01 (m, 1H), 2.87-2.78 | |
| | | (m, 2H), 2.68-2.58 (m, 2H), 2.30- | |
| | | 2.22 (m, 2H), 2.09-1.94 (m, 4H). | |
| 77 | 691.2 | ¹H NMR (500 MHz, Chloroform-d) δ | 2 |
| | | 8.23 (t, J = 5.6 Hz, 1H), 8.08 (d, J = | |
| | | 2.4 Hz, 1H), 7.82-7.78 (m, 1H), | |
| | | 7.49 (t, J = 7.7 Hz, 1H), 7.42-7.34 | |
| | | (m, 2H), 7.21-7.13 (m, 3H), 6.85- | |
| | | 6.77 (m, 1H), 5.31-5.30 (m, 2H), 4.40- | |
| | | 4.32 (m, 2H), 4.32-4.24 (m, 1H), | |
| | | 4.23-4.15 (m, 1H), 4.06-3.93 (m, | |
| | | 2H), 3.89-3.84 (m, 1H), 3.80-3.73 | |
| | | (m, 1H), 3.71 (s, 2H), 3.64-3.50 (m, | |
| | | 3H), 3.41 (d, J = 5.5 Hz, 1H), 3.36- | |
| | | 3.27 (m, 1H), 3.03-3.01 (m, 1H), | |
| | | 2.87-2.76 (m, 2H), 2.66-2.58 (m, | |
| | | 2H), 2.30-2.22 (m, 2H), 2.09-1.94 | |
| | | (m, 5H), 1.86-1.77 (m, 1H). | |
| 78 | 682.1 | ¹H NMR (500 MHz, Chloroform-d) δ | 2 |
| | | 9.02 (s, 1H), 8.39 (s, 1H), 8.13 (d, J = | |
| | | 2.1 Hz, 1H), 7.82-7.80 (m, 1H), | |
| | | 7.48-7.37 (m, 3H), 7.21-7.11 (m, | |
| | | 3H), 6.86-6.79 (m, 1H), 6.48 (d, J = | |
| | | 8.1 Hz, 1H), 6.40 (d, J = 7.9 Hz, 1H), | |
| | | 5.31-5.30 (m, 2H), 4.39-4.30 (m, | |
| | | 2H), 4.23-4.15 (m, 1H), 3.91-3.83 | |
| | | (m, 1H), 3.78-3.74 (m, 1H), 3.71 (s, | |
| | | 2H), 3.03-3.01 (m, 1H), 2.85-2.76 | |
| | | (m, 2H), 2.66-2.59 (m, 2H), 2.36- | |
| | | 2.31 (m, 1H), 2.09-1.94 (m, 5H). | |
| 79 | 695.2 | ¹H NMR (500 MHz, Chloroform-d) δ | 2 |
| | | 8.74 (d, J = 1.6 Hz, 1H), 8.67 (d, J = | |
| | | 2.0 Hz, 1H), 8.05 (d, J = 2.1 Hz, 1H), | |
| | | 7.86-7.81 (m, 1H), 7.49-7.39 (m, | |
| | | 2H), 7.21-7.14 (m, 3H), 6.86-6.74 | |
| | | (m, 1H), 5.28 (d, J = 1.2 Hz, 2H), | |
| | | 4.39-4.30 (m, 2H), 4.23-4.15 (m, | |
| | | 1H), 3.89-3.84 (m, 1H), 3.80-3.73 | |
| | | (m, 1H), 3.71 (s, 2H), 3.03-3.01 (m, | |
| | | 1H), 2.94 (s, 3H), 2.85-2.75 (m, | |
| | | 2H), 2.65-2.59 (m, 2H), 2.30-2.22 | |
| | | (m, 2H), 2.09-1.95 (m, 4H). | |
| 80 | 694.2 | ¹H NMR (500 MHz, Chloroform-d) δ | 2 |
| | | 8.05 (d, J = 2.4 Hz, 1H), 7.86-7.80 | |
| | | (m, 2H), 7.52 (d, J = 2.1 Hz, 1H), | |
| | | 7.45-7.39 (m, 2H), 7.21-7.12 (m, | |
| | | 3H), 6.83-6.73 (m, 1H), 5.31-5.30 | |
| | | (m, 2H), 4.39-4.30 (m, 2H), 4.23- | |
| | | 4.15 (m, 1H), 3.89-3.84 (m, 1H), | |
| | | 3.80-3.73 (m, 1H), 3.71 (s, 2H), | |
| | | 3.03-3.01 (m, 1H), 2.85-2.76 (m, | |
| | | 2H), 2.68 (s, 3H), 2.65-2.60 (m, | |
| | | 2H), 2.30-2.22 (m, 2H), 2.09-1.94 | |
| | | (m, 4H). | |
| 81 | 710.3 | ¹H NMR (500 MHz, DMSO-d₆) δ | 2 |
| | | 12.66 (s, 1H), 10.49 (s, 1H), 8.23 (d, | |
| | | J = 2.2 Hz, 1H), 8.12 (d, J = 2.1 Hz, | |
| | | 1H), 7.96-7.92 (m, 1H), 7.70-7.62 | |
| | | (m, 2H), 7.53-7.47 (m, 2H), 7.30- | |
| | | 7.17 (m, 3H), 6.76-6.75 (m, 1H), | |
| | | 5.35-5.34 (m, 2H), 4.33-4.28 (m, | |
| | | 2H), 4.13 (s, 3H), 4.06-4.01 (m, 1H), | |
| | | 3.87-3.79 (m, 1H), 3.78-3.74 (m, | |
| | | 1H), 3.69 (s, 2H), 3.11-2.99 (m, | |
| | | 3H), 2.87-2.78 (m, 2H), 2.19-2.10 | |
| | | (m, 2H), 2.03-1.86 (m, 4H). | |
| 82 | 705.2 | ¹H NMR (500 MHz, Chloroform-d) δ | 2 |
| | | 8.21 (d, J = 2.1 Hz, 1H), 8.05 (d, J = | |
| | | 2.4 Hz, 1H), 7.83 (dd, J = 8.7, 2.1 | |
| | | Hz, 1H), 7.70-7.67 (m, 1H), 7.60 (d, | |
| | | J = 7.9 Hz, 1H), 7.49 (t, J = 7.7 Hz, | |
| | | 1H), 7.42-7.38 (m, 2H), 7.21-7.13 | |
| | | (m, 3H), 6.81-6.75 (m, 1H), 5.31- | |
| | | 5.30 (m, 2H), 4.35-4.17 (m, 3H), 3.89- | |
| | | 3.84 (m, 1H), 3.80-3.73 (m, 1H), | |
| | | 3.71 (s, 2H), 3.03-3.01 (m, 1H), | |
| | | 2.83-2.74 (m, 2H), 2.69-2.60 (m, | |
| | | 2H), 2.30-2.22 (m, 2H), 2.09-1.94 | |
| | | (m, 4H). | |
| 83 | 722.3 | ¹H NMR (500 MHz, Chloroform-d) δ | 2 |
| | | 11.76 (s, 1H), 10.09 (s, 1H), 8.04 (dd, | |
| | | J = 10.5, 2.1 Hz, 2H), 7.83-7.54 (m, | |
| | | 3H), 7.49 (t, J = 7.7 Hz, 1H), 7.42- | |
| | | 7.38 (m, 2H), 7.21-7.13 (m, 3H), | |
| | | 6.81-6.75 (m, 1H), 5.29 (d, J = 1.2 | |
| | | Hz, 2H), 4.42-4.38 (m, 1H), 4.32- | |
| | | 4.24 (m, 1H), 4.17 (dd, J = 15.6, 5.3 | |
| | | Hz, 1H), 3.89-3.84 (m, 1H), 3.80- | |
| | | 3.73 (m, 1H), 3.71 (s, 2H), 3.11- | |
| | | 3.03 (m, 1H), 3.06-2.60 (m, 5H), | |
| | | 2.32-1.92 (m, 6H), 1.66 (d, J = 6.6 | |
| | | Hz, 3H), 1.60 (s, 1H). | |
| 84 | 708.2 | ¹H NMR (500 MHz, DMSO-d₆) δ | 2 |
| | | 11.78 (s, 1H), 10.88 (s, 1H), 8.23 (d, | |
| | | J = 2.2 Hz, 1H), 8.14 (s, 1H), 7.96- | |
| | | 7.92 (m, 1H), 7.76 (d, J = 2.1 Hz, | |
| | | 1H), 7.62 (dd, J = 7.8, 2.0 Hz, 1H), | |
| | | 7.53-7.51 (m, 1H), 7.46-7.40 (m, | |
| | | 3H), 7.30-7.17 (m, 3H), 6.76-6.75 | |
| | | (m, 1H), 5.35-5.34 (m, 2H), 4.43- | |
| | | 4.04 (m, 3H), 3.87-3.79 (m, 1H), | |
| | | 3.78-3.74 (m, 1H), 3.69 (s, 2H), | |
| | | 3.11-2.78 (m, 5H), 2.19-2.10 (m, | |
| | | 2H), 2.03-1.86 (m, 4H). | |
| 85 | 696.2 | ¹H NMR (500 MHz, Chloroform-d) δ | 2 |
| | | 12.33 (s, 1H), 10.12 (s, 1H), 10.32 (s, | |
| | | 1H), 8.27 (d, J = 2.0 Hz, 1H), 7.90 (d, | |
| | | J = 2.4 Hz, 1H), 7.79-7.77 (m, 1H), | |
| | | 7.55-7.44 (m, 4H), 7.42-7.36 (m, | |
| | | 1H), 7.21-7.13 (m, 3H), 6.81-6.75 | |
| | | (m, 1H), 5.31-5.30 (m, 2H), 4.42- | |
| | | 4.38 (m, 1H), 4.32-4.02 (m, 2H), | |
| | | 3.89-3.73 (m, 2H), 3.69 (s, 2H), 3.03- | |
| | | 3.01 (m, 1H), 2.83-2.60 (m, 4H), | |
| | | 2.30-2.22 (m, 2H), 2.03-1.94 (m, | |
| | | 4H). | |
| 86 | 721.3 | ¹H NMR (500 MHz, Chloroform-d) δ | 2 |
| | | 12.64 (s, 1H), 10.54 (s, 1H), 9.09 (s, | |
| | | 1H), 8.05 (d, J = 2.1 Hz, 1H), 7.83 | |
| | | (dd, J = 8.7, 2.1 Hz, 1H), 7.68 (d, J = | |
| | | 1.8 Hz, 1H), 7.60 (dd, J = 7.8, 2.1 | |
| | | Hz, 1H), 7.49 (t, J = 7.7 Hz, 1H), | |
| | | 7.42-7.38 (m, 2H), 7.21-7.13 (m, | |
| | | 3H), 6.81-6.75 (m, 1H), 5.31-5.30 | |
| | | (m, 2H), 4.42-4.38 (m, 1H), 4.32- | |
| | | 4.17 (m, 2H), 3.89-3.73 (m, 2H), | |
| | | 3.71 (s, 2H), 3.07-2.74 (m, 3H), | |
| | | 2.69-2.60 (m, 2H), 2.32-2.11 (m, | |
| | | 4H), 2.09-1.94 (m, 4H), 1.70-1.65 | |
| | | (m, 2H). | |
| 87 | 680.3 | ¹H NMR (500 MHz, DMSO-d₆) δ | 2 |
| | | 8.21 (d, J = 1.8 Hz, 2H), 8.04 (t, J = | |
| | | 2.1 Hz, 1H), 7.91-7.88 (m, 1H), | |
| | | 7.69-7.67 (m, 1H), 7.55-7.40 (m, | |
| | | 4H), 7.30-7.17 (m, 3H), 6.76-6.75 | |
| | | (m, 1H), 5.35-5.34 (m, 2H), 4.46- | |
| | | 4.21 (m, 2H), 4.06-4.01 (m, 1H), 3.90- | |
| | | 3.68 (m, 4H), 3.11-2.99 (m, 3H), | |
| | | 2.87-2.78 (m, 2H), 2.33-1.92 (m, | |
| | | 6H). | |
| 88 | 694.3 | ¹H NMR (500 MHz, Chloroform-d) δ | 2 |
| | | 10.32 (s, 1H), 8.05 (d, J = 2.0 Hz, | |
| | | 1H), 7.88 (d, J = 2.2 Hz, 1H), 7.83 | |
| | | (dd, J = 8.7, 2.1 Hz, 1H), 7.65 (dd, | |
| | | J = 7.8, 2.1 Hz, 1H), 7.49 (t, J = 7.7 | |
| | | Hz, 1H), 7.44-7.36 (m, 2H), 7.21- | |
| | | 7.13 (m, 3H), 6.81-6.75 (m, 1H), 5.29 | |
| | | (d, J = 1.2 Hz, 2H), 4.35-4.24 (m, | |
| | | 2H), 4.17-3.70 (m, 3H), 3.71 (s, | |
| | | 2H), 3.03-3.01 (m, 1H), 2.90 (s, | |
| | | 2H), 2.83-2.60 (m, 4H), 2.39-1.94 | |
| | | (m, 6H). | |
| 89 | 682.2 | ¹H NMR (500 MHz, Chloroform-d) δ | 2 |
| | | 11.2 2 (s, 1H), 8.95 (s, 1H), 8.41 (d, J = | |
| | | 8.1 Hz, 1H), 8.05 (d, J = 2.2 Hz, | |
| | | 1H), 7.85 (dd, J = 8.7, 2.1 Hz, 1H), | |
| | | 7.49 (t, J = 7.7 Hz, 1H), 7.42-7.38 | |
| | | (m, 2H), 7.19 (dd, J = 8.0, 2.2 Hz, | |
| | | 1H), 7.18-7.13 (m, 2H), 6.81-6.75 | |
| | | (m, 1H), 5.31-5.30 (m, 2H), 4.33- | |
| | | 4.19 (m, 3H), 3.91-3.83 (m, 1H), | |
| | | 3.78-3.74 (m, 1H), 3.71 (s, 2H), | |
| | | 3.07-2.60 (m, 5H), 2.39-1.92 (m, | |
| | | 6H). | |
| 90 | 696.2 | ¹H NMR (500 MHz, Chloroform-d) δ | 2 |
| | | 9.39 (s, 1H), 8.05 (d, J = 2.0 Hz, 1H), | |
| | | 7.83 (dd, J = 8.7, 2.1 Hz, 1H), 7.58 | |
| | | (dd, J = 8.1, 1.8 Hz, 1H), 7.49 (t, J = | |
| | | 7.7 Hz, 1H), 7.42-7.38 (m, 2H), | |
| | | 7.34 (d, J = 2.0 Hz, 1H), 7.21-7.13 | |
| | | (m, 3H), 6.81-6.75 (m, 1H), 5.31- | |
| | | 5.30 (m, 2H), 4.35-4.17 (m, 3H), | |
| | | 3.91-3.83 (m, 1H), 3.78-3.74 (m, | |
| | | 1H), 3.71 (s, 1H), 3.07-2.60 (m, | |
| | | 5H), 2.39-1.94 (m, 6H). | |
| 91 | 710.3 | ¹H NMR (500 MHz, Chloroform-d) δ | 2 |
| | | 10.11 (s, 1H), 9.46 (s, 1H), 8.07- | |
| | | 8.03 (m, 1H), 7.83 (dd, J = 8.7, 2.1 | |
| | | Hz, 1H), 7.59 (dd, J = 8.2, 2.1 Hz, | |
| | | 1H), 7.49 (t, J = 7.9 Hz, 1H), 7.42- | |
| | | 7.38 (m, 2H), 7.32 (d, J = 2.1 Hz, | |
| | | 1H), 7.27 (d, J = 8.0 Hz, 1H), 7.20- | |
| | | 7.17 (m, 1H), 7.18-7.13 (m, 2H), | |
| | | 6.79 (dd, J = 7.8, 1.1 Hz, 1H), 5.31- | |
| | | 5.30 (m, 2H), 4.35-4.17 (m, 3H), | |
| | | 3.91-3.70 (m, 2H), 3.71 (s, 2H), 3.46 | |
| | | (s, 2H), 3.07-2.99-2.60 (m, 5H), | |
| | | 2.93-1.92 (m, 6H). | |
| 92 | 714.2 | ¹H NMR (500 MHz, DMSO-d₆) δ | 2 |
| | | 10.73 (s, 1H), 10.64 (s, 1H), 10.15 (s, | |
| | | 1H), 8.45 (d, J = 5.0 Hz, 1H), 8.23 (d, | |
| | | J = 1.8 Hz, 1H), 7.93 (dd, J = 8.7, | |
| | | 1.8 Hz, 1H), 7.70 (d, J = 8.0 Hz, 1H), | |
| | | 7.50-7.40 (m, 3H), 7.30-7.21 (m, | |
| | | 2H), 7.19-7.18 (m, 1H), 6.81-6.78 | |
| | | (m, 1H), 5.35-5.34 (m, 2H), 4.35 (dd, | |
| | | J = 15.5, 5.3 Hz, 1H), 4.30-3.92 (m, | |
| | | 3H), 3.75-3.69 (m, 1H), 3.69 (s, | |
| | | 2H), 3.11-2.99 (m, 3H), 2.87-2.78 | |
| | | (m, 2H), 2.33-1.90 (m, 7H). | |
| 93 | 697.2 | ¹H NMR (500 MHz, DMSO-d₆) δ | 2 |
| | | 11.15 (s, 1H), 10.96 (s, 1H), 10.74 (s, | |
| | | 1H), 8.23-8.16 (m, 1H), 7.93 (dd, J = | |
| | | 8.8, 2.1 Hz, 1H), 7.54 (d, J = 7.9 | |
| | | Hz, 1H), 7.50-7.40 (m, 3H), 7.35- | |
| | | 7.28 (m, 2H), 7.21 (dd, J = 7.9, 2.2 | |
| | | Hz, 1H), 7.19-7.18 (m, 1H), 6.81- | |
| | | 6.78 (m, 1H), 5.35-5.34 (m, 2H), | |
| | | 4.35-3.73 (m, 5H), 3.69 (s, 2H), 3.11- | |
| | | 2.78 (m, 5H), 2.30-1.90 (m, 7H). | |
| 94 | 694.3 | ¹H NMR (500 MHz, DMSO-d₆) δ | 2 |
| | | 10.53 (s, 1H), 8.23 (d, J = 1.8 Hz, | |
| | | 1H), 8.08 (d, J = 1.8 Hz, 1H), 7.98 (d, | |
| | | J = 8.7 Hz, 1H), 7.97-7.94 (m, 1H), | |
| | | 7.55 (dd, J = 8.6, 2.1 Hz, 1H), 7.50- | |
| | | 7.40 (m, 3H), 7.30-7.21 (m, 2H), | |
| | | 7.19-7.18 (m, 1H), 6.81-6.78 (m, | |
| | | 1H), 5.35-5.34 (m, 2H), 4.35-4.26 | |
| | | (m, 2H), 4.04-3.67 (m, 3H), 3.69 (s, | |
| | | 2H), 3.11-2.78 (m, 5H), 2.34-1.90 | |
| | | (m, 6H). | |
| 95 | 711.2 | ¹H NMR (500 MHz, DMSO-d₆) δ | 2 |
| | | 10.11 (s, 1H), 8.46-8.42 (m, 1H), | |
| | | 8.23 (d, J = 1.8 Hz, 1H), 7.97-7.94 | |
| | | (m, 1H), 7.83-7.80 (m, 1H), 7.75- | |
| | | 7.74 (m, 1H), 7.53-7.42 (m, 2H), | |
| | | 7.42 (d, J = 8.7 Hz, 1H), 7.30-7.21 | |
| | | (m, 2H), 7.19-7.18 (m, 1H), 6.81- | |
| | | 6.78 (m, 1H), 5.35-5.34 (m, 2H), 4.35- | |
| | | 4.26 (m, 2H), 4.04-3.67 (m, 3H), | |
| | | 3.69 (s, 2H), 3.11-2.82 (m, 5H), | |
| | | 2.36-1.90 (m, 7H). | |
| 96 | 680.3 | ¹H NMR (500 MHz, DMSO-d₆) δ | 2 |
| | | 10.24 (s, 1H), 8.44 (d, J = 6.9 Hz, | |
| | | 1H), 8.28-8.23 (m, 1H), 8.23 (d, J = | |
| | | 1.8 Hz, 1H), 8.06-8.02 (m, 1H), | |
| | | 7.93 (dd, J = 8.7, 1.8 Hz, 1H), 7.53- | |
| | | 7.49 (m, 3H), 7.43-7.32 (m, 1H), | |
| | | 7.30-7.21 (m, 2H), 7.19-7.18 (m, | |
| | | 1H), 6.80-6.77 (m, 2H), 5.35-5.34 | |
| | | (m, 2H), 4.35-4.04 (m, 3H), 3.88- | |
| | | 3.70 (m, 2H), 3.69 (s, 2H), 3.11- | |
| | | 2.99 (m, 3H), 2.87-2.78 (m, 2H), | |
| | | 2.32-1.90 (m, 6H). | |
| 97 | 714.2 | ¹H NMR (500 MHz, DMSO-d₆) δ | 2 |
| | | 10.78 (s, 1H), 8.49 (d, J = 6.9 Hz, | |
| | | 1H), 8.21-8.19 (m, 2H), 8.04 (s, 1H), | |
| | | 7.93 (dd, J = 8.7, 1.8 Hz, 1H), 7.62- | |
| | | 7.60 (m, 1H), 7.53-7.42 (m, 2H), | |
| | | 7.42 (d, J = 8.7 Hz, 1H), 7.30-7.21 | |
| | | (m, 2H), 7.19-7.18 (m, 1H), 6.81- | |
| | | 6.78 (m, 1H), 5.35-5.34 (m, 2H), | |
| | | 4.41-4.37 (m, 1H), 4.25-4.18 (m, | |
| | | 1H), 4.04 (dd, J = 15.6, 3.9 Hz, 1H), | |
| | | 3.88-3.80 (m, 1H), 3.77-3.70 (m, | |
| | | 1H), 3.69 (s, 2H), 3.11-2.78 (m, | |
| | | 5H), 2.32-1.90 (m, 6H). | |
| 98 | 697.2 | ¹H NMR (500 MHz, DMSO-d₆) δ | 2 |
| | | 10.09 (s, 1H), 9.35 (s, 1H), 8.42- | |
| | | 8.38 (m, 1H), 8.23 (d, J = 1.8 Hz, | |
| | | 1H), 7.97-7.94 (m, 1H), 7.81-7.76 | |
| | | (m, 1H), 7.71 (dd, J = 7.8, 2.1 Hz, | |
| | | 1H), 7.53-7.42 (m, 2H), 7.42 (d, J = | |
| | | 8.7 Hz, 1H), 7.30-7.21 (m, 2H), | |
| | | 7.19-7.18 (m, 1H), 6.81-6.78 (m, | |
| | | 1H), 5.35-5.34 (m, 2H), 4.35-4.26 | |
| | | (m, 2H), 4.04-3.70 (m, 3H), 3.69 (s, | |
| | | 2H), 3.11-2.78 (m, 5H), 2.33-1.90 | |
| | | (m, 6H). | |
| 99 | 694.3 | ¹H NMR (500 MHz, DMSO-d₆) δ | 2 |
| | | 10.18 (s, 1H), 8.22-8.17 (m, 2H), | |
| | | 8.11-8.10 (m, 1H), 7.97-7.94 (m, | |
| | | 1H), 7.71 (dd, J = 7.8, 2.1 Hz, 1H), | |
| | | 7.65 (d, J = 7.6 Hz, 1H), 7.53-7.42 | |
| | | (m, 2H), 7.42 (d, J = 8.7 Hz, 1H), | |
| | | 7.30-7.21 (m, 2H), 7.19-7.18 (m, | |
| | | 1H), 6.81-6.78 (m, 1H), 5.35-5.34 | |
| | | (m, 2H), 4.35-4.26 (m, 2H), 4.09- | |
| | | 4.02 (m, 1H), 3.92 (s, 3H), 3.87- | |
| | | 3.74 (m, 2H), 3.69 (s, 2H), 3.11- | |
| | | 2.78 (m, 5H), 2.30-1.90 (m, 6H). | |
| 100 | 708.3 | ¹H NMR (500 MHz, DMSO-d₆) δ | 2 |
| | | 8.13 (d, J = 1.8 Hz, 1H), 7.97-7.94 | |
| | | (m, 1H), 7.70 (d, J = 2.1 Hz, 1H), | |
| | | 7.53-7.45 (m, 2H), 7.49-7.40 (m, | |
| | | 2H), 7.30-7.21 (m, 2H), 7.19-7.18 | |
| | | (m, 1H), 6.81-6.78 (m, 1H), 5.35- | |
| | | 5.34 (m, 2H), 4.35-4.26 (m, 2H), | |
| | | 4.09-4.02 (m, 1H), 3.90-3.82 (m, | |
| | | 1H), 3.75-3.69 (m, 1H), 3.69 (s, | |
| | | 2H), 3.11-2.78 (m, 5H), 2.73 (s, | |
| | | 2H), 2.33-1.92 (m, 6H). | |
| 101 | 704.3 | ¹H NMR (500 MHz, Chloroform-d) δ | 2 |
| | | 9.19 (s, 1H), 8.05 (d, J = 2.1 Hz, 1H), | |
| | | 7.83-7.82 (m, 1H), 7.66 (d, J = 1.8 | |
| | | Hz, 1H), 7.61-7.58 (m, 1H), 7.49 (t, J = | |
| | | 7.7 Hz, 1H), 7.42-7.38 (m, 2H), | |
| | | 7.21-7.13 (m, 3H), 6.81-6.75 (m, | |
| | | 1H), 5.31-5.30 (m, 2H), 4.42-4.38 | |
| | | (m, 1H), 4.32-4.17 (m, 2H), 3.89- | |
| | | 3.73 (m, 2H), 3.71 (s, 2H), 3.07- | |
| | | 2.74 (m, 3H), 2.69-2.60 (m, 2H), | |
| | | 2.32-2.11 (m, 4H), 2.09-1.94 (m, | |
| | | 4H), 1.70-1.65 (m, 2H). | |
| 102 | 698.2 | ¹H NMR (500 MHz, DMSO-d₆) δ | 2 |
| | | 8.21 (dd, J = 12.0, 2.0 Hz, 2H), 7.92 | |
| | | (t, J = 2.0 Hz, 1H), 7.93 (dd, J = 8.7, | |
| | | 2.1 Hz, 1H), 7.65 (dd, J = 8.0, 2.1 | |
| | | Hz, 1H), 7.53-7.42 (m, 2H), 7.42 | |
| | | (d, J = 8.7 Hz, 1H), 7.30-7.21 (m, | |
| | | 2H), 7.19-7.18 (m, 1H), 6.81-6.78 | |
| | | (m, 1H), 5.35-5.34 (m, 2H), 4.35- | |
| | | 4.04 (m, 3H), 3.87-3.70 (m, 2H), | |
| | | 3.69 (s, 2H), 3.11-2.99 (m, 3H), | |
| | | 2.87-2.78 (m, 2H), 2.30-1.90 (m, | |
| | | 6H). | |
| 103 | 712.1 | ¹H NMR (500 MHz, DMSO-d₆) δ | 2 |
| | | 12.44 (s, 1H), 10.82 (s, 1H), 8.23 (d, | |
| | | J = 1.8 Hz, 1H), 7.97-7.94 (m, 1H), | |
| | | 7.82 (d, J = 2.2 Hz, 1H), 7.55 (dd, J = | |
| | | 8.0, 2.1 Hz, 1H), 7.53-7.40 (m, 3H), | |
| | | 7.30-7.21 (m, 2H), 7.19-7.18 (m, | |
| | | 1H), 6.81-6.78 (m, 1H), 5.35-5.34 | |
| | | (m, 2H), 4.35-4.26 (m, 2H), 4.04- | |
| | | 3.67 (m, 3H), 3.69 (s, 2H), 3.11- | |
| | | 2.82 (m, 5H), 2.36-1.90 (m, 7H). | |
| 104 | 739.3 | ¹H NMR (500 MHz, DMSO-d₆) δ | 2 |
| | | 10.17 (s, 1H), 9.97 (s, 1H), 8.23 (d, J = | |
| | | 2.1 Hz, 1H), 7.93 (dd, J = 8.7, 2.1 | |
| | | Hz, 1H), 7.63-7.56 (m, 2H), 7.53- | |
| | | 7.42 (m, 2H), 7.42 (d, J = 8.7 Hz, | |
| | | 1H), 7.30-7.21 (m, 2H), 7.19-7.18 | |
| | | (m, 1H), 6.81-6.78 (m, 1H), 5.35- | |
| | | 5.34 (m, 2H), 4.35-4.26 (m, 2H), | |
| | | 4.04-3.70 (m, 3H), 3.69 (s, 2H), | |
| | | 3.11-2.78 (m, 5H), 2.30-2.18 (m, | |
| | | 2H), 2.21-1.90 (m, 6H), 1.73-1.66 | |
| | | (m, 1H), 1.69-1.62 (m, 1H). | |
| 105 | 735.3 | ¹H NMR (500 MHz, DMSO-d₆) δ | 2 |
| | | 10.01 (s, 1H), 9.93 (s, 1H), 8.23 (d, J = | |
| | | 2.1 Hz, 1H), 7.93 (dd, J = 8.7, 2.1 | |
| | | Hz, 1H), 7.65 (d, J = 2.1 Hz, 1H), | |
| | | 7.55 (d, J = 2.0 Hz, 1H), 7.53-7.42 | |
| | | (m, 2H), 7.42 (d, J = 8.7 Hz, 1H), | |
| | | 7.30-7.21 (m, 2H), 7.19-7.18 (m, | |
| | | 1H), 6.81-6.78 (m, 1H), 5.35-5.34 | |
| | | (m, 2H), 4.35-4.26 (m, 2H), 4.04- | |
| | | 3.70 (m, 3H), 3.69 (s, 2H), 3.11-2.78 | |
| | | (m, 5H), 2.30-2.20 (m, 5H), 2.17- | |
| | | 1.90 (m, 6H), 1.72-1.61 (m, 2H). | |
| 106 | 762.3 | ¹H NMR (500 MHz, DMSO-d₆) δ | 2 |
| | | 8.23 (d, J = 1.8 Hz, 1H), 8.08 (d, J = | |
| | | 2.1 Hz, 1H), 7.97-7.94 (m, 1H), 7.86 | |
| | | (d, J = 2.1 Hz, 1H), 7.50-7.40 (m, | |
| | | 3H), 7.30-7.21 (m, 2H), 7.19-7.18 | |
| | | (m, 1H), 6.81-6.78 (m, 1H), 5.35- | |
| | | 5.34 (m, 2H), 4.35-4.26 (m, 2H), | |
| | | 4.01-3.77 (m, 3H), 3.69 (s, 2H), 3.11- | |
| | | 2.99 (m, 3H), 2.87-2.78 (m, 2H), | |
| | | 2.36-1.90 (m, 6H). | |
| 107 | 789.3 | ¹H NMR (500 MHz, DMSO-d₆) δ | 2 |
| | | 10.02 (s, 1H), 9.94 (s, 1H), 8.23 (d, J = | |
| | | 2.1 Hz, 1H), 8.11 (d, J = 2.1 Hz, | |
| | | 1H), 7.93 (dd, J = 8.7, 2.1 Hz, 1H), | |
| | | 7.62 (d, J = 2.1 Hz, 1H), 7.53-7.42 | |
| | | (m, 2H), 7.42 (d, J = 8.7 Hz, 1H), | |
| | | 7.30-7.21 (m, 2H), 7.19-7.18 (m, | |
| | | 1H), 6.81-6.78 (m, 1H), 5.35-5.34 | |
| | | (m, 2H), 4.35-4.26 (m, 2H), 4.04- | |
| | | 3.70 (m, 3H), 3.69 (s, 2H), 3.11- | |
| | | 2.99 (m, 3H), 2.87-2.78 (m, 2H), | |
| | | 2.30-1.90 (m, 8H), 1.72-1.61 (m, | |
| | | 2H). | |
| 108 | 748.2 | ¹H NMR (500 MHz, DMSO-d₆) δ | 2 |
| | | 8.19 (dd, J = 12.0, 2.1 Hz, 1H), 8.04 | |
| | | (d, J = 2.2 Hz, 1H), 7.99 (t, J = 2.0 | |
| | | Hz, 1H), 7.96-7.92 (m, 1H), 7.53- | |
| | | 7.42 (m, 2H), 7.42 (d, J = 8.8 Hz, | |
| | | 1H), 7.30-7.17 (m, 3H), 6.76-6.75 | |
| | | (m, 1H), 5.35-5.34 (m, 2H), 4.42- | |
| | | 4.38 (m, 1H), 4.32-4.28 (m, 1H), | |
| | | 4.04-3.70 (m, 3H), 3.69 (s, 2H), | |
| | | 3.11-2.99 (m, 3H), 2.87-2.78 (m, | |
| | | 2H), 2.30-1.90 (m, 6H). | |
| 109 | 708.3 | ¹H NMR (500 MHz, DMSO-d₆) δ | 2 |
| | | 10.11 (s, 1H), 8.23 (d, J = 2.1 Hz, | |
| | | 1H), 7.97-7.93 (m, 1H), 7.91-7.88 | |
| | | (m, 1H), 7.62 (dd, J = 8.4, 2.1 Hz, | |
| | | 1H), 7.56-7.40 (m, 4H), 7.32-7.24 | |
| | | (m, 2H), 1H), 6.76-6.75 (m, 1H), | |
| | | 5.35-5.34 (m, 2H), 4.35-4.04 (m, | |
| | | 3H), 3.90 (s, 3H), 3.87-3.74 (m, | |
| | | 2H), 3.69 (s, 2H), 3.11-2.78 (m, | |
| | | 5H), 2.30-1.90 (m, 6H). | |
| 110 | 728.2 | ¹H NMR (500 MHz, DMSO-d₆) δ | 2 |
| | | 11.30 (s, 1H), 10.68 (s, 1H), 8.23 (d, | |
| | | J = 2.1 Hz, 1H), 7.91-7.88 (m, 1H), | |
| | | 7.87-7.82 (m, 2H), 7.53-7.40 (m, | |
| | | 3H), 7.32-7.24 (m, 2H), 1H), 6.76- | |
| | | 6.75 (m, 1H), 5.35-5.34 (m, 2H), | |
| | | 4.42-4.38 (m, 1H), 4.34-4.04 (m, | |
| | | 2H), 3.90-3.70 (m, 2H), 3.69 (s, | |
| | | 2H), 3.11-2.99 (m, 3H), 2.87-2.78 | |
| | | (m, 2H), 2.38-2.33 (m, 1H), 2.09- | |
| | | 1.90 (m, 6H). | |
| 111 | 748.2 | ¹H NMR (500 MHz, DMSO-d₆) δ | 2 |
| | | 8.23 (d, J = 2.1 Hz, 1H), 8.12 (d, J = | |
| | | 2.2 Hz, 1H), 7.91-7.88 (m, 1H), | |
| | | 7.69-7.67 (m, 1H), 7.60 (d, J = 7.9 | |
| | | Hz, 1H), 7.53-7.40 (m, 3H), 7.30- | |
| | | 7.17 (m, 3H), 6.76-6.75 (m, 1H), | |
| | | 5.35-5.34 (m, 2H), 4.35-4.04 (m, | |
| | | 3H), 3.90-3.67 (m, 2H), 3.69 (s, | |
| | | 2H), 3.11-2.78 (m, 5H), 2.38-2.33 | |
| | | (m, 1H), 2.09-1.92 (m, 6H). | |
| 112 | 720.3 | ¹H NMR (500 MHz, DMSO-d₆) δ | 2 |
| | | 12.64 (s, 1H), 10.83 (s, 1H), 8.23 (d, | |
| | | J = 2.1 Hz, 1H), 8.15-8.11 (m, 1H), | |
| | | 7.91-7.88 (m, 1H), 7.67 (dd, J = 7.8, | |
| | | 2.2 Hz, 1H), 7.58 (d, J = 7.9 Hz, 1H), | |
| | | 7.53-7.42 (m, 2H), 7.42 (d, J = 8.8 | |
| | | Hz, 1H), 7.30-7.17 (m, 3H), 6.76- | |
| | | 6.75 (m, 1H), 5.35-5.34 (m, 2H), 4.35- | |
| | | 4.26 (m, 2H), 4.04-3.67 (m, 3H), | |
| | | 3.69 (s, 2H), 3.11-2.78 (m, 5H), | |
| | | 2.38-2.33 (m, 2H), 2.09-1.93 (m, | |
| | | 5H), 0.99-0.82 (m, 4H). | |
| 113 | 788.3 | ¹H NMR (500 MHz, DMSO-d₆) δ | 2 |
| | | 12.71 (s, 1H), 11.23 (s, 1H), 8.23 (d, | |
| | | J = 2.1 Hz, 1H), 8.08-8.02 (m, 2H), | |
| | | 7.91-7.88 (m, 1H), 7.53-7.40 (m, | |
| | | 3H), 7.30-7.17 (m, 3H), 6.76-6.75 | |
| | | (m, 1H), 5.35-5.34 (m, 2H), 4.35- | |
| | | 4.04 (m, 2H), 3.90-3.71 (m, 2H), | |
| | | 3.69 (s, 2H), 3.11-2.78 (m, 5H), 2.36- | |
| | | 1.90 (m, 7H), 0.96-0.82 (m, 4H). | |
| 114 | 734.3 | ¹H NMR (500 MHz, DMSO-d₆) δ | 2 |
| | | 12.53 (s, 1H), 11.00 (s, 1H), 8.23 (d, | |
| | | J = 2.1 Hz, 1H), 7.90 (d, J = 2.2 Hz, | |
| | | 1H), 7.91-7.88 (m, 1H), 7.53-7.45 | |
| | | (m, 2H), 7.49-7.40 (m, 2H), 7.30- | |
| | | 7.17 (m, 3H), 6.76-6.75 (m, 1H), | |
| | | 5.35-5.34 (m, 2H), 4.42-4.38 (m, | |
| | | 1H), 4.32-4.28 (m, 1H), 4.06-4.01 | |
| | | (m, 1H), 3.90-3.82 (m, 1H), 3.75- | |
| | | 3.69 (m, 1H), 3.69 (s, 2H), 3.11-3.00 | |
| | | (m, 3H), 2.87-2.78 (m, 2H), 2.36- | |
| | | 1.90 (m, 7H), 0.96-0.81 (m, 4H). | |
| 115 | 730.2 | ¹H NMR (500 MHz, DMSO-d₆) δ | 2 |
| | | 8.23-8.18 (m, 2H), 7.91-7.88 (m, | |
| | | 1H), 7.68-7.66 (m, 1H), 7.62-7.59 | |
| | | (m, 1H), 7.52-7.40 (m, 3H), 7.29- | |
| | | 7.26 (m, 2H), 1H), 7.19-7.15 (m, | |
| | | 1H), 7.01 (d, J = 57.1 Hz, 1H), 6.76- | |
| | | 6.75 (m, 1H), 5.35-5.34 (m, 2H), 4.46- | |
| | | 4.21 (m, 2H), 4.04-3.69 (m, 3H), | |
| | | 3.69 (s, 2H), 3.15-2.76 (m, 5H), | |
| | | 2.42-2.22 (m, 1H), 2.14-1.88 (m, | |
| | | 5H). | |
| 116 | 744.3 | ¹H NMR (500 MHz, DMSO-d₆) δ | 2 |
| | | 11.97 (s, 1H), 11.05 (s, 1H), 8.23 (d, | |
| | | J = 2.1 Hz, 1H), 8.06 (d, J = 2.2 Hz, | |
| | | 1H), 7.91-7.88 (m, 1H), 7.54-7.37 | |
| | | (m, 4H), 7.32-7.31 (m, 1H), 7.25- | |
| | | 7.14 (m, 2H), 7.05-6.83 (m, 2H), | |
| | | 5.35-5.34 (m, 2H), 4.41-4.26 (m, | |
| | | 2H), 4.04-3.82 (m, 2H), 3.75-3.69 | |
| | | (m, 1H), 3.69 (s, 2H), 3.16-2.77 (m, | |
| | | 5H), 2.41-2.26 (m, 1H), 2.09-1.87 | |
| | | (m, 5H). | |
| 117 | 722.3 | ¹H NMR (500 MHz, DMSO-d₆) δ | 2 |
| | | 8.23 (d, J = 2.1 Hz, 1H), 7.91-7.88 | |
| | | (m, 1H), 7.78 (d, J = 2.2 Hz, 1H), | |
| | | 7.53-7.44 (m, 4H), 7.32-7.24 (m, | |
| | | 2H), 1H), 6.76-6.75 (m, 1H), 5.35- | |
| | | 5.34 (m, 2H), 4.40-3.82 (m, 2H), | |
| | | 3.76-3.68 (m, 3H), 3.11-2.99 (m, | |
| | | 3H), 2.87-2.69 (m, 4H), 2.33-1.92 | |
| | | (m, 6H), 1.28 (t, J = 7.6 Hz, 3H). | |
| 118 | 734.3 | ¹H NMR (500 MHz, DMSO-d₆) δ | 2 |
| | | 12.43 (s, 1H), 10.90 (s, 1H), 8.23 (d, | |
| | | J = 2.1 Hz, 1H), 7.91-7.88 (m, 1H), | |
| | | 7.83 (d, J = 2.2 Hz, 1H), 7.57 (d, J = | |
| | | 2.2 Hz, 1H), 7.53-7.40 (m, 3H), | |
| | | 7.32-7.24 (m, 2H), 1H), 6.76-6.75 | |
| | | (m, 1H), 5.35-5.34 (m, 2H), 4.38- | |
| | | 4.27 (m, 2H), 4.04-3.68 (m, 5H), | |
| | | 3.51-3.48 (m, 1H), 3.11-2.78 (m, | |
| | | 5H), 2.38-2.33 (m, 1H), 2.09-1.90 | |
| | | (m, 5H), 1.13-0.99 (m, 4H). | |
| 119 | 744.3 | ¹H NMR (500 MHz, DMSO-d₆) δ | 2 |
| | | 12.95 (s, 1H), 10.87 (s, 1H), 8.23 (d, | |
| | | J = 2.1 Hz, 1H), 7.91-7.88 (m, 1H), | |
| | | 7.86 (d, J = 2.0 Hz, 1H), 7.62 (dd, J = | |
| | | 2.2, 0.7 Hz, 2H), 7.53-7.40 (m, 3H), | |
| | | 7.32-7.24 (m, 2H), 1H), 6.76-6.75 | |
| | | (m, 1H), 5.35-5.34 (m, 2H), 4.40- | |
| | | 4.26 (m, 2H), 4.04-3.73 (m, 3H), | |
| | | 3.69 (s, 2H), 3.11-2.99 (m, 3H), | |
| | | 2.87-2.78 (m, 2H), 2.33-1.92 (m, | |
| | | 6H). | |
| 120 | 722.3 | ¹H NMR (500 MHz, DMSO-d₆) δ | 2 |
| | | 8.23 (d, J = 2.1 Hz, 1H), 7.91-7.88 | |
| | | (m, 1H), 7.50 (t, J = 7.9 Hz, 1H), | |
| | | 7.49-7.45 (m, 1H), 7.46-7.39 (m, | |
| | | 2H), 7.32-7.24 (m, 2H), 1H), 6.76- | |
| | | 6.75 (m, 1H), 5.35-5.34 (m, 2H), | |
| | | 4.42-4.38 (m, 1H), 4.32-4.28 (m, | |
| | | 1H), 4.04-3.67 (m, 3H), 3.69 (s, | |
| | | 2H), 3.11-2.78 (m, 5H), 2.73 (s, | |
| | | 3H), 2.36-1.90 (m, 6H). | |
| 121 | 726.6 | ¹H NMR (500 MHz, DMSO-d₆) δ | 2 |
| | | 8.23 (d, J = 2.1 Hz, 1H), 7.91-7.88 | |
| | | (m, 1H), 7.61 (d, J = 4.9 Hz, 1H), | |
| | | 7.53-7.40 (m, 3H), 7.32-7.24 (m, | |
| | | 2H), 1H), 6.76-6.75 (m, 1H), 5.35- | |
| | | 5.34 (m, 2H), 4.39-4.24 (m, 2H), | |
| | | 4.04-3.82 (m, 2H), 3.77-3.69 (m, | |
| | | 1H), 3.69 (s, 2H), 3.11-2.78 (m, | |
| | | 5H), 2.74 (s, 3H), 2.38-2.33 (m, | |
| | | 1H), 2.09-1.90 (m, 5H). | |
| 122 | 712.1 | ¹H NMR (500 MHz, DMSO-d₆) δ | 2 |
| | | 8.23 (d, J = 2.1 Hz, 1H), 7.91-7.88 | |
| | | (m, 1H), 7.77-7.75 (m, 1H), 7.53- | |
| | | 7.51 (m, 1H), 7.46-7.44 (m, 1H), | |
| | | 7.43 (dd, J = 8.9, 5.2 Hz, 2H), 7.32- | |
| | | 7.24 (m, 2H), 1H), 6.76-6.75 (m, | |
| | | 1H), 5.35-5.34 (m, 2H), 4.39-4.25 | |
| | | (m, 2H), 4.04-3.68 (m, 5H), 3.11- | |
| | | 2.99 (m, 3H), 2.87-2.78 (m, 2H), | |
| | | 2.36-1.90 (m, 6H). | |
| 123 | 712.1 | ¹H NMR (500 MHz, DMSO-d₆) δ | 2 |
| | | 8.36 (d, J = 2.1 Hz, 1H), 8.01 (d, J = | |
| | | 5.1 Hz, 1H), 7.91-7.88 (m, 1H), 7.53- | |
| | | 7.40 (m, 3H), 7.36-7.28 (m, 2H), | |
| | | 7.25-7.19 (m, 2H), 6.76-6.75 (m, | |
| | | 1H), 5.35-5.34 (m, 2H), 4.39-4.23 | |
| | | (m, 2H), 4.04-3.69 (m, 3H), 3.69 (s, | |
| | | 2H), 3.11-2.78 (m, 7H), 2.36-1.90 | |
| | | (m, 6H). | |
| 124 | 725.3 | ¹H NMR (500 MHz, DMSO-d₆) δ | 2 |
| | | 9.98 (s, 1H), 9.87 (s, 1H), 8.23 (d, J = | |
| | | 2.2 Hz, 1H), 7.96-7.92 (m, 1H), 7.65- | |
| | | 7.46 (m, 4H), 7.42 (d, J = 8.8 Hz, | |
| | | 1H), 7.11-6.97 (m, 2H), 5.35-5.34 | |
| | | (m, 2H), 4.42-4.27 (m, 2H), 4.04- | |
| | | 3.70 (m, 3H), 3.69 (s, 2H), 3.15- | |
| | | 2.82 (m, 5H), 2.32-2.22 (m, 2H), | |
| | | 2.22 (s, 3H), 2.18-2.09 (m, 2H), | |
| | | 2.08-1.58 (m, 6H). | |
| 125 | 730.3 | ¹H NMR (500 MHz, DMSO-d₆) δ | 2 |
| | | 10.17 (s, 1H), 9.97 (s, 1H), 8.23 (d, J = | |
| | | 2.2 Hz, 1H), 7.96-7.92 (m, 1H), | |
| | | 7.63-7.44 (m, 6H), 7.42 (d, J = 8.8 | |
| | | Hz, 1H), 7.11-6.97 (m, 2H), 5.35- | |
| | | 5.34 (m, 2H), 4.43-4.20 (m, 2H), | |
| | | 4.04-3.70 (m, 3H), 3.69 (s, 2H), | |
| | | 3.11-2.78 (m, 5H), 2.23-1.90 (m, | |
| | | 8H), 1.75-1.58 (m, 2H). | |
| 126 | 703.3 | ¹H NMR (500 MHz, DMSO-d₆) δ | 2 |
| | | 12.44 (s, 1H), 10.82 (s, 1H), 8.23 (d, | |
| | | J = 2.1 Hz, 1H), 7.91-7.88 (m, 1H), | |
| | | 7.82 (d, J = 2.2 Hz, 1H), 7.63-7.61 | |
| | | (m, 1H), 7.55-7.47 (m, 4H), 7.42 (d, | |
| | | J = 8.8 Hz, 1H), 7.11-6.97 (m, 2H), | |
| | | 5.35-5.34 (m, 2H), 4.33-4.28 (m, | |
| | | 2H), 4.04-3.68 (m, 6H), 3.11-2.78 | |
| | | (m, 5H), 2.36-1.90 (m, 6H). | |
| 127 | 699.3 | ¹H NMR (500 MHz, DMSO-d₆) δ | 2 |
| | | 12.00 (s, 1H), 10.69 (s, 1H), 8.23 (d, | |
| | | J = 2.1 Hz, 1H), 7.91-7.88 (m, 1H), | |
| | | 7.70 (d, J = 2.2 Hz, 1H), 7.63-7.61 | |
| | | (m, 1H), 7.56-7.52 (m, 1H), 7.50- | |
| | | 7.45 (m, 3H), 7.42 (d, J = 8.8 Hz, | |
| | | 1H), 7.11-6.97 (m, 2H), 5.35-5.34 | |
| | | (m, 2H), 4.33-4.28 (m, 2H), 4.04- | |
| | | 3.68 (m, 6H), 3.11-2.99 (m, 3H), | |
| | | 2.87-2.78 (m, 2H), 2.73 (s, 3H), | |
| | | 2.46 (s, 3H), 2.33-1.92 (m, 6H). | |
| 128 | 712.3 | ¹H NMR (500 MHz, DMSO-d₆) δ | 2 |
| | | 9.98 (s, 1H), 9.90 (s, 1H), 8.23 (d, J = | |
| | | 2.2 Hz, 1H), 7.96-7.92 (m, 1H), 7.75 | |
| | | (d, J = 2.1 Hz, 1H), 7.61-7.44 (m, | |
| | | 5H), 7.42 (d, J = 8.8 Hz, 1H), 7.33 (d, | |
| | | J = 7.9 Hz, 1H), 7.11-6.97 (m, 2H), | |
| | | 5.35-5.34 (m, 2H), 4.35-4.26 (m, | |
| | | 2H), 4.04-3.70 (m, 4H), 3.69 (s, | |
| | | 2H), 3.11-2.78 (m, 5H), 2.30-1.90 | |
| | | (m, 8H), 1.73-1.61 (m, 2H). | |
| 129 | 689.3 | ¹H NMR (500 MHz, DMSO-d₆) δ | 2 |
| | | 8.19 (dd, J = 11.9, 2.0 Hz, 2H), 7.94- | |
| | | 7.86 (m, 2H), 7.65 (dd, J = 7.9, 2.1 | |
| | | Hz, 1H), 7.63-7.61 (m, 1H), 7.56- | |
| | | 7.44 (m, 3H), 7.42 (d, J = 8.8 Hz, | |
| | | 1H), 7.11-6.97 (m, 2H), 5.35-5.34 | |
| | | (m, 2H), 4.40-4.25 (m, 2H), 4.06- | |
| | | 4.01 (m, 1H), 3.87-3.79 (m, 1H), | |
| | | 3.78-3.74 (m, 1H), 3.69 (s, 2H), | |
| | | 3.11-2.78 (m, 5H), 2.30-1.90 (m, | |
| | | 6H). | |
| 130 | 739.3 | ¹H NMR (500 MHz, DMSO-d₆) δ | 2 |
| | | 13.03 (s, 1H), 10.94 (s, 1H), 8.23 (d, | |
| | | J = 2.1 Hz, 1H), 8.12 (d, J = 2.2 Hz, | |
| | | 1H), 7.91-7.88 (m, 1H), 7.69-7.67 | |
| | | (m, 1H), 7.63-7.59 (m, 2H), 7.56- | |
| | | 7.47 (m, 3H), 7.42 (d, J = 8.8 Hz, | |
| | | 1H), 7.11-6.97 (m, 2H), 5.35-5.34 | |
| | | (m, 2H), 4.38-4.26 (m, 2H), 4.04- | |
| | | 3.82 (m, 2H), 3.76-3.68 (m, 3H), | |
| | | 3.11-2.78 (m, 5H), 2.31-1.90 (m, | |
| | | 6H). | |
| 131 | 718.3 | ¹H NMR (500 MHz, DMSO-d₆) δ | 2 |
| | | 9.68 (s, 1H), 9.57 (s, 1H), 8.23 (d, J = | |
| | | 2.2 Hz, 1H), 7.96-7.92 (m, 1H), 7.65- | |
| | | 7.46 (m, 4H), 7.42 (d, J = 8.8 Hz, | |
| | | 1H), 7.05-6.97 (m, 2H), 5.35-5.34 | |
| | | (m, 2H), 4.42-4.27 (m, 2H), 4.04- | |
| | | 3.70 (m, 3H), 3.69 (s, 2H), 3.15- | |
| | | 2.82 (m, 5H), 2.32-2.22 (m, 2H), | |
| | | 2.22 (s, 3H), 2.18-2.09 (m, 2H), | |
| | | 2.08-1.58 (m, 6H). | |
| 132 | 735.3 | ¹H NMR (500 MHz, DMSO-d₆) δ | 2 |
| | | 12.97 (s, 1H), 11.15 (s, 1H), 8.23 (d, | |
| | | J = 2.1 Hz, 1H), 8.06 (d, J = 2.2 Hz, | |
| | | 1H), 7.91-7.88 (m, 1H), 7.63-7.61 | |
| | | (m, 1H), 7.56-7.40 (m, 5H), 7.21- | |
| | | 7.14 (m, 1H), 6.76-6.75 (m, 1H), | |
| | | 5.35-5.34 (m, 2H), 4.33-4.28 (m, | |
| | | 2H), 4.04-3.68 (m, 6H), 3.11-3.00 | |
| | | (m, 3H), 2.87-2.78 (m, 2H), 2.38- | |
| | | 2.33 (m, 1H), 2.09-1.90 (m, 5H). | |
| 133 | 685.3 | ¹H NMR (500 MHz, DMSO-d₆) δ | 2 |
| | | 8.23 (d, J = 2.1 Hz, 1H), 7.92-7.89 | |
| | | (m, 2H), 7.69-7.67 (m, 1H), 7.63- | |
| | | 7.61 (m, 1H), 7.56-7.44 (m, 4H), | |
| | | 7.42 (d, J = 8.8 Hz, 1H), 7.11-6.97 | |
| | | (m, 2H), 5.35-5.34 (m, 2H), 4.33- | |
| | | 4.28 (m, 2H), 4.04-3.68 (m, 5H), | |
| | | 3.11-2.78 (m, 8H), 2.38-2.33 (m, | |
| | | 1H), 2.09-1.90 (m, 5H). | |
| 134 | 685.3 | ¹H NMR (500 MHz, DMSO-d₆) δ | 2 |
| | | 12.710 (s, 1H), 10.15 (s, 1H), 8.22 | |
| | | (dd, J = 14.1, 1.8 Hz, 2H), 7.91-7.88 | |
| | | (m, 1H), 7.84 (t, J = 2.0 Hz, 1H), | |
| | | 7.63-7.61 (m, 1H), 7.56-7.50 (m, | |
| | | 2H), 7.50-7.45 (m, 2H), 7.42 (d, J = | |
| | | 8.8 Hz, 1H), 7.11-6.97 (m, 2H), | |
| | | 5.35-5.34 (m, 2H), 4.33-4.28 (m, | |
| | | 2H), 4.04-3.78 (m, 5H), 3.10-2.78 | |
| | | (m, 5H), 2.33-1.90 (m, 6H). | |
| 135 | 699.3 | ¹H NMR (500 MHz, DMSO-d₆) δ | 2 |
| | | 12.53 (s, 1H), 10.67 (s, 1H), 8.23 (d, | |
| | | J = 2.1 Hz, 1H), 7.93-7.86 (m, 2H), | |
| | | 7.67 (dd, J = 7.8, 2.2 Hz, 1H), 7.63- | |
| | | 7.61 (m, 1H), 7.56-7.44 (m, 4H), | |
| | | 7.42 (d, J = 8.8 Hz, 1H), 7.11-6.97 | |
| | | (m, 2H), 5.35-5.34 (m, 2H), 4.33- | |
| | | 4.28 (m, 2H), 4.04-3.68 (m, 5H), | |
| | | 3.11-2.99 (m, 3H), 2.87-2.76 (m, | |
| | | 4H), 2.38-2.33 (m, 1H), 2.09-1.90 | |
| | | (m, 5H), 1.33 (t, J = 7.6 Hz, 3H). | |
| 136 | 693.2 | ¹H NMR (500 MHz, DMSO-d₆) δ | 3 |
| | | 8.07-8.00 (m, 2H), 7.88 (d, J = 1.6 | |
| | | Hz, 1H), 7.65-7.64 (m, 1H), 7.53- | |
| | | 7.49 (m, 3H), 7.43-7.32 (m, 1H), | |
| | | 7.29-7.26 (m, 2H), 1H), 6.83-6.81 | |
| | | (m, 1H), 6.47-6.45 (m, 1H), 5.34- | |
| | | 5.31 (m, 2H), 4.33-4.29 (m, 2H), | |
| | | 4.25-4.21 (m, 1H), 4.03-3.70 (m, | |
| | | 5H), 3.33-3.29 (m, 2H), 3.04-2.97 | |
| | | (m, 2H), 2.91-2.82 (m, 5H), 2.19- | |
| | | 2.10 (m, 2H). | |
| 137 | 693.2 | ¹H NMR (500 MHz, DMSO-d₆) δ | 3 |
| | | 8.17 (d, J = 1.7 Hz, 1H), 8.08-7.99 | |
| | | (m, 2H), 7.84 (t, J = 2.4 Hz, 1H), | |
| | | 7.56-7.39 (m, 4H), 7.29-7.26 (m, | |
| | | 2H), 1H), 6.83-6.81 (m, 1H), 6.50- | |
| | | 6.40 (m, 1H), 5.34-5.31 (m, 2H), 4.37- | |
| | | 4.28 (m, 2H), 4.26-4.17 (m, 1H), | |
| | | 4.03-3.70 (m, 5H), 3.35-3.26 (m, | |
| | | 2H), 3.06-2.95 (m, 2H), 2.92-2.82 | |
| | | (m, 2H), 2.34-2.15 (m, 2H). | |
| 138 | 708.2 | ¹H NMR (500 MHz, DMSO-d₆) δ | 3 |
| | | 12.32 (s, 1H), 10.54 (s, 1H), 8.23 (d, | |
| | | J = 2.2 Hz, 1H), 8.12 (s, 1H), 7.96- | |
| | | 7.92 (m, 1H), 7.70-7.62 (m, 2H), | |
| | | 7.52-7.49 (m, 1H), 7.47-7.43 (m, | |
| | | 2H), 7.42 (d, J = 1.8 Hz, 1H), 7.29- | |
| | | 7.26 (m, 2H), 1H), 6.83-6.81 (m, | |
| | | 1H), 6.47-6.45 (m, 1H), 5.34-5.31 | |
| | | (m, 2H), 4.41-4.23 (m, 2H), 4.13 (s, | |
| | | 3H), 4.04-3.70 (m, 5H), 3.34-3.27 | |
| | | (m, 2H), 3.04-2.97 (m, 2H), 2.91- | |
| | | 2.82 (m, 2H), 2.19-2.10 (m, 2H). | |
| 139 | 696.2 | ¹H NMR (500 MHz, DMSO-d₆) δ | 3 |
| | | 12.92 (s, 1H), 10.71 (s, 1H), 8.23 (d, | |
| | | J = 2.2 Hz, 1H), 8.18-8.14 (m, 1H), | |
| | | 7.96-7.92 (m, 1H), 7.69-7.62 (m, | |
| | | 2H), 7.52-7.49 (m, 1H), 7.47-7.43 | |
| | | (m, 2H), 7.42 (d, J = 1.8 Hz, 1H), | |
| | | 7.29-7.26 (m, 2H), 1H), 6.83-6.81 | |
| | | (m, 1H), 6.47-6.45 (m, 1H), 5.34- | |
| | | 5.31 (m, 2H), 4.41-4.25 (m, 2H), | |
| | | 4.04-3.70 (m, 5H), 3.31-2.27 (m, | |
| | | 2H), 3.04-2.97 (m, 2H), 2.91-2.82 | |
| | | (m, 2H), 2.19-2.10 (m, 2H). | |
| 140 | 720.3 | ¹H NMR (500 MHz, DMSO-d₆) δ | 3 |
| | | 12.52 (s, 1H), 10.83 (s, 1H), 8.23 (d, | |
| | | J = 2.2 Hz, 1H), 8.03 (d, J = 2.1 Hz, | |
| | | 1H), 7.96-7.92 (m, 1H), 7.67 (d, J = | |
| | | 7.8 Hz, 1H), 7.58 (d, J = 7.9 Hz, 1H), | |
| | | 7.52-7.49 (m, 1H), 7.47-7.43 (m, | |
| | | 2H), 7.42 (d, J = 1.8 Hz, 1H), 7.29- | |
| | | 7.26 (m, 2H), 1H), 6.83-6.81 (m, | |
| | | 1H), 6.47-6.45 (m, 1H), 5.34-5.31 | |
| | | (m, 2H), 4.42-4.23 (m, 2H), 4.04- | |
| | | 3.70 (m, 5H), 3.34-3.37 (m, 2H), | |
| | | 3.17-3.11 (m, 1H), 3.04-2.97 (m, | |
| | | 2H), 2.91-2.82 (m, 2H), 2.19-2.10 | |
| | | (m, 2H), 1.65 (d, J = 6.6 Hz, 3H), | |
| | | 1.60 (d, J = 6.6 Hz, 3H). | |
| 141 | 678.2 | ¹H NMR (500 MHz, DMSO-d₆) δ | 3 |
| | | 12.31 (s, 1H), 10.09 (s, 1H), 8.23 (d, | |
| | | J = 1.8 Hz, 2H), 8.04 (t, J = 2.1 Hz, | |
| | | 1H), 7.91-7.88 (m, 1H), 7.69-7.67 | |
| | | (m, 1H), 7.55-7.49 (m, 2H), 7.49- | |
| | | 7.43 (m, 2H), 7.42 (d, J = 1.8 Hz, | |
| | | 1H), 7.29-7.26 (m, 2H), 1H), 6.83- | |
| | | 6.81 (m, 1H), 6.47-6.45 (m, 1H), | |
| | | 5.34-5.31 (m, 2H), 4.44-4.23 (m, | |
| | | 2H), 4.04-3.67 (m, 5H), 3.34-3.27 | |
| | | (m, 2H), 3.04-2.82 (m, 4H), 2.38- | |
| | | 2.33 (m, 1H), 2.09-2.02 (m, 1H). | |
| 142 | 694.2 | ¹H NMR (500 MHz, DMSO-d₆) δ | 3 |
| | | 8.37 (s, 1H), 7.97 (d, J = 2.2 Hz, 1H), | |
| | | 7.87 (dd, J = 8.5, 2.0 Hz, 1H), 7.56 | |
| | | (dd, J = 8.8, 2.2 Hz, 1H), 7.53-7.40 | |
| | | (m, 5H), 7.29-7.26 (m, 2H), 1H), | |
| | | 6.83-6.81 (m, 1H), 6.47-6.45 (m, | |
| | | 1H), 5.34-5.31 (m, 2H), 4.42-4.24 | |
| | | (m, 2H), 4.02-3.70 (m, 5H), 3.34- | |
| | | 3.27 (m, 2H), 3.04-2.82 (m, 4H), | |
| | | 2.19-2.10 (m, 2H). | |
| 143 | 719.3 | ¹H NMR (500 MHz, DMSO-d₆) δ | 3 |
| | | 9.86 (s, 1H), 9.76 (s, 1H), 8.23 (d, J = | |
| | | 2.2 Hz, 1H), 7.96-7.92 (m, 1H), 7.75 | |
| | | (d, J = 2.1 Hz, 1H), 7.62 (dd, J = 7.8, | |
| | | 2.0 Hz, 1H), 7.52-7.49 (m, 1H), | |
| | | 7.47-7.43 (m, 2H), 7.42 (d, J = | |
| | | 1.8 Hz, 1H), 7.35-7.28 (m, 2H), 7.21 | |
| | | (dd, J = 7.8, 2.2 Hz, 1H), 6.83-6.81 | |
| | | (m, 1H), 6.47-6.45 (m, 1H), 5.34- | |
| | | 5.31 (m, 2H), 4.39-4.32 (m, 1H), | |
| | | 4.32-4.26 (m, 1H), 4.04-3.70 (m, | |
| | | 5H), 3.34-3.27 (m, 2H), 3.04-2.82 | |
| | | (m, 4H), 2.30-2.19 (m, 2H), 2.17- | |
| | | 2.09 (m, 2H), 1.73-1.61 (m, 2H). | |
| 144 | 680.2 | H NMR (500 MHz, DMSO-d₆) δ | 3 |
| | | 8.66 (s, 1H), 8.40 (d, J = 8.1 Hz, 1H), | |
| | | 8.19 (d, J = 2.1 Hz, 1H), 7.91 (dd, J = | |
| | | 8.8, 2.0 Hz, 1H), 7.53-7.52 (m, 1H), | |
| | | 7.45-7.40 (m, 3H), 7.29-7.26 (m, | |
| | | 2H), 1H), 6.83-6.81 (m, 1H), 6.47- | |
| | | 6.45 (m, 1H), 5.34-5.31 (m, 2H), 4.38- | |
| | | 4.25 (m, 2H), 4.04-3.67 (m, 5H), | |
| | | 3.34-3.27 (m, 2H), 3.04-2.82 (m, | |
| | | 4H), 2.38-2.33 (m, 1H), 2.09- | |
| | | 2.02 (m, 1H). | |
| 145 | 675.3 | ¹H NMR (500 MHz, Chloroform-d) δ | 3 |
| | | 8.43 (d, J = 2.0 Hz, 1H), 8.15 (d, J = | |
| | | 1.9 Hz, 1H), 7.85-7.84 (m, 2H), 7.63- | |
| | | 7.58 (m, 2H), 7.49-7.43 (m, 2H), | |
| | | 7.43-7.37 (m, 12H), 6.80-6.65 (m, | |
| | | 2H), 5.22 (s, 2H), 4.31-4.28 (m, 2H), | |
| | | 4.17-4.10 (m, 1H), 4.02-3.95 (m, | |
| | | 2H), 3.83-3.74 (m, 2H), 3.35-3.31 | |
| | | (m, 2H), 3.08-2.86 (m, 7H), 2.31- | |
| | | 2.17 (m, 2H). | |
| 146 | 708.2 | ¹H NMR (500 MHz, DMSO-d₆) δ | 3 |
| | | 10.74 (s, 1H), 9.91 (s, 1H), 8.23 (d, J = | |
| | | 2.1 Hz, 1H), 7.91-7.88 (m, 1H), | |
| | | 7.57-7.40 (m, 6H), 7.34-7.28 (m, | |
| | | 2H), 7.21 (dd, J = 7.8, 2.2 Hz, 1H), | |
| | | 6.83-6.81 (m, 1H), 6.47-6.45 (m, | |
| | | 1H), 5.34-5.31 (m, 2H), 4.35-4.26 | |
| | | (m, 2H), 4.08-3.82 (m, 4H), 3.75- | |
| | | 3.69 (m, 1H), 3.34-3.27 (m, 2H), | |
| | | 3.04-2.82 (m, 4H), 2.38-2.33 (m, | |
| | | 1H), 2.09-2.02 (m, 1H). | |
| 147 | 694.2 | ¹H NMR (500 MHz, DMSO-d₆) δ | 3 |
| | | 10.54 (s, 2H), 10.01 (s, 1H), 8.31 (d, | |
| | | J = 2.1 Hz, 1H), 8.23 (d, J = 2.1 Hz, | |
| | | 1H), 7.92-7.84 (m, 2H), 7.53-7.44 | |
| | | (m, 3H), 7.42 (dd, J = 8.2, 1.3 Hz, | |
| | | 2H), 7.29-7.26 (m, 2H), 1H), 6.83- | |
| | | 6.81 (m, 1H), 6.47-6.45 (m, 1H), | |
| | | 5.34-5.31 (m, 2H), 4.43-4.24 (m, | |
| | | 2H), 4.04-3.67 (m, 5H), 3.34-3.27 | |
| | | (m, 2H), 3.04-2.82 (m, 4H), 2.38- | |
| | | 2.33 (m, 1H), 2.09-2.02 (m, 1H). | |
| 148 | 678.2 | ¹H NMR (500 MHz, DMSO-d₆) δ | 3 |
| | | 10.24 (s, 1H), 8.44 (d, J = 7.1 Hz, | |
| | | 1H), 8.25 (d, J = 3.6 Hz, 1H), 8.23 (d, | |
| | | J = 1.7 Hz, 1H), 8.04 (d, J = 1.8 Hz, | |
| | | 1H), 7.91-7.88 (m, 1H), 7.53-7.40 | |
| | | (m, 5H), 7.29-7.26 (m, 2H), 1H), | |
| | | 6.83-6.81 (m, 1H), 6.82 (d, J = 3.7 | |
| | | Hz, 1H), 6.47-6.45 (m, 1H), 5.34- | |
| | | 5.31 (m, 2H), 4.41-4.37 (m, 1H), 4.25- | |
| | | 4.18 (m, 1H), 4.08-3.68 (m, 5H), | |
| | | 3.34-3.27 (m, 2H), 3.04-2.82 (m, | |
| | | 4H), 2.32-2.14 (m, 2H). | |
| 149 | 712.2 | ¹H NMR (500 MHz, DMSO-d₆) δ | 3 |
| | | 10.29 (s, 1H), 8.51-8.46 (m, 1H), | |
| | | 8.32 (dd, J = 12.7, 1.6 Hz, 2H), 8.04 | |
| | | (s, 1H), 7.91-7.88 (m, 1H), 7.62-7.60 | |
| | | (m, 1H), 7.52-7.49 (m, 1H), 7.47- | |
| | | 7.43 (m, 2H), 7.42 (d, J = 1.8 Hz, | |
| | | 1H), 7.29-7.26 (m, 2H), 1H), 6.83- | |
| | | 6.81 (m, 1H), 6.47-6.45 (m, 1H), | |
| | | 5.34-5.31 (m, 2H), 4.41-4.37 (m, | |
| | | 1H), 4.25-4.18 (m, 1H), 4.04-3.68 | |
| | | (m, 5H), 3.34-3.27 (m, 2H), 3.04- | |
| | | 2.97 (m, 2H), 2.91-2.82 (m, 2H), | |
| | | 2.32-2.14 (m, 2H). | |
| 150 | 695.2 | ¹H NMR (500 MHz, DMSO-d₆) δ | 3 |
| | | 9.81 (s, 1H), 9.18 (s, 1H), 8.37 (d, J = | |
| | | 2.2 Hz, 1H), 8.23 (d, J = 2.1 Hz, 1H), | |
| | | 7.91-7.88 (m, 1H), 7.81-7.76 (m, | |
| | | 1H), 7.71 (dd, J = 7.8, 2.2 Hz, 1H), | |
| | | 7.52-7.49 (m, 1H), 7.47-7.43 (m, | |
| | | 2H), 7.42 (d, J = 1.8 Hz, 1H), 7.29- | |
| | | 7.26 (m, 2H), 1H), 6.83-6.81 (m, | |
| | | 1H), 6.47-6.45 (m, 1H), 5.34-5.31 | |
| | | (m, 2H), 4.37-4.33 (m, 1H), 4.31- | |
| | | 4.26 (m, 1H), 4.04-3.70 (m, 5H), | |
| | | 3.34-3.27 (m, 2H), 3.04-2.97 (m, | |
| | | 2H), 2.91-2.82 (m, 2H), 2.19-2.10 | |
| | | (m, 2H). | |
| 151 | 706.3 | ¹H NMR (500 MHz, DMSO-d₆) δ | 3 |
| | | 12.00 (s, 1H), 10.69 (s, 1H), 8.23 (d, | |
| | | J = 2.1 Hz, 1H), 7.91-7.88 (m, 1H), | |
| | | 7.70 (d, J = 2.2 Hz, 1H), 7.53-7.40 | |
| | | (m, 5H), 7.29-7.26 (m, 2H), 1H), | |
| | | 6.83-6.81 (m, 1H), 6.47-6.45 (m, | |
| | | 1H), 5.34-5.31 (m, 2H), 4.42-4.38 | |
| | | (m, 1H), 4.32-4.28 (m, 1H), 4.04- | |
| | | 3.67 (m, 5H), 3.34-3.27 (m, 2H), | |
| | | 3.04-2.82 (m, 4H), 2.46 (s, 3H), | |
| | | 2.38-2.33 (m, 1H), 2.09-2.02 (m, | |
| | | 1H). | |
| 152 | 696.2 | ¹H NMR (500 MHz, DMSO-d₆) δ | 3 |
| | | 8.21 (dd, J = 11.9, 2.0 Hz, 2H), 7.93- | |
| | | 7.90 (m, 2H), 7.65 (dd, J = 7.9, 2.1 | |
| | | Hz, 1H), 7.52-7.49 (m, 1H), 7.47- | |
| | | 7.43 (m, 2H), 7.42 (d, J = 1.8 Hz, | |
| | | 1H), 7.29-7.26 (m, 2H), 1H), 6.83- | |
| | | 6.81 (m, 1H), 6.47-6.45 (m, 1H), | |
| | | 5.34-5.31 (m, 2H), 4.43-4.21 (m, | |
| | | 2H), 4.04-3.70 (m, 5H), 3.34-3.27 | |
| | | (m, 2H), 3.04-2.82 (m, 4H), 2.19- | |
| | | 2.10 (m, 2H). | |
| 153 | 710.2 | ¹H NMR (500 MHz, DMSO-d₆) δ | 3 |
| | | 8.23 (d, J = 2.1 Hz, 1H), 7.91-7.88 | |
| | | (m, 1H), 7.82 (d, J = 2.2 Hz, 1H), | |
| | | 7.55 (dd, J = 8.1, 2.2 Hz, 1H), 7.52- | |
| | | 7.45 (m, 2H), 7.45-7.40 (m, 3H), | |
| | | 7.29-7.26 (m, 2H), 1H), 6.83-6.81, | |
| | | (m, 1H), 6.47-6.45 (m, 1H), 5.34- | |
| | | 5.31 (m, 2H), 4.43-4.21 (m, 2H), | |
| | | 4.08-3.67 (m, 5H), 3.34-3.27 (m, | |
| | | 2H), 3.04-2.82 (m, 4H), 2.43 (s, | |
| | | 3H), 2.38-2.33 (m, 1H), 2.05-2.00 | |
| | | (m, 1H). | |
| 154 | 737.2 | ¹H NMR (500 MHz, DMSO-d₆) δ | 3 |
| | | 10.17 (s, 1H), 9.97 (s, 1H), 8.23 (d, J = | |
| | | 2.2 Hz, 1H), 7.96-7.92 (m, 1H), | |
| | | 7.63-7.56 (m, 2H), 7.52-7.49 (m, | |
| | | 1H), 7.47-7.43 (m, 2H), 7.42 (d, J = | |
| | | 1.8 Hz, 1H), 7.29-7.26 (m, 2H), | |
| | | 1H), 6.83-6.81 (m, 1H), 6.47-6.45 | |
| | | (m, 1H), 5.34-5.31 (m, 2H), 4.38- | |
| | | 4.27 (m, 2H), 4.04-3.70 (m, 5H), | |
| | | 3.34-3.27 (m, 2H), 3.04-2.82 (m, | |
| | | 4H), 2.30-2.09 (m, 4H), 1.73-1.65 | |
| | | (m, 2H). | |
| 155 | 733.3 | ¹H NMR (500 MHz, DMSO-d₆) δ | 3 |
| | | 10.31 (s, 1H), 10.05 (s, 1H), 8.23 (d, | |
| | | J = 2.2 Hz, 1H), 7.96-7.92 (m, 1H), | |
| | | 7.65 (d, J = 2.2 Hz, 1H), 7.57-7.56 | |
| | | (m, 1H), 7.52-7.49 (m, 1H), 7.47- | |
| | | 7.43 (m, 2H), 7.42 (d, J = 1.8 Hz, | |
| | | 1H), 7.29-7.26 (m, 2H), 1H), 6.83- | |
| | | 6.81 (m, 1H), 6.47-6.45 (m, 1H), | |
| | | 5.34-5.31 (m, 2H), 4.33-4.28 (m, | |
| | | 2H), 4.04-3.70 (m, 5H), 3.34-3.27 | |
| | | (m, 2H), 3.04-2.82 (m, 4H), 2.43 (s, | |
| | | 3H), 2.30-2.20 (m, 5H), 2.17-1.71 | |
| | | (m, 4H). | |
| 156 | 760.2 | ¹H NMR (500 MHz, DMSO-d₆) δ | 3 |
| | | 12.71 (s, 1H), 10.84 (s, 1H), 8.23 (d, | |
| | | J = 2.1 Hz, 1H), 8.08 (d, J = 2.2 Hz, | |
| | | 1H), 7.91-7.88 (m, 1H), 7.86 (d, J = | |
| | | 2.2 Hz, 1H), 7.52-7.44 (m, 2H), | |
| | | 7.42 (dd, J = 8.2, 1.3 Hz, 2H), 7.29- | |
| | | 7.26 (m, 2H), 1H), 6.83-6.81 (m, | |
| | | 1H), 6.47-6.45 (m, 1H), 5.34-5.31 | |
| | | (m, 2H), 4.33-4.28 (m, 2H), 4.08- | |
| | | 3.67 (m, 5H), 3.34-3.27 (m, 2H), | |
| | | 3.04-2.82 (m, 4H), 2.43 (s, 3H), 2.13- | |
| | | 1.92 (m, 2H). | |
| 157 | 787.2 | ¹H NMR (500 MHz, DMSO-d₆) δ | 3 |
| | | 10.02 (s, 1H), 9.94 (s, 1H), 8.23 (d, J = | |
| | | 2.2 Hz, 1H), 8.11 (d, J = 2.2 Hz, | |
| | | 1H), 7.96-7.92 (m, 1H), 7.62 (d, J = | |
| | | 2.2 Hz, 1H), 7.52-7.49 (m, 1H), | |
| | | 7.47-7.43 (m, 2H), 7.42 (d, J = | |
| | | 1.8 Hz, 1H), 7.29-7.26 (m, 2H), | |
| | | 1H), 6.83-6.81 (m, 1H), 6.47-6.45 | |
| | | (m, 1H), 5.34-5.31 (m, 2H), 4.40- | |
| | | 4.25 (m, 2H), 4.04-3.70 (m, 5H), | |
| | | 3.34-3.27 (m, 2H), 3.04-2.28 (m, | |
| | | 4H), 2.30-2.26 (m, 4H), 1.72-1.59 | |
| | | (m, 2H). | |
| 158 | 746.2 | ¹H NMR (500 MHz, DMSO-d₆) δ | 3 |
| | | 8.19 (d, J = 12.0 Hz, 2H), 8.04 (d, J = | |
| | | 2.2 Hz, 1H), 7.99 (t, J = 2.0 Hz, 1H), | |
| | | 7.96-7.92 (m, 1H), 7.53-7.43 (m, | |
| | | 3H), 7.42 (d, J = 1.8 Hz, 1H), 7.29- | |
| | | 7.26 (m, 2H), 1H), 6.83-6.81 (m, | |
| | | 1H), 6.47-6.45 (m, 1H), 5.34-5.31 | |
| | | (m, 2H), 4.42-4.23 (m, 2H), 4.04- | |
| | | 3.70 (m, 5H), 3.34-3.27 (m, 2H), | |
| | | 3.04-2.82 (m, 4H), 2.19-2.10 (m, | |
| | | 2H). | |
| 159 | 706.3 | ¹H NMR (500 MHz, DMSO-d₆) δ | 3 |
| | | 10.11 (s, 1H), 8.23 (d, J = 2.1 Hz, | |
| | | 1H), 7.97-7.93 (m, 1H), 7.91-7.88 | |
| | | (m, 1H), 7.62 (dd, J = 8.4, 2.1 Hz, | |
| | | 1H), 7.56-7.48 (m, 2H), 7.48-7.43 | |
| | | (m, 2H), 7.42 (d, J = 1.8 Hz, 1H), | |
| | | 7.29-7.26 (m, 2H), 1H), 6.83-6.81 | |
| | | (m, 1H), 6.47-6.45 (m, 1H), 5.34- | |
| | | 5.31 (m, 2H), 4.44-3.97 (m, 4H), | |
| | | 3.93 (s, 1H), 3.90 (s, 3H), 3.87-3.74 | |
| | | (m, 2H), 3.34-3.27 (m, 2H), 3.04- | |
| | | 2.82 (m, 4H), 2.19-2.10 (m, 2H). | |
| 160 | 726.2 | ¹H NMR (500 MHz, DMSO-d₆) δ | 3 |
| | | 8.23 (d, J = 2.1 Hz, 1H), 7.91-7.88 | |
| | | (m, 1H), 7.86-7.82 (m, 2H), 7.53- | |
| | | 7.44 (m, 2H), 7.40 (dd, J = 8.2, 1.3 | |
| | | Hz, 2H), 7.29-7.26 (m, 2H), 1H), | |
| | | 6.83-6.81 (m, 1H), 6.47-6.45 (m, | |
| | | 1H), 5.34-5.31 (m, 2H), 4.43-3.82 | |
| | | (m, 6H), 3.75-3.69 (m, 1H), 3.34- | |
| | | 3.27 (m, 2H), 3.04-2.82 (m, 4H), | |
| | | 2.13-1.92 (m, 2H). | |
| 161 | 746.2 | ¹H NMR (500 MHz, Chloroform-d) δ | 3 |
| | | 12.12 (s, 1H), 11.51 (s, 1H), 8.03 | |
| | | (dd, J = 13.4, 2.2 Hz, 2H), 7.85 (dd, J = | |
| | | 8.7, 2.1 Hz, 1H), 7.62-7.50 (m, | |
| | | 2H), 7.42-7.31 (m, 4H), 7.26-7.13 | |
| | | (m, 2H), 6.83 (dd, J = 7.8, 1.2 Hz, | |
| | | 1H), 6.60-6.45 (m, 1H), 5.39-5.29 | |
| | | (m, 2H), 4.39-4.35 (m, 1H), 4.32- | |
| | | 4.23 (m, 1H), 4.17-4.14 (m, 1H), 4.03- | |
| | | 3.93 (m, 2H), 3.90-3.77 (m, 2H), | |
| | | 3.38-3.27 (m, 2H), 3.10-2.97 (m, | |
| | | 2H), 2.95-2.82 (m, 2H), 2.42-2.24 | |
| | | (m, 1H), 2.15-1.99 (m, 1H). | |
| 162 | 718.3 | ¹H NMR (500 MHz, Chloroform-d) δ | 3 |
| | | 11.49 (s, 1H), 10.35 (s, 1H), 8.07 (d, | |
| | | J = 24.1 Hz, 2H), 7.69-7.59 (m, 2H), | |
| | | 7.58-7.37 (m, 5H), 7.20-7.04 (m, | |
| | | 2H), 6.86-6.80 (m, 1H), 6.37-6.34 | |
| | | (m, 1H), 5.50-5.42 (m, 2H), 4.35- | |
| | | 4.33 (m, 1H), 4.31-4.24 (m, 1H), | |
| | | 4.17-4.14 (m, 1H), 4.05-3.75 (m, | |
| | | 4H), 3.40-3.22 (m, 2H), 3.12-2.82 | |
| | | (m, 4H), 2.43-1.94 (m, 3H), 1.11- | |
| | | 0.87 (m, 4H). | |
| 163 | 786.3 | ¹H NMR (500 MHz, Chloroform-d) δ | 3 |
| | | 12.08 (s, 1H), 11.28 (s, 1H), 8.11- | |
| | | 7.86 (m, 3H), 7.83 (dd, J = 8.7, 2.1 | |
| | | Hz, 1H), 7.24-7.06 (m, 6H), 6.95- | |
| | | 6.39 (m, 2H), 5.46-5.17 (m, 2H), | |
| | | 4.42-4.38 (m, 1H), 4.31-4.24 (m, | |
| | | 1H), 4.17 (dd, J = 15.6, 5.3 Hz, 1H), | |
| | | 4.02-3.69 (m, 4H), 3.38-3.23 (m, | |
| | | 2H), 3.13-2.80 (m, 4H), 2.46-1.93 | |
| | | (m, 3H), 1.17-0.83 (m, 4H). | |
| 164 | 732.3 | ¹H NMR (500 MHz, Chloroform-d) δ | 3 |
| | | 8.09-8.02 (m, 1H), 8.84-7.78 (m, | |
| | | 2H), 7.56-7.40 (m, 5H), 7.22-7.10 | |
| | | (m, 2H), 6.83-6.81 (m, 1H), 6.57- | |
| | | 6.51 (m, 1H), 5.32-5.12 (m, 2H), | |
| | | 4.35 (d, J = 15.5 Hz, 1H), 4.30-4.24 | |
| | | (m, 1H), 4.17-4.13 (m, 1H), 4.03- | |
| | | 3.67 (m, 4H), 3.41-2.92 (m, 6H), | |
| | | 2.69-2.43 (m, 2H), 2.05-1.94 (m, | |
| | | 1H), 1.16-1.10 (m, 2H), 0.99-0.84 | |
| | | (m, 2H). | |
| 165 | 689.3 | ¹H NMR (500 MHz, Chloroform-d) δ | 3 |
| | | 8.37 (d, J = 2.0 Hz, 1H), 8.09 (d, J = | |
| | | 2.1 Hz, 1H), 7.81 (dd, J = 8.8, 2.0 | |
| | | Hz, 1H), 7.60-7.58 (m, 2H), 7.52 (d, | |
| | | J = 8.0 Hz, 1H), 7.48-7.42 (m, 2H), | |
| | | 7.42-7.34 (m, 2H), 6.89 (dd, J = | |
| | | 7.8, 1.2 Hz, 1H), 6.51-6.43 (m, 1H), | |
| | | 5.35 (s, 2H), 4.38-4.31 (m, 1H), 4.24- | |
| | | 4.19 (m, 1H), 4.16-4.10 (m, 1H), | |
| | | 4.00-3.93 (m, 2H), 3.82-3.76 (m, | |
| | | 2H), 3.32-3.28 (m, 2H), 3.03-2.97 | |
| | | (m, 2H), 2.92-2.89 m, 5H), 2.60 (s, | |
| | | 3H), 2.30-2.15 (m, 2H). | |
| 166 | 679.2 | ¹H NMR (500 MHz, Chloroform-d) δ | 3 |
| | | 8.32 (d, J = 2.1 Hz, 1H), 8.07 (d, J = | |
| | | 2.2 Hz, 1H), 7.85-7.80 (m, 1H), 7.78 | |
| | | (d, J = 8.0 Hz, 1H), 7.73-7.68 (m, | |
| | | 1H), 7.42-7.37 (m, 3H), 7.27-7.17 | |
| | | (m, 3H), 6.83-6.81 (m, 1H), 6.59- | |
| | | 6.52 (m, 1H), 5.31-5.24 (m, 2H), | |
| | | 4.32-4.28 (m, 2H), 4.17-4.13 (m, | |
| | | 1H), 3.91-3.79 (m, 4H), 3.32-3.08- | |
| | | 3.02 (m, 4H), 2.91-2.87 (m, 2H), | |
| | | 2.30-2.04 (m, 2H). | |
| 167 | 693.2 | ¹H NMR (500 MHz, Chloroform-d) δ | 3 |
| | | 8.32 (d, J = 2.1 Hz, 1H), 8.07 (d, J = | |
| | | 2.2 Hz, 1H), 7.86-7.80 (m, 1H), 7.78 | |
| | | (d, J = 8.0 Hz, 1H), 7.74-7.68 (m, | |
| | | 1H), 7.42-7.37 (m, 2H), 7.27-7.17 | |
| | | (m, 3H), 6.84 (dd, J = 7.8 1.2 Hz, | |
| | | 1H), 6.59-6.52 (m, 1H), 5.31-5.24 | |
| | | (m, 2H), 4.32-4.28 (m, 2H), 4.17- | |
| | | 4.13 (m, 1H), 3.91-3.79 (m, 4H), | |
| | | 3.32-3.08-3.02 (m, 4H), 2.91-2.87 | |
| | | (m, 2H), 2.74 (s, 3H), 2.30-2.04 (m, | |
| | | 2H). | |
| 168 | 694.2 | ¹H NMR (500 MHz, Chloroform-d) δ | 3 |
| | | 12.15 (s, 1H), 11.36 (s, 1H), 8.64 (s, | |
| | | 1H), 8.15 (d, J = 2.2 Hz, 1H), 7.83 (d, | |
| | | J = 8.8 Hz, 1H), 7.45-7.34 (m, 4H), | |
| | | 7.17-7.14 (m, 2H), 6.86-6.59 (m, | |
| | | 2H), 5.39-5.34 (m, 2H), 4.39-4.29 | |
| | | (m, 2H), 4.17-4.13 (m, 1H), 3.93- | |
| | | 3.87 (m, 2H), 3.85-3.77 (m, 2H), | |
| | | 3.38-3.29 (m, 2H), 3.05-3.02 (m, | |
| | | 2H), 2.93-2.91 (m, 2H), 2.34-2.14 | |
| | | (m, 2H). | |
| 169 | 742.2 | ¹H NMR (500 MHz, Chloroform-d) δ | 3 |
| | | 8.09 (dd, J = 4.0, 2.0 Hz, 2H), 7.85 | |
| | | (dd, J = 8.8, 2.0 Hz, 1H), 7.47-7.45 | |
| | | (m, 2H), 7.44-7.42 (m, 1H), 7.37- | |
| | | 7.34 (m, 2H), 7.21-7.15 (m, 3H), | |
| | | 6.76-6.55 (m, 2H), 5.35-5.34 (m, | |
| | | 2H), 4.33-4.28 (m, 2H), 4.17- | |
| | | 4.11 (m, 1H), 4.01-3.93 (m, 2H), | |
| | | 3.85-3.76 (m, 2H), 3.32-3.21 (m, | |
| | | 2H), 3.08-2.89 (m, 3H), 2.65 (s, 3H), | |
| | | 2.28-2.15 (m, 2H). | |
| 170 | 720.3 | ¹H NMR (500 MHz, Chloroform-d) δ | 3 |
| | | 8.08 (d, J = 2.1 Hz, 1H), 7.81-7.78 | |
| | | (m, 2H), 7.45-7.37 (m, 5H), 7.10- | |
| | | 7.05 (m, 2H), 6.83 (dd, J = 7.8, 1.2 | |
| | | Hz, 1H), 6.65-6.58 (m, 1H), 5.31- | |
| | | 5.24 (m, 2H), 4.39-4.37 (m, 1H), | |
| | | 4.25-4.19 (m, 2H), 4.05 (d, J = 13.7 | |
| | | Hz, 1H), 3.93-3.89 (m, 2H), 3.76- | |
| | | 3.68 (m, 1H), 3.32-3.35 (m, 2H), 3.08- | |
| | | 2.88 (m, 9H), 2.25-2.15 (m, 2H), | |
| | | 1.35 (t, J = 7.6 Hz, 3H). | |
| 171 | 732.3 | ¹H NMR (500 MHz, Chloroform-d) δ | 3 |
| | | 12.18 (s, 2H), 11.02 (s, 2H), 8.05 (d, | |
| | | J = 2.1 Hz, 2H), 7.86-7.80 (m, 4H), | |
| | | 7.50 (d, J = 2.2 Hz, 2H), 7.45-7.39 | |
| | | (m, 8H), 7.17-7.14 (m, 4H), 6.86- | |
| | | 6.55 (m, 4H), 5.31-5.24 (m, 4H), | |
| | | 4.30-4.25 (m, 4H), 4.17-4.12 (m, | |
| | | 2H), 4.00-3.93 (m, 4H), 3.82-3.76 | |
| | | (m, 2H), 3.50-3.44 (m, 2H), 3.32- | |
| | | 3.03 (m, 8H), 2.99-2.84 (m, 10H), | |
| | | 2.28-2.17 (m, 4H), 1.14-1.06 (m, | |
| | | 8H). | |
| 172 | 742.2 | ¹H NMR (500 MHz, Chloroform-d) δ | 3 |
| | | 8.09 (d, J = 2.1 Hz, 1H), 7.83-7.79 | |
| | | (m, 3H), 7.40-7.32 (m, 5H), 7.17- | |
| | | 7.14 (m, 2H), 6.86-6.55 (m, 2H), | |
| | | 5.32-5.24 (m, 2H), 4.31-4.28 (m, | |
| | | 2H), 4.17-4.12 (m, 1H), 4.05 (d, J = | |
| | | 13.7 Hz, 1H), 3.93-3.85 (m, 2H), | |
| | | 3.76-3.71 (m, 1H), 3.32-3.08 (m, | |
| | | 2H), 3.02-2.84 (m, 6H), 2.27-2.19 | |
| | | (m, 2H). | |
| 173 | 720.3 | ¹H NMR (500 MHz, Chloroform-d) δ | 3 |
| | | 11.55 (s, 1H), 9.65 (s, 1H), 8.07 (d, J = | |
| | | 2.1 Hz, 1H), 7.85 (dd, J = 8.8, 2.4 | |
| | | Hz, 1H), 7.45-7.34 (m, 5H), 7.17- | |
| | | 7.14 (m, 2H), 6.86-6.55 (m, 2H), | |
| | | 5.31-5.24 (m, 2H), 4.35-4.28 (m, | |
| | | 2H), 4.17-4.16 (m, 1H), 4.01 (d, J = | |
| | | 13.7 Hz, 1H), 3.93-3.85 (m, 2H), | |
| | | 3.86-3.75 (m, 1H), 3.32-3.37 (m, | |
| | | 2H), 3.04-2.89 (m, 3H), 2.75 (d, J = | |
| | | 0.8 Hz, 3H), 2.51 (s, 3H), 2.35-2.27 | |
| | | (m, 2H). | |
| 174 | 710.2 | ¹H NMR (500 MHz, Chloroform-d) δ | 3 |
| | | 8.06 (d, J = 2.1 Hz, 1H), 7.98 (d, J = | |
| | | 4.9 Hz, 1H), 7.85 (dd, J = 8.8, 2.0 | |
| | | Hz, 1H), 7.47 (t, J = 7.9 Hz, 1H), | |
| | | 7.42-7.35 (m, 3H), 7.33 (d, J = 8.0 | |
| | | Hz, 1H), 7.17-7.14 (m, 2H), 6.86- | |
| | | 6.83 (m, 1H), 6.55-6.51 (m, 1H), 5.32- | |
| | | 5.29 (m, 2H), 4.32-4.28 (m, 2H), | |
| | | 4.17-4.11 (m, 1H), 4.00-3.93 (m, | |
| | | 2H), 3.82-3.76 (m, 2H), 3.36- | |
| | | 3.31 (m, 2H), 3.01-2.83 (m, 7H), 2.27- | |
| | | 2.15 (m, 2H). | |
| 175 | 725.2 | ¹H NMR (500 MHz, Chloroform-d) δ | 3 |
| | | 8.09 (d, J = 2.1 Hz, 1H), 7.82-7.76 | |
| | | (M, 1H), 7.63 (d, J = 4.9 Hz, 1H), | |
| | | 7.51-7.37 (m, 4H), 7.22-7.16 (m, | |
| | | 2H), 6.80-6.57 (m, 2H), 5.31-5.20 | |
| | | (m, 2H), 4.55-4.38 (m, 2H), 4.17- | |
| | | 4.11 (m, 1H), 4.03 (d, J = 13.7 Hz, | |
| | | 1H), 3.95-3.88 (m, 2H), 3.77-3.71 | |
| | | (m, 1H), 3.27-3.02 (m, 3H), 3.00- | |
| | | 2.95 (m, 3H), 2.65 (s, 3H), 2.30- | |
| | | 2.19 (m, 2H). | |
| 176 | 710.2 | ¹H NMR (500 MHz, Chloroform-d) δ | 3 |
| | | 12.12 (s, 1H), 10.48 (s, 1H), 8.07 (d, | |
| | | J = 2.1 Hz, 1H), 7.80-7.71 (m, 2H), | |
| | | 7.42-7.33 (m, 5H), 7.17-7.12 (m, | |
| | | 2H), 6.85-6.80 (m, 1H), 6.55-6.48 | |
| | | (m, 1H), 5.39-5.29 (m, 2H), 4.31- | |
| | | 4.28 (m, 2H), 4.17-4.11 (m, 1H), | |
| | | 4.03-3.93 (m, 2H), 3.81-3.76 (m, | |
| | | 2H), 3.32-3.28 (m, 2H), 3.04-2.90 | |
| | | (m, 4H), , 2.30-2.19 (m, 2H). | |
| 177 | 701.3 | ¹H NMR (500 MHz, Chloroform-d) δ | 3 |
| | | 8.09 (d, J = 2.0 Hz, 1H), 7.88-7.78 | |
| | | (m, 2H), 7.50-7.37 (m, 7H), 6.88 (d, | |
| | | J = 7.9 Hz, 1H), 6.58-6.51 (m, 1H), | |
| | | 5.33-5.29 (m, 2H), 4.39-4.31 (m, | |
| | | 2H), 4.19-4.11 (m, 1H), 4.03-3.96 | |
| | | (m, 1H), 3.87-3.76 (m, 2H), 3.35- | |
| | | 3.31 (m, 2H), 3.11-2.90 (m, 7H) | |
| | | 2.25-2.15 (m, 2H). | |
| 178 | 724.3 | ¹H NMR (500 MHz, Chloroform-d) δ | 3 |
| | | 10.78 (s, 1H), 9.95 (s, 1H), 8.09 (d, J = | |
| | | 2.2 Hz, 1H), 7.88-7.86 (m, 1H), | |
| | | 7.63 (d, J = 2.2 Hz, 1H), 7.55 (d, J = | |
| | | 2.1 Hz, 1H), 7.51-7.37 (m, 6H), | |
| | | 6.88 (d, J = 7.9 Hz, 1H), 6.58-6.51 | |
| | | (m, 1H), 5.31-5.29 (m, 2H), 4.39- | |
| | | 4.30 (m, 2H), 4.19-4.11 (m, 1H), 3.97- | |
| | | 3.85 (m, 4H), 3.35-3.28 (m, 2H), | |
| | | 3.18-3.00 (m, 4H), 2.39-2.33 (m, | |
| | | 1H), 2.21 (s, 3H), 2.18-2.13 (m, | |
| | | 3H), 1.69-1.63 (m, 2H). | |
| 179 | 728.3 | ¹H NMR (500 MHz, Chloroform-d) δ | 3 |
| | | 10.31 (s, 1H), 8.35 (s, 1H), 8.09 (d, J = | |
| | | 2.2 Hz, 1H), 7.88-7.86 (m, 1H), | |
| | | 7.58-7.43 (m, 8H), 6.88 (d, J = 7.9 | |
| | | Hz, 1H), 6.58-6.51 (m, 1H), 5.31- | |
| | | 5.29 (m, 2H), 4.39-4.30 (m, 2H), | |
| | | 4.19-4.11 (m, 1H), 3.97-3.85 (m, | |
| | | 4H), 3.35-3.28 (m, 2H), 3.18-3.00 | |
| | | (m, 4H), 2.39-2.33 (m, 1H), 2.20- | |
| | | 2.14 (m, 3H), 1.71-1.63 (m, 2H). | |
| 180 | 687.3 | ¹H NMR (500 MHz, Chloroform-d) δ | 3 |
| | | 8.28 (d, J = 1.7 Hz, 1H), 8.09 (d, J = | |
| | | 2.2 Hz, 1H), 7.89-7.82 (m, 1H), 7.88- | |
| | | 7.86 (m, 1H), 7.66 (d, J = 8.1 Hz, | |
| | | 1H), 7.54-7.37 (m, 6H), 6.88 (d, J = | |
| | | 7.9 Hz, 1H), 6.58-6.51 (m, 1H), 5.31- | |
| | | 5.29 (m, 2H), 4.39-4.30 (m, 2H), | |
| | | 4.19-4.11 (m, 1H), 3.97-3.85 (m, | |
| | | 4H), 3.35-3.28 (m, 2H), 3.18-3.00 | |
| | | (m, 4H), 2.39-2.33 (m, 1H), 2.06- | |
| | | 2.01 (m, 1H). | |
| 181 | 697.3 | ¹H NMR (500 MHz, Chloroform-d) δ | 3 |
| | | 11.72 (s, 1H), 10.99 (s, 1H), 8.09 (d, | |
| | | J = 2.0 Hz, 1H), 7.80 (d, J = 8.8 Hz, | |
| | | 1H), 7.69-7.50 (m, 5H), 7.42-7.33 | |
| | | (m, 3H), 6.88 (d, J = 7.9 Hz, 1H), | |
| | | 6.58-6.51 (m, 1H), 5.33-5.29 (m, | |
| | | 2H), 4.39-4.31 (m, 2H), 4.19-4.11 | |
| | | (m, 1H), 4.03-3.96 (m, 1H), 3.87- | |
| | | 3.76 (m, 2H), 3.35-3.31 (m, 2H), 3.05- | |
| | | 2.98 (m, 2H), 2.91 (s, 3H), 2.88- | |
| | | 2.85 (m, 2H), 2.65 (s, 3H), 2.25- | |
| | | 2.15 (m, 2H). | |
| 182 | 710.3 | ¹H NMR (500 MHz, Chloroform-d) δ | 3 |
| | | 9.98 (s, 1H), 9.49 (s, 1H), 8.09 (d, J = | |
| | | 2.0 Hz, 1H), 7.88-7.86 (m, 1H), 7.68 | |
| | | (d, J = 2.0 Hz, 1H), 7.62-7.59 (m, | |
| | | 1H), 7.51-7.37 (m, 6H), 7.29 (s, | |
| | | 1H), 6.88 (d, J = 7.9 Hz, 1H), 6.58- | |
| | | 6.51 (m, 1H), 5.31-5.29 (m, 2H), | |
| | | 4.39-4.30 (m, 2H), 4.19-4.11 (m, | |
| | | 1H), 3.97-3.85 (m, 4H), 3.35-3.28 | |
| | | (m, 2H), 3.18-3.00 (m, 4H), 2.39- | |
| | | 2.33 (m, 1H), 2.20-2.11 (m, 2H), | |
| | | 2.07-2.03 (m, 1H), 1.70-1.64 (m, | |
| | | 2H). | |
| 183 | 733.3 | ¹H NMR (500 MHz, Chloroform-d) δ | 3 |
| | | 8.04 (d, J = 4.0 Hz, 2H), 7.80 (d, J = | |
| | | 8.8 Hz, 1H), 7.51-7.33 (m, 7H), | |
| | | 7.17 (s, 1H), 6.88 (d, J = 8.0 Hz, 1H), | |
| | | 6.58-6.51 (m, 1H), 5.31-5.29 (m, | |
| | | 2H), 4.39-4.31 (m, 2H), 4.19-4.11 | |
| | | (m, 1H), 4.03-3.96 (m, 1H), 3.87- | |
| | | 3.76 (m, 2H), 3.35-3.30 (m, 2H), 3.03- | |
| | | 2.94 (m, 2H), 2.88-2.85 (m, 2H), | |
| | | 2.71 (s, 3H), 2.25-2.15 (m, 2H). | |
| 184 | 737.3 | ¹H NMR (500 MHz, Chloroform-d) δ | 3 |
| | | 12.02 (s, 1H), 11.31 (s, 1H), 8.05- | |
| | | 8.03 (m, 2H), 7.80 (d, J = 8.8 Hz, | |
| | | 1H), 7.64-7.55 (m, 2H), 7.51-7.37 | |
| | | (m, 6H), 6.88 (d, J = 7.9 Hz, 1H), | |
| | | 6.58-6.51 (m, 1H), 5.31-5.29 (m, | |
| | | 2H), 4.39-4.31 (m, 2H), 4.19-4.11 | |
| | | (m, 1H), 4.03-3.96 (m, 1H), 3.87- | |
| | | 3.76 (m, 2H), 3.35-3.31 (m, 2H), 3.08- | |
| | | 2.97 (m, 2H), 2.88-2.85 (m, 2H), | |
| | | 2.25-2.15 (m, 2H). | |
| 185 | 723.3 | ¹H NMR (500 MHz, Chloroform-d) δ | 3 |
| | | 8.09 (d, J = 2.0 Hz, 1H), 7.86 (d, J = | |
| | | 2.2 Hz, 1H), 7.80 (d, J = 8.8 Hz, 1H), | |
| | | 7.55-7.39 (m, 8H), 6.88 (d, J = 7.9 | |
| | | Hz, 1H), 6.58-6.51 (m, 1H), 5.33- | |
| | | 5.29 (m, 2H), 4.39-4.31 (m, 2H), | |
| | | 4.19-4.11 (m, 1H), 4.03-3.96 (m, | |
| | | 1H), 3.87-3.76 (m, 2H), 3.35-3.31 | |
| | | (m, 2H), 3.03-2.94 (m, 2H), 2.88- | |
| | | 2.85 (m, 2H), 2.65 (s, 3H), 2.31- | |
| | | 2.19 (m, 3H), 1.17-0.92 (m 5H). | |
| 186 | 697.3 | ¹H NMR (500 MHz, Chloroform-d) δ | 3 |
| | | 11.96 (s, 1H), 10.56 (s, 1H), 8.09 (d, | |
| | | J = 2.0 Hz, 1H), 7.88 (d, J = 2.2 Hz, | |
| | | 1H), 7.80 (d, J = 8.8 Hz, 1H), 7.64- | |
| | | 7.60 (m, 1H), 7.49-7.37 (m, 7H), | |
| | | 6.88 (d, J = 7.9 Hz, 1H), 6.58-6.51 | |
| | | (m, 1H), 5.33-5.29 (m, 2H), 4.39- | |
| | | 4.31 (m, 2H), 4.19-4.11 (m, 1H), | |
| | | 4.03-3.96 (m, 1H), 3.87-3.76 (m, | |
| | | 2H), 3.35-3.31 (m, 2H), 3.03-2.94 | |
| | | (m, 2H), 2.88-2.85 (m, 2H), 2.78- | |
| | | 2.71 (m, 2H), 2.31-2.15 (m, 2H), | |
| | | 1.36 (t, J = 7.6 Hz, 3H). | |
| 187 | 683.3 | ¹H NMR (500 MHz, Chloroform-d) δ | 3 |
| | | 12.32 (s, 1H), 10.67 (s, 1H), 8.09 (d, | |
| | | J = 2.0 Hz, 1H), 7.89 (d, J = 2.2 Hz, | |
| | | 1H), 7.80 (d, J = 8.8 Hz, 1H), 7.69- | |
| | | 7.67 (m, 1H), 7.51-7.37 (m, 8H), | |
| | | 6.88 (d, J = 7.9 Hz, 1H), 6.58-6.51 | |
| | | (m, 1H), 5.33-5.29 (m, 2H), 4.39- | |
| | | 4.31 (m, 2H), 4.19-4.11 (m, 1H), | |
| | | 4.03-3.96 (m, 1H), 3.87-3.76 (m, | |
| | | 2H), 3.35-3.31 (m, 2H), 3.03-2.94 | |
| | | (m, 2H), 2.95 (s, 3H), 2.93-2.84 (m, | |
| | | 2H), 2.25-2.15 (m, 2H). | |
| 188 | 683.3 | ¹H NMR (500 MHz, Chloroform-d) δ | 3 |
| | | 11.79 (s, 1H), 10.75 (s, 1H), 8.16 (d, | |
| | | J = 1.7 Hz, 1H), 8.09 (d, J = 2.0 Hz, | |
| | | 1H), 7.86-7.80 (m, 2H), 7.52 (d, J = | |
| | | 2.2 Hz, 1H), 7.51-7.37 (m, 6H), | |
| | | 6.88 (d, J = 7.9 Hz, 1H), 6.58-6.51 | |
| | | (m, 1H), 5.33-5.29 (m, 2H), 4.39- | |
| | | 4.31 (m, 2H), 4.19-4.11 (m, 1H), | |
| | | 4.03-3.96 (m, 1H), 3.87-3.76 (m, | |
| | | 2H), 3.35-3.31 (m, 2H), 3.03-2.94 | |
| | | (m, 2H), 2.88-2.85 (m, 2H), 2.68 (s, | |
| | | 3H), 2.25-2.15 (m, 2H). | |
| 189 | 693.2 | ¹H NMR (500 MHz, Chloroform-d) δ | 3 |
| | | 11.77 (s, 1H), 11.47 (s, 1H), 8.81- | |
| | | 8.78 (m, 1H), 8.67 (d, J = 1.7 Hz, | |
| | | 1H), 8.09 (d, J = 2.2 Hz, 1H), 7.88- | |
| | | 7.86 (m, 1H), 7.45 (t, J = 7.9 Hz, | |
| | | 1H), 7.42-7.34 (m, 3H), 7.18-7.11 | |
| | | (m, 2H), 6.88 (d, J = 7.9 Hz, 1H), | |
| | | 6.58-6.51 (m, 1H), 5.32-5.24 (m, | |
| | | 2H), 4.39-4.30 (m, 2H), 4.19-4.11 | |
| | | (m, 1H), 3.97-3.85 (m, 4H), 3.35- | |
| | | 3.28 (m, 2H), 3.05-2.87 (m, 7H), | |
| | | 2.39-2.33 (m, 1H), 2.06-2.01 (m, | |
| | | 1H). | |
| 190 | 694.2 | ¹H NMR (500 MHz, Chloroform-d) δ | 3 |
| | | 8.69 (s, 1H), 8.07 (d, J = 2.2 Hz, 1H), | |
| | | 7.85 (dd, J = 8.7, 2.1 Hz, 1H), 7.47- | |
| | | 7.39 (m, 4H),, 7.18-7.11 (m, 2H), | |
| | | 6.88 (d, J = 7.9 Hz, 1H), 6.58-6.51 | |
| | | (m, 1H), 5.32-5.24 (m, 2H), 4.39- | |
| | | 4.30 (m, 2H), 4.19-4.11 (m, 1H), 3.97- | |
| | | 3.85 (m, 4H), 3.35-3.28 (m, 2H), | |
| | | 3.08-3.02 (m, 1H), 2.99 (ddt, J = | |
| | | 7.0, 3.9, 1.0 Hz, 1H), 2.88-2.85 (m, | |
| | | 2H), 2.39-2.33 (m, 1H), 2.06-2.01 | |
| | | (m, 1H). | |
| 191 | 693.2 | ¹H NMR (500 MHz, Chloroform-d) δ | 3 |
| | | 8.14 (d, J = 2.2 Hz, 1H), 7.88-7.85 | |
| | | (m, 2H), 7.73 (d, J = 7.6 Hz, 1H), | |
| | | 7.47-7.39 (m, 4H),, 7.18-7.11 (m, | |
| | | 2H), 6.88 (d, J = 7.9 Hz, 1H), 6.58- | |
| | | 6.51 (m, 1H), 5.32-5.24 (m, 2H), | |
| | | 4.39-4.30 (m, 2H), 4.19-4.11 (m, | |
| | | 1H), 3.97-3.85 (m, 4H), 3.35-3.28 | |
| | | (m, 2H), 3.07-3.00 (m, 2H), 2.88- | |
| | | 2.85 (m, 2H), 2.39-2.33 (m, 1H), | |
| | | 2.06-2.01 (m, 1H). | |
| 192 | 693.3 | ¹H NMR (500 MHz, Chloroform-d) δ | |
| | | 8.18 (d, J = 2.2 Hz, 1H), 7.88-7.85 | |
| | | (m, 2H), 7.73 (d, J = 7.6 Hz, 1H), | |
| | | 7.45-7.39 (m, 4H),, 7.18-7.11 (m, | |
| | | 2H), 6.88 (d, J = 7.9 Hz, 1H), 6.58- | |
| | | 6.51 (m, 1H), 5.32-5.24 (m, 2H), | |
| | | 4.39-4.30 (m, 2H), 4.19-4.11 (m, | |
| | | 1H), 3.97-3.85 (m, 4H), 3.35-3.28 | |
| | | (m, 2H), 3.07-3.00 (m, 2H), 2.88- | |
| | | 2.85 (m, 2H), 2.39-2.33 (m, 1H), | |
| | | 2.06-2.01 (m, 1H). | |
| 193 | 681.2 | ¹H NMR (500 MHz, Chloroform-d) δ | 3 |
| | | 8.23 (d, J = 2.0 Hz, 1H), 8.09 (d, J = | |
| | | 2.2 Hz, 1H), 7.88-7.86 (m, 1H), 7.76- | |
| | | 7.71 (m, 2H), 7.48-7.42 (m, 1H), 7.40- | |
| | | 7.37 (m, 1H), 7.21-7.10 (m, 3H), | |
| | | 6.79 (d, J = 7.7 Hz, 1H), 5.31-5.30 (m, | |
| | | 2H), 4.39-4.30 (m, 2H), 4.19-4.11 | |
| | | (m, 1H), 3.85-3.70 (m, 4H), 3.01- | |
| | | 2.77 (m, 3H), 2.68-2.61 (m, 2H), | |
| | | 2.39-2.33 (m, 1H), 2.10-1.95 (m, | |
| | | 6H). | |
| 194 | 695.3 | ¹H NMR (500 MHz, Chloroform-d) δ | 3 |
| | | 11.60 (s, 1H), 8.09 (d, J = 2.2 Hz, | |
| | | 1H), 8.01 (d, J = 2.2 Hz, 1H), 7.88- | |
| | | 7.86 (m, 1H), 7.55 (d, J = 2.2 Hz, | |
| | | 1H), 7.48-7.42 (m, 1H), 7.42-7.38 | |
| | | (m, 2H), 7.21-7.10 (m, 3H), 6.79 (d, J = | |
| | | 7.7 Hz, 1H), 5.31-5.30 (m, 2H), | |
| | | 4.39-4.30 (m, 2H), 4.19-4.11 (m, | |
| | | 1H), 3.85-3.70 (m, 4H), 3.01-2.77 | |
| | | (m, 3H), 2.68 (s, 3H), 2.65 (ddd, J = | |
| | | 12.5, 8.8, 6.4 Hz, 2H), 2.39-2.33 (m, | |
| | | 1H), 2.10-1.95 (m, 6H). | |
| 195 | 696.3 | ¹H NMR (500 MHz, Chloroform-d) δ | 3 |
| | | 11.95 (s, 1H), 11.76 (s, 1H), 8.68 (s, | |
| | | 1H), 8.09 (d, J = 2.2 Hz, 1H), 7.85 | |
| | | (dd, J = 8.8, 2.2 Hz, 1H), 7.48-7.42 | |
| | | (m, 1H), 7.42-7.38 (m, 2H), 7.21- | |
| | | 7.10 (m, 3H), 6.79 (d, J = 7.7 Hz, 1H), | |
| | | 5.31-5.30 (m, 2H), 4.39-4.30 (m, | |
| | | 2H), 4.19-4.11 (m, 1H), 3.85-3.70 | |
| | | (m, 4H), 3.01-2.77 (m, 3H), 2.68- | |
| | | 2.61 (m, 2H), 2.39-2.33 (m, 1H), 2.08- | |
| | | 1.93 (m, 6H). | |
| 196 | 710.2 | ¹H NMR (500 MHz, Chloroform-d) δ | 4 |
| | | 11.69 (s, 1H), 10.51 (s, 1H), 8.23 (d, | |
| | | J = 2.2 Hz, 1H), 7.99 (d, J = 1.6 Hz, | |
| | | 1H), 7.75-7.73 (m, 1H), 7.57-7.51 | |
| | | (m, 1H), 7.46-7.39 (m, 4H), 7.19- | |
| | | 7.13 (m, 2H), 6.88 (d, J = 7.9 Hz, | |
| | | 1H), 6.58-6.51 (m, 1H), 5.32-5.24 | |
| | | (m, 2H), 4.33-4.21 (m, 2H), 4.02- | |
| | | 3.93 (m, 3H), 3.89-3.78 (m, 2H), | |
| | | 3.35-3.28 (m, 2H), 3.03-2.97 (m, | |
| | | 2H), 2.91-2.84 (m, 5H), 2.31-2.15 | |
| | | (m, 2H). | |
| 197 | 706.3 | ¹H NMR (500 MHz, Chloroform-d) δ | 4 |
| | | 11.47 (s, 1H), 10.33 (s, 1H), 8.44 (d, | |
| | | J = 2.2 Hz, 1H), 7.97 (d, J = 2.0 Hz, | |
| | | 1H), 7.82 (d, J = 2.1 Hz, 1H), 7.57- | |
| | | 7.51 (m, 1H), 7.46-7.39 (m, 4H), | |
| | | 7.19-7.13 (m, 2H), 6.88 (d, J = 7.9 | |
| | | Hz, 1H), 6.58-6.51 (m, 1H), 5.32- | |
| | | 5.24 (m, 2H), 4.33-4.21 (m, 2H), | |
| | | 4.02-3.93 (m, 3H), 3.89-3.78 (m, | |
| | | 2H), 3.35-3.28 (m, 2H), 3.05-2.95 | |
| | | (m, 5H), 2.88-2.95 (m, 2H), 2.68 (s, | |
| | | 3H), 2.31-2.18 (m, 2H). | |
| 198 | 675.3 | ¹H NMR (500 MHz, Chloroform-d) δ | 4 |
| | | 8.17-7.8.10 (m, 2H), 7.82-7.78 (m, | |
| | | 1H), 7.66-7.63 (m, 1H), 7.47-7.42 | |
| | | (m, 4H), 7.20-7.13 (m, 2H), 6.89- | |
| | | 6.84 (m, 1H), 6.58-6.53 (m, 1H), | |
| | | 5.35-5.24 (m, 2H), 4.39-4.13 (m, | |
| | | 3H), 4.00-3.70 (m, 6H), 3.36-3.28 | |
| | | (m, 2H), 3.08-2.90 (m, 5H), 2.95- | |
| | | 2.84 (m, 2H), 2.39-2.09 (m, 2H). | |
| 199 | 692.3 | ¹H NMR (500 MHz, Chloroform-d) δ | 4 |
| | | 11.81 (s, 1H), 10.29 (s, 1H), 8.15 (d, | |
| | | J = 1.7 Hz, 1H), 7.99-7.94 (m, 1H), | |
| | | 7.90-7.87 (m, 1H), 7.80-7.65 (m, | |
| | | 2H), 7.46-7.39 (m, 4H), 7.19-7.13 | |
| | | (m, 2H), 6.88 (d, J = 7.9 Hz, 1H), | |
| | | 6.58-6.51 (m, 1H), 5.32-5.24 (m, | |
| | | 2H), 4.33-4.28 (m, 2H), 4.05-4.01 | |
| | | (m, 1H), 3.97-3.58 (m, 4H), 3.35- | |
| | | 3.28 (m, 2H), 3.18-3.00 (m, 4H), | |
| | | 2.63 (s, 3H), 2.39-2.35 (m, 1H), 2.06- | |
| | | 2.01 (m, 1H). | |
| 200 | 720.3 | ¹H NMR (500 MHz, Chloroform-d) δ | 4 |
| | | 8.52 (d, J = 2.0 Hz, 1H), 7.99-7.96 | |
| | | (m, 1H), 7.90-7.81 (m, 2H), 7.58- | |
| | | 7.52 (m, 1H), 7.46-7.39 (m, 4H), | |
| | | 7.19-7.13 (m, 2H), 6.88 (d, J = 7.9 | |
| | | Hz, 1H), 6.58-6.51 (m, 1H), 5.32- | |
| | | 5.24 (m, 2H), 4.33-4.28 (m, 2H), | |
| | | 4.12-4.08 (m, 1H), 3.97-3.85 (m, | |
| | | 4H), 3.35-3.28 (m, 2H), 3.10-2.97 | |
| | | (m, 3H), 2.88-2.85 (m, 2H), 2.39- | |
| | | 2.35 (m, 1H), 2.07-2.03 (m, 1H), 1.65 | |
| | | (s, 2H), 1.66 (d, J = 6.6 Hz, 3H). | |
| 201 | 708.2 | ¹H NMR (500 MHz, Chloroform-d) δ | 4 |
| | | 8.45 (d, J = 2.0 Hz, 1H), 7.99-7.96 | |
| | | (m, 1H), 7.88 (dd, J = 7.8, 2.0 Hz, | |
| | | 1H), 7.70-7.66 (m, 1H), 7.58-7.52 | |
| | | (m, 1H), 7.46-7.39 (m, 4H), 7.19- | |
| | | 7.13 (m, 2H), 6.88 (d, J = 7.9 Hz, | |
| | | 1H), 6.58-6.51 (m, 1H), 5.32-5.24 | |
| | | (m, 2H), 4.33-4.28 (m, 2H), 4.14 (s, | |
| | | 3H), 4.05-3.85 (m, 5H), 3.35-3.28 | |
| | | (m, 2H), 3.07-3.00 (m, 2H), 2.88- | |
| | | 2.85 (m, 2H), 2.36-2.31 (m, 1H), | |
| | | 2.06-2.01 (m, 1H). | |
| 202 | 696.2 | ¹H NMR (500 MHz, Chloroform-d) δ | 4 |
| | | 12.37 (s, 1H), 10.87 (s, 1H), 8.57- | |
| | | 8.54 (m, 1H), 7.99-7.96 (m, 1H), | |
| | | 7.93 (dd, J = 7.8, 2.0 Hz, 1H), 7.75 | |
| | | (d, J = 7.9 Hz, 1H), 7.58-7.52 (m, | |
| | | 1H), 7.46-7.39 (m, 4H), 7.19-7.13 | |
| | | (m, 2H), 6.88 (d, J = 7.9 Hz, 1H), | |
| | | 6.58-6.51 (m, 1H), 5.32-5.24 (m, | |
| | | 2H), 4.33-4.28 (m, 2H), 4.12-4.08 | |
| | | (m, 1H), 3.97-3.85 (m, 4H), 3.35- | |
| | | 3.28 (m, 2H), 3.05-3.00 (m, 2H), | |
| | | 2.88-2.85 (m, 2H), 2.39-2.35 (m, | |
| | | 1H), 2.06-2.01 (m, 1H). | |
| 203 | 719.3 | ¹H NMR (500 MHz, Chloroform-d) δ | 4 |
| | | 10.99 (s, 2H), 9.52 (s, 2H), 8.23 (d, J = | |
| | | 2.0 Hz, 2H), 7.95-7.91 (m, 4H), | |
| | | 7.51-7.48 (m, 2H), 7.46-7.40 (m, | |
| | | 8H), 7.17-7.11 (m, 6H), 6.88-6.81 | |
| | | (m, 2H), 6.57-6.53 (m, 2H), 5.33- | |
| | | 5.24 (m, 4H), 4.34-4.28 (m, 4H), | |
| | | 4.06-3.83 (m, 6H), 3.80-3.75 (m, | |
| | | 4H), 3.36-3.32 (m, 4H), 3.05-2.84 | |
| | | (m, 8H), 2.30-2.22 (m, 6H), 2.01- | |
| | | 1.72 (m, 6H). | |
| 204 | 678.2 | ¹H NMR (500 MHz, Chloroform-d) δ | 4 |
| | | 8.30 (d, J = 1.7 Hz, 1H), 7.90 (d, J = | |
| | | 2.0 Hz, 1H), 7.92-7.89 (m, 2H), | |
| | | 7.66 (d, J = 8.2 Hz, 1H), 7.57-7.51 | |
| | | (m, 1H), 7.46-7.39 (m, 4H), 7.19- | |
| | | 7.13 (m, 2H), 6.88 (d, J = 7.9 Hz, | |
| | | 1H), 6.58-6.51 (m, 1H), 5.32-5.24 | |
| | | (m, 2H), 4.36-4.29 (m, 2H), 4.02- | |
| | | 3.93 (m, 3H), 3.87-3.76 (m, 2H), | |
| | | 3.35-3.31 (m, 2H), 3.03-2.94 (m, | |
| | | 2H), 2.88-2.85 (m, 2H), 2.25-2.15 | |
| | | (m, 2H). | |
| 205 | 696.2 | ¹H NMR (500 MHz, Chloroform-d) δ | 4 |
| | | 8.14 (d, J = 1.7 Hz, 1H), 8.04-8.02 | |
| | | (m, 1H), 7.83-7.77 (m, 2H), 7.58- | |
| | | 7.52 (m, 1H), 7.46-7.39 (m, 4H), | |
| | | 7.19-7.13 (m, 2H), 6.88 (d, J = 7.9 | |
| | | Hz, 1H), 6.58-6.51 (m, 1H), 5.32- | |
| | | 5.24 (m, 2H), 4.33-4.28 (m, 2H), | |
| | | 4.12-4.08 (m, 1H), 3.97-3.85 (m, | |
| | | 4H), 3.35-3.28 (m, 2H), 3.05-3.00 | |
| | | (m, 2H), 2.88-2.85 (m, 2H), 2.39- | |
| | | 2.35 (m, 1H), 2.06-2.01 (m, 1H). | |
| 206 | 712.2 | ¹H NMR (500 MHz, Chloroform-d) δ | 4 |
| | | 10.99 (s, 1H), 8.97 (d, J = 7.1 Hz, | |
| | | 1H), 8.38 (d, J = 1.6 Hz, 1H), 8.03 (s, | |
| | | 1H), 7.97 (d, J = 2.0 Hz, 1H), 7.87 | |
| | | (dd, J = 7.1, 1.2 Hz, 1H), 7.57-7.51 | |
| | | (m, 1H), 7.46-7.39 (m, 4H), 7.19- | |
| | | 7.13 (m, 2H), 6.88 (d, J = 7.9 Hz, | |
| | | 1H), 6.58-6.51 (m, 1H), 5.32-5.24 | |
| | | (m, 2H), 4.33-4.21 (m, 2H), 4.02- | |
| | | 3.93 (m, 3H), 3.89-3.78 (m, 2H), | |
| | | 3.35-3.28 (m, 2H), 3.03-2.97 (m, | |
| | | 2H), 2.88-2.85 (m, 2H), 2.31-2.15 (m, | |
| | | 2H). | |
| 207 | 676.2 | ¹H NMR (500 MHz, DMSO-d₆) δ | 4 |
| | | 8.57 (s, 1H), 7.95 (d, J = 2.2 Hz, 1H), | |
| | | 7.66-7.56 (m, 2H), 7.53-7.40 (m, | |
| | | 5H), 7.29-7.26 (m, 2H), 1H), 6.83- | |
| | | 6.81 (m, 1H), 6.47-6.45 (m, 1H), | |
| | | 5.34-5.31 (m, 2H), 4.42-4.24 (m, | |
| | | 2H), 4.02-3.70 (m, 5H), 3.34-3.27 | |
| | | (m, 2H), 3.04-2.82 (m, 4H), 2.19- | |
| | | 2.10 (m, 2H) | |
| 208 | 737.2 | ¹H NMR (500 MHz, Chloroform-d) δ | 4 |
| | | 10.31 (s, 2H), 8.28 (s, 2H), 7.95 (dd, | |
| | | J = 16.1, 2.1 Hz, 4H), 7.79 (dd, J = | |
| | | 8.1, 2.2 Hz, 2H), 7.51-7.48 (m, 2H), | |
| | | 7.46-7.40 (m, 8H), 7.21-7.13 (m, | |
| | | 3H), 6.88-6.81 (m, 2H), 6.57-6.53 | |
| | | (m, 2H), 5.33-5.24 (m, 4H), 4.34- | |
| | | 4.25 (m, 4H), 4.06-3.83 (m, 6H), | |
| | | 3.80-3.75 (m, 4H), 3.35-3.31 (m, | |
| | | 4H), 3.05-2.84 (m, 8H), 2.30-2.22 | |
| | | (m, 6H), 2.14-2.08 (m, 2H), 1.78- | |
| | | 1.64 (m, 4H). | |
| 209 | 733.3 | ¹H NMR (500 MHz, Chloroform-d) δ | 4 |
| | | 11.36 (s, 1H), 8.97 (s, 1H), 7.95-7.93 | |
| | | (m, 2H), 7.85 (d, J = 2.1 Hz, 1H), | |
| | | 7.58-7.52 (m, 1H), 7.46-7.39 (m, | |
| | | 4H), 7.19-7.13 (m, 2H), 6.88 (d, J = | |
| | | 7.9 Hz, 1H), 6.58-6.51 (m, 1H), 5.32- | |
| | | 5.24 (m, 2H), 4.33-4.28 (m, 2H), | |
| | | 4.12-4.08 (m, 1H), 3.97-3.85 (m, | |
| | | 4H), 3.36-3.31 (m, 2H), 3.18-3.00 | |
| | | (m, 4H), 2.39-2.35 (m, 1H), 2.26 (s, | |
| | | 3H), 2.21-2.14 (m, 1H), 2.17-2.10 | |
| | | (m, 1H), 2.07-2.03 (m, 1H), 1.69- | |
| | | 1.63 (m, 2H). | |
| 210 | 760.2 | ¹H NMR (500 MHz, Chloroform-d) δ | 4 |
| | | 8.37 (d, J = 2.2 Hz, 1H), 8.28-8.26 | |
| | | (d, J = 2.2 Hz, 1H), 7.90 (d, J = 2.0 | |
| | | Hz, 1H), 7.57-7.51 (m, 1H), 7.46- | |
| | | 7.39 (m, 4H), 7.18-7.11 (m, 2H), 6.88 | |
| | | (d, J = 7.9 Hz, 1H), 6.58-6.51 (m, | |
| | | 1H), 5.32-5.24 (m, 2H), 4.36-4.29 | |
| | | (m, 2H), 4.02-3.93 (m, 3H), 3.87- | |
| | | 3.76 (m, 2H), 3.35-3.31 (m, 2H), 3.03- | |
| | | 2.94 (m, 2H), 2.91-2.84 (m, 5H), | |
| | | 2.25-2.15 (m, 2H). | |
| 211 | 770.2 | ¹H NMR (500 MHz, DMSO-d₆) δ | 4 |
| | | 8.28 (d, J = 2.2 Hz, 1H), 8.13 (d, J = | |
| | | 2.2 Hz, 1H), 7.96-7.92 (m, 1H), 7.62 | |
| | | (d, J = 2.2 Hz, 1H), 7.52-7.49 (m, | |
| | | 1H), 7.47-7.43 (m, 3H), 7.29- | |
| | | 7.26 (m, 2H), 1H), 6.85-6.83 (m, | |
| | | 1H), 6.47-6.45 (m, 1H), 5.34-5.31 | |
| | | (m, 2H), 4.40-4.25 (m, 2H), 4.04- | |
| | | 3.70 (m, 5H), 3.34-3.27 (m, 2H), | |
| | | 3.04-2.82 (m, 4H), 2.30-2.06 (m, | |
| | | 4H), 1.72-1.59 (m, 2H). | |
| 212 | 746.2 | ¹H NMR (500 MHz, Chloroform-d) δ | 4 |
| | | 8.32 (d, J = 1.7 Hz, 1H), 8.30 (d, J = | |
| | | 2.2 Hz, 1H), 7.99-7.96 (m, 1H), | |
| | | 7.84 (t, J = 2.0 Hz, 1H), 7.58-7.52 | |
| | | (m, 1H), 7.46-7.39 (m, 4H), 7.28- | |
| | | 7.21 (m, 2H), 6.88 (d, J = 7.9 Hz, | |
| | | 1H), 6.58-6.51 (m, 1H), 5.32-5.24 | |
| | | (m, 2H), 4.32-4.28 (m, 2H), 4.12- | |
| | | 4.08 (m, 1H), 3.97-3.85 (m, 4H), | |
| | | 3.35-3.28 (m, 2H), 3.18-3.00 (m, | |
| | | 4H), 2.39-2.35 (m, 1H), 2.06-2.01 | |
| | | (m, 1H). | |
| 213 | 743.2 | ¹H NMR (500 MHz, Chloroform-d) δ | 4 |
| | | 8.37 (d, J = 2.2 Hz, 1H), 8.28-8.26 | |
| | | (d, J = 2.2 Hz, 1H), 7.95 (d, J = 2.0 | |
| | | Hz, 1H), 7.49-7.39 (m, 5H), 7.18- | |
| | | 7.11 (m, 2H), 6.88 (d, J = 7.9 Hz, | |
| | | 1H), 6.58-6.51 (m, 1H), 5.32-5.24 | |
| | | (m, 2H), 4.36-4.29 (m, 2H), 4.02- | |
| | | 3.93 (m, 3H), 3.87-3.76 (m, 2H), | |
| | | 3.35-3.31 (m, 2H), 3.03-2.94 (m, | |
| | | 2H), 2.91-2.84 (m, 5H), 2.25-2.15 | |
| | | (m, 2H). | |
| 214 | 726.2 | ¹H NMR (500 MHz, Chloroform-d) δ | 4 |
| | | 11.79 (s, 1H), 10.71 (s, 1H), 8.30 (d, | |
| | | J = 2.0 Hz, 1H), 8.04-8.02 (m, 1H), | |
| | | 7.90 (d, J = 2.0 Hz, 1H), 7.57-7.51 | |
| | | (m, 1H), 7.46-7.39 (m, 4H), 7.19- | |
| | | 7.13 (m, 2H), 6.88 (d, J = 7.9 Hz, | |
| | | 1H), 6.58-6.51 (m, 1H), 5.32-5.24 | |
| | | (m, 2H), 4.36-4.29 (m, 2H), 4.02- | |
| | | 3.93 (m, 3H), 3.87-3.76 (m, 2H), | |
| | | 3.35-3.30 (m, 2H), 3.03-2.94 (m, | |
| | | 2H), 2.95-2.84 (m, 5H), 2.25-2.15 | |
| | | (m, 2H). | |
| 215 | 746.2 | ¹H NMR (500 MHz, Chloroform-d) δ | 4 |
| | | 8.49 (d, J = 1.4 Hz, 1H), 7.90 (d, J = | |
| | | 2.0 Hz, 1H), 7.90 (d, J = 7.9 Hz, | |
| | | 1H), 7.75 (d, J = 7.9 Hz, 1H), 7.57- | |
| | | 7.51 (m, 1H), 7.46-7.39 (m, 4H), | |
| | | 7.19-7.13 (m, 2H), 6.88 (d, J = 7.9 | |
| | | Hz, 1H), 6.58-6.51 (m, 1H), 5.32- | |
| | | 5.24 (m, 2H), 4.36-4.29 (m, 2H), | |
| | | 4.02-3.93 (m, 3H), 3.87-3.76 (m, | |
| | | 2H), 3.35-3.31 (m, 2H), 3.03-2.94 | |
| | | (m, 2H), 2.88-2.85 (m, 2H), 2.25- | |
| | | 2.15 (m, 2H). | |
| 216 | 718.3 | ¹H NMR (500 MHz, Chloroform-d) δ | 4 |
| | | 8.38 (d, J = 1.4 Hz, 1H), 7.90 (d, J = | |
| | | 2.0 Hz, 1H), 7.90 (d, J = 7.8 Hz, 1H), | |
| | | 7.77 (d, J = 7.9 Hz, 1H), 7.57-7.51 | |
| | | (m, 1H), 7.46-7.39 (m, 4H), 7.18- | |
| | | 7.11 (m, 2H), 6.88 (d, J = 7.9 Hz, | |
| | | 1H), 6.58-6.51 (m, 1H), 5.32-5.24 | |
| | | (m, 2H), 4.36-4.29 (m, 2H), 4.02- | |
| | | 3.93 (m, 3H), 3.87-3.76 (m, 2H), | |
| | | 3.35-3.31 (m, 2H), 3.03-2.94 (m, | |
| | | 2H), 2.88-2.85 (m, 2H), 2.34-2.18 | |
| | | (m, 3H), 1.07-0.93 (m, 4H). | |
| 217 | 742.2 | ¹H NMR (500 MHz, Chloroform-d) δ | 4 |
| | | 8.53 (d, J = 2.2 Hz, 1H), 7.93 (d, J = | |
| | | 2.0 Hz, 1H), 7.82 (d, J = 2.2 Hz, 1H), | |
| | | 7.57-7.51 (m, 1H), 7.46-7.39 (m, | |
| | | 4H), 7.18-7.11 (m, 2H), 6.88 (d, J = | |
| | | 7.9 Hz, 1H), 6.58-6.51 (m, 1H), 5.32- | |
| | | 5.24 (m, 2H), 4.36-4.29 (m, 2H), | |
| | | 4.02-3.93 (m, 3H), 3.87-3.76 (m, | |
| | | 2H), 3.35-3.31 (m, 2H), 3.03-2.94 | |
| | | (m, 2H), 2.88-2.85 (m, 2H), 2.68 (s, | |
| | | 3H), 2.25-2.15 (m, 2H). | |
| 218 | 732.3 | ¹H NMR (500 MHz, Chloroform-d) δ | 4 |
| | | 12.40 (s, 1H), 10.28 (s, 1H), 8.34 (d, | |
| | | J = 2.2 Hz, 1H), 7.90 (d, J = 2.0 Hz, | |
| | | 1H), 7.84-7.80 (m, 1H), 7.55 (dd, J = | |
| | | 8.6, 2.0 Hz, 1H), 7.49-7.43 (m, | |
| | | 2H), 7.42-7.34 (m, 2H), 7.17-7.15 | |
| | | (m, 2H), 6.83-6.81 (m, 1H), 6.55- | |
| | | 6.52 (m, 1H), 5.35-5.24 (m, 2H), | |
| | | 4.35 (dd, J = 15.6, 5.2 Hz, 1H), 4.28- | |
| | | 4.25 (m, 1H), 4.06-3.97 (m, 2H), | |
| | | 3.93 (d, J = 13.7 Hz, 1H), 3.85-3.82 | |
| | | (m, 1H), 3.76-3.74 (m, 1H), 3.32- | |
| | | 3.28 (m, 2H), 3.05-3.01 (m, 1H), 3.00- | |
| | | 2.97 (m, 1H), 2.89-2.86 (m, 2H), | |
| | | 2.68 (s, 3H), 2.33-2.24 (m, 2H), | |
| | | 2.24-2.18 (m, 1H), 1.07-0.92 (m, | |
| | | 4H). | |
| 219 | 728.2 | ¹H NMR (500 MHz, Chloroform-d) δ | 4 |
| | | 12.43 (s, 1H), 10.38 (s, 1H), 8.65 (d, | |
| | | J = 1.6 Hz, 1H), 7.90 (d, J = 1.8 Hz, | |
| | | 1H), 7.92 (dd, J = 7.9, 1.8 Hz, 1H), | |
| | | 7.76 (d, J = 7.9 Hz, 1H), 7.55 (dd, J = | |
| | | 8.6, 2.0 Hz, 1H), 7.49-7.43 (m, 2H), | |
| | | 7.42-7.34 (m, 2H), 7.21-7.12 (m, | |
| | | 3H), 6.83-6.81 (m, 1H), 6.55- | |
| | | 6.51 (m, 1H), 5.35-5.24 (m, 2H), | |
| | | 4.35 (dd, J = 15.6, 5.2 Hz, 1H), 4.28- | |
| | | 4.24 (m, 1H), 4.06-3.97 (m, 2H), | |
| | | 3.93 (d, J = 13.7 Hz, 1H), 3.85-3.82 | |
| | | (m, 1H), 3.76-3.74 (m, 1H), 3.32- | |
| | | 3.30 (m, 2H), 3.05-3.03 (m, 1H), | |
| | | 3.00-2.97 (m, 1H), 2.89-2.87 (m, | |
| | | 2H), 2.31-2.15 (m, 2H). | |
| 220 | 742.2 | ¹H NMR (500 MHz, Chloroform-d) δ | 4 |
| | | 12.44 (s, 1H), 10.21 (s, 1H), 8.44- | |
| | | 8.42 (m, 2H), 8.05 (d, J = 2.1 Hz, | |
| | | 2H), 7.90 (d, J = 2.1 Hz, 2H), 7.60- | |
| | | 7.52 (m, 3H), 7.49-7.33 (m, 10H), | |
| | | 7.17-7.15 (m, 4H), 6.86-6.84 (m, | |
| | | 2H), 6.55-6.51 (m, 2H), 5.35-5.24 | |
| | | (m, 4H), 4.35-4.31 (m, 2H), 4.28- | |
| | | 4.21 (m, 2H), 4.06-3.97 (m, 4H), | |
| | | 3.93 (d, J = 13.7 Hz, 2H), 3.85-3.81 | |
| | | (m, 2H), 3.76-3.73 (m, 2H), 3.32- | |
| | | 3.29 (m, 4H), 3.05-3.01 (m, 2H), 3.00- | |
| | | 2.98 (m, 2H), 2.94-2.87 (m, 9H), | |
| | | 2.86-2.81 (m, 1H), 2.30-2.22 (m, | |
| | | 4H). | |
| 221 | 709.2 | ¹H NMR (500 MHz, Chloroform-d) δ | 4 |
| | | 8.46 (d, J = 5.0 Hz, 1H), 7.92 (d, J = | |
| | | 2.0 Hz, 1H), 7.58-7.42 (m, 4H), 7.42- | |
| | | 7.34 (m, 2H), 7.21-7.13 (m, 2H), | |
| | | 6.88-6.84 (m, 2H), 5.35-5.24 (m, | |
| | | 2H), 4.37-4.33 (m, 1H), 4.31-4.24 | |
| | | (m, 1H), 4.06-3.97 (m, 2H), 3.96- | |
| | | 3.70 (m, 3H), 3.36-3.28 (m, 2H), | |
| | | 3.08-2.97 (m, 2H), 2.95 (s, 3H), 2.93- | |
| | | 2.30 (m, 3H), 2.09-1.99 (m, 1H). | |
| 222 | 723.3 | ¹H NMR (500 MHz, Chloroform-d) δ | 4 |
| | | 7.92 (d, J = 2.0 Hz, 1H), 7.78 (d, J = | |
| | | 5.2 Hz, 1H), 7.57-7.55 (s, 1H), 7.49- | |
| | | 7.45 (m, 2H), 7.42-7.13 (m, 4H), | |
| | | 6.88-6.84 (m, 2H), 5.35-5.24 (m, | |
| | | 2H), 4.35-4.27 (m, 2H), 4.06-3.97 | |
| | | (m, 2H), 3.96-3.70 (m, 3H), 3.36- | |
| | | 2.97 (m, 4H), 2.93-2.84 (m, 2H), | |
| | | 2.65 (s, 3H), 2.52 (s, 3H), 2.36-2.31 | |
| | | (m, 1H), 2.09-1.99 (m, 1H). | |
| 223 | 719.3 | ¹H NMR (500 MHz, Chloroform-d) δ | 4 |
| | | 8.58 (d, J = 1.8 Hz, 1H), 7.90 (d, J = | |
| | | 2.1 Hz, 1H), 7.92-7.91 (m, 2H), | |
| | | 7.55-7.42 (m, 2H), 7.42-7.34 (m, | |
| | | 3H), 7.21-7.13 (m, 2H), 6.89-6.83 | |
| | | (m, 2H), 5.35-5.24 (m, 2H), 4.35- | |
| | | 4.24 (m, 2H), 4.06-3.97 (m, 2H), | |
| | | 3.97-3.70 (m, 3H), 3.36-3.28 (m, | |
| | | 2H), 3.12-2.97 (m, 3H), 2.93-2.84 | |
| | | (m, 2H), 2.09-1.99 (m, 2H), 1.65 (d, | |
| | | J = 6.7 Hz, 3H), 1.61 (s, 3H). | |
| 224 | 709.2 | ¹H NMR (500 MHz, Chloroform-d) δ | 4 |
| | | 7.95-7.89 (m, 2H), 7.57-7.55 (s, | |
| | | 1H), 7.49-7.45 (m, 3H), 7.42-7.13 | |
| | | (m, 4H), 6.88-6.84 (m, 3H), 5.35- | |
| | | 5.24 (m, 2H), 4.35-4.27 (m, 2H), | |
| | | 4.06-3.70 (m, 5H), 3.36-3.28 (m, | |
| | | 2H), 3.08-2.97 (m, 2H), 2.93-2.84 | |
| | | (m, 2H), 2.58 (s, 3H), 2.36-2.31 (m, | |
| | | 1H), 2.09-1.99 (m, 1H). | |
| 225 | 700.3 | ¹H NMR (500 MHz, Chloroform-d) δ | 4 |
| | | 8.25 (d, J = 2.2 Hz, 1H), 7.90 (d, J = | |
| | | 2.1 Hz, 1H), 7.76-7.73 (m, 1H), | |
| | | 7.53-7.42 (m, 5H), 7.40-7.33 (m, | |
| | | 2H), 6.88-6.84 (m, 2H), 5.35-5.24 | |
| | | (m, 2H), 4.37-4.33 (m, 1H), 4.31- | |
| | | 4.24 (m, 1H), 4.06-3.97 (m, 2H), | |
| | | 3.96-3.70 (m, 3H), 3.36-3.28 (m, | |
| | | 2H), 3.08-2.97 (m, 2H), 2.96 (s, | |
| | | 3H), 2.91-2.86 (m, 2H), 2.36-2.31 | |
| | | (m, 1H), 2.09-1.99 (m, 1H). | |
| 226 | 723.3 | ¹H NMR (500 MHz, Chloroform-d) δ | 4 |
| | | 8.82 (s, 1H), 7.96 (m, 2H), 7.85 (d, J = | |
| | | 2.2 Hz, 1H), 7.55-7.44 (m, 5H), | |
| | | 7.40-7.33 (m, 2H), 6.89-6.84 (m, | |
| | | 1H), 6.55 (s, 1H), 5.35-5.24 (m, | |
| | | 2H), 4.35-4.27 (m, 2H), 4.06-3.98 | |
| | | (m, 1H), 3.93-3.81 (m, 2H), 3.81- | |
| | | 3.73 (m, 2H), 3.36-3.28 (m, 2H), | |
| | | 3.08-2.97 (m, 2H), 2.93-2.84 (m, | |
| | | 2H), 2.26 (s, 3H), 2.32-2.14 (m, | |
| | | 3H), 2.17-2.10 (m, 1H), 1.71-1.62 | |
| | | (m, 2H). | |
| 227 | 727.3 | ¹H NMR (500 MHz, Chloroform-d) δ | 4 |
| | | 8.28 (s, 1H), 7.95 (m, 2H), 7.82- | |
| | | 7.77 (m, 1H), 7.58-7.44 (m, 5H), | |
| | | 7.40-7.33 (m, 2H), 6.88-6.84 (m, | |
| | | 2H), 5.35-5.24 (m, 2H), 4.39-4.24 | |
| | | (m, 2H), 4.06-3.98 (m, 1H), 3.93 (d, | |
| | | J = 13.9 Hz, 1H), 3.89-3.73 (m, | |
| | | 3H), 3.36-3.28 (m, 2H), 3.08-2.84 | |
| | | (m, 4H), 2.32-2.11 (m, 4H), 1.73- | |
| | | 1.64 (m, 2H). | |
| 228 | 732.3 | ¹H NMR (500 MHz, Chloroform-d) δ | 4 |
| | | 8.53 (d, J = 2.1 Hz, 1H), 7.90 (d, J = | |
| | | 2.0 Hz, 1H), 7.84-7.52 (m, 2H), 7.50- | |
| | | 7.44 (m, 4H), 7.40-7.33 (m, 2H), | |
| | | 6.88-6.84 (m, 2H), 5.35-5.24 (m, | |
| | | 2H), 4.35-4.27 (m, 2H), 4.06-3.70 | |
| | | (m, 5H), 3.36-3.28 (m, 2H), 3.08- | |
| | | 2.84 (m, 4H), 2.66 (s, 3H), 2.36- | |
| | | 2.31 (m, 1H), 2.09-1.99 (m, 1H). | |
| 229 | 696.3 | ¹H NMR (500 MHz, Chloroform-d) δ | 4 |
| | | 8.34 (d, J = 2.2 Hz, 1H), 7.90 (d, J = | |
| | | 2.1 Hz, 1H), 7.84-7.80 (m, 1H), | |
| | | 7.55-7.41 (m, 5H), 7.40-7.33 (m, | |
| | | 2H), 6.88-6.84 (m, 2H), 5.35-5.24 | |
| | | (m, 2H), 4.37-4.33 (m, 1H), 4.33- | |
| | | 3.97 (m, 3H), 3.96-3.83 (m, 2H), | |
| | | 3.78-3.74 (m, 1H), 3.36-3.28 (m, | |
| | | 2H), 3.10-2.97 (m, 2H), 2.97 (s, | |
| | | 3H), 2.93-2.84 (m, 2H), 2.68 (s, | |
| | | 3H), 2.36-2.31 (m, 1H), 2.09-1.99 | |
| | | (m, 1H). | |
| 230 | 709.3 | ¹H NMR (500 MHz, Chloroform-d) δ | 4 |
| | | 9.09 (s, 1H), 8.08 (d, J = 2.0 Hz, 1H), | |
| | | 7.90 (d, J = 2.2 Hz, 1H), 7.19 (m, | |
| | | 1H), 7.53-7.42 (m, 5H), 7.40-7.33 | |
| | | (m, 2H), 7.15 (d, J = 7.7 Hz, 1H), | |
| | | 6.88-6.84 (m, 2H), 5.35-5.24 (m, | |
| | | 2H), 4.38-4.26 (m, 2H), 4.06-3.98 | |
| | | (m, 1H), 3.93 (d, J = 13.9 Hz, 1H), | |
| | | 3.90-3.70 (m, 3H), 3.36-3.28 (m, | |
| | | 2H), 3.08-2.97 (m, 2H), 2.93-2.84 | |
| | | (m, 2H), 2.34-2.15 (m, 3H), 2.18- | |
| | | 2.11 (m, 1H), 1.70-1.65 (m, 2H). | |
| 231 | 696.3 | ¹H NMR (500 MHz, Chloroform-d) δ | 4 |
| | | 8.46 (d, J = 1.4 Hz, 1H), 7.90 (d, J = | |
| | | 2.1 Hz, 1H), 7.94-7.73 (m, 2H), | |
| | | 7.53-7.42 (m, 5H), 7.40-7.33 (m, | |
| | | 2H), 6.88-6.84 (m, 2H), 5.35-5.24 | |
| | | (m, 2H), 4.37-4.33 (m, 1H), 4.33- | |
| | | 3.97 (m, 3H), 3.96-3.70 (m, 3H), | |
| | | 3.36-3.28 (m, 2H), 3.08-3.02 (m, | |
| | | 1H), 3.02-2.97 (m, 1H), 2.93-2.84 | |
| | | (m, 2H), 2.83-2.75 (m, 2H), 2.40- | |
| | | 2.30 (m, 1H), 2.09-1.99 (m, 1H), | |
| | | 1.38 (d, J = 10.4 Hz, 3H). | |
| 232 | 736.3 | ¹H NMR (500 MHz, Chloroform-d) δ | 4 |
| | | 7.90 (d, J = 1.8 Hz, 1H), 7.92-7.91 | |
| | | (m, 1H), 7.79 (d, J = 7.9 Hz, 1H), | |
| | | 7.55 (s, 1H), 7.50-7.44 (m, 4H), | |
| | | 7.40-7.33 (m, 2H), 6.88-6.84 (m, | |
| | | 2H), 5.35-5.24 (m, 2H), 4.37-4.33 | |
| | | (m, 1H), 4.33-3.97 (m, 3H), 3.96- | |
| | | 3.70 (m, 3H), 3.36-3.02 (m, 3H), | |
| | | 3.02-2.97 (m, 1H), 2.93-2.84 (m, | |
| | | 2H), 2.36-2.31 (m, 1H), 2.09-1.99 | |
| | | (m, 1H). | |
| 233 | 722.3 | ¹H NMR (500 MHz, Chloroform-d) δ | 4 |
| | | 8.23 (d, J = 2.2 Hz, 1H), 7.90 (d, J = | |
| | | 2.0 Hz, 1H), 7.84-7.80 (m, 1H), | |
| | | 7.53-7.42 (m, 5H), 7.40-7.33 (m, | |
| | | 2H), 6.88-6.84 (m, 2H), 5.35-5.24 | |
| | | (m, 2H), 4.41-4.39 (m, 1H), 4.33- | |
| | | 3.97 (m, 3H), 3.96-3.70 (m, 3H), | |
| | | 3.36-3.02 (m, 3H), 3.02-2.97 (m, | |
| | | 1H), 2.93-2.84 (m, 2H), 2.68 (s, | |
| | | 3H), 2.39-2.27 (m, 2H), 2.09-1.99 | |
| | | (m, 1H), 1.07-0.92 (m, 4H). | |
| 234 | 692.2 | ¹H NMR (500 MHz, DMSO-d₆) δ | 4 |
| | | 12.35 (s, 1H), 10.43 (s, 1H), 8.89 (d, | |
| | | J = 1.7 Hz, 1H), 8.61 (s, 1H), 7.97 (d, | |
| | | J = 2.2 Hz, 1H), 7.59-7.57 (m, 1H), | |
| | | 7.53-7.49 (m, 3H), 7.43-7.32 (m, | |
| | | 1H), 7.35-7.26 (m, 2H), 6.86-6.69 | |
| | | (m, 2H), 5.33-5.29 (m, 2H), 4.37- | |
| | | 4.33 (m, 1H), 4.32-4.28 (m, 1H), | |
| | | 4.05-3.98 (m, 1H), 3.89-3.68 (m, | |
| | | 4H), 3.35-2.94 (m, 4H), 2.91-2.82 | |
| | | (m, 2H), 2.48 (s, 3H), 2.19-2.10 (m, | |
| | | 2H). | |
| 235 | 682.3 | ¹H NMR (500 MHz, DMSO-d₆) δ | 4 |
| | | 8.42 (d, J = 1.8 Hz, 1H), 7.97 (d, J = | |
| | | 2.1 Hz, 1H), 7.91-7.90 (m, 1H), | |
| | | 7.70 (d, J = 8.4 Hz, 1H), 7.63-7.50 | |
| | | (m, 3H), 7.52-7.44 (m, 3H), 7.44- | |
| | | 7.40 (m, 1H), 6.86-6.69 (m, 2H), | |
| | | 5.34-5.31 (m, 2H), 4.37-4.33 (m, | |
| | | 1H), 4.32-4.28 (m, 1H), 4.05-3.95 | |
| | | (m, 2H), 3.95-3.82 (m, 2H), 3.75- | |
| | | 3.69 (m, 1H), 3.35-2.94 (m, 4H), | |
| | | 2.91-2.82 (m, 2H), 2.43 (s, 3H), | |
| | | 2.38-2.33 (m, 1H), 2.09-2.02 (m, | |
| | | 1H). | |
| 236 | 682.3 | ¹H NMR (500 MHz, DMSO-d₆) δ | 4 |
| | | 8.55-8.53 (m, 1H), 8.14 (d, J = 1.7 | |
| | | Hz, 1H), 7.97 (d, J = 2.1 Hz, 1H), | |
| | | 7.88 (d, J = 2.1 Hz, 1H), 7.63-7.50 | |
| | | (m, 3H), 7.52-7.44 (m, 3H), 7.42- | |
| | | 7.40 (m, 1H), 6.83-6.81 (m, 1H), 6.47- | |
| | | 6.45 (m, 1H), 5.34-5.31 (m, 2H), | |
| | | 4.37-4.33 (m, 1H), 4.32-4.28 (m, | |
| | | 1H), 4.05-3.82 (m, 4H), 3.75- | |
| | | 3.69 (m, 1H), 3.35-2.94 (m, 4H), | |
| | | 2.91-2.82 (m, 2H), 2.61 (s, 3H), | |
| | | 2.38-2.33 (m, 1H), 2.09-2.02 (m, | |
| | | 1H). | |
| 237 | 692.2 | ¹H NMR (500 MHz, DMSO-d₆) δ | 4 |
| | | 12.12 (s, 1H), 10.97 (s, 1H), 9.07 (s, | |
| | | 1H), 8.30 (s, 1H), 7.95 (d, J = 2.2 Hz, | |
| | | 1H), 7.59-7.57 (m, 1H), 7.53-7.41 | |
| | | (m, 4H), 7.35-7.29 (m, 2H), 6.86- | |
| | | 6.69 (m, 2H), 5.34-5.31 (m, 2H), 4.37- | |
| | | 4.33 (m, 1H), 4.32-4.28 (m, 1H), | |
| | | 4.05-3.98 (m, 1H), 3.89-3.68 (m, | |
| | | 4H), 3.35-2.94 (m, 4H), 2.91-2.82 | |
| | | (m, 2H), 2.42 (s, 3H), 2.29-2.17 (m, | |
| | | 2H). | |
| 238 | 693.2 | ¹H NMR (500 MHz, DMSO-d₆) δ | 4 |
| | | 7.94 (d, J = 2.2 Hz, 1H), 7.61-7.55 | |
| | | (s, 1H), 7.53-7.49 (m, 3H), 7.43- | |
| | | 7.32 (m, 1H), 7.35-7.26 (m, 2H), | |
| | | 6.86-6.69 (m, 2H), 5.34-5.31 (m, | |
| | | 2H), 4.37-4.33 (m, 1H), 4.32-4.28 | |
| | | (m, 1H), 4.05-3.98 (m, 1H), 3.77- | |
| | | 3.68 (m, 4H), 3.35-2.94 (m, 4H), | |
| | | 2.91-2.82 (m, 2H), 2.50 (s, 3H), | |
| | | 2.19-2.10 (m, 2H). | |
| 239 | 692.2 | ¹H NMR (500 MHz, DMSO-d₆) δ | 4 |
| | | 8.06 (d, J = 7.9 Hz, 1H), 8.08 (d, J = | |
| | | 7.6 Hz, 1H), 7.95 (d, J = 2.2 Hz, 1H), | |
| | | 7.59-7.57 (m, 1H), 7.53-7.52 (m, | |
| | | 1H), 7.49-7.45 (m, 2H), 7.46-7.40 | |
| | | (m, 1H), 7.35-7.26 (m, 2H), 6.86- | |
| | | 6.69 (m, 2H), 5.34-5.31 (m, 2H), 4.37- | |
| | | 4.33 (m, 1H), 4.32-4.28 (m, 1H), | |
| | | 4.05-3.98 (m, 1H), 3.77-3.68 (m, | |
| | | 4H), 3.35-2.94 (m, 4H), 2.91-2.82 | |
| | | (m, 2H), 2.52 (s, 3H), 2.19-2.10 (m, | |
| | | 2H). | |
| 240 | 693.2 | ¹H NMR (500 MHz, DMSO-d₆) δ | 4 |
| | | 12.38 (s, 1H), 10.65 (s, 1H), 9.02 (s, | |
| | | 1H), 7.94 (d, J = 2.2 Hz, 1H), 7.61- | |
| | | 7.55 (s, 1H), 7.53-7.49 (m, 3H), | |
| | | 7.43-7.32 (m, 1H), 7.35-7.26 (m, | |
| | | 2H), 6.86-6.69 (m, 2H), 5.34-5.31 | |
| | | (m, 2H), 4.37-4.33 (m, 1H), 4.32- | |
| | | 4.28 (m, 1H), 4.05-3.98 (m, 1H), | |
| | | 3.77-3.68 (m, 4H), 3.35-2.94 (m, | |
| | | 4H), 2.91-2.82 (m, 2H), 2.51 (s, | |
| | | 3H), 2.19-2.10 (m, 2H). | |
| 241 | 678.2 | ¹H NMR (500 MHz, DMSO-d₆) δ | 4 |
| | | 10.56 (s, 1H), 8.33 (t, J = 1.2 Hz, | |
| | | 1H), 8.00-7.95 (m, 3H), 7.55-7.53 | |
| | | (m, 1H), 7.53-7.49 (m, 3H), | |
| | | 7.43-7.32 (m, 1H), 7.35-7.26 (m, | |
| | | 2H), 6.86-6.69 (m, 2H), 5.34-5.31 | |
| | | (m, 2H), 4.37-4.33 (m, 1H), 4.32- | |
| | | 4.28 (m, 1H), 4.05-3.98 (m, 1H), | |
| | | 3.77-3.68 (m, 4H), 3.35-2.94 (m, | |
| | | 4H), 2.91-2.82 (m, 2H), 2.19-2.10 | |
| | | (m, 2H). | |
| 242 | 692.2 | ¹H NMR (500 MHz, DMSO-d₆) δ | 4 |
| | | 8.15 (d, J = 2.2 Hz, 1H), 8.00-7.95 | |
| | | (m, 2H), 7.55-7.53 (m, 1H), 7.53- | |
| | | 7.49 (m, 3H), 7.43-7.32 (m, 1H), | |
| | | 7.35-7.26 (m, 2H), 6.86-6.69 (m, | |
| | | 2H), 5.34-5.31 (m, 2H), 4.37-4.33 | |
| | | (m, 1H), 4.32-4.28 (m, 1H), 4.05- | |
| | | 3.98 (m, 1H), 3.77-3.68 (m, 4H), | |
| | | 3.35-2.94 (m, 4H), 2.91-2.82 (m, | |
| | | 2H), 2.70 (s, 3H), 2.19-2.10 (m, | |
| | | 2H). | |
| 243 | 731.3 | ¹H NMR (500 MHz, DMSO-d₆) δ | 4 |
| | | 8.42 (d, J = 1.4 Hz, 1H), 8.08 (d, J = | |
| | | 2.1 Hz, 1H), 7.92 (dd, J = 7.8, 1.7 | |
| | | Hz, 1H), 7.65 (d, J = 7.9 Hz, 1H), | |
| | | 7.59-7.53 (m, 1H), 7.52-7.45 (m, | |
| | | 2H), 7.37-7.34 (m, 1H), 7.32-7.28 | |
| | | (m, 1H), 7.25-7.19 (m, 1H), 6.57- | |
| | | 6.53 (m, 1H), 6.23-6.19 (m, 1H), | |
| | | 5.38-5.33 (m, 1H), 5.36-5.28 (m, | |
| | | 3H), 4.44-4.36 (m, 1H), 4.33-4.29 | |
| | | (m, 1H), 3.91-3.82 (m, 2H), 3.75- | |
| | | 3.69 (m, 3H), 3.64-3.56 (m, 2H), | |
| | | 3.20 (d, J = 8.0 Hz, 1H), 2.38-2.33 | |
| | | (m, 4H), 2.12-2.00 (m, 2H), 1.99- | |
| | | 1.89 (m, 3H), 1.84-1.76 (m, 2H). | |
| 244 | 723.3 | ¹H NMR (500 MHz, DMSO-d₆) δ | 4 |
| | | 8.24 (d, J = 2.2 Hz, 1H), 8.03 (d, J = | |
| | | 1.6 Hz, 1H), 7.80-7.77 (m, 1H), | |
| | | 7.59-7.57 (m, 1H), 7.52-7.45 (m, | |
| | | 2H), 7.37-7.34 (m, 1H), 7.32-7.28 | |
| | | (m, 1H), 7.25-7.19 (m, 1H), 6.57- | |
| | | 6.53 (m, 1H), 6.23-6.19 (m, 1H), | |
| | | 5.35-5.34 (m, 2H), 4.33-4.29 (m, | |
| | | 1H), 4.32-4.26 (m, 1H), 3.93-3.84 | |
| | | (m, 2H), 3.76-3.68 (m, 3H), 3.64- | |
| | | 3.56 (m, 2H), 3.20 (d, J = 8.0 Hz, | |
| | | 1H), 2.42 (s, 3H), 2.32-2.24 (m, | |
| | | 1H), 2.20-2.14 (m, 1H), 2.12-2.05 | |
| | | (m, 1H), 1.99-1.89 (m, 3H), 1.84- | |
| | | 1.76 (m, 2H). | |
| 245 | 719.3 | ¹H NMR (500 MHz, DMSO-d₆) δ | 4 |
| | | 12.05 (s, 1H), 10.26 (s, 1H), 8.32 (d, | |
| | | J = 2.2 Hz, 1H), 8.08 (d, J = 2.1 Hz, | |
| | | 1H), 7.80 (d, J = 2.2 Hz, 1H), 7.55- | |
| | | 7.53 (m, 1H), 7.52-7.45 (m, 2H), | |
| | | 7.37-7.34 (m, 1H), 7.32-7.28 (m, | |
| | | 1H), 7.25-7.19 (m, 1H), 6.57-6.53 | |
| | | (m, 1H), 6.23-6.19 (m, 1H), 5.35- | |
| | | 5.34 (m, 2H), 4.33-4.29 (m, 1H), | |
| | | 4.30-4.26 (m, 1H), 3.93-3.84 (m, | |
| | | 2H), 3.76-3.68 (m, 3H), 3.64-3.56 | |
| | | (m, 2H), 3.21-3.18 (m, 1H), 2.72 (s, | |
| | | 3H), 2.45 (s, 3H), 2.32-2.24 (m, | |
| | | 1H), 2.20-2.14 (m, 1H), 2.12-2.05 | |
| | | (m, 1H), 1.99-1.89 (m, 3H), 1.84- | |
| | | 1.76 (m, 2H). | |
| 246 | 705.3 | ¹H NMR (500 MHz, DMSO-d₆) δ | 4 |
| | | 8.42 (d, J = 1.8 Hz, 1H), 8.08 (d, J = | |
| | | 2.0 Hz, 1H), 7.91-7.90 (m, 1H), | |
| | | 7.70 (d, J = 7.9 Hz, 1H), 7.56 (dd, J = | |
| | | 8.7, 1.8 Hz, 1H), 7.52-7.45 (m, 2H), | |
| | | 7.37-7.34 (m, 1H), 7.32-7.28 (m, | |
| | | 1H), 7.25-7.19 (m, 1H), 6.57-6.53 | |
| | | (m, 1H), 6.23-6.19 (m, 1H), 5.35- | |
| | | 5.34 (m, 2H), 4.44-4.36 (m, 1H), | |
| | | 4.33-4.29 (m, 1H), 3.93-3.82 (m, | |
| | | 2H), 3.75-3.69 (m, 3H), 3.64-3.56 | |
| | | (m, 2H), 3.21-3.18 (m, 1H), 2.43 (s, | |
| | | 3H), 2.38-2.33 (m, 1H), 2.12-2.00 | |
| | | (m, 2H), 1.99-1.89 (m, 3H), 1.84- | |
| | | 1.76 (m, 2H). | |
| 247 | 746.3 | ¹H NMR (500 MHz, DMSO-d₆) δ | 4 |
| | | 10.23 (s, 1H), 10.12 (s, 1H), 8.08 (d, | |
| | | J = 2.2 Hz, 1H), 7.95 (d, J = 2.2 Hz, | |
| | | 1H), 7.88 (d, J = 2.1 Hz, 1H), 7.55- | |
| | | 7.53 (m, 1H), 7.52-7.45 (m, 2H), | |
| | | 7.37-7.34 (m, 1H), 7.32-7.28 (m, | |
| | | 1H), 7.25-7.19 (m, 1H), 6.57-6.53 | |
| | | (m, 1H), 6.23-6.19 (m, 1H), 5.35- | |
| | | 5.34 (m, 2H), 4.44-4.36 (m, 1H), | |
| | | 4.33-4.29 (m, 1H), 3.93-3.79 (m, | |
| | | 2H), 3.78-3.68 (m, 3H), 3.64-3.56 | |
| | | (m, 2H), 3.21-3.18 (m, 1H), 2.21 (s, | |
| | | 3H), 2.30-2.05 (m, 5H), 1.99-1.89 | |
| | | (m, 3H), 1.84-1.76 (m, 2H), 1.72- | |
| | | 1.61 (m, 2H). | |
| 248 | 739.2 | ¹H NMR (500 MHz, DMSO-d₆) δ | 4 |
| | | 8.30 (d, J = 2.2 Hz, 1H), 8.04-7.99 | |
| | | (m, 2H), 7.55-7.53 (m, 1H), 7.52- | |
| | | 7.45 (m, 2H), 7.37-7.34 (m, 1H) | |
| | | 7.32-7.28 (m, 1H), 7.25-7.19 (m, | |
| | | 1H), 6.57-6.53 (m, 1H), 6.23-6.19 | |
| | | (m, 1H), 5.35-5.34 (m, 2H), 4.33- | |
| | | 4.29 (m, 1H), 4.32-4.26 (m, 1H), | |
| | | 3.93-3.84 (m, 2H), 3.76-3.68 (m, | |
| | | 3H), 3.64-3.56 (m, 2H), 3.21-3.18 | |
| | | (m, 1H), 2.45 (s, 3H), 2.32-2.24 (m, | |
| | | 1H), 2.20-2.14 (m, 1H), 2.12-2.05 | |
| | | (m, 1H), 1.99-1.89 (m, 3H), 1.84- | |
| | | 1.76 (m, 2H). | |
| 249 | 721.3 | ¹H NMR (500 MHz, DMSO-d₆) δ | 4 |
| | | 10.66 (s, 1H), 10.53 (s, 1H), 10.04 (s, | |
| | | 1H), 8.25 (d, J = 2.2 Hz, 1H), 8.08 (d, | |
| | | J = 2.2 Hz, 1H), 7.83 (d, J = 2.2 Hz, | |
| | | 1H), 7.55-7.53 (m, 1H), 7.52-7.45 | |
| | | (m, 2H), 7.37-7.34 (m, 1H), 7.32- | |
| | | 7.28 (m, 1H), 7.25-7.19 (m, 1H), | |
| | | 6.57-6.53 (m, 1H), 6.23-6.19 (m, | |
| | | 1H), 5.35-5.34 (m, 2H), 4.44-4.36 | |
| | | (m, 1H), 4.33-4.29 (m, 1H), 3.93- | |
| | | 3.79 (m, 2H), 3.78-3.68 (m, 3H), | |
| | | 3.64-3.56 (m, 2H), 3.20 (d, J = 8.0 | |
| | | Hz, 1H), 2.30 (s, 3H), 2.29-2.17 (m, | |
| | | 2H), 2.12-2.05 (m, 1H), 1.99-1.93 | |
| | | (m, 1H), 1.96-1.89 (m, 2H), 1.84- | |
| | | 1.76 (m, 2H). | |
| 250 | 719.3 | ¹H NMR (500 MHz, DMSO-d₆) δ | 4 |
| | | 8.43 (d, J = 2.1 Hz, 1H), 8.08 (d, J = | |
| | | 2.1 Hz, 1H), 7.92-7.91 (m, 1H), | |
| | | 7.73 (d, J = 7.9 Hz, 1H), 7.55-7.53 | |
| | | (m, 1H), 7.52-7.45 (m, 2H), 7.37- | |
| | | 7.34 (m, 1H), 7.32-7.28 (m, 1H), | |
| | | 7.25-7.19 (m, 1H), 6.57-6.53 (m, | |
| | | 1H), 6.23-6.19 (m, 1H), 5.35-5.34 | |
| | | (m, 2H), 4.44-4.36 (m, 1H), 4.33 | |
| | | 4.29 (m, 1H), 3.93-3.82 (m, 2H), | |
| | | 3.75-3.69 (m, 3H), 3.64-3.56 (m, | |
| | | 2H), 3.21-3.18 (m, 1H), 2.87-2.76 | |
| | | (m, 2H), 2.38-2.33 (m, 1H), 2.12- | |
| | | 2.00 (m, 2H), 1.99-1.89 (m, 3H), | |
| | | 1.84-1.76 (m, 2H), 1.33 (t, J = 7.6 | |
| | | Hz, 3H). | |
| 251 | 732.3 | ¹H NMR (500 MHz, DMSO-d₆) δ | 4 |
| | | 12.49 (s, 1H), 10.11 (s, 1H), 8.53 (d, | |
| | | J = 1.4 Hz, 1H), 8.08 (d, J = 2.1 Hz, | |
| | | 1H), 7.92-7.91 (m, 1H), 7.77 (d, J = | |
| | | 7.9 Hz, 1H), 7.55-7.53 (m, 1H), | |
| | | 7.52-7.45 (m, 3H), 7.35-7.26 (m, | |
| | | 2H), 7.15-7.09 (m, 1H), 6.80-6.77 | |
| | | (m, 1H), 5.35-5.34 (m, 2H), 4.44- | |
| | | 4.36 (m, 1H), 4.33-4.29 (m, 1H), | |
| | | 3.93-3.82 (m, 2H), 3.76-3.69 (m, | |
| | | 1H), 3.22 (t, J = 4.4 Hz, 1H), 3.17- | |
| | | 3.07 (m, 1H), 2.99-2.90 (m, 1H), | |
| | | 2.38-2.33 (m, 1H), 2.10-1.88 (m, | |
| | | 5H), 1.83-1.70 (m, 4H), 1.65-1.57 | |
| | | (m, 8H). | |
| 252 | 730.3 | ¹H NMR (500 MHz, DMSO-d₆) δ | 4 |
| | | 8.22 (d, J = 1.4 Hz, 1H), 8.08 (d, J = | |
| | | 2.1 Hz, 1H), 7.92-7.91 (m, 1H) | |
| | | 7.77 (d, J = 7.9 Hz, 1H), 7.55-7.53 | |
| | | (m, 1H), 7.52-7.45 (m, 3H), 7.35- | |
| | | 7.26 (m, 2H), 7.15-7.09 (m, 1H), | |
| | | 6.80-6.77 (m, 1H), 5.35-5.34 (m, | |
| | | 2H), 4.44-4.36 (m, 1H), 4.33-4.29 | |
| | | (m, 1H), 3.93-3.82 (m, 2H), 3.75- | |
| | | 3.69 (m, 1H), 3.22 (t, J = 4.4 Hz, 1H), | |
| | | 2.97-2.90 (m, 1H), 2.38-2.23 (m, | |
| | | 2H), 2.10-1.88 (m, 5H), 1.83-1.70 | |
| | | (m, 4H), 1.62-1.54 (m, 2H), 1.03- | |
| | | 0.89 (m, 4H). | |
| 253 | 774.3 | ¹H NMR (500 MHz, DMSO-d₆) δ | 4 |
| | | 12.39 (s, 1H), 10.59 (s, 1H), 8.38- | |
| | | 8.37 (m, 1H), 8.27 (d, J = 2.0 Hz, | |
| | | 1H), 8.08 (d, J = 2.2 Hz, 1H), 7.55- | |
| | | 7.53 (m, 1H), 7.52-7.45 (m, 2H), | |
| | | 7.37-7.34 (m, 1H), 7.32-7.28 (m, | |
| | | 1H), 7.25-7.19 (m, 1H), 6.57-6.53 | |
| | | (m, 1H), 6.23-6.19 (m, 1H), 5.35- | |
| | | 5.34 (m, 2H), 4.44-4.36 (m, 1H), | |
| | | 4.33-4.29 (m, 1H), 3.93-3.79 (m, | |
| | | 2H), 3.78-3.68 (m, 3H), 3.64-3.56 | |
| | | (m, 2H), 3.21-3.18 (m, 1H), 2.43 (s, | |
| | | 3H), 2.19-2.10 (m, 2H), 2.12-2.05 | |
| | | (m, 1H), 1.99-1.89 (m, 3H), 1.84- | |
| | | 1.76 (m, 2H). | |
| 254 | 751.3 | ¹H NMR (500 MHz, Chloroform-d) δ | |
| | | 9.85 (s, 1H), 8.38 (s, 1H), 8.02-7.95 | |
| | | (m, 1H), 7.73-7.71 (m, 1H), 7.52- | |
| | | 7.49 (m, 2H), 7.40-7.34 (m, 2H), | |
| | | 7.16-7.10 (m, 2H), 6.56-6.53 (m, | |
| | | 1H), 6.12 (d, J = 7.2 Hz, 1H), 5.21 (s, | |
| | | 2H), 4.41-4.35 (m, 2H), 3.90-3.81 | |
| | | (m, 3H), 3.67-3.60 (m, 4H), 3.25- | |
| | | 3.21 (m, 1H), 2.30-2.11 (m, 3H), | |
| | | 2.01-1.96 (m, 5H), 1.99-1.93 (m, | |
| | | 1H), 1.75-1.69 (m, 3H). | |
| 255 | 746.2 | ¹H NMR (500 MHz, Chloroform-d) δ | |
| | | 8.23 (d, J = 2.2 Hz, 1H), 8.12 (d, J = | |
| | | 1.9 Hz, 1H), 7.68-7.59 (m, 2H), 7.51- | |
| | | 7.49 (m, 1H), 7.43-7.36 (m, 3H), | |
| | | 7.19-7.12 (m, 2H), 6.84-6.82 (m, | |
| | | 1H), 6.66-6.63 (m, 1H), 5.39-5.34 | |
| | | (m, 2H), 4.47-4.41 (m, 3H), 3.80- | |
| | | 3.74 (m, 2H), 3.60-3.51 (m, 2H), | |
| | | 3.11-3.07 (m, 4H), 2.90 (s, 3H), 2.22- | |
| | | 2.15 (m, 2H). | |
| 256 | 741.2 | ¹H NMR (500 MHz, DMSO-d₆) δ | 4 |
| | | 12.69 (s, 1H), 10.89 (s, 1H), 8.26 (d, | |
| | | J = 2.2 Hz, 1H), 8.05 (d, J = 2.1 Hz, | |
| | | 1H), 7.80 (d, J = 2.2 Hz, 1H), 7.55- | |
| | | 7.53 (m, 1H), 7.53-7.46 (m, 2H), | |
| | | 7.49-7.45 (m, 1H), 7.46-7.40 (m, | |
| | | 1H), 7.35-7.26 (m, 2H), 6.89-6.83 | |
| | | (m, 1H), 6.58-6.52 (m, 1H), 5.34- | |
| | | 5.31 (m, 2H), 4.44-4.36 (m, 2H), | |
| | | 4.36-4.30 (m, 1H), 3.90-3.82 (m, | |
| | | 1H), 3.75-3.69 (m, 1H), 3.61-3.56 | |
| | | (m, 2H), 3.36-3.26 (m, 2H), 3.12- | |
| | | 3.02 (m, 2H), 2.72 (s, 3H), 2.45 (s, | |
| | | 3H), 2.38-2.33 (m, 1H), 2.09- | |
| | | 2.02 (m, 1H). | |
| 257 | 727.2 | ¹H NMR (500 MHz, DMSO-d₆) δ | 4 |
| | | 8.42 (d, J = 1.8 Hz, 1H), 8.05 (d, J = | |
| | | 2.1 Hz, 1H), 7.91-7.90 (m, 1H), | |
| | | 7.70 (d, J = 7.9 Hz, 1H), 7.55-7.53 | |
| | | (m, 1H), 7.53-7.46 (m, 2H), 7.49- | |
| | | 7.45 (m, 1H), 7.46-7.40 (m, 1H), | |
| | | 7.35-7.26 (m, 2H), 6.89-6.83 (m, | |
| | | 1H), 6.58-6.52 (m, 1H), 5.34-5.31 | |
| | | (m, 2H), 4.44-4.36 (m, 2H), 4.36- | |
| | | 4.30 (m, 1H), 3.90-3.82 (m, 1H), | |
| | | 3.75-3.69 (m, 1H), 3.61-3.56 (m, | |
| | | 2H), 3.36-3.26 (m, 2H), 3.13-3.02 | |
| | | (m, 2H), 2.43 (s, 3H), 2.36-2.28 (m, | |
| | | 1H), 2.09-2.02 (m, 1H). | |
| 258 | 753.2 | ¹H NMR (500 MHz, DMSO-d₆) δ | 4 |
| | | 8.49 (d, J = 1.4 Hz, 1H), 8.05 (d, J = | |
| | | 2.1 Hz, 1H), 7.92-7.91 (m, 1H), | |
| | | 7.77 (d, J = 7.9 Hz, 1H), 7.55-7.53 | |
| | | (m, 1H), 7.53-7.46 (m, 2H), 7.49- | |
| | | 7.45 (m, 1H), 7.46-7.40 (m, 1H), | |
| | | 7.35-7.26 (m, 2H), 6.89-6.83 (m, | |
| | | 1H), 6.58-6.52 (m, 1H), 5.34-5.31 | |
| | | (m, 2H), 4.44-4.36 (m, 2H), 4.35 (s, | |
| | | 1H), 4.38-4.30 (m, 1H), 3.90-3.82 | |
| | | (m, 1H), 3.75-3.69 (m, 1H), 3.61- | |
| | | 3.56 (m, 2H), 3.36-3.26 (m, 2H), | |
| | | 3.13-3.02 (m, 2H), 2.38-2.33 (m, | |
| | | 2H), 2.09-2.02 (m, 1H), 1.03-0.89 | |
| | | (m, 4H). | |
| 259 | 749.3 | ¹H NMR (500 MHz, DMSO-d₆) δ | 4 |
| | | 12.40 (s, 1H), 10.64 (s, 1H), 8.24 (d, | |
| | | J = 2.2 Hz, 1H), 7.98 (d, J = 2.1 Hz, | |
| | | 1H), 7.80-7.77 (m, 1H), 7.59-7.57 | |
| | | (m, 1H), 7.50 (t, J = 7.9 Hz, 1H), | |
| | | 7.49-7.45 (m, 3H), 7.35-7.26 (m, | |
| | | 2H), 7.19-7.18 (m, 1H), 7.40 (d, J = | |
| | | 1.7 Hz, 1H), 6.80-6.77 (m, 1H), | |
| | | 5.35-5.34 (m, 2H), 5.22 (s, 2H), 3.90- | |
| | | 3.83 (m, 2H), 3.71 (s, 2H), 3.11- | |
| | | 2.99 (m, 3H), 2.87-2.78 (m, 2H), | |
| | | 2.42 (s, 3H), 2.08-1.90 (m, 4H), | |
| | | 1.28 (t, J = 6.3 Hz, 3H). | |
| 260 | 695.3 | ¹H NMR (500 MHz, DMSO-d₆) δ | 4 |
| | | 12.83 (s, 1H), 10.46 (s, 1H), 8.32 (d, | |
| | | J = 2.2 Hz, 1H), 7.97 (d, J = 2.1 Hz, | |
| | | 1H), 7.80 (d, J = 2.0 Hz, 1H), 7.55- | |
| | | 7.53 (m, 1H), 7.50 (t, J = 7.9 Hz, | |
| | | 1H), 7.47 (s, 1H), 7.45 (d, J = 5.0 Hz, | |
| | | 1H), 7.35-7.26 (m, 2H), 7.19-7.18 | |
| | | (m, 1H), 6.80-6.77 (m, 1H), 5.35- | |
| | | 5.34 (m, 2H), 4.33 (t, J = 4.4 Hz, | |
| | | 2H), 3.73 (t, J = 4.4 Hz, 2H), 3.69 (s, | |
| | | 2H), 3.38 (s, 3H), 3.11-2.99 (m, | |
| | | 3H), 2.87-2.78 (m, 2H), 2.72 (s, | |
| | | 3H), 2.42 (s, 3H), 2.03-1.86 (m, | |
| | | 4H). | |
| 261 | 735.2 | ¹H NMR (500 MHz, DMSO-d₆) δ | 4 |
| | | 12.60 (s, 1H), 10.50 (s, 1H), 8.42 (d, | |
| | | J = 1.8 Hz, 1H), 7.97 (d, J = 2.1 Hz, | |
| | | 1H), 7.91-7.90 (m, 1H), 7.70 (d, J = | |
| | | 7.9 Hz, 1H), 7.55-7.53 (m, 1H), | |
| | | 7.50 (t, J = 7.9 Hz, 1H), 7.47 (s, 1H), | |
| | | 7.45 (s, 1H), 7.35-7.26 (m, 2H), | |
| | | 7.19-7.18 (m, 1H), 6.80-6.77 (m, | |
| | | 1H), 5.35-5.34 (m, 2H), 4.40 (t, J = | |
| | | 7.3 Hz, 2H), 3.92 (t, J = 7.3 Hz, 2H), | |
| | | 3.69 (s, 2H), 3.11-2.99 (m, 3H), | |
| | | 2.87-2.78 (m, 2H), 2.43 (s, 3H), | |
| | | 2.03-1.86 (m, 4H). | |
| 262 | 709.2 | ¹H NMR (500 MHz, DMSO-d₆) δ | 4 |
| | | 7.99-7.92 (m, 2H), 7.82 (d, J = 2.2 | |
| | | Hz, 1H), 7.75-7.72 (m, 1H), 7.57- | |
| | | 7.55 (s, 1H), 7.53-7.52 (m, 1H), 7.49- | |
| | | 7.40 (m, 2H), 7.35-7.26 (m, 2H), | |
| | | 6.86-6.69 (m, 2H), 5.34-5.31 (m, | |
| | | 2H), 4.38-4.24 (m, 2H), 4.10-3.88 | |
| | | (m, 4H), 3.77-3.70 (m, 1H), 3.35- | |
| | | 2.94 (m, 4H), 2.91-2.82 (m, 2H), | |
| | | 2.43 (s, 3H), 2.32-2.24 (m, 1H), | |
| | | 2.20-2.14 (m, 1H). | |
| 263 | 691.3 | ¹H NMR (500 MHz, DMSO-d₆) δ | 3 |
| | | 12.67 (s, 1H), 10.69 (s, 1H), 7.99- | |
| | | 7.92 (m, 2H), 7.88 (d, J = 2.2 Hz, | |
| | | 1H), 7.75-7.72 (m, 1H), 7.68- | |
| | | 7.66 (m, 1H), 7.53-7.49 (m, 3H), | |
| | | 7.43-7.32 (m, 1H), 7.35-7.26 (m, | |
| | | 2H), 6.86-6.69 (m, 2H), 5.34-5.31 | |
| | | (m, 2H), 4.38-4.24 (m, 2H), 4.10- | |
| | | 3.88 (m, 4H), 3.77-3.70 (m, 1H), | |
| | | 3.35-2.94 (m, 4H), 2.91-2.85 (m, | |
| | | 2H), 2.80 (s, 3H), 2.32-2.24 (m, | |
| | | 1H), 2.22-2.17 (m, 1H). | |
| 264 | 705.3 | ¹H NMR (500 MHz, DMSO-d₆) δ | 33 |
| | | 12.44 (s, 1H), 10.98 (s, 1H), 8.04- | |
| | | 8.02 (m, 1H), 7.89-7.84 (m, 2H), | |
| | | 7.70 (d, J = 2.2 Hz, 1H), 7.53-7.49 | |
| | | (m, 3H), 7.43-7.32 (m, 1H), 7.35- | |
| | | 7.26 (m, 2H), 6.86-6.69 (m, 2H), | |
| | | 5.34-5.31 (m, 2H), 4.38-4.24 (m, | |
| | | 2H), 4.10-3.88 (m, 4H), 3.77-3.70 | |
| | | (m, 1H), 3.35-2.94 (m, 4H), 2.91- | |
| | | 2.82 (m, 2H), 2.73 (s, 3H), 2.46 (s, | |
| | | 3H), 2.32-2.24 (m, 1H), 2.20-2.14 | |
| | | (m, 1H). | |
| 265 | 695.2 | ¹H NMR (500 MHz, DMSO-d₆) δ | |
| | | 8.18-8.16 (m, 1H), 8.04-8.02 (m, | |
| | | 1H), 7.97-7.94 (m, 1H), 7.75- | |
| | | 7.72 (m, 1H), 7.70-7.62 (m, 2H), | |
| | | 7.53-7.52 (m, 1H), 7.49-7.40 (m, | |
| | | 2H), 7.35-7.26 (m, 2H), 6.86-6.69 | |
| | | (m, 2H), 5.34-5.31 (m, 2H), 4.38- | |
| | | 4.24 (m, 2H), 4.10-3.88 (m, 4H), | |
| | | 3.77-3.70 (m, 1H), 3.35-2.94 (m, | |
| | | 4H), 2.91-2.82 (m, 2H), 2.32-2.24 | |
| | | (m, 1H), 2.20-2.14 (m, 1H). | |
| 266 | 718.3 | ¹H NMR (500 MHz, DMSO-d₆) δ | 3 |
| | | 10.34 (s, 1H), 10.20 (s, 1H), 7.99 (d, | |
| | | J = 1.7 Hz, 1H), 7.95-7.70 (m, 3H), | |
| | | 7.62 (m, 1H), 7.53-7.52 (m, 1H), 7.49- | |
| | | 7.40 (m, 2H), 7.35-7.19 (m, 3H), | |
| | | 6.86-6.69 (m, 2H), 5.34-5.31 (m, | |
| | | 2H), 4.37-4.26 (m, 2H), 4.06-3.99 | |
| | | (m, 1H), 3.77-3.68 (m, 4H), 3.35- | |
| | | 2.94 (m, 4H), 2.91-2.82 (m, 2H), | |
| | | 2.30-2.18 (m, 2H), 2.15-1.91 (m, | |
| | | 4H). | |
| 267 | 732.3 | ¹H NMR (500 MHz, DMSO-d₆) δ | 3 |
| | | 10.19 (s, 1H), 9.93 (s, 1H), 7.99 (d, J = | |
| | | 1.7 Hz, 1H), 7.97-7.95 (m, 1H), | |
| | | 7.75-7.72 (m, 1H), 7.65 (d, J = 2.2 | |
| | | Hz, 1H), 7.57-7.50 (m, 2H), 7.49- | |
| | | 7.19 (m, 4H), 6.86-6.69 (m, 2H), | |
| | | 5.34-5.31 (m, 2H), 4.37-4.26 (m, | |
| | | 2H), 4.06-3.99 (m, 1H), 3.77-3.68 | |
| | | (m, 4H), 3.35-2.94 (m, 4H), 2.91- | |
| | | 2.82 (m, 2H), 2.30-2.20 (m, 2H), | |
| | | 2.22 (s, 3H), 2.18-2.08 (m, 2H), 1.72- | |
| | | 1.61 (m, 2H). | |
| 268 | 681.2 | ¹H NMR (500 MHz, DMSO-d₆) δ | 3 |
| | | 12.79 (s, 1H), 8.23 (d, J = 1.8 Hz, | |
| | | 1H), 7.53-7.52 (m, 1H), 7.49-7.40 | |
| | | (m, 2H), 7.42-7.36 (m, 2H), 7.34- | |
| | | 7.28 (m, 2H), 7.25-7.19 (m, 1H), | |
| | | 6.91-6.83 (m, 2H), 6.47-6.45 (m, | |
| | | 1H), 6.28-6.23 (m, 1H), 6.16 (t, J = | |
| | | 5.3 Hz, 1H), 5.34-5.31 (m, 2H), 4.51- | |
| | | 4.40 (m, 2H), 4.37-4.26 (m, 2H), | |
| | | 4.06-3.99 (m, 1H), 3.77-3.68 (m, | |
| | | 4H), 3.35-2.94 (m, 4H), 2.91-2.82 | |
| | | (m, 2H), 2.19-2.10 (m, 2H). | |
| 269 | 711.2 | ¹H NMR (500 MHz, DMSO-d₆) δ | 3 |
| | | 8.09 (d, J = 2.4 Hz, 1H), 8.04-8.02 | |
| | | (m, 1H), 7.97-7.95 (m, 1H), 7.75- | |
| | | 7.72 (m, 1H), 7.66-7.58 (m, 2H), | |
| | | 7.53-7.52 (m, 1H), 7.49-7.40 (m, | |
| | | 2H), 7.35-7.26 (m, 2H), 6.86-6.69 | |
| | | (m, 2H), 5.34-5.31 (m, 2H), 4.38- | |
| | | 4.24 (m, 2H), 4.10-3.88 (m, 4H), | |
| | | 3.77-3.70 (m, 1H), 3.35-2.94 (m, | |
| | | 4H), 2.91-2.82 (m, 2H), 2.32-2.24 | |
| | | (m, 1H), 2.20-2.14 (m, 1H). | |
| 270 | 693.2 | ¹H NMR (500 MHz, DMSO-d₆) δ | 3 |
| | | 10.54 (s, 2H), 10.16 (s, 1H), 8.31 (d, | |
| | | J = 2.1 Hz, 1H), 8.04-8.02 (m, 1H), | |
| | | 7.97-7.95 (m, 1H), 7.86 (d, J = 8.0 | |
| | | Hz, 1H), 7.75-7.72 (m, 1H), 7.53- | |
| | | 7.45 (m, 2H), 7.49-7.40 (m, 2H), | |
| | | 7.35-7.26 (m, 2H), 6.86-6.69 (m, | |
| | | 2H), 5.34-5.31 (m, 2H), 4.37-4.26 | |
| | | (m, 2H), 4.06-3.95 (m, 2H), 3.95- | |
| | | 3.82 (m, 2H), 3.78-3.69 (m, 1H), | |
| | | 3.35-2.94 (m, 4H), 2.91-2.82 (m, | |
| | | 2H), 2.38-2.33 (m, 1H), 2.09- | |
| | | 2.02 (m, 1H). | |
| 271 | 736.2 | ¹H NMR (500 MHz, DMSO-d₆) δ | 3 |
| | | 10.35 (s, 1H), 9.97 (s, 1H), 7.99 (d, J = | |
| | | 1.7 Hz, 1H), 7.97-7.95 (m, 1H), | |
| | | 7.75-7.72 (m, 1H), 7.63-7.56 (m, | |
| | | 2H), 7.53-7.52 (m, 1H), 7.49-7.40 | |
| | | (m, 2H), 7.35-7.26 (m, 2H), 6.86- | |
| | | 6.69 (m, 2H), 5.34-5.31 (m, 2H), 4.37- | |
| | | 4.26 (m, 2H), 4.06-3.99 (m, 1H), | |
| | | 3.77-3.68 (m, 4H), 3.35-2.94 (m, | |
| | | 4H), 2.91-2.82 (m, 2H), 2.30-2.18 | |
| | | (m, 2H), 2.21-2.13 (m, 1H), 2.15- | |
| | | 2.08 (m, 1H), 1.73-1.62 (m, 2H). | |
| 272 | 759.2 | ¹H NMR (500 MHz, DMSO-d₆) δ | 3 |
| | | 12.65 (s, 1H), 10.77 (s, 1H), 8.08 (d, | |
| | | J = 2.2 Hz, 1H), 8.04-8.02 (m, 1H), | |
| | | 7.95-7.86 (m, 2H), 7.75-7.72 (m, | |
| | | 1H), 7.53-7.52 (m, 1H), 7.49-7.40 | |
| | | (m, 2H), 7.35-7.26 (m, 2H), 6.86- | |
| | | 6.69 (m, 2H), 5.34-5.31 (m, 2H), 4.38- | |
| | | 4.24 (m, 2H), 4.10-3.88 (m, 4H), | |
| | | 3.77-3.70 (m, 1H), 3.35-2.94 (m, | |
| | | 4H), 2.91-2.82 (m, 2H), 2.46 (s, | |
| | | 3H), 2.32-2.24 (m, 1H), 2.20-2.14 | |
| | | (m, 1H). | |
| 273 | 786.2 | ¹H NMR (500 MHz, DMSO-d₆) δ | 3 |
| | | 10.23 (s, 1H), 9.94 (s, 1H), 8.11 (d, J = | |
| | | 2.2 Hz, 1H), 7.99 (d, J = 1.7 Hz, | |
| | | 1H), 7.97-7.95 (m, 1H), 7.75- | |
| | | 7.72 (m, 1H), 7.62 (d, J = 2.2 Hz, | |
| | | 1H), 7.53-7.52 (m, 1H), 7.49-7.40 | |
| | | (m, 2H), 7.35-7.26 (m, 2H), 6.86- | |
| | | 6.69 (m, 2H), 5.34-5.31 (m, 2H), 4.37- | |
| | | 4.26 (m, 2H), 4.06-3.99 (m, 1H), | |
| | | 3.77-3.68 (m, 4H), 3.35-2.94 (m, | |
| | | 4H), 2.91-2.82 (m, 2H), 2.19-2.10 | |
| | | (m, 2H), 2.15-2.04 (m, 4H). | |
| 274 | 700.3 | ¹H NMR (500 MHz, DMSO-d₆) δ | 3 |
| | | 12.44 (s, 1H), 11.01 (s, 1H), 7.99- | |
| | | 7.92 (m, 2H), 7.82 (d, J = 2.2 Hz, | |
| | | 1H), 7.75-7.72 (m, 1H), 7.63- | |
| | | 7.59 (m, 1H), 7.57-7.45 (m, 4H), | |
| | | 7.45-7.40 (m, 1H), 6.86-6.69 (m, | |
| | | 2H), 5.34-5.31 (m, 2H), 4.38-4.24 | |
| | | (m, 2H), 4.10-3.88 (m, 4H), 3.77- | |
| | | 3.70 (m, 1H), 3.35-2.94 (m, 4H), | |
| | | 2.91-2.82 (m, 2H), 2.43 (s, 3H), | |
| | | 2.32-2.24 (m, 1H), 2.20-2.14 (m, | |
| | | 1H). | |
| 275 | 682.3 | ¹H NMR (500 MHz, DMSO-d₆) δ | 3 |
| | | 12.67 (s, 1H), 10.69 (s, 1H), 7.99- | |
| | | 7.92 (m, 2H), 7.88 (d, J = 2.2 Hz, | |
| | | 1H), 7.75-7.72 (m, 1H), 7.68- | |
| | | 7.66 (m, 1H), 7.62-7.57 (m, 1H), | |
| | | 7.56-7.47 (m, 2H), 7.51-7.40 (m, | |
| | | 3H), 6.86-6.69 (m, 2H), 5.34-5.31 | |
| | | (m, 2H), 4.38-4.24 (m, 2H), 4.10- | |
| | | 3.88 (m, 4H), 3.77-3.70 (m, 1H), | |
| | | 3.35-2.94 (m, 4H), 2.91-2.82 (m, | |
| | | 2H), 2.84 (s, 3H), 2.32-2.24 (m, | |
| | | 1H), 2.20-2.14 (m, 1H). | |
| 276 | 696.3 | ¹H NMR (500 MHz, DMSO-d₆) δ | 3 |
| | | 12.00 (s, 1H), 10.88 (s, 1H), 8.04- | |
| | | 8.02 (m, 1H), 7.89-7.84 (m, 2H), | |
| | | 7.70 (d, J = 2.2 Hz, 1H), 7.63-7.59 | |
| | | (m, 1H), 7.56-7.52 (m, 1H), 7.52- | |
| | | 7.47 (m, 2H), 7.50-7.45 (m, 1H), | |
| | | 7.45-7.40 (m, 1H), 6.86-6.69 (m, | |
| | | 2H), 5.34-5.31 (m, 2H), 4.38-4.24 | |
| | | (m, 2H), 4.10-3.88 (m, 4H), 3.77- | |
| | | 3.70 (m, 1H), 3.35-2.94 (m, 4H), | |
| | | 2.91-2.82 (m, 2H), 2.73 (s, 3H), | |
| | | 2.46 (s, 3H), 2.32-2.24 (m, 1H), | |
| | | 2.20-2.14 (m, 1H). | |
| 277 | 709.2 | ¹H NMR (500 MHz, DMSO-d₆) δ | 3 |
| | | 12.40 (s, 1H), 10.28 (s, 1H), 8.24 (d, | |
| | | J = 2.2 Hz, 1H), 7.77-7.75 (m, 2H), | |
| | | 7.69-7.65 (m, 1H), 7.53-7.49 (m, | |
| | | 3H), 7.43-7.32 (m, 1H), 7.35- | |
| | | 7.26 (m, 2H), 6.86-6.69 (m, 2H), | |
| | | 5.34-5.31 (m, 2H), 4.38-4.24 (m, | |
| | | 2H), 4.10-3.88 (m, 4H), 3.77-3.70 | |
| | | (m, 1H), 3.35-2.94 (m, 4H), 2.91- | |
| | | 2.82 (m, 2H), 2.42 (s, 3H), 2.32- | |
| | | 2.24 (m, 1H), 2.20-2.14 (m, 1H). | |
| 278 | 691.3 | ¹H NMR (500 MHz, DMSO-d₆) δ | 3 |
| | | 12.60 (s, 1H), 10.27 (s, 1H), 8.42 (d, | |
| | | J = 1.8 Hz, 1H), 7.91-7.90 (m, 1H), | |
| | | 7.75 (d, J = 2.2 Hz, 1H), 7.73-7.64 | |
| | | (m, 2H), 7.53-7.49 (m, 3H), | |
| | | 7.43-7.32 (m, 1H), 7.35-7.26 (m, | |
| | | 2H), 6.86-6.69 (m, 2H), 5.34-5.31 | |
| | | (m, 2H), 4.38-4.24 (m, 2H), 4.10- | |
| | | 3.88 (m, 4H), 3.77-3.70 (m, 1H), | |
| | | 3.35-2.94 (m, 4H), 2.91-2.82 (m, | |
| | | 2H), 2.43 (s, 3H), 2.32-2.24 (m, | |
| | | 1H), 2.20-2.14 (m, 1H). | |
| 279 | 705.3 | ¹H NMR (500 MHz, DMSO-d₆) δ | 3 |
| | | 12.85 (s, 1H), 10.29 (s, 1H), 8.32 (d, | |
| | | J = 2.2 Hz, 1H), 7.80 (d, J = 2.2 Hz, | |
| | | 1H), 7.75 (d, J = 2.2 Hz, 1H), 7.70- | |
| | | 7.66 (m, 1H), 7.53-7.49 (m, 3H), | |
| | | 7.43-7.32 (m, 1H), 7.35-7.26 (m, | |
| | | 2H), 6.86-6.69 (m, 2H), 5.34-5.31 | |
| | | (m, 2H), 4.38-4.24 (m, 2H), 4.10- | |
| | | 3.88 (m, 4H), 3.77-3.70 (m, 1H), | |
| | | 3.35-2.94 (m, 4H), 2.91-2.82 (m, | |
| | | 2H), 2.72 (s, 3H), 2.45 (s, 3H), 2.32- | |
| | | 2.24 (m, 1H), 2.20-2.14 (m, 1H). | |
| 280 | 695.2 | ¹H NMR (500 MHz, DMSO-d₆) δ | 3 |
| | | 12.97 (s, 1H), 10.36 (s, 1H), 8.63- | |
| | | 8.58 (m, 1H), 7.97-7.95 (m, 1H), | |
| | | 7.78-7.73 (m, 2H), 7.70-7.66 (m, | |
| | | 1H), 7.53-7.49 (m, 3H), 7.43- | |
| | | 7.32 (m, 1H), 7.35-7.26 (m, 2H), | |
| | | 6.86-6.69 (m, 2H), 5.34-5.31 (m, | |
| | | 2H), 4.38-4.24 (m, 2H), 4.10-3.88 | |
| | | (m, 4H), 3.77-3.70 (m, 1H), 3.35- | |
| | | 2.94 (m, 4H), 2.91-2.82 (m, 2H), | |
| | | 2.32-2.24 (m, 1H), 2.20-2.14 (m, | |
| | | 1H). | |
| 281 | 718.3 | ¹H NMR (500 MHz, DMSO-d₆) δ | 3 |
| | | 10.13 (s, 1H), 9.90 (s, 1H), 8.09 (d, J = | |
| | | 1.7 Hz, 1H), 7.92-7.91 (m, 1H), | |
| | | 7.75 (d, J = 2.1 Hz, 1H), 7.70-7.66 | |
| | | (m, 1H), 7.53-7.43 (m, 3H), 7.46- | |
| | | 7.40 (m, 1H) 7.33-7.28 (m, 1H), | |
| | | 7.25-7.16 (m, 2H), 6.86-6.69 (m, | |
| | | 2H), 5.34-5.31 (m, 2H), 4.37-4.26 | |
| | | (m, 2H), 4.06-3.99 (m, 1H), 3.77- | |
| | | 3.68 (m, 4H), 3.35-2.94 (m, 4H), | |
| | | 2.91-2.82 (m, 2H), 2.30-2.18 (m, | |
| | | 2H), 2.21-2.12 (m, 1H), 2.11-1.97 | |
| | | (m, 1H), 1.72-1.61 (m, 2H). | |
| 282 | 732.3 | ¹H NMR (500 MHz, DMSO-d₆) δ | 3 |
| | | 10.07 (s, 1H), 9.93 (s, 1H), 7.95 (d, J = | |
| | | 2.2 Hz, 1H), 7.88 (d, J = 2.1 Hz, | |
| | | 1H), 7.75 (d, J = 2.1 Hz, 1H), 7.70- | |
| | | 7.66 (m, 1H), 7.53-7.49 (m, 3H), | |
| | | 7.43-7.32 (m, 1H), 7.35-7.26 (m, | |
| | | 2H), 6.86-6.69 (m, 2H), 5.34-5.31 | |
| | | (m, 2H), 4.37-4.26 (m, 2H), 4.06- | |
| | | 3.99 (m, 1H), 3.77-3.68 (m, 4H), | |
| | | 3.35-2.94 (m, 4H), 2.91-2.82 (m, | |
| | | 2H), 2.30-2.18 (m, 2H), 2.21 (s, | |
| | | 3H), 2.18-1.72 (m, 4H). | |
| 283 | 695.2 | ¹H NMR (500 MHz, DMSO-d₆) δ | 3 |
| | | 8.50 (t, J = 2.0 Hz, 1H), 8.35 (d, J = | |
| | | 1.8 Hz, 1H), 7.83-7.80 (m, 1H), | |
| | | 7.75 (d, J = 2.1 Hz, 1H), 7.69-7.65 | |
| | | (m, 1H), 7.53-7.49 (m, 3H), | |
| | | 7.43-7.32 (m, 1H), 7.35-7.26 (m, | |
| | | 2H), 6.86-6.69 (m, 2H), 5.34-5.31 | |
| | | (m, 2H), 4.37-4.26 (m, 2H), 4.06- | |
| | | 3.99 (m, 1H), 3.77-3.68 (m, 4H), | |
| | | 3.35-2.94 (m, 4H), 2.91-2.82 (m, | |
| | | 2H), 2.19-2.10 (m, 2H). | |
| 284 | 711.2 | ¹H NMR (500 MHz, DMSO-d₆) δ | 3 |
| | | 12.87 (s, 1H), 10.21 (s, 1H), 8.49 (d, | |
| | | J = 1.4 Hz, 1H), 7.93-7.91 (m, 1H), | |
| | | 7.77-7.73 (m, 2H), 7.70-7.66 (m, | |
| | | 1H), 7.53-7.49 (m, 3H), 7.43- | |
| | | 7.32 (m, 1H), 7.35-7.26 (m, 2H), | |
| | | 6.86-6.69 (m, 2H), 5.34-5.31 (m, | |
| | | 2H), 4.38-4.24 (m, 2H), 4.10-3.88 | |
| | | (m, 4H), 3.77-3.70 (m, 1H), 3.35- | |
| | | 2.94 (m, 4H), 2.91-2.82 (m, 2H), | |
| | | 2.32-2.24 (m, 1H), 2.20-2.14 (m, | |
| | | 1H). | |
| 285 | 693.2 | ¹H NMR (500 MHz, DMSO-d₆) δ | 3 |
| | | 8.57 (d, J = 2.1 Hz, 1H), 7.82-7.80 | |
| | | (m, 1H), 7.75 (d, J = 2.2 Hz, 1H), | |
| | | 7.70-7.66 (m, 1H), 7.53-7.40 (m, | |
| | | 5H), 7.35-7.26 (m, 2H), 6.86-6.69 | |
| | | (m, 2H), 5.34-5.31 (m, 2H), 4.37- | |
| | | 4.26 (m, 2H), 4.06-3.82 (m, 4H), | |
| | | 3.75-3.69 (m, 1H), 3.35-2.94 (m, | |
| | | 4H), 2.91-2.82 (m, 2H), 2.38-2.33 | |
| | | (m, 1H), 2.09-2.02 (m, 1H). | |
| 286 | 736.2 | ¹H NMR (500 MHz, DMSO-d₆) δ | 3 |
| | | 10.17 (s, 1H), 10.03 (s, 1H), 7.90 (d, | |
| | | J = 2.2 Hz, 1H), 7.84-7.81 (m, 1H), | |
| | | 7.75 (d, J = 2.1 Hz, 1H), 7.69-7.65 | |
| | | (m, 1H), 7.53-7.42 (m, 3H), 7.46- | |
| | | 7.40 (m, 1H), 7.35-7.26 (m, 2H), | |
| | | 6.86-6.69 (m, 2H), 5.34-5.31 (m, | |
| | | 2H), 4.37-4.26 (m, 2H), 4.06-3.99 | |
| | | (m, 1H), 3.77-3.68 (m, 4H), 3.35- | |
| | | 2.94 (m, 4H), 2.91-2.82 (m, 2H), | |
| | | 2.30-2.18 (m, 2H), 2.21-1.69 (m, | |
| | | 4H). | |
| 287 | 759.2 | ¹H NMR (500 MHz, DMSO-d₆) δ | 3 |
| | | 8.38-8.37 (m, 1H), 8.27 (d, J = 2.0 | |
| | | Hz, 1H), 7.75 (d, J = 2.2 Hz, 1H), | |
| | | 7.70-7.66 (m, 1H), 7.53-7.49 (m, | |
| | | 3H), 7.43-7.32 (m, 1H), 7.35- | |
| | | 7.26 (m, 2H), 6.86-6.69 (m, 2H), | |
| | | 5.34-5.31 (m, 2H), 4.38-4.24 (m, | |
| | | 2H), 4.10-3.88 (m, 4H), 3.77-3.70 | |
| | | (m, 1H), 3.35-2.94 (m, 4H), 2.91- | |
| | | 2.82 (m, 2H), 2.45 (s, 3H), 2.32- | |
| | | 2.24 (m, 1H), 2.20-2.14 (m, 1H). | |
| 288 | 786.2 | ¹H NMR (500 MHz, DMSO-d₆) δ | 3 |
| | | 10.36 (s, 1H), 9.85 (s, 1H), 8.25 (d, J = | |
| | | 2.2 Hz, 1H), 8.08 (d, J = 2.2 Hz, | |
| | | 1H), 7.75 (d, J = 2.1 Hz, 1H), 7.70- | |
| | | 7.66 (m, 1H), 7.53-7.49 (m, 3H), | |
| | | 7.43-7.32 (m, 1H), 7.35-7.26 (m, | |
| | | 2H), 6.86-6.69 (m, 2H), 5.34-5.31 | |
| | | (m, 2H), 4.37-4.26 (m, 2H), 4.06- | |
| | | 3.99 (m, 1H), 3.77-3.68 (m, 4H), | |
| | | 3.35-2.94 (m, 4H), 2.91-2.82 (m, | |
| | | 2H), 2.30-2.18 (m, 2H), 2.22-1.72 | |
| | | (m, 4H). | |
| 289 | 700.3 | ¹H NMR (500 MHz, DMSO-d₆) δ | 3 |
| | | 12.43 (s, 1H), 10.28 (s, 1H), 8.24 (d, | |
| | | J = 2.2 Hz, 1H), 7.80-7.77 (m, 1H), | |
| | | 7.75 (d, J = 2.2 Hz, 1H), 7.69-7.65 | |
| | | (m, 1H), 7.63-7.59 (m, 1H), 7.56- | |
| | | 7.52 (m, 1H), 7.52-7.44 (m, 3H), | |
| | | 7.45-7.43 (m, 1H), 6.86-6.69 (m, | |
| | | 2H), 5.34-5.31 (m, 2H), 4.38-4.24 | |
| | | (m, 2H), 4.10-3.88 (m, 4H), 3.77- | |
| | | 3.70 (m, 1H), 3.35-2.94 (m, 4H), | |
| | | 2.91-2.82 (m, 2H), 2.42 (s, 3H), | |
| | | 2.32-2.24 (m, 1H), 2.20-2.14 (m, | |
| | | 1H). | |
| 290 | 682.3 | ¹H NMR (500 MHz, DMSO-d₆) δ | 3 |
| | | 8.42 (d, J = 1.8 Hz, 1H), 7.91-7.90 | |
| | | (m, 1H), 7.77 (d, J = 2.2 Hz, 1H), | |
| | | 7.70-7.64 (m, 2H), 7.61-7.58 (m, | |
| | | 1H), 7.56-7.52 (m, 1H), 7.52-7.44 | |
| | | (m, 3H), 7.45-7.43 (m, 1H), 6.86- | |
| | | 6.69 (m, 2H), 5.34-5.31 (m, 2H), 4.38- | |
| | | 4.24 (m, 2H), 4.10-3.88 (m, 4H), | |
| | | 3.77-3.70 (m, 1H), 3.35-2.94 (m, | |
| | | 4H), 2.91-2.82 (m, 2H), 2.43 (s, | |
| | | 3H), 2.32-2.24 (m, 1H), 2.20-2.14 | |
| | | (m, 1H). | |
| 291 | 696.3 | ¹H NMR (500 MHz, DMSO-d₆) δ | 3 |
| | | 12.45 (s, 1H), 10.27 (s, 1H), 8.32 (d, | |
| | | J = 2.2 Hz, 1H), 7.80 (d, J = 2.2 Hz, | |
| | | 1H), 7.75 (d, J = 2.2 Hz, 1H), 7.70- | |
| | | 7.66 (m, 1H), 7.63-7.59 (m, 1H), | |
| | | 7.56-7.52 (m, 1H), 7.52-7.47 (m, | |
| | | 2H), 7.50-7.44 (m, 1H), 7.47-7.40 | |
| | | (m, 1H), 6.86-6.69 (m, 2H), 5.34- | |
| | | 5.31 (m, 2H), 4.38-4.24 (m, 2H), | |
| | | 4.10-3.88 (m, 4H), 3.77-3.70 (m, | |
| | | 1H), 3.35-2.94 (m, 4H), 2.91-2.82 | |
| | | (m, 2H), 2.72 (s, 3H), 2.45 (s, 3H), | |
| | | 2.32-2.24 (m, 1H), 2.20-2.14 (m, | |
| | | 1H). | |
| 292 | 722.2 | ¹H NMR (500 MHz, DMSO-d₆) δ | 5 |
| | | 12.92 (s, 1H), 10.71 (s, 1H), 8.22- | |
| | | 8.14 (m, 2H), 7.92-7.89 (m, 1H), | |
| | | 7.69-7.62 (m, 2H), 7.62-7.59 (m, | |
| | | 1H), 7.47-7.40 (m, 2H), 7.25-6.98 | |
| | | (m, 4H), 6.11-6.08 (m, 1H), 4.37- | |
| | | 4.33 (m, 1H), 4.32-4.28 (m, 1H), | |
| | | 4.08-3.80 (m, 3H), 3.80-3.67 (m, | |
| | | 2H), 3.35-3.27 (m, 2H), 2.89-2.81 | |
| | | (m, 2H), 2.80-2.72 (m, 2H), 2.38- | |
| | | 2.33 (m, 1H), 2.09-2.02 (m, 1H), | |
| | | 1.99 (s, 3H). | |
| 293 | 752.2 | ¹H NMR (500 MHz, DMSO-d₆) δ | 5 |
| | | 12.40 (s, 1H), 10.75 (s, 1H), 8.23 (d, | |
| | | J = 2.1 Hz, 1H), 7.92-7.91 (m, 1H), | |
| | | 7.87-7.82 (m, 2H), 7.62-7.59 (m, | |
| | | 1H), 7.47-7.40 (m, 2H), 7.25-6.98 | |
| | | (m, 4H), 6.11-6.08 (m, 1H), 4.37- | |
| | | 4.33 (m, 1H), 4.32-4.28 (m, 1H), | |
| | | 4.08-3.80 (m, 3H), 3.80-3.67 (m, | |
| | | 2H), 3.35-3.27 (m, 2H), 2.89-2.81 | |
| | | (m, 2H), 2.80-2.72 (m, 2H), 2.44 (s, | |
| | | 3H), 2.38-2.33 (m, 1H), 2.09- | |
| | | 2.02 (m, 1H), 1.99 (s, 3H). | |
| 294 | 744.3 | ¹H NMR (500 MHz, DMSO-d₆) δ | 5 |
| | | 12.64 (s, 1H), 10.83 (s, 1H), 8.23 (d, | |
| | | J = 2.1 Hz, 1H), 8.15-8.11 (m, 1H), | |
| | | 7.92-7.89 (m, 1H), 7.70-7.66 (m, | |
| | | 1H), 7.62-7.55 (m, 2H), 7.47-7.40 | |
| | | (m, 2H), 7.25-6.98 (m, 4H), 6.11- | |
| | | 6.08 (m, 1H), 4.37-4.33 (m, 1H), | |
| | | 4.32-4.28 (m, 1H), 4.08-3.80 (m, | |
| | | 3H), 3.80-3.67 (m, 2H), 3.35-3.27 | |
| | | (m, 2H), 2.89-2.81 (m, 2H), 2.80- | |
| | | 2.72 (m, 2H), 2.38-2.33 (m, 2H), | |
| | | 2.09-1.98 (m, 4H), 0.96-0.66 (m, | |
| | | 4H). | |
| 295 | 786.2 | ¹H NMR (500 MHz, DMSO-d₆) δ | 5 |
| | | 12.71 (s, 1H), 10.84 (s, 1H), 8.23 (d, | |
| | | J = 2.1 Hz, 1H), 8.08 (d, J = 2.2 Hz, | |
| | | 1H), 7.92-7.91 (m, 1H), 7.86 (d, J = | |
| | | 2.2 Hz, 1H), 7.62-7.59 (m, 1H), | |
| | | 7.47-7.40 (m, 2H), 7.25-6.98 (m, | |
| | | 4H), 6.11-6.08 (m, 1H), 4.37-4.33 | |
| | | (m, 1H), 4.32-4.28 (m, 1H), 4.08- | |
| | | 3.80 (m, 3H), 3.80-3.67 (m, 2H), | |
| | | 3.35-3.27 (m, 2H), 2.89-2.81 (m, | |
| | | 2H), 2.80-2.72 (m, 2H), 2.46 (s, | |
| | | 3H), 2.38-2.33 (m, 1H), 2.09- | |
| | | 2.02 (m, 1H), 1.99 (s, 3H). | |
| 296 | 786.2 | ¹H NMR (500 MHz, DMSO-d₆) δ | 5 |
| | | 12.60 (s, 1H), 11.11 (s, 1H), 8.23 (d, | |
| | | J = 2.1 Hz, 1H), 7.92-7.89 (m, 2H), | |
| | | 7.62-7.59 (m, 1H), 7.49 (d, J = 2.2 | |
| | | Hz, 1H), 7.47-7.40 (m, 2H), 7.25- | |
| | | 6.98 (m, 4H), 6.11-6.08 (m, 1H), | |
| | | 4.37-4.33 (m, 1H), 4.32-4.28 (m, | |
| | | 1H), 4.08-3.80 (m, 3H), 3.80-3.67 | |
| | | (m, 2H), 3.35-3.27 (m, 2H), 2.89- | |
| | | 2.81 (m, 2H), 2.80-2.72 (m, 2H), | |
| | | 2.74 (s, 2H), 2.38-2.33 (m, 1H), | |
| | | 2.09-2.02 (m, 1H), 1.92 (s, 3H). | |
| 297 | 772.2 | ¹H NMR (500 MHz, DMSO-d₆) δ | 5 |
| | | 13.03 (s, 1H), 10.94 (s, 1H), 8.23 (d, | |
| | | J = 2.1 Hz, 1H), 8.12 (d, J = 2.2 Hz, | |
| | | 1H), 7.92-7.89 (m, 1H), 7.67-7.65 | |
| | | (m, 1H), 7.63-7.56 (m, 2H), 7.47- | |
| | | 7.40 (m, 2H), 7.25-6.98 (m, 4H), | |
| | | 6.11-6.08 (m, 1H), 4.37-4.33 (m, | |
| | | 1H), 4.32-4.28 (m, 1H), 4.08-3.80 | |
| | | (m, 3H), 3.80-3.67 (m, 2H), 3.35- | |
| | | 3.27 (m, 2H), 2.89-2.81 (m, 2H), | |
| | | 2.80-2.72 (m, 2H), 2.38-2.33 (m, | |
| | | 1H), 2.09-2.02 (m, 1H), 1.99 (s, | |
| | | 3H). | |
| 298 | 727.2 | ¹H NMR (500 MHz, DMSO-d₆) δ | 5 |
| | | 12.44 (s, 1H), 10.82 (s, 1H), 8.23 (d, | |
| | | J = 2.1 Hz, 1H), 7.92-7.89 (m, 1H), | |
| | | 7.82 (d, J = 2.2 Hz, 1H), 7.79-7.73 | |
| | | (m, 2H), 7.63-7.59 (m, 1H), 7.57- | |
| | | 7.52 (m, 1H), 7.42 (d, J = 8.8 Hz, | |
| | | 1H), 7.20-7.16 (m, 1H), 7.05-6.98 | |
| | | (m, 2H), 6.11-6.08 (m, 1H), 4.37- | |
| | | 4.33 (m, 1H), 4.32-4.28 (m, 1H), | |
| | | 4.08-3.80 (m, 3H), 3.80-3.67 (m, | |
| | | 2H), 3.35-3.27 (m, 2H), 2.89-2.81 | |
| | | (m, 2H), 2.80-2.72 (m, 2H), 2.43 (s, | |
| | | 3H), 2.38-2.33 (m, 1H), 2.09- | |
| | | 2.02 (m, 1H), 1.82 (s, 3H). | |
| 299 | 723.3 | ¹H NMR (500 MHz, DMSO-d₆) δ | 5 |
| | | 8.23 (d, J = 2.1 Hz, 1H), 7.92-7.89 | |
| | | (m, 1H), 7.78-7.73 (m, 1H), 7.70 (d, | |
| | | J = 2.2 Hz, 1H), 7.48-7.47 (m, 1H), | |
| | | 7.42 (d, J = 8.8 Hz, 1H), 7.20-7.16 | |
| | | (m, 1H), 7.05-6.98 (m, 3H), 6.98- | |
| | | 6.93 (m, 1H), 6.11-6.08 (m, 1H), | |
| | | 4.37-4.33 (m, 1H), 4.32-4.28 (m, | |
| | | 1H), 4.08-3.80 (m, 3H), 3.80-3.67 | |
| | | (m, 2H), 3.35-3.27 (m, 2H), 2.89- | |
| | | 2.81 (m, 2H), 2.80-2.74 (m, 2H), | |
| | | 2.73 (s, 3H), 2.46 (s, 3H), 2.38-2.33 | |
| | | (m, 1H), 2.09-2.02 (m, 1H), 1.99 (s, | |
| | | 3H). | |
| 300 | 728.3 | ¹H NMR (500 MHz, DMSO-d6) δ | 5 |
| | | 12.44 (s, 1H), 10.82 (s, 1H), 8.23 (d, | |
| | | J = 2.1 Hz, 1H), 7.92-7.91 (m, 1H), | |
| | | 7.82 (d, J = 2.2 Hz, 1H), 7.79-7.73 | |
| | | (m, 2H), 7.62-7.58 (m, 1H), 7.57- | |
| | | 7.56 (m, 1H), 7.42 (d, J = 8.8 Hz, | |
| | | 1H), 7.20-7.16 (m, 1H), 7.04-6.98 | |
| | | (m, 2H), 6.11-6.07 (m, 1H), 4.39- | |
| | | 4.21 (m, 2H), 4.19-3.90 (m, 4H), | |
| | | 3.76-3.69 (m, 1H), 3.35-3.28 (m, | |
| | | 2H), 3.24-2.85 (m, 4H), 2.43 (s, | |
| | | 3H), 2.41-2.20 (m, 2H), 1.82 (s, | |
| | | 3H). | |
| 301 | 760.3 | ¹H NMR (500 MHz, DMSO-d₆) δ | 5 |
| | | 10.01 (s, 1H), 9.93 (s, 1H), 8.23 (d, J = | |
| | | 2.2 Hz, 1H), 7.92-7.89 (m, 1H), | |
| | | 7.65 (d, J = 2.1 Hz, 1H), 7.62-7.59 | |
| | | (m, 1H), 7.55 (d, J = 2.0 Hz, 1H), | |
| | | 7.47-7.40 (m, 2H), 7.25-7.24 (m, | |
| | | 1H), 7.20-7.16 (m, 1H), 7.05-6.98 | |
| | | (m, 2H), 6.11-6.08 (m, 1H), 4.39- | |
| | | 4.08 (m, 3H), 3.90-3.67 (m, 4H), | |
| | | 3.35-2.72 (m, 6H), 2.38-2.33 (m, | |
| | | 1H), 2.22 (s, 3H), 2.19-1.88 (m, | |
| | | 8H). | |
| 302 | 767.2 | ¹H NMR (500 MHz, DMSO-d₆) δ | 5 |
| | | 8.23 (d, J = 2.1 Hz, 1H), 7.92-7.91 | |
| | | (m, 1H), 7.87-7.82 (m, 2H), 7.62- | |
| | | 7.59 (m, 1H), 7.47-7.40 (m, 2H), | |
| | | 7.25-6.98 (m, 4H), 6.11-6.08 (m, | |
| | | 1H), 4.37-4.33 (m 1H), 4.32-4.28 | |
| | | (m, 1H), 4.08-3.80 (m, 3H), 3.80- | |
| | | 3.67 (m, 2H), 3.35-3.30 (m, 1H), | |
| | | 2.89-2.81 (m, 2H), 2.80-2.72 (m, | |
| | | 2H), 2.44 (s, 3H), 2.38-2.33 (m, | |
| | | 1H), 2.09-2.02 (m, 1H), 1.99 (s, | |
| | | 3H), 0.93 (d, J = 8.4 Hz, 3H). | |
| 303 | 735.2 | ¹H NMR (500 MHz, DMSO-d₆) δ | 5 |
| | | 10.64 (s, 1H), 10.53 (s, 1H), 9.97 (s, | |
| | | 1H), 8.23 (d, J = 2.2 Hz, 1H), 8.16 (d, | |
| | | J = 2.1 Hz, 1H), 7.92-7.89 (m, 1H), | |
| | | 7.62-7.59 (m, 1H), 7.47-7.40 (m, | |
| | | 3H), 7.25-7.24 (m, 1H), 7.20-7.16 | |
| | | (m, 1H), 7.05-6.98 (m, 2H), 6.11- | |
| | | 6.08 (m, 1H), 4.39-4.08 (m, 3H), | |
| | | 3.90-3.67 (m, 4H), 3.35-2.73 (m, | |
| | | 6H), 2.38-2.33 (m, 1H), 2.28 (s, | |
| | | 3H), 2.09-2.02 (m, 1H), 1.99 (s, | |
| | | 3H). | |
| 304 | 737.3 | ¹H NMR (500 MHz, DMSO-d₆) δ | 5 |
| | | 12.40 (s, 1H), 10.64 (s, 1H), 8.24 (d, | |
| | | J = 2.2 Hz, 1H), 7.97 (d, J = 1.8 Hz, | |
| | | 1H), 7.80-7.77 (m, 1H), 7.62-7.54 | |
| | | (m, 2H), 7.49-7.45 (m, 2H), 7.25- | |
| | | 7.24 (m, 1H), 7.20-7.16 (m, 1H), | |
| | | 7.05-6.98 (m, 2H), 6.11-6.08 (m, | |
| | | 1H), 4.39-3.68 (m, 7H), 3.35-2.72 | |
| | | (m, 6H), 2.42 (s, 3H), 2.32-2.14 (m, | |
| | | 2H), 1.99 (s, 3H). | |
| 305 | 733.3 | ¹H NMR (500 MHz, DMSO-d₆) δ | 5 |
| | | 12.38 (s, 1H), 10.76 (s, 1H), 8.32 (d, | |
| | | J = 2.1 Hz, 1H), 7.95 (d, J = 1.8 Hz, | |
| | | 1H), 7.83 (d, J = 2.1 Hz, 1H), 7.62- | |
| | | 7.56 (m, 2H), 7.49-7.45 (m, 2H), | |
| | | 7.25-7.24 (m, 1H), 7.20-7.16 (m, | |
| | | 1H), 7.05-6.98 (m, 2H), 6.11-6.08 | |
| | | (m, 1H), 4.39-3.68 (m, 7H), 3.35- | |
| | | 2.72 (m, 6H), 2.70 (s, 3H), 2.44 (s, | |
| | | 3H), 2.32-2.14 (m, 2H), 1.99 (s, 3H). | |
| 306 | 719.3 | ¹H NMR (500 MHz, DMSO-d₆) δ | 5 |
| | | 12.60 (s, 1H), 10.50 (s, 1H), 8.42 (d, | |
| | | J = 1.8 Hz, 1H), 7.97 (s, 1H), 7.91- | |
| | | 7.90 (m, 1H), 7.70 (d, J = 7.9 Hz, | |
| | | 1H), 7.62-7.56 (m, 2H), 7.49- | |
| | | 7.45 (m, 2H), 7.25-7.24 (m, 1H), | |
| | | 7.20-7.16 (m, 1H), 7.05-6.98 (m, | |
| | | 2H), 6.11-6.08 (m, 1H), 4.39-3.68 | |
| | | (m, 7H), 3.35-2.37 (m, 6H), 2.43 (s, | |
| | | 3H), 2.32-2.14 (m, 2H), 1.99 (s, 3H). | |
| 307 | 760.3 | ¹H NMR (500 MHz, DMSO-d₆) δ | 5 |
| | | 10.31 (s, 1H), 9.93 (s, 1H), 8.23 (d, J = | |
| | | 2.2 Hz, 1H), 7.92-7.89 (m, 1H), | |
| | | 7.65 (d, J = 2.1 Hz, 1H), 7.62-7.59 | |
| | | (m, 1H), 7.55 (d, J = 2.0 Hz, 1H), | |
| | | 7.47-7.40 (m, 2H), 7.25-7.24 (m, | |
| | | 1H), 7.20-7.16 (m, 1H), 7.05-6.98 | |
| | | (m, 2H), 6.11-6.08 (m, 1H), 4.39- | |
| | | 4.08 (m, 3H), 3.90-3.67 (m, 4H), | |
| | | 3.35-2.72 (m, 6H), 2.38-2.33 (m, | |
| | | 1H), 2.22 (s, 3H), 2.18-1.72 (m, | |
| | | 8H). | |
| 308 | 753.2 | 1H NMR (500 MHz, DMSO-d6) δ | 5 |
| | | 12.40 (s, 1H), 10.85 (s, 1H), 8.23 (d, | |
| | | J = 2.1 Hz, 1H), 7.92-7.91 (m, 1H), | |
| | | 7.87-7.82 (m, 2H), 7.62-7.59 (m, | |
| | | 1H), 7.47-7.40 (m, 2H), 7.25-7.24 | |
| | | (m, 1H), 7.20-7.16 (m, 1H), 7.05- | |
| | | 6.98 (m, 2H), 6.11-6.08 (m, 1H), | |
| | | 4.39-4.21 (m, 2H), 4.07-4.03 (m, | |
| | | 1H), 3.85-3.77 (m, 4H), 3.35-2.72 | |
| | | (m, 6H), 2.44 (s, 3H), 2.32-2.14 (m, | |
| | | 2H), 1.99 (s, 3H). | |
| 309 | 735.2 | ¹H NMR (500 MHz, DMSO-d₆) δ | 5 |
| | | 10.64 (s, 1H), 10.53 (s, 1H), 9.97 (s, | |
| | | 1H), 8.23 (d, J = 2.2 Hz, 1H), 8.16 (d, | |
| | | J = 2.1 Hz, 1H), 7.92-7.89 (m, 1H), | |
| | | 7.62-7.59 (m, 1H), 7.47-7.40 (m, | |
| | | 3H), 7.25-7.24 (m, 1H), 7.20-7.16 | |
| | | (m, 1H), 7.05-6.98 (m, 2H), 6.11- | |
| | | 6.08 (m, 1H), 4.39-4.08 (m, 3H), | |
| | | 3.90-3.67 (m, 4H), 3.35-2.73 (m, | |
| | | 6H), 2.38-2.33 (m, 1H), 2.28 (s, | |
| | | 3H), 2.09-2.02 (m, 1H), 1.99 (s, | |
| | | 3H). | |
| 310 | 724.3 | ¹H NMR (500 MHz, DMSO-d₆) δ | 5 |
| | | 12.55 (s, 1H), 10.52 (s, 1H), 8.43 (d, | |
| | | J = 1.3 Hz, 1H), 7.97 (s, 1H), 7.92- | |
| | | 7.91 (m, 1H), 7.79-7.73 (m, 2H), | |
| | | 7.72 (d, J = 5.2 Hz, 1H), 7.63-7.53 | |
| | | (m, 2H), 7.46 (d, J = 8.8 Hz, 1H), | |
| | | 7.20-7.16 (m, 1H), 7.05-6.98 (m, | |
| | | 2H), 6.11-6.08 (m, 1H), 4.39-3.68 | |
| | | (m, 7H), 3.35-3.31 (m, 1H), 3.31- | |
| | | 3.27 (m, 1H), 2.89-2.73 (m, 6H), | |
| | | 2.32-2.14 (m, 2H), 1.99 (s, 3H), | |
| | | 1.33-1.14 (m, 3H). | |
| 311 | 738.3 | ¹H NMR (500 MHz, DMSO-d₆) δ | 5 |
| | | 12.49 (s, 1H), 10.32 (s, 1H), 8.53 (d, | |
| | | J = 2.0 Hz, 1H), 7.97 (s, 1H), 7.92- | |
| | | 7.91 (m, 1H), 7.80-7.72 (m, 3H), | |
| | | 7.63-7.53 (m, 2H), 7.46 (d, J = 8.8 | |
| | | Hz, 1H), 7.20-7.16 (m, 1H), 7.05- | |
| | | 6.98 (m, 2H), 6.11-6.08 (m, 1H), | |
| | | 4.39-3.68 (m, 7H), 3.35-3.27 (m, | |
| | | 2H), 3.17-3.07 (m, 1H), 2.89-2.73 | |
| | | (m, 4H), 2.32-2.14 (m, 2H), 1.99 (s, | |
| | | 3H), 1.65 (d, J = 6.5 Hz, 3H), 1.60 (d, | |
| | | J = 6.5 Hz, 3H). | |
| 312 | 736.3 | ¹H NMR (500 MHz, DMSO-d₆) δ | 5 |
| | | 12.61 (s, 1H), 10.32 (s, 1H), 8.49 (d, | |
| | | J = 1.8 Hz, 1H), 7.97 (d, J = 1.8 Hz, | |
| | | 1H), 7.92-7.91 (m, 1H), 7.80-7.73 | |
| | | (m, 2H), 7.76-7.73 (m, 1H), 7.63- | |
| | | 7.53 (m, 2H), 7.46 (d, J = 8.8 Hz, | |
| | | 1H), 7.20-7.16 (m, 1H), 7.05-6.98 | |
| | | (m, 2H), 6.11-6.08 (m, 1H), 4.39- | |
| | | 3.68 (m, 7H), 3.35-3.72 (m, 6H), | |
| | | 2.34-2.14 (m, 3H), 1.99 (s, 3H), | |
| | | 1.13-0.89 (m, 4H). | |
| 313 | 787.2 | ¹H NMR (500 MHz, DMSO-d₆) δ | 5 |
| | | 12.75 (s, 1H), 10.78 (s, 1H), 8.36 (d, | |
| | | J = 2.1 Hz, 1H), 8.27 (d, J = 2.1 Hz, | |
| | | 1H), 7.97 (d, J = 2.1 Hz, 1H), 7.62- | |
| | | 7.56 (m, 2H), 7.49-7.54 (m, 2H), | |
| | | 7.25-7.24 (m, 1H), 7.20-7.16 (m, | |
| | | 1H), 7.05-6.98 (m, 2H), 6.11-6.08 | |
| | | (m, 1H), 5.11-4.79 (m, 1H), 4.34- | |
| | | 3.98 (m, 2H), 3.90-3.67 (m, 4H), | |
| | | 3.35-2.72 (m, 6H), 2.45 (s, 3H), | |
| | | 2.36-2.03 (m, 2H), 1.99 (s, 3H). | |
| 314 | 764.2 | ¹H NMR (500 MHz, DMSO-d₆) δ | 5 |
| | | 10.50 (s, 1H), 10.03 (s, 1H), 7.99- | |
| | | 7.94 (m, 2H), 7.84-7.81 (m, 1H), | |
| | | 7.62-7.54 (m, 2H), 7.49-7.45 (m, | |
| | | 2H), 7.25-7.24 (m, 1H), 7.20-7.16 | |
| | | (m, 1H), 7.05-6.98 (m, 2H), 6.11- | |
| | | 6.08 (m, 1H), 5.11-4.79 (m, 1H), | |
| | | 4.34-3.98 (m, 2H), 3.90-3.67 (m, | |
| | | 4H), 3.35-2.72 (m, 6H), 2.36-2.03 | |
| | | (m, 4H), 1.99 (s, 3H), 1.71-1.60 (m, | |
| | | 2H). | |
| 315 | 745.3 | ¹H NMR (500 MHz, DMSO-d₆) δ | 5 |
| | | 13.27 (s, 1H), 10.59 (s, 1H), 8.56 (d, | |
| | | J = 1.8 Hz, 1H), 7.97 (d, J = 1.8 Hz, | |
| | | 1H), 7.95-7.90 (m, 1H), 7.65 (d, J = | |
| | | 7.9 Hz, 1H), 7.62-7.56 (m, 2H), | |
| | | 7.49-7.45 (m, 2H), 7.25-7.24 (m, | |
| | | 1H), 7.20-7.16 (m, 1H), 7.05-6.98 | |
| | | (m, 2H), 6.11-6.08 (m, 1H), 5.38- | |
| | | 5.34 (m, 1H), 5.32-5.28 (m, 1H), | |
| | | 4.39-4.21 (m, 2H), 4.04-3.97 (m, | |
| | | 1H), 3.85-3.77 (m, 4H), 3.35-2.72 | |
| | | (m, 6H), 2.32 (s, 3H), 2.31-2.23 (m, | |
| | | 1H), 2.20-1.14 (m, 1H), 1.99 (s, | |
| | | 3H). | |
| 316 | 759.2 | ¹H NMR (500 MHz, DMSO-d₆) δ | 5 |
| | | 12.40 (s, 1H), 10.64 (s, 1H), 8.24 (d, | |
| | | J = 2.2 Hz, 1H), 7.97 (d, J = 1.8 Hz, | |
| | | 1H), 7.80-7.73 (m, 2H), 7.76-7.73 | |
| | | (m, 1H), 7.67-7.65 (m, 1H), 7.63- | |
| | | 7.54 (m, 2H), 7.48 (d, J = 8.0 Hz, | |
| | | 1H), 7.20 (d, J = 0.8 Hz, 1H), 7.20- | |
| | | 7.16 (m, 2H), 7.08-7.02 (m, 1H), | |
| | | 4.38-4.18 (m, 4H), 4.01-3.94 (m, | |
| | | 1H), 3.88-3.68 (m, 2H), 2.42 (s, | |
| | | 3H), 2.32-2.14 (m, 2H), 1.99 (s, | |
| | | 3H). | |
| 317 | 737.3 | ¹H NMR (500 MHz, DMSO-d₆) δ | 5 |
| | | 12.05 (s, 1H), 10.46 (s, 1H), 8.32 (d, | |
| | | J = 2.1 Hz, 1H), 7.97 (d, J = 1.8 Hz, | |
| | | 1H), 7.80 (d, J = 2.0 Hz, 1H), 7.67- | |
| | | 7.74 (m, 2H), 7.69-7.65 (m, 1H), | |
| | | 7.63-7.53 (m, 2H), 7.48 (d, J = 8.0 | |
| | | Hz, 1H), 7.45-7.41 (m, 2H), 7.35- | |
| | | 7.29 (m, 1H), 7.20-7.16 (m, 1H), | |
| | | 7.08-7.05 (m, 1H), 4.38-4.30 (m, | |
| | | 1H), 4.24-4.16 (m, 2H), 4.15- | |
| | | 4.07 (m, 2H), 3.88-3.68 (m, 2H), | |
| | | 2.72 (s, 3H), 2.45 (s, 3H), 2.32-2.14 | |
| | | (m, 2H), 1.99 (s, 3H). | |
| 318 | 750.2 | ¹H NMR (500 MHz, DMSO-d₆) δ | 5 |
| | | 12.60 (s, 1H), 10.50 (s, 1H), 8.42 (d, | |
| | | J = 1.8 Hz, 1H), 7.97 (s, 1H), 7.91- | |
| | | 7.90 (m, 1H), 7.70 (d, J = 7.9 Hz, | |
| | | 1H), 7.62-7.56 (m, 2H), 7.53-7.42 | |
| | | (m, 3H), 7.42-7.34 (m, 2H), 7.25- | |
| | | 7.24 (m, 1H), 7.18-7.17 (m, 1H), | |
| | | 7.08-7.02 (m, 1H), 4.38-4.30 (m, | |
| | | 1H), 4.24-3.94 (m, 4H), 3.88-3.68 | |
| | | (m, 2H), 2.43 (s, 3H), 2.32-2.14 (m, | |
| | | 2H), 1.99 (s, 3H). | |
| 319 | 791.2 | ¹H NMR (500 MHz, DMSO-d₆) δ | 5 |
| | | 10.34 (s, 1H), 9.93 (s, 1H), 7.99- | |
| | | 7.93 (m, 2H), 7.88 (d, J = 2.1 Hz, | |
| | | 1H), 7.67-7.65 (m, 1H), 7.62-7.56 | |
| | | (m, 2H), 7.51-7.42 (m, 2H), 7.25- | |
| | | 7.23 (m, 2H), 7.20-7.16 (m, 2H), | |
| | | 7.08-7.02 (m, 1H), 4.38-4.30 (m, | |
| | | 4H), 4.01-3.77 (m, 3H), 2.38-2.33 | |
| | | (m, 1H), 2.21 (s, 3H), 2.20-2.00 (m, | |
| | | 3H), 1.99 (s, 3H), 1.72-1.65 (m, | |
| | | 1H), 1.67-1.61 (m, 1H). | |
| 320 | 784.2 | ¹H NMR (500 MHz, DMSO-d₆) δ | 5 |
| | | 12.43 (s, 1H), 10.60 (s, 1H), 8.30 (d, | |
| | | J = 2.1 Hz, 1H), 8.01 (d, J = 2.1 Hz, | |
| | | 1H), 7.97 (s, 1H), 7.62-7.56 (m, | |
| | | 2H), 7.53-7.45 (m, 2H), 7.47-7.42 | |
| | | (m, 1H), 7.42-7.34 (m, 2H), 7.25- | |
| | | 7.24 (m, 1H), 7.18-7.17 (m, 1H), | |
| | | 7.08-7.02 (m, 1H), 4.38-4.30 (m, | |
| | | 1H), 4.24-3.94 (m, 4H), 3.88-3.68 | |
| | | (m, 2H), 2.45 (s, 3H), 2.32-2.14 (m, | |
| | | 2H), 1.99 (s, 3H). | |
| 321 | 766.2 | ¹H NMR (500 MHz, DMSO-d₆) δ | 5 |
| | | 10.66 (s, 1H), 10.53 (s, 1H), 10.23 (s, | |
| | | 1H), 8.25 (d, J = 2.1 Hz, 1H), 7.97 (d, | |
| | | J = 2.0 Hz, 1H), 7.83 (d, J =2.4 Hz, | |
| | | 1H), 7.67-7.65 (m, 1H), 7.62-7.56 | |
| | | (m, 2H), 7.51-7.42 (m, 2H), 7.25- | |
| | | 7.23 (m, 2H), 7.20-7.16 (m, 2H), | |
| | | 7.08-7.02 (m, 1H), 4.38-4.30 (m, | |
| | | 4H), 4.01-3.77 (m, 3H), 2.36-2.31 | |
| | | (m, 1H), 2.30 (s, 3H), 2.09-2.02 (m, | |
| | | 1H), 1.99 (s, 3H). | |
| 322 | 720.2 | ¹H NMR (500 MHz, DMSO-d₆) δ | 5 |
| | | 12.60 (s, 1H), 10.50 (s, 1H), 8.42 (d | |
| | | J = 1.8 Hz, 1H), 7.97 (d, J = 2.0 Hz, | |
| | | 1H), 7.91-7.90 (m, 1H), 7.70 (d, J = | |
| | | 7.9 Hz, 1H), 7.68-7.66 (m, 1H), | |
| | | 7.62-7.56 (m, 2H), 7.51-7.42 (m, | |
| | | 2H), 7.25-7.24 (m, 1H), 7.17-7.15 | |
| | | (m, 1H), 7.11-6.98 (m, 3H), 4.38- | |
| | | 3.97 (m, 5H), 3.87-3.73 (m, 2H), | |
| | | 2.43 (s, 3H), 2.19-2.10 (m, 2H), | |
| | | 1.98 (s, 3H). | |
| 323 | 769.3 | ¹H NMR (500 MHz, DMSO-d₆) δ | 5 |
| | | 12.49 (s, 1H), 10.32 (s, 1H), 8.53 (d, | |
| | | J = 1.8 Hz, 1H), 7.97 (s, 1H), 7.92- | |
| | | 7.91 (m, 1H), 7.80-7.72 (m, 3H), | |
| | | 7.63-7.53 (m, 2H), 7.53-7.45 (m, | |
| | | 2H), 7.42-7.34 (m, 2H), 7.18- | |
| | | 7.17 (m, 1H), 7.08-7.04 (m, 1H), | |
| | | 4.38-4.30 (m, 1H), 4.24-3.94 (m, | |
| | | 4H), 3.88-3.17 (m, 3H), 2.32-2.14 | |
| | | (m, 2H), 1.99 (s, 3H), 1.65 (d, J = 6.5 | |
| | | Hz, 3H), 1.60 (d, J = 6.5 Hz, 3H). | |
| 324 | 772.2 | ¹H NMR (500 MHz, DMSO-d₆) δ | 5 |
| | | 12.61 (s, 1H), 10.32 (s, 1H), 8.49 (d, | |
| | | J = 1.8 Hz, 1H), 7.97 (d, J = 1.8 Hz, | |
| | | 1H), 7.92-7.91 (m, 1H), 7.77 (d, J = | |
| | | 7.9 Hz, 1H), 7.70-7.66 (m, 1H), | |
| | | 7.62-7.56 (m, 2H), 7.51-7.42 (m, | |
| | | 2H), 7.25-7.23 (m, 1H), 7.24-7.21 | |
| | | (m, 1H), 7.20 (d, J = 2.1 Hz, 1H), | |
| | | 7.19-7.11 (m, 2H), 4.38-4.20 (m, | |
| | | 2H), 4.17-3.68 (m, 5H), 2.35-2.23 | |
| | | (m, 5H), 2.20-2.14 (m, 1H), 1.99 (s, | |
| | | 3H), 1.03-0.89 (m, 4H). | |
| 325 | 791.2 | ¹H NMR (500 MHz, DMSO-d₆) δ | 5 |
| | | 12.75 (s, 1H), 10.78 (s, 1H), 8.36 (d, | |
| | | J = 2.1 Hz, 1H), 8.27 (d, J = 2.1 Hz, | |
| | | 1H), 7.90 (d, J = 2.0 Hz, 1H), 7.79- | |
| | | 7.73 (m, 2H), 7.69-7.65 (m, 1H), | |
| | | 7.63-7.53 (m, 2H), 7.48 (d, J = 8.0 | |
| | | Hz, 1H), 7.45-7.41 (m, 2H), 7.35- | |
| | | 7.29 (m, 1H), 7.20-7.16 (m, 1H), | |
| | | 7.08-7.05 (m, 1H), 4.38-4.28 (m, | |
| | | 2H), 4.21-4.16 (m, 1H), 4.15- | |
| | | 4.07 (m, 2H), 3.90-3.67 (m, 2H), | |
| | | 2.45 (s, 3H), 2.36-2.03 (m, 2H), | |
| | | 1.99 (s, 3H). | |
| 326 | 795.2 | ¹H NMR (500 MHz, DMSO-d₆) δ | 5 |
| | | 10.50 (s, 1H), 10.03 (s, 1H), 7.99- | |
| | | 7.94 (m, 2H), 7.84-7.81 (m, 1H), | |
| | | 7.67-7.65 (m, 1H), 7.62-7.54 (m, | |
| | | 2H), 7.51-7.42 (m, 2H), 7.25-7.23 | |
| | | (m, 2H), 7.20-7.16 (m, 2H), 7.08- | |
| | | 7.02 (m, 1H), 4.38-4.30 (m, 4H), | |
| | | 4.01-3.77 (m, 3H), 2.38-2.33 (m, | |
| | | 1H), 2.18-1.65 (m, 4H), 1.19-0.89 | |
| | | (m, 4H) | |
| 327 | 776.2 | ¹H NMR (500 MHz, DMSO-d₆) δ | 5 |
| | | 13.27 (s, 1H), 10.59 (s, 1H), 8.56 (d, | |
| | | J = 1.8 Hz, 1H), 7.97 (d, J = 2.0 Hz, | |
| | | 1H), 7.95-7.90 (m, 1H), 7.68-7.66 | |
| | | (m, 2H), 7.62-7.56 (m, 2H), 7.51- | |
| | | 7.42 (m, 2H), 7.25-7.23 (m, 2H), | |
| | | 7.20-7.16 (m, 2H), 7.08-7.02 (m, | |
| | | 1H), 5.38-5.33 (m, 1H), 5.33-5.28 | |
| | | (m, 1H), 4.38-4.18 (m, 4H), 4.00- | |
| | | 3.93 (m, 1H), 3.88-3.68 (m, 2H), | |
| | | 2.34-2.28 (m, 3H), 2.31-2.23 (m, | |
| | | 1H), 2.20-2.14 (m, 1H), 2.03 (s, | |
| | | 3H). | |
| 328 | 723.2 | ¹H NMR (500 MHz, DMSO-d₆) δ | 5 |
| | | 8.22-8.14 (m, 2H), 7.92-7.89 (m, | |
| | | 1H), 7.70-7.62 (m, 2H), 7.62-7.59 | |
| | | (m, 1H), 7.47-7.40 (m, 2H), 7.25- | |
| | | 7.24 (m, 1H), 7.20-7.16 (m, 1H), | |
| | | 7.05-6.98 (m, 2H), 6.11-6.08 (m, | |
| | | 1H), 4.39-4.21 (m, 2H), 4.07-4.03 | |
| | | (m, 1H), 3.85-3.77 (m, 4H), 3.35- | |
| | | 2.72 (m, 6H), 2.32-2.14 (m, 2H), | |
| | | 2.01 (s, 3H). | |
| 329 | 767.2 | ¹H NMR (500 MHz, DMSO-d₆) δ | |
| | | 8.23 (d, J = 2.1 Hz, 1H), 7.92-7.91 | |
| | | (m, 1H), 7.87-7.82 (m, 2H), 7.62- | |
| | | 7.59 (m, 1H), 7.47-7.40 (m, 2H), | |
| | | 7.25-6.98 (m, 4H), 6.11-6.08 (m, | |
| | | 1H), 4.37-4.33 (m, 1H), 4.32-4.28 | |
| | | (m, 1H), 4.08-3.80 (m, 3H), 3.80- | |
| | | 3.67 (m, 2H), 3.35-3.30 (m, 1H), | |
| | | 2.89-2.81 (m, 2H), 2.80-2.72 (m, | |
| | | 2H), 2.44 (s, 3H), 2.38-2.33 (m, | |
| | | 1H), 2.09-2.02 (m, 1H), 1.98 (s, | |
| | | 3H), 0.93 (d, J = 8.4 Hz, 3H). | |
| 330 | 745.3 | ¹H NMR (500 MHz, DMSO-d₆) δ | 5 |
| | | 12.64 (s, 1H), 10.83 (s, 1H), 8.23 (d, | |
| | | J = 2.0 Hz, 2H), 8.13 (d, J = 2.0 Hz, | |
| | | 1H), 7.92-7.89 (m, 1H), 7.70-7.66 | |
| | | (m, 1H), 7.62-7.55 (m, 2H), 7.47- | |
| | | 7.40 (m, 2H), 7.25-7.24 (m, 1H), | |
| | | 7.20-7.16 (m, 1H), 7.05-6.98 (m, | |
| | | 2H), 6.11-6.08 (m, 1H), 4.39-4.21 | |
| | | (m, 2H), 4.07-4.03 (m, 1H), 3.85- | |
| | | 3.77 (m, 4H), 3.35-3.27 (m, 2H), | |
| | | 2.89-2.81 (m, 2H), 2.80-2.72 (m, | |
| | | 2H), 2.34-2.14 (m, 3H), 1.99 (s, | |
| | | 3H), 1.14-0.81 (m, 4H). | |
| 331 | 789.3 | ¹H NMR (500 MHz, DMSO-d₆) δ | 5 |
| | | 12.71 (s, 1H), 10.84 (s, 1H), 8.23 (d, | |
| | | J = 2.1 Hz, 1H), 8.08 (d, J = 2.1 Hz, | |
| | | 1H), 7.92-7.91 (m, 1H), 7.86 (d, J = | |
| | | 2.1 Hz, 1H), 7.62-7.59 (m, 1H), | |
| | | 7.47-7.40 (m, 2H), 7.25-7.24 (m, | |
| | | 1H), 7.02-6.93 (m, 3H), 4.39-4.21 | |
| | | (m, 2H), 4.07-4.03 (m, 1H), 3.88- | |
| | | 3.68 (m, 2H), 3.69 (s, 2H), 2.97- | |
| | | 2.90 (m, 1H), 2.87-2.68 (m, 4H), | |
| | | 2.46 (s, 3H), 2.32-2.14 (m, 2H), | |
| | | 2.02-1.85 (m, 7H). | |
| 332 | 789.3 | ¹H NMR (500 MHz, DMSO-d₆) δ | 5 |
| | | 12.71 (s, 1H), 10.84 (s, 1H), 8.23 (d, | |
| | | J = 2.1 Hz, 1H), 8.08 (d, J = 2.1 Hz, | |
| | | 1H), 7.92-7.91 (m, 1H), 7.86 (d, J = | |
| | | 2.1 Hz, 1H), 7.62-7.59 (m, 1H), | |
| | | 7.47-7.40 (m, 2H), 7.25-7.24 (m, | |
| | | 1H), 7.02-6.93 (m, 3H), 4.39-4.21 | |
| | | (m, 2H), 4.07-4.03 (m, 1H), 3.88- | |
| | | 3.68 (m, 2H), 3.69 (s, 2H), 2.97- | |
| | | 2.90 (m, 1H), 2.87-2.69 (m, 4H), | |
| | | 2.46 (s, 3H), 2.32-2.14 (m, 2H), | |
| | | 2.04-1.87 (m, 7H). | |
| 333 | 775.2 | ¹H NMR (500 MHz, DMSO-d₆) δ | 5 |
| | | 13.03 (s, 1H), 10.94 (s, 1H), 8.23 (d, | |
| | | J = 2.1 Hz, 1H), 8.12 (d, J = 2.1 Hz, | |
| | | 1H), 7.92-7.89 (m, 1H), 7.67-7.65 | |
| | | (m, 1H), 7.63-7.56 (m, 2H), 7.47- | |
| | | 7.40 (m, 2H), 7.25-7.24 (m, 1H), | |
| | | 7.02-6.93 (m, 3H), 4.39-4.21 (m, | |
| | | 2H), 4.07-4.03 (m, 1H), 3.88-3.68 | |
| | | (m, 2H), 3.69 (s, 2H), 2.97-2.90 (m, | |
| | | 1H), 2.87-2.69 (m, 4H), 2.32-2.14 | |
| | | (m, 2H), 2.02-1.87 (m, 7H). | |
| 334 | 730.3 | ¹H NMR (500 MHz, DMSO-d₆) δ | 5 |
| | | 12.44 (s, 1H), 10.82 (s, 1H), 8.23 (d, | |
| | | J = 2.1 Hz, 1H), 7.92-7.89 (m, 1H), | |
| | | 7.82 (d, J = 2.2 Hz, 1H), 7.79-7.73 | |
| | | (m, 2H), 7.63-7.59 (m, 1H), 7.57- | |
| | | 7.52 (m, 1H), 7.42 (d, J = 8.8 Hz, | |
| | | 1H), 7.02-6.93 (m, 3H), 4.39-4.21 | |
| | | (m, 2H), 4.07-4.03 (m, 1H), 3.88- | |
| | | 3.68 (m, 2H), 3.69 (s, 2H), 2.97- | |
| | | 2.90 (m, 1H), 2.87-2.69 (m, 4H), | |
| | | 2.43 (s, 3H), 2.32-2.14 (m, 2H), | |
| | | 2.05-1.85 (m, 7H). | |
| 335 | 726.3 | ¹H NMR (500 MHz, DMSO-d₆) δ | 5 |
| | | 12.00 (s, 1H), 10.69 (s, 1H), 8.23 (d, | |
| | | J = 2.1 Hz, 1H), 7.92-7.89 (m, 1H), | |
| | | 7.78-7.73 (m, 1H), 7.70 (d, J = 2.1 | |
| | | Hz, 1H), 7.47-7.41 (m, 2H), 7.02- | |
| | | 6.90 (m, 5H), 4.39-4.21 (m, 2H), | |
| | | 4.07-4.03 (m, 1H), 3.88-3.68 (m, | |
| | | 2H), 3.69 (s, 2H), 3.23-2.89 (m, | |
| | | 3H), 2.73 (s, 3H), 2.69-2.61 (m, | |
| | | 2H), 2.46 (s, 3H), 2.32-2.14 (m, | |
| | | 2H), 2.02-1.85 (m, 7H). | |
| 336 | 721.3 | ¹H NMR (500 MHz, DMSO-d₆) δ | 5 |
| | | 12.67 (s, 1H), 10.56 (s, 1H), 8.23 (d, | |
| | | J = 2.1 Hz, 1H), 7.92-7.89 (m, 2H), | |
| | | 7.68-7.66 (m, 1H), 7.62-7.59 (m, | |
| | | 1H), 7.49-7.40 (m, 3H), 7.25-7.24 | |
| | | (m, 1H), 7.02-6.93 (m, 3H), 4.39- | |
| | | 4.21 (m, 2H), 4.07-4.03 (m, 1H), | |
| | | 3.88-3.68 (m, 2H), 3.69 (s, 2H), | |
| | | 2.97-2.90 (m, 1H), 2.87-2.79 (m, | |
| | | 5H), 2.69-2.61 (m, 2H), 2.32-2.14 | |
| | | (m, 2H), 2.07-1.84 (m, 7H). | |
| 337 | 762.3 | ¹H NMR (500 MHz, DMSO-d₆) δ | 5 |
| | | 10.01 (s, 1H), 9.93 (s, 1H), 8.23 (d, J = | |
| | | 2.2 Hz, 1H), 7.92-7.89 (m, 1H), | |
| | | 7.65 (d, J = 2.1 Hz, 1H), 7.62-7.59 | |
| | | (m, 1H), 7.55 (d, J = 2.0 Hz, 1H), | |
| | | 7.47-7.40 (m, 2H), 7.25-7.24 (m, | |
| | | 1H), 7.02-6.93 (m, 3H), 4.39- | |
| | | 4.08 (m, 3H), 3.90-3.67 (m, 4H), | |
| | | 2.97-2.90 (m, 1H), 2.87-2.69 (m, | |
| | | 4H), 2.38-2.33 (m, 1H), 2.22 (s, | |
| | | 3H), 2.17-2.08 (m, 2H), 2.08-2.01 | |
| | | (m, 1H), 2.07-1.93 (m, 7H), 1.72- | |
| | | 1.61 (m, 2H). | |
| 338 | 755.2 | ¹H NMR (500 MHz, DMSO-d₆) δ | 5 |
| | | 12.40 (s, 1H), 10.75 (s, 1H), 8.23 (d, | |
| | | J = 2.1 Hz, 1H), 7.92-7.91 (m, 1H), | |
| | | 7.87-7.82 (m, 2H), 7.62-7.59 (m, | |
| | | 1H), 7.47-7.40 (m, 2H), 7.25-7.24 | |
| | | (m, 1H), 7.02-6.93 (m, 3H), 4.39- | |
| | | 4.21 (m, 2H), 4.07-4.03 (m, 1H), | |
| | | 3.88-3.68 (m, 2H), 3.69 (s, 2H), | |
| | | 2.97-2.90 (m, 1H), 2.87-2.69 (m, | |
| | | 4H), 2.44 (s, 3H), 2.32-2.14 (m, | |
| | | 2H), 2.04-1.87 (m, 7H). | |
| 339 | 737.3 | ¹H NMR (500 MHz, DMSO-d₆) δ | 5 |
| | | 10.64 (s, 1H), 10.53 (s, 1H), 9.97 (s, | |
| | | 1H), 8.23 (d, J = 2.1 Hz, 1H), 8.16 (d, | |
| | | J = 2.1 Hz, 1H), 7.92-7.89 (m, 1H), | |
| | | 7.62-7.59 (m, 1H), 7.47-7.40 (m, | |
| | | 2H), 7.42 (d, J = 1.3 Hz, 1H), 7.25- | |
| | | 7.24 (m, 1H), 7.02-6.93 (m, 3H), | |
| | | 4.39-4.08 (m, 3H), 3.90-3.67 (m, | |
| | | 2H), 3.69 (s, 2H), 2.97-2.90 (m, | |
| | | 1H), 2.87-2.69 (m, 4H), 2.38-2.33 | |
| | | (m, 1H), 2.28 (s, 3H), 2.09-1.85 (m, | |
| | | 8H). | |
| 340 | 739.2 | ¹H NMR (500 MHz, DMSO-d₆) δ | 5 |
| | | 12.44 (s, 1H), 10.82 (s, 1H), 8.23 (d, | |
| | | J = 2.1 Hz, 1H), 7.92-7.89 (m, 1H), | |
| | | 7.82 (d, J = 2.2 Hz, 1H), 7.79-7.73 | |
| | | (m, 2H), 7.63-7.59 (m, 1H), 7.57- | |
| | | 7.52 (m, 1H), 7.42 (d, J = 8.8 Hz, | |
| | | 1H), 7.02-6.93 (m, 3H), 4.39-4.21 | |
| | | (m, 2H), 4.07-4.03 (m, 1H), 3.88- | |
| | | 3.68 (m, 2H), 3.69 (s, 2H), 2.97- | |
| | | 2.90 (m, 1H), 2.87-2.69 (m, 4H), | |
| | | 2.43 (s, 3H), 2.32-2.14 (m, 2H), | |
| | | 2.02-1.85 (m, 7H). | |
| 341 | 735.3 | ¹H NMR (500 MHz, DMSO-d₆) δ | 5 |
| | | 12.00 (s, 1H), 10.69 (s, 1H), 8.23 (d, | |
| | | J = 2.1 Hz, 1H), 7.92-7.89 (m, 1H), | |
| | | 7.78-7.73 (m, 1H), 7.70 (d, J = 2.1 | |
| | | Hz, 1H), 7.47-7.41 (m, 2H), 7.02- | |
| | | 6.90 (m, 5H), 4.39-4.21 (m, 2H), | |
| | | 4.07-4.03 (m, 1H), 3.88-3.68 (m, | |
| | | 2H), 3.69 (s, 2H), 3.23-2.89 (m, | |
| | | 3H), 2.73 (s, 3H), 2.69-2.61 (m, | |
| | | 2H), 2.46 (s, 3H), 2.32-2.14 (m, | |
| | | 2H), 2.02-1.85 (m, 7H). | |
| 342 | 721.3 | ¹H NMR (500 MHz, DMSO-d₆) δ | 5 |
| | | 12.67 (s, 1H), 10.56 (s, 1H), 8.23 (d, | |
| | | J = 2.1 Hz, 1H), 7.92-7.89 (m, 2H), | |
| | | 7.68-7.66 (m, 1H), 7.62-7.59 (m, | |
| | | 1H), 7.49-7.40 (m, 3H), 7.25-7.24 | |
| | | (m, 1H), 7.02-6.93 (m, 3H), 4.39- | |
| | | 4.21 (m, 2H), 4.07-4.03 (m, 1H), | |
| | | 3.88-3.68 (m, 2H), 3.69 (s, 2H), | |
| | | 2.97-2.90 (m, 1H), 2.87-2.79 (m, | |
| | | 5H), 2.69-2.61 (m, 2H), 2.32-2.14 | |
| | | (m, 2H), 2.02-1.85 (m, 7H). | |
| 343 | 762.3 | ¹H NMR (500 MHz, DMSO-d₆) δ | 5 |
| | | 10.04 (s, 1H), 9.93 (s, 1H), 8.23 (d, J = | |
| | | 2.2 Hz, 1H), 7.92-7.89 (m, 1H) | |
| | | 7.65 (d, J = 2.1 Hz, 1H), 7.62-7.59 | |
| | | (m, 1H), 7.55 (d, J = 2.0 Hz, 1H), | |
| | | 7.47-7.40 (m, 2H), 7.25-7.24 (m, | |
| | | 1H), 7.02-6.93 (m, 3H), 4.39- | |
| | | 4.08 (m, 3H), 3.90-3.67 (m, 2H), | |
| | | 3.69 (s, 2H), 2.97-2.90 (m, 1H), | |
| | | 2.87-2.69 (m, 4H), 2.38-2.33 (m, | |
| | | 1H), 2.22 (s, 3H), 2.17-2.08 (m, | |
| | | 2H), 2.08-2.01 (m, 1H), 2.07-1.93 | |
| | | (m, 7H), 1.72-1.61 (m, 2H). | |
| 344 | 755.2 | ¹H NMR (500 MHz, DMSO-d₆) δ | 5 |
| | | 12.40 (s, 1H), 10.75 (s, 1H), 8.23 (d, | |
| | | J = 2.1 Hz, 1H), 7.92-7.91 (m, 1H), | |
| | | 7.87-7.82 (m, 2H), 7.62-7.59 (m, | |
| | | 1H), 7.47-7.40 (m, 2H), 7.25-7.24 | |
| | | (m, 1H), 7.02-6.93 (m, 3H), 4.39- | |
| | | 4.21 (m, 2H), 4.07-4.03 (m, 1H), | |
| | | 3.88-3.68 (m, 2H), 3.69 (s, 2H), | |
| | | 2.97-2.90 (m, 1H), 2.87-2.69 (m, | |
| | | 4H), 2.44 (s, 3H), 2.32-2.14 (m, | |
| | | 2H), 2.00-1.82 (m, 7H). | |
| 345 | 737.3 | ¹H NMR (500 MHz, DMSO-d₆) δ | 5 |
| | | 10.74 (s, 1H), 10.63 (s, 1H), 9.97 (s, | |
| | | 1H), 8.23 (d, J = 2.1 Hz, 1H), 8.16 (d, | |
| | | J = 2.1 Hz, 1H), 7.92-7.89 (m, 1H), | |
| | | 7.62-7.59 (m, 1H), 7.47-7.41 (m, | |
| | | 2H), 7.42 (d, J = 1.3 Hz, 1H), 7.25- | |
| | | 7.24 (m, 1H), 7.02-6.93 (m, 3H), | |
| | | 4.39-4.08 (m, 3H), 3.90-3.67 (m, | |
| | | 2H), 3.69 (s, 2H), 2.97-2.90 (m, | |
| | | 1H), 2.87-2.69 (m, 4H), 2.38-2.33 | |
| | | (m, 1H), 2.28 (s, 3H), 2.09-1.85 (m, | |
| | | 8H). | |
| 346 | 726.3 | ¹H NMR (500 MHz, DMSO-d₆) δ | 5 |
| | | 12.55 (s, 1H), 10.52 (s, 1H), 8.43 (d, | |
| | | J = 1.2 Hz, 1H), 7.97 (d, J = 1.8 Hz, | |
| | | 1H), 7.92-7.91 (m, 1H), 7.79-7.73 | |
| | | (m, 2H), 7.72 (d, J = 5.2 Hz, 1H), | |
| | | 7.63-7.53 (m, 2H), 7.46 (d, J = 8.8 | |
| | | Hz, 1H), 7.02-6.93 (m, 3H), 4.39- | |
| | | 4.21 (m, 2H), 4.05-3.98 (m, 1H), | |
| | | 3.88-3.68 (m, 2H), 3.69 (s, 2H), | |
| | | 2.97-2.90 (m, 1H), 2.87-2.79 (m, | |
| | | 3H), 2.82-2.77 (m, 1H), 2.69-2.61 | |
| | | (m, 2H), 2.32-2.14 (m, 2H), 2.02- | |
| | | 1.85 (m, 7H), 1.36-1.30 (m, 3H). | |
| 347 | 740.3 | ¹H NMR (500 MHz, DMSO-d₆) δ | 5 |
| | | 12.49 (s, 1H), 10.32 (s, 1H), 8.53 (d, | |
| | | J = 1.8 Hz, 1H), 7.97 (s, 1H), 7.92- | |
| | | 7.91 (m, 1H), 7.80-7.72 (m, 3H), | |
| | | 7.63-7.53 (m, 2H), 7.46 (d, J = 8.8 | |
| | | Hz, 1H), 7.02-6.96 (m, 1H), 6.99- | |
| | | 6.90 (m, 2H), 4.39-4.21 (m, 2H), | |
| | | 4.05-3.98 (m, 1H), 3.88-3.68 (m, | |
| | | 2H), 3.69 (s, 2H), 3.17-3.07 (m, | |
| | | 1H), 2.97-2.89 (m, 1H), 2.87-2.69 | |
| | | (m, 4H), 2.32-2.14 (m, 2H), 2.02- | |
| | | 1.85 (m, 7H), 1.69 (d, J = 6.5 Hz, | |
| | | 3H), 1.63 (d, J = 6.5 Hz, 3H). | |
| 348 | 738.3 | ¹H NMR (500 MHz, DMSO-d₆) δ | 5 |
| | | 12.61 (s, 1H), 10.32 (s, 1H), 8.49 (d, | |
| | | J = 1.8 Hz, 1H), 7.97 (d, J = 1.8 Hz, | |
| | | 1H), 7.92-7.91 (m, 1H), 7.80-7.73 | |
| | | (m, 2H), 7.76-7.73 (m, 1H), 7.63- | |
| | | 7.53 (m, 2H), 7.46 (d, J = 8.8 Hz, | |
| | | 1H), 7.02-6.93 (m, 3H), 4.39-4.21 | |
| | | (m, 2H), 4.05-3.98 (m, 1H), 3.88- | |
| | | 3.68 (m, 2H), 3.69 (s, 2H), 2.97- | |
| | | 2.90 (m, 1H), 2.87-2.69 (m, 4H), | |
| | | 2.35-2.23 (m, 3H), 2.02-1.85 (m, | |
| | | 7H), 1.13-0.89 (m, 4H). | |
| 349 | 778.3 | ¹H NMR (500 MHz, DMSO-d₆) δ | 3 |
| | | 12.97 (s, 1H), 11.15 (s, 1H), 8.23 (d, | |
| | | J = 2.1 Hz, 1H), 8.06 (d, J = 2.1 Hz, | |
| | | 1H), 7.92-7.89 (m, 1H), 7.53-7.46 | |
| | | (m, 2H), 7.45-7.40 (m, 2H), 7.39- | |
| | | 7.30 (m, 2H), 7.21-6.93 (m, 1H), | |
| | | 6.91-6.47 (m, 3H), 5.38-5.27 (m, | |
| | | 2H), 4.39-4.08 (m, 3H), 3.87-3.74 | |
| | | (m, 4H), 3.34-3.27 (m, 2H), 3.04- | |
| | | 2.91 (m, 4H), 2.73 (s, 3H), 2.41 (s, | |
| | | 3H), 2.19-2.10 (m, 2H). | |
| 350 | 753.3 | ¹H NMR (500 MHz, DMSO-d₆) δ | 3 |
| | | 12.44 (s, 1H), 10.82 (s, 1H), 8.23 (d, | |
| | | J = 2.1 Hz, 1H), 7.92-7.89 (m, 1H), | |
| | | 7.82 (d, J = 2.2 Hz, 1H), 7.67 (d, J = | |
| | | 8.0 Hz, 1H), 7.57-7.55 (s, 1H), 7.53- | |
| | | 7.51 (m, 1H), 7.45-7.40 (m, 2H), | |
| | | 7.39-7.33 (m, 2H), 6.88-6.47 (m, | |
| | | 2H), 5.42-5.33 (m, 2H), 5.11-4.79 | |
| | | (m, 1H), 4.30-4.00 (m, 2H), 3.87- | |
| | | 3.74 (m, 4H), 3.34-3.27 (m, 2H), | |
| | | 3.04-2.91 (m, 4H), 2.46 (s, 3H), | |
| | | 2.43 (s, 3H), 2.19-2.10 (m, 2H). | |
| 351 | 764.2 | ¹H NMR (500 MHz, DMSO-d₆) δ | 3 |
| | | 10.17 (s, 1H), 9.97 (s, 1H), 8.22- | |
| | | 8.23 (m, 1H), 7.92-7.89 (m, 1H), | |
| | | 7.70 (s, 1H), 7.63-7.56 (m, 2H), | |
| | | 7.53-7.46 (m, 2H), 7.45-7.40 (m, | |
| | | 2H), 7.35 (d, J = 9.6 Hz, 1H), 6.88- | |
| | | 6.47 (m, 2H), 5.38-5.27 (m, 2H), | |
| | | 4.39-4.08 (m, 3H), 3.90-3.67 (m, | |
| | | 4H), 3.34-3.27 (m, 2H), 3.04-2.91 | |
| | | (m, 4H), 2.45 (s, 3H), 2.38-2.33 (m, | |
| | | 1H), 2.19-1.69 (m, 5H). | |
| 352 | 798.2 | ¹H NMR (500 MHz, DMSO-d₆) δ | 3 |
| | | 12.95 (s, 1H), 10.87 (s, 1H), 8.23 (d, | |
| | | J = 2.1 Hz, 1H), 7.92-7.91 (m, 1H), | |
| | | 7.86 (d, J = 2.1 Hz, 1H), 7.62 (d, J = | |
| | | 2.0 Hz, 1H), 7.57-7.56 (m, 1H), | |
| | | 7.53-7.51 (m, 1H), 7.46-7.40 (m, | |
| | | 2H), 7.35 (d, J = 2.0 Hz, 2H), 7.00 (s, | |
| | | 1H), 6.88-6.47 (m, 2H), 5.33 (d, J = | |
| | | 6.0 Hz, 2H), 4.39-4.08 (m, 3H), 3.87- | |
| | | 3.74 (m, 4H), 3.34-3.27 (m, 2H), | |
| | | 3.04-2.91 (m, 4H), 2.46 (s, 3H), | |
| | | 2.19-2.10 (m, 2H). | |
| 353 | 737.3 | ¹H NMR (500 MHz, DMSO-d₆) δ | 3 |
| | | 12.44 (s, 1H), 10.82 (s, 1H), 8.22- | |
| | | 8.23 (m, 1H), 7.92-7.89 (m, 1H), | |
| | | 7.82 (d, J = 2.2 Hz, 1H), 7.58-7.46 | |
| | | (m, 3H), 7.45-7.40 (m, 2H), 7.35 (d, | |
| | | J = 9.6 Hz, 1H), 6.91-6.47 (m, 3H), | |
| | | 5.38-5.27 (m, 2H), 4.39-4.08 (m, | |
| | | 3H), 3.87-3.74 (m, 4H), 3.34-3.27 | |
| | | (m, 2H), 3.04-2.91 (m, 4H), 2.43 (s, | |
| | | 3H), 2.41 (s, 3H), 2.19-2.10 (m, 2H). | |
| 354 | 753.3 | ¹H NMR (500 MHz, DMSO-d₆) δ | 3 |
| | | 12.00 (s, 1H), 10.69 (s, 1H), 8.23 (d, | |
| | | J = 2.1 Hz, 1H), 7.92-7.89 (m, 1H), | |
| | | 7.70 (d, J = 2.1 Hz, 1H), 7.53 (d, J = | |
| | | 9.7 Hz, 1H), 7.53-7.46 (m, 2H), | |
| | | 7.45-7.40 (m, 2H), 7.36 (d, J = 9.6 | |
| | | Hz, 1H), 6.88-6.85 (m, 1H), 6.79 (s, | |
| | | 1H), 6.47-6.45 (m, 1H), 5.33 (d, J = | |
| | | 6.0 Hz, 2H), 4.39-4.08 (m, 3H), 3.87- | |
| | | 3.74 (m, 4H), 3.34-3.27 (m, 2H), | |
| | | 3.04-2.91 (m, 4H), 2.73 (s, 3H), | |
| | | 2.46 (s, 3H), 2.19-2.10 (m, 2H). | |
| 355 | 719.3 | ¹H NMR (500 MHz, DMSO-d₆) δ | 3 |
| | | 12.67 (s, 1H), 10.56 (s, 1H), 8.23 (d, | |
| | | J = 2.1 Hz, 1H), 7.92-7.89 (m, 2H), | |
| | | 7.68-7.66 (m, 1H), 7.55-7.44 (m, | |
| | | 3H), 7.44-7.40 (m, 2H), 7.35 (d, J = | |
| | | 9.6 Hz, 1H), 6.91-6.47 (m, 3H), 5.38- | |
| | | 5.27 (m, 2H), 4.39-4.08 (m, 3H), | |
| | | 3.87-3.74 (m, 4H), 3.34-3.27 (m, | |
| | | 2H), 3.04-2.91 (m, 4H), 2.84 (s, | |
| | | 3H), 2.44 (s, 3H), 2.13-2.08 (m, | |
| | | 2H). | |
| 356 | 773.3 | ¹H NMR (500 MHz, DMSO-d₆) δ | 3 |
| | | 13.03 (s, 1H), 10.94 (s, 1H), 8.23 (d, | |
| | | J = 2.1 Hz, 1H), 8.12 (d, J = 2.1 Hz, | |
| | | 1H), 7.92-7.89 (m, 1H), 7.67-7.65 | |
| | | (m, 1H), 7.60 (d, J = 7.9 Hz, 1H), | |
| | | 7.55-7.49 (m, 2H), 7.45-7.40 (m, | |
| | | 2H), 7.35 (d, J = 9.6 Hz, 1H), 6.91- | |
| | | 6.47 (m, 3H), 5.38-5.27 (m, 2H), | |
| | | 4.39-4.08 (m, 3H), 3.87-3.74 (m, | |
| | | 4H), 3.34-3.27 (m, 2H), 3.04-2.91 | |
| | | (m, 4H), 2.43 (s, 3H), 2.16-2.10 (m, | |
| | | 2H). | |
| 357 | 753.3 | ¹H NMR (500 MHz, DMSO-d₆) δ | 3 |
| | | 12.05 (s, 1H), 10.46 (s, 1H), 8.32 (d, | |
| | | J = 2.1 Hz, 1H), 7.95 (d, J = 1.8 Hz, | |
| | | 1H), 7.83 (d, J = 2.1 Hz, 1H), 7.59- | |
| | | 7.40 (m, 5H), 7.36 (d, J = 9.6 Hz, | |
| | | 1H), 6.88-6.85 (m, 1H), 6.79 (s, | |
| | | 1H), 6.47-6.45 (m, 1H), 5.33 (d, J = | |
| | | 6.0 Hz, 2H), 5.11-4.79 (m, 1H), | |
| | | 4.34-3.98 (m, 2H), 3.87-3.74 (m, | |
| | | 4H), 3.34-3.27 (m, 2H), 3.04-2.91 | |
| | | (m, 4H), 2.72 (s, 3H), 2.45 (s, 3H), | |
| | | 2.19-2.11 (m, 2H). | |
| 358 | 719.3 | ¹H NMR (500 MHz, DMSO-d₆) δ | 3 |
| | | 8.23 (d, J = 2.1 Hz, 1H), 7.92-7.89 | |
| | | (m, 2H), 7.68-7.66 (m, 1H), 7.55- | |
| | | 7.44 (m, 3H), 7.44-7.40 (m, 2H), | |
| | | 7.35 (d, J = 9.6 Hz, 1H), 6.91-6.47 | |
| | | (m, 3H), 5.38-5.27 (m, 2H), 4.39- | |
| | | 4.08 (m, 3H), 3.87-3.74 (m, 4H), | |
| | | 3.34-3.27 (m, 2H), 3.04-2.91 (m, | |
| | | 4H), 2.84 (s, 3H), 2.41 (s, 3H), 2.19- | |
| | | 2.10 (m, 2H). | |
| 359 | 773.3 | ¹H NMR (500 MHz, DMSO-d₆) δ | 3 |
| | | 8.23 (d, J = 2.1 Hz, 1H), 8.12 (d, J = | |
| | | 2.1 Hz, 1H), 7.92-7.89 (m, 1H), | |
| | | 7.67-7.65 (m, 1H), 7.60 (d, J = 7.9 | |
| | | Hz, 1H), 7.55-7.49 (m, 2H), 7.45- | |
| | | 7.40 (m, 2H), 7.35 (d, J = 9.6 Hz, | |
| | | 1H), 6.91-6.47 (m, 3H), 5.38-5.27 | |
| | | (m, 2H), 4.39-4.08 (m, 3H), 3.87- | |
| | | 3.74 (m, 4H), 3.34-3.27 (m, 2H), | |
| | | 3.04-2.91 (m, 4H), 2.41 (s, 3H), | |
| | | 2.19-2.10 (m, 2H). | |
| 360 | 737.3 | ¹H NMR (500 MHz, DMSO-d₆) δ | 3 |
| | | 8.22-8.23 (m, 1H), 7.92-7.89 (m, | |
| | | 1H), 7.82 (d, J = 2.2 Hz, 1H), 7.58- | |
| | | 7.46 (m, 3H), 7.45-7.40 (m, 2H), | |
| | | 7.35 (d, J = 9.6 Hz, 1H), 6.91-6.47 | |
| | | (m, 3H), 5.38-5.27 (m, 2H), 4.39- | |
| | | 4.08 (m, 3H), 3.87-3.74 (m, 4H), | |
| | | 3.34-3.27 (m, 2H), 3.04-2.91 (m, | |
| | | 4H), 2.43 (s, 3H), 2.41 (s, 3H), 2.19- | |
| | | 2.10 (m, 2H). | |
| 361 | 703.3 | ¹H NMR (500 MHz, DMSO-d₆) δ | 3 |
| | | 8.42 (d, J = 1.8 Hz, 1H), 7.97 (s, 1H), | |
| | | 7.91-7.90 (m, 1H), 7.70 (d, J = 7.9 | |
| | | Hz, 1H), 7.55-7.53 (m, 1H), 7.53- | |
| | | 7.40 (m, 3H), 6.86-6.84 (m, 1H), | |
| | | 6.47-6.45 (m, 1H), 5.31 (d, J = 1.3 | |
| | | Hz, 2H), 5.11-4.79 (m, 1H), 4.34- | |
| | | 3.98 (m, 2H), 3.90-3.67 (m, 4H), | |
| | | 3.34-3.27 (m, 2H), 3.04-2.91 (m, | |
| | | 4H), 2.43 (s, 3H), 2.38-2.33 (m, | |
| | | 1H), 2.29 (s, 3H), 2.09-2.02 (m, | |
| | | 1H). | |
| 362 | 707.2 | ¹H NMR (500 MHz, DMSO-d₆) δ | 3 |
| | | 8.24 (d, J = 2.2 Hz, 1H), 7.97 (d, J = | |
| | | 1.8 Hz, 1H), 7.80-7.77 (m, 1H), | |
| | | 7.59-7.57 (m, 1H), 7.53-7.40 (m, | |
| | | 3H), 6.90-6.81 (m, 2H), 6.47-6.45 | |
| | | (m, 1H), 5.33 (s, 2H), 5.11-4.79 (m, | |
| | | 1H), 4.34-3.98 (m, 2H), 3.87-3.74 | |
| | | (m, 4H), 3.34-3.27 (m, 2H), 3.04- | |
| | | 2.91 (m, 4H), 2.43 (s, 3H), 2.19- | |
| | | 2.10 (m, 2H). | |
| 363 | 707.2 | ¹H NMR (500 MHz, DMSO-d₆) δ | 3 |
| | | 8.29 (d, J = 2.1 Hz, 1H), 7.92-7.89 | |
| | | (m, 1H), 7.82 (d, J = 2.2 Hz, 1H), | |
| | | 7.57-7.55 (s, 1H), 7.53-7.51 (m, | |
| | | 1H), 7.45-7.40 (m, 2H), 6.90-6.81 | |
| | | (m, 2H), 6.47-6.45 (m, 1H), 5.33 (s, | |
| | | 2H), 4.39-4.08 (m, 3H), 3.87-3.74 | |
| | | (m, 4H), 3.34-3.27 (m, 2H), 3.04- | |
| | | 2.91 (m, 4H), 2.48 (s, 3H), 2.19- | |
| | | 2.10 (m, 2H). | |
| 364 | 703.3 | ¹H NMR (500 MHz, DMSO-d₆) δ | 3 |
| | | 12.00 (s, 1H), 10.69 (s, 1H), 8.23 (d, | |
| | | J = 2.1 Hz, 1H), 7.92-7.89 (m, 1H), | |
| | | 7.70 (d, J = 2.1 Hz, 1H), 7.53-7.46 | |
| | | (m, 2H), 7.45-7.40 (m, 2H), 6.90- | |
| | | 6.81 (m, 2H), 6.47-6.45 (m, 1H), | |
| | | 5.33 (s, 2H), 4.39-4.08 (m, 3H), 3.87- | |
| | | 3.74 (m, 4H), 3.34-3.27 (m, 2H), | |
| | | 3.04-2.91 (m, 4H), 2.78 (s, 3H), | |
| | | 2.49 (s, 3H), 2.17-2.07 (m, 2H). | |
| 365 | 757.2 | ¹H NMR (500 MHz, DMSO-d₆) δ | 3 |
| | | 8.25 (d, J = 2.0 Hz, 1H), 7.92-7.89 | |
| | | (m, 2H), 7.52-7.49 (m, 2H), 7.45- | |
| | | 7.40 (m, 2H), 6.90-6.81 (m, 2H), | |
| | | 6.47-6.45 (m, 1H), 5.33 (s, 2H), | |
| | | 4.39-4.08 (m, 3H), 3.87-3.74 (m, | |
| | | 4H), 3.34-3.27 (m, 2H), 3.04-2.92 | |
| | | (m, 4H), 2.90 (s, 3H), 2.19-2.10 (m, | |
| | | 2H). | |
| 366 | 715.3 | ¹H NMR (500 MHz, DMSO-d₆) δ | 3 |
| | | 8.20 (d, J = 2.1 Hz, 1H), 8.18-8.07 | |
| | | (m, 2H), 7.92-7.89 (m, 2H), 7.74 (d, | |
| | | J = 8.3 Hz, 1H), 7.68-7.66 (m, 1H), | |
| | | 7.59-7.55 (m, 1H), 7.55-7.40 (m, | |
| | | 5H), 7.35 (d, J = 8.2 Hz, 1H), 6.88- | |
| | | 6.47 (m, 2H), 5.56-5.45 (m, 2H), | |
| | | 5.11-4.79 (m, 1H), 4.32-4.28 (m, | |
| | | 1H), 4.08-4.04 (m, 1H), 4.01-3.89 | |
| | | (m, 2H), 3.87-3.73 (m, 2H), 3.34- | |
| | | 3.27 (m, 2H), 3.04-2.92 (m, 4H), | |
| | | 2.84 (s, 3H), 2.19-2.10 (m, 2H). | |
| 367 | 737.3 | ¹H NMR (500 MHz, DMSO-d₆) δ | 3 |
| | | 12.44 (s, 1H), 10.82 (s, 1H), 8.23 (d, | |
| | | J = 2.0 Hz, 1H), 8.11 (s, 1H), 7.92- | |
| | | 7.89 (m, 1H), 7.82 (d, J = 2.2 Hz, | |
| | | 1H), 7.63 (d, J = 8.0 Hz, 1H), 7.57- | |
| | | 7.55 (s, 1H), 7.53-7.51 (m, 1H), | |
| | | 7.45-7.40 (m, 2H), 7.37 (d, J = 8.6 | |
| | | Hz, 1H), 6.88-6.47 (m, 2H), 5.55- | |
| | | 5.43 (m, 2H), 5.11-4.79 (m, 1H), | |
| | | 4.32-4.28 (m, 1H), 4.07-4.03 (m, | |
| | | 1H), 3.97 (d, J = 13.8 Hz, 1H), 3.92 | |
| | | (d, J = 13.8 Hz, 1H), 3.90 (s, 3H), | |
| | | 3.87-3.73 (m, 2H), 3.34-3.27 (m, | |
| | | 2H), 3.04-2.91 (m, 4H), 2.43 (s, | |
| | | 3H), 2.19-2.10 (m, 2H). | |
| 368 | 709.3 | ¹H NMR (500 MHz, DMSO-d₆) δ | 3 |
| | | 8.30 (d, J = 8.0 Hz, 1H), 8.23 (d, J = | |
| | | 2.0 Hz, 1H), 8.13-8.08 (m, 1H), | |
| | | 8.00-7.95 (m, 1H), 7.92-7.89 (m, | |
| | | 2H), 7.72-7.62 (m, 2H), 7.53-7.40 | |
| | | (m, 5H), 6.88-6.47 (m, 2H), 5.62 (d, | |
| | | J = 1.3 Hz, 2H), 5.11-4.79 (m, 1H), | |
| | | 4.32-4.28 (m, 1H), 4.07-4.03 (m, | |
| | | 1H), 3.97 (d, J = 13.7 Hz, 1H), 3.95- | |
| | | 3.82 (m, 2H), 3.75-3.69 (m, 1H), | |
| | | 3.34-3.27 (m, 2H), 3.04-2.91 (m, | |
| | | 4H), 2.80 (s, 3H), 2.36-2.03 (m, | |
| | | 2H). | |
| 369 | 711.3 | ¹H NMR (500 MHz, DMSO-d₆) δ | 3 |
| | | 12.67 (s, 1H), 10.56 (s, 1H), 8.23 (d, | |
| | | J = 2.1 Hz, 1H), 7.92-7.91 (m, 2H), | |
| | | 7.87-7.82 (m, 1H), 7.68-7.66 (m, | |
| | | 1H), 7.53-7.34 (m, 5H), 7.30-7.25 | |
| | | (m, 1H), 6.88-6.47 (m, 2H), 5.38 (d, | |
| | | J = 1.3 Hz, 2H), 4.39-4.08 (m, 3H), | |
| | | 3.87-3.74 (m, 4H), 3.34-3.27 (m, | |
| | | 2H), 3.04-2.97 (m, 2H), 2.91-2.79 | |
| | | (m, 8H), 2.19-2.10 (m, 2H). | |
| 370 | 721.3 | ¹H NMR (500 MHz, DMSO-d₆) δ | 2 |
| | | 12.60 (s, 1H), 10.50 (s, 1H), 8.42 (d, | |
| | | J = 1.8 Hz, 1H), 7.97 (s, 1H), 7.91- | |
| | | 7.90 (m, 1H), 7.70 (d, J = 8.0 Hz, | |
| | | 1H), 7.54-7.43 (m, 4H), 7.35 (d, J = | |
| | | 9.6 Hz, 1H), 7.19-7.18 (m, 1H), | |
| | | 6.89 (s, 1H), 6.81-6.78 (m, 1H), | |
| | | 5.33 (s, 1H), 5.11-4.79 (m, 1H), | |
| | | 4.34-3.98 (m, 2H), 3.87-3.68 (m, | |
| | | 4H), 3.11-3.00 (m, 3H), 2.87-2.78 | |
| | | (m, 2H), 2.43 (s, 3H), 2.41 (s, 3H), | |
| | | 2.19-2.10 (m, 2H), 2.08-1.90 (m, | |
| | | 5H). | |
| 371 | 775.3 | ¹H NMR (500 MHz, DMSO-d₆) δ | 2 |
| | | 13.14 (s, 1H), 10.59 (s, 1H), 8.50 (d, | |
| | | J = 1.3 Hz, 1H), 7.97 (d, J = 1.8 Hz, | |
| | | 1H), 7.95-7.90 (m, 1H), 7.79 (d, J = | |
| | | 7.9 Hz, 1H), 7.54-7.43 (m, 4H), | |
| | | 7.35 (d, J = 9.6 Hz, 1H), 7.19-7.18 | |
| | | (m, 1H), 6.89 (s, 1H), 6.81-6.78 (m, | |
| | | 1H), 5.33 (s, 2H), 5.11-4.79 (m, | |
| | | 1H), 4.34-3.98 (m, 2H), 3.87-3.68 | |
| | | (m, 4H), 3.11-3.00 (m, 3H), 2.87- | |
| | | 2.78 (m, 2H), 2.41 (s, 3H), 2.19- | |
| | | 2.10 (m, 2H), 2.03-1.86 (m, 4H). | |
| 372 | 737.3 | ¹H NMR (500 MHz, DMSO-d₆) δ | 2 |
| | | 12.40 (s, 1H), 10.64 (s, 1H), 8.24 (d, | |
| | | J = 2.2 Hz, 1H), 7.97 (d, J = 1.8 Hz, | |
| | | 1H), 7.80-7.77 (m, 1H), 7.60-7.43 | |
| | | (m, 4H), 7.35 (d, J = 9.6 Hz, 1H), | |
| | | 7.19-7.18 (m, 1H), 6.89 (s, 1H), | |
| | | 6.81-6.78 (m, 1H), 5.53 (s, 2H), | |
| | | 5.11-4.79 (m, 1H), 4.34-3.98 (m, | |
| | | 2H), 3.87-3.68 (m, 4H), 3.11-3.00 | |
| | | (m, 3H), 2.87-2.78 (m, 2H), 2.42 (s, | |
| | | 3H), 2.41 (s, 3H), 2.19-2.10 (m, | |
| | | 2H), 2.03-1.86 (m, 4H). | |
| 373 | 705.3 | ¹H NMR (500 MHz, DMSO-d₆) δ | 2 |
| | | 12.60 (s, 1H), 10.50 (s, 1H), 8.42 (d, | |
| | | J = 1.8 Hz, 1H), 7.97 (s, 1H), 7.91- | |
| | | 7.90 (m, 1H), 7.70 (d, J = 8.0 Hz, | |
| | | 1H), 7.55-7.53 (m, 1H), 7.53-7.47 | |
| | | (m, 2H), 7.19-7.18 (m, 1H), 6.81- | |
| | | 6.79 (m, 1H), 5.31 (s, 2H), 5.11- | |
| | | 4.79 (m, 1H), 4.34-3.98 (m, 2H), | |
| | | 3.90-3.67 (m, 2H), 3.69 (s, 2H), | |
| | | 3.11-3.00 (m, 3H), 2.87-2.78 (m, | |
| | | 2H), 2.43 (s, 3H), 2.36-2.27 (m, | |
| | | 4H), 2.09-1.90 (m, 5H). | |
| 374 | 709.2 | ¹H NMR (500 MHz, DMSO-d₆) δ | 2 |
| | | 12.40 (s, 1H), 10.64 (s, 1H), 8.24 (d, | |
| | | J = 2.2 Hz, 1H), 7.97 (d, J = 1.8 Hz, | |
| | | 1H), 7.80-7.77 (m, 1H), 7.59-7.57 | |
| | | (m, 1H), 7.53-7.47 (m, 2H), 7.19- | |
| | | 7.18 (m, 1H), 6.87 (d, J = 8.0 Hz, | |
| | | 1H), 6.81-6.79 (m, 1H), 5.33 (s, | |
| | | 2H), 5.11-4.79 (m, 1H), 4.34-3.98 | |
| | | (m, 2H), 3.87-3.68 (m, 4H), 3.11- | |
| | | 3.00 (m, 3H), 2.87-2.78 (m, 2H), | |
| | | 2.42 (s, 3H), 2.19-2.10 (m, 2H), | |
| | | 2.03-1.86 (m, 4H). | |
| 375 | 709.2 | ¹H NMR (500 MHz, DMSO-d₆) δ | 2 |
| | | 12.40 (s, 1H), 10.64 (s, 1H), 8.24 (d, | |
| | | J = 2.2 Hz, 1H), 7.97 (d, J = 1.8 Hz, | |
| | | 1H), 7.80-7.77 (m, 1H), 7.59-7.57 | |
| | | (m, 1H), 7.53-7.47 (m, 2H), 7.19- | |
| | | 7.18 (m, 1H), 6.87 (d, J = 8.0 Hz, | |
| | | 1H), 6.81-6.79 (m, 1H), 5.33 (s, | |
| | | 2H), 5.11-4.79 (m, 1H), 4.34-3.98 | |
| | | (m, 2H), 3.87-3.68 (m, 4H), 3.11- | |
| | | 3.00 (m, 3H), 2.87-2.78 (m, 2H), | |
| | | 2.42 (s, 3H), 2.19-2.10 (m, 2H), | |
| | | 2.03-1.86 (m, 4H). | |
| 376 | 705.3 | ¹H NMR (500 MHz, DMSO-d₆) δ | 2 |
| | | 12.00 (s, 1H), 10.69 (s, 1H), 8.23 (d, | |
| | | J = 2.1 Hz, 1H), 7.92-7.89 (m, 1H), | |
| | | 7.70 (d, J = 2.1 Hz, 1H), 7.53-7.46 | |
| | | (m, 2H), 7.42 (d, J = 8.8 Hz, 1H), | |
| | | 7.19-7.18 (m, 1H), 6.87 (d, J = 8.0 | |
| | | Hz, 1H), 6.81-6.79 (m, 1H), 5.33 (s, | |
| | | 2H), 4.39-4.08 (m, 3H), 3.87-3.68 | |
| | | (m, 4H), 3.11-3.00 (m, 3H), 2.87- | |
| | | 2.78 (m, 2H), 2.73 (s, 3H), 2.46 (s, | |
| | | 3H), 2.19-2.10 (m, 2H), 2.03-1.86 | |
| | | (m, 4H). | |
| 377 | 759.2 | ¹H NMR (500 MHz, DMSO-d₆) δ | 2 |
| | | 8.23 (d, J = 2.1 Hz, 1H), 7.92-7.89 | |
| | | (m, 2H), 7.53-7.46 (m, 2H), 7.42 (d, | |
| | | J = 8.8 Hz, 1H), 7.19-7.18 (m, 1H), | |
| | | 6.87 (d, J = 8.0 Hz, 1H), 6.81-6.79 | |
| | | (m, 1H), 5.33 (s, 2H), 4.39-4.08 (m, | |
| | | 3H), 3.87-3.68 (m, 4H), 3.10-3.01 | |
| | | (m, 3H), 2.87-2.78 (m, 2H), 2.73 (s, | |
| | | 3H), 2.19-2.10 (m, 2H), 2.03-1.86 | |
| | | (m, 4H). | |
| 378 | 717.3 | ¹H NMR (500 MHz, DMSO-d₆) δ | 2 |
| | | 8.23 (d, J = 2.1 Hz, 1H), 8.18-8.13 | |
| | | (m, 1H), 8.13-8.07 (m, 1H), 7.92- | |
| | | 7.89 (m, 2H), 7.74 (d, J = 8.3 Hz, | |
| | | 1H), 7.68-7.66 (m, 1H), 7.62-7.55 | |
| | | (s, 1H), 7.55-7.45 (m, 3H), 7.42 (d, | |
| | | J = 8.8 Hz, 1H), 7.35 (d, J = 8.2 Hz, | |
| | | 1H), 7.19-7.18 (m, 1H), 6.83-6.80 | |
| | | (m, 1H), 5.51 (s, 2H), 4.39-4.08 (m, | |
| | | 3H), 3.87-3.68 (m, 4H), 3.11-3.01 | |
| | | (m, 5H), 2.87-2.78 (m, 2H), 2.19- | |
| | | 2.10 (m, 2H), 2.08-1.90 (m, 5H). | |
| 379 | 739.2 | ¹H NMR (500 MHz, DMSO-d₆) δ | 2 |
| | | 8.23 (d, J = 2.0 Hz, 1H), 8.11 (s, 1H), | |
| | | 7.92-7.89 (m, 1H), 7.82 (d, J = 2.2 | |
| | | Hz, 1H), 7.63 (d, J = 8.0 Hz, 1H), | |
| | | 7.57-7.55 (s, 1H), 7.53-7.51 (m, | |
| | | 1H), 7.42-7.37 (m, 2H), 7.19-7.18 | |
| | | (m, 1H), 6.80-6.77 (m, 1H), 5.49 (s, | |
| | | 2H), 4.39-4.08 (m, 3H), 3.90 (s, | |
| | | 3H), 3.87-3.68 (m, 4H), 3.11-3.03 | |
| | | (m, 3H), 2.87-2.78 (m, 2H), 2.43 (s, | |
| | | 3H), 2.19-2.10 (m, 2H), 2.03-1.86 | |
| | | (m, 4H). | |
| 380 | 711.3 | ¹H NMR (500 MHz, Chloroform-d) δ | 2 |
| | | 12.01 (s, 1H), 10.39 (s, 1H), 8.24 (d, | |
| | | J = 8.1 Hz, 1H), 8.07-8.05 (m, 2H), | |
| | | 7.93 (dd, J = 8.1, 1.2 Hz, 1H), 7.86- | |
| | | 7.83 (m, 2H), 7.70-7.62 (m, 2H), | |
| | | 7.50-7.46 (m, 2H), 7.44-7.37 (m, | |
| | | 2H), 7.13-7.11 (m, 2H), 5.62 (s, | |
| | | 2H), 4.38-4.17 (m, 3H), 3.80-3.69 | |
| | | (m, 4H), 3.03-2.78 (m, 6H), 2.65- | |
| | | 2.34 (m, 3H), 2.02-1.93 (m, 6H). | |
| 381 | 713.3 | ¹H NMR (500 MHz, Chloroform-d) δ | 2 |
| | | 11.91 (s, 1H), 10.24 (s, 1H), 8.00- | |
| | | 7.88 (m, 2H), 7.83-7.77 (m, 2H), | |
| | | 7.63 (d, J = 7.8 Hz, 1H), 7.47-7.32 | |
| | | (m, 4H), 7.21 (dd, J = 8.1, 1.2 Hz, | |
| | | 1H), 7.18-7.13 (m, 1H), 6.81 (d, J = | |
| | | 7.7 Hz, 1H), 5.46 (s, 2H), 4.35-4.17 | |
| | | (m, 3H), 3.81-3.75 (s, 4H), 3.03-2.90 | |
| | | (m, 3H), 2.80-2.78 (m, 5H), 2.65- | |
| | | 2.37 (m, 4H), 2.05-1.94 (m, 4H). | |
| 382 | 716.3 | ¹H NMR (500 MHz, Chloroform-d) δ | 5 |
| | | 8.39 (d, J = 2.0 Hz, 1H), 8.15 (d, J = | |
| | | 1.9 Hz, 1H), 7.85-7.84 (m, 1H), 7.65- | |
| | | 7.61 (m, 2H), 7.50-7.48 (m, 2H), | |
| | | 7.42 (d, J = 8.5 Hz, 1H), 7.03-6.98 | |
| | | (m, 3H), 6.17-6.13 (m, 1H), 4.35- | |
| | | 4.31 (m, 1H), 4.28-4.21 (m, 2H), | |
| | | 3.90-3.82 (m, 4H), 3.34-3.31 (m, | |
| | | 2H), 2.94-2.85 (m, 5H), 2.77-2.73 | |
| | | (m, 2H), 2.65 (s, 2H), 2.32-2.20 (m, | |
| | | 2H). | |
| 383 | 716.3 | ¹H NMR (500 MHz, Chloroform-d) δ | 5 |
| | | 8.39 (d, J = 2.0 Hz, 1H), 8.15 (d, J = | |
| | | 1.9 Hz, 1H), 7.85-7.84 (m, 1H), 7.65- | |
| | | 7.61 (m, 2H), 7.50-7.48 (m, 2H), | |
| | | 7.42 (d, J = 8.5 Hz, 1H), 7.03-6.98 | |
| | | (m, 3H), 6.17-6.13 (m, 1H), 4.35- | |
| | | 4.31 (m, 1H), 4.28-4.21 (m, 2H), | |
| | | 3.90-3.82 (m, 4H), 3.34-3.31 (m, | |
| | | 2H), 2.94-2.85 (m, 5H), 2.77-2.73 | |
| | | (m, 2H), 2.65 (s, 2H), 2.32-2.20 (m, | |
| | | 2H). | |
| 384 | 733.3 | ¹H NMR (500 MHz, DMSO-d6) δ | 5 |
| | | 12.38 (s, 1H), 10.76 (s, 1H), 8.32 (d, | |
| | | J = 2.1 Hz, 1H), 7.95 (d, J = 1.8 Hz, | |
| | | 1H), 7.83 (d, J = 2.1 Hz, 1H), 7.62- | |
| | | 7.56 (m, 2H), 7.49-7.45 (m, 2H), | |
| | | 7.25-7.24 (m, 1H), 7.20-7.16 (m, | |
| | | 1H), 7.05-6.98 (m, 2H), 6.11-6.08 | |
| | | (m, 1H), 4.39-3.68 (m, 7H), 3.35- | |
| | | 2.72 (m, 6H), 2.70 (s, 3H), 2.44 (s, | |
| | | 3H), 2.32-2.14 (m, 2H), 1.99 (s, | |
| | | 3H). | |
| 385 | 733.3 | ¹H NMR (500 MHz, DMSO-d6) δ | 5 |
| | | 12.38 (s, 1H), 10.76 (s, 1H), 8.32 (d, | |
| | | J = 2.1 Hz, 1H), 7.95 (d, J = 1.8 Hz, | |
| | | 1H), 7.83 (d, J = 2.1 Hz, 1H), 7.62- | |
| | | 7.56 (m, 2H), 7.49-7.45 (m, 2H), | |
| | | 7.25-7.24 (m, 1H), 7.20-7.16 (m, | |
| | | 1H), 7.05-6.98 (m, 2H), 6.11-6.08 | |
| | | (m, 1H), 4.39-3.68 (m, 7H), 3.35- | |
| | | 2.72 (m, 6H), 2.70 (s, 3H), 2.44 (s, | |
| | | 3H), 2.32-2.14 (m, 2H), 1.99 (s, | |
| | | 3H). | |
| 386 | 751.2 | ¹H NMR (500 MHz, Chloroform-d) δ | 5 |
| | | 8.25 (d, J = 2.0 Hz, 1H), 8.20 (d, J = | |
| | | 2.2 Hz, 1H), 7.91 (d, J = 1.9 Hz, 1H), | |
| | | 7.73-7.72 (m, 1H), 7.60-7.52-7.44 | |
| | | (m, 5H), 7.10-6.99 (m, 4H), 4.35- | |
| | | 4.21 (m, 4H), 4.05-4.01 (m, 1H), | |
| | | 3.90-3.78 (m, 2H), 2.99 (s, 2H), | |
| | | 2.26-2.15 (m, 2H). | |
| 387 | 751.2 | ¹H NMR (500 MHz, Chloroform-d) δ | 5 |
| | | 8.25 (d, J = 2.0 Hz, 1H), 8.20 (d, J = | |
| | | 2.2 Hz, 1H), 7.91 (d, J = 1.9 Hz, 1H), | |
| | | 7.73-7.72 (m, 1H), 7.60-7.52-7.44 | |
| | | (m, 5H), 7.10-6.99 (m, 4H), 4.35- | |
| | | 4.21 (m, 4H), 4.05-4.01 (m, 1H), | |
| | | 3.90-3.78 (m, 2H), 2.99 (s, 2H), | |
| | | 2.26-2.15 (m, 2H). | |
| 388 | 787.2 | ¹H NMR (500 MHz, Chloroform-d) δ | 5 |
| | | 8.02 (d, J = 1.9 Hz, 1H), 7.93 (d, J = | |
| | | 2.2 Hz, 1H), 7.87-7.85 (m, 2H), | |
| | | 7.58-7.55 (m, 1H), 7.38-7.32 (m, | |
| | | 2H), 7.16-7.13 (m, 2H), 6.97-6.91 | |
| | | (m, 2H), 6.21-6.16 (m, 1H), 4.35- | |
| | | 4.30 (m, 1H), 4.17-4.11 (m, 2H), | |
| | | 3.92-3.83 (m, 2H), 3.85-3.74 (m, | |
| | | 2H), 3.39-3.33 (m, 2H), 2.95 (s, 3H), | |
| | | 2.90-2.79 (m, 4H), 2.33-2.30 (m, | |
| | | 1H), 2.27-2.22 (m, 1H). | |
| 389 | 787.2 | ¹H NMR (500 MHz, Chloroform-d) δ | 5 |
| | | 8.02 (d, J = 1.9 Hz, 1H), 7.93 (d, J = | |
| | | 2.2 Hz, 1H), 7.87-7.85 (m, 2H), | |
| | | 7.58-7.55 (m, 1H), 7.38-7.32 (m, | |
| | | 2H), 7.16-7.13 (m, 2H), 6.97-6.91 | |
| | | (m, 2H), 6.21-6.16 (m, 1H), 4.35- | |
| | | 4.30 (m, 1H), 4.17-4.11 (m, 2H), | |
| | | 3.92-3.83 (m, 2H), 3.85-2.74 (m, | |
| | | 2H), 3.39-3.33 (m, 2H), 2.95 (s, 3H), | |
| | | 2.90-2.79 (m, 4H), 2.33-2.30 (m, | |
| | | 1H), 2.27-2.22 (m, 1H). | |
| 390 | 705.3 | ¹H NMR (500 MHz, Chloroform-d) δ | 3 |
| | | 10.65 (s, 1H), 9.63 (d, J = 7.3 Hz, | |
| | | 1H), 8.32 (d, J = 2.2 Hz, 1H), 7.90 (d, | |
| | | J = 2.1 Hz, 1H), 7.82 (d, J = 2.1 Hz, | |
| | | 1H), 7.51 (d, J = 8.7 Hz, 1H), 7.49- | |
| | | 7.45 (m, 4H), 7.21-7.13 (m, 2H), | |
| | | 7.13 (d, J = 7.6 Hz, 1H), 6.80-6.55 | |
| | | (m, 2H), 5.32-5.24 (m, 2H), 4.33- | |
| | | 4.28 (m, 2H), 4.05-3.93 (m, 3H), | |
| | | 3.83-3.76 (m, 2H), 3.34-2.85 (m, | |
| | | 6H), 2.67-2.17 (m, 5H). | |
| 391 | 675.3 | ¹H NMR (500 MHz, Chloroform-d) δ | 3 |
| | | 12.04 (s, 1H), 10.57 (s, 1H), 8.53 (d, | |
| | | J = 2.0 Hz, 1H), 8.40 (d, J = 1.4 Hz, | |
| | | 1H), 7.94 (d, J = 2.1 Hz, 1H), 7.90 (d, | |
| | | J = 7.9 Hz, 1H), 7.81 (d, J = 7.9 Hz, | |
| | | 1H), 7.55-7.50 (m, 3H), 7.46-7.44 | |
| | | (m, 2H), 7.35 (d, J = 8.1 Hz, 1H), | |
| | | 6.95-6.92 (m, 2H), 5.26 (s, 2H), 4.33- | |
| | | 4.28 (m, 2H), 4.05-3.93 (m, 3H), | |
| | | 3.80-3.76 (m, 2H), 3.32-2.97 (m, | |
| | | 4H), 2.91-2.89 (m, 5H), 2.30-2.15 | |
| | | (m, 2H). | |
| 392 | 688.3 | ¹H NMR (500 MHz, Chloroform-d) δ | 3 |
| | | 11.08 (s, 1H), 9.96 (s, 1H), 8.59 (d, J = | |
| | | 2.0 Hz, 1H), 8.17-8.13 (m, 1H), | |
| | | 7.93 (d, J = 2.1 Hz, 1H), 7.90 (d, J = | |
| | | 7.3 Hz, 1H), 7.55-7.50 (m, 3H), 7.45- | |
| | | 7.44 (m, 2H), 7.36-7.32 (m, 2H), | |
| | | 6.80-6.55 (m, 2H), 5.24 (s, 2H), 4.33- | |
| | | 4.28 (m, 2H), 4.06-3.85 (m, 4H), | |
| | | 3.56-3.32 (m, 3H), 3.09-2.89 (m, | |
| | | 4H), 2.33-2.24 (m, 8H). | |
| 393 | 677.2 | ¹H NMR (500 MHz, Chloroform-d) δ | 3 |
| | | 9.99 (s, 1H), 9.01 (d, J = 6.6 Hz, 1H), | |
| | | 8.05-7.96 (m, 2H), 7.83 (d, J = | |
| | | 7.3 Hz, 1H), 7.54 (d, J = 8.7 Hz, 1H), | |
| | | 7.45-7.42 (m, 3H), 7.39-7.34 (m, | |
| | | 2H), 7.21-7.13 (m, 3H), 6.84-6.70 | |
| | | (m, 2H), 6.55-6.51 (m, 1H), 5.32- | |
| | | 5.24 (m, 2H), 4.32-4.28 (m, 2H), | |
| | | 4.03-3.76 (m, 5H), 3.32-2.89 (m, | |
| | | 8H). | |
| 394 | 691.3 | ¹H NMR (500 MHz, Chloroform-d) δ | 3 |
| | | 10.90 (s, 1H), 9.53 (d, J = 6.6 Hz, | |
| | | 1H), 8.06-7.96 (m, 2H), 7.82 (d, J = | |
| | | 7.3 Hz, 1H), 7.58 (d, J = 8.7 Hz, 1H), | |
| | | 7.48-7.43 (m, 3H), 7.40-7.34 (m, | |
| | | 2H), 7.21-7.13 (m, 3H), 6.79-6.70 | |
| | | (m, 1H), 6.53-6.51 (m, 1H), 5.39- | |
| | | 5.24 (m, 2H), 4.30-4.22 (m, 5H), | |
| | | 4.01-3.76 (m, 5H), 3.31-2.89 (m, | |
| | | 8H). | |
| 395 | 720.2 | ¹H NMR (500 MHz, Chloroform-d) δ | 3 |
| | | 12.19 (s, 1H), 10.68 (s, 1H), 8.64 (d, | |
| | | J = 1.4 Hz, 1H), 7.97 (d, J = 2.1 Hz, | |
| | | 1H), 7.90 (d, J = 7.8 Hz, 1H), 7.69 (d, | |
| | | J = 7.9 Hz, 1H), 7.53 (d, J = 8.7 Hz, | |
| | | 1H), 7.51-7.48 (m, 4H), 7.21-7.13 | |
| | | (m, 2H), 6.80-6.55 (m, 2H), 5.31- | |
| | | 5.24 (m, 2H), 4.33-4.28 (m, 2H), | |
| | | 4.05-3.94 (m, 2H), 3.95-3.76 (m, | |
| | | 3H), 3.30-2.89 (m, 9H), 2.32-2.15 | |
| | | (m, 2H). | |
| 396 | 651.2 | ¹H NMR (500 MHz, Chloroform-d) δ | 3 |
| | | 10.91 (s, 1H), 8.41 (d, J = 1.7 Hz, | |
| | | 1H), 8.34 (d, J = 1.7 Hz, 1H), 7.74 (d, | |
| | | J = 1.7 Hz, 1H), 7.71 (d, J = 3.7 Hz, | |
| | | 1H), 7.43-7.33 (m, 5H), 7.17-7.16 | |
| | | (m, 2H), 6.85-6.80 (m, 2H), 6.57- | |
| | | 6.53 (m, 1H), 5.39-5.36 (m, 2H), | |
| | | 4.33-4.28 (m, 2H), 4.17-4.16 (m, | |
| | | 1H), 4.00-3.93 (m, 1H), 3.81-3.76 | |
| | | (m, 2H), 3.32-3.29 (m, 2H), 3.08- | |
| | | 3.02 (m, 1H), 3.02-2.97 (m, 1H), | |
| | | 2.89-2.81 (m, 2H), 2.32-2.15 (m, | |
| | | 2H). | |
| 397 | 665.3 | ¹H NMR (500 MHz, Chloroform-d) δ | 3 |
| | | 8.49 (d, J = 1.7 Hz, 1H), 8.23 (d, J = | |
| | | 1.7 Hz, 1H), 7.85 (d, J = 1.7 Hz, 1H), | |
| | | 7.46-7.44 (m, 1H), 7.45 (t, J = 7.9 | |
| | | Hz, 1H), 7.42-7.38 (m, 3H), 7.20- | |
| | | 7.16 (m, 2H), 6.87-6.84 (m, 1H), | |
| | | 6.58-6.51 (m, 2H), 5.39-5.34 (m, | |
| | | 2H), 4.39-4.28 (m, 2H), 4.16-4.14 | |
| | | (m, 1H), 4.05-3.95 (m, 2H), 3.83- | |
| | | 3.76 (m, 2H), 3.32-2.97 (m, 4H), | |
| | | 2.89-2.80 (m, 2H), 2.32-2.15 (m, 2H). | |
| 398 | 720.2 | ¹H NMR (500 MHz, Chloroform-d) δ | 3 |
| | | 8.89 (s, 1H), 8.17-8.09 (m, 2H), | |
| | | 7.53-7.47 (m, 2H), 7.44-7.35 (m, | |
| | | 3H), 7.26-7.15 (m, 2H), 6.85-6.75 | |
| | | (m, 2H), 5.45-5.34 (m, 2H), 4.26- | |
| | | 4.23 (m, 2H), 4.16-4.13 (m, 1H), | |
| | | 4.05-3.93 (m, 2H), 3.86-3.76 (m, | |
| | | 2H), 3.34-2.85 (m, 6H), 2.33-2.15 | |
| | | (m, 2H). | |
| 399 | 720.2 | ¹H NMR (500 MHz, Chloroform-d) δ | 3 |
| | | 8.85 (s, 1H), 8.43 (d, J = 8.0 Hz, 1H), | |
| | | 8.21-8.09 (m, 2H), 7.50-7.45 (m, | |
| | | 2H), 7.41-7.34 (m, 2H), 7.20-7.16 | |
| | | (m, 2H), 6.85 (d, J = 7.8 Hz, 1H), | |
| | | 6.53-6.52 (m, 1H), 5.33-5.26 (m, | |
| | | 2H), 4.36-4.29 (m, 1H), 4.28-4.25 | |
| | | (m, 1H), 4.20-3.93 (m, 3H), 3.85- | |
| | | 3.76 (m, 2H), 3.34-2.85 (m, 6H), 2.33- | |
| | | 2.15 (m, 2H). | |
| 400 | 667.2 | ¹H NMR (500 MHz, Chloroform-d) δ | 2 |
| | | 11.59 (s, 1H), 8.15 (d, J = 2.1 Hz, | |
| | | 1H), 7.85 (d, J = 8.7 Hz, 1H), 7.46- | |
| | | 7.37 (m, 4H), 7.25 (s, 1H), 7.15-7.07 | |
| | | (m, 3H), 6.62-6.52 (m, 2H), 5.33- | |
| | | 5.24 (m, 2H), 5.04-4.95 (m, 2H), | |
| | | 4.37 (dd, J = 15.5, 5.3 Hz, 1H), 4.29- | |
| | | 4.21 (m, 2H), 4.00-3.93 (m, 2H), | |
| | | 3.88-3.76 (m, 2H), 3.34-2.85 (m, | |
| | | 6H), 2.22-2.15 (m, 2H). | |
| 401 | 667.2 | ¹H NMR (500 MHz, Chloroform-d) δ | 2 |
| | | 9.95 (s, 1H), 8.65 (s, 1H), 8.05 (d, J = | |
| | | 2.1 Hz, 1H), 7.80-7.79 (m, 2H), 7.42- | |
| | | 7.37 (m, 3H), 7.32-7.17 (m, 3H), | |
| | | 6.85-6.55 (m, 2H), 6.20 (d, J = 6.8 | |
| | | Hz, 1H), 5.36-5.24 (m, 2H), 4.45- | |
| | | 4.38 (m, 2H), 4.27 (d, J = 15.6 Hz, | |
| | | 1H), 4.03-3.90 (m, 3H), 3.76-3.71 | |
| | | (m, 1H), 3.34-2.85 (m, 6H), 2.31- | |
| | | 2.15 (m, 2H). | |
| 402 | 683.2 | ¹H NMR (500 MHz, Chloroform-d) δ | 2 |
| | | 8.40 (d, J = 5.9 Hz, 1H), 8.01 (d, J = | |
| | | 2.1 Hz, 1H), 7.85-7.78 (m, 2H), 7.50- | |
| | | 7.45 (m, 2H), 7.42-7.39 (m, 3H), | |
| | | 7.19 (d, J = 17.6 Hz, 2H), 6.87-6.79 | |
| | | (m, 2H), 6.53-6.50 (m, 1H), 5.35- | |
| | | 5.24 (m, 2H), 4.38 (d, J = 15.5, 1H), | |
| | | 4.29-4.17 (m, 2H), 4.00 (d, J = 13.7 | |
| | | Hz, 1H), 3.93-3.85 (m, 2H), 3.76- | |
| | | 3.70 (m, 1H), 3.34-2.85 (m, 6H), 2.31- | |
| | | 2.15 (m, 2H). | |
| 403 | 685.2 | ¹H NMR (500 MHz, Chloroform-d) δ | 2 |
| | | 8.15 (d, J = 2.1 Hz, 1H), 7.93 (d, J = | |
| | | 4.9 Hz, 1H), 7.87 (m, 1H), 7.43 (t, J = | |
| | | 7.9 Hz, 1H), 7.40-7.36 (m, 2H), | |
| | | 7.30 (dd, J = 8.1, 1.2 Hz, 1H), 7.16- | |
| | | 7.12 (m, 2H), 6.87-6.55 (m, 2H), 5.31- | |
| | | 5.24 (m, 2H), 4.30-4.17 (m, 3H), | |
| | | 4.02-3.95 (m, 2H), 3.88-3.79 (m, | |
| | | 2H), 3.33-3.02 (m, 6H), 2.30-2.11 | |
| | | (m, 2H). | |
| 404 | 668.2 | ¹H NMR (500 MHz, Chloroform-d) δ | 2 |
| | | 8.04 (d, J = 2.1 Hz, 1H), 7.70-7.59 | |
| | | (m, 4H), 7.42-7.34 (m, 3H), 7.17- | |
| | | 7.12 (m, 2H), 6.84 (m, 1H), 6.51-6.34 | |
| | | (m, 2H), 5.35-5.31 (m, 2H), 4.35- | |
| | | 4.28 (m, 2H), 4.18-4.13 (m, 1H), | |
| | | 4.04-3.93 (m, 1H), 3.80-3.76 (m, | |
| | | 1H), 3.34-2.89 (m, 6H), 2.31-2.15 | |
| | | (m, 2H). | |
| 405 | 712.0 | ¹H NMR (500 MHz, Chloroform-d) δ | 2 |
| | | 7.62-7.55 (m, 2H), 7.40-7.37 (m, | |
| | | 3H), 7.18-7.15 (m, 2H), 6.81 (d, J = | |
| | | 7.8 Hz, 1H), 6.52-6.51 (m, 1H), 5.31- | |
| | | 5.24 (m, 2H), 4.31-4.27 (m, 2H), | |
| | | 4.02-3.95 m, 2H), 3.90-3.79 (m, | |
| | | 2H), 3.70-3.44 (m, 2H), 3.33- | |
| | | 3.28 (m, 1H), 3.00-2.69 (m, 8H), | |
| | | 2.32-2.04 (m, 2H). | |
| 406 | 699.2 | ¹H NMR (500 MHz, Chloroform-d) δ | 2 |
| | | 8.54 (d, J = 2.0 Hz, 1H), 7.84 (d, J = | |
| | | 2.2 Hz, 1H), 7.63-7.59 (m, 2H), | |
| | | 7.50-7.44 (m, 4H), 6.85-6.75 (m, | |
| | | 2H), 5.55 (s, 2H), 4.34-4.30 (m, 2H), | |
| | | 4.05-3.79 (m, 5H), 3.44-3.34 (m, | |
| | | 2H), 3.20-2.97 (m, 7H), 2.34-2.04 | |
| | | (m, 2H). | |
| 407 | 762.2 | ¹H NMR (500 MHz, Chloroform-d) δ | 1 |
| | | 13.41 (s, 1H), 10.55 (s, 1H), 8.55 (d, | |
| | | J = 2.0 Hz, 1H), 8.21 (d, J = 2.2 Hz, | |
| | | 1H), 7.98 (d, J = 1.9 Hz, 1H), 7.71- | |
| | | 7.68 (m, 2H), 7.55-7.54 (m, 2H), 7.49- | |
| | | 7.42 (m, 4H), 7.22 (d, J = 8.1 Hz, | |
| | | 2H), 7.01 (d, J = 1.7 Hz, 1H), 6.75- | |
| | | 6.74 (m, 1H), 5.33 (s, 2H), 5.26 (s, | |
| | | 2H), 4.24 (s, 2H), 3.87-3.86 (m, 2H), | |
| | | 3.10 (s, 3H), 1.43 (d, J = 5.9 Hz, 3H). | |
| 408 | 748.3 | ¹H NMR (500 MHz, Chloroform-d) δ | 2 |
| | | 13.43 (s, 1H), 10.55 (s, 1H), 8.21 (d, | |
| | | J = 2.2 Hz, 1H), 7.98 (d, J = 1.9 Hz, | |
| | | 1H), 7.71-7.69 (m, 1H), 7.52-7.48 | |
| | | (m, 4H), 7.42-7.37 (m, 2H), 7.17- | |
| | | 7.13 (m, 2H), 7.01 (d, J = 1.7 Hz, | |
| | | 1H), 6.86 (dd, J = 7.8, 1.2 Hz, 1H), | |
| | | 6.52-6.50 (m, 1H), 5.30-5.28 (m, | |
| | | 4H), 4.00 (s, 2H), 3.87-3.86 (m, 2H), | |
| | | 3.30-3.29 (m, 2H), 3.06-3.02 (m, | |
| | | 2H), 2.90-2.85 (m, 5H), 1.36 (t, J = | |
| | | 5.8 Hz, 3H). | |
| 409 | 789.2 | ¹H NMR (500 MHz, Chloroform-d) δ | 1, 5 |
| | | 13.33 (s, 1H), 10.45 (s, 1H), 8.38 (d, | |
| | | J = 2.0 Hz, 1H), 8.27 (d, J = 2.2 Hz, | |
| | | 1H), 7.98 (d, J = 1.9 Hz, 1H), 7.70- | |
| | | 7.66 (m, 2H), 7.49-7.47 (m, 3H), | |
| | | 7.44-7.42 (m, 2H), 7.09 (t, J = 7.6 | |
| | | Hz, 1H), 7.02-6.98 (m, 4H), 5.30 (s, | |
| | | 2H), 4.24 (d, J = 2.4 Hz, 2H), 3.89- | |
| | | 3.87 (m, 2H), 3.10 (s, 3H), 1.34 (t, J = | |
| | | 5.8 Hz, 3H). | |
| 410 | 758.3 | ¹H NMR (500 MHz, Chloroform-d) δ | 5 |
| | | 12.59 (s, 2H), 10.58 (s, 2H), 8.33 (d, | |
| | | J = 2.0 Hz, 2H), 8.20 (d, J = 2.2 Hz, | |
| | | 2H), 7.98 (d, J = 1.9 Hz, 2H), 7.70- | |
| | | 7.66 (m, 2H), 7.52-7.49 (m, 4H), | |
| | | 7.44-7.42 (m, 4H), 7.06 (dd, J = | |
| | | 7.7, 2.3 Hz, 2H), 7.01-6.96 (m, 4H), | |
| | | 6.94 (s, 1H), 6.05-6.03 (m, 2H), 5.31 | |
| | | (d, J = 0.7 Hz, 4H), 3.92-3.88 (m, | |
| | | 8H), 3.33-3.30 (m, 4H), 3.11 (s, 6H), | |
| | | 2.83-2.80-2.71 (m, 8H), 1.33 (t, J = | |
| | | 5.8 Hz, 6H). | |
| 411 | 789.2 | ¹H NMR (500 MHz, Chloroform-d) δ | 1, 5 |
| | | 12.97 (s, 1H), 10.53 (s, 1H), 8.38 (d, | |
| | | J = 2.0 Hz, 1H), 8.23 (d, J = 2.2 Hz, | |
| | | 1H), 7.98 (d, J = 1.9 Hz, 1H), 7.73 | |
| | | (dd, J = 8.1, 2.2 Hz, 1H), 7.61 (d, J = | |
| | | 7.9 Hz, 1H), 7.49-7.47 (m, 3H), | |
| | | 7.44-7.42 (m, 2H), 7.13 (t, J = 7.6 | |
| | | Hz, 1H), 7.02-6.99 (m, 4H), 5.32 (s, | |
| | | 2H), 4.24 (d, J = 2.4 Hz, 2H), 3.86- | |
| | | 3.83 (m, 2H), 3.10 (s, 3H), 1.34 (t, J = | |
| | | 5.8 Hz, 3H). | |
| 412 | 789.2 | ¹H NMR (500 MHz, Chloroform-d) δ | 1, 5 |
| | | 12.97 (s, 1H), 10.54 (s, 1H), 8.38 (d, | |
| | | J = 2.0 Hz, 1H), 8.23 (d, J = 2.2 Hz, | |
| | | 1H), 7.98 (d, J = 1.9 Hz, 1H), 7.73 | |
| | | (dd, J = 8.1, 2.2 Hz, 1H), 7.61 (d, J = | |
| | | 7.9 Hz, 1H), 7.49-7.47 (m, 3H), | |
| | | 7.44-7.42 (m, 2H), 7.13 (t, J = 7.6 | |
| | | Hz, 1H), 7.02-6.98 (m, 4H), 5.32 (s, | |
| | | 2H), 4.24 (d, J = 2.4 Hz, 2H), 3.86- | |
| | | 3.83 (m, 2H), 3.11 (s, 3H), 1.34 (t, J = | |
| | | 5.8 Hz, 3H). | |
| 413 | 741.2 | ¹H NMR (500 MHz, Chloroform-d) δ | 1, 5 |
| | | 10.32 (s, 1H), 8.20 (d, J = 2.4 Hz, | |
| | | 1H), 7.92 (d, J = 2.0 Hz, 1H), 7.75- | |
| | | 7.73 (m, 1H), 7.60-7.58 (m, 1H), | |
| | | 7.56-7.55 (m, 1H), 7.45-7.42 (m, | |
| | | 3H), 7.33-7.32 (m, 1H), 7.21-7.19 | |
| | | (m, 2H), 7.10-7.08 (m, 1H), 6.47- | |
| | | 6.46 (m, 1H), 5.32-5.29 (m, 2H), | |
| | | 4.30-4.28 (m, 2H), 4.13-4.10 (m, | |
| | | 2H), 3.93-3.87 (m, 2H), 3.73-3.71 | |
| | | (m, 1H), 2.95 (s, 2H), 2.21-2.18 (m, | |
| | | 3H). | |
| 414 | 779.2 | ¹H NMR (500 MHz, Chloroform-d) δ | 1, 5 |
| | | 10.36 (s, 1H), 8.20 (d, J = 2.4 Hz, | |
| | | 1H), 7.89 (d, J = 2.0 Hz, 1H), 7.75- | |
| | | 7.74 (m, 1H), 7.60-7.59 (m, 1H), | |
| | | 7.52-7.46 (m, 6H), 7.19-7.13 (m, | |
| | | 3H), 7.02 (d, J = 1.7 Hz, 1H), 6.47- | |
| | | 6.46 (m, 1H), 5.31 (s, 2H), 5.29 (d, J = | |
| | | 1.0 Hz, 2H), 4.10 (d, J = 1.0 Hz, | |
| | | 2H), 3.85-3.82 (m, 2H), 2.92 (s, 3H), | |
| | | 1.29 (d, J = 6.0 Hz, 2H). | |
| 415 | 779.2 | ¹H NMR (500 MHz, Chloroform-d) δ | 1, 5 |
| | | 10.36 (s, 1H), 8.20 (d, J = 2.2 Hz, | |
| | | 1H), 7.89 (d, J = 2.0 Hz, 1H), 7.75- | |
| | | 7.74 (m, 1H), 7.62-7.60 (m, 1H), 7.52- | |
| | | 7.47 (m, 4H), 7.41-7.38 (m, 1H), | |
| | | 7.31-7.26 (m, 2H), 7.18-7.14 (m, | |
| | | 2H), 7.01 (d, J = 1.7 Hz, 1H), 6.73- | |
| | | 6.71 (m, 1H), 5.31-5.29 (m, 4H), | |
| | | 4.20 (s, 2H), 3.85-3.82 (m, 2H), 2.95 | |
| | | (s, 3H), 1.29 (d, J = 5.9 Hz, 2H). | |
| 416 | 753.2 | ¹H NMR (500 MHz, Chloroform-d) δ | 1, 5 |
| | | 11.34 (s, 1H), 10.09 (s, 1H), 8.22 (d, | |
| | | J = 2.2 Hz, 1H), 7.91 (d, J = 1.9 Hz, | |
| | | 1H), 7.73-7.71 (m, 1H), 7.60-7.56 | |
| | | (m, 3H), 7.50-7.47 (m, 2H), 7.33- | |
| | | 7.30 (m, 1H), 7.10-7.06 (m, 3H), 6.68 | |
| | | (dd, J = 7.8, 1.2 Hz, 1H), 5.33-5.29 | |
| | | (m, 2H), 4.72-4.71 (m, 2H), 4.32- | |
| | | 4.39 (m, 2H), 4.11-4.08 (m, 2H), | |
| | | 3.95-3.92 (m, 1H), 3.81-3.77 (m, | |
| | | 2H), 3.73-3.71 (m, 1H), 2.93 (s, | |
| | | 3H), 2.25-2.19 (m, 2H). | |
| 417 | 757.2 | ¹H NMR (500 MHz, Chloroform-d) δ | 1, 5 |
| | | 11.37 (s, 1H), 10.15 (s, 1H), 8.20 (d, | |
| | | J = 2.2 Hz, 1H), 8.00 (d, J = 1.9 Hz, | |
| | | 1H), 7.70-7.66 (m, 3H), 7.50-7.47 | |
| | | (m, 3H), 7.38-7.35 (m, 1H), 7.21- | |
| | | 7.18 (m, 3H), 6.70-6.68 (m, 1H), 5.32- | |
| | | 5.29 (m, 2H), 4.60-4.57 (m, 2H), | |
| | | 4.30-4.28 (m, 1H), 4.10-4.06 (m, | |
| | | 2H), 3.89-3.88 (m, 2H), 3.72-3.69 | |
| | | (m, 2H), 2.94 (s, 3H). | |

### Example 2. Evaluation of the In Vitro Bioactivity

A. GLP-1R cAMP Assay

1. Materials

(1) Reagents

cAMP Detection Kit, Cisbio (Cat #62AM4PEJ)

1M HEPES, Invitrogen (Cat #15630-106)

1×HBSS, Invitrogen (Cat #14025)

BSA, Sigma (Cat #B2064-100G)

IBMX, Sigma (Cat #I5879)

GLP-1(7-37), Hao Yuan (HY-P0055)

(2) Culture Medium

The cell line was constructed by WuXi. The detailed information was showed in the below table.

| Target | Host cell line | Clone |
|---|---|---|
| GLP-1 | HEK293 | N/A |

(3) Apparatus

OptiPlate-384, White, PerkinElmer (Cat #6007290);

384 well plate for Echo, Labcyte (Cat #P-05525);

EnVision, PerkinElmer;

Vi-cell counter, Beckman (Cat #Vi-CELL™ XR Cell Viability Analyzer)

2. Methods
a) The compound source plate preparation:
The reference 7-37 was diluted from 500 nM, 4-fold, 10 points in DMSO using Bravo. The compounds were diluted from 10000000 nM, 3-fold, 10 points in DMSO using Bravo.
b) Transfer of compounds:
1) Use Echo transfer 100 nL cpd to the OptiPlate-384 plate.
2) Spin the plates at 1000 rpm for 5 sec.
c) Cell suspension preparation:
1) Thaw 1 vial of GLP-1 cells rapidly in 37° C. water bath.
2) Transfer cell suspension into 10 ml of HBSS in a 15 ml conical tube.
3) Centrifuge cells at 1000 rpm for 5 min at room temperature to pellet cells
4) Gently aspirate supernatant being careful not to aspirate cells.
5) Flick pellet to loose cells and re-suspend the cell pellet in 10 ml HBSS. Use a sterile pipette to pipette up and down to dislodge clumps.
6) Count cells for concentration and to determine cell viability on Vi-cell.
7) Re-suspend the GLP-1 cells in assay buffer at a concentration of 1.0×E5/mL cells.
8) Transfer 10 μL GLP-1 cells to OptiPlate-384 plate.
d) HTRF cAMP Assay for agonist:
1) Incubate the plate at RT for 30 min before adding the detection reagent.
2) Add 10 μL each of detection reagent using Electric Multiple Channel Pipette.
3) Cover the 384 plate with TopSeal-A film and incubate for 60 minutes at room temperature.
Remove the TopSeal-A, read on EnVision.

3. Date Analysis

A. Standard Curve:

Create a new log (Inhibitor) vs response—Variable slope mode in Prism5.0 software, substitute the standard curve concentration (x) and its corresponding 665/615 data (y) into the model, convert the standard curve concentration (abscissa) by x=log(x), and make the standard curve through the converted xy.

B. cAMP Level Conversion:

Transform the data of 665/615 in the whole plate into a column of data, substitute it into y in the table of standard curve concentration (x) and 665/615 data (y), copy the x (interpolatedx) corresponding to automatic conversion by the software into a table, convert it into cAMP level by the formula x=10\hat{ }(x), and reset the data into 384 distribution.

C. % Activity

% Activity=(cAMP level−LC)/(HC−LC)*100%
Z factor: 1-3*(STD_HC+STD_HC)/(HC-LC)
Assay Window: HC/LC
LC: The average cAMP level of adding DMSO wells
HC: The average cAMP level of adding the top concentration of reference compounds wells.

D. Sample Curve:

Create a new log (slope) vs response—Variable agonist mode in Prism5.0 software, substitute the sample concentration (x) and its corresponding % Activity data (y) into the model, convert the standard curve concentration (abscissa) to x=log(x), and make the sample curve through the converted xy.

Table 2 shows the biological activity of compounds in GLP-1R agonist cAMP stimulation Assay

TABLE 2
The biological activity of compounds in
GLP-1R agonist cAMP stimulation Assay

| Example | EC50(nM) |
|---|---|
| 1 | 4152 |
| 2 | 0.32 |
| 3 | 0.56 |
| 4 | 0.14 |
| 5 | 0.12 |
| 6 | 1.21 |
| 7 | 0.35 |
| 8 | 1.33 |
| 9 | 2982 |
| 10 | 993 |
| 11 | 567.4 |
| 12 | 121.7 |
| 13 | 189.2 |
| 14 | 447.4 |
| 15 | 25.65 |
| 16 | 145.7 |
| 17 | 403.9 |
| 18 | 411 |
| 19 | 175.8 |
| 20 | 3239 |
| 21 | 160.3 |
| 22 | 1369 |
| 23 | >10000 |
| 24 | 90.06 |
| 25 | 756.5 |
| 26 | >10000 |
| 27 | 128.8 |
| 28 | 75.69 |
| 29 | >10000 |
| 30 | 1383 |
| 31 | 37.23 |
| 32 | 111.7 |
| 33 | 61 |
| 34 | 90.06 |
| 35 | >10000 |
| 36 | >10000 |
| 37 | 106.1 |
| 38 | 17.68 |
| 39 | 285.7 |
| 40 | 36.45 |
| 41 | 376.3 |
| 42 | >10000 |
| 43 | 256.1 |
| 44 | 4.36 |
| 45 | 1.39 |
| 46 | 42.42 |
| 47 | 9.6 |
| 48 | 4.36 |
| 49 | 2.47 |
| 50 | 34.05 |
| 51 | 133.5 |
| 52 | 12.78 |
| 53 | 114.2 |
| 54 | 79.72 |
| 55 | 1.83 |
| 56 | 0.97 |
| 57 | 1.41 |
| 58 | 4.35 |
| 59 | 7.2 |
| 60 | 22.28 |
| 61 | 6.75 |
| 62 | 0.51 |
| 63 | 0.49 |
| 64 | 150.8 |
| 65 | 1.75 |
| 66 | 1.41 |
| 67 | 5.89 |
| 68 | 142.5 |
| 69 | 70.47 |
| 70 | 1.9 |
| 71 | 132.1 |
| 72 | 0.61 |
| 73 | 0.52 |
| 74 | >10000 |
| 75 | >10000 |
| 76 | 9.04 |
| 77 | 1.9 |
| 78 | 23.11 |
| 79 | 76.45 |
| 80 | 2.05 |
| 81 | 14.5 |
| 82 | 115.9 |
| 83 | 3.76 |
| 84 | 17.95 |
| 85 | 0.41 |
| 86 | 0.75 |
| 87 | 10.05 |
| 88 | 4.45 |
| 89 | 87.84 |
| 90 | 1.59 |
| 91 | 26.16 |
| 92 | 18.65 |
| 93 | 29.81 |
| 94 | 371.7 |
| 95 | 62 |
| 96 | 74.34 |
| 97 | 139.6 |
| 98 | 16.94 |
| 99 | 42.42 |
| 100 | 0.98 |
| 101 | 0.75 |
| 102 | 1.33 |
| 103 | 0.83 |
| 104 | 1.05 |
| 105 | 0.78 |
| 106 | 2.31 |
| 107 | 1.83 |
| 108 | 2.39 |
| 109 | 46.97 |
| 110 | 4.45 |
| 111 | 2.34 |
| 112 | 6.18 |
| 113 | 4.38 |
| 114 | 1.04 |
| 115 | 0.87 |
| 116 | 0.92 |
| 117 | 10.21 |
| 118 | 8.31 |
| 119 | 0.63 |
| 120 | 15.32 |
| 121 | 4.23 |
| 122 | 3.21 |
| 123 | 5.49 |
| 124 | 0.17 |
| 125 | 0.55 |
| 126 | 0.18 |
| 127 | 0.15 |
| 128 | 0.3 |
| 129 | 1.14 |
| 130 | 1.34 |
| 131 | 1.21 |
| 132 | 0.74 |
| 133 | 0.38 |
| 134 | 0.85 |
| 135 | 1.25 |
| 136 | 2.19 |
| 137 | 5.49 |
| 138 | 8.92 |
| 139 | 15.23 |
| 140 | 2.97 |
| 141 | 3.29 |
| 142 | 0.21 |
| 143 | 0.75 |
| 144 | 23.36 |
| 145 | 0.45 |
| 146 | 23.98 |
| 147 | 0.68 |
| 148 | 21.07 |
| 149 | 34.65 |
| 150 | 5.78 |
| 151 | 0.6 |
| 152 | 0.64 |
| 153 | 0.59 |
| 154 | 0.45 |
| 155 | 0.55 |
| 156 | 0.68 |
| 157 | 0.77 |
| 158 | 1.03 |
| 159 | 9.74 |
| 160 | 0.98 |
| 161 | 0.87 |
| 162 | 1.23 |
| 163 | 0.99 |
| 164 | 1.68 |
| 165 | 0.88 |
| 166 | 0.76 |
| 167 | 0.68 |
| 168 | 10.48 |
| 169 | 1.02 |
| 170 | 3.55 |
| 171 | 9.53 |
| 172 | 1.63 |
| 173 | 31.47 |
| 174 | 2.13 |
| 175 | 1.23 |
| 176 | 0.87 |
| 177 | 0.23 |
| 178 | 0.19 |
| 179 | 0.76 |
| 180 | 1.06 |
| 181 | 0.21 |
| 182 | 0.32 |
| 183 | 0.76 |
| 184 | 1.57 |
| 185 | 1.32 |
| 186 | 2.12 |
| 187 | 0.2 |
| 188 | 1.19 |
| 189 | 3.54 |
| 190 | 9.27 |
| 191 | 3.69 |
| 192 | 9.87 |
| 193 | 0.21 |
| 194 | 0.33 |
| 195 | 2.31 |
| 196 | 0.17 |
| 197 | 0.19 |
| 198 | 0.35 |
| 199 | 0.47 |
| 200 | 0.82 |
| 201 | 1.99 |
| 202 | 2.21 |
| 203 | 0.24 |
| 204 | 0.63 |
| 205 | 0.98 |
| 206 | 5.91 |
| 207 | 0.43 |
| 208 | 0.25 |
| 209 | 0.32 |
| 210 | 0.39 |
| 211 | 1.02 |
| 212 | 2.96 |
| 213 | 0.78 |
| 214 | 1.21 |
| 215 | 2.11 |
| 216 | 0.82 |
| 217 | 0.79 |
| 218 | 1.63 |
| 219 | 1.21 |
| 220 | 0.44 |
| 221 | 2.13 |
| 222 | 3.56 |
| 223 | 0.95 |
| 224 | 3.29 |
| 225 | 0.18 |
| 226 | 0.12 |
| 227 | 0.22 |
| 228 | 1.26 |
| 229 | 0.21 |
| 230 | 0.09 |
| 231 | 0.87 |
| 232 | 1.26 |
| 233 | 0.92 |
| 234 | 3.87 |
| 235 | 0.42 |
| 236 | 1.04 |
| 237 | 8.71 |
| 238 | 11.92 |
| 239 | 3.19 |
| 240 | 20.73 |
| 241 | 0.31 |
| 242 | 0.17 |
| 243 | 0.48 |
| 244 | 0.33 |
| 245 | 0.51 |
| 246 | 0.91 |
| 247 | 0.83 |
| 248 | 1.28 |
| 249 | 0.44 |
| 250 | 1.49 |
| 251 | 2.22 |
| 252 | 1.03 |
| 253 | 2.01 |
| 254 | 1.92 |
| 255 | 0.32 |
| 256 | 12.53 |
| 257 | 15.11 |
| 258 | 31.75 |
| 259 | 0.91 |
| 260 | 1.03 |
| 261 | 0.81 |
| 262 | 3.12 |
| 263 | 5.78 |
| 264 | 4.22 |
| 265 | 45.9 |
| 266 | 3.19 |
| 267 | 1.99 |
| 268 | 3.64 |
| 269 | 10.78 |
| 270 | 1.02 |
| 271 | 0.97 |
| 272 | 3.21 |
| 273 | 2.78 |
| 274 | 1.65 |
| 275 | 6.21 |
| 276 | 0.65 |
| 277 | 0.76 |
| 278 | 3.18 |
| 279 | 0.95 |
| 280 | 21.03 |
| 281 | 1.21 |
| 282 | 4.32 |
| 283 | 10.23 |
| 284 | 9.85 |
| 285 | 0.42 |
| 286 | 0.21 |
| 287 | 2.98 |
| 288 | 3.87 |
| 289 | 0.99 |
| 290 | 1.2 |
| 291 | 0.75 |
| 292 | 8.73 |
| 293 | 0.99 |
| 294 | 1.02 |
| 295 | 2.19 |
| 296 | 1.92 |
| 297 | 3.65 |
| 298 | 0.37 |
| 299 | 0.16 |
| 300 | 0.19 |
| 301 | 0.18 |
| 302 | 1.07 |
| 303 | 0.91 |
| 304 | 0.18 |
| 305 | 0.19 |
| 306 | 0.56 |
| 307 | 0.21 |
| 308 | 3.12 |
| 309 | 0.87 |
| 310 | 0.93 |
| 311 | 0.39 |
| 312 | 0.99 |
| 313 | 1.39 |
| 314 | 0.63 |
| 315 | 1.03 |
| 316 | 0.54 |
| 317 | 0.32 |
| 318 | 1.53 |
| 319 | 0.43 |
| 320 | 13.67 |
| 321 | 0.94 |
| 322 | 31.92 |
| 323 | 2.73 |
| 324 | 2.95 |
| 325 | 1.05 |
| 326 | 0.64 |
| 327 | 3.96 |
| 328 | 12.73 |
| 329 | 1.66 |
| 330 | 0.88 |
| 331 | 1.27 |
| 332 | 1.58 |
| 333 | 2.19 |
| 334 | 0.61 |
| 335 | 0.97 |
| 336 | 1.03 |
| 337 | 0.45 |
| 338 | 1.32 |
| 339 | 0.39 |
| 340 | 0.28 |
| 341 | 0.31 |
| 342 | 0.91 |
| 343 | 0.54 |
| 344 | 1.02 |
| 345 | 0.21 |
| 346 | 0.76 |
| 347 | 0.81 |
| 348 | 0.91 |
| 349 | 1.61 |
| 350 | 0.51 |
| 351 | 0.19 |
| 352 | 1.03 |
| 353 | 0.44 |
| 354 | 0.21 |
| 355 | 0.78 |
| 356 | 2.21 |
| 357 | 0.41 |
| 358 | 0.98 |
| 359 | 1.62 |
| 360 | 0.19 |
| 361 | 0.9 |
| 362 | 0.85 |
| 363 | 0.41 |
| 364 | 0.38 |
| 365 | 1.32 |
| 366 | 5.97 |
| 367 | 3.92 |
| 368 | 9.19 |
| 369 | 2.91 |
| 370 | 5.12 |
| 371 | 3.19 |
| 372 | 0.65 |
| 373 | 0.39 |
| 374 | 0.55 |
| 375 | 1.01 |
| 376 | 0.98 |
| 377 | 3.41 |
| 378 | 1.03 |
| 379 | 3.12 |
| 380 | 6.91 |
| 381 | 1.99 |
| 382 | 0.1 |
| 383 | 1.92 |
| 384 | 0.08 |
| 385 | 0.61 |
| 386 | 0.15 |
| 387 | 0.86 |
| 388 | 0.82 |
| 389 | 2.81 |
| 390 | 5.21 |
| 391 | 0.91 |
| 392 | 10.12 |
| 393 | 15.21 |
| 394 | 21.09 |
| 395 | 10.34 |
| 396 | 145.87 |
| 397 | 127.19 |
| 398 | 110.03 |
| 399 | >10000 |
| 400 | 104.39 |
| 401 | 21.83 |
| 402 | 73.19 |
| 403 | 23.19 |
| 404 | 19.76 |
| 405 | 39.87 |
| 406 | 99.17 |
| 407 | 0.16 |
| 408 | 0.21 |
| 409 | 0.42 |
| 410 | 0.31 |
| 411 | 0.33 |
| 412 | 3.1 |
| 413 | 0.49 |
| 414 | 0.57 |
| 415 | 0.71 |
| 416 | 0.37 |
| 417 | 0.65 |

B. Tag-Lite Binding Assay

1. Materials

(1) Reagents

| Reagent | Vendor | Catalog Number | Lot Number |
|---|---|---|---|
| SNAP-Lumi4-Tb-5 nmoles | Cisbio | SSNPTBD | 13A |
| GLP-1 receptors red agonist | Cisbio | L0030RED | 08A |
| Tag-lite labeling medium (5X) | Cisbio | LABMED | 13A |

DMEM, Gibco (Cat #11965-092);

FBS, Excell (Cat #FSP500);

Penicillin/Streptomycin (100×), 10000 units/ml, Hyclone-SV30010;

G418, Gibco (Cat #10131-027);

DPBS, Corning (Cat #21-031-CVC)

TE, Gibco (Cat #25300-062);

(2) Culture Medium

The cell line was constructed by WuXi.

| Host cell line | Target | Media |
|---|---|---|
| HEK293 | GLP-1 | DMEM + 10% FBS + 1% PS + 500G418 |

(3) Apparatus

Vi-cell counter, Beckman (Cat #Vi-CELL™ XR Cell Viability Analyzer);

Incubator, Thermo (1153447);

Microplate, 384 well, PS, F-Bottom, Small volume, Hibase, Med. Binding, White, Greiner (Cat #784075);

EnVision, PerkinElmer (651436);

Bravo V11, Agilent (337076)

ECHO 655, Labcyte (2474218);

2. Methods

First Day: Cell Seeding

1) Remove the culture medium by aspiration.

2) Rinse the cells with DPBS and remove by aspiration

3) Add 3 ml of Trypsin-EDTA (0.05%).

4) Incubate at 37° C. for 1 min. Check the progress of the enzyme treatment with an inverted phase-contrast microscope.

5) Tap the culture dish to detach the cells from the bottom of the dish. Add 10 ml of growth medium, suspend the cells well by pipetting, and wash any remaining cells from the bottom of the dish.

6) Centrifuge cells at 1000 rpm at room temperature for 5 min

7) Gently pour off or aspirate supernatant, being careful not to aspirate cells.

8) Re-suspend the cell pellet in growth media. Use a sterile pipette to pipette up and down to dislodge clumps.

9) Count cells using a Vi-CELL™ to determine cell viability and cell concentration. Prepare a cell suspension in plating media/well.

10) Dispense cells into cell plate (1.8 M cells/well in 2 ml medium of 6-well plate, total 4 wells).

11) Place plates in 37° C./5% CO2 incubator for 16-20 hours.

Second Day: Tag-Lite Binding Assay

1) Thaw the 5 ml vial of Tag-lite buffer (TLB 5×). Dilute 1:5 Tag-lite.

2) Dilute the vials of Tag-lite SNAP-Lumi4-Tb solution, using the 100 μM stock solution and carrying out a 1:1000 dilution.

3) The cell culture medium is removed from the 6-well plate and then 1 mL of Tag-lite SNAP-Lumi4-Tb diluted in Tag-lite labeling medium are added gently on the cells.

4) The cells are incubated for 1 h at 37° C.+5% CO2.

5) The cells are washed twice with 1 mL of Tag-lite buffer.

6) Trypsinize cells and suspend to 1×106 cells/mL in 1×TLB.

7) The testing compounds and reference compound are serially diluted with 3-fold, 10 points using Echo, then transfer 100 nL compounds to the assay plate.

8) Add the labeled cells to each well according to the plate map, 10000 cells/10 uL/well. Centrifuge the plate at 1000 rpm for 1 min.

9) Incubate the plate at 23° C., 30 min.

10) Add 50 nL of red agonist (final conc.: 4.917 nM) to destination plate using Echo.

11) Incubate the plate at 23° C. for 2 h.

12) Read the plate on an HTRF® compatible reader (at 665 nm and 620 nm).

3. Date Analysis

a) % Inhibition:

% Inhibition=100%−(Ratio−LC)/(HC−LC)*100%

Z factor: 1-3*(STD_HC+STD_HC)/(HC-LC)

Assay Window: HC/LC

HC: The average ratio of adding DMSO wells

LC: The average ratio of adding the top concentration of reference compounds wells

b) The “log (inhibitor) vs. response—Variable slope” model in GraphPad Prism 5.0 was used to fit the dose-response curve of each sample. The sample concentration (x) and its corresponding % Inhibition data (y) were substituted into the model. The sample concentration (abscissa) was x=log(x) transformed. The transformed xy was used as the sample curve to obtain the IC50.

c) Calculate the sample Ki according to the sample IC50 and red agonist Kd. S in the formula is the actual used concentration of red agonist.

$$K i = \frac{IC 50}{1 + S / Kd}$$

Table 3 shows the biological activity of compounds in GLP-1R agonist Tag-lite Binding Assay.

TABLE 3
The biological activity of compounds in Tag-lite Binding Assay

| Example | IC50(nM) | Ki |
|---|---|---|
| 2 | 345.2 | 164.55 |
| 3 | 1750 | 876.24 |
| 5 | 1705 | 910.51 |
| 62 | 2632 | 1317.87 |
| 80 | 2325 | 1164.15 |
| 85 | 143.2 | 71.7 |
| 88 | 1838 | 920.31 |
| 90 | 1898 | 950.35 |
| 100 | 1261 | 893.31 |
| 101 | 1281 | 641.41 |
| 103 | 1552 | 885.3 |
| 105 | 1945 | 1004.3 |
| 126 | 1398 | 806.2 |
| 127 | 1252 | 743.5 |
| 134 | 1838 | 903.9 |
| 142 | 2011 | 1001.42 |
| 145 | 1922 | 956.21 |
| 147 | 1749 | 841.2 |
| 151 | 1048 | 519.32 |
| 153 | 1451 | 872.72 |
| 154 | 1582.3 | 904.1 |
| 166 | 2651 | 1324.4 |
| 178 | 875.32 | 429.66 |
| 196 | 152.3 | 87.9 |
| 197 | 165.3 | 84 |
| 203 | 158 | 90.3 |
| 207 | 149 | 82.37 |
| 229 | 121.5 | 75.91 |
| 244 | 189.2 | 110.1 |
| 245 | 146 | 82.3 |
| 276 | 2456 | 1005.6 |
| 334 | 1213 | 692.3 |
| 335 | 1107 | 615.4 |
| 340 | 123 | 88.5 |
| 341 | 158.3 | 95.5 |
| 342 | 140.4 | 72.2 |
| 354 | 2137 | 1057.5 |
| 382 | 689.2 | 341.5 |
| 383 | 1477 | 899.3 |
| 384 | 109.9 | 68.32 |
| 385 | 158.1 | 82.7 |
| 386 | 119.6 | 69.9 |
| 387 | 346.3 | 204.7 |
| 391 | 115.8 | 70.3 |
| 407 | 180.2 | 110.5 |
| 408 | 105.9 | 68.4 |
| 409 | 486.3 | 237.3 |
| 411 | 119.7 | 72.6 |
| 413 | 28.02 | 14.13 |
| 414 | 31.7 | 15.56 |
| 415 | 232.78 | 116.63 |
| 416 | 54.3 | 29.33 |
| 417 | 136.7 | 69.21 |

What is claimed is:

1. A compound of Formula I-A-1: 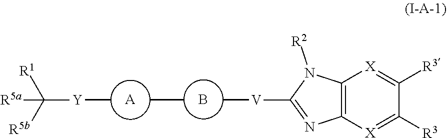
or a pharmaceutically acceptable salt thereof, wherein
R¹ is C₁₋₆ alkyl, C₁₋₆ haloalkyl, C₂₋₆ alkenyl, C₂₋₆ alkynyl, C₃₋₁₀ cycloalkyl, heterocyclyl, C₆₋₁₀ aryl, 5-10 membered heteroaryl, —S—R^{1b}, —S(O)R^{1b}, —S(O)(NH)R^{1b}, —S(O)₂R^{1b}, —S(O)₂N(R^{1b})(R^{1c}), —S(O)(NR^{1b})R^{1c}, —C(O)N(R^{1b})(R^{1c}), —C(O)R^{1b}, or —C(O)OR^{1c}, wherein the alkyl, haloalkyl, cycloalkyl, aryl, 5-10 membered heteroaryl, or heterocyclyl is each optionally substituted with one to four Z¹, and wherein any two Z¹ can be combined with the atoms to which they are attached to form a C₃₋₁₀ cycloalkyl or heterocyclyl or C₆₋₁₀ aryl or 5-10 membered heteroaryl;
Ring A is C₃₋₁₀ cycloalkyl, heterocyclyl, C₆₋₁₀ aryl or 5-10 membered heteroaryl, which is each optionally substituted with one to four Z^{1a};
Ring B is C₃₋₁₀ cycloalkyl, heterocyclyl, C₆₋₁₀ aryl or 5-10 membered heteroaryl, which is each optionally substituted with one to four R⁴;
Ring A and Ring B each contains zero to three double bonds;
R² is H, C₁₋₆ alkyl, —C₁₋₆ alkyl-alkoxy, —C₁₋₆ alkyl-cycloalkoxy, C₁₋₆ haloalkyl, C₂₋₆ alkenyl, C₂₋₆ alkynyl, C₃₋₁₀ cycloalkyl, heterocyclyl, C₆₋₁₀ aryl, 5-10 membered heteroaryl, —CN, —OR^{2a}, —S—R^{2a}, —S(O)R^{2a}, —S(O)(NH)R^{2a}, —S(O)₂R^{2a}, —S(O)₂N(R^{2a})(R^{2b}), or —S(O)(NR^{2a})R^{2b}, wherein the alkyl, alkoxy, cycloalkoxy, haloalkyl, cycloalkyl, heterocyclyl, aryl, or 5-10 membered heteroaryl in each instance is optionally substituted with one to four Z¹, and wherein any two Z₁ can be combined with the atoms to which they are attached to form a C₃₋₁₀ cycloalkyl or heterocyclyl or C₆₋₁₀ aryl or 5-10 membered heteroaryl;
X is each independently —N═, —C(H)═, or —C(R⁸)═;
each R³ and R^{3′} is independently H, C₁₋₆ alkyl, C₁₋₆ haloalkyl, C₂₋₆ alkenyl, C₂₋₆ alkynyl, halogen, C₃₋₁₀ cycloalkyl, heterocyclyl, C₆₋₁₀ aryl, 5-10 membered heteroaryl, —CN, —NO₂, —OR^{3a}, —C(O)R^{3a}, —CH₂C(O)OR^{3a}, —C(O)OR^{3a}, —C(O)N(R^{3a})(R^{3b}), —N(R^{3a})C(O)R^{3b}, —N(R^{3a})C(O)OR^{3b}, —N(R^{3a})C(O)N(R^{3b})₂, —C(O)NHS(O)₂R^{3a}, —C(O)NR^{3a}S(O)₂R^{3b}, —C(O)NR^{3a}S(O)₂NR^{3b}R^{3c}, —C(O)NR^{3a}—S(O)(═NR^{3b})R^{3c}, —S(O)₂R^{3a}, —S(O)₂OR^{3a}, —S(O)₂N(R^{3a})(R^{3b}), —N(R^{3a})S(O)₂R^{3b}, —S(O)₂NHC(O)R^{3a}, —S(O)(═NR^{3a})R^{3b}, —S(O)(═NR^{3a})NR^{3b}, —S(═NR^{3a})(═NR^{3b})R^{3c}, —P(O)(OR^{3a})(R^{3b}), —P(O)(OR^{3a})(OR^{3b}), —B(OR^{3a})(OR^{3b}), or —O—C₁₋₆alkyl-C(O)OR^{3a}, wherein the alkyl, haloalkyl, alkenyl, alkynyl, cycloalkyl, heterocyclyl, aryl, or 5-10 membered heteroaryl in each instance is optionally substituted with one to four R^{3d};
each R^{3a}, R^{3b}, and R^{3c} is independently H, C₁₋₆ alkyl, C₆₋₁₀ aryl, 5-10 membered heteroaryl, C₁₋₆ haloalkyl, C₂₋₈ alkoxyalkyl, —C₁₋₄ alkyl-N(R^{9a})(R^{9b}), —C₁₋₄ alkyl-N(R^{9a})C(O)—O—C₁₋₄ alkyl-OP(O)(OR^{9c})₂, —C₁₋₄ alkyl-C(O)N(R^{9a})(R^{9b}), —C₁₋₄ alkyl-O—C(O)—O—C₁₋₄ alkyl, —C₁₋₄ alkyl-O—C(O)—C₁₋₄ alkyl-N(R^{9a})(R^{9b}), —C₁₋₄ alkyl-O—C(O)—C₁₋₄ alkyl-OP(O)(OR^{9c})₂, —C₁₋₄ alkyl-C₃₋₈ cycloalkyl, —C₁₋₄ alkyl-heterocyclyl, C₂₋₆ alkenyl, C₂₋₆ alkynyl, C₃₋₁₀ cycloalkyl, heterocyclyl, —CH₂CH(N(R^{9a})₂)C(O)OR^{9b}, —P(O)(OR^{9c})₂, —OP(O)(OR^{9c})₂, —CH₂P(O)(OR^{9c})₂, —CH₂OP(O)(OR^{9c})₂, —OCH₂P(O)(OR^{9c})₂, —C(O)OCH₂P(O)(OR^{9c})₂, —P(O)(R^{9c})(OR^{9d}), —OP(O)(R^{9c})(OR^{9d}), —CH₂P(O)(R^{9c})(OR^{9d}), —OCH₂P(O)(R^{9c})(OR^{9d}), —C(O)OCH₂P(O)(R^{9c})(OR^{9d}), —P(O)(N(R^{9c})₂)₂, —OP(O)(N(R^{9c})₂)₂, —CH₂P(O)(N(R^{9c})₂)₂, —OCH₂P(O)(N(R^{9c})₂)₂, —C(O)OCH₂P(O)(N(R^{9c})₂)₂, —P(O)(N(R^{9c})₂)(OR^{9d}), —OP(O)(N(R^{9c})₂)(OR^{9d}), —CH₂P(O)(N(R^{9c})₂)(OR^{9d}), —OCH₂P(O)(N(R^{9c})₂)(OR^{9d}), —C(O)OCH₂P(O)(N(R^{9c})₂)(OR^{9d}), —P(O)(R^{9c})(N(R^{9d})₂), —OP(O)(R^{9c})(N(R^{9d})₂), —CH₂P(O)(R^{9c})(N(R^{9d})₂), —OCH₂P(O)(R^{9c})(N(R^{9d})₂), —C(O)OCH₂P(O)(R^{9c})(N(R^{9d})₂), C₁₋₆ alkyl-heterocyclyl, C₁₋₆ alkyl-aryl, or C₁₋₆ alkyl-(5-10 membered) heteroaryl, wherein the alkyl, alkenyl, cycloalkyl, heterocyclyl, aryl, or 5-10 membered heteroaryl in each instance is optionally substituted with one to four Z^{1b};
each R⁴ is independently H, —OH, CH₂OH, C₁₋₉ alkyl, C₁₋₈ haloalkyl, C₁₋₆ haloalkoxy, C₂₋₆ alkoxyalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —NO₂, —CN, —N₃, —O—R^{4a}, —C(O)R^{4a}, —C(O)O—R^{4a}, —C(O)N(R^{4a})(R^{4b}), —N(R^{4a})(R^{4b}), —N(R^{4a})₂(R^{4b})⁺, —N(R^{4a})—C(O)R^{4b}, —N(R^{4a})C(O)O(R^{4b}), —N(R^{4a})C(O)N(R^{4b})(R^{4c}), —N(R^{4a})S(O)₂(R^{4b}), —N(R^{4a})S(O)₂—N(R^{4b})(R^{4c}), —N(R^{4a})S(O)₂O(R^{4b}), —OC(O)R^{4a}, —OC(O)OR^{4a}, —OC(O)—N(R^{4a})(R^{4b}), —S—R^{4a}, —S(O)R^{4a}, —S(O)(NH)R^{4a}, —S(O)₂R^{4a}, —S(O)₂N(R^{4a})(R^{4b}), —S(O)(NR^{4a})R^{4b}, or —Si(R^{4a})₃, wherein the alkyl, haloalkyl, alkenyl, alkynyl, cycloalkyl, heterocyclyl, aryl, or 5-10 membered heteroaryl in each instance is optionally substituted with one to four Z^{1b}; or two R⁴ groups attached to adjacent ring atoms are combined with the atoms to which they are attached to form a C₅₋₁₀ cycloalkyl or heterocyclyl, which is each optionally substituted with one to four Z^{1b};
each R^{5a} and R^{5b} is independently H, C₁₋₆ alkyl, C₁₋₆ alkyloxy, C₁₋₆ haloalkyl, C₂₋₆ alkoxyalkyl, halogen, C₃₋₁₀ cycloalkyl, heterocyclyl, —C₁₋₆ alkyl-N(R^{9a})(R^{9b}), —CN, —OR^{5a1}, or —N(R^{5a1})(R^{5a2}); or R^{5a} and R^{5b} are combined with the atoms to which they are attached to form a C₃₋₁₀ cycloalkyl or heterocyclyl, which is each optionally substituted with one to four R^{5a3}; or R^{5a} or R^{5b} can be combined with the substituents on Ring A to form a C₅₋₁₀ cycloalkyl or heterocyclyl, which is each optionally substituted with one to four R^{5a3};
each R^{5a1} and R^{5a2} is independently H, C₁₋₆ alkyl, C₂₋₆ alkenyl, C₂₋₆ alkynyl, C₃₋₁₀ cycloalkyl, heterocyclyl, C₆₋₁₀ aryl, or 5-10 membered heteroaryl, wherein the cycloalkyl, heterocyclyl, aryl, or 5-10 membered heteroaryl in each instance is optionally substituted with one to four R^{5a4};
V is —C(O)—, —O—, —N(R^{6a})—, or —C(R^{6b})(R^{6c})—;
R^{6a} is H, C₁₋₆ alkyl, C₃₋₁₀ cycloalkyl, heterocyclyl, —S(O)₂R^{6a1}, or —S(O)₂N(R^{6a1})(R^{6a2}), wherein the cycloalkyl or heterocyclyl in each instance is optionally substituted with C₁₋₆ alkyl, F, or —CN;
each R^{6b} and R^{6c} is independently H, C₁₋₆ alkyl, C₁₋₆ haloalkyl, C₂₋₆ alkoxyalkyl, halogen, C₃₋₁₀ cycloalkyl, heterocyclyl, —C₁₋₆ alkyl-N(R^{9a})(R^{9b}), —CN, —OR^{6c1}, or —N(R^{6c2})(R^{6c3}), wherein the alkyl, cycloalkyl, or heterocyclyl in each instance is optionally substituted with one to four R^{6b1}; or R^{6b} and R^{6c} are combined with the atom to which they are attached to form C₃₋₁₀ cycloalkyl or heterocyclyl, which is each optionally substituted with one to four R^{6b1}; or R^{6a} or R^{6c} is combined with one R⁴ group and the atoms to which they are attached to form a C₅₋₁₀ cycloalkyl or heterocyclyl, which is each optionally substituted with one to four R¹⁰;
Y is —N(R⁷)—, —O—, —S—, —S(O)—, —S(O)₂—, —S(O)(═NH)—, or —S(O)(═NR⁷)—;
each R^{3d}, R^{5a3}, R^{5a4}, R^{6b1}, and R¹⁰ is independently H, C₁₋₆ alkyl, C₁₋₆ haloalkyl, C₁₋₆ alkoxy, C₁₋₆ haloalkoxy, C₂₋₆ alkoxyalkyl, C₂₋₆ alkenyl, C₂₋₆ alkynyl, halogen, C₃₋₁₀ cycloalkyl, heterocyclyl, C₆₋₁₀ aryl, 5-10 membered heteroaryl, oxo, —OH, —CN, CO₂R^{3e}, —NO₂, or —C(O)N(R^{2a})(R^{2b}), wherein the heterocyclyl or 5-10 membered heteroaryl in each instance is optionally substituted with C₁₋₆ alkyl, C₁₋₆ haloalkyl, or C₁₋₆ haloalkoxy;
each R^{6a1}, R^{6a2}, R^{6c1}, R^{6c2}, R^{6c3} and R⁷ is independently H, C₁₋₆ alkyl or C₃₋₁₀ cycloalkyl;
each R⁸ is independently C₁₋₉ alkyl, C₁₋₈ haloalkyl, C₂₋₆ alkenyl, C₂₋₆ alkynyl, halogen, oxo, —OH, —CN, CO₂R^{3e}, —NO₂, —NH₂, —N₃, —SH, —O(C₁₋₉ alkyl), —O(C₁₋₈ haloalkyl), —NH(C₁₋₉ alkyl), —NH(C₁₋₈ haloalkyl), —N(C₁₋₉ alkyl)₂, —N(C₁₋₈ haloalkyl)₂;
each R^{9a} and R^{9b} is independently H, C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —NO₂, —CN, —N₃, —O—R^{12a}, —C(O)R^{12a}, —C(O)O—R^{12a}, —C(O)N(R^{12a})(R^{12b}), —N(R^{12a})(R^{12b}), —N(R^{12a})₂(R^{12b})⁺, —N(R^{12a})—C(O)R^{12b}, —N(R^{12a})C(O)O(R^{12b}), —N(R^{12a})C(O)N(R^{12b})(R^{12c}), —N(R^{12a})S(O)₂(R^{12b}), —N(R^{12a})S(O)₂—N(R^{12b})(R^{12c}), —N(R^{12a})S(O)₂O(R^{12b}), —OC(O)R^{12a}, —OC(O)OR^{12a}, —OC(O)—N(R^{12a})(R^{12b}), —S—R^{12a}, —S(O)R^{12a}, —S(O)(NH)R^{12a}, —S(O)₂R^{12a}, —S(O)₂N(R^{12a})(R^{12b}), —S(O)(NR^{12a})R^{12b}, or —Si(R^{12a})₃;
or R^{9a} and R^{9b} together form a 3-10 membered heterocyclyl optionally substituted with C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, halogen, C₃₋₁₅ cycloalkyl, heterocyclyl, C₆₋₁₀ aryl, heteroaryl, oxo, —NO₂, —CN, —N₃, —O—R^{12a}, —C(O)R^{12a}, —C(O)O—R^{12a}, —C(O)N(R^{12a})(R^{12b}), —N(R^{12a})(R^{12b}), —N(R^{12a})₂(R^{12b})⁺, —N(R^{12a})—C(O)R^{12b}, —N(R^{12a})C(O)O(R^{12b}), —N(R^{12a})C(O)N(R^{12b})(R^{12c}), —N(R^{12a})S(O)₂(R^{12b}), —N(R^{12a})S(O)₂—N(R^{12b})(R^{12c}), —N(R^{12a})S(O)₂O(R^{12b}), —OC(O)R^{12a}, —OC(O)OR^{12a}, —OC(O)—N(R^{12a})(R^{12b}), —S—R^{12a}, —S(O)R^{12a}, —S(O)(NH)R^{12a}, —S(O)₂R^{12a}, —S(O)₂N(R^{12a})(R^{12b}), —S(O)(NR^{12a})R^{12b}, or —Si(R^{12a})₃;
each Z¹ is independently C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —NO₂, —N₃, —CN, —O—R^{12a}, —C(O)—R^{12a}, —C(O)O—R^{12a}, —C(O)—N(R^{12a})(R^{12b}), —N(R^{12a})(R^{12b}), —N(R^{12a})₂(R^{12b})⁺, —N(R^{12a})C(O)—R^{12b}, —N(R^{12a})C(O)O—R^{12b}, —N(R^{12a})C(O)N(R^{12b})(R^{12c}), —N(R^{12a})S(O)₂(R^{12b}), —NR^{12a}S(O)₂N(R^{12b})(R^{12c}), —NR^{12a}S(O)₂O(R^{12b}), —OC(O)R^{12a}, —OC(O)OR^{12a}, —OC(O)—N(R^{12a})(R^{12b}), —S—R^{12a}, —S(O)R^{12a}, —S(O)(NH)R^{12a}, —S(O)₂R^{12a}, —S(O)₂N(R^{12a})(R^{12b}), —S(O)(NR^{12a})R^{12b}, or —Si(R^{12a})₃, wherein the alkyl, haloalkyl, alkenyl, alkynyl, cycloalkyl, heterocyclyl, aryl, or 5-10 membered heteroaryl in each instance is optionally substituted with one to four Z^{1a};
each Z^{1a} is independently H, —OH, C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —NO₂, —CN, —N₃, —O—R^{12a}, —C(O)R^{12a}, —C(O)O—R^{12a}, —C(O)N(R^{12a})(R^{12b}), —N(R^{12a})(R^{12b}), —N(R^{12a})₂(R^{12b})⁺, —N(R^{12a})—C(O)R^{12b}, —N(R^{12a})C(O)O(R^{12b}), —N(R^{12a})C(O)N(R^{12b})(R^{12c}), —N(R^{12a})S(O)₂(R^{12b}), —N(R^{12a})S(O)₂—N(R^{12b})(R^{12c}), —N(R^{12a})S(O)₂O(R^{12b}), —OC(O)R^{12a}, —OC(O)OR^{12a}, —OC(O)—N(R^{12a})(R^{12b}), —S—R^{12a}, —S(O)R^{12a}, —S(O)(NH)R^{12a}, —S(O)₂R^{12a}, —S(O)₂N(R^{12a})(R^{12b}), —S(O)(NR^{12a})R^{12b}, or —Si(R^{12a})₃, wherein the alkyl, haloalkyl, alkenyl, alkynyl, cycloalkyl, heterocyclyl, aryl, or 5-10 membered heteroaryl in each instance is optionally substituted with one to four Z^{1b};
each Z^{1b} is independently H, C₁₋₉ alkyl, C₁₋₈ haloalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —OH, —CN, CO₂R^{3e}, —NO₂, —NH₂, —N₃, —SH, —O(C₁₋₉ alkyl), —O(C₁₋₈ haloalkyl), —O(C₂₋₆ alkenyl), —O(C₂₋₆ alkynyl), —O(C₃₋₁₅ cycloalkyl), —O(heterocyclyl), —O(C₆₋₁₀ aryl), —O(5-10 membered heteroaryl), —NH(C₁₋₉ alkyl), —NH(C₁₋₈ haloalkyl), —NH(C₂₋₆ alkenyl), —NH(C₂₋₆ alkynyl), —NH(C₃₋₁₅ cycloalkyl), —NH(heterocyclyl), —NH(C₆₋₁₀ aryl), —NH(5-10 membered heteroaryl), —N(cyclo-alkyl), —N(C₁₋₉ alkyl)₂, —N(C₁₋₈ haloalkyl)₂, —N(C₂₋₆ alkenyl)₂, —N(C₂₋₆ alkynyl)₂, —N(C₃₋₁₅ cycloalkyl)₂, —N(heterocyclyl)₂, —N(C₆₋₁₀ aryl)₂, —N(5-10 membered heteroaryl)₂, —N(C₁₋₉ alkyl)(C₁₋₈ haloalkyl), —N(C₁₋₉ alkyl)(C₂₋₆ alkenyl), —N(C₁₋₉ alkyl)(C₂₋₆ alkynyl), —N(C₁₋₉ alkyl)(C₃₋₁₅ cycloalkyl), —N(C₁₋₉ alkyl)(heterocyclyl), —N(C₁₋₉ alkyl)(C₆₋₁₀ aryl), —N(C₁₋₉ alkyl)(5-10 membered heteroaryl), —C(O)(C₁₋₉ alkyl), —C(O)(C₁₋₈ haloalkyl), —C(O)(C₂₋₆ alkenyl), —C(O)(C₂₋₆ alkynyl), —C(O)(C₃₋₁₅ cycloalkyl), —C(O)(heterocyclyl), —C(O)(C₆₋₁₀ aryl), —C(O)(5-10 membered heteroaryl), —C(O)O(C₁₋₉ alkyl), —C(O)O(C₁₋₈ haloalkyl), —C(O)O(C₂₋₆ alkenyl), —C(O)O(C₂₋₆ alkynyl), —C(O)O(C₃₋₁₅ cycloalkyl), —C(O)O(heterocyclyl), —C(O)O(C₆₋₁₀ aryl), —C(O)O(5-10 membered heteroaryl), —C(O)NH₂, —C(O)NH(C₁₋₉ alkyl), —C(O)NH(C₁₋₈ haloalkyl), —C(O)NH(C₂₋₆ alkenyl), —C(O)NH(C₂₋₆ alkynyl), —C(O)NH(C₃₋₁₅ cycloalkyl), —C(O)NH(heterocyclyl), —C(O)NH(C₆₋₁₀ aryl), —C(O)NH(5-10 membered heteroaryl), —C(O)N(C₁₋₉ alkyl)₂, —C(O)N(C₁₋₈ haloalkyl)₂, —C(O)N(C₂₋₆ alkenyl)₂, —C(O)N(C₂₋₆ alkynyl)₂, —C(O)N(C₃₋₁₅ cycloalkyl)₂, —C(O)N(heterocyclyl)₂, —C(O)N(C₆₋₁₀ aryl)₂, —C(O)N(5-10 membered heteroaryl)₂, —NHC(O)(C₁₋₉ alkyl), —NHC(O)(C₁₋₈ haloalkyl), —NHC(O)(C₂₋₆ alkenyl), —NHC(O)(C₂₋₆ alkynyl), —NHC(O)(C₃₋₁₅ cycloalkyl), —NHC(O)(heterocyclyl), —NHC(O)(C₆₋₁₀ aryl), —NHC(O)(5-10 membered heteroaryl), —NHC(O)O(C₁₋₉ alkyl), —NHC(O)O(C₁₋₈ haloalkyl), —NHC(O)O(C₂₋₆ alkenyl), —NHC(O)O(C₂₋₆ alkynyl), —NHC(O)O(C₃₋₁₅ cycloalkyl), —NHC(O)O(heterocyclyl), —NHC(O)O(C₆₋₁₀ aryl), —NHC(O)O(5-10 membered heteroaryl), —NHC(O)NH(C₁₋₉ alkyl), —NHC(O)NH(C₁₋₈ haloalkyl), —NHC(O)NH(C₂₋₆ alkenyl), —NHC(O)NH(C₂₋₆ alkynyl), —NHC(O)NH(C₃₋₁₅ cycloalkyl), —NHC(O)NH(heterocyclyl), —NHC(O)NH(C₆₋₁₀ aryl), —NHC(O)NH(5-10 membered heteroaryl), —NHS(O)(C₁₋₉ alkyl), —N(C₁₋₉ alkyl)(S(O)(C₁₋₉ alkyl), —S(C₁₋₉ alkyl), —S(C₁₋₈ haloalkyl), —S(C₂₋₆ alkenyl), —S(C₂₋₆ alkynyl), —S(C₃₋₁₅ cycloalkyl), —S(heterocyclyl), —S(C₆₋₁₀ aryl), —S(5-10 membered heteroaryl), —S(O)N(C₁₋₉ alkyl)₂, —S(O)(C₁₋₉ alkyl), —S(O)(C₁₋₈ haloalkyl), —S(O)(C₂₋₆ alkenyl), —S(O)(C₂₋₆ alkynyl), —S(O)(C₃₋₁₅ cycloalkyl), —S(O)(heterocyclyl), —S(O)(C₆₋₁₀ aryl), —S(O)(5-10 membered heteroaryl), —S(O)₂(C₁₋₉ alkyl), —S(O)₂(C₁₋₈ haloalkyl), —S(O)₂(C₂₋₆ alkenyl), —S(O)₂(C₂₋₆ alkynyl), —S(O)₂(C₃₋₁₅ cycloalkyl), —S(O)₂(heterocyclyl), —S(O)₂(C₆₋₁₀ aryl), —S(O)₂(5-10 membered heteroaryl), —S(O)(NH)(C₁₋₉ alkyl), —S(O)₂NH(C₁₋₉ alkyl), or —S(O)₂N(C₁₋₉ alkyl)₂, wherein the alkyl, cycloalkyl, heterocyclyl, aryl, or 5-10 membered heteroaryl in each instance is optionally substituted with one to three C₁₋₉ alkyl, C₁₋₈ haloalkyl, halogen, —OH, —NH₂, CO₂H, —O(C₁₋₉ alkyl), —O(C₁₋₈ haloalkyl), —O(C₃₋₁₅ cycloalkyl), —O(heterocyclyl), —O(aryl), —O(5-10 membered heteroaryl), —NH(C₁₋₉ alkyl), —NH(C₁₋₈ haloalkyl), —NH(C₃₋₁₅ cycloalkyl), —NH(heterocyclyl), —NH(aryl), —NH(5-10 membered heteroaryl), —N(C₁₋₉ alkyl)₂, —N(C₃₋₁₅ cycloalkyl)₂, —NHC(O)(C₁₋₈ haloalkyl), —NHC(O)(C₃₋₁₅ cycloalkyl), —NHC(O)(heterocyclyl), —NHC(O)(aryl), —NHC(O)(5-10 membered heteroaryl), —NHC(O)O(C₁₋₉ alkyl), —NHC(O)O(C₁₋₈ haloalkyl), —NHC(O)O(C₂₋₆ alkynyl), —NHC(O)O(C₃₋₁₅ cycloalkyl), —NHC(O)O(heterocyclyl), —NHC(O)O(aryl), —NHC(O)O(5-10 membered heteroaryl), —NHC(O)NH(C₁₋₉ alkyl), S(O)₂(C₁₋₉ alkyl), —S(O)₂(C₁₋₈ haloalkyl), —S(O)₂(C₃₋₁₅ cycloalkyl), —S(O)₂(heterocyclyl), —S(O)₂(aryl), —S(O)₂(5-10 membered heteroaryl), —S(O)(NH)(C₁₋₉ alkyl), —S(O)₂NH(C₁₋₉ alkyl), or —S(O)₂N(C₁₋₉ alkyl)₂;
each R^{1b}, R^{1c}, R^{2a}, R^{2b}, R^{4a}, R^{4b}, R^{4c}, R^{9c}, R^{9d}, R^{12a}, R^{12b}, and R^{12c} is independently H, —OH, C₁₋₉ alkyl, C₂₋₆ alkenyl, C₂₋₆ alkynyl, C₃₋₁₅ cycloalkyl, heterocyclyl, C₆₋₁₀ aryl, or 5-10 membered heteroaryl, wherein the alkyl, alkenyl, alkynyl, cycloalkyl, heterocyclyl, aryl, or 5-10 membered heteroaryl in each instance is optionally substituted with one to four Z^{1b}; and
each R^{3e} is independently H, C₁₋₆ alkyl, C₁₋₆ haloalkyl, —C₁₋₄ alkyl-N(R^{9a})(R^{9b}), —C₁₋₄ alkyl-C(O)N(R^{9a})(R^{9b}), —C₁₋₄ alkyl-O—C(O)—O—C₁₋₄ alkyl, —C₁₋₄ alkyl-O—C(O)—C₁₋₄ alkyl-N(R^{9a})(R^{9b}), —C₁₋₄ alkyl-C₃₋₈ cycloalkyl, —C₁₋₄ alkyl-heterocyclyl, C₃₋₁₀ cycloalkyl, heterocyclyl, C₆₋₁₀ aryl, 5-10 membered heteroaryl, —P(O)(OR^{9c})₂, —CH₂P(O)(OR^{9c})₂, —OCH₂P(O)(OR^{9c})₂, —C(O)OCH₂P(O)(OR^{9c})₂, —P(O)(R^{9c})(OR^{9d}), —OP(O)(R^{9c})(OR^{9d}), —CH₂P(O)(R^{9c})(OR^{9d}), —C(O)OCH₂P(O)(R^{9c})(OR^{9d}), —P(O)(N(R^{9c})₂)₂, —CH₂P(O)(N(R^{9c})₂)₂, —C(O)OCH₂P(O)(N(R^{9c})₂)₂, —P(O)(N(R^{9c})₂)(OR^{9d}), —CH₂P(O)(N(R^{9c})₂)(OR^{9d}), —C(O)OCH₂P(O)(N(R^{9c})₂)(OR^{9d}), —P(O)(R^{9c})(N(R^{9d})₂), —CH₂P(O)(R^{9c})(N(R^{9d})₂), or —C(O)OCH₂P(O)(R^{9c})(N(R^{9d})₂), wherein the alkyl, alkenyl, cycloalkyl, heterocyclyl, aryl, or 5-10 membered heteroaryl in each instance is optionally substituted with one to four Z^{1b}; wherein each 5-10 membered heteroaryl has 5 to 12 ring members and has one to four heteroatoms, each is independently N, O, or S; and wherein each heterocyclyl has 3 to 12 ring members and has one to four heteroatoms, each is independently N, O, or S.

2. The compound of claim 1, wherein the compound is a compound of Formula I-A-2 or Formula I-A-3 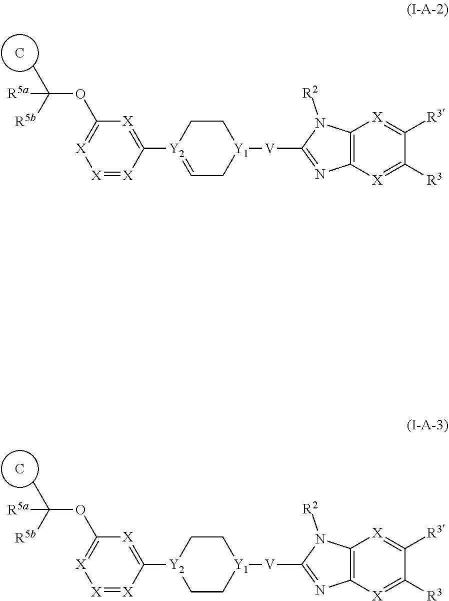
or a pharmaceutically acceptable salt thereof, wherein
Ring C is C₃₋₁₀ cycloalkyl, heterocyclyl, C₆₋₁₀ aryl or 5-10 membered heteroaryl, each is optionally substituted with one to four Z¹;
Y₁—V is N—CH₂, N—CH(CH₃), N—CF₂, or CH—CH₂, CH—CF₂, CH—NH, N—S(O), N—S(O)₂, N—C(O), CH—C(O) or cyclopropyl;
Y₂ is C, CH or N;
R² is H, C₁₋₆ alkyl, C₁₋₆ alkyl-alkoxy, —C₁₋₆ alkyl-cycloalkoxy, C₁₋₆ haloalkyl, C₂₋₆ alkenyl, C₂₋₆ alkynyl, C₃₋₁₀ cycloalkyl, heterocyclyl, C₆₋₁₀ aryl, 5-10 membered heteroaryl, —CN, —OR^{2a}, —S—R^{2a}, —S(O)R^{2a}, —S(O)(NH)R^{2a}, —S(O)₂R^{2a}, —S(O)₂N(R^{2a})(R^{2b}), or —S(O)(NR^{2a})R^{2b}, wherein the alkyl, alkoxy, cycloalkoxy, haloalkyl, cycloalkyl, heterocyclyl, aryl, or 5-10 membered heteroaryl in each instance is optionally substituted with one to four Z¹ and wherein any two Z₁ can be combined with the atoms to which they are attached to form a C₃₋₁₀ cycloalkyl or heterocyclyl or C₆₋₁₀ aryl or 5-10 membered heteroaryl;
X is each independently —N═, —C(H)═, or —C(R⁸)═;
each R³ and R^{3′} is independently H, C₁₋₆ alkyl, C₁₋₆ haloalkyl, C₂₋₆ alkenyl, C₂₋₆ alkynyl, halogen, C₃₋₁₀ cycloalkyl, heterocyclyl, C₆₋₁₀ aryl, 5-10 membered heteroaryl, —CN, —NO₂, —OR^{3a}, —C(O)R^{3a}, —CH₂C(O)OR^{3a}, —C(O)OR^{3a}, —C(O)N(R^{3a})(R^{3b}), —N(R^{3a})C(O)R^{3b}, —N(R^{3a})C(O)OR^{3b}, —N(R^{3a})C(O)N(R^{3b})₂, —C(O)NHS(O)₂R^{3a}, —C(O)NR^{3a}S(O)₂R^{3b}, —C(O)NR^{3a}S(O)₂NR^{3b}R^{3c}, —C(O)NR^{3a}—S(O)(═NR^{3b})R^{3c}, —S(O)₂R^{3a}, —S(O)₂OR^{3a}, —S(O)₂N(R^{3a})(R^{3b}), —N(R^{3a})S(O)₂R^{3b}, —S(O)₂NHC(O)R^{3a}, —S(O)(═NR^{3a})R^{3b}, —S(O)(═NR^{3a})NR^{3b}, —S(═NR^{3a})(═NR^{3b})R^{3c}, —P(O)(OR^{3a})(R^{3b}), —P(O)(OR^{3a})(OR^{3b}), —B(OR^{3a})(OR^{3b}), or —O—C₁₋₆alkyl-C(O)OR^{3a}, wherein the alkyl, haloalkyl, alkenyl, alkynyl, cycloalkyl, heterocyclyl, aryl, or 5-10 membered heteroaryl in each instance is optionally substituted with one to four R^{3d};
each R^{3a}, R^{3b}, and R^{3c} is independently H, C₁₋₆ alkyl, C₆₋₁₀ aryl, 5-10 membered heteroaryl, C₁₋₆ haloalkyl, C₂₋₈ alkoxyalkyl, —C₁₋₄ alkyl-N(R^{9a})(R^{9b}), —C₁₋₄ alkyl-N(R^{9a})C(O)—O—C₁₋₄ alkyl-OP(O)(OR^{9c})₂, —C₁₋₄ alkyl-C(O)N(R^{9a})(R^{9b}), —C₁₋₄ alkyl-O—C(O)—O—C₁₋₄ alkyl, —C₁₋₄ alkyl-O—C(O)—C₁₋₄ alkyl-N(R^{9a})(R^{9b}), —C₁₋₄ alkyl-O—C(O)—C₁₋₄ alkyl-OP(O)(OR^{9c})₂, —C₁₋₄ alkyl-C₃₋₈ cycloalkyl, —C₁₋₄ alkyl-heterocyclyl, C₂₋₆ alkenyl, C₂₋₆ alkynyl, C₃₋₁₀ cycloalkyl, heterocyclyl, —CH₂CH(N(R^{9a})₂)C(O)OR^{9b}, C₁₋₆ alkyl-heterocyclyl, C₁₋₆ alkyl-aryl or C₁₋₆ alkyl-(5-10 membered) heteroaryl, wherein the alkyl, alkenyl, cycloalkyl, heterocyclyl, aryl, or 5-10 membered heteroaryl in each instance is optionally substituted with one to four Z^{1b};
each R^{5a} and R^{5b} is independently H, C₁₋₆ alkyl, C₁₋₆ alkyloxy, C₁₋₆ haloalkyl, C₂₋₆ alkoxyalkyl, halogen, C₃₋₁₀ cycloalkyl, heterocyclyl, —C₁₋₆ alkyl-N(R^{9a})(R^{9b}), —CN, —OR^{5a1}, or —N(R^{5a1})(R^{5a2}); or R^{5a} and R^{5b} are combined with the atoms to which they are attached to form a C₃₋₁₀ cycloalkyl or heterocyclyl, which is each optionally substituted with one to four R^{5a3}; each R^{5a1} and R^{5a2} is independently H, C₁₋₆ alkyl, C₂₋₆ alkenyl, C₂₋₆ alkynyl, C₃₋₁₀ cycloalkyl, heterocyclyl, C₆₋₁₀ aryl, or 5-10 membered heteroaryl, herein the cycloalkyl, heterocyclyl, aryl, or 5-10 membered heteroaryl in each instance is optionally substituted with one to four halogen;
each R^{3d} and R^{5a3} is independently H, C₁₋₆ alkyl, C₁₋₆ haloalkyl, C₁₋₆ alkoxy, C₁₋₆ haloalkoxy, C₂₋₆ alkoxyalkyl, C₂₋₆ alkenyl, C₂₋₆ alkynyl, halogen, C₃₋₁₀ cycloalkyl, heterocyclyl, C₆₋₁₀ aryl, 5-10 membered heteroaryl, oxo, —OH, —CN, CO₂R^{3e}, —NO₂, or —C(O)N(R^{2a})(R^{2b}) wherein the heterocyclyl or 5-10 membered heteroaryl in each instance is optionally substituted with C₁₋₆ alkyl, C₁₋₆ haloalkyl, or C₁₋₆ haloalkoxy;
each R⁸ is independently C₁₋₉ alkyl, C₁₋₈ haloalkyl, C₂₋₆ alkenyl, C₂₋₆ alkynyl, halogen, oxo, —OH, —CN, CO₂R^{3e}, —NO₂, —NH₂, —N₃, —SH, —O(C₁₋₉ alkyl), —O(C₁₋₈ haloalkyl), —NH(C₁₋₉ alkyl), —NH(C₁₋₈ haloalkyl), —N(C₁₋₉ alkyl)₂, —N(C₁₋₈ haloalkyl)₂;
each R^{9a} and R^{9b} is independently H, C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —NO₂, —CN, —N₃, —O—R^{12a}, —C(O)R^{12a}, —C(O)O—R^{12a}, —C(O)N(R^{12a})(R^{12b}), —N(R^{12a})(R^{12b}), —N(R^{12a})₂(R^{12b})⁺, —N(R^{12a})—C(O)R^{12b}, —N(R^{12a})C(O)O(R^{12b}), —N(R^{12a})C(O)N(R^{12b})(R^{12c}), —N(R^{12a})S(O)₂(R^{12b}), —N(R^{12a})S(O)₂—N(R^{12b})(R^{12c}), —N(R^{12a})S(O)₂O(R^{12b}), —OC(O)R^{12a}, —OC(O)OR^{12a}, —OC(O)—N(R^{12a})(R^{12b}), —S—R^{12a}, —S(O)R^{12a}, —S(O)(NH)R^{12a}, —S(O)₂R^{12a}, —S(O)₂N(R^{12a})(R^{12b}), —S(O)(NR^{12a})R^{12b}, or —Si(R^{12a})₃; or R^{9a} and R^{9b} together form a 3-10 membered heterocyclyl optionally substituted with C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, halogen, C₃₋₁₅ cycloalkyl, heterocyclyl, C₆₋₁₀ aryl, heteroaryl, oxo, —NO₂, —CN, —N₃, —O—R^{12a}, —C(O)R^{12a}, —C(O)O—R^{12a}, —C(O)N(R^{12a})(R^{12b}), —N(R^{12a})(R^{12b}), —N(R^{12a})₂(R^{12b})⁺, —N(R^{12a})—C(O)R^{12b}, —N(R^{12a})C(O)O(R^{12b}), —N(R^{12a})C(O)N(R^{12b})(R^{12c}), —N(R^{12a})S(O)₂(R^{12b}), —N(R^{12a})S(O)₂—N(R^{12b})(R^{12c}), —N(R^{12a})S(O)₂O(R^{12b}), —OC(O)R^{12a}, —OC(O)OR^{12a}, —OC(O)—N(R^{12a})(R^{12b}), —S—R^{12a}, —S(O)R^{12a}, —S(O)(NH)R^{12a}, —S(O)₂R^{12a}, —S(O)₂N(R^{12a})(R^{12b}), —S(O)(NR^{12a})R^{12b}, or —Si(R^{12a})₃;
each Z¹ is independently C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —NO₂, —N₃, —CN, —O—R^{12a}, wherein the alkyl, haloalkyl, alkenyl, alkynyl, cycloalkyl, heterocyclyl, aryl, or 5-10 membered heteroaryl in each instance is optionally substituted with one to four Z^{1a};
each Z^{1a} is independently H, C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —NO₂, —CN, —N₃, —O—R^{12a}, —C(O)R^{12a}, —C(O)O—R^{12a}, —C(O)N(R^{12a})(R^{12b}), —N(R^{12a})(R^{12b}), —N(R^{12a})₂(R^{12b})⁺, —N(R^{12a})—C(O)R^{12b}, —N(R^{12a})C(O)O(R^{12b}), —N(R^{12a})C(O)N(R^{12b})(R^{12c}), —N(R^{12a})S(O)₂(R^{12b}), —N(R^{12a})S(O)₂—N(R^{12b})(R^{12c}), —N(R^{12a})S(O)₂O(R^{12b}), —OC(O)R^{12a}, —OC(O)OR^{12a}, —OC(O)—N(R^{12a})(R^{12b}), —S—R^{12a}, —S(O)R^{12a}, —S(O)(NH)R^{12a}, —S(O)₂R^{12a}, —S(O)₂N(R^{12a})(R^{12b}), —S(O)(NR^{12a})R^{12b}, or —Si(R^{12a})₃, wherein the alkyl, haloalkyl, alkenyl, alkynyl, cycloalkyl, heterocyclyl, aryl, or 5-10 membered heteroaryl in each instance is optionally substituted with one to four Z^{1b};
each Z^{1b} is independently H, C₁₋₉ alkyl, C₁₋₈ haloalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —OH, —CN, CO₂R^{3e}, —NO₂, —NH₂, —N₃, —SH, —O(C₁₋₉ alkyl), —O(C₁₋₈ haloalkyl), —O(C₂₋₆ alkenyl), —O(C₂₋₆ alkynyl), —O(C₃₋₁₅ cycloalkyl), —O(heterocyclyl), —O(C₆₋₁₀ aryl), —O(5-10 membered heteroaryl), —NH(C₁₋₉ alkyl), —NH(C₁₋₈ haloalkyl), —NH(C₂₋₆ alkenyl), —NH(C₂₋₆ alkynyl), —NH(C₃₋₁₅ cycloalkyl), —NH(heterocyclyl), —NH(C₆₋₁₀ aryl), —NH(5-10 membered heteroaryl), —N(cyclo-alkyl), —N(C₁₋₉ alkyl)₂, —N(C₁₋₈ haloalkyl)₂, —N(C₂₋₆ alkenyl)₂, —N(C₂₋₆ alkynyl)₂, —N(C₃₋₁₅ cycloalkyl)₂, —N(heterocyclyl)₂, —N(C₆₋₁₀ aryl)₂, —N(5-10 membered heteroaryl)₂, —N(C₁₋₉ alkyl)(C₁₋₈ haloalkyl), —N(C₁₋₉ alkyl)(C₂₋₆ alkenyl), —N(C₁₋₉ alkyl)(C₂₋₆ alkynyl), —N(C₁₋₉ alkyl)(C₃₋₁₅ cycloalkyl), —N(C₁₋₉ alkyl)(heterocyclyl), —N(C₁₋₉ alkyl)(C₆₋₁₀ aryl), —N(C₁₋₉ alkyl)(5-10 membered heteroaryl), —C(O)(C₁₋₉ alkyl), —C(O)(C₁₋₈ haloalkyl), —C(O)(C₂₋₆ alkenyl), —C(O)(C₂₋₆ alkynyl), —C(O)(C₃₋₁₅ cycloalkyl), —C(O)(heterocyclyl), —C(O)(C₆₋₁₀ aryl), —C(O)(5-10 membered heteroaryl), —C(O)O(C₁₋₉ alkyl), —C(O)O(C₁₋₈ haloalkyl), —C(O)O(C₂₋₆ alkenyl), —C(O)O(C₂₋₆ alkynyl), —C(O)O(C₃₋₁₅ cycloalkyl), —C(O)O(heterocyclyl), —C(O)O(C₆₋₁₀ aryl), —C(O)O(5-10 membered heteroaryl), —C(O)NH₂, —C(O)NH(C₁₋₉ alkyl), —C(O)NH(C₁₋₈ haloalkyl), —C(O)NH(C₂₋₆ alkenyl), —C(O)NH(C₂₋₆ alkynyl), —C(O)NH(C₃₋₁₅ cycloalkyl), —C(O)NH(heterocyclyl), —C(O)NH(C₆₋₁₀ aryl), —C(O)NH(5-10 membered heteroaryl), —C(O)N(C₁₋₉ alkyl)₂, —C(O)N(C₁₋₈ haloalkyl)₂, —C(O)N(C₂₋₆ alkenyl)₂, —C(O)N(C₂₋₆ alkynyl)₂, —C(O)N(C₃₋₁₅ cycloalkyl)₂, —C(O)N(heterocyclyl)₂, —C(O)N(C₆₋₁₀ aryl)₂, —C(O)N(5-10 membered heteroaryl)₂, —NHC(O)(C₁₋₉ alkyl), —NHC(O)(C₁₋₈ haloalkyl), —NHC(O)(C₂₋₆ alkenyl), —NHC(O)(C₂₋₆ alkynyl), —NHC(O)(C₃₋₁₅ cycloalkyl), —NHC(O)(heterocyclyl), —NHC(O)(C₆₋₁₀ aryl), —NHC(O)(5-10 membered heteroaryl), —NHC(O)O(C₁₋₉ alkyl), —NHC(O)O(C₁₋₈ haloalkyl), —NHC(O)O(C₂₋₆ alkenyl), —NHC(O)O(C₂₋₆ alkynyl), —NHC(O)O(C₃₋₁₅ cycloalkyl), —NHC(O)O(heterocyclyl), —NHC(O)O(C₆₋₁₀ aryl), —NHC(O)O(5-10 membered heteroaryl), —NHC(O)NH(C₁₋₉ alkyl), —NHC(O)NH(C₁₋₈ haloalkyl), —NHC(O)NH(C₂₋₆ alkenyl), —NHC(O)NH(C₂₋₆ alkynyl), —NHC(O)NH(C₃₋₁₅ cycloalkyl), —NHC(O)NH(heterocyclyl), —NHC(O)NH(C₆₋₁₀ aryl), or —NHC(O)NH(5-10 membered heteroaryl), wherein the alkyl, cycloalkyl, heterocyclyl, aryl, or 5-10 membered heteroaryl in each instance is optionally substituted with one to three C₁₋₉ alkyl, C₁₋₈ haloalkyl, halogen, —OH, —NH₂, CO₂H, —O(C₁₋₉ alkyl), —O(C₁₋₈ haloalkyl), —O(C₃₋₁₅ cycloalkyl), —O(heterocyclyl), —O(aryl), —O(5-10 membered heteroaryl), —NH(C₁₋₉ alkyl), —NH(C₁₋₈ haloalkyl), —NH(C₃₋₁₅ cycloalkyl), —NH(heterocyclyl), —NH(aryl), —NH(5-10 membered heteroaryl), —N(C₁₋₉ alkyl)₂, —N(C₃₋₁₅ cycloalkyl)₂, —NHC(O)(C₁₋₈ haloalkyl), —NHC(O)(C₃₋₁₅ cycloalkyl), —NHC(O)(heterocyclyl), —NHC(O)(aryl), —NHC(O)(5-10 membered heteroaryl), —NHC(O)O(C₁₋₉ alkyl), —NHC(O)O(C₁₋₈ haloalkyl), —NHC(O)O(C₂₋₆ alkynyl), —NHC(O)O(C₃₋₁₅ cycloalkyl), —NHC(O)O(heterocyclyl), —NHC(O)O(aryl), —NHC(O)O(5-10 membered heteroaryl), or —NHC(O)NH(C₁₋₉ alkyl);
each R^{1b}, R^{1c}, R^{2a}, R^{2b}, R^{4c}, R^{9c}, R^{12a}, R^{12b}, and R^{12c} is independently H, —OH, C₁₋₉ alkyl, C₂₋₆ alkenyl, C₂₋₆ alkynyl, C₃₋₁₅ cycloalkyl, heterocyclyl, C₆₋₁₀ aryl, or 5-10 membered heteroaryl, wherein the alkyl, alkenyl, alkynyl, cycloalkyl, heterocyclyl, aryl, or 5-10 membered heteroaryl in each instance is optionally substituted with one to four Z^{1b}; and
each R^{3e} is independently H, C₁₋₆ alkyl, C₁₋₆ haloalkyl, —C₁₋₄ alkyl-N(R^{9a})(R^{9b}), —C₁₋₄ alkyl-C(O)N(R^{9a})(R^{9b}), —C₁₋₄ alkyl-O—C(O)—O—C₁₋₄ alkyl, —C₁₋₄ alkyl-O—C(O)—C₁₋₄ alkyl-N(R^{9a})(R^{9b}), —C₁₋₄ alkyl-C₃₋₈ cycloalkyl, —C₁₋₄ alkyl-heterocyclyl, C₃₋₁₀ cycloalkyl, heterocyclyl, C₆₋₁₀ aryl, or 5-10 membered heteroaryl, wherein the alkyl, alkenyl, cycloalkyl, heterocyclyl, aryl, or 5-10 membered heteroaryl in each instance is optionally substituted with one to four Z^{1b}; wherein each 5-10 membered heteroaryl has 5 to 12 ring members and has one to four heteroatoms, each is independently N, O, or S; and wherein each heterocyclyl has 3 to 12 ring members and has one to four heteroatoms, each is independently N, O, or S.

3. The compound of claim 1, wherein the compound is a compound of Formula I-A-4 or Formula I-A-5 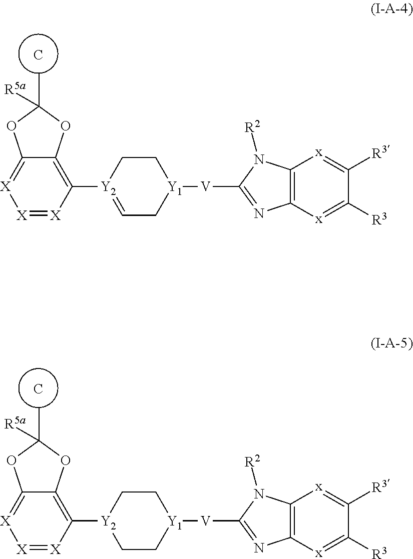
or a pharmaceutically acceptable salt thereof, wherein
Ring C is C₃₋₁₀ cycloalkyl, heterocyclyl, C₆₋₁₀ aryl or 5-10 membered heteroaryl, which is each optionally substituted with one to four Z¹;
Y₁—V is N—CH₂, N—CH(CH₃), N—CF₂, CH—CH₂, CH—CF₂, CH—NH, N—S(O), N—S(O)₂, N—C(O), CH—C(O) or cyclopropyl;
Y₂ is C, CH or N;
R² is H, C₁₋₆ alkyl, C₁₋₆ alkyl-alkoxy, C₁₋₆ alkyl-cycloalkoxy, C₁₋₆ haloalkyl, C₂₋₆ alkenyl, C₂₋₆ alkynyl, C₃₋₁₀ cycloalkyl, heterocyclyl, C₆₋₁₀ aryl, 5-10 membered heteroaryl, —CN, —OR^{2a}, —S—R^{2a}, —S(O)R^{2a}, —S(O)(NH)R^{2a}, —S(O)₂R^{2a}, —S(O)₂N(R^{2a})(R^{2b}), or —S(O)(NR^{2a})R^{2b}, wherein the alkyl, alkoxy, cycloalkoxy, haloalkyl, cycloalkyl, heterocyclyl, aryl, or 5-10 membered heteroaryl is each optionally substituted with one to four Z¹ and wherein any two Z₁ can be combined with the atoms to which they are attached to form a C₃₋₁₀ cycloalkyl or heterocyclyl or C₆₋₁₀ aryl or 5-10 membered heteroaryl;
X is each independently —N═, —C(H)═, or —C(R⁸)═;
each R³ and R^{3′} is independently H, C₁₋₆ alkyl, C₁₋₆ haloalkyl, C₂₋₆ alkenyl, C₂₋₆ alkynyl, halogen, C₃₋₁₀ cycloalkyl, heterocyclyl, C₆₋₁₀ aryl, 5-10 membered heteroaryl, —CN, —NO₂, —OR^{3a}, —C(O)R^{3a}, —CH₂C(O)OR^{3a}, —C(O)OR^{3a}, —C(O)N(R^{3a})(R^{3b}), —N(R^{3a})C(O)R^{3b}, —N(R^{3a})C(O)OR^{3b}, —N(R^{3a})C(O)N(R^{3b})₂, —C(O)NHS(O)₂R^{3a}, —C(O)NR^{3a}S(O)₂R^{3b}, —C(O)NR^{3a}S(O)₂NR^{3b}R^{3c}, —C(O)NR^{3a}—S(O)(═NR^{3b})R^{3c}, —S(O)₂R^{3a}, —S(O)₂OR^{3a}, —S(O)₂N(R^{3a})(R^{3b}), —N(R^{3a})S(O)₂R^{3b}, —S(O)₂NHC(O)R^{3a}, —S(O)(═NR^{3a})R^{3b}, —S(O)(═NR^{3a})NR^{3b}, —S(═NR^{3a})(═NR^{3b})R^{3c}, —P(O)(OR^{3a})(R^{3b}), —P(O)(OR^{3a})(OR^{3b}), —B(OR^{3a})(OR^{3b}), or —O—C₁₋₆alkyl-C(O)OR^{3a}, wherein the alkyl, haloalkyl, alkenyl, alkynyl, cycloalkyl, heterocyclyl, aryl, or 5-10 membered heteroaryl in each instance is optionally substituted with one to four R^{3d};
each R^{3a}, R^{3b}, and R^{3c} is independently H, C₁₋₆ alkyl, C₆₋₁₀ aryl, 5-10 membered heteroaryl, C₁₋₆ haloalkyl, C₂₋₈ alkoxyalkyl, —C₁₋₄ alkyl-N(R^{9a})(R^{9b}), —C₁₋₄ alkyl-N(R^{9a})C(O)—O—C₁₋₄ alkyl-OP(O)(OR^{9c})₂, —C₁₋₄ alkyl-C(O)N(R^{9a})(R^{9b}), —C₁₋₄ alkyl-O—C(O)—O—C₁₋₄ alkyl, —C₁₋₄ alkyl-O—C(O)—C₁₋₄ alkyl-N(R^{9a})(R^{9b}), —C₁₋₄ alkyl-O—C(O)—C₁₋₄ alkyl-OP(O)(OR^{9c})₂, —C₁₋₄ alkyl-C₃₋₈ cycloalkyl, —C₁₋₄ alkyl-heterocyclyl, C₂₋₆ alkenyl, C₂₋₆ alkynyl, C₃₋₁₀ cycloalkyl, heterocyclyl, —CH₂CH(N(R^{9a})₂)C(O)OR^{9b}, C₁₋₆ alkyl-heterocyclyl, C₁₋₆ alkyl-aryl or C₁₋₆ alkyl-(5-10 membered) heteroaryl, wherein the alkyl, alkenyl, cycloalkyl, heterocyclyl, aryl, or 5-10 membered heteroaryl in each instance is optionally substituted with one to four Z^{1b};
each R^{5a} is independently H, C₁₋₆ alkyl, C₁₋₆ alkyloxy, C₁₋₆ haloalkyl, C₂₋₆ alkoxyalkyl, halogen, C₃₋₁₀ cycloalkyl, heterocyclyl, —C₁₋₆ alkyl-N(R^{9a})(R^{9b}), —CN, —OR^{5a1}, or —N(R^{5a1})(R^{5a2});
each R^{5a1} and R^{5a2} is independently H, C₁₋₆ alkyl, C₂₋₆ alkenyl, C₂₋₆ alkynyl, C₃₋₁₀ cycloalkyl, heterocyclyl, C₆₋₁₀ aryl, or 5-10 membered heteroaryl, wherein the cycloalkyl, heterocyclyl, aryl, or 5-10 membered heteroaryl in each instance is optionally substituted with one to four halogen;
each R^{3d} is independently H, C₁₋₆ alkyl, C₁₋₆ haloalkyl, C₁₋₆ alkoxy, C₁₋₆ haloalkoxy, C₂₋₆ alkoxyalkyl, C₂₋₆ alkenyl, C₂₋₆ alkynyl, halogen, C₃₋₁₀ cycloalkyl, heterocyclyl, C₆₋₁₀ aryl, 5-10 membered heteroaryl, oxo, —OH, —CN, CO₂R^{3e}, —NO₂, or —C(O)N(R^{2a})(R^{2b}), wherein the heterocyclyl or 5-10 membered heteroaryl is optionally substituted with C₁₋₆ alkyl, C₁₋₆ haloalkyl, or C₁₋₆ haloalkoxy;
each R⁸ is independently C₁₋₉ alkyl, C₁₋₈ haloalkyl, C₂₋₆ alkenyl, C₂₋₆ alkynyl, halogen, oxo, —OH, —CN, CO₂R^{3e}, —NO₂, —NH₂, —N₃, —SH, —O(C₁₋₉ alkyl), —O(C₁₋₈ haloalkyl), —NH(C₁₋₉ alkyl), —NH(C₁₋₈ haloalkyl), —N(C₁₋₉ alkyl)₂, —N(C₁₋₈ haloalkyl)₂;
each R^{9a} and R^{9b} is independently H, C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —NO₂, —CN, —N₃, —O—R^{12a}, —C(O)R^{12a}, —C(O)O—R^{12a}, —C(O)N(R^{12a})(R^{12b}), —N(R^{12a})(R^{12b}), —N(R^{12a})₂(R^{12b})⁺, —N(R^{12a})—C(O)R^{12b}, —N(R^{12a})C(O)O(R^{12b}), —N(R^{12a})C(O)N(R^{12b})(R^{12c}), —N(R^{12a})S(O)₂(R^{12b}), —N(R^{12a})S(O)₂—N(R^{12b})(R^{12c}), —N(R^{12a})S(O)₂O(R^{12b}), —OC(O)R^{12a}, —OC(O)OR^{12a}, —OC(O)—N(R^{12a})(R^{12b}), —S—R^{12a}, —S(O)R^{12a}, —S(O)(NH)R^{12a}, —S(O)₂R^{12a}, —S(O)₂N(R^{12a})(R^{12b}), —S(O)(NR^{12a})R^{12b}, or —Si(R^{12a})₃; or R^{9a} and R^{9b} together form a 3-10 membered heterocyclyl optionally substituted with C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, halogen, C₃₋₁₅ cycloalkyl, heterocyclyl, C₆₋₁₀ aryl, heteroaryl, oxo, —NO₂, —CN, —N₃, —O—R^{12a}, —C(O)R^{12a}, —C(O)O—R^{12a}, —C(O)N(R^{12a})(R^{12b}), —N(R^{12a})(R^{12b}), —N(R^{12a})₂(R^{12b})⁺, —N(R^{12a})—C(O)R^{12b}, —N(R^{12a})C(O)O(R^{12b}), —N(R^{12a})C(O)N(R^{12b})(R^{12c}), —N(R^{12a})S(O)₂(R^{12b}), —N(R^{12a})S(O)₂—N(R^{12b})(R^{12c}), —N(R^{12a})S(O)₂O(R^{12b}), —OC(O)R^{12a}, —OC(O)OR^{12a}, —OC(O)—N(R^{12a})(R^{12b}), —S—R^{12a}, —S(O)R^{12a}, —S(O)(NH)R^{12a}, —S(O)₂R^{12a}, —S(O)₂N(R^{12a})(R^{12b}), —S(O)(NR^{12a})R^{12b}, or —Si(R^{12a})₃;
each Z¹ is independently C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —NO₂, —N₃, —CN, or —O—R^{12a}, wherein the alkyl, haloalkyl, alkenyl, alkynyl, cycloalkyl, heterocyclyl, aryl, or 5-10 membered heteroaryl in each instance is optionally substituted with one to four Z^{1a};
each Z^{1a} is independently H, C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —NO₂, —CN, —N₃, —O—R^{12a}, —C(O)R^{12a}, —C(O)O—R^{12a}, —C(O)N(R^{12a})(R^{12b}), —N(R^{12a})(R^{12b}), —N(R^{12a})₂(R^{12b})⁺, —N(R^{12a})—C(O)R^{12b}, —N(R^{12a})C(O)O(R^{12b}), —N(R^{12a})C(O)N(R^{12b})(R^{12c}), —N(R^{12a})S(O)₂(R^{12b}), —N(R^{12a})S(O)₂—N(R^{12b})(R^{12c}), —N(R^{12a})S(O)₂O(R^{12b}), —OC(O)R^{12a}, —OC(O)OR^{12a}, —OC(O)—N(R^{12a})(R^{12b}), —S—R^{12a}, —S(O)R^{12a}, —S(O)(NH)R^{12a}, —S(O)₂R^{12a}, —S(O)₂N(R^{12a})(R^{12b}), —S(O)(NR^{12a})R^{12b}, or —Si(R^{12a})₃, wherein the alkyl, haloalkyl, alkenyl, alkynyl, cycloalkyl, heterocyclyl, aryl, or 5-10 membered heteroaryl in each instance is optionally substituted with one to four Z^{1b};
each Z^{1b} is independently H, C₁₋₉ alkyl, C₁₋₈ haloalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —OH, —CN, CO₂R^{3e}, —NO₂, —NH₂, —N₃, —SH, —O(C₁₋₉ alkyl), —O(C₁₋₈ haloalkyl), —O(C₂₋₆ alkenyl), —O(C₂₋₆ alkynyl), —O(C₃₋₁₅ cycloalkyl), —O(heterocyclyl), —O(C₆₋₁₀ aryl), —O(5-10 membered heteroaryl), —NH(C₁₋₉ alkyl), —NH(C₁₋₈ haloalkyl), —NH(C₂₋₆ alkenyl), —NH(C₂₋₆ alkynyl), —NH(C₃₋₁₅ cycloalkyl), —NH(heterocyclyl), —NH(C₆₋₁₀ aryl), —NH(5-10 membered heteroaryl), —N(cyclo-alkyl), —N(C₁₋₉ alkyl)₂, —N(C₁₋₈ haloalkyl)₂, —N(C₂₋₆ alkenyl)₂, —N(C₂₋₆ alkynyl)₂, —N(C₃₋₁₅ cycloalkyl)₂, —N(heterocyclyl)₂, —N(C₆₋₁₀ aryl)₂, —N(5-10 membered heteroaryl)₂, —N(C₁₋₉ alkyl)(C₁₋₈ haloalkyl), —N(C₁₋₉ alkyl)(C₂₋₆ alkenyl), —N(C₁₋₉ alkyl)(C₂₋₆ alkynyl), —N(C₁₋₉ alkyl)(C₃₋₁₅ cycloalkyl), —N(C₁₋₉ alkyl)(heterocyclyl), —N(C₁₋₉ alkyl)(C₆₋₁₀ aryl), —N(C₁₋₉ alkyl)(5-10 membered heteroaryl), —C(O)(C₁₋₉ alkyl), —C(O)(C₁₋₈ haloalkyl), —C(O)(C₂₋₆ alkenyl), —C(O)(C₂₋₆ alkynyl), —C(O)(C₃₋₁₅ cycloalkyl), —C(O)(heterocyclyl), —C(O)(C₆₋₁₀ aryl), —C(O)(5-10 membered heteroaryl), —C(O)O(C₁₋₉ alkyl), —C(O)O(C₁₋₈ haloalkyl), —C(O)O(C₂₋₆ alkenyl), —C(O)O(C₂₋₆ alkynyl), —C(O)O(C₃₋₁₅ cycloalkyl), —C(O)O(heterocyclyl), —C(O)O(C₆₋₁₀ aryl), —C(O)O(5-10 membered heteroaryl), —C(O)NH₂, —C(O)NH(C₁₋₉ alkyl), —C(O)NH(C₁₋₈ haloalkyl), —C(O)NH(C₂₋₆ alkenyl), —C(O)NH(C₂₋₆ alkynyl), —C(O)NH(C₃₋₁₅ cycloalkyl), —C(O)NH(heterocyclyl), —C(O)NH(C₆₋₁₀ aryl), —C(O)NH(5-10 membered heteroaryl), —C(O)N(C₁₋₉ alkyl)₂, —C(O)N(C₁₋₈ haloalkyl)₂, —C(O)N(C₂₋₆ alkenyl)₂, —C(O)N(C₂₋₆ alkynyl)₂, —C(O)N(C₃₋₁₅ cycloalkyl)₂, —C(O)N(heterocyclyl)₂, —C(O)N(C₆₋₁₀ aryl)₂, —C(O)N(5-10 membered heteroaryl)₂, —NHC(O)(C₁₋₉ alkyl), —NHC(O)(C₁₋₈ haloalkyl), —NHC(O)(C₂₋₆ alkenyl), —NHC(O)(C₂₋₆ alkynyl), —NHC(O)(C₃₋₁₅ cycloalkyl), —NHC(O)(heterocyclyl), —NHC(O)(C₆₋₁₀ aryl), —NHC(O)(5-10 membered heteroaryl), —NHC(O)O(C₁₋₉ alkyl), —NHC(O)O(C₁₋₈ haloalkyl), —NHC(O)O(C₂₋₆ alkenyl), —NHC(O)O(C₂₋₆ alkynyl), —NHC(O)O(C₃₋₁₅ cycloalkyl), —NHC(O)O(heterocyclyl), —NHC(O)O(C₆₋₁₀ aryl), —NHC(O)O(5-10 membered heteroaryl), —NHC(O)NH(C₁₋₉ alkyl), —NHC(O)NH(C₁₋₈ haloalkyl), —NHC(O)NH(C₂₋₆ alkenyl), —NHC(O)NH(C₂₋₆ alkynyl), —NHC(O)NH(C₃₋₁₅ cycloalkyl), —NHC(O)NH(heterocyclyl), —NHC(O)NH(C₆₋₁₀ aryl), or —NHC(O)NH(5-10 membered heteroaryl), wherein the alkyl, cycloalkyl, heterocyclyl, aryl, or 5-10 membered heteroaryl in each instance is optionally substituted with one to three C₁₋₉ alkyl, C₁₋₈ haloalkyl, halogen, —OH, —NH₂, CO₂H, —O(C₁₋₉ alkyl), —O(C₁₋₈ haloalkyl), —O(C₃₋₁₅ cycloalkyl), —O(heterocyclyl), —O(aryl), —O(5-10 membered heteroaryl), —NH(C₁₋₉ alkyl), —NH(C₁₋₈ haloalkyl), —NH(C₃₋₁₅ cycloalkyl), —NH(heterocyclyl), —NH(aryl), —NH(5-10 membered heteroaryl), —N(C₁₋₉ alkyl)₂, —N(C₃₋₁₅ cycloalkyl)₂, —NHC(O)(C₁₋₈ haloalkyl), —NHC(O)(C₃₋₁₅ cycloalkyl), —NHC(O)(heterocyclyl), —NHC(O)(aryl), —NHC(O)(5-10 membered heteroaryl), —NHC(O)O(C₁₋₉ alkyl), —NHC(O)O(C₁₋₈ haloalkyl), —NHC(O)O(C₂₋₆ alkynyl), —NHC(O)O(C₃₋₁₅ cycloalkyl), —NHC(O)O(heterocyclyl), —NHC(O)O(aryl), —NHC(O)O(5-10 membered heteroaryl), or —NHC(O)NH(C₁₋₉ alkyl);
each R^{1b}, R^{1c}, R^{2a}, R^{2b}, R^{4c}, R^{9c}, R^{12a}, R^{12b}, and R^{12c} is independently H, —OH, C₁₋₉ alkyl, C₂₋₆ alkenyl, C₂₋₆ alkynyl, C₃₋₁₅ cycloalkyl, heterocyclyl, C₆₋₁₀ aryl, or 5-10 membered heteroaryl, wherein the alkyl, alkenyl, alkynyl, cycloalkyl, heterocyclyl, aryl, or 5-10 membered heteroaryl in each instance is optionally substituted with one to four Z^{1b}; and
each R^{3e} is independently H, C₁₋₆ alkyl, C₁₋₆ haloalkyl, —C₁₋₄ alkyl-N(R^{9a})(R^{9b}), —C₁₋₄ alkyl-C(O)N(R^{9a})(R^{9b}), —C₁₋₄ alkyl-O—C(O)—O—C₁₋₄ alkyl, —C₁₋₄ alkyl-O—C(O)—C₁₋₄ alkyl-N(R^{9a})(R^{9b}), —C₁₋₄ alkyl-C₃₋₈ cycloalkyl, —C₁₋₄ alkyl-heterocyclyl, C₃₋₁₀ cycloalkyl, heterocyclyl, C₆₋₁₀ aryl, or 5-10 membered heteroaryl, wherein the alkyl, alkenyl, cycloalkyl, heterocyclyl, aryl, or 5-10 membered heteroaryl in each instance is optionally substituted with one to four Z^{1b}; wherein each 5-10 membered heteroaryl has 5 to 12 ring members and has one to four heteroatoms, each is independently N, O, or S; and wherein each heterocyclyl has 3 to 12 ring members and has one to four heteroatoms, each is independently N, O, or S.

4. The compound of claim 1, wherein the compound is a compound of Formula I-A-6 or Formula I-A-7 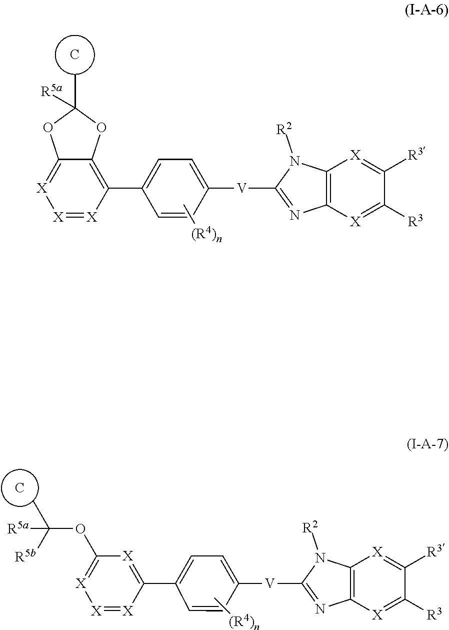
or a pharmaceutically acceptable salt thereof, wherein
Ring C is C₃₋₁₀ cycloalkyl, heterocyclyl, C₆₋₁₀ aryl or 5-10 membered heteroaryl, which is each optionally substituted with one to four Z¹;
V is CH₂, CH(CH₃), NH, C(O), S(O), S(O)₂;
n=0, 1, 2, 3, or 4;
R² is H, C₁₋₆ alkyl, —C₁₋₆ alkyl-alkoxy, —C₁₋₆ alkyl-cycloalkoxy, C₁₋₆ haloalkyl, C₂₋₆ alkenyl, C₂₋₆ alkynyl, C₃₋₁₀ cycloalkyl, heterocyclyl, C₆₋₁₀ aryl, 5-10 membered heteroaryl, —CN, —OR^{2a}, —S—R^{2a}, —S(O)R^{2a}, —S(O)(NH)R^{2a}, —S(O)₂R^{2a}, —S(O)₂N(R^{2a})(R^{2b}), or —S(O)(NR^{2a})R^{2b}, wherein the alkyl, alkoxy, cycloalkoxy, haloalkyl, cycloalkyl, heterocyclyl, aryl, or 5-10 membered heteroaryl in each instance is optionally substituted with one to four Z¹ and wherein any two Z₁ can be combined with the atoms to which they are attached to form a C₃₋₁₀ cycloalkyl or heterocyclyl or C₆₋₁₀ aryl or 5-10 membered heteroaryl;
X is each independently —N═, —C(H)═, or —C(R⁸)═;
each R³ and R^{3′} is independently H, C₁₋₆ alkyl, C₁₋₆ haloalkyl, C₂₋₆ alkenyl, C₂₋₆ alkynyl, halogen, C₃₋₁₀ cycloalkyl, heterocyclyl, C₆₋₁₀ aryl, 5-10 membered heteroaryl, —CN, —NO₂, —OR^{3a}, —C(O)R^{3a}, —CH₂C(O)OR^{3a}, —C(O)OR^{3a}, —C(O)N(R^{3a})(R^{3b}), —N(R^{3a})C(O)R^{3b}, —N(R^{3a})C(O)OR^{3b}, —N(R^{3a})C(O)N(R^{3b})₂, —C(O)NHS(O)₂R^{3a}, —C(O)NR^{3a}S(O)₂R^{3b}, —C(O)NR^{3a}S(O)₂NR^{3b}R^{3c}, —C(O)NR^{3a}—S(O)(═NR^{3b})R^{3c}, —S(O)₂R^{3a}, —S(O)₂OR^{3a}, —S(O)₂N(R^{3a})(R^{3b}), —N(R^{3a})S(O)₂R^{3b}, —S(O)₂NHC(O)R^{3a}, —S(O)(═NR^{3a})R^{3b}, —S(O)(═NR^{3a})NR^{3b}, —S(═NR^{3a})(═NR^{3b})R^{3c}, —P(O)(OR^{3a})(R^{3b}), —P(O)(OR^{3a})(OR^{3b}), —B(OR^{3a})(OR^{3b}), or —O—C₁₋₆alkyl-C(O)OR^{3a}, wherein the alkyl, haloalkyl, alkenyl, alkynyl, cycloalkyl, heterocyclyl, aryl, or 5-10 membered heteroaryl in each instance is optionally substituted with one to four R^{3d};
each R^{3a}, R^{3b}, and R^{3c} is independently H, C₁₋₆ alkyl, C₆₋₁₀ aryl, 5-10 membered heteroaryl, C₁₋₆ haloalkyl, C₂₋₈ alkoxyalkyl, —C₁₋₄ alkyl-N(R^{9a})(R^{9b}), —C₁₋₄ alkyl-N(R^{9a})C(O)—O—C₁₋₄ alkyl-OP(O)(OR^{9c})₂, —C₁₋₄ alkyl-C(O)N(R^{9a})(R^{9b}), —C1-4 alkyl-O—C(O)—O—C1-4 alkyl, —C₁₋₄ alkyl-O—C(O)—C₁₋₄ alkyl-N(R^{9a})(R^{9b}), —C₁₋₄ alkyl-O—C(O)—C₁₋₄ alkyl-OP(O)(OR^{9c})₂, —C₁₋₄ alkyl-C₃₋₈ cycloalkyl, —C₁₋₄ alkyl-heterocyclyl, C₂₋₆ alkenyl, C₂₋₆ alkynyl, C₃₋₁₀ cycloalkyl, heterocyclyl, —CH₂CH(N(R^{9a})₂)C(O)OR^{9b}, C₁₋₆ alkyl-heterocyclyl, C₁₋₆ alkyl-aryl or C₁₋₆ alkyl-(5-10 membered) heteroaryl, wherein the alkyl, alkenyl, cycloalkyl, heterocyclyl, aryl, or 5-10 membered heteroaryl in each instance is optionally substituted with one to four Z^{1b};
each R⁴ is independently H, CH₂OH, —OH, C₁₋₉ alkyl, C₁₋₈ haloalkyl, C₁₋₆ haloalkoxy, C₂₋₆ alkoxyalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —NO₂, —CN, —N₃, —O—R^{4a}, —C(O)R^{4a}, —C(O)O—R^{4a}, —C(O)N(R^{4a})(R^{4b}), —N(R^{4a})(R^{4b}), —N(R^{4a})₂(R^{4b})⁺, —N(R^{4a})—C(O)R^{4b}, —N(R^{4a})C(O)O(R^{4b}), —N(R^{4a})C(O)N(R^{4b})(R^{4c}), —N(R^{4a})S(O)₂(R^{4b}), —N(R^{4a})S(O)₂—N(R^{4b})(R^{4c}), —N(R^{4a})S(O)₂O(R^{4b}), —OC(O)R^{4a}, —OC(O)OR^{4a}, —OC(O)—N(R^{4a})(R^{4b}), —S—R^{4a}, —S(O)R^{4a}, —S(O)(NH)R^{4a}, —S(O)₂R^{4a}, —S(O)₂N(R^{4a})(R^{4b}), —S(O)(NR^{4a})R^{4b}, or —Si(R^{4a})₃, wherein the alkyl, haloalkyl, alkenyl, alkynyl, cycloalkyl, heterocyclyl, aryl, or 5-10 membered heteroaryl in each instance is optionally substituted with one to four Z^{1b}; or two R⁴ groups attached to adjacent ring atoms are combined with the atoms to which they are attached to form a C₅₋₁₀ cycloalkyl or heterocyclyl, which is each optionally substituted with one to four Z^{1b};
each R^{5a} and R^{5b} is independently H, C₁₋₆ alkyl, C₁₋₆ alkyloxy, C₁₋₆ haloalkyl, C₂₋₆ alkoxyalkyl, halogen, C₃₋₁₀ cycloalkyl, heterocyclyl, —C₁₋₆ alkyl-N(R^{9a})(R^{9b}), —CN, —OR^{5a1}, or —N(R^{5a1})(R^{5a2}); or R^{5a} and R^{5b} are combined with the atoms to which they are attached to form a C₃₋₁₀ cycloalkyl or heterocyclyl, which is each optionally substituted with one to four R^{5a3};
each R^{5a1} and R^{5a2} is independently H, C₁₋₆ alkyl, C₂₋₆ alkenyl, C₂₋₆ alkynyl, C₃₋₁₀ cycloalkyl, heterocyclyl, C₆₋₁₀ aryl, or 5-10 membered heteroaryl, wherein the cycloalkyl, heterocyclyl, aryl, or 5-10 membered heteroaryl in each instance is optionally substituted with one to four halogen;
each R^{3d} and R^{5a3} is independently H, C₁₋₆ alkyl, C₁₋₆ haloalkyl, C₁₋₆ alkoxy, C₁₋₆ haloalkoxy, C₂₋₆ alkoxyalkyl, C₂₋₆ alkenyl, C₂₋₆ alkynyl, halogen, C₃₋₁₀ cycloalkyl, heterocyclyl, C₆₋₁₀ aryl, 5-10 membered heteroaryl, oxo, —OH, —CN, CO₂R^{3e}, —NO₂, or —C(O)N(R^{2a})(R^{2b}), wherein the heterocyclyl or 5-10 membered heteroaryl in each instance is optionally substituted with C₁₋₆ alkyl, C₁₋₆ haloalkyl, or C₁₋₆ haloalkoxy;
each R⁸ is independently C₁₋₉ alkyl, C₁₋₈ haloalkyl, C₂₋₆ alkenyl, C₂₋₆ alkynyl, halogen, oxo, —OH, —CN, CO₂R^{3e}, —NO₂, —NH₂, —N₃, —SH, —O(C₁₋₉ alkyl), —O(C₁₋₈ haloalkyl), —NH(C₁₋₉ alkyl), —NH(C₁₋₈ haloalkyl), —N(C₁₋₉ alkyl)₂, —N(C₁₋₈ haloalkyl)₂;
each R^{9a} and R^{9b} is independently H, C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —NO₂, —CN, —N₃, —O—R^{12a}, —C(O)R^{12a}, —C(O)O—R^{12a}, —C(O)N(R^{12a})(R^{12b}), —N(R^{12a})(R^{12b}), —N(R^{12a})₂(R^{12b})⁺, —N(R^{12a})—C(O)R^{12b}, —N(R^{12a})C(O)O(R^{12b}), —N(R^{12a})C(O)N(R^{12b})(R^{12c}), —N(R^{12a})S(O)₂(R^{12b}), —N(R^{12a})S(O)₂—N(R^{12b})(R^{12c}), —N(R^{12a})S(O)₂O(R^{12b}), —OC(O)R^{12a}, —OC(O)OR^{12a}, —OC(O)—N(R^{12a})(R^{12b}), —S—R^{12a}, —S(O)R^{12a}, —S(O)(NH)R^{12a}, —S(O)₂R^{12a}, —S(O)₂N(R^{12a})(R^{12b}), —S(O)(NR^{12a})R^{12b}, or —Si(R^{12a})₃; or R^{9a} and R^{9b} together form a 3-10 membered heterocyclyl optionally substituted with C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —NO₂, —CN, —N₃, —O—R^{12a}, —C(O)R^{12a}, —C(O)O—R^{12a}, —C(O)N(R^{12a})(R^{12b}), —N(R^{12a})(R^{12b}), —N(R^{12a})₂(R^{12b})⁺, —N(R^{12a})—C(O)R^{12b}, —N(R^{12a})C(O)O(R^{12b}), —N(R^{12a})C(O)N(R^{12b})(R^{12c}), —N(R^{12a})S(O)₂(R^{12b}), —N(R^{12a})S(O)₂—N(R^{12b})(R^{12c}), —N(R^{12a})S(O)₂O(R^{12b}), —OC(O)R^{12a}, —OC(O)OR^{12a}, —OC(O)—N(R^{12a})(R^{12b}), —S—R^{12a}, —S(O)R^{12a}, —S(O)(NH)R^{12a}, —S(O)₂R^{12a}, —S(O)₂N(R^{12a})(R^{12b}), —S(O)(NR^{12a})R^{12b}, or —Si(R^{12a})₃;
each Z¹ is independently C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —NO₂, —N₃, —CN, or —O—R^{12a}, wherein the alkyl, haloalkyl, alkenyl, alkynyl, cycloalkyl, heterocyclyl, aryl, or 5-10 membered heteroaryl in each instance is optionally substituted with one to four Z^{1a};
each Z^{1a} is independently H, C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —NO₂, —CN, —N₃, —O—R^{12a}, —C(O)R^{12a}, —C(O)O—R^{12a}, —C(O)N(R^{12a})(R^{12b}), —N(R^{12a})(R^{12b}), —N(R^{12a})₂(R^{12b})⁺, —N(R^{12a})—C(O)R^{12b}, —N(R^{12a})C(O)O(R^{12b}), —N(R^{12a})C(O)N(R^{12b})(R^{12c}), —N(R^{12a})S(O)₂(R^{12b}), —N(R^{12a})S(O)₂—N(R^{12b})(R^{12c}), —N(R^{12a})S(O)₂O(R^{12b}), —OC(O)R^{12a}, —OC(O)OR^{12a}, —OC(O)—N(R^{12a})(R^{12b}), —S—R^{12a}, —S(O)R^{12a}, —S(O)(NH)R^{12a}, —S(O)₂R^{12a}, —S(O)₂N(R^{12a})(R^{12b}), —S(O)(NR^{12a})R^{12b}, or —Si(R^{12a})₃, wherein the alkyl, haloalkyl, alkenyl, alkynyl, cycloalkyl, heterocyclyl, aryl, or 5-10 membered heteroaryl in each instance is optionally substituted with one to four Z^{1b};
each Z^{1b} is independently H, C₁₋₉ alkyl, C₁₋₈ haloalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —OH, —CN, CO₂R^{3e}, —NO₂, —NH₂, —N₃, —SH, —O(C₁₋₉ alkyl), —O(C₁₋₈ haloalkyl), —O(C₂₋₆ alkenyl), —O(C₂₋₆ alkynyl), —O(C₃₋₁₅ cycloalkyl), —O(heterocyclyl), —O(C₆₋₁₀ aryl), —O(5-10 membered heteroaryl), —NH(C₁₋₉ alkyl), —NH(C₁₋₈ haloalkyl), —NH(C₂₋₆ alkenyl), —NH(C₂₋₆ alkynyl), —NH(C₃₋₁₅ cycloalkyl), —NH(heterocyclyl), —NH(C₆₋₁₀ aryl), —NH(5-10 membered heteroaryl), —N(cyclo-alkyl), —N(C₁₋₉ alkyl)₂, —N(C₁₋₈ haloalkyl)₂, —N(C₂₋₆ alkenyl)₂, —N(C₂₋₆ alkynyl)₂, —N(C₃₋₁₅ cycloalkyl)₂, —N(heterocyclyl)₂, —N(C₆₋₁₀ aryl)₂, —N(5-10 membered heteroaryl)₂, —N(C₁₋₉ alkyl)(C₁₋₈ haloalkyl), —N(C₁₋₉ alkyl)(C₂₋₆ alkenyl), —N(C₁₋₉ alkyl)(C₂₋₆ alkynyl), —N(C₁₋₉ alkyl)(C₃₋₁₅ cycloalkyl), —N(C₁₋₉ alkyl)(heterocyclyl), —N(C₁₋₉ alkyl)(C₆₋₁₀ aryl), —N(C₁₋₉ alkyl)(5-10 membered heteroaryl), —C(O)(C₁₋₉ alkyl), —C(O)(C₁₋₈ haloalkyl), —C(O)(C₂₋₆ alkenyl), —C(O)(C₂₋₆ alkynyl), —C(O)(C₃₋₁₅ cycloalkyl), —C(O)(heterocyclyl), —C(O)(C₆₋₁₀ aryl), —C(O)(5-10 membered heteroaryl), —C(O)O(C₁₋₉ alkyl), —C(O)O(C₁₋₈ haloalkyl), —C(O)O(C₂₋₆ alkenyl), —C(O)O(C₂₋₆ alkynyl), —C(O)O(C₃₋₁₅ cycloalkyl), —C(O)O(heterocyclyl), —C(O)O(C₆₋₁₀ aryl), —C(O)O(5-10 membered heteroaryl), —C(O)NH₂, —C(O)NH(C₁₋₉ alkyl), —C(O)NH(C₁₋₈ haloalkyl), —C(O)NH(C₂₋₆ alkenyl), —C(O)NH(C₂₋₆ alkynyl), —C(O)NH(C₃₋₁₅ cycloalkyl), —C(O)NH(heterocyclyl), —C(O)NH(C₆₋₁₀ aryl), —C(O)NH(5-10 membered heteroaryl), —C(O)N(C₁₋₉ alkyl)₂, —C(O)N(C₁₋₈ haloalkyl)₂, —C(O)N(C₂₋₆ alkenyl)₂, —C(O)N(C₂₋₆ alkynyl)₂, —C(O)N(C₃₋₁₅ cycloalkyl)₂, —C(O)N(heterocyclyl)₂, —C(O)N(C₆₋₁₀ aryl)₂, —C(O)N(5-10 membered heteroaryl)₂, —NHC(O)(C₁₋₉ alkyl), —NHC(O)(C₁₋₈ haloalkyl), —NHC(O)(C₂₋₆ alkenyl), —NHC(O)(C₂₋₆ alkynyl), —NHC(O)(C₃₋₁₅ cycloalkyl), —NHC(O)(heterocyclyl), —NHC(O)(C₆₋₁₀ aryl), —NHC(O)(5-10 membered heteroaryl), —NHC(O)O(C₁₋₉ alkyl), —NHC(O)O(C₁₋₈ haloalkyl), —NHC(O)O(C₂₋₆ alkenyl), —NHC(O)O(C₂₋₆ alkynyl), —NHC(O)O(C₃₋₁₅ cycloalkyl), —NHC(O)O(heterocyclyl), —NHC(O)O(C₆₋₁₀ aryl), —NHC(O)O(5-10 membered heteroaryl), —NHC(O)NH(C₁₋₉ alkyl), —NHC(O)NH(C₁₋₈ haloalkyl), —NHC(O)NH(C₂₋₆ alkenyl), —NHC(O)NH(C₂₋₆ alkynyl), —NHC(O)NH(C₃₋₁₅ cycloalkyl), —NHC(O)NH(heterocyclyl), —NHC(O)NH(C₆₋₁₀ aryl), or —NHC(O)NH(5-10 membered heteroaryl), wherein the alkyl, cycloalkyl, heterocyclyl, aryl, or 5-10 membered heteroaryl in each instance is optionally substituted with one to three C₁₋₉ alkyl, C₁₋₈ haloalkyl, halogen, —OH, —NH₂, CO₂H, —O(C₁₋₉ alkyl), —O(C₁₋₈ haloalkyl), —O(C₃₋₁₅ cycloalkyl), —O(heterocyclyl), —O(aryl), —O(5-10 membered heteroaryl), —NH(C₁₋₉ alkyl), —NH(C₁₋₈ haloalkyl), —NH(C₃₋₁₅ cycloalkyl), —NH(heterocyclyl), —NH(aryl), —NH(5-10 membered heteroaryl), —N(C₁₋₉ alkyl)₂, —N(C₃₋₁₅ cycloalkyl)₂, —NHC(O)(C₁₋₈ haloalkyl), —NHC(O)(C₃₋₁₅ cycloalkyl), —NHC(O)(heterocyclyl), —NHC(O)(aryl), —NHC(O)(5-10 membered heteroaryl), —NHC(O)O(C₁₋₉ alkyl), —NHC(O)O(C₁₋₈ haloalkyl), —NHC(O)O(C₂₋₆ alkynyl), —NHC(O)O(C₃₋₁₅ cycloalkyl), —NHC(O)O(heterocyclyl), —NHC(O)O(aryl), —NHC(O)O(5-10 membered heteroaryl), or —NHC(O)NH(C₁₋₉ alkyl;
each R^{1b}, R^{1c}, R^{2a}, R^{2b}, R^{4a}, R^{4b}, R^{4c}, R^{9c}, R^{12a}, R^{12b}, and R^{12c} is independently H, —OH, C₁₋₉ alkyl, C₂₋₆ alkenyl, C₂₋₆ alkynyl, C₃₋₁₅ cycloalkyl, heterocyclyl, C₆₋₁₀ aryl, or 5-10 membered heteroaryl, wherein the alkyl, alkenyl, alkynyl, cycloalkyl, heterocyclyl, aryl, or 5-10 membered heteroaryl in each instance is optionally substituted with one to four Z^{1b}; and
each R^{3e} is independently H, C₁₋₆ alkyl, C₁₋₆ haloalkyl, —C₁₋₄ alkyl-N(R^{9a})(R^{9b}), —C₁₋₄ alkyl-C(O)N(R^{9a})(R^{9b}), —C₁₋₄ alkyl-O—C(O)—O—C₁₋₄ alkyl, —C₁₋₄ alkyl-O—C(O)—C₁₋₄ alkyl-N(R^{9a})(R^{9b}), —C₁₋₄ alkyl-C₃₋₈ cycloalkyl, —C₁₋₄ alkyl-heterocyclyl, C₃₋₁₀ cycloalkyl, heterocyclyl, C₆₋₁₀ aryl, or 5-10 membered heteroaryl, wherein the alkyl, alkenyl, cycloalkyl, heterocyclyl, aryl, or 5-10 membered heteroaryl in each instance is optionally substituted with one to four Z^{1b};
wherein each 5-10 membered heteroaryl has 5 to 12 ring members and has one to four heteroatoms, each is independently N, O, or S;
and wherein each heterocyclyl has 3 to 12 ring members and has one to four heteroatoms, each is independently N, O, or S.

5. The compound of claim 2, or a pharmaceutically acceptable salt thereof, wherein each R^{5a} and R^{5b} is independently H, C₁₋₉ alkyl, or C₂₋₆ alkoxyalkyl and wherein the C₁₋₉ alkyl or C₂₋₆ alkoxyalkyl in each instance is optionally substituted with one or more halogens.

6. The compound of claim 3, or a pharmaceutically acceptable salt thereof, wherein R^{5a} is independently H, C₁₋₉ alkyl, or C₂₋₆ alkoxyalkyl and wherein the C₁₋₉ alkyl or C₂₋₆ alkoxyalkyl in each instance is optionally substituted with one or more halogens.

7. The compound of claim 4, or a pharmaceutically acceptable salt thereof, wherein each R^{5a} and R^{5b} is independently H, C₁₋₉ alkyl, or C₂₋₆ alkoxyalkyl and wherein the C₁₋₉ alkyl or C₂₋₆ alkoxy in each instance is optionally substituted with one or more halogens.

8. The compound of claim 2, or a pharmaceutically acceptable salt thereof, wherein Ring C is C₆₋₁₀ aryl or 5-10 membered heteroaryl selected from the group consisting of 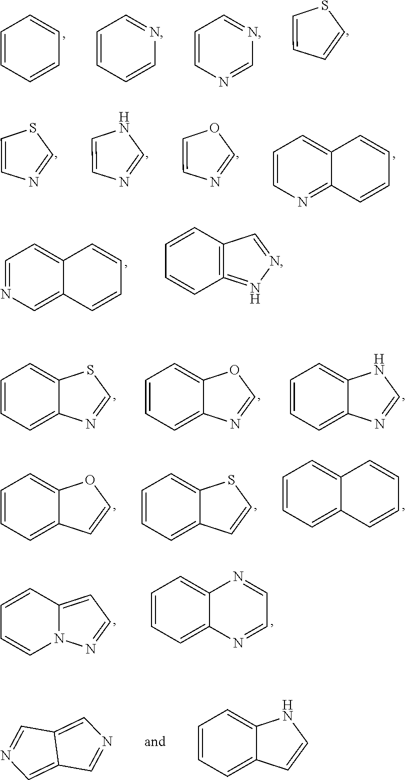
which is each optionally substituted with one to four substituents, each independently selected from the group consisting of H, OH, oxo, halogen, C₁₋₆ alkyl, C₁₋₆ haloalkyl, C₁₋₆ alkoxy, C₁₋₆ haloalkoxy, C₂₋₆ alkoxyalkyl, C₂₋₆ alkenyl, C₂₋₆ alkynyl, C₃₋₁₀ cycloalkyl, heterocyclyl, —NO₂, —N₃, —CN, —O—C₁₋₆ alkyl, —C(O)—C₁₋₆ alkyl, —C(O)—N(C₀₋₆ alkyl)(C₀₋₆ alkyl), and —N(C₀₋₆ alkyl)(C₀₋₆ alkyl),
wherein the one to four substituents in each instance is optionally substituted with one or more halogens.

9. The compound of claim 3, or a pharmaceutically acceptable salt thereof, wherein Ring C is C₆₋₁₀ aryl or 5-10 membered heteroaryl selected from the group consisting of 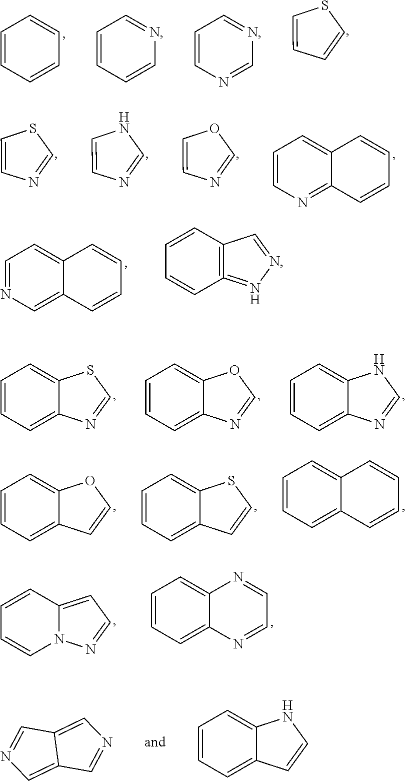
which is each optionally substituted with one to four substituents each independently selected from the group consisting of H, OH, oxo, halogen, C₁₋₆ alkyl, C₁₋₆ haloalkyl, C₁₋₆ alkoxy, C₁₋₆ haloalkoxy, C₂₋₆ alkoxyalkyl, C₂₋₆ alkenyl, C₂₋₆ alkynyl, C₃₋₁₀ cycloalkyl, heterocyclyl, —NO₂, —N₃, —CN, —O—C₁₋₆ alkyl, —C(O)—C₁₋₆ alkyl, —C(O)—N(C₀₋₆ alkyl)(C₀₋₆ alkyl), and —N(C₀₋₆ alkyl)(C₀₋₆ alkyl),
wherein the one to four substituents in each instance is optionally substituted with one or more halogens.

10. The compound of claim 4, or a pharmaceutically acceptable salt thereof, wherein Ring C is C₆₋₁₀ aryl or 5-10 membered heteroaryl selected from the group consisting of 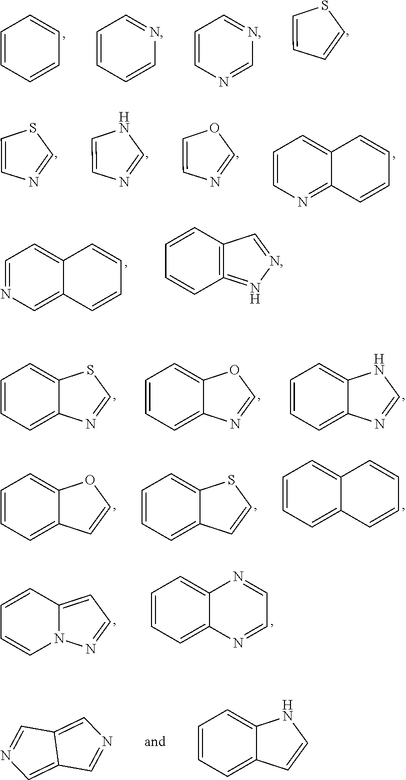
which is each optionally substituted with one to four substituents each independently selected from the group consisting of H, OH, oxo, halogen, C₁₋₆ alkyl, C₁₋₆ haloalkyl, C₁₋₆ alkoxy, C₁₋₆ haloalkoxy, C₂₋₆ alkoxyalkyl, C₂₋₆ alkenyl, C₂₋₆ alkynyl, C₃₋₁₀ cycloalkyl, heterocyclyl, —NO₂, —N₃, —CN, —O—C₁₋₆ alkyl, —C(O)—C₁₋₆ alkyl, —C(O)—N(C₀₋₆ alkyl)(C₀₋₆ alkyl), and —N(C₀₋₆ alkyl)(C₀₋₆ alkyl),
wherein the one to four substituents in each instance is optionally substituted with one or more halogens

11. The compound of claim 1, or a pharmaceutically acceptable salt thereof, wherein Ring A is selected from the group consisting of 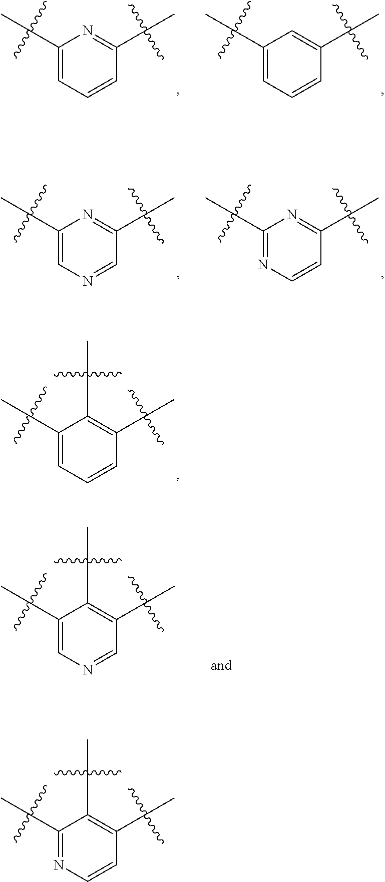
which is each optionally substituted with one to four Z^{1a}.

12. The compound of claim 11, wherein each of the one to four Z^{1a} is independently H, halogen, C₁₋₉ alkyl, C₁₋₆ alkoxy, C₂₋₆ alkoxyalkyl, C₂₋₆ alkenyl, C₂₋₆ alkynyl, C₃₋₁₅ cycloalkyl, or heterocyclyl, which is each optionally substituted with one or more halogens.

13. The compound of claim 1, or a pharmaceutically acceptable salt thereof, wherein R² is C₁₋₆ alkyl, C₁₋₆ alkyl-alkoxy, C₁₋₆ alkyl-cycloalkoxy, C₁₋₆ haloalkyl, C₂₋₆ alkenyl, C₂₋₆ alkynyl, C₃₋₁₀ cycloalkyl, heterocyclyl, C₆₋₁₀ aryl, or 5-10 membered heteroaryl, which is each optionally substituted with one to four Z¹.

14. The compound of claim 13, or a pharmaceutically acceptable salt thereof,
wherein R² is selected from the group consisting of 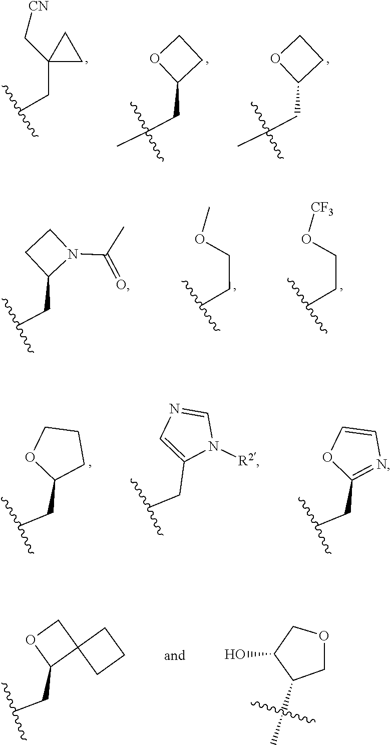
which is each optionally substituted with one to four Z¹,
wherein R^{2′} is H, C₁₋₆ alkyl, C₁₋₆ haloalkyl, or —C₁₋₆ alkyl-cycloalkoxy, which is each optionally substituted with one or more halogens.

15. The compound of claim 1, or a pharmaceutically acceptable salt thereof, wherein each R³ and R^{3′} is independently H, C₆₋₁₀ aryl, 5-10 membered heteroaryl, —C(O)R^{3a}, —CH₂C(O)OR^{3a}, —C(O)N(R^{3a})(R^{3b}), —N(R^{3a})C(O)R^{3b}, —N(R^{3a})C(O)OR^{3b}, —N(R^{3a})C(O)N(R^{3b})₂, —C(O)NHS(O)₂R^{3a}, —C(O)NR^{3a}S(O)₂R^{3b}, —C(O)NR^{3a}S(O)₂NR^{3b}R^{3c}, —C(O)NR^{3a}—S(O)(═NR^{3b})R^{3c}, —S(O)₂N(R^{3a})(R^{3b}), —N(R^{3a})S(O)₂R^{3b}, —S(O)₂NHC(O)R^{3a}, or —O—C₁₋₆alkyl-C(O)OR^{3a}, which is each optionally substituted with one to four R^{3d}.

16. The compound of claim 1, or a pharmaceutically acceptable salt thereof, wherein each R³ and R^{3′} is independently H, C(O)NR^{3a}(CH₂)ₘR^{3b}, C(O)NR^{3a}(CH₂)ₘC(O)R^{3b}, NR^{3a} C(O) (CH₂)ₘR^{3b}, NR^{3a} (CH₂)ₘ R^{3b}, NR^{3a (}CH₂)ₘ C(O)R^{3b}, C(O)NR^{3a}R^{3b}, or NR^{3a}C(O)R^{3b}, which is each optionally substituted with one to four R^{3d}, wherein m=0, 1, 2, or 3.

17. The compound of claim 16, or a pharmaceutically acceptable salt thereof, wherein each R³ and R^{3′} is independently H, C(O)NHR^{3b}, or NHC(O)R^{3b}.

18. The compound of claim 17, or a pharmaceutically acceptable salt thereof, wherein each R^{3b} is independently H, C₁₋₆ alkyl, C₁₋₆ haloalkyl, C₂₋₈ alkoxyalkyl, —C₁₋₄ alkyl-N(R^{9a})(R^{9b}), —C₁₋₄ alkyl-C(O)N(R^{9a})(R^{9b}), —C₁₋₄ alkyl-O—C(O)—O—C₁₋₄ alkyl, —C₁₋₄ alkyl-O—C(O)—C₁₋₄ alkyl-N(R^{9a})(R^{9b}), —C₁₋₄ alkyl-C₃₋₈ cycloalkyl, —C₁₋₄ alkyl-heterocyclyl, C₂₋₆ alkenyl, C₂₋₆ alkynyl, C₃₋₁₀ cycloalkyl, heterocyclyl, C₆₋₁₀ aryl, 5-10 membered heteroaryl, —CH₂CH(N(R^{9a})₂)C(O)OR^{9b}, C1-6 alkyl-heterocyclyl, C1-6 alkyl-aryl or C1-6 alkyl-(5-10 membered) heteroaryl, wherein the alkyl, alkenyl, cycloalkyl, heterocyclyl, aryl, or 5-10 membered heteroaryl in each instance is optionally substituted with one to four Z^{1b}.

19. The compound of claim 17, or a pharmaceutically acceptable salt thereof, wherein R^{3b} is C₆₋₁₀ aryl or 5-10 membered heteroaryl, which in each instance is optionally substituted with one to five R^{3f}, wherein R^{3f} is H, OH, oxo, NH₂, halogen, —NO₂, —N₃, —CN, C₁₋₆ alkyl, C₁₋₆ haloalkyl, C₁₋₆ alkoxy, C₁₋₆ haloalkoxy, C₂₋₆ alkoxyalkyl, C₂₋₆ alkenyl, C₂₋₆ alkynyl, C₃₋₁₀ cycloalkyl, heterocyclyl, —O—C₁₋₆ alkyl, —C(O)—C₁₋₆ alkyl, —C(O)—N(R^{12a})(R^{12b}), —N(R^{12a})(R^{12b}), C6-10 aryl, or 5-10 membered heteroaryl, wherein R^{3f} in each instance is optionally substituted with one or more halogen, C₁₋₆ alkyl, C₁₋₆ haloalkyl, C₁₋₆ alkoxy, or C₁₋₆ haloalkoxy.

20. The compound of claim 19, or a pharmaceutically acceptable salt thereof, wherein the aryl or 5-10 membered heteroaryl in R^{3b} is selected from the group consisting of: 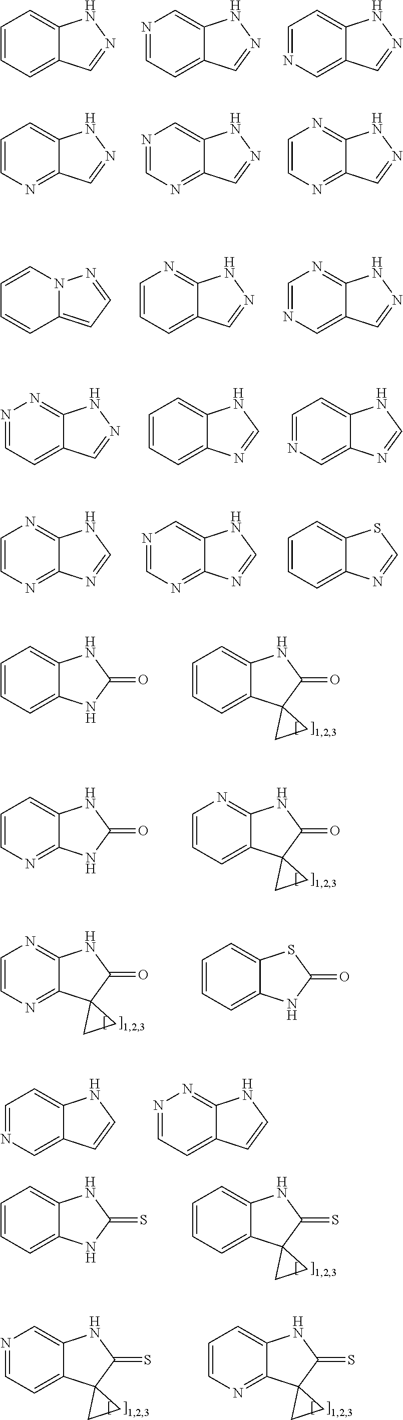 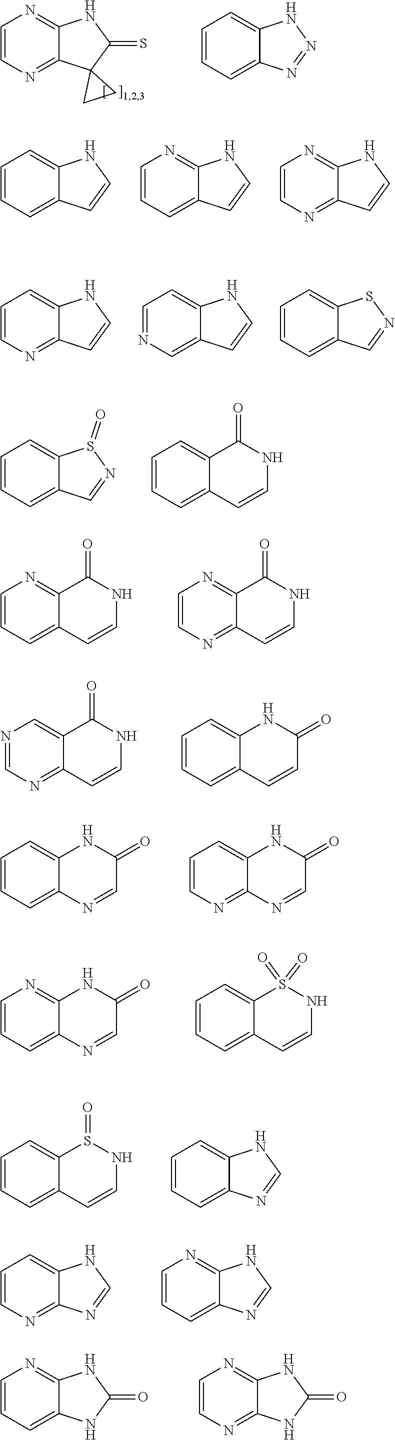 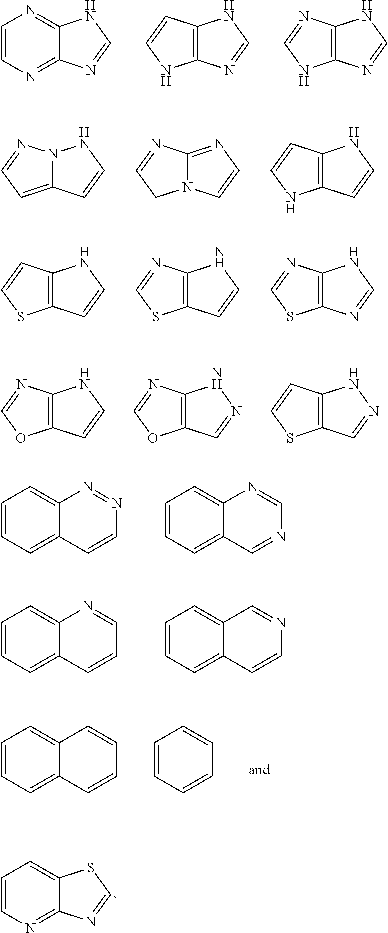
which is each optionally substituted with one to five R^{3f}.

21. The compound of claim 1, or a pharmaceutically acceptable salt thereof, wherein R⁴ is independently H, —OH, —CH₂OH, C₁₋₉ alkyl, C₁₋₈ haloalkyl, C₁₋₆ haloalkoxy, C₂₋₆ alkoxyalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —NO₂, —CN, —N₃, —O—C₁₋₉ alkyl, or —C(O) C₁₋₉ alkyl, which is each optionally substituted with one to four Z^{1b},
wherein two R⁴ groups attached to adjacent ring atoms may be combined with the atoms to which they are attached to form a C₅₋₁₀ cycloalkyl or heterocyclyl, which is each optionally substituted with one to four Z^{1b}.

22. The compound of claim 21, or a pharmaceutically acceptable salt thereof, wherein R⁴ is independently H, halogen, —CH₂OH, C₁₋₉ alkyl, C₁₋₈ haloalkyl, C₁₋₆ haloalkoxy, C₂₋₆ alkoxyalkyl, C₂₋₆ alkenyl, C₂₋₆ alkynyl, or C₃₋₁₅ cycloalkyl.

23. The compound of claim 1, or a pharmaceutically acceptable salt thereof, wherein Z¹ is independently C₁₋₉ alkyl, C₁₋₈ haloalkyl, C₁₋₆ alkoxy, C₁₋₆ haloalkoxy, C₂₋₆ alkoxyalkyl, C₂₋₆ alkenyl, C₂₋₆ alkynyl, halogen, C₃₋₁₅ cycloalkyl, heterocyclyl, C₆₋₁₀ aryl, 5-10 membered heteroaryl, oxo, —NO₂, —N₃, —CN, —O—R^{12a}, —C(O)—R^{12a}, —C(O)O—R^{12a}, —C(O)—N(R^{12a})(R^{12b}), or —N(R^{12a})(R^{12b}).

24. The compound of claim 1, or a pharmaceutically acceptable salt thereof, wherein Z^{1a} is independently H, —OH, C₁₋₉ alkyl, C₁₋₈ haloalkyl, C₁₋₆ alkoxy, C₁₋₆ haloalkoxy, C₂₋₆ alkoxyalkyl, or halogen.

25. The compound of claim 1, or a pharmaceutically acceptable salt thereof, wherein Z^{1b} is independently H, C₁₋₉ alkyl, C₁₋₈ haloalkyl, halogen, oxo, —OH, or —CN.

26. The compound of claim 1, or a pharmaceutically acceptable salt thereof, wherein R⁸ is independently H, oxo, OH, C₁₋₉ alkyl, C₁₋₈ haloalkyl, halogen, —O(C₁₋₃ alkyl), or —O(C₁₋₃ haloalkyl).

27. The compound of claim 1, or a pharmaceutically acceptable salt thereof, having a structure selected from the group consisting of

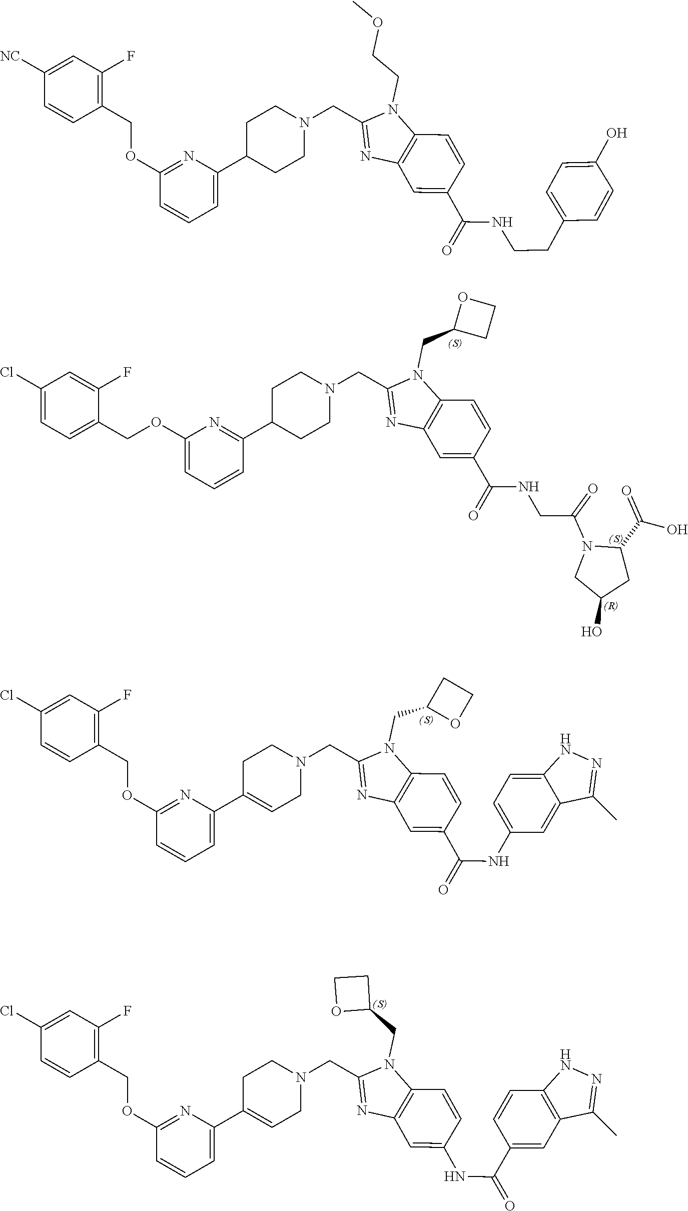

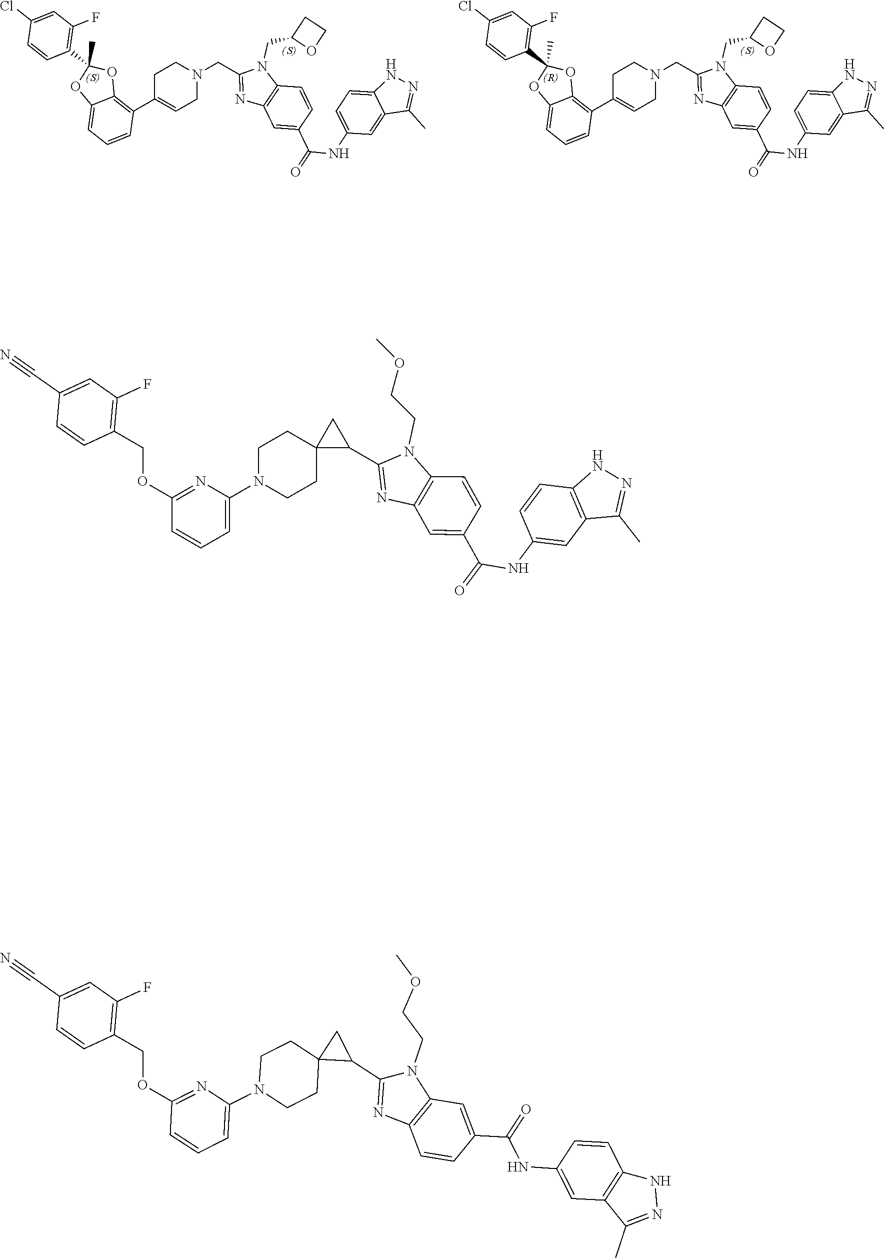

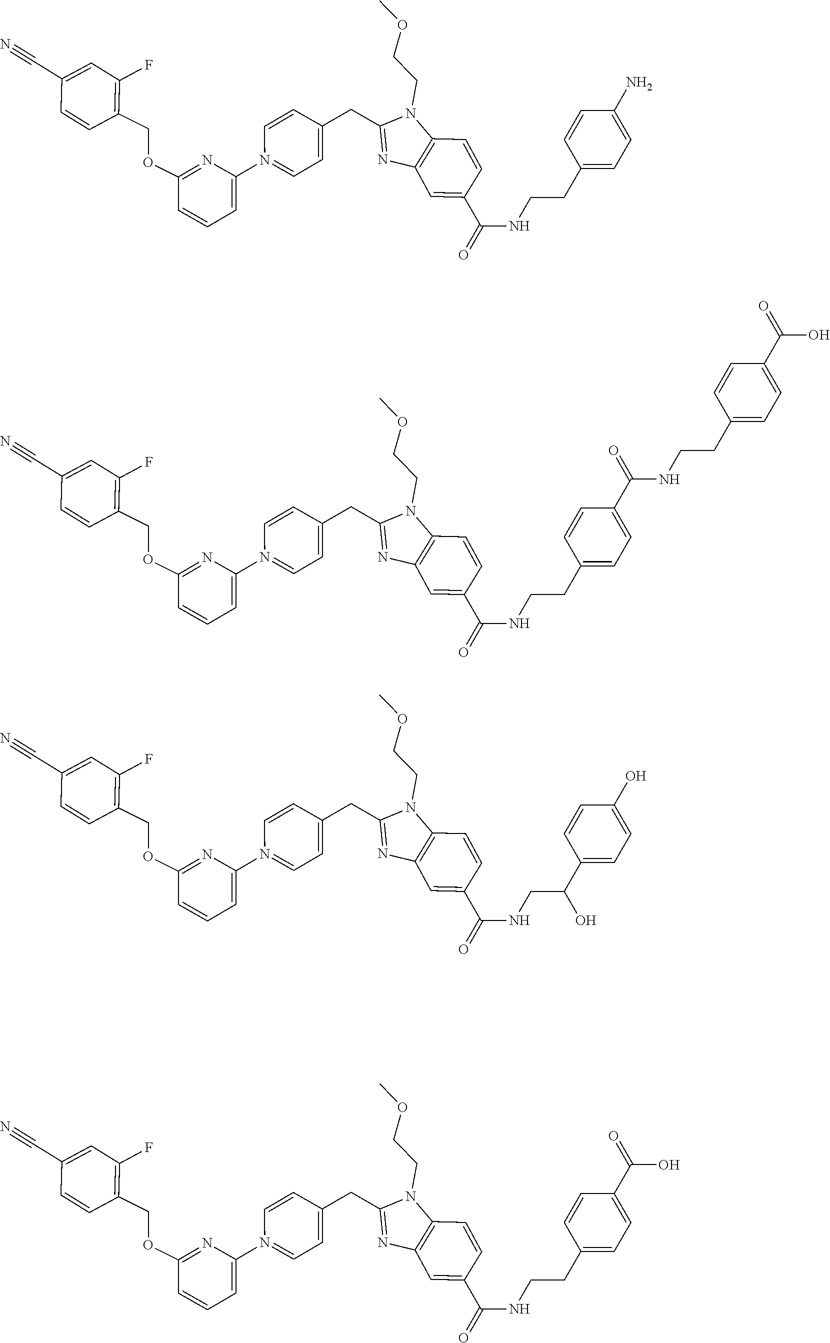

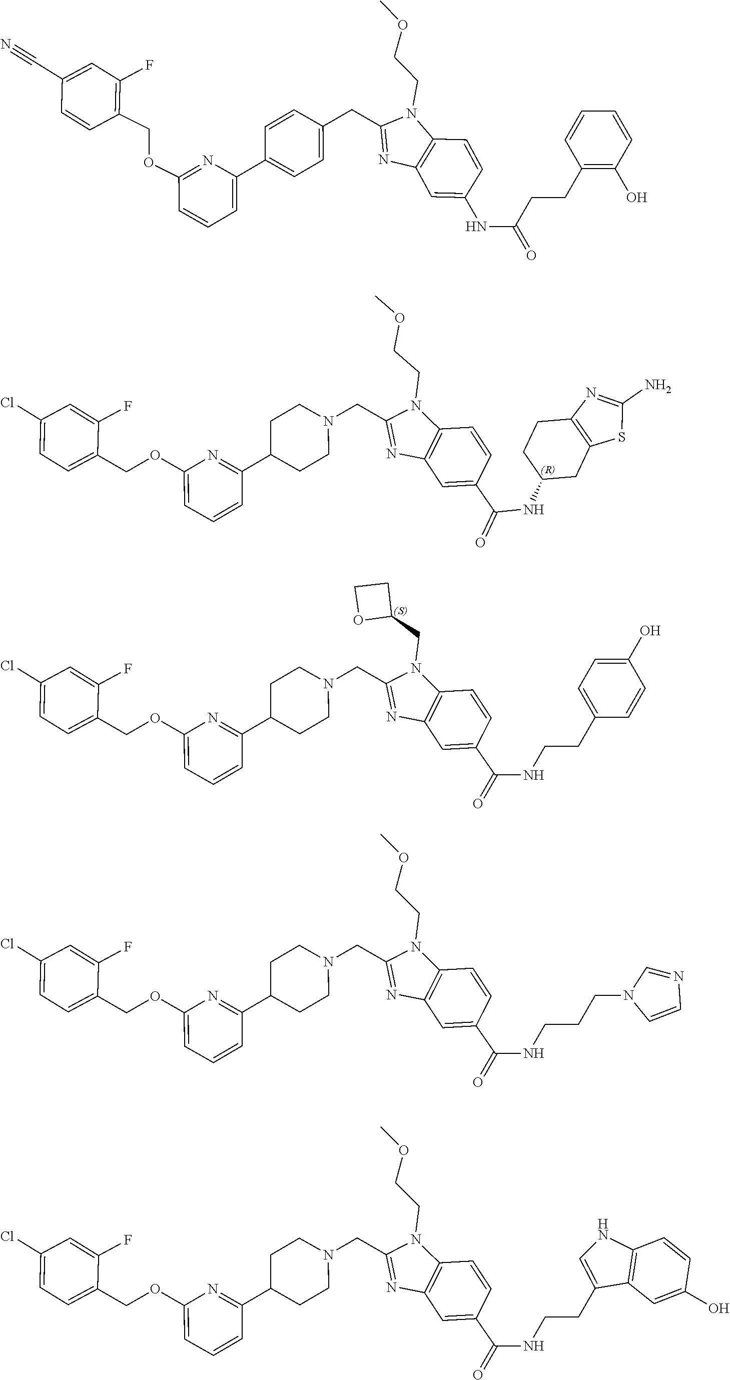

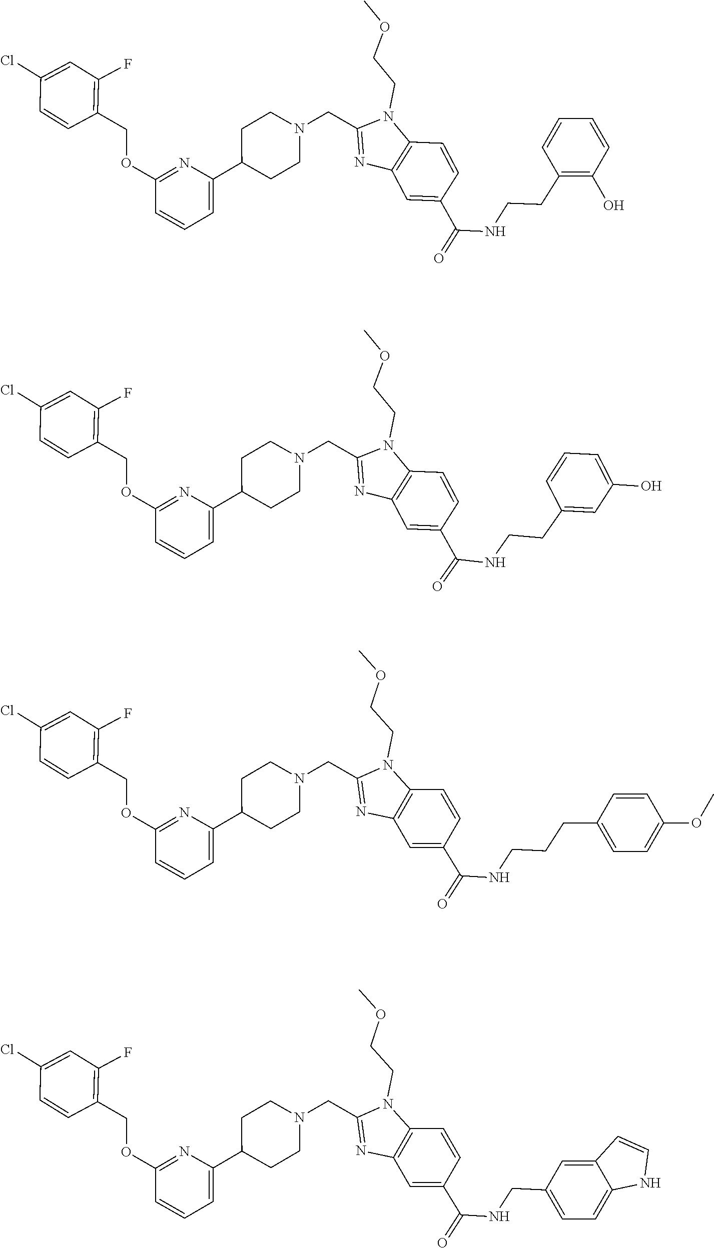 [SMILES: COCCn1c(CN2CCC(c3cccc(OCc4ccc(Cl)cc4F)n3)CC2)nc2cc(C(=O)NCCc3ccccc3O)ccc21 ; COCCn1c(CN2CCC(c3cccc(OCc4ccc(Cl)cc4F)n3)CC2)nc2cc(C(=O)NCCc3cccc(O)c3)ccc21 ; COCCn1c(CN2CCC(c3cccc(OCc4ccc(Cl)cc4F)n3)CC2)nc2cc(C(=O)NCCCc3ccc(OC)cc3)ccc21 ; COCCn1c(CN2CCC(c3cccc(OCc4ccc(Cl)cc4F)n3)CC2)nc2cc(C(=O)NCc3ccc4[nH]ccc4c3)ccc21]

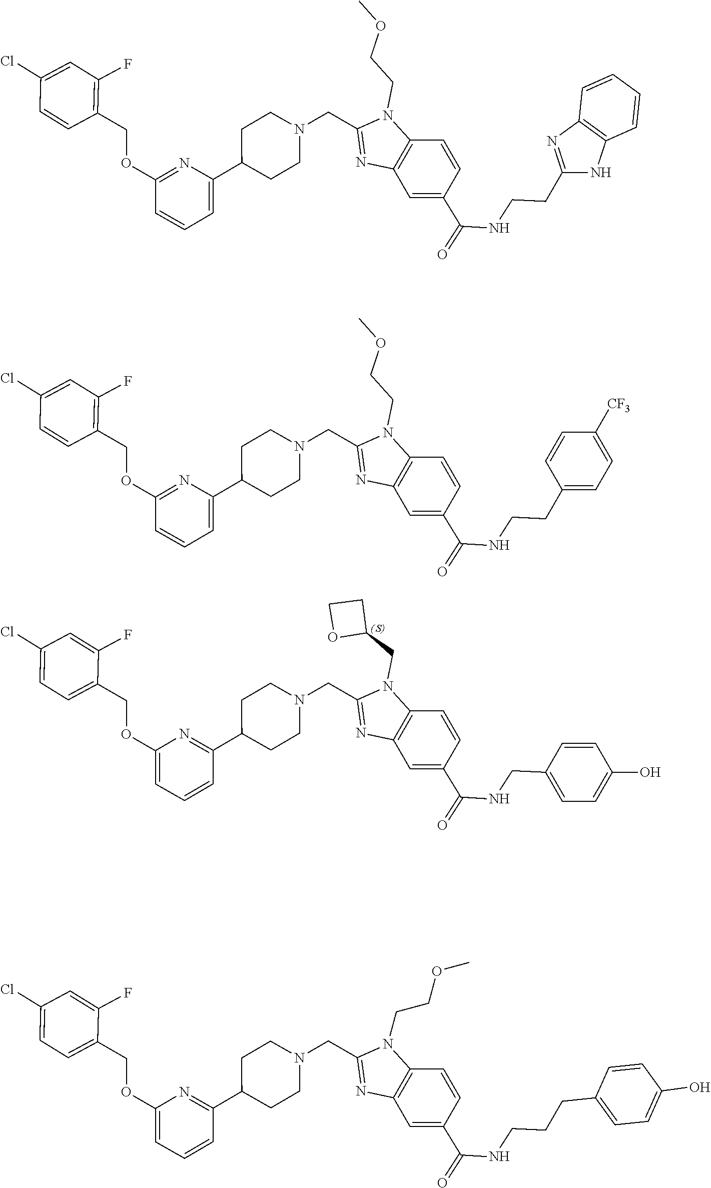

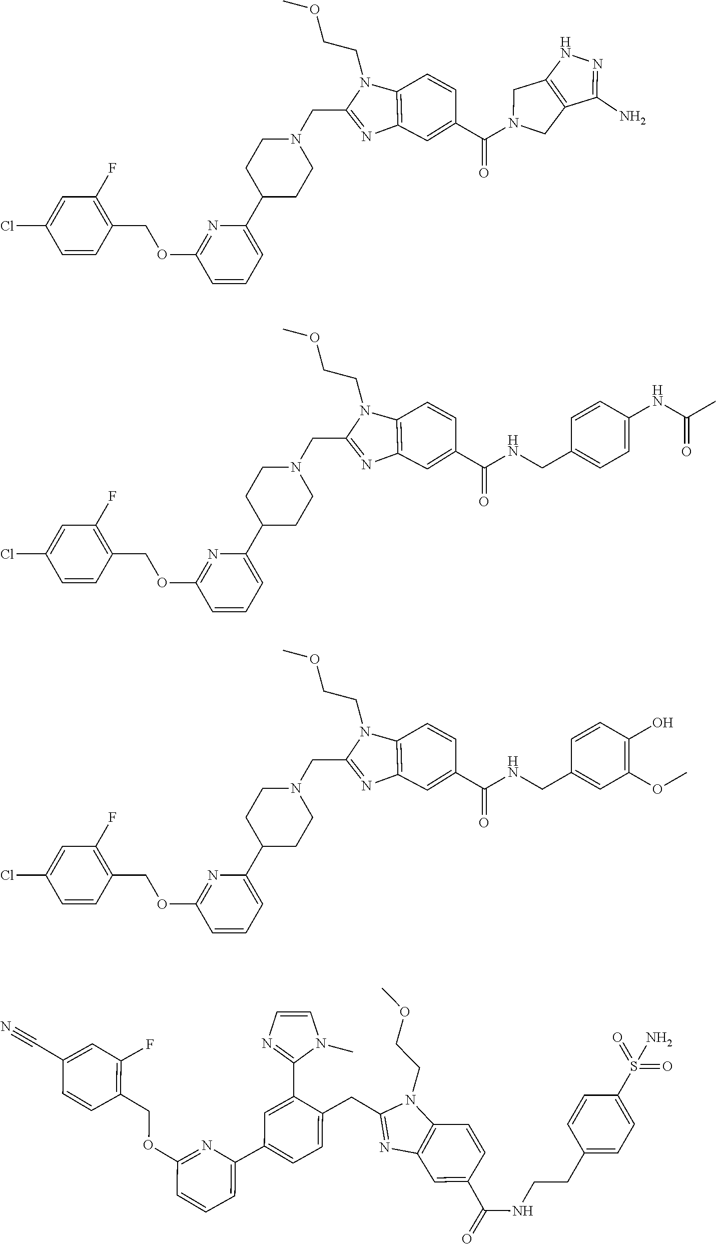 [SMILES: COCCn1c(CN2CCC(c3cccc(OCc4ccc(Cl)cc4F)n3)CC2)nc2cc(C(=O)N3Cc4[nH]nc(N)c4C3)ccc21 ; COCCn1c(CN2CCC(c3cccc(OCc4ccc(Cl)cc4F)n3)CC2)nc2cc(C(=O)NCc3ccc(NC(C)=O)cc3)ccc21 ; COCCn1c(CN2CCC(c3cccc(OCc4ccc(Cl)cc4F)n3)CC2)nc2cc(C(=O)NCc3ccc(O)c(OC)c3)ccc21 ; COCCn1c(Cc2ccc(-c3cccc(OCc4ccc(C#N)cc4F)n3)cc2-c2nccn2C)nc2cc(C(=O)NCCc3ccc(S(N)(=O)=O)cc3)ccc21]

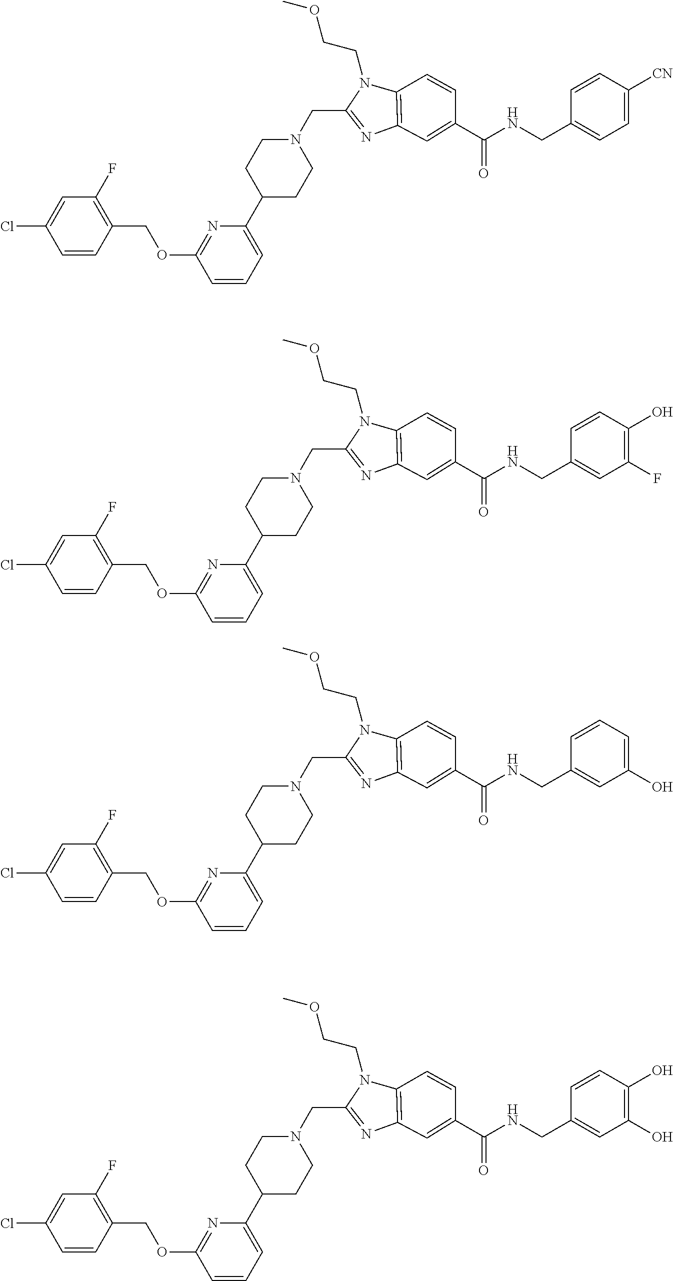

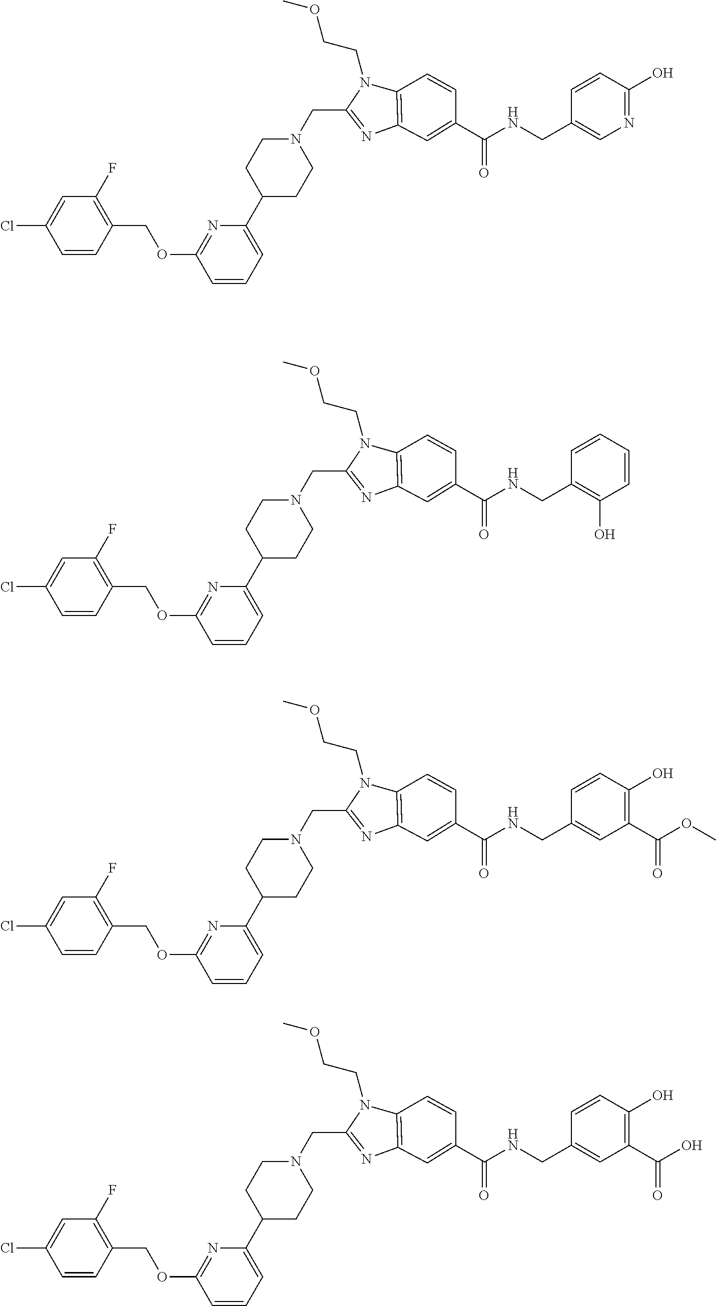 [SMILES: COCCn1c(CN2CCC(c3cccc(OCc4ccc(Cl)cc4F)n3)CC2)nc2cc(C(=O)NCc3ccc(O)nc3)ccc21 ; COCCn1c(CN2CCC(c3cccc(OCc4ccc(Cl)cc4F)n3)CC2)nc2cc(C(=O)NCc3ccccc3O)ccc21 ; COCCn1c(CN2CCC(c3cccc(OCc4ccc(Cl)cc4F)n3)CC2)nc2cc(C(=O)NCc3ccc(O)c(C(=O)OC)c3)ccc21 ; COCCn1c(CN2CCC(c3cccc(OCc4ccc(Cl)cc4F)n3)CC2)nc2cc(C(=O)NCc3ccc(O)c(C(=O)O)c3)ccc21]

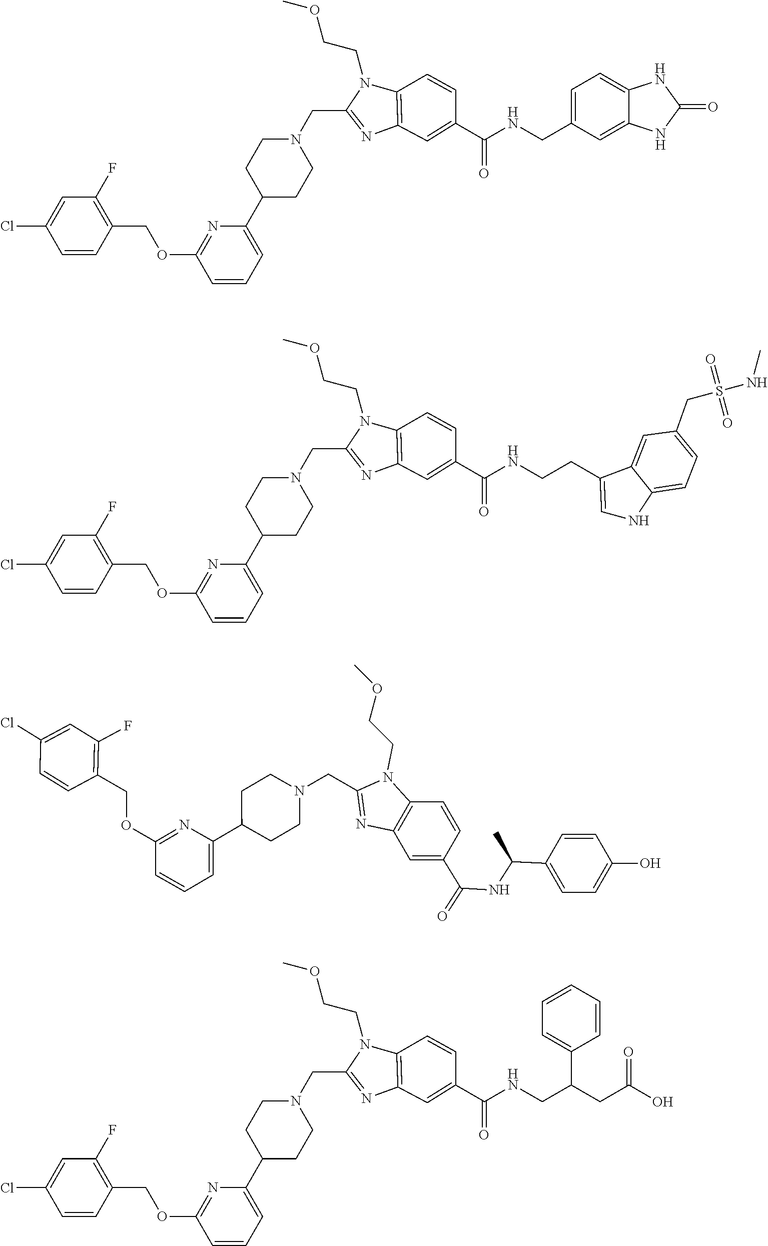 [SMILES: COCCn1c(CN2CCC(c3cccc(OCc4ccc(Cl)cc4F)n3)CC2)nc2cc(C(=O)NCc3ccc4[nH]c(=O)[nH]c4c3)ccc21 ; CNS(=O)(=O)Cc1ccc2[nH]cc(CCNC(=O)c3ccc4c(c3)nc(CN3CCC(c5cccc(OCc6ccc(Cl)cc6F)n5)CC3)n4CCOC)c2c1 ; COCCn1c(CN2CCC(c3cccc(OCc4ccc(Cl)cc4F)n3)CC2)nc2cc(C(=O)N[C@@H](C)c3ccc(O)cc3)ccc21 ; COCCn1c(CN2CCC(c3cccc(OCc4ccc(Cl)cc4F)n3)CC2)nc2cc(C(=O)NCC(CC(=O)O)c3ccccc3)ccc21]

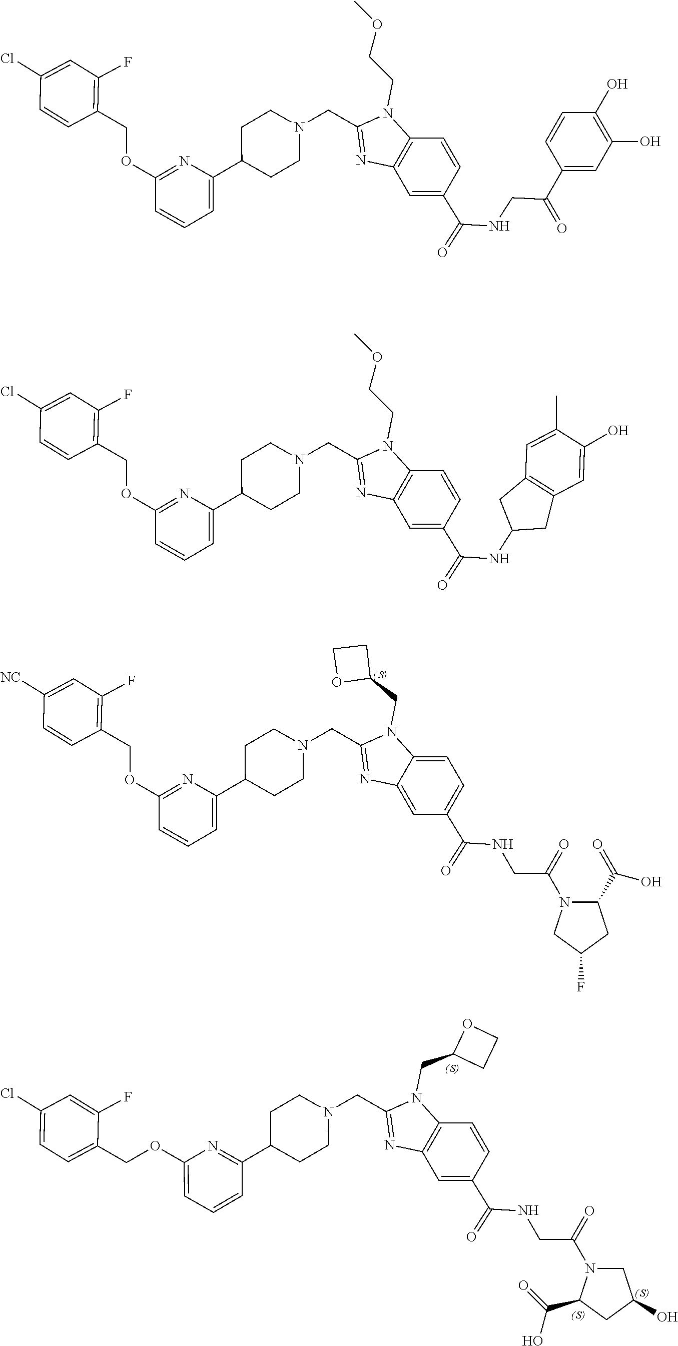

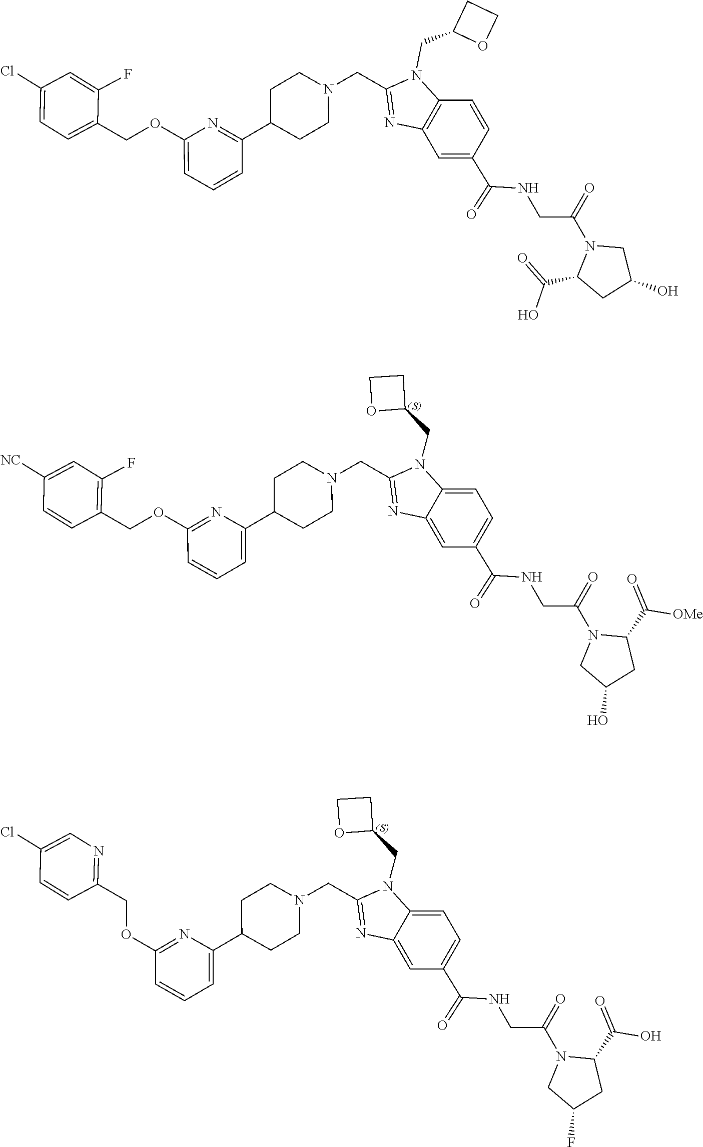

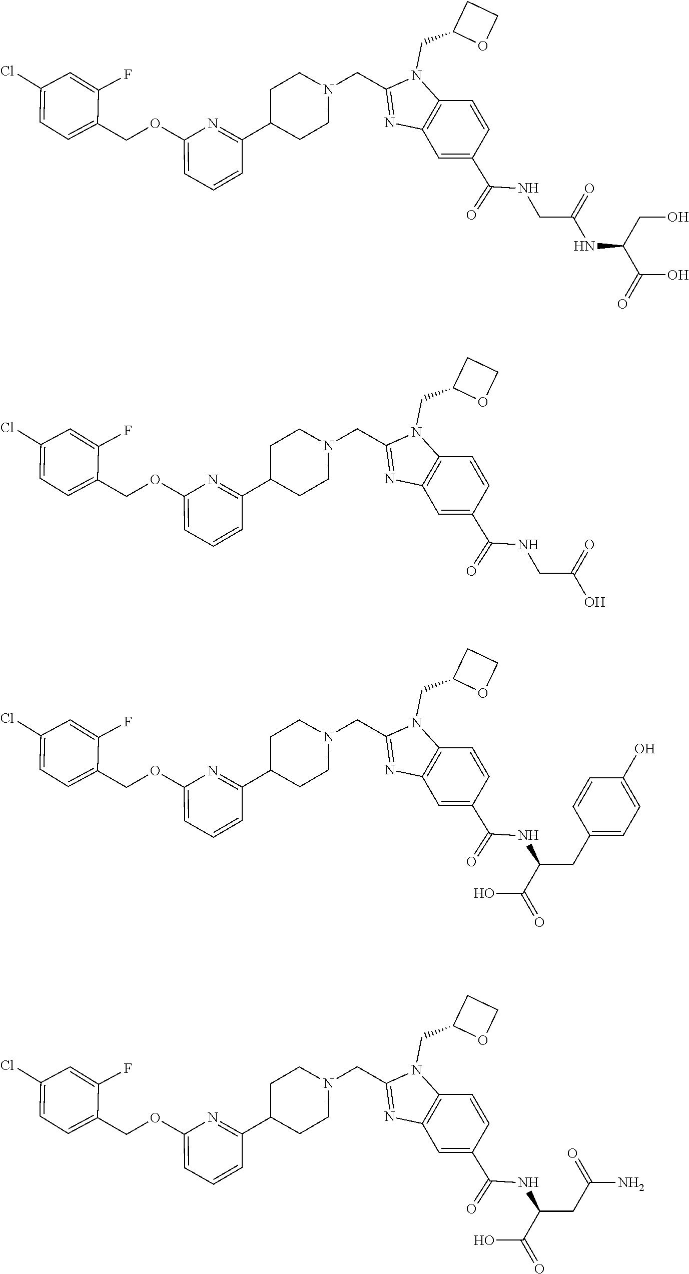 [SMILES: O=C(CNC(=O)c1ccc2c(c1)nc(CN1CCC(c3cccc(OCc4ccc(Cl)cc4F)n3)CC1)n2C[C@@H]1CCO1)N[C@@H](CO)C(=O)O ; O=C(O)CNC(=O)c1ccc2c(c1)nc(CN1CCC(c3cccc(OCc4ccc(Cl)cc4F)n3)CC1)n2C[C@@H]1CCO1 ; O=C(N[C@@H](Cc1ccc(O)cc1)C(=O)O)c1ccc2c(c1)nc(CN1CCC(c3cccc(OCc4ccc(Cl)cc4F)n3)CC1)n2C[C@@H]1CCO1 ; NC(=O)C[C@H](NC(=O)c1ccc2c(c1)nc(CN1CCC(c3cccc(OCc4ccc(Cl)cc4F)n3)CC1)n2C[C@@H]1CCO1)C(=O)O]

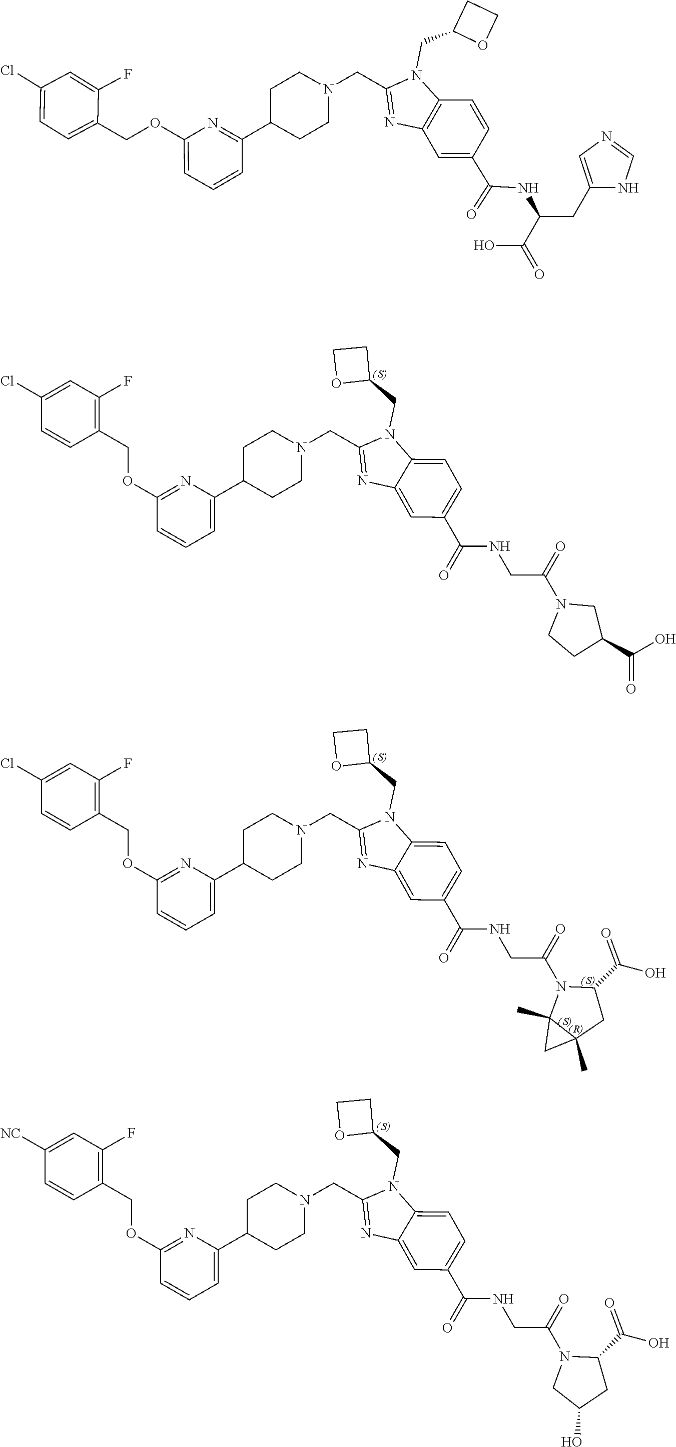

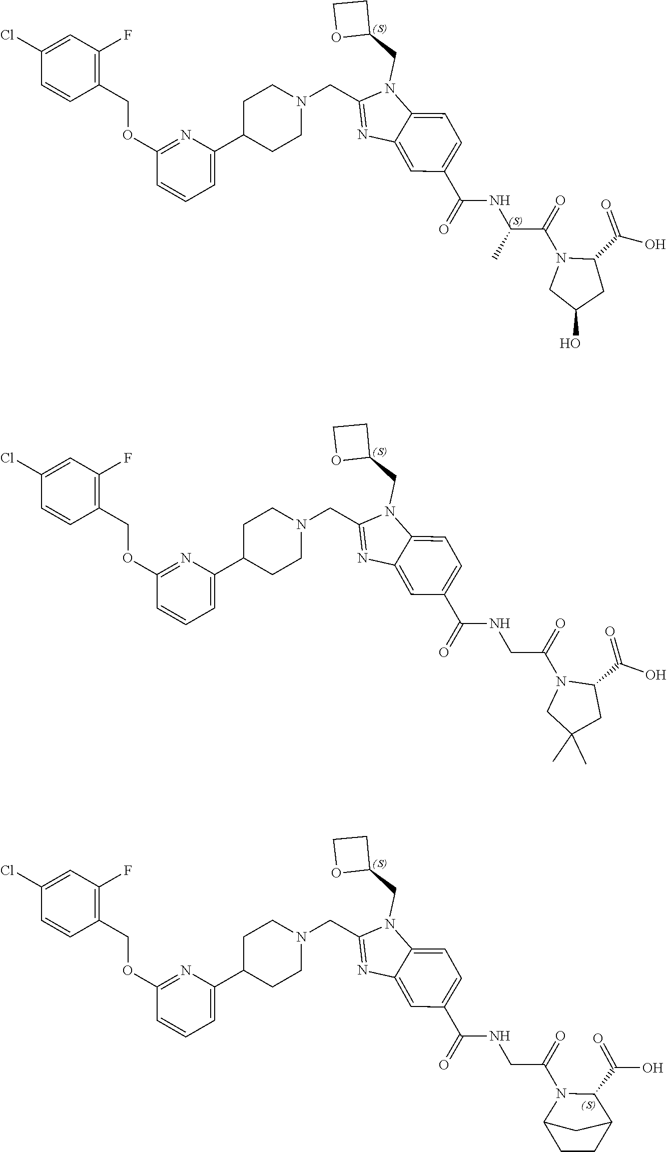

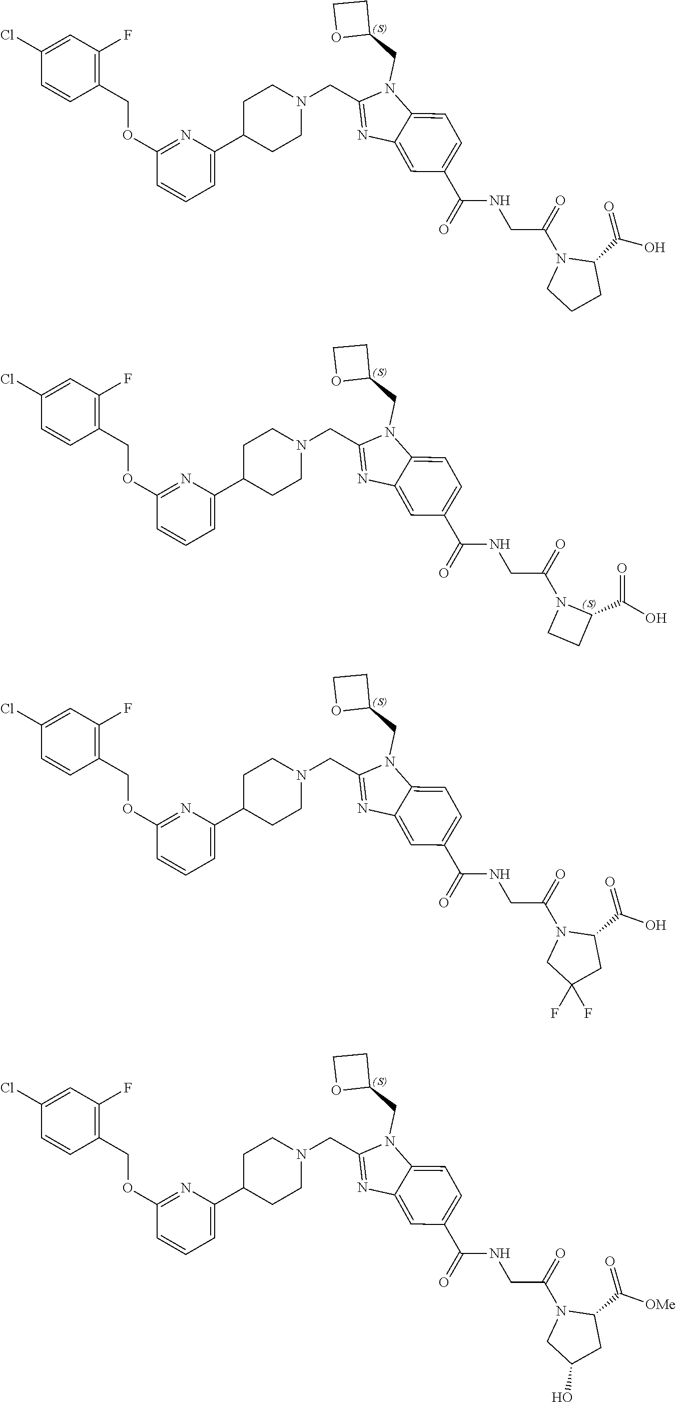

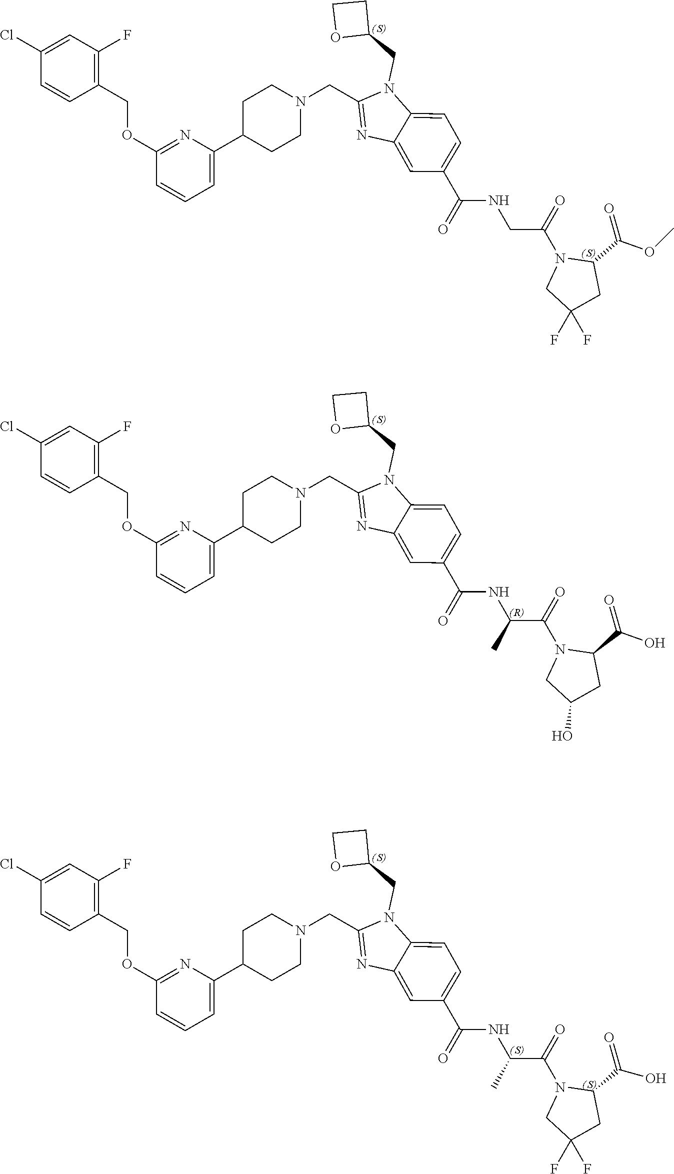

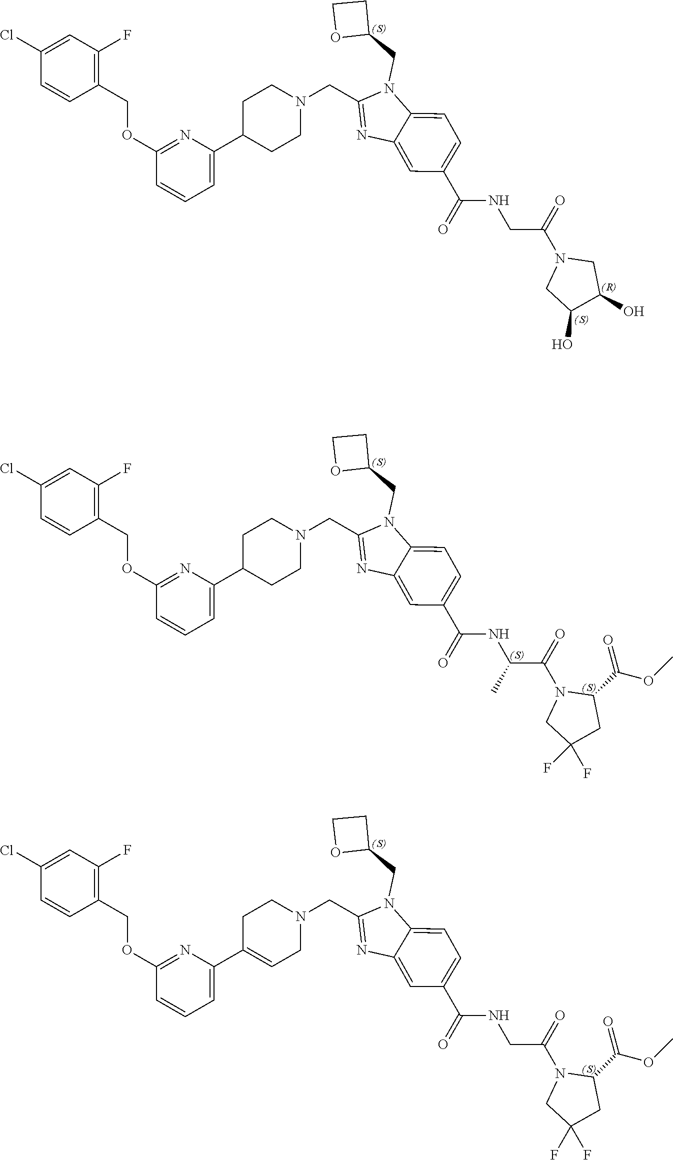

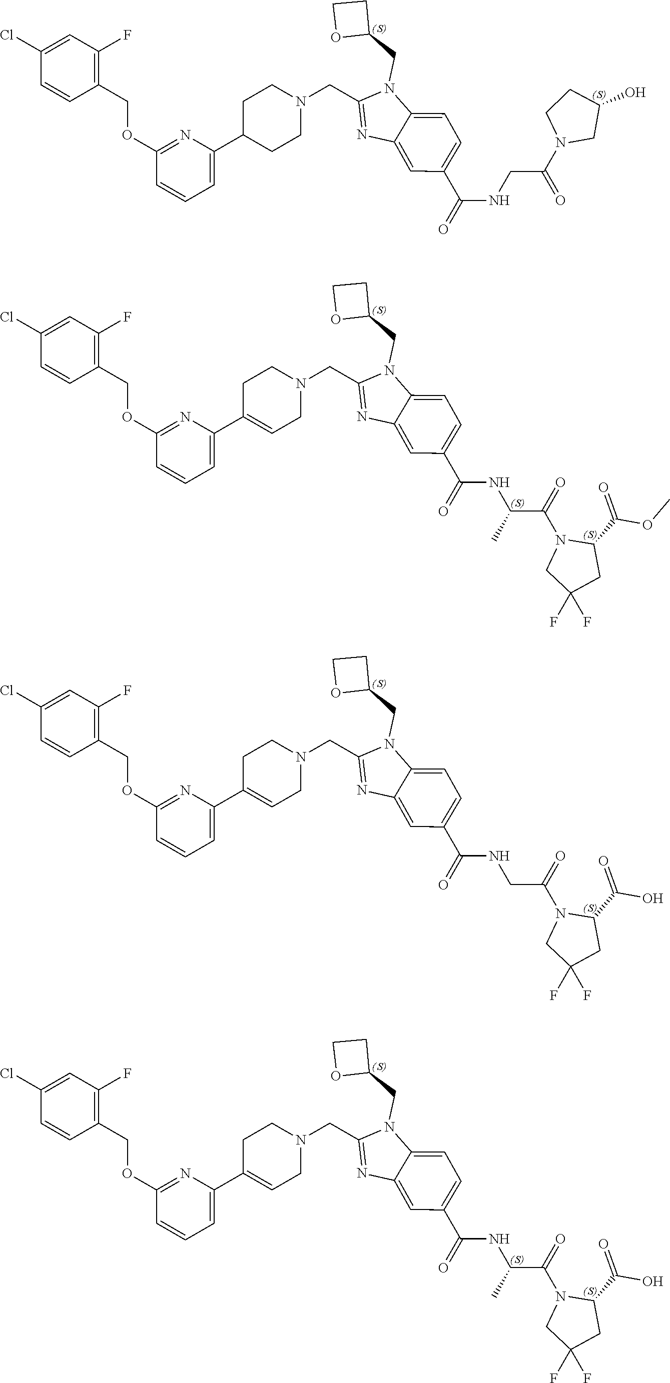

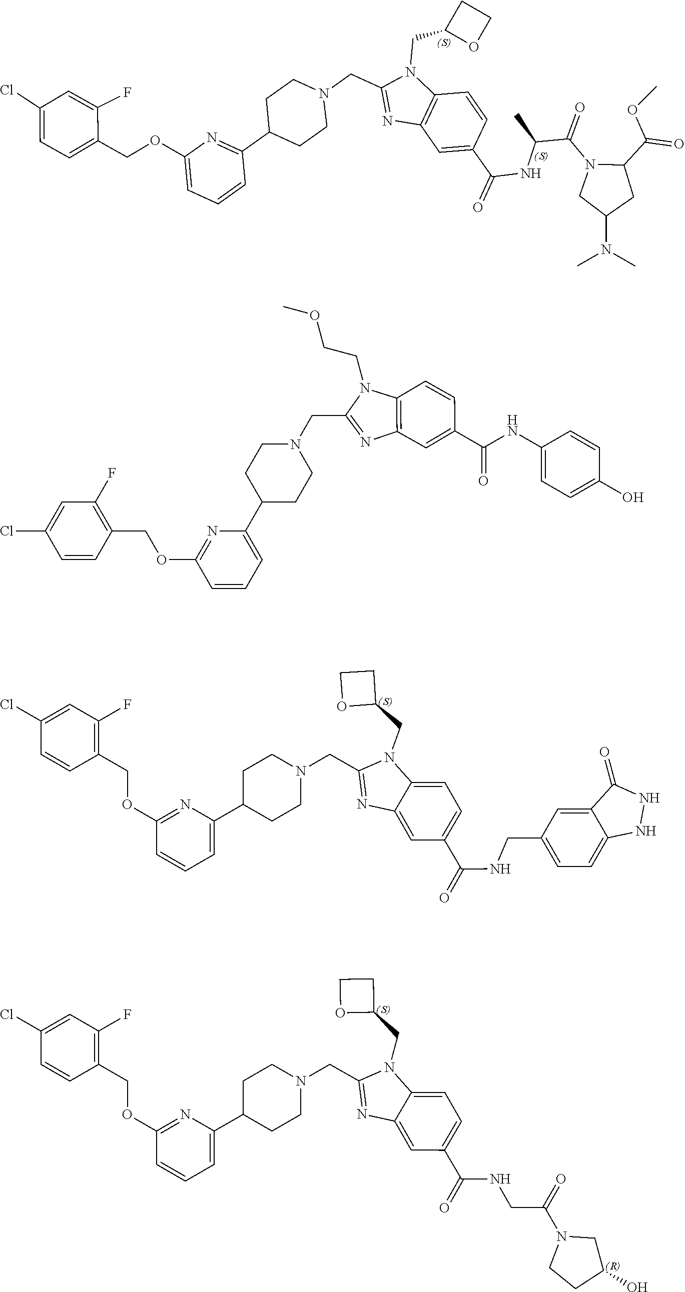

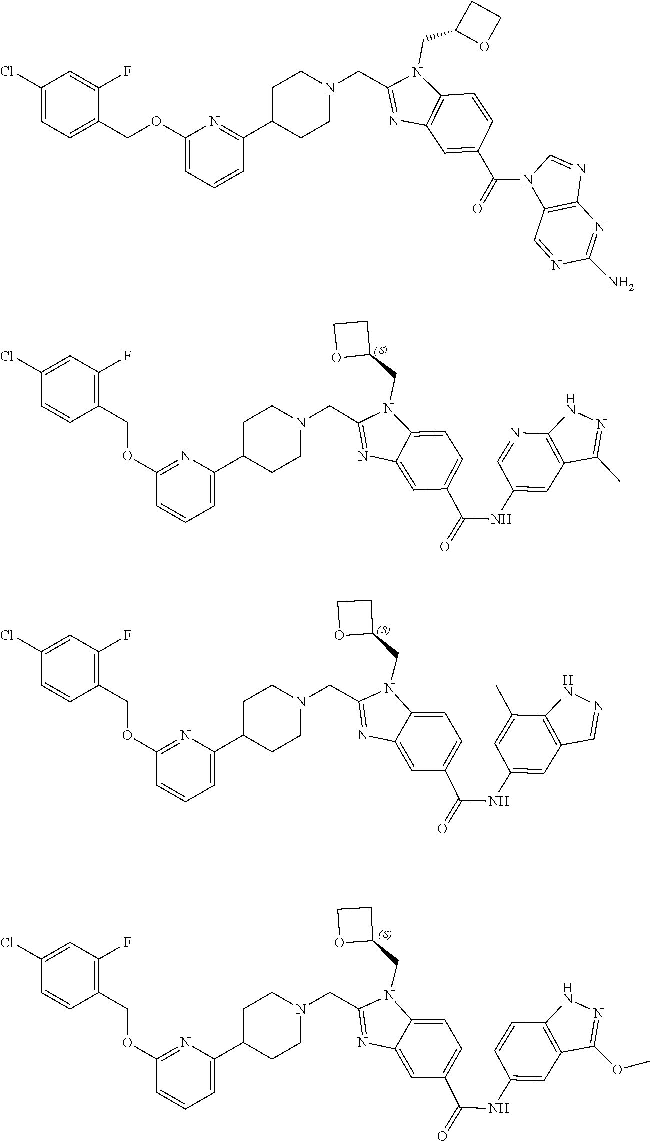

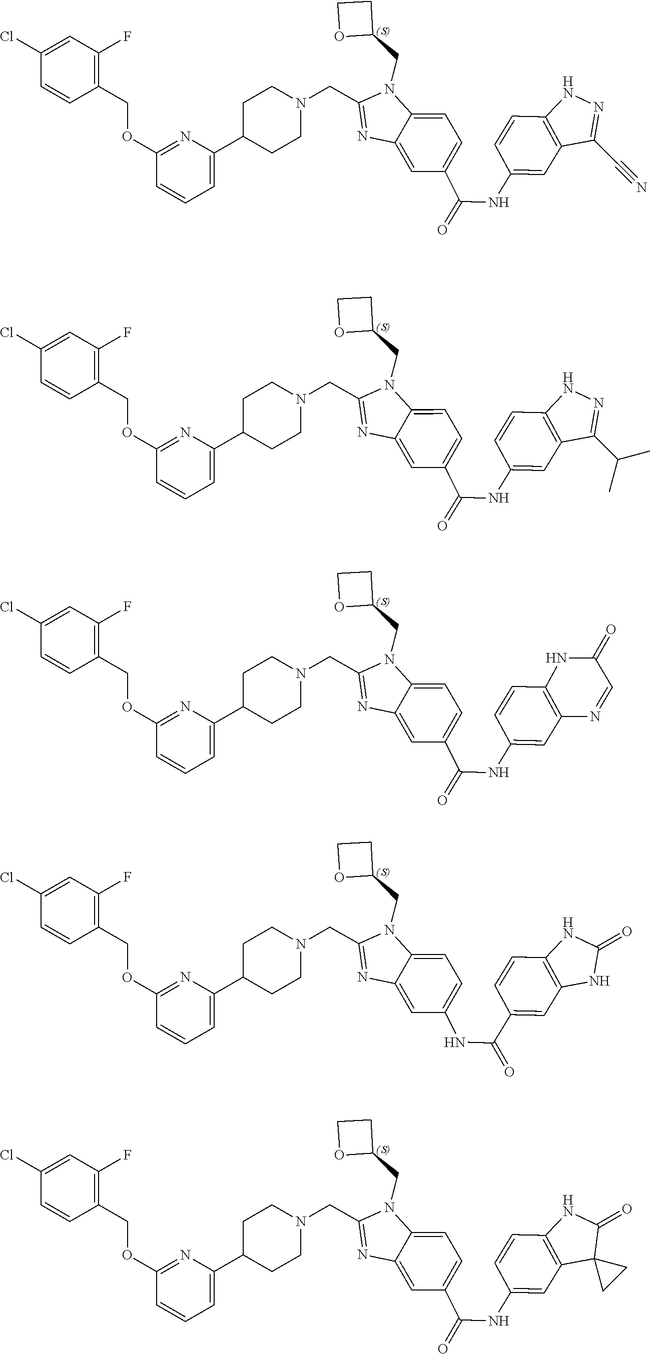

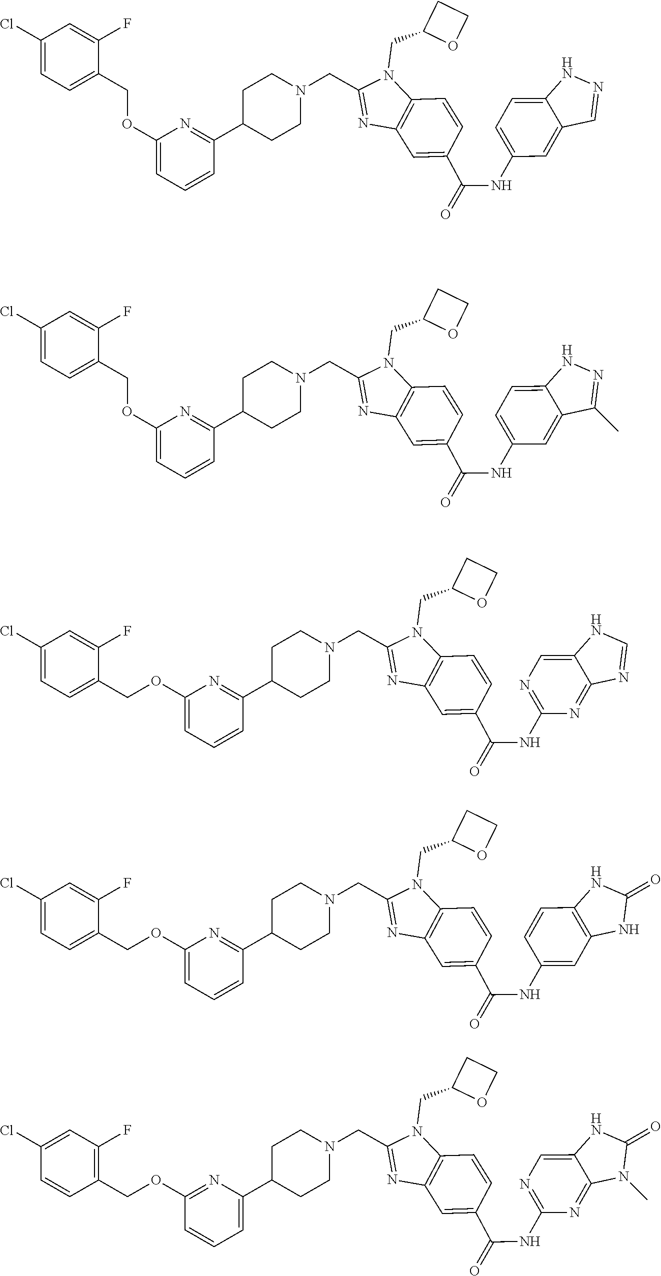

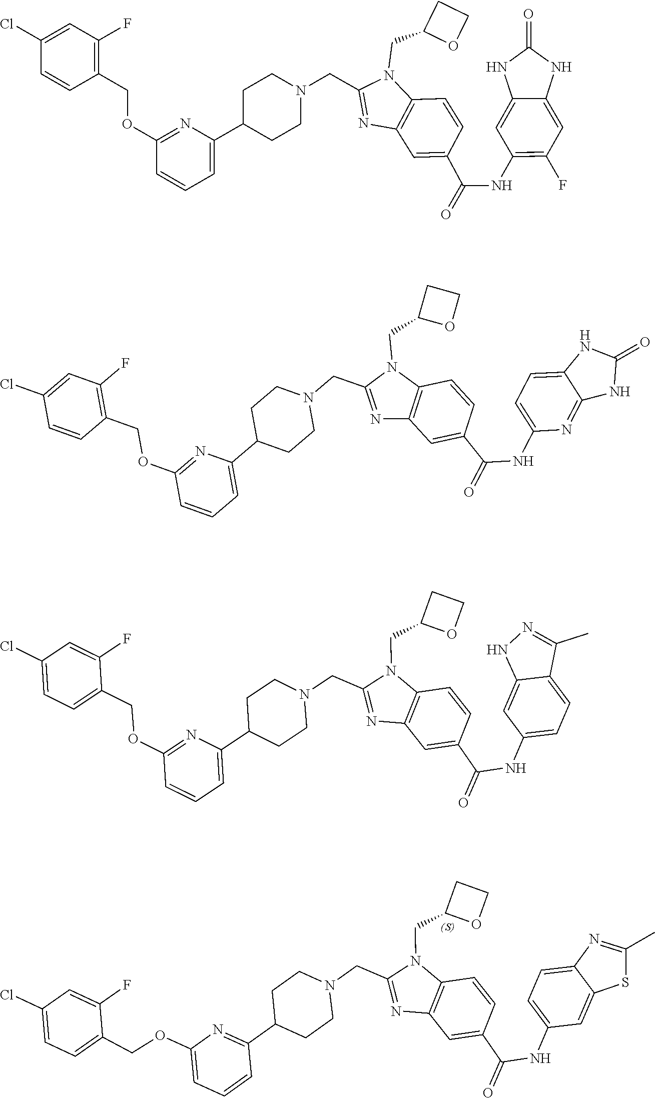

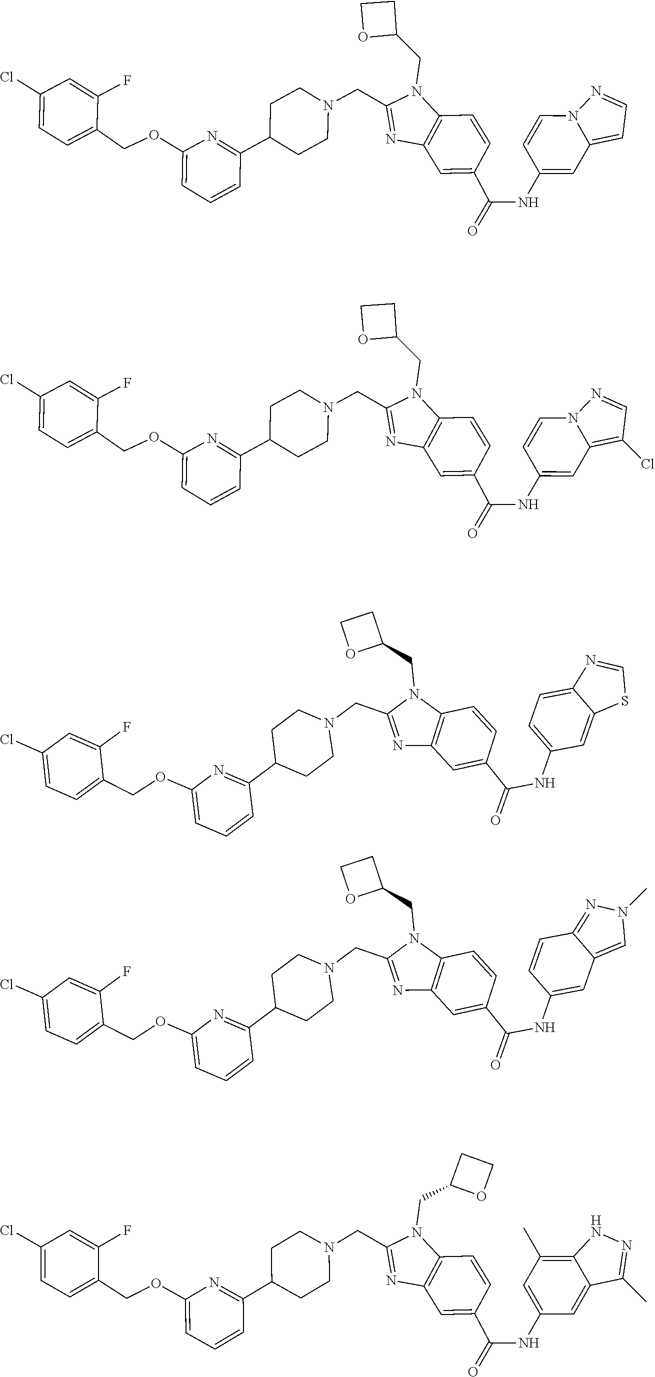 [SMILES: O=C(Nc1ccn2nccc2c1)c1ccc2c(c1)nc(CN1CCC(c3cccc(OCc4ccc(Cl)cc4F)n3)CC1)n2CC1CCO1 ; O=C(Nc1ccn2ncc(Cl)c2c1)c1ccc2c(c1)nc(CN1CCC(c3cccc(OCc4ccc(Cl)cc4F)n3)CC1)n2CC1CCO1 ; O=C(Nc1ccc2ncsc2c1)c1ccc2c(c1)nc(CN1CCC(c3cccc(OCc4ccc(Cl)cc4F)n3)CC1)n2C[C@@H]1CCO1 ; Cn1cc2cc(NC(=O)c3ccc4c(c3)nc(CN3CCC(c5cccc(OCc6ccc(Cl)cc6F)n5)CC3)n4C[C@@H]3CCO3)ccc2n1 ; Cc1n[nH]c2c(C)cc(NC(=O)c3ccc4c(c3)nc(CN3CCC(c5cccc(OCc6ccc(Cl)cc6F)n5)CC3)n4C[C@@H]3CCO3)cc12]

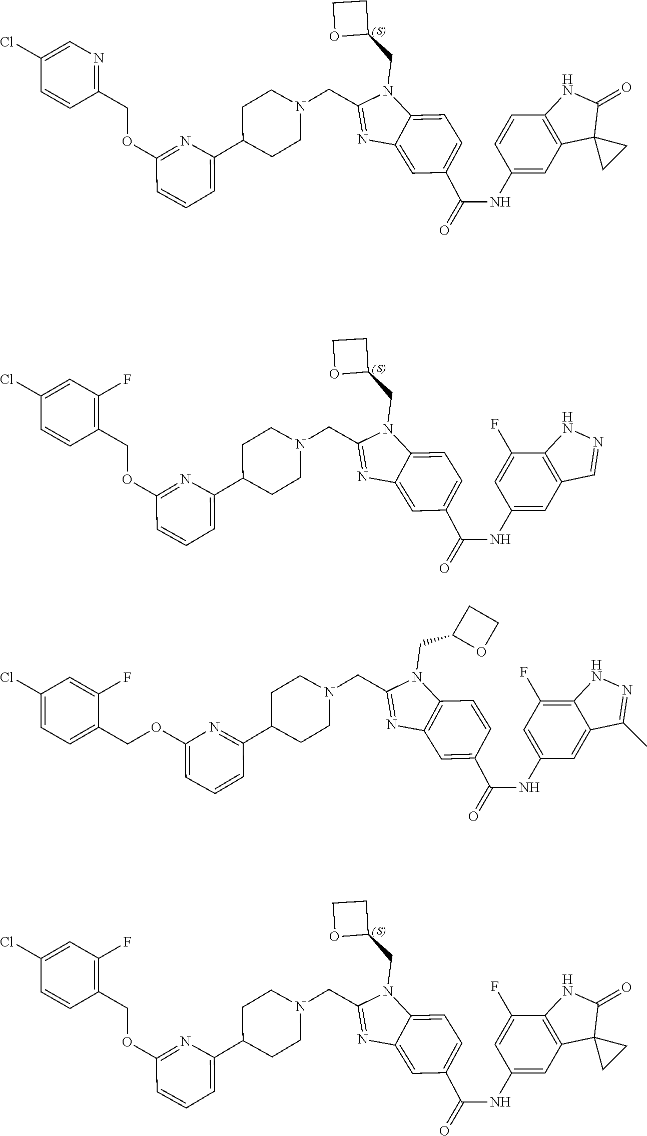

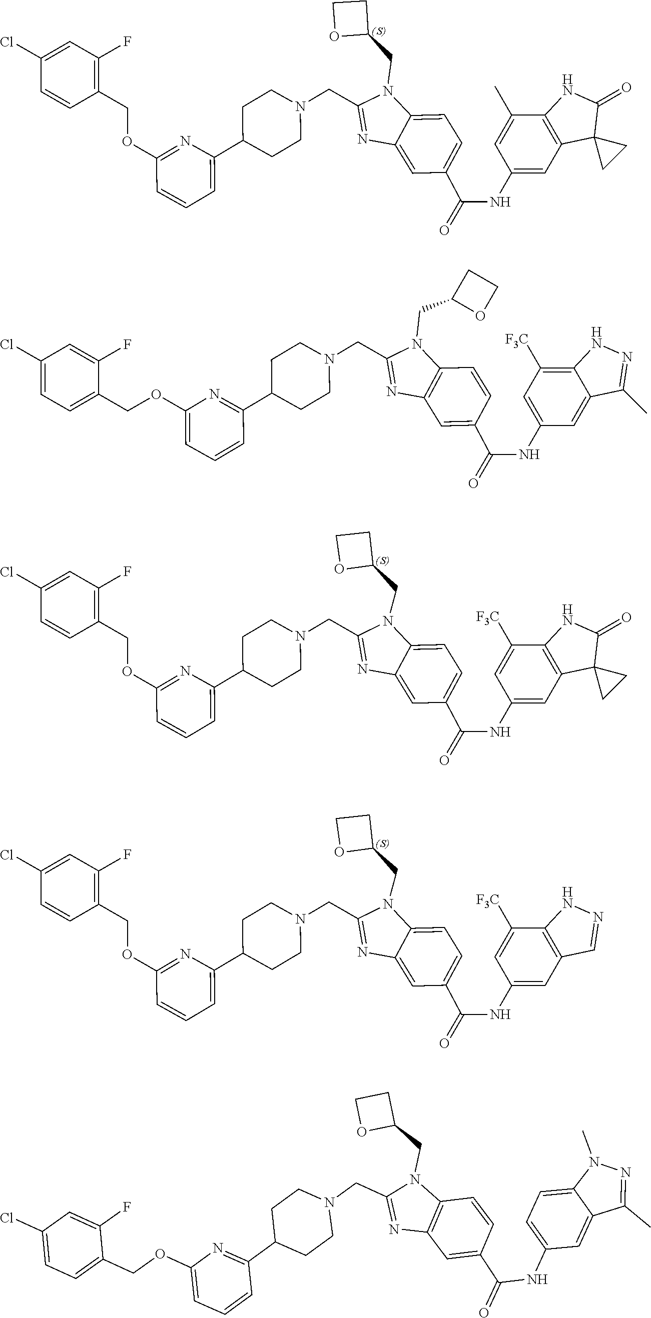

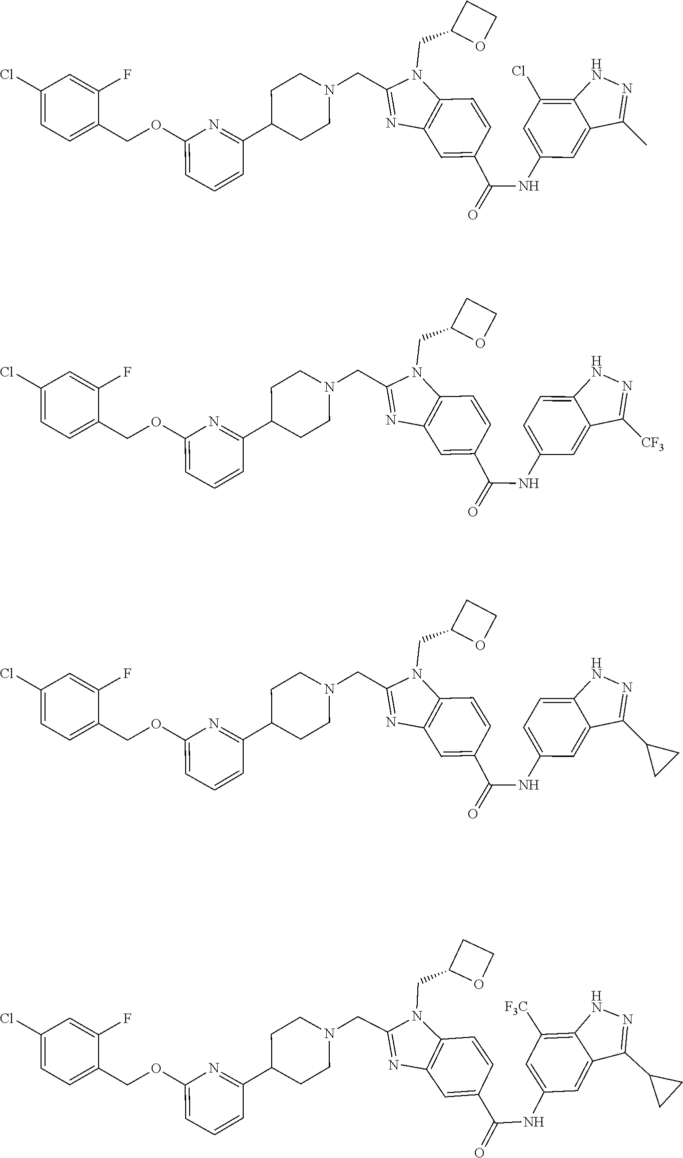 [SMILES: Cc1n[nH]c2c(Cl)cc(NC(=O)c3ccc4c(c3)nc(CN3CCC(c5cccc(OCc6ccc(Cl)cc6F)n5)CC3)n4C[C@@H]3CCO3)cc12 ; O=C(Nc1ccc2[nH]nc(C(F)(F)F)c2c1)c1ccc2c(c1)nc(CN1CCC(c3cccc(OCc4ccc(Cl)cc4F)n3)CC1)n2C[C@@H]1CCO1 ; O=C(Nc1ccc2[nH]nc(C3CC3)c2c1)c1ccc2c(c1)nc(CN1CCC(c3cccc(OCc4ccc(Cl)cc4F)n3)CC1)n2C[C@@H]1CCO1 ; O=C(Nc1cc(C(F)(F)F)c2[nH]nc(C3CC3)c2c1)c1ccc2c(c1)nc(CN1CCC(c3cccc(OCc4ccc(Cl)cc4F)n3)CC1)n2C[C@@H]1CCO1]

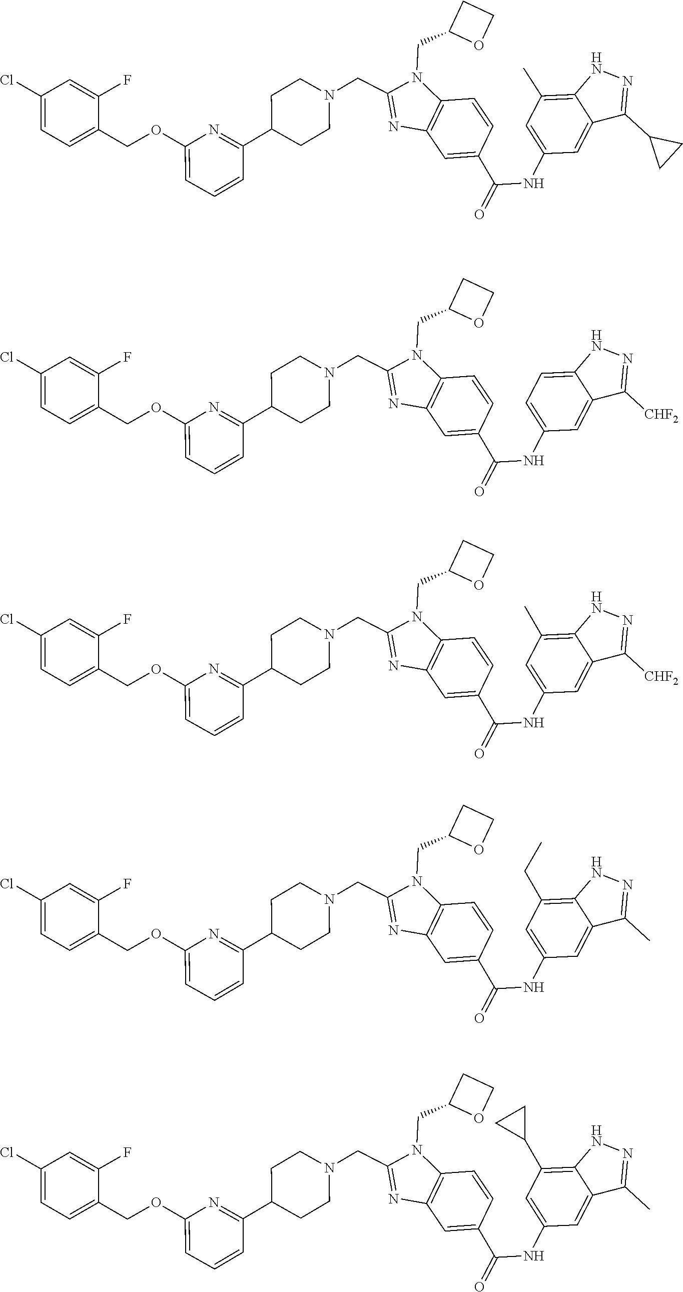 [SMILES: Cc1cc(NC(=O)c2ccc3c(c2)nc(CN2CCC(c4cccc(OCc5ccc(Cl)cc5F)n4)CC2)n3C[C@@H]2CCO2)cc2c(C3CC3)n[nH]c12 ; O=C(Nc1ccc2[nH]nc(C(F)F)c2c1)c1ccc2c(c1)nc(CN1CCC(c3cccc(OCc4ccc(Cl)cc4F)n3)CC1)n2C[C@@H]1CCO1 ; Cc1cc(NC(=O)c2ccc3c(c2)nc(CN2CCC(c4cccc(OCc5ccc(Cl)cc5F)n4)CC2)n3C[C@@H]2CCO2)cc2c(C(F)F)n[nH]c12 ; CCc1cc(NC(=O)c2ccc3c(c2)nc(CN2CCC(c4cccc(OCc5ccc(Cl)cc5F)n4)CC2)n3C[C@@H]2CCO2)cc2c(C)n[nH]c12 ; Cc1n[nH]c2c(C3CC3)cc(NC(=O)c3ccc4c(c3)nc(CN3CCC(c5cccc(OCc6ccc(Cl)cc6F)n5)CC3)n4C[C@@H]3CCO3)cc12]

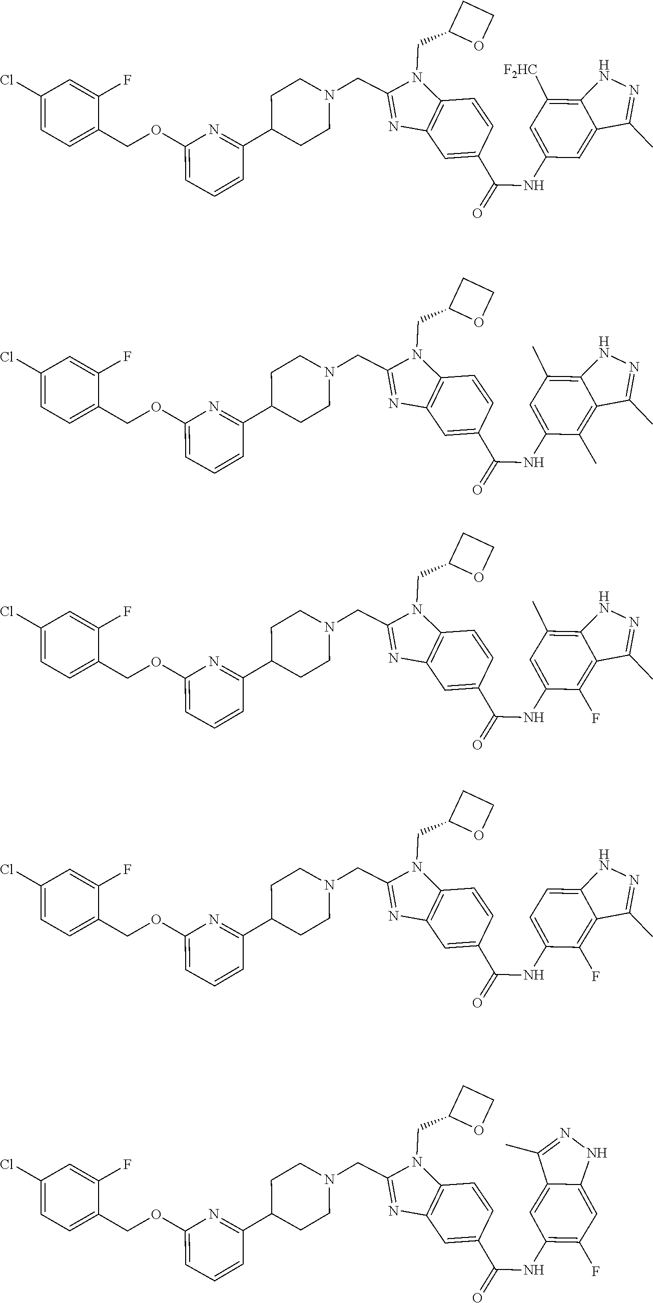

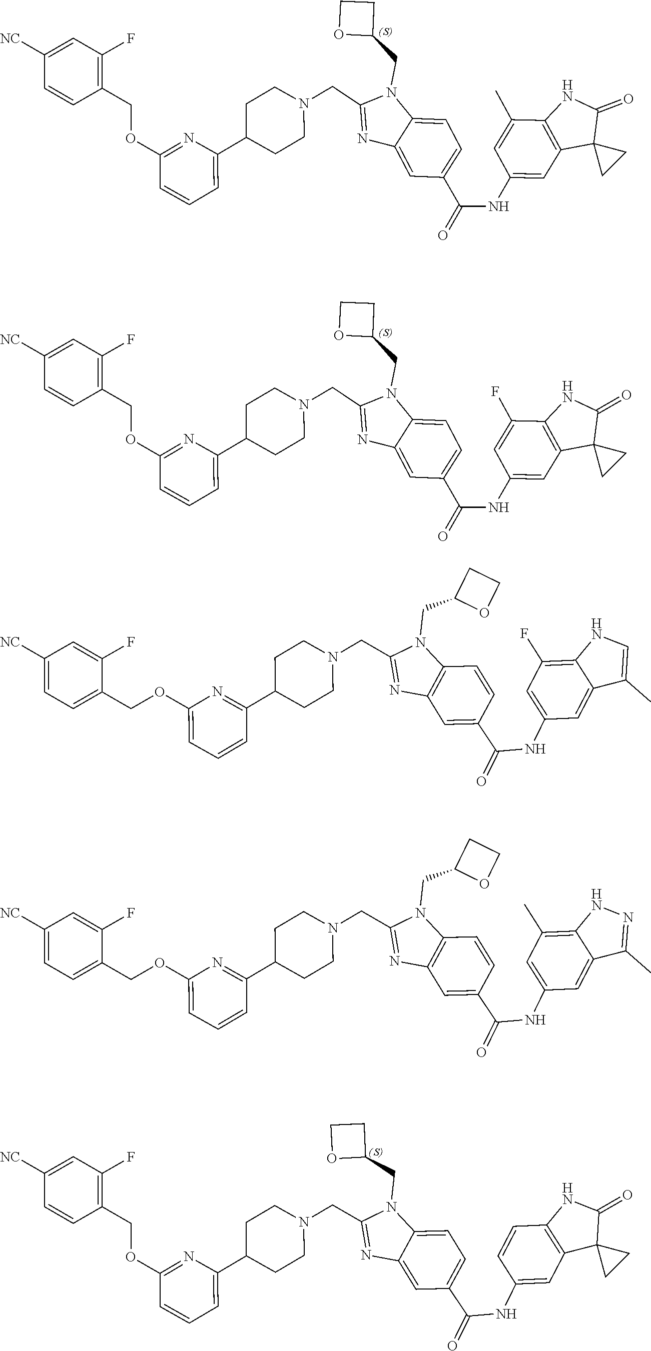

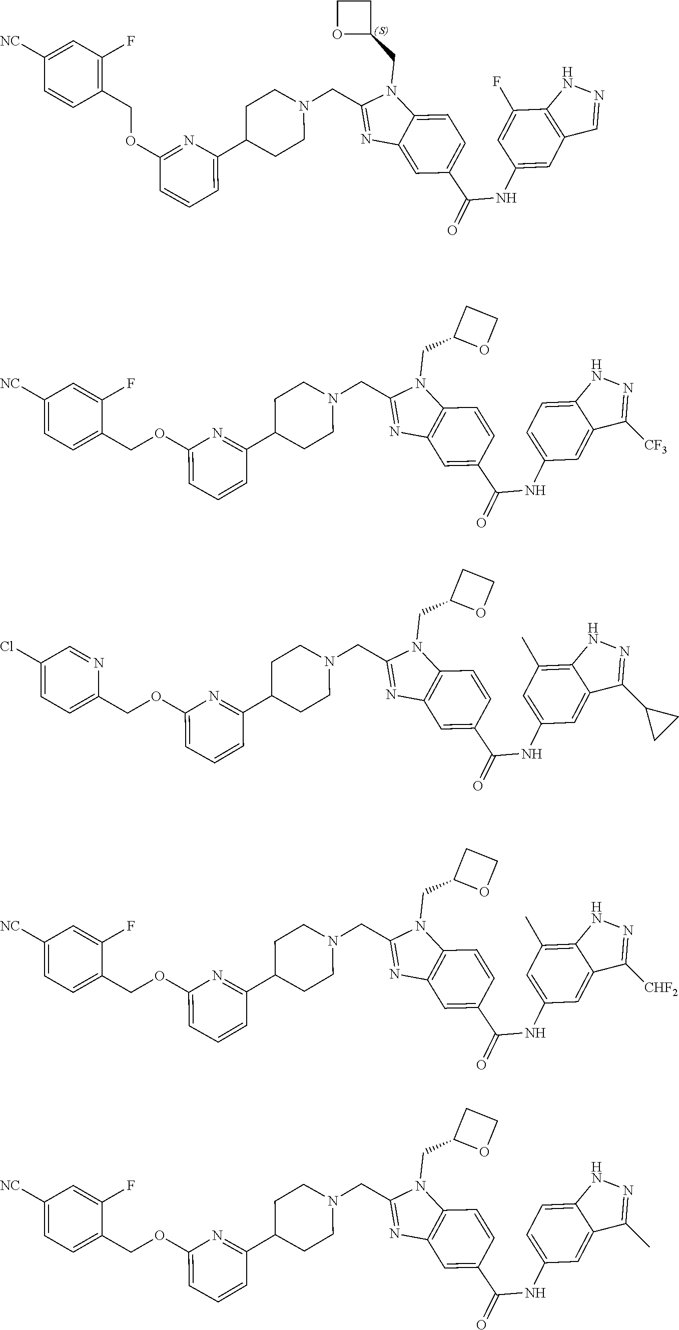

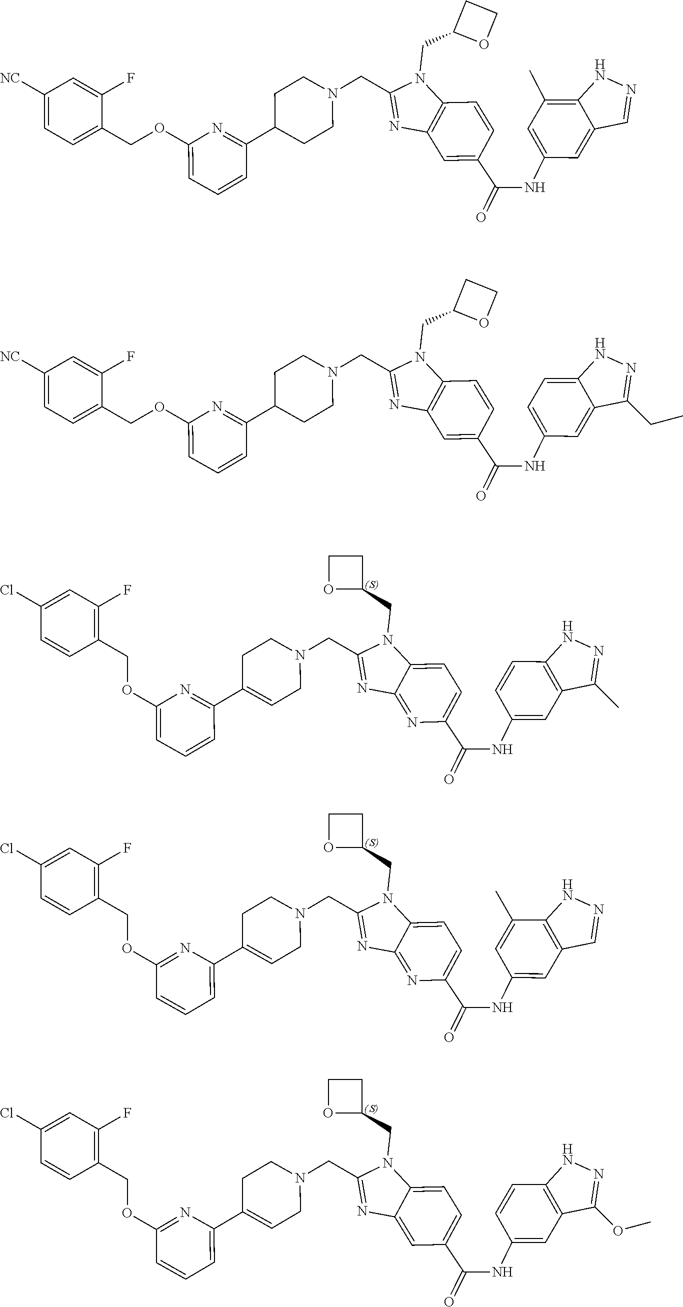

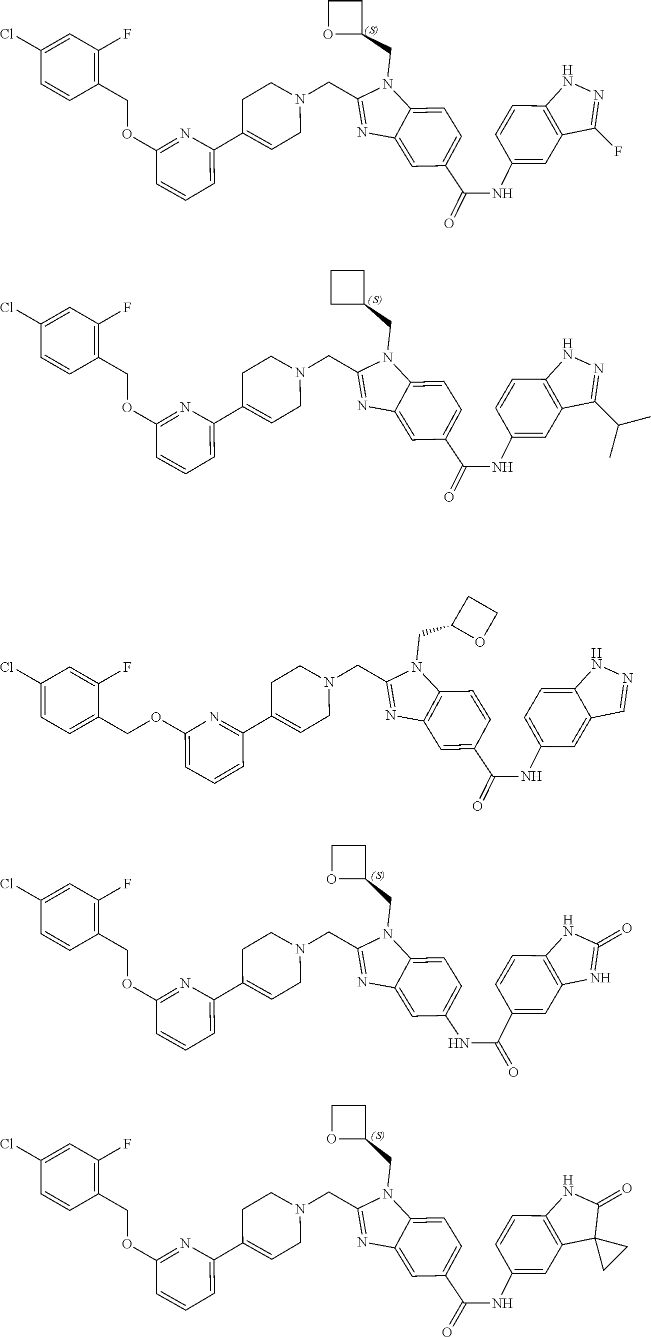

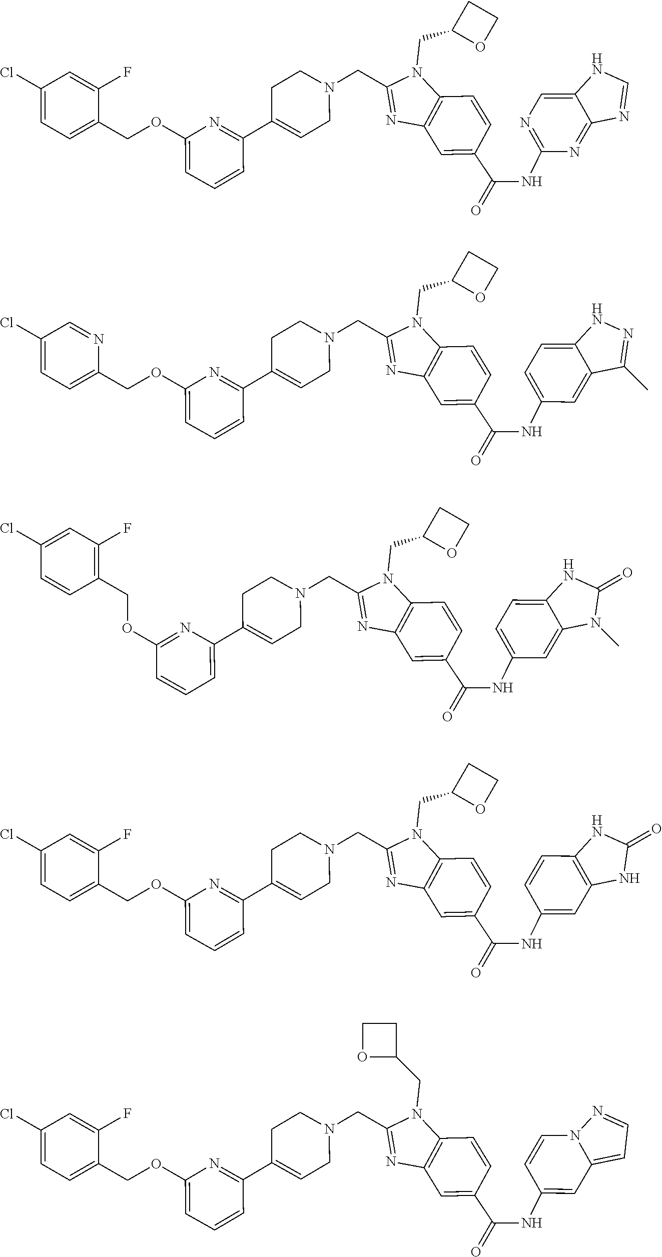

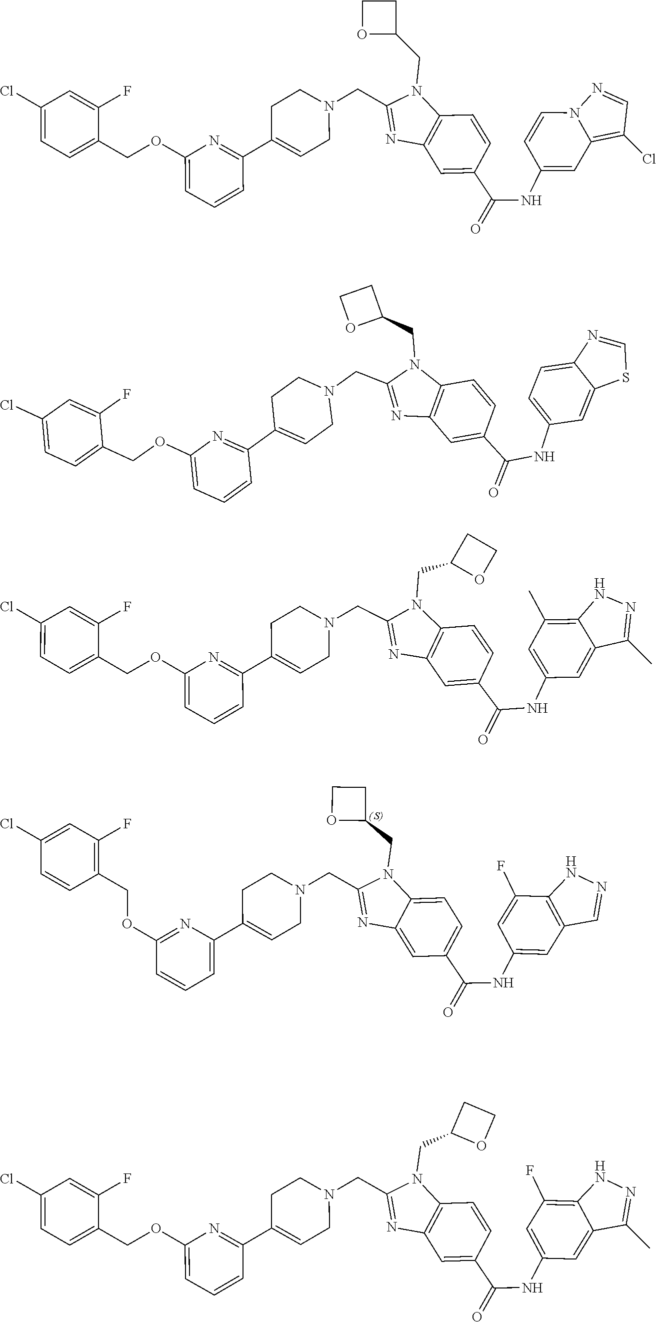

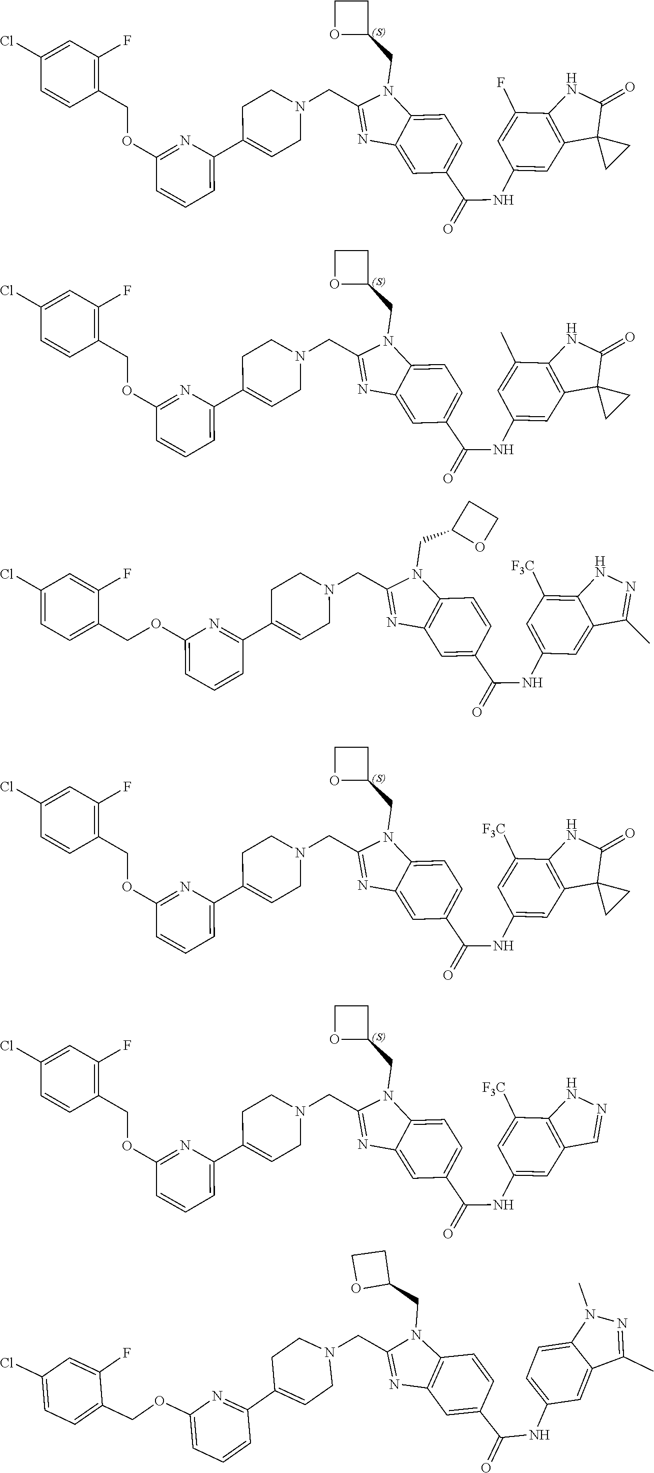

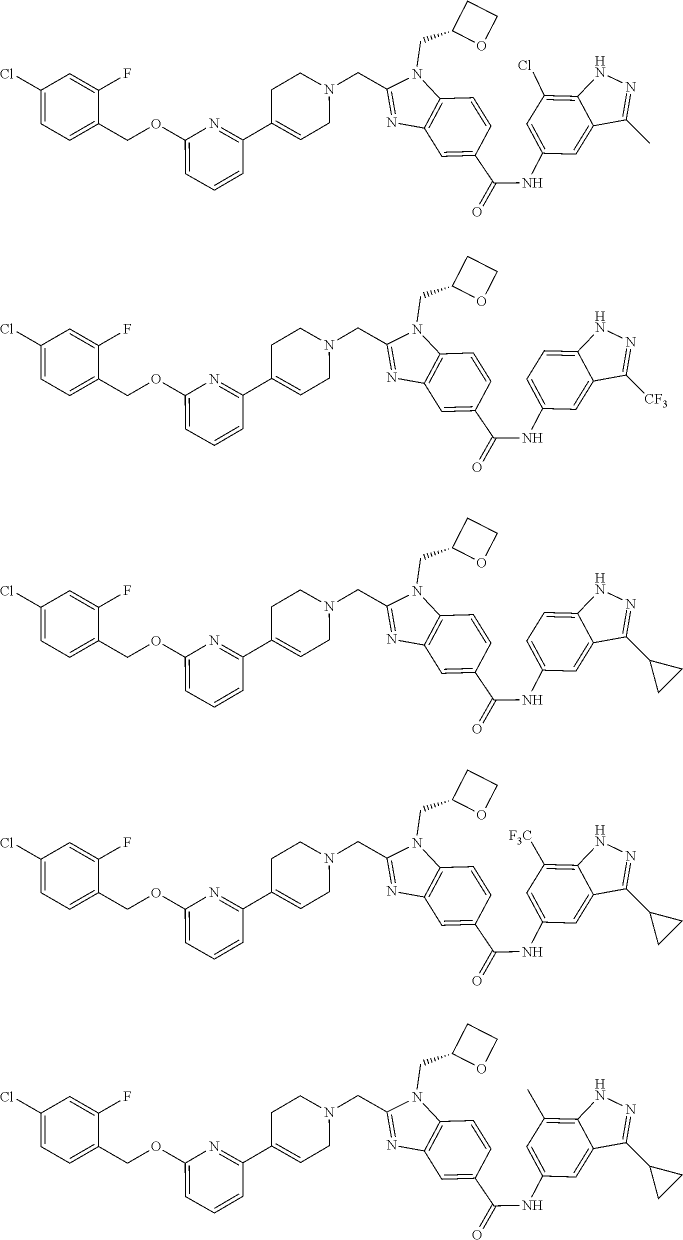

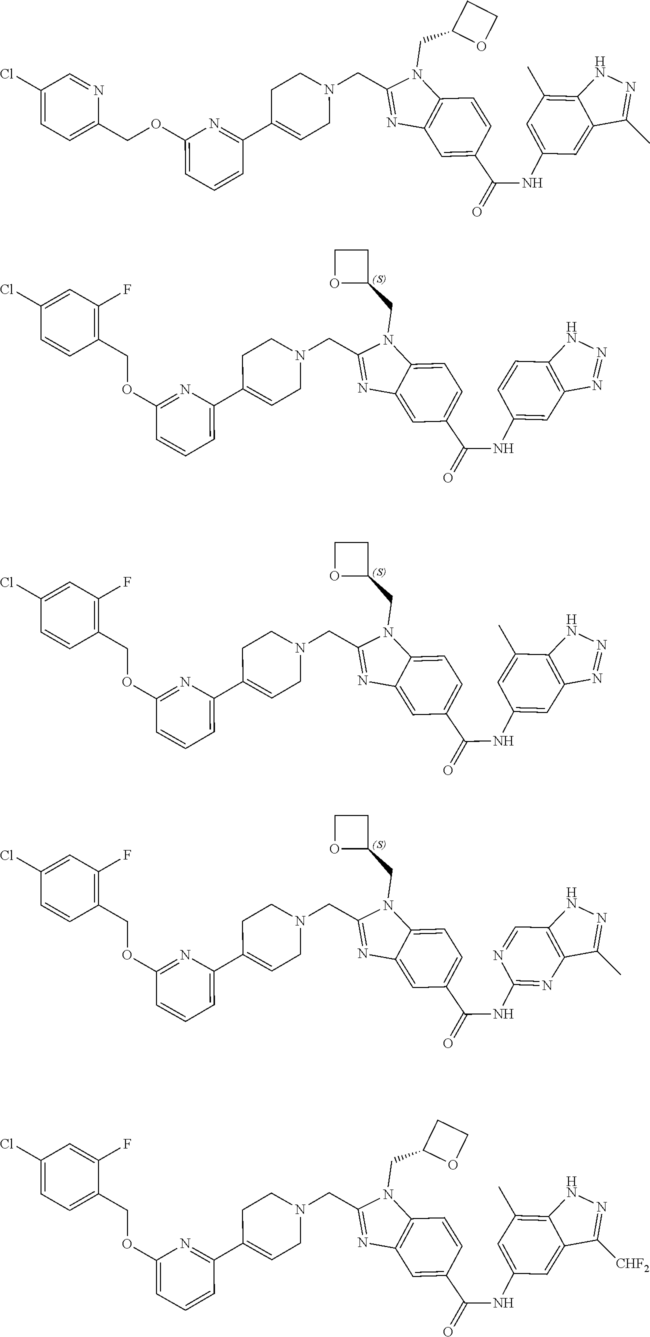

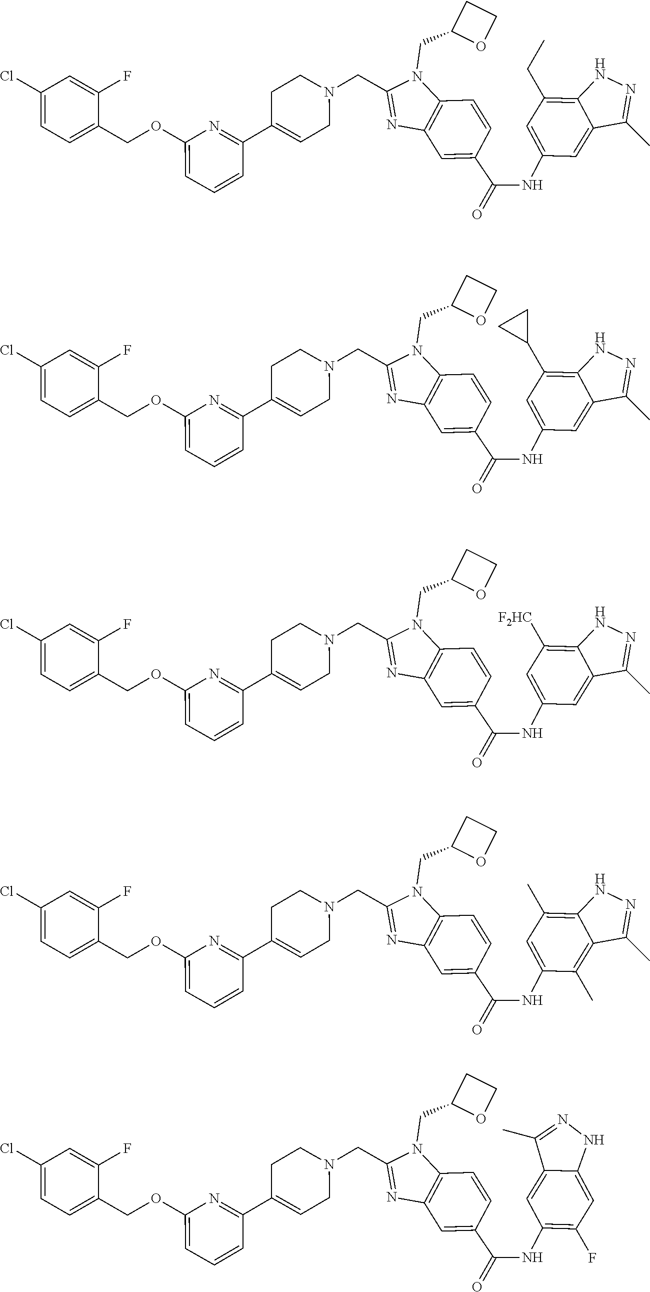

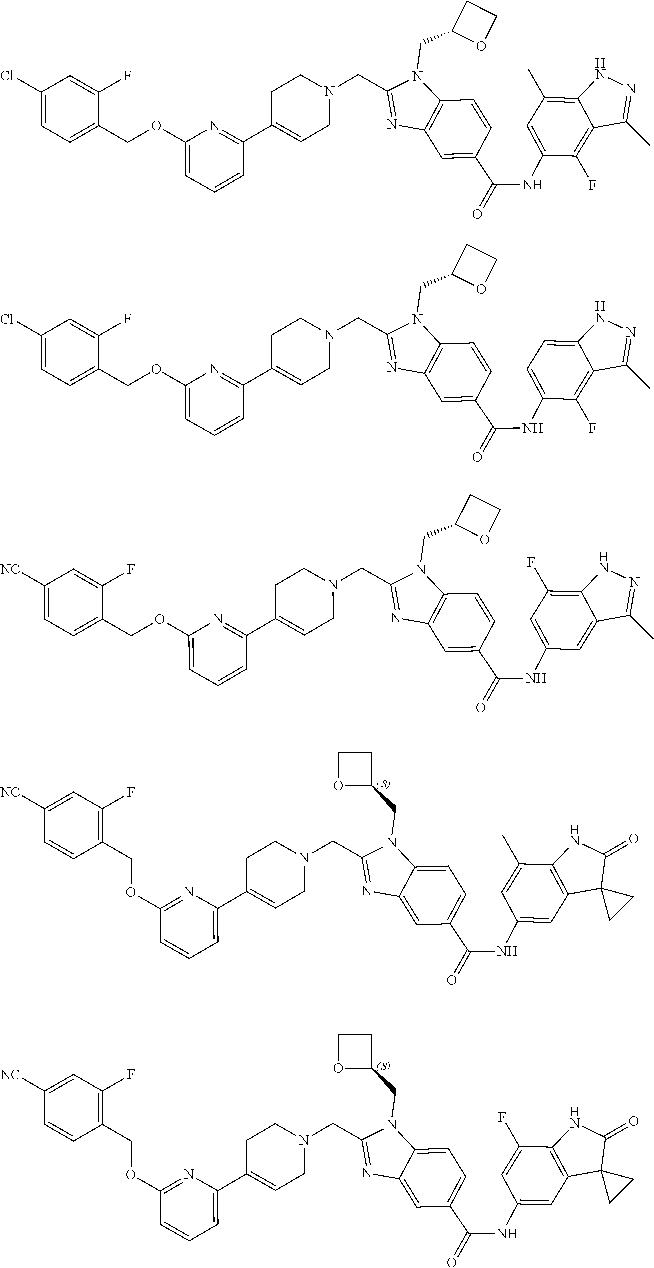

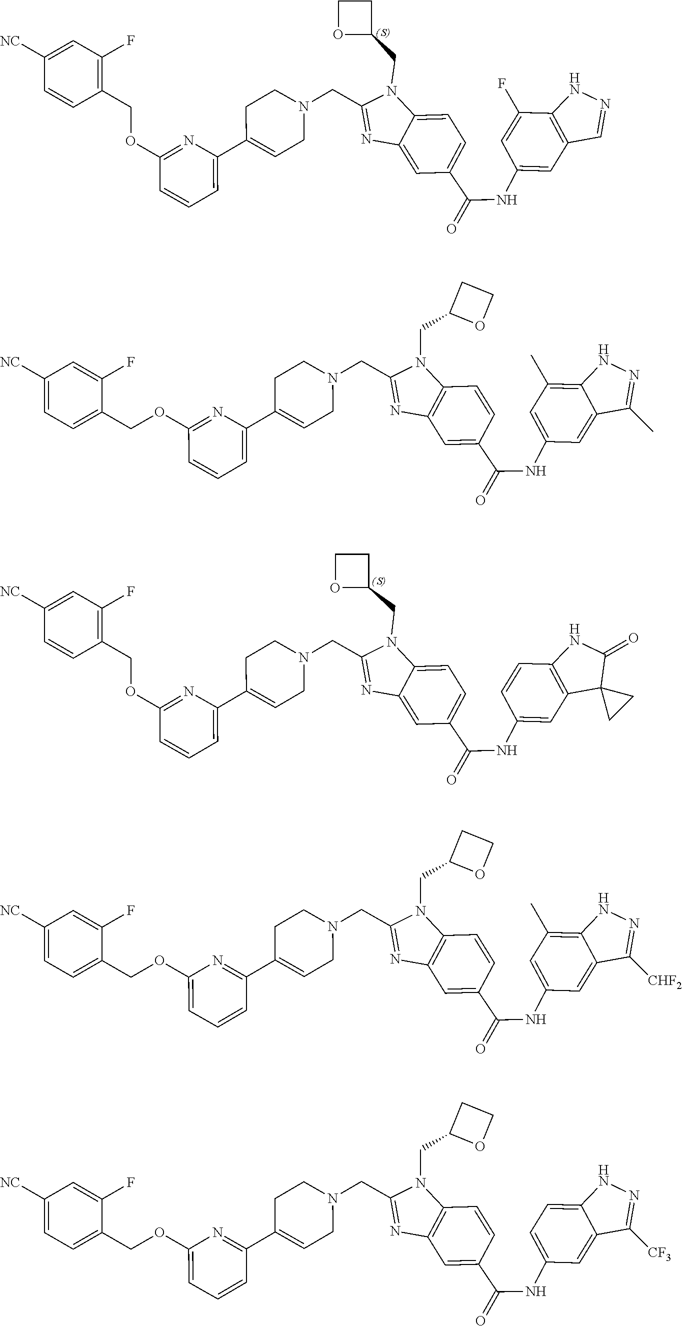

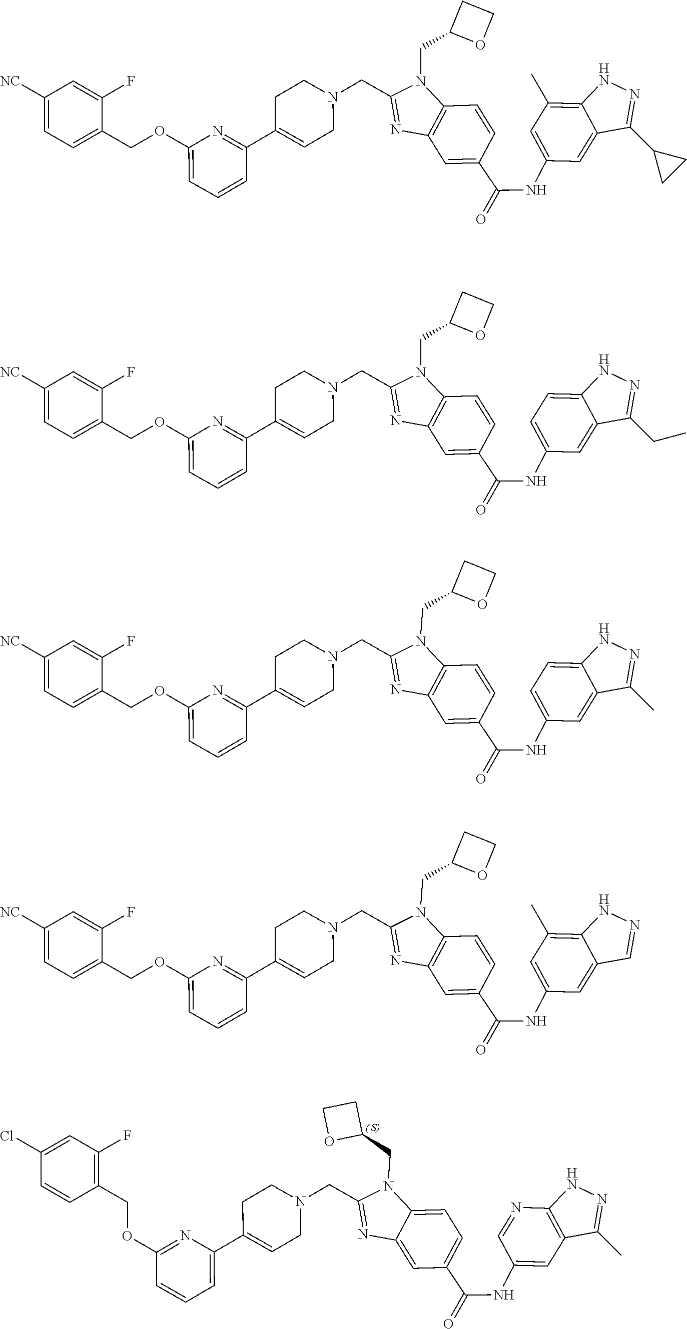

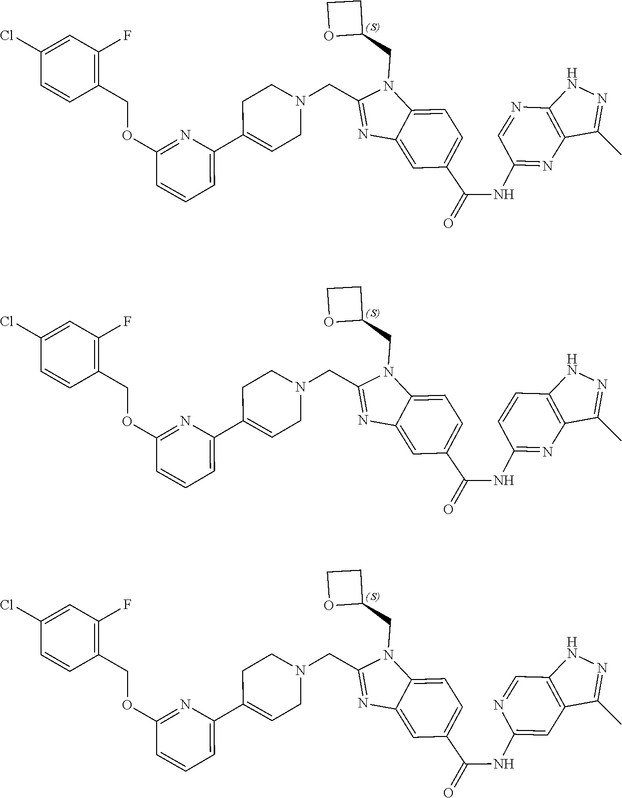

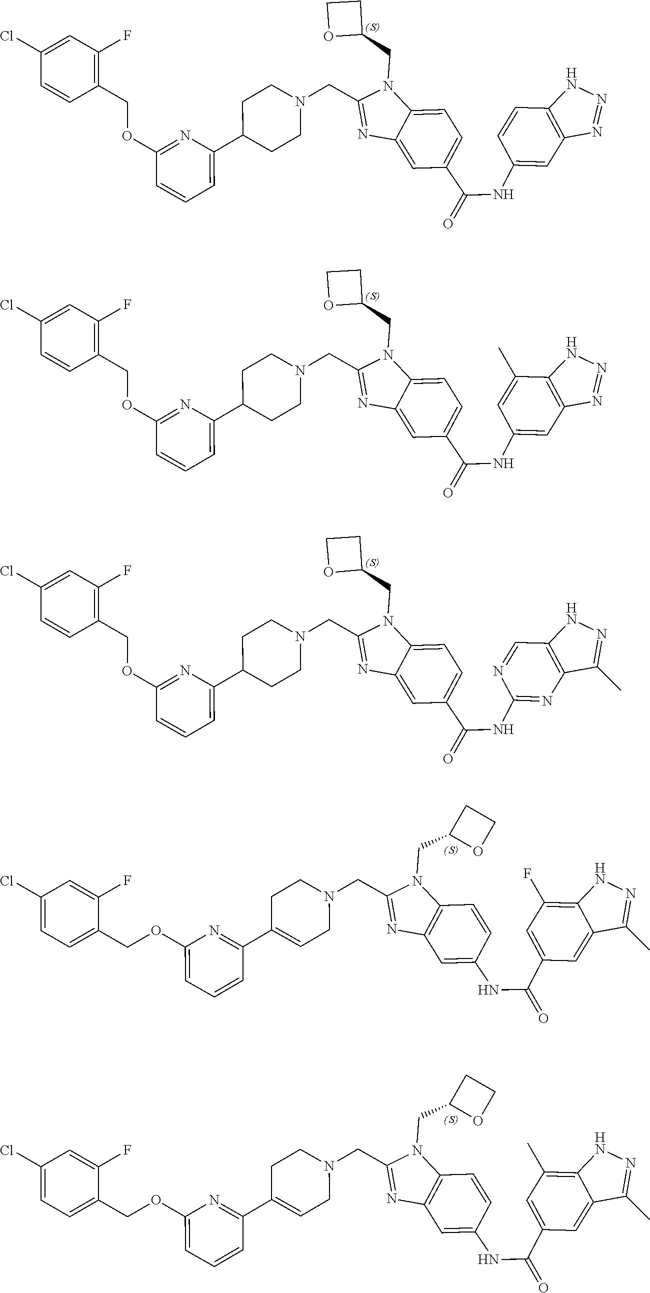

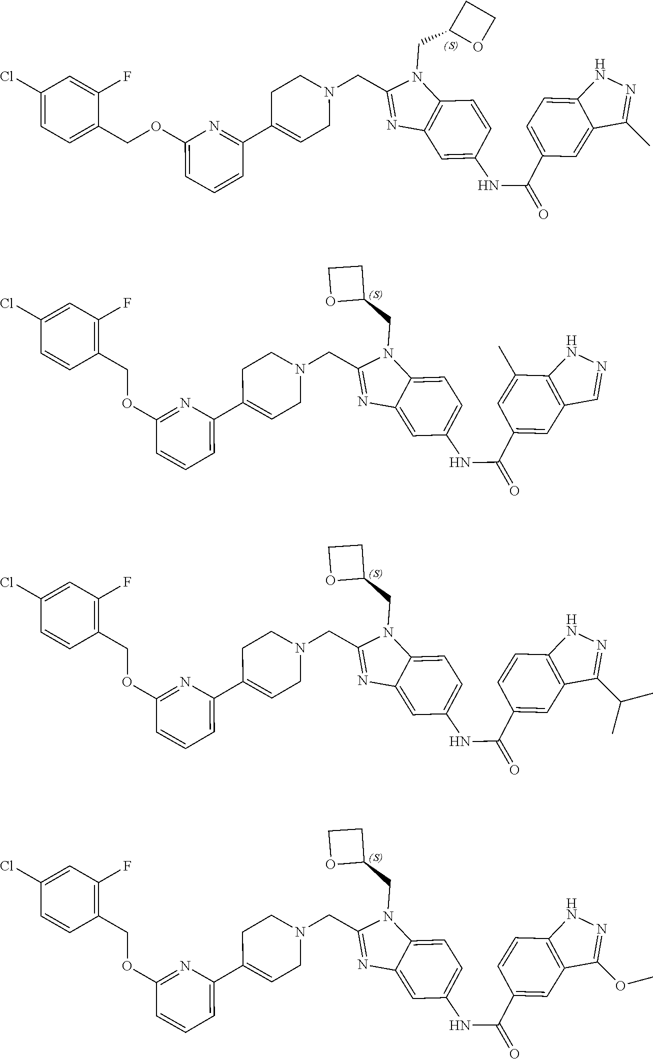

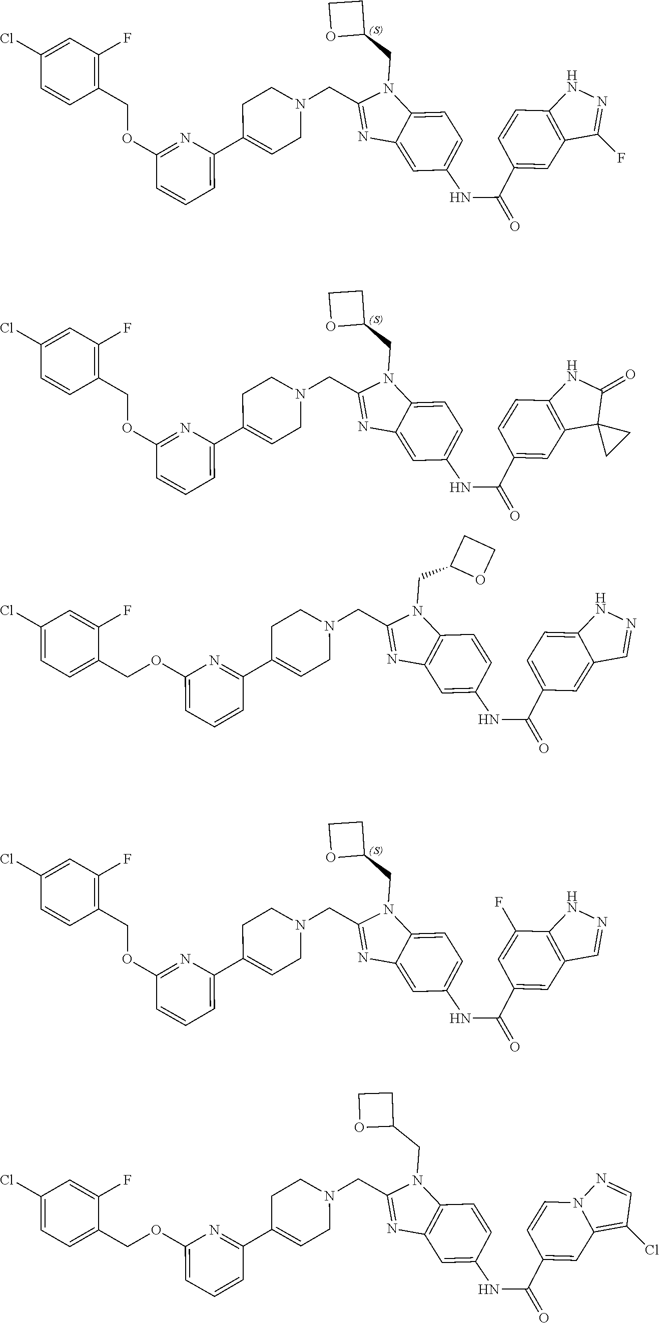

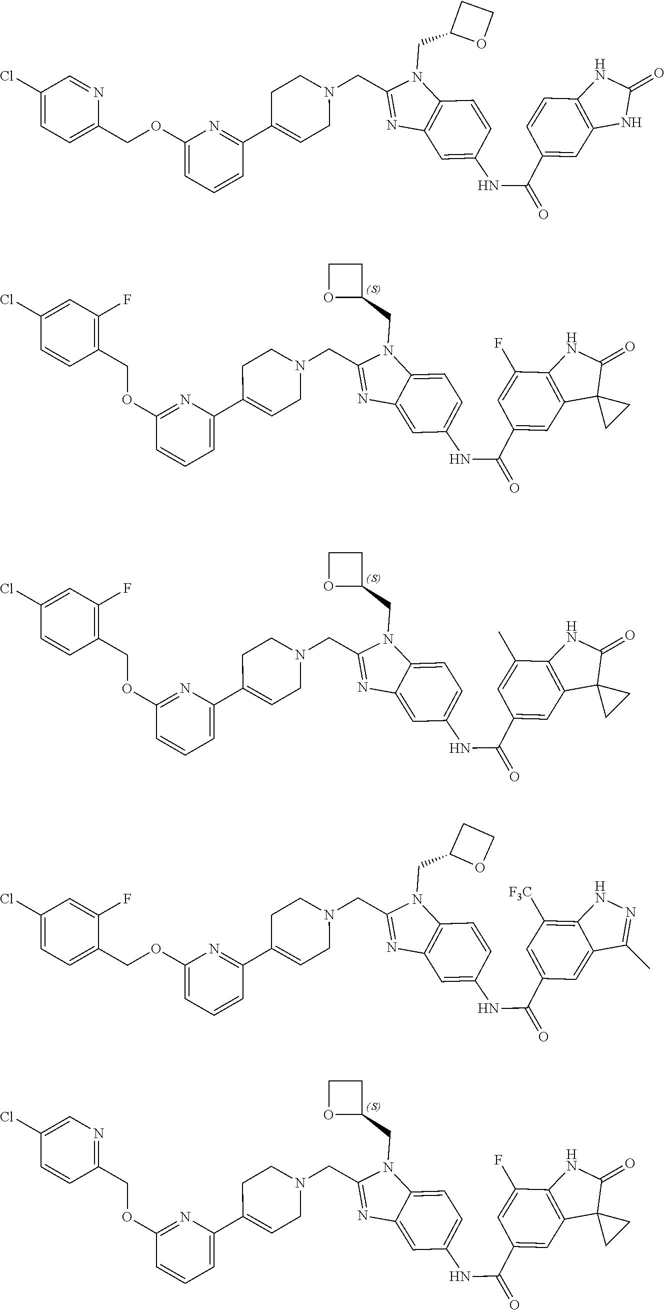

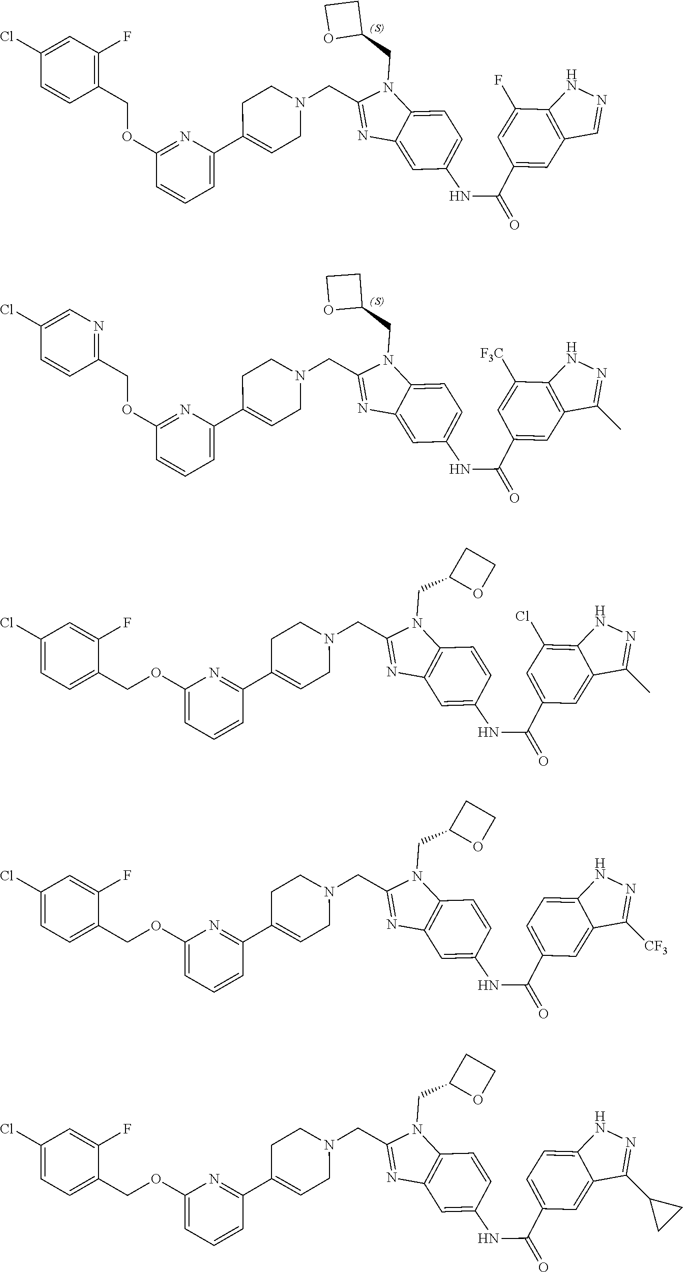

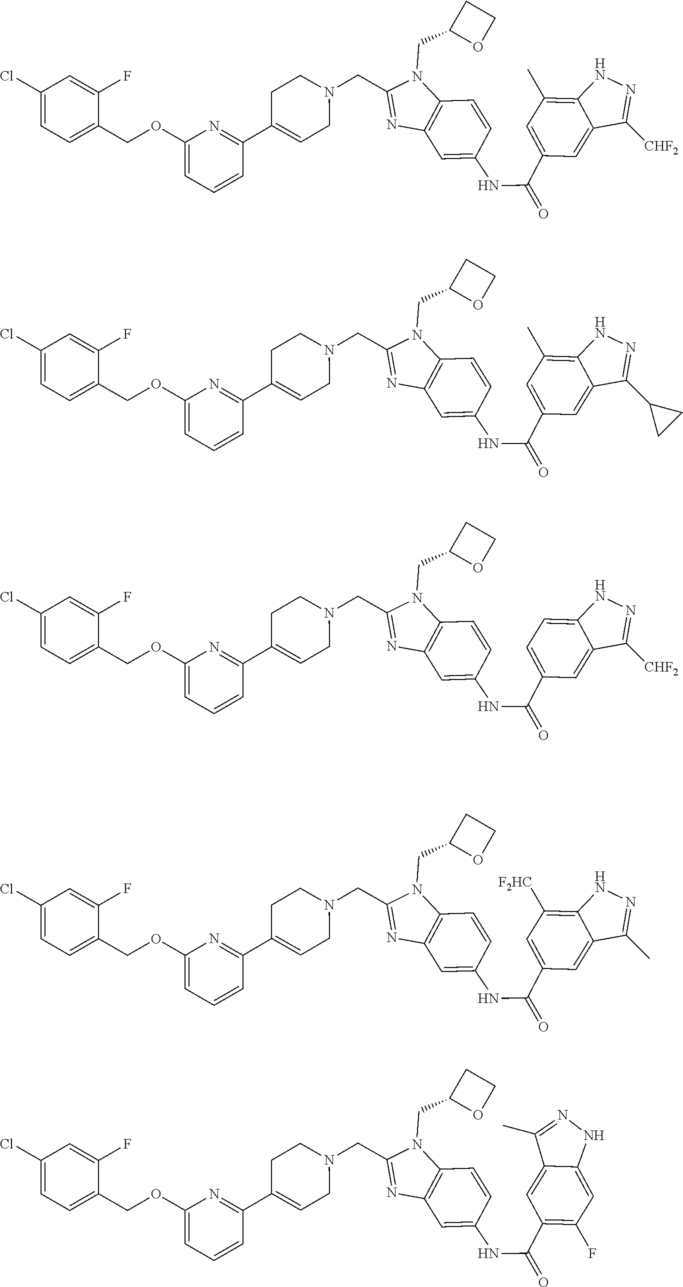 [SMILES: Cc1cc(C(=O)Nc2ccc3c(c2)nc(CN2CC=C(c4cccc(OCc5ccc(Cl)cc5F)n4)CC2)n3C[C@@H]2CCO2)cc2c(C(F)F)n[nH]c12 ; Cc1cc(C(=O)Nc2ccc3c(c2)nc(CN2CC=C(c4cccc(OCc5ccc(Cl)cc5F)n4)CC2)n3C[C@@H]2CCO2)cc2c(C3CC3)n[nH]c12 ; O=C(Nc1ccc2c(c1)nc(CN1CC=C(c3cccc(OCc4ccc(Cl)cc4F)n3)CC1)n2C[C@@H]1CCO1)c1ccc2[nH]nc(C(F)F)c2c1 ; Cc1n[nH]c2c(C(F)F)cc(C(=O)Nc3ccc4c(c3)nc(CN3CC=C(c5cccc(OCc6ccc(Cl)cc6F)n5)CC3)n4C[C@@H]3CCO3)cc12 ; Cc1n[nH]c2cc(F)c(C(=O)Nc3ccc4c(c3)nc(CN3CC=C(c5cccc(OCc6ccc(Cl)cc6F)n5)CC3)n4C[C@@H]3CCO3)cc12]

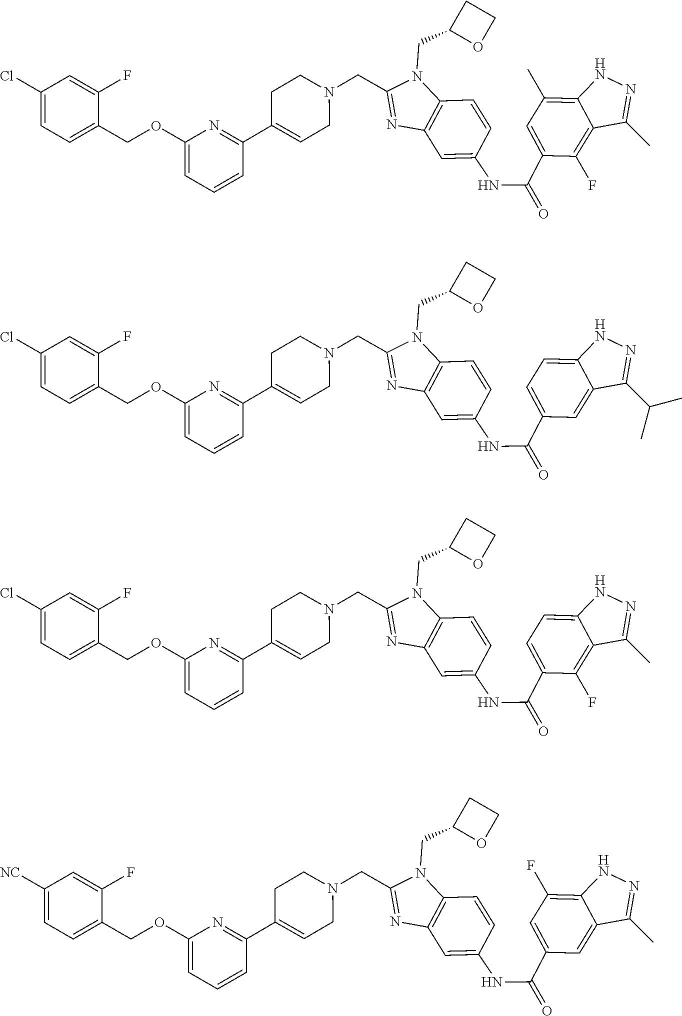 [SMILES: Cc1cc(C(=O)Nc2ccc3c(c2)nc(CN2CC=C(c4cccc(OCc5ccc(Cl)cc5F)n4)CC2)n3C[C@@H]2CCO2)c(F)c2c(C)n[nH]c12 ; CC(C)c1n[nH]c2ccc(C(=O)Nc3ccc4c(c3)nc(CN3CC=C(c5cccc(OCc6ccc(Cl)cc6F)n5)CC3)n4C[C@@H]3CCO3)cc12 ; Cc1n[nH]c2ccc(C(=O)Nc3ccc4c(c3)nc(CN3CC=C(c5cccc(OCc6ccc(Cl)cc6F)n5)CC3)n4C[C@@H]3CCO3)c(F)c12 ; Cc1n[nH]c2c(F)cc(C(=O)Nc3ccc4c(c3)nc(CN3CC=C(c5cccc(OCc6ccc(C#N)cc6F)n5)CC3)n4C[C@@H]3CCO3)cc12]

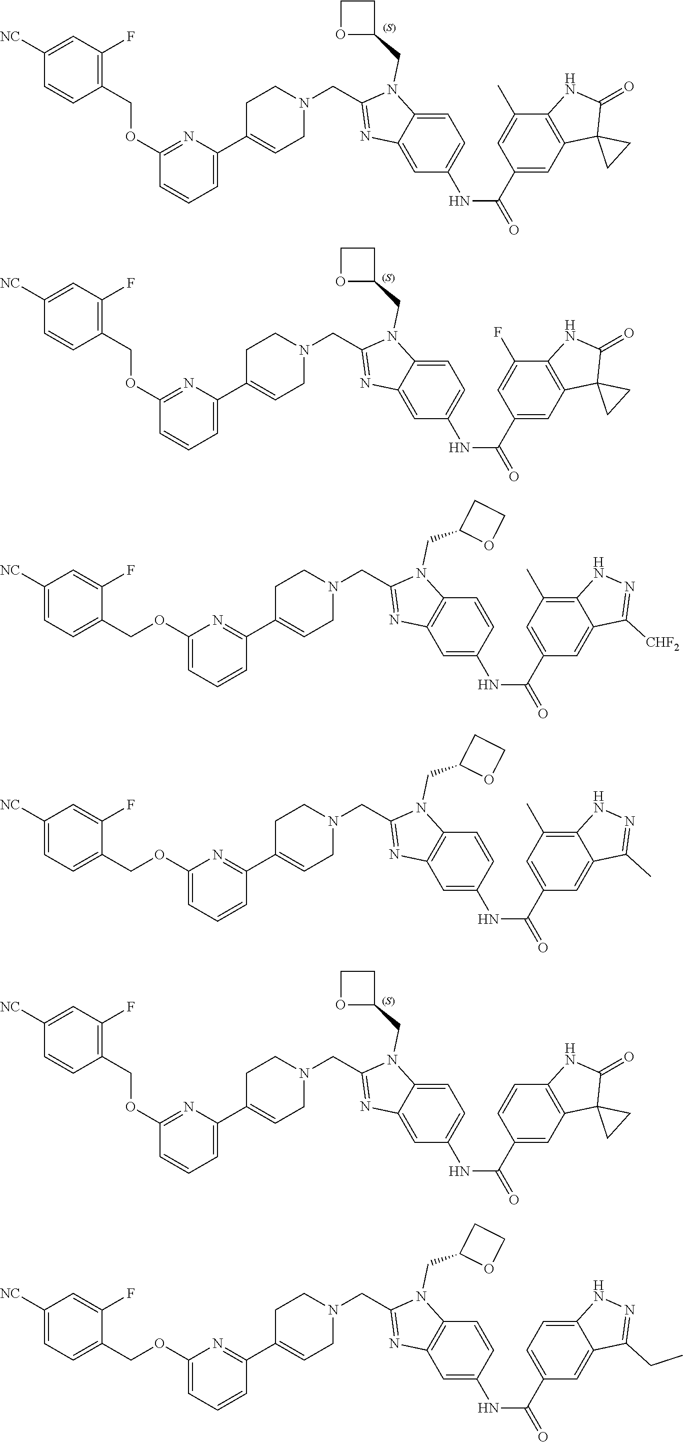

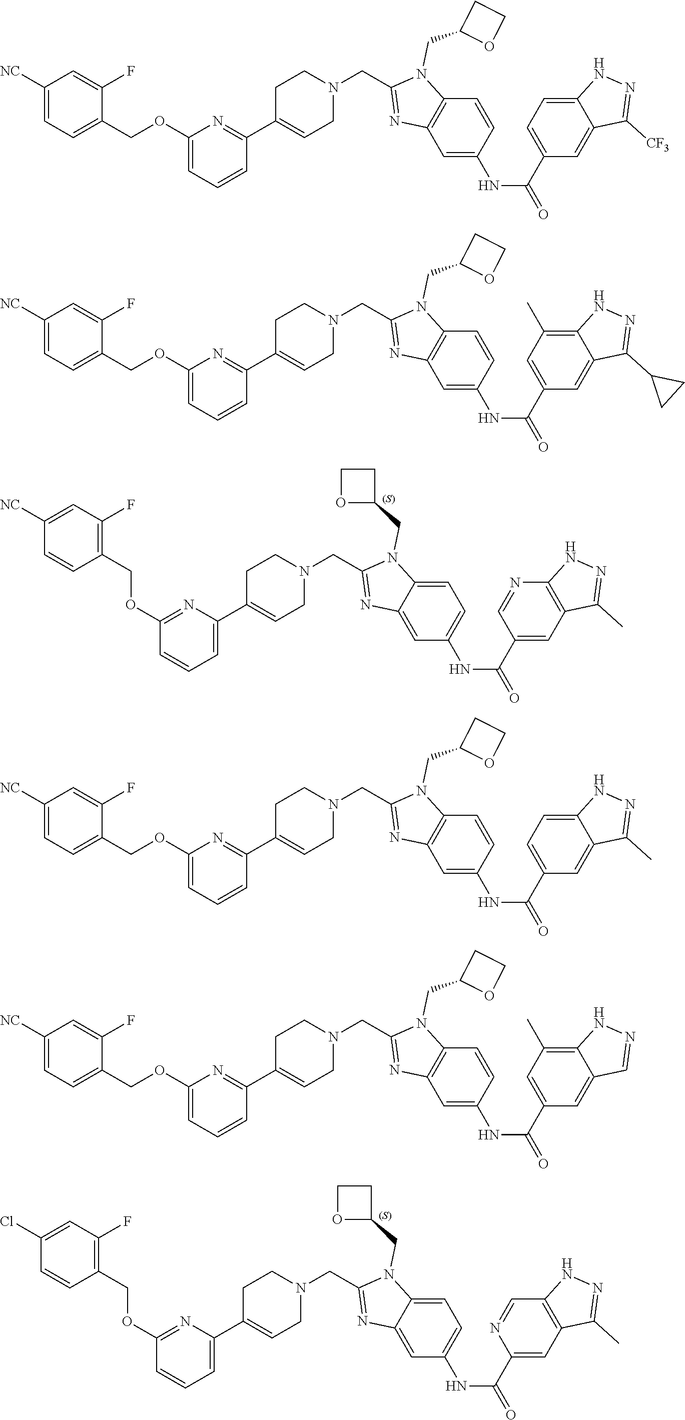

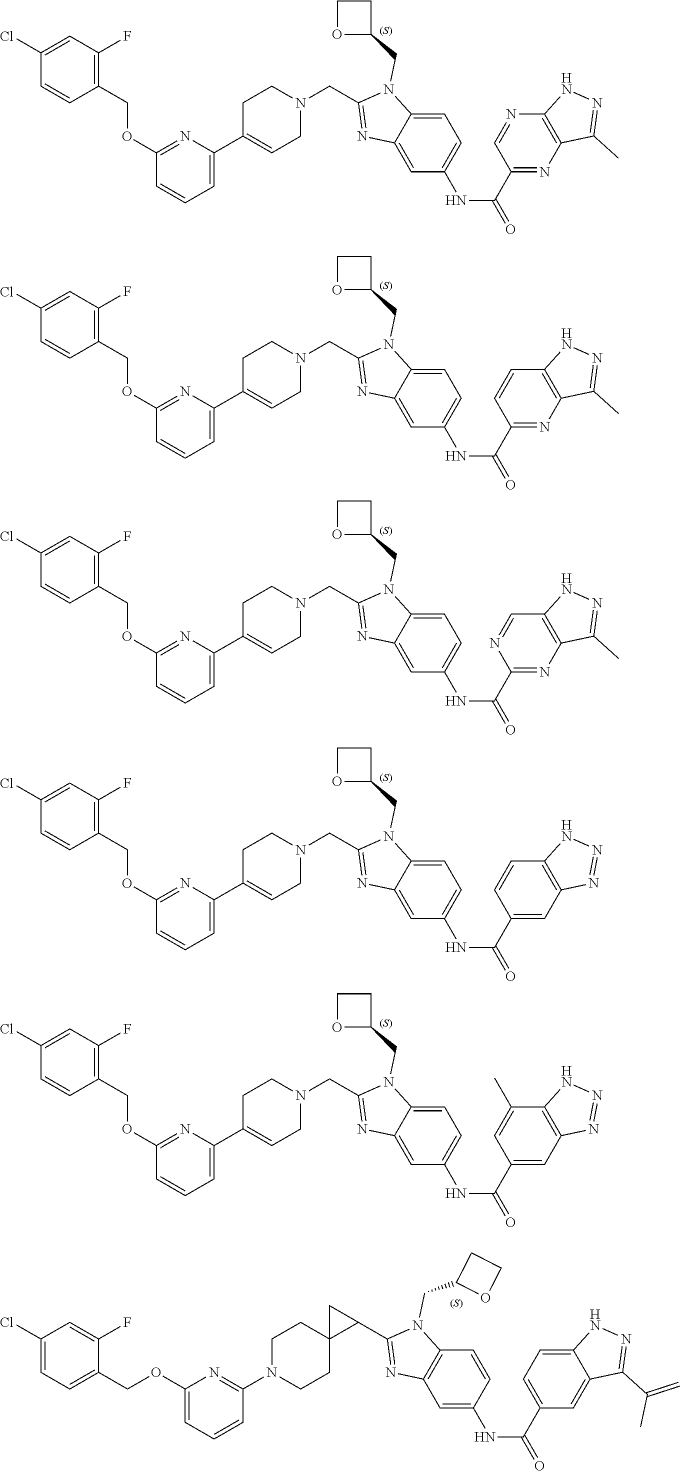

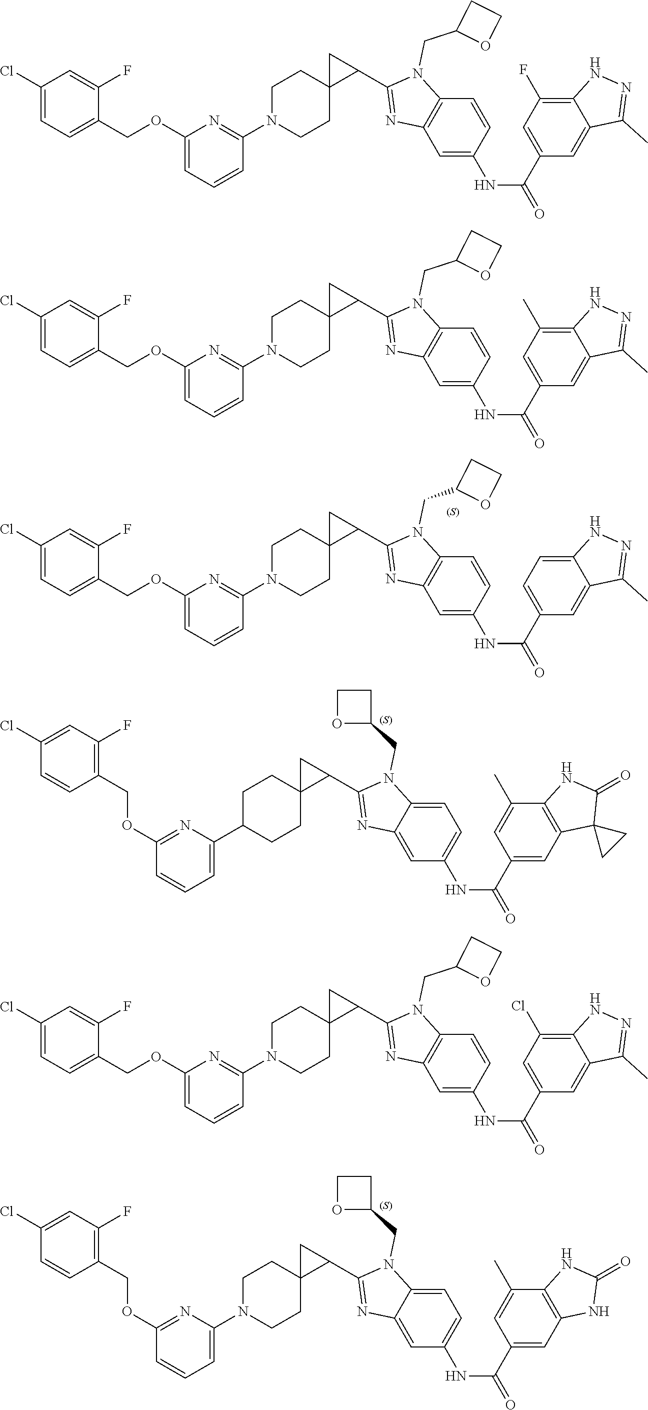

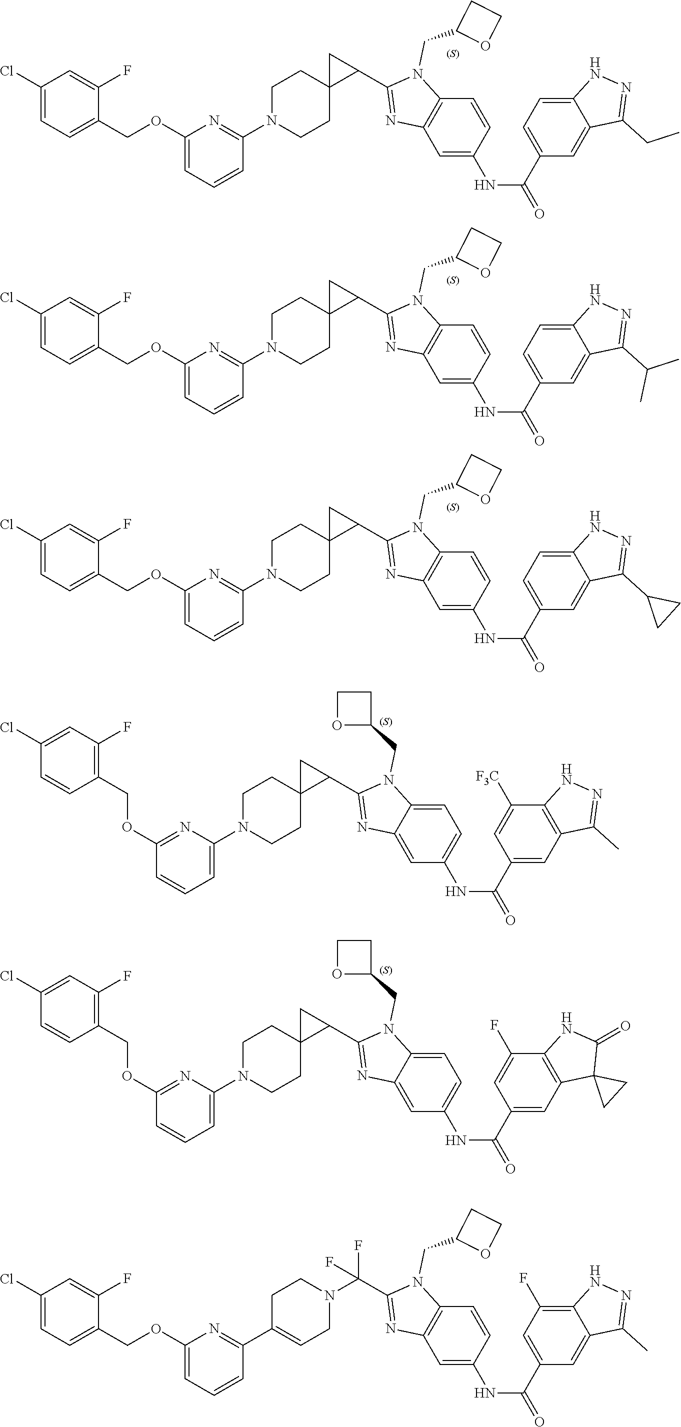

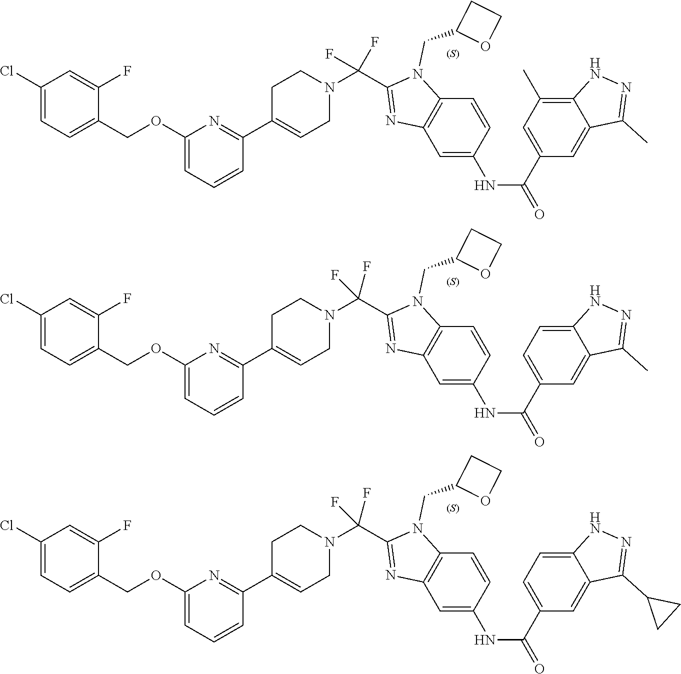

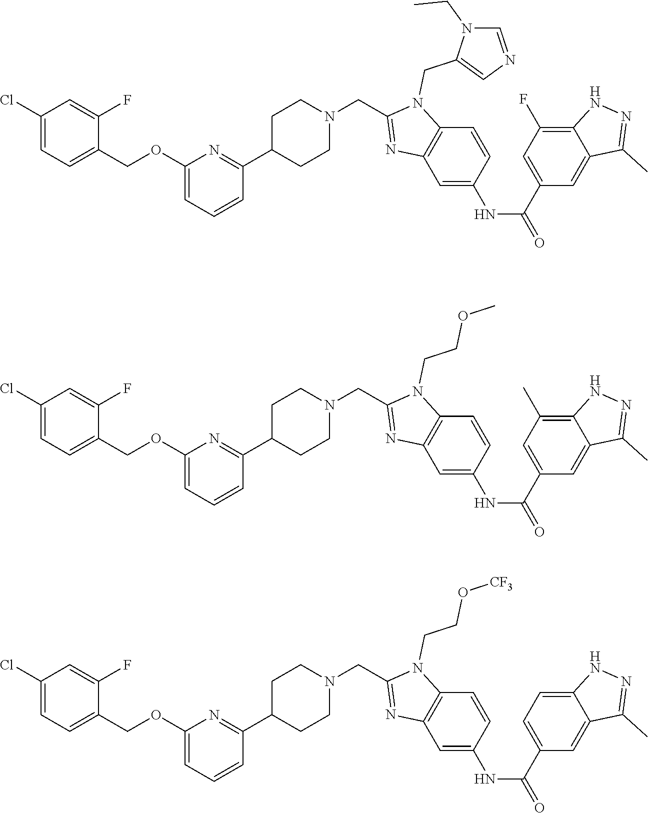 [SMILES: CCn1cncc1Cn1c(CN2CCC(c3cccc(OCc4ccc(Cl)cc4F)n3)CC2)nc2cc(NC(=O)c3cc(F)c4[nH]nc(C)c4c3)ccc21 ; COCCn1c(CN2CCC(c3cccc(OCc4ccc(Cl)cc4F)n3)CC2)nc2cc(NC(=O)c3cc(C)c4[nH]nc(C)c4c3)ccc21 ; Cc1n[nH]c2ccc(C(=O)Nc3ccc4c(c3)nc(CN3CCC(c5cccc(OCc6ccc(Cl)cc6F)n5)CC3)n4CCOC(F)(F)F)cc12]

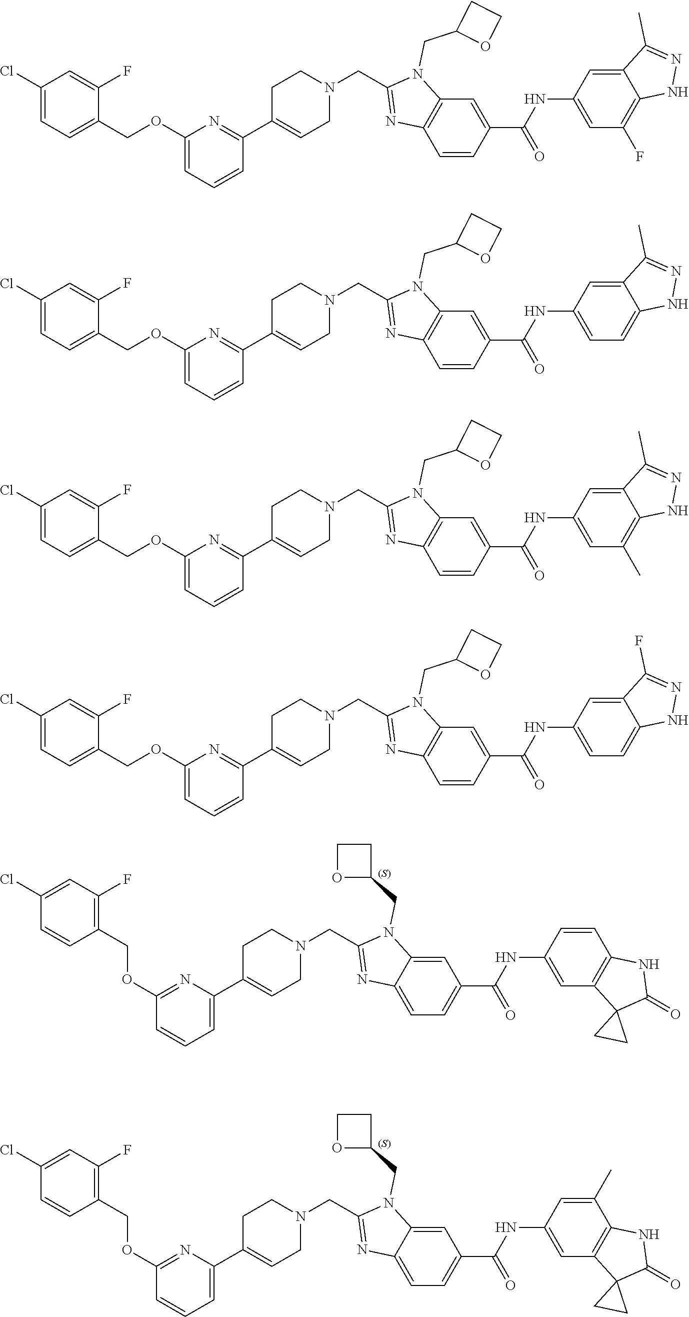

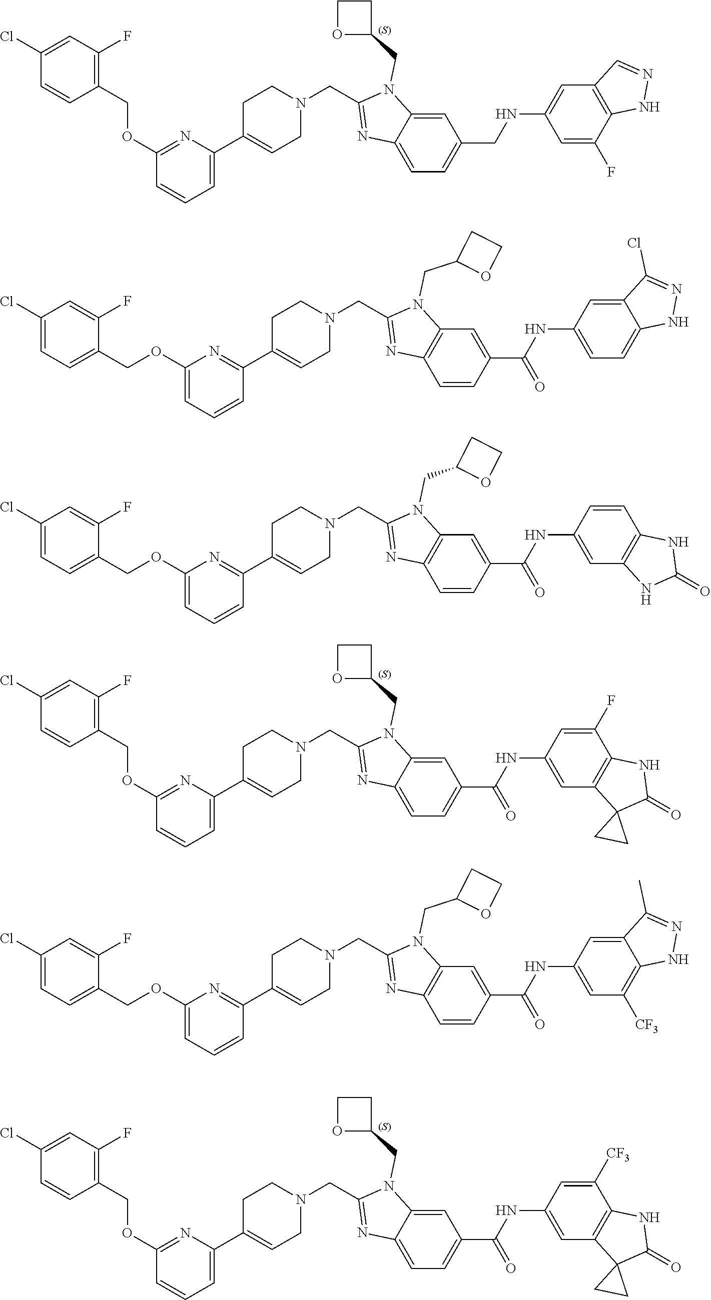

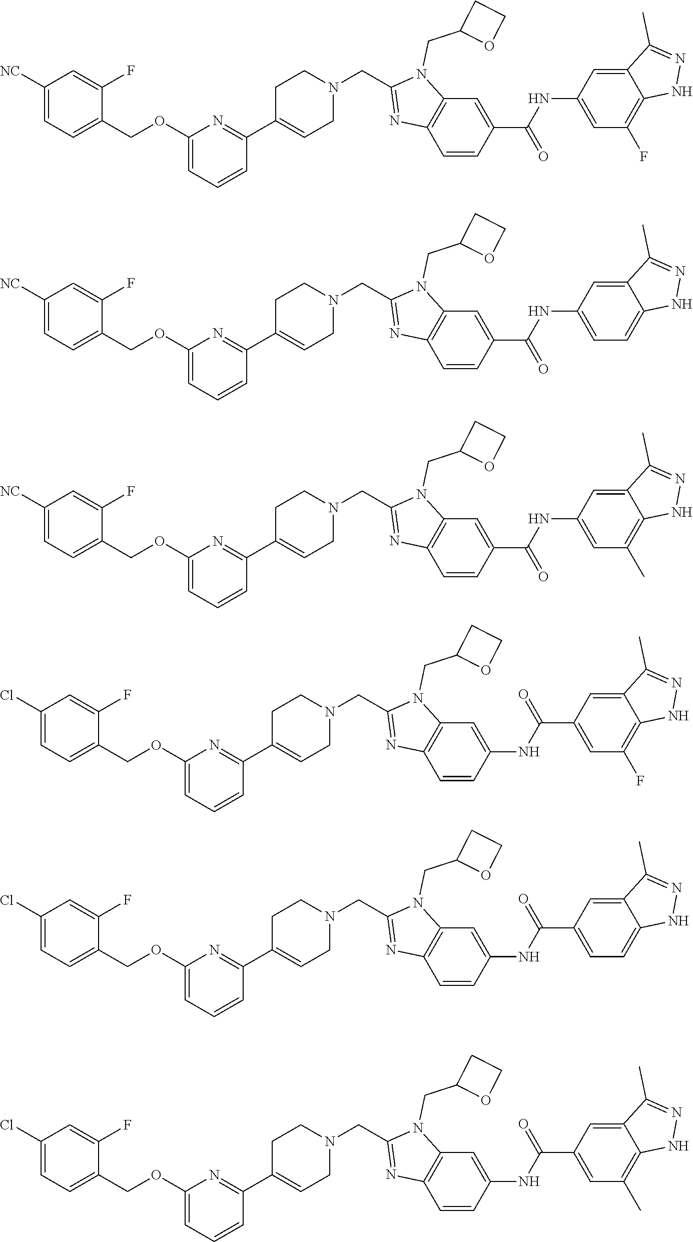

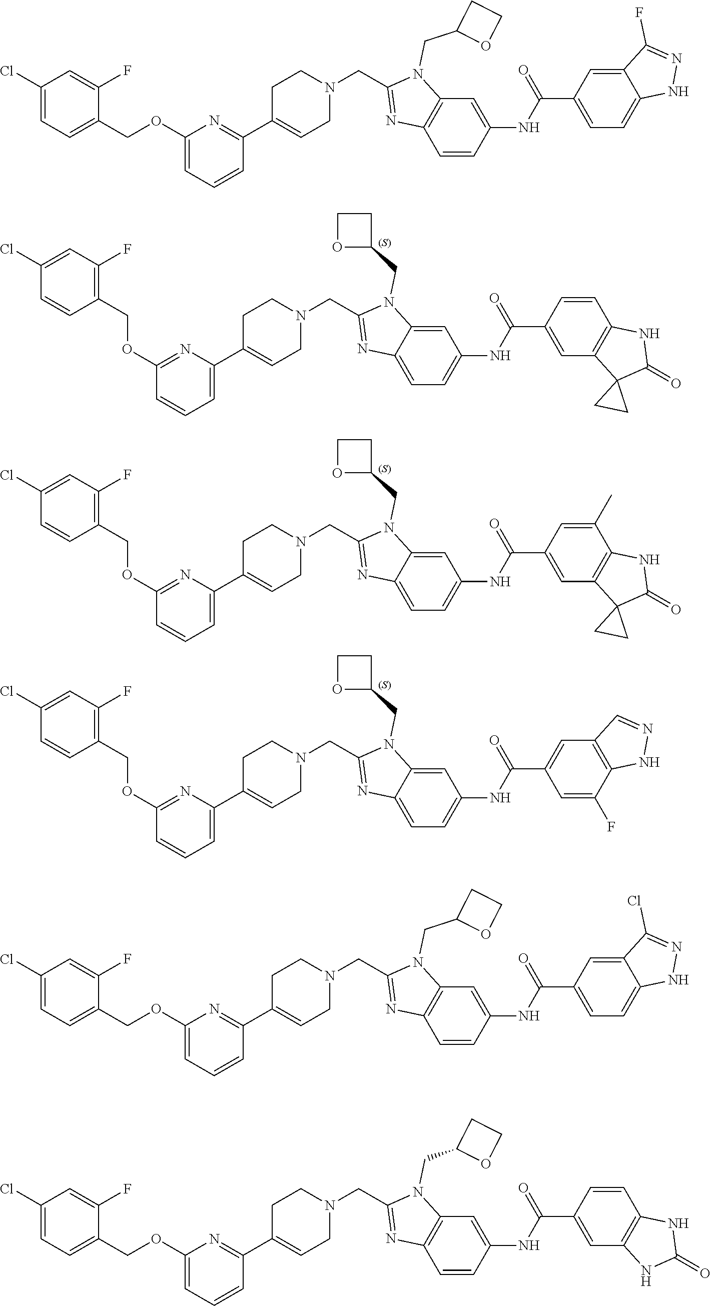

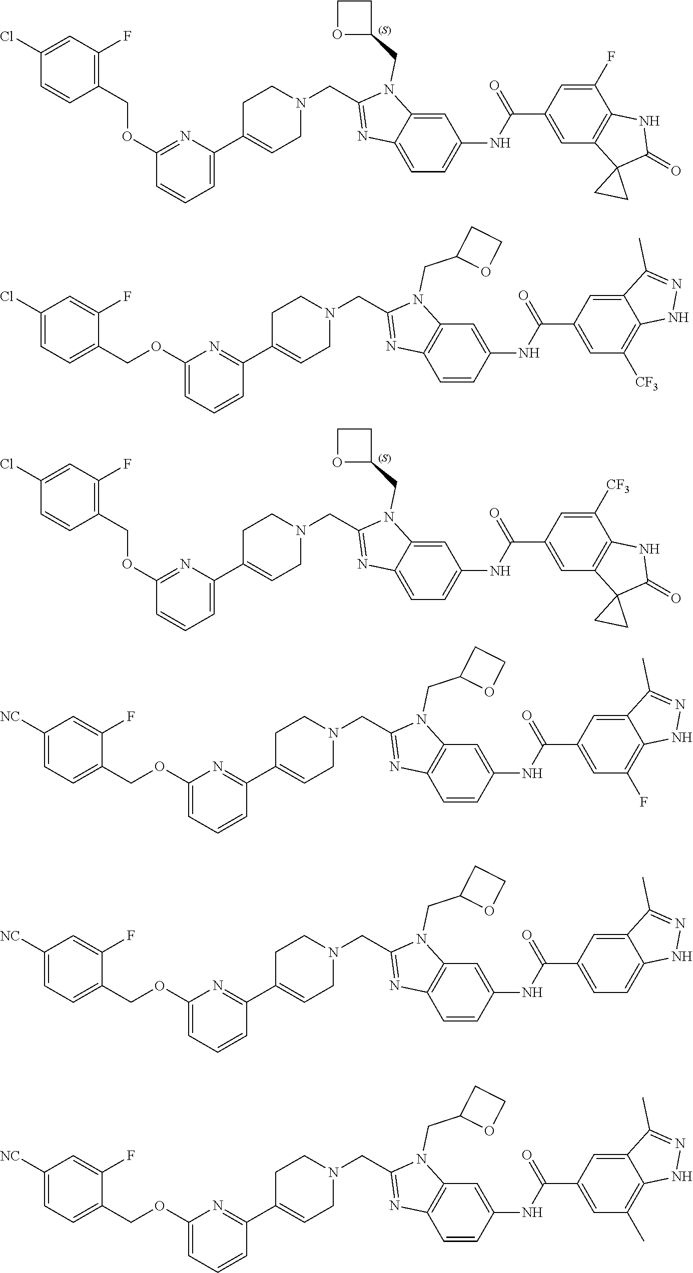

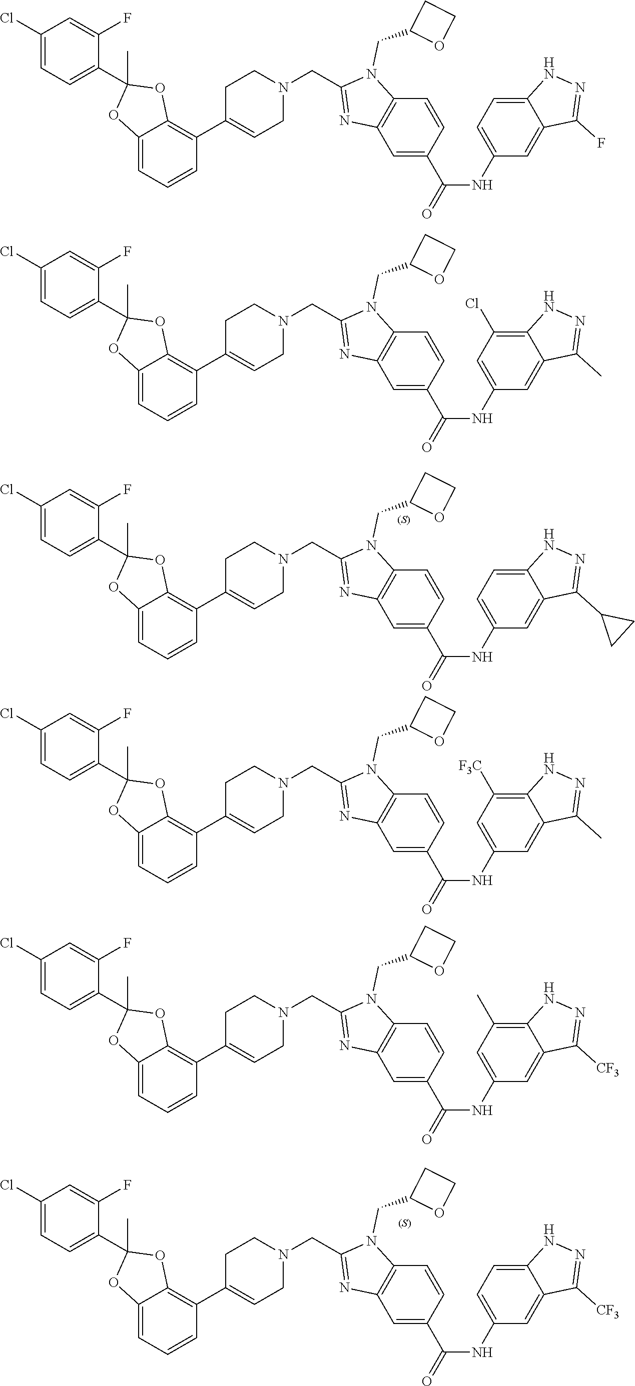

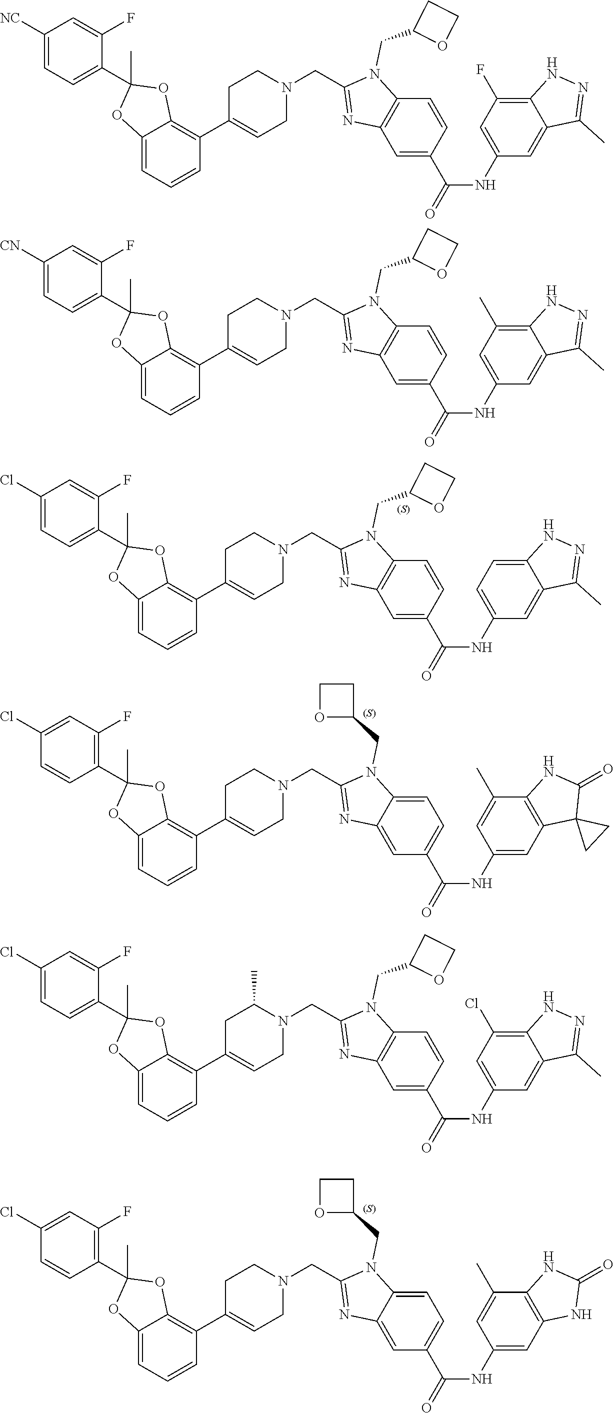

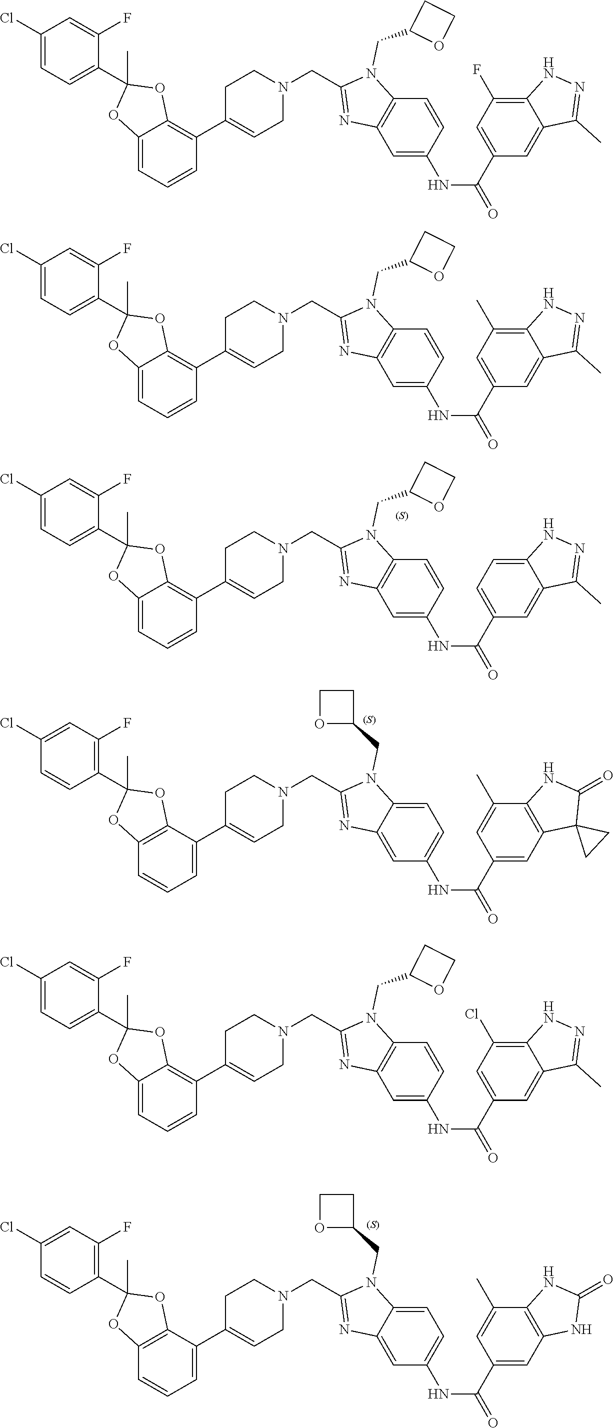

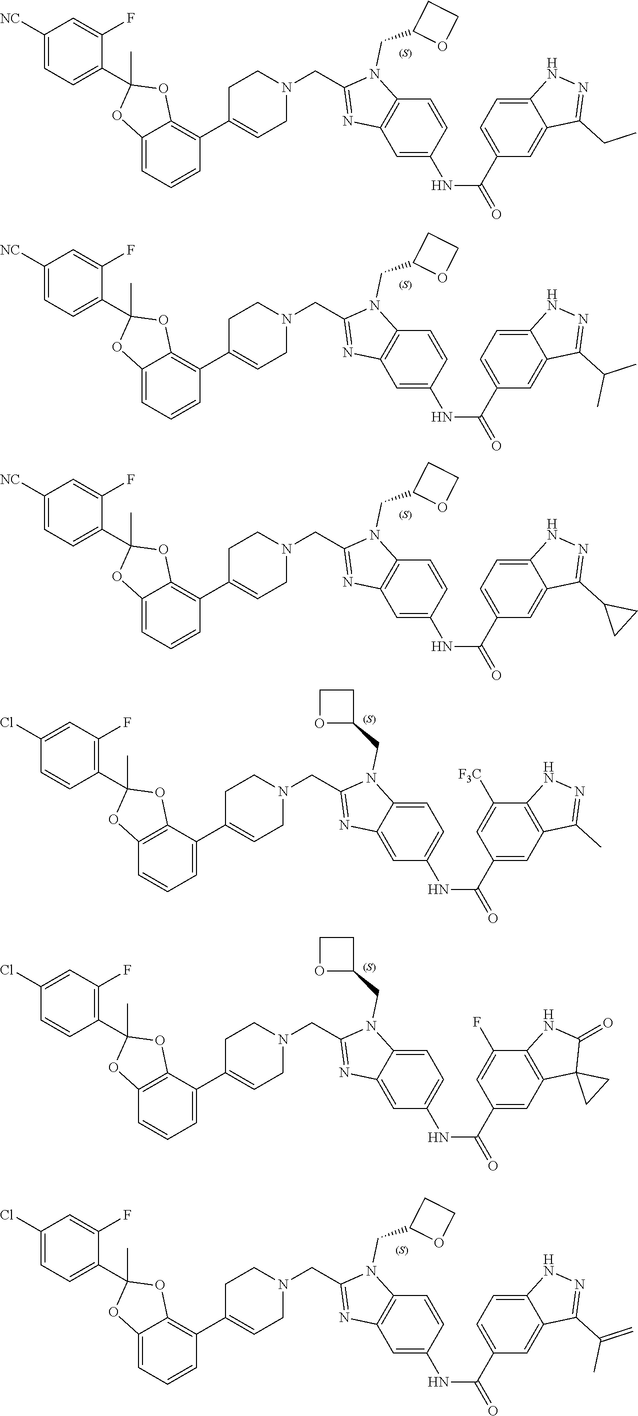

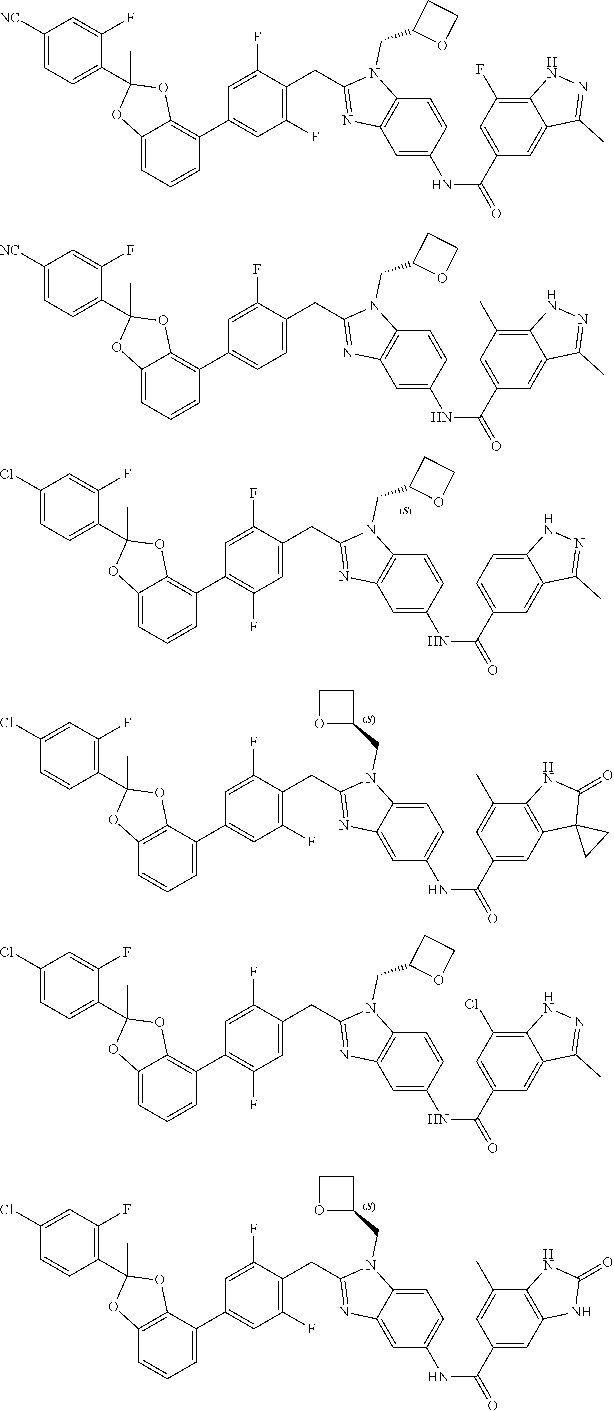

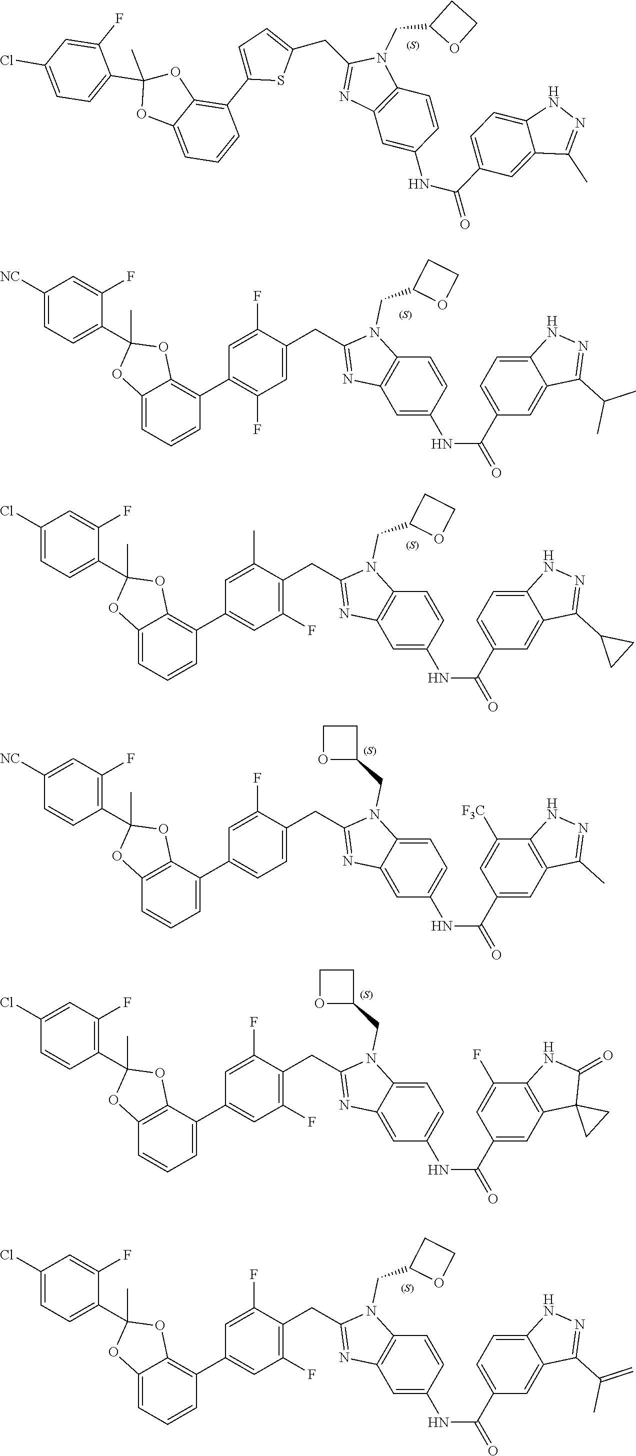

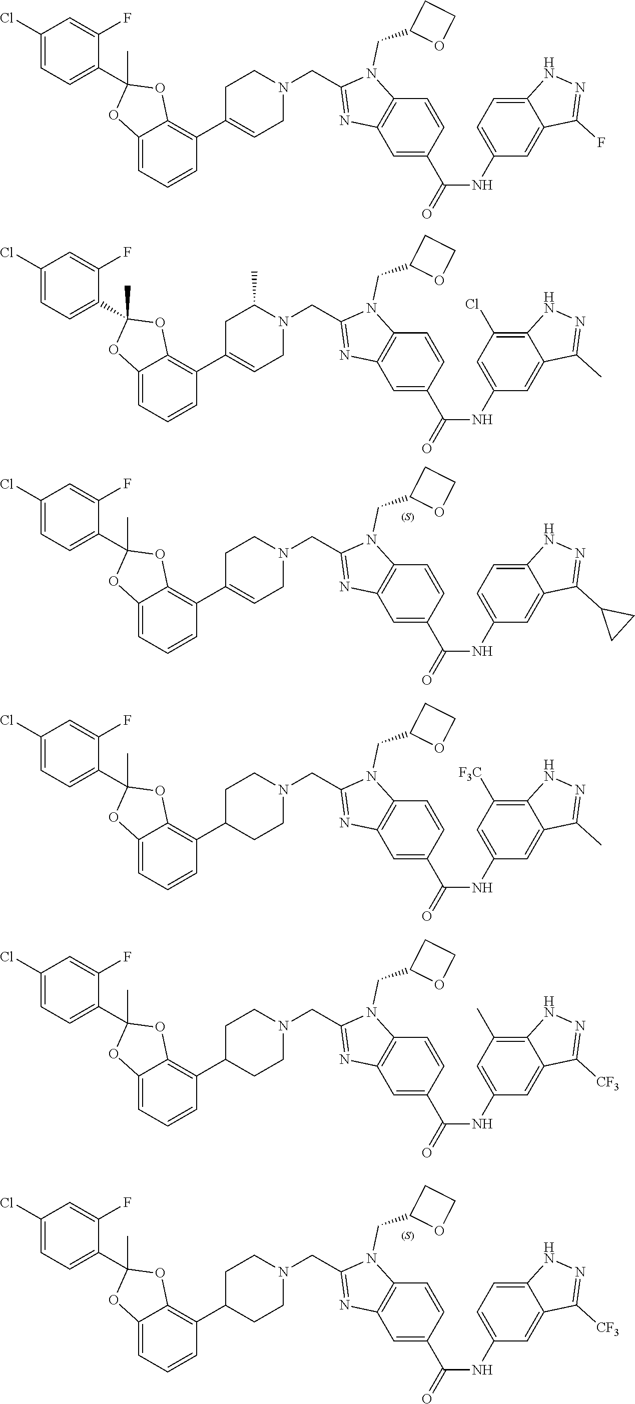

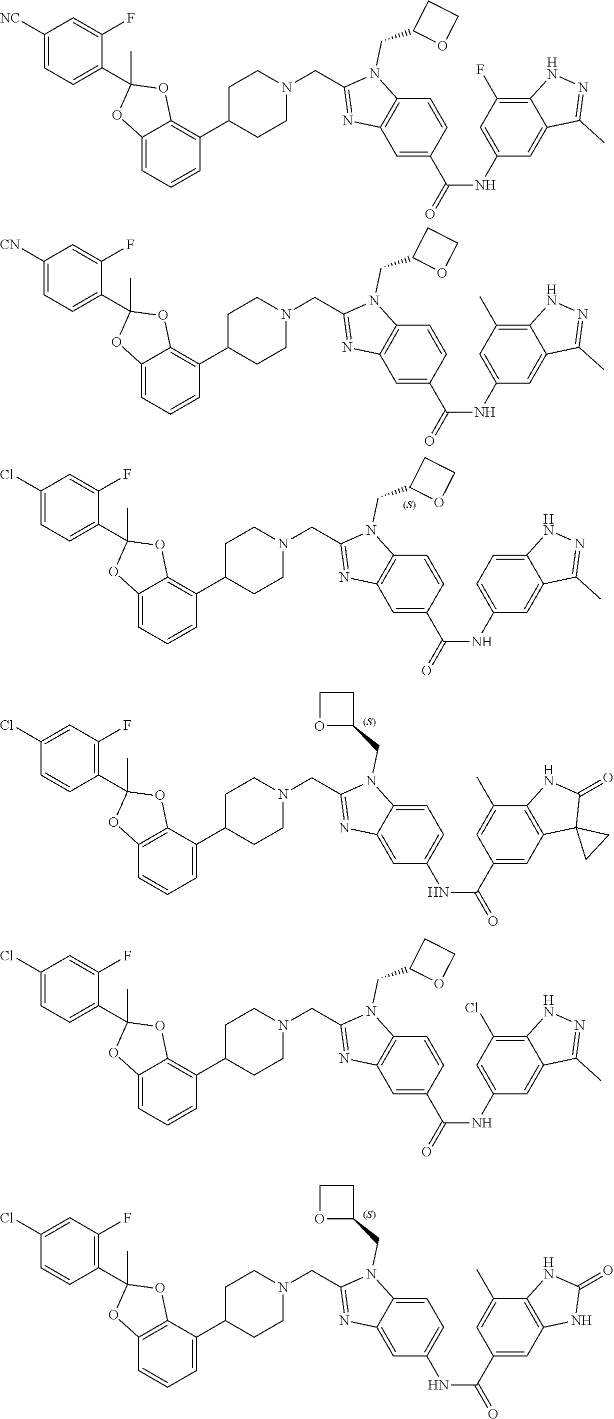

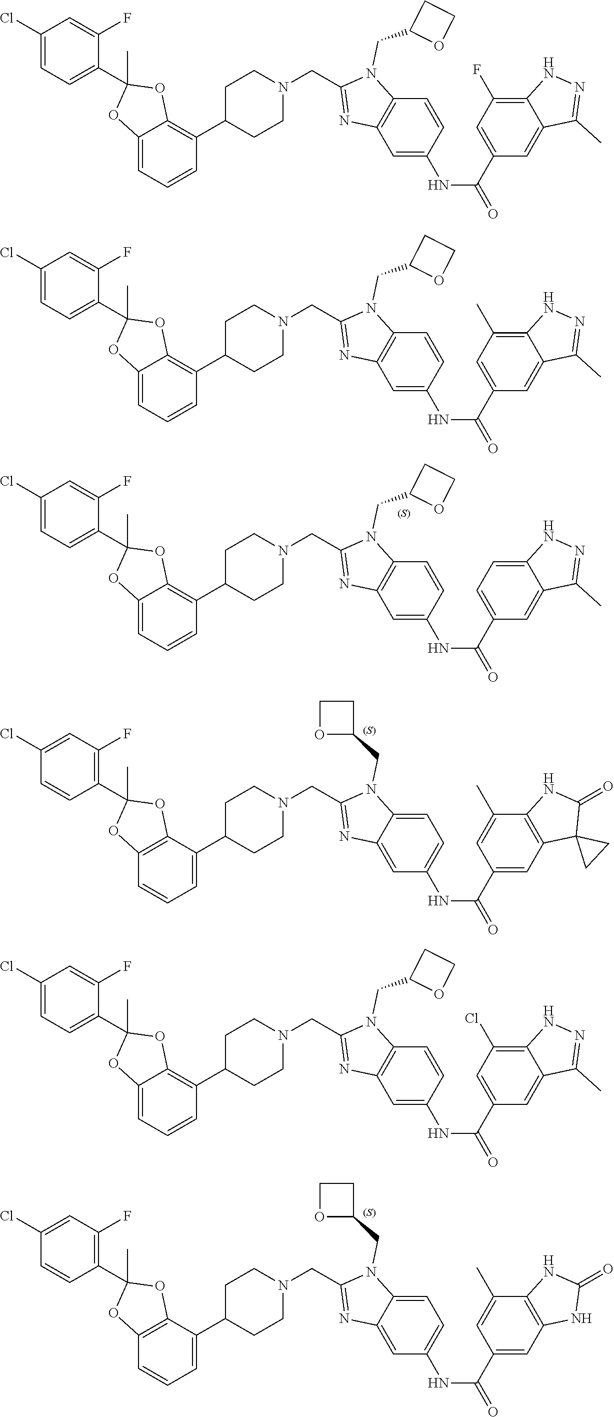

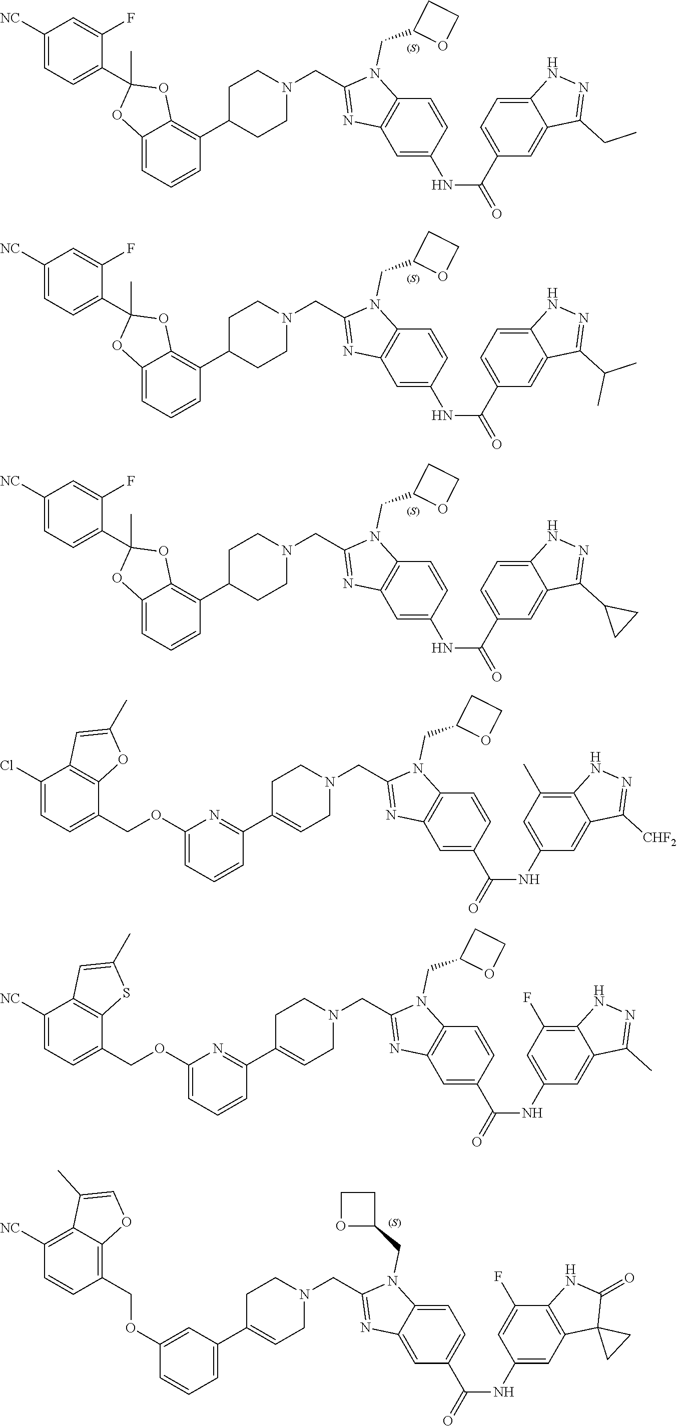

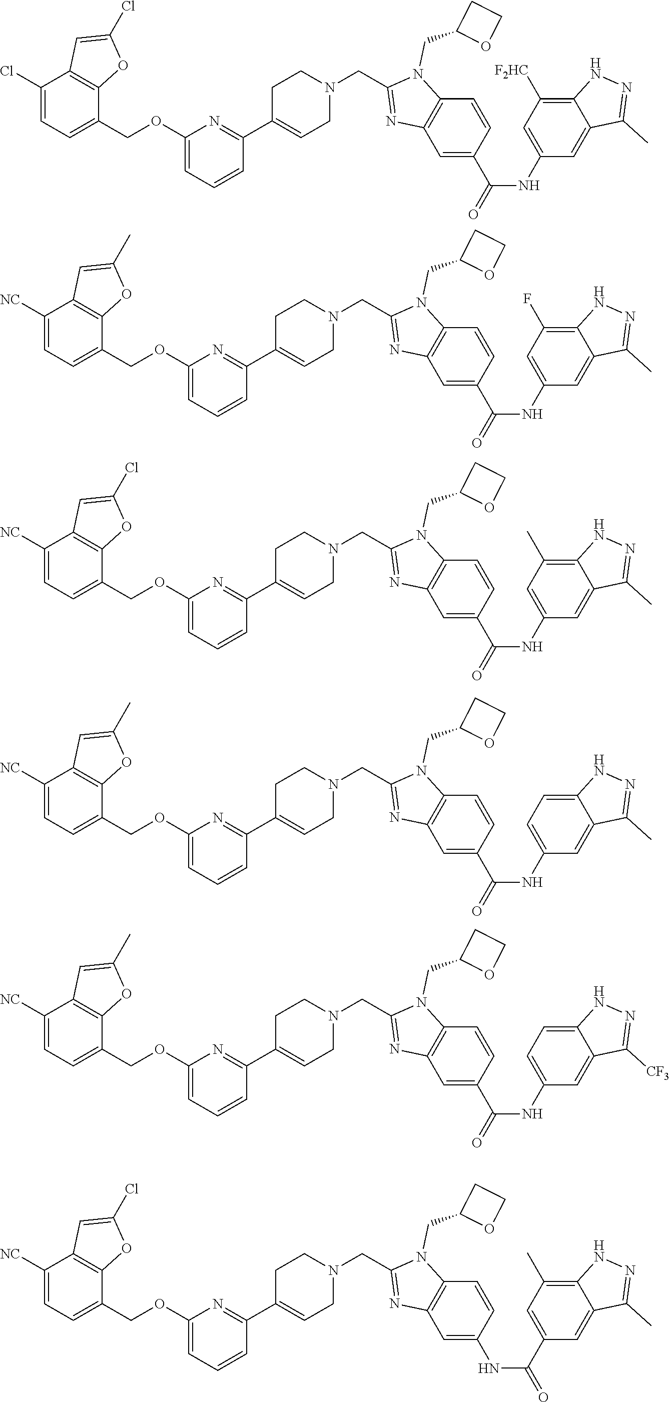

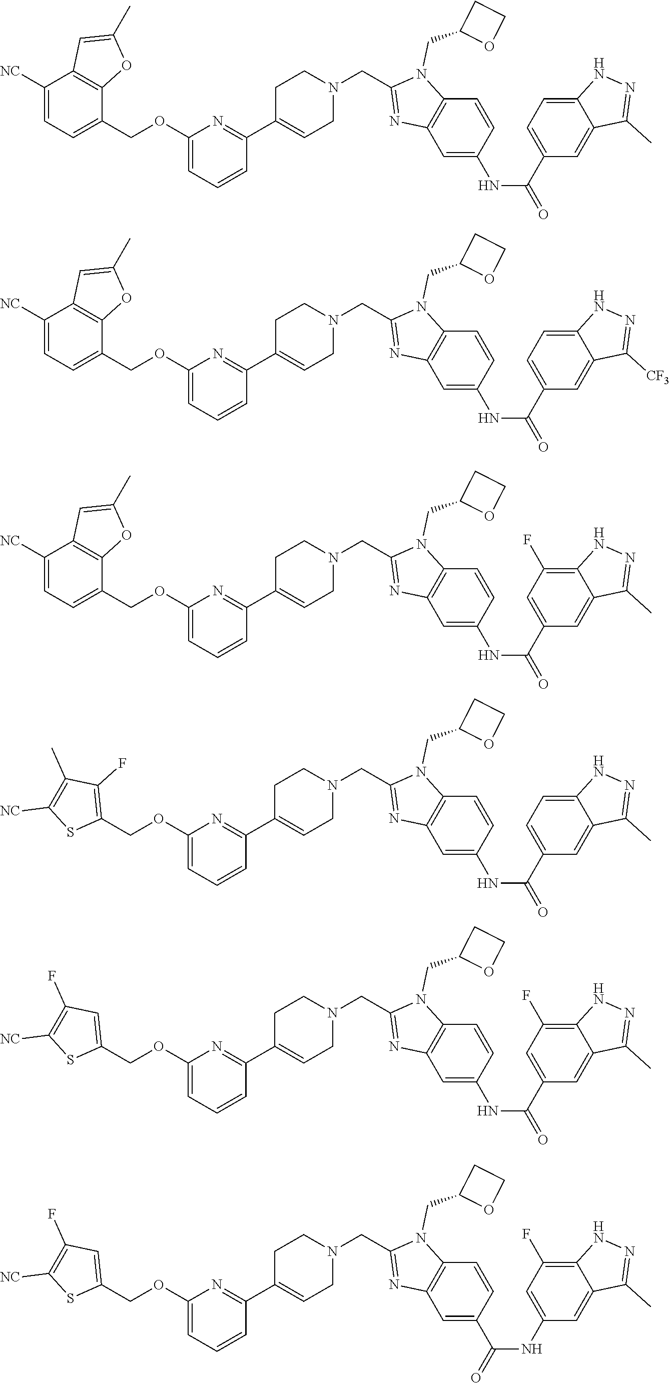

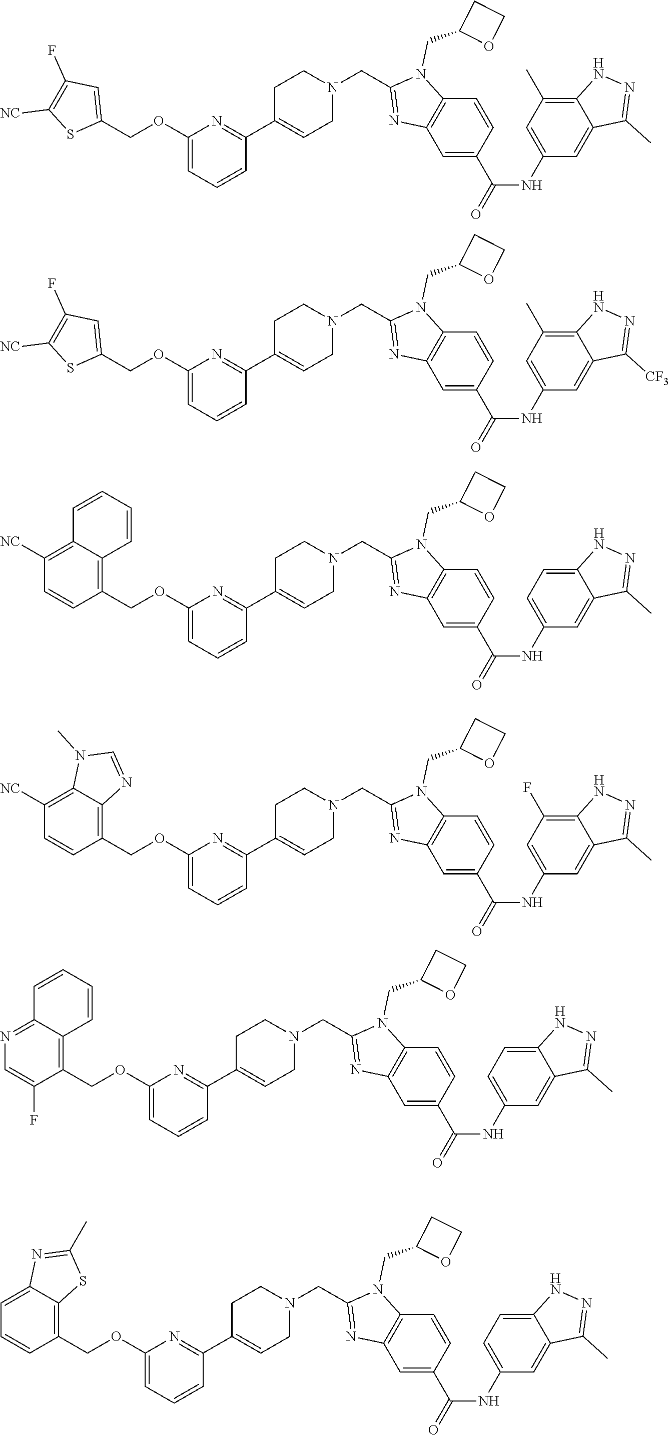

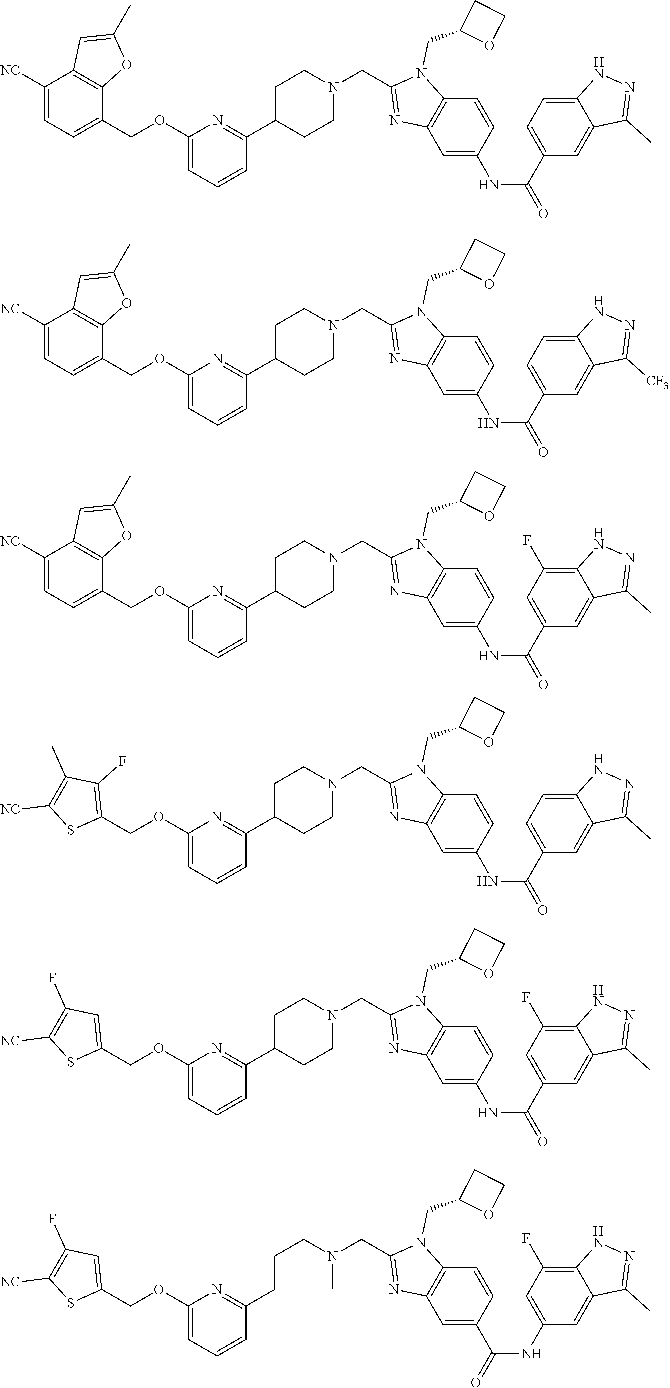

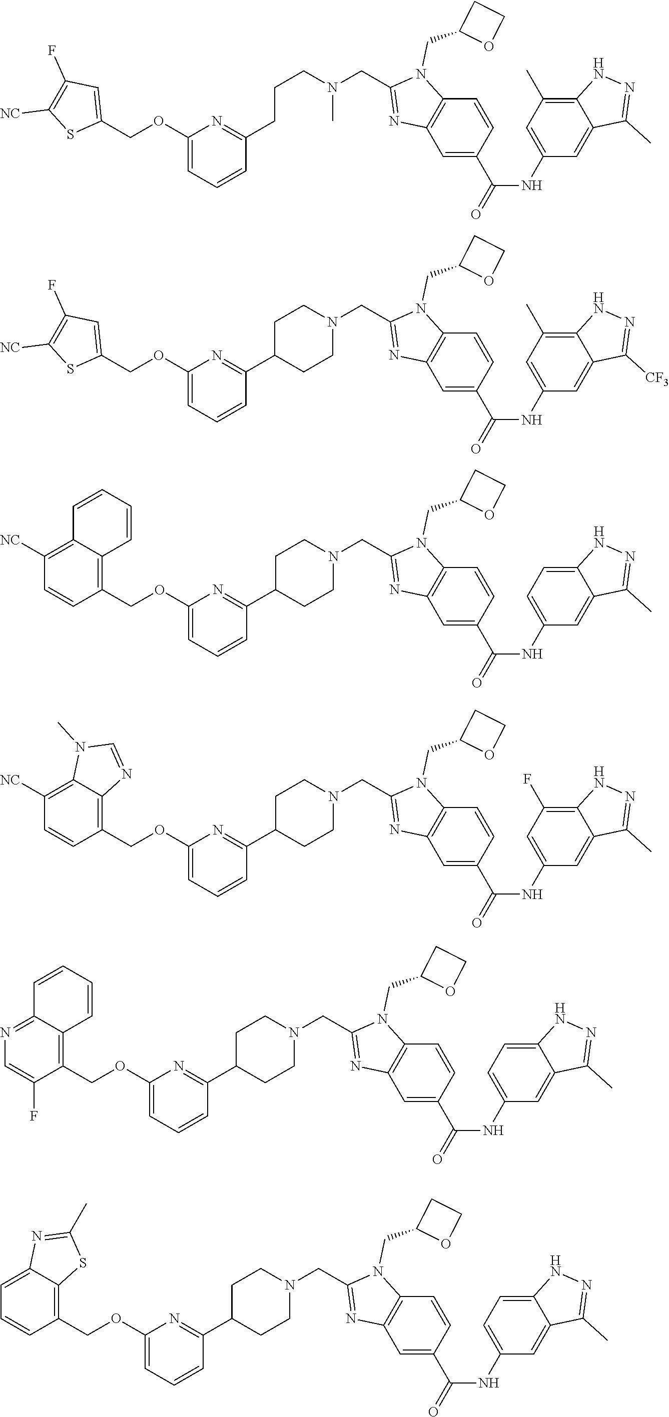

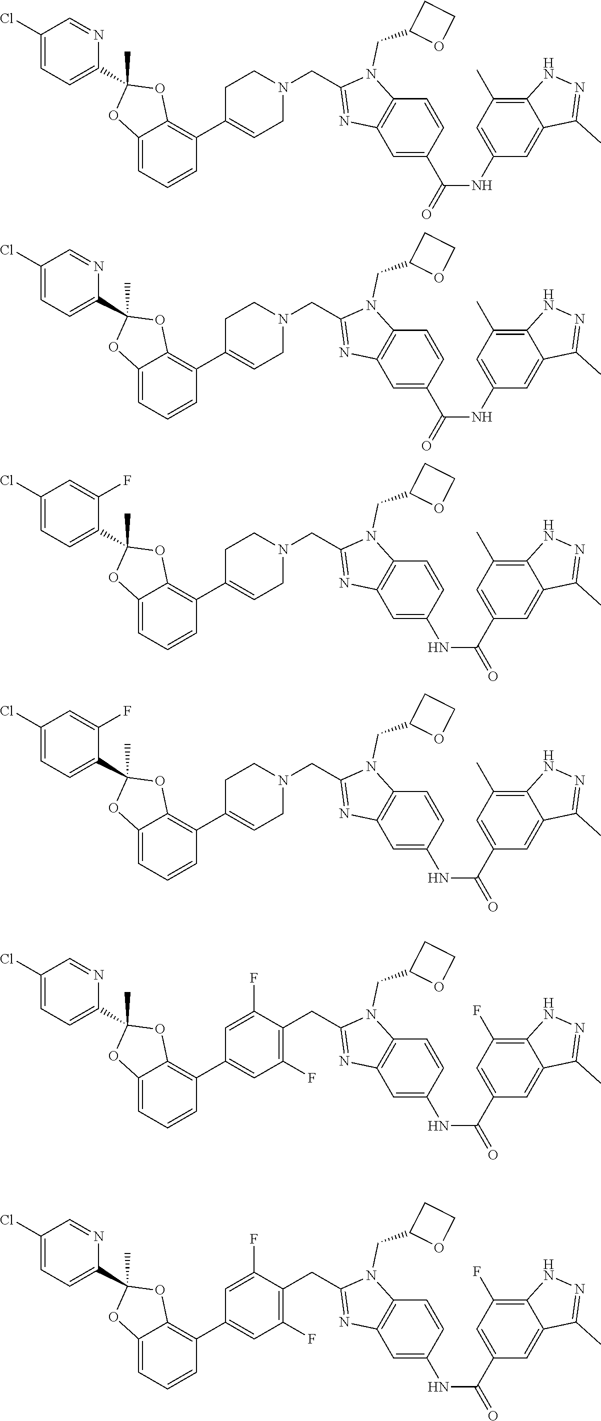

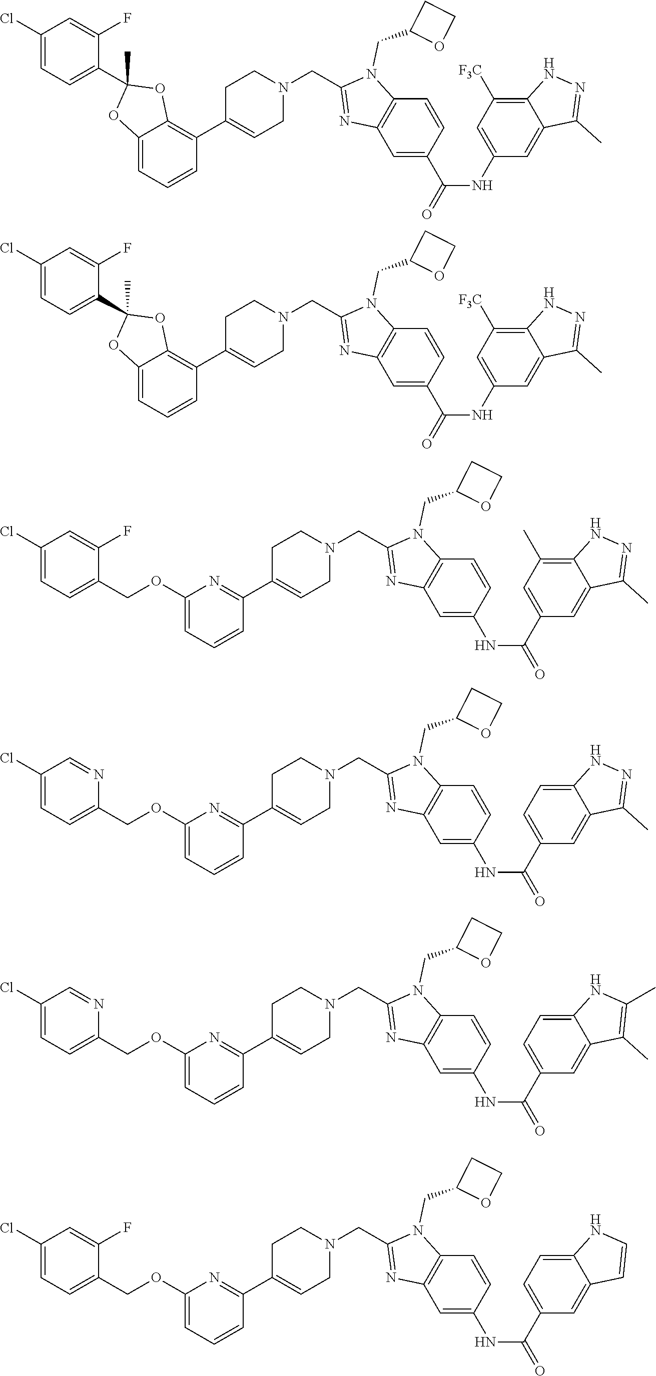

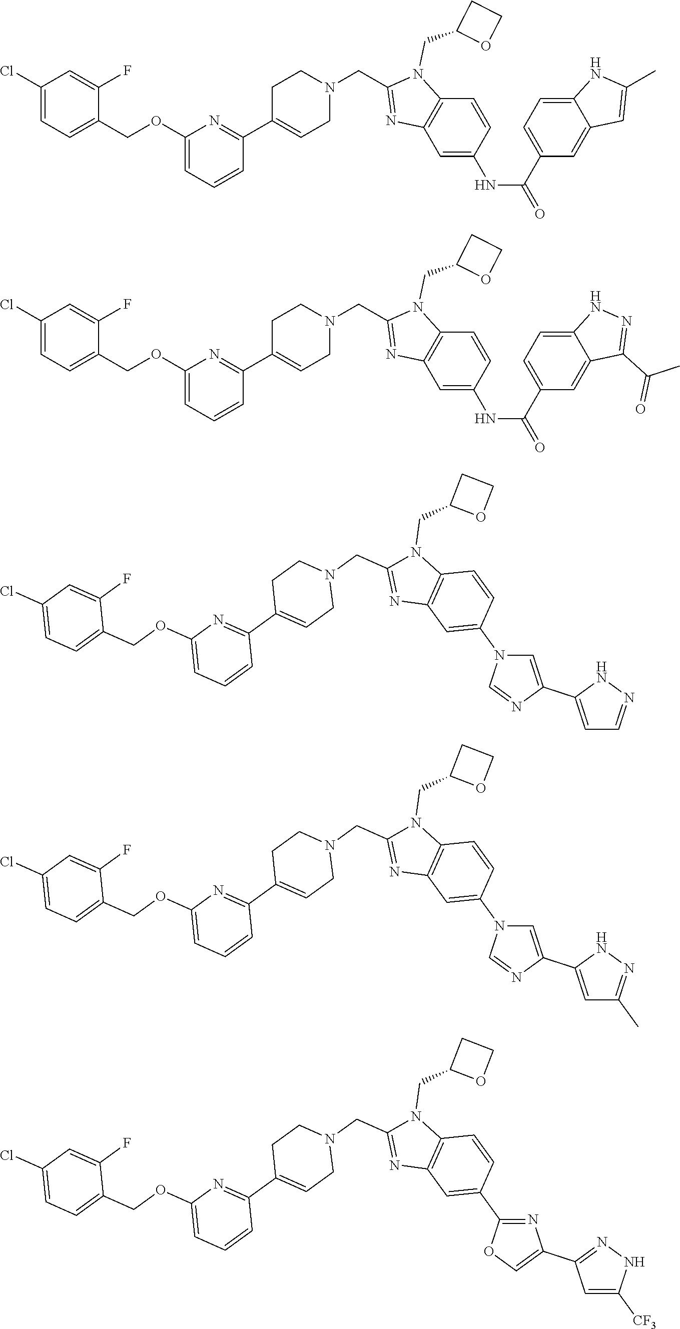 [SMILES: Cc1cc2cc(C(=O)Nc3ccc4c(c3)nc(CN3CC=C(c5cccc(OCc6ccc(Cl)cc6F)n5)CC3)n4C[C@@H]3CCO3)ccc2[nH]1 ; CC(=O)c1n[nH]c2ccc(C(=O)Nc3ccc4c(c3)nc(CN3CC=C(c5cccc(OCc6ccc(Cl)cc6F)n5)CC3)n4C[C@@H]3CCO3)cc12 ; Fc1cc(Cl)ccc1COc1cccc(C2=CCN(Cc3nc4cc(-n5cnc(-c6ccn[nH]6)c5)ccc4n3C[C@@H]3CCO3)CC2)n1 ; Cc1cc(-c2cn(-c3ccc4c(c3)nc(CN3CC=C(c5cccc(OCc6ccc(Cl)cc6F)n5)CC3)n4C[C@@H]3CCO3)cn2)[nH]n1 ; Fc1cc(Cl)ccc1COc1cccc(C2=CCN(Cc3nc4cc(-c5nc(-c6cc(C(F)(F)F)[nH]n6)co5)ccc4n3C[C@@H]3CCO3)CC2)n1]

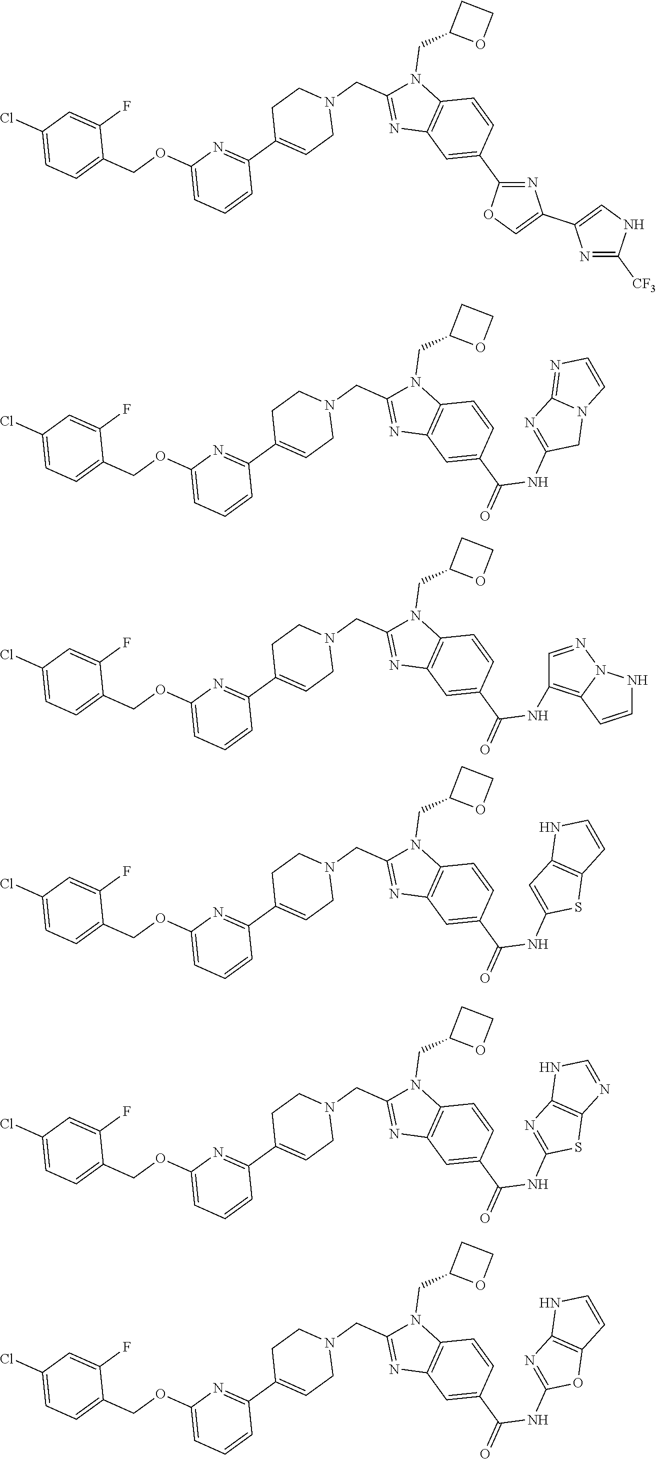

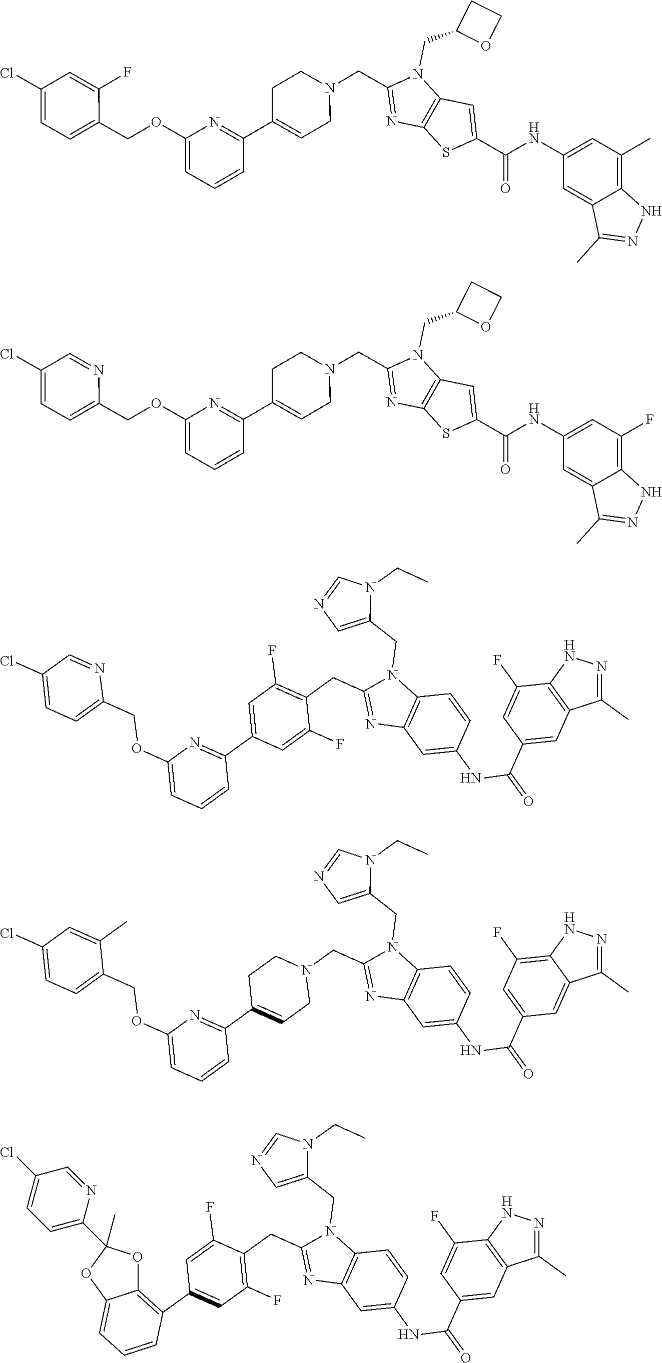 [SMILES: Cc1n[nH]c2c(C)cc(NC(=O)c3cc4c(nc(CN5CC=C(c6cccc(OCc7ccc(Cl)cc7F)n6)CC5)n4C[C@@H]4CCO4)s3)cc12 ; Cc1n[nH]c2c(F)cc(NC(=O)c3cc4c(nc(CN5CC=C(c6cccc(OCc7ccc(Cl)cn7)n6)CC5)n4C[C@@H]4CCO4)s3)cc12 ; CCn1cncc1Cn1c(Cc2c(F)cc(-c3cccc(OCc4ccc(Cl)cn4)n3)cc2F)nc2cc(NC(=O)c3cc(F)c4[nH]nc(C)c4c3)ccc21 ; CCn1cncc1Cn1c(CN2CC=C(c3cccc(OCc4ccc(Cl)cc4C)n3)CC2)nc2cc(NC(=O)c3cc(F)c4[nH]nc(C)c4c3)ccc21 ; CCn1cncc1Cn1c(Cc2c(F)cc(-c3cccc4c3OC(C)(c3ccc(Cl)cn3)O4)cc2F)nc2cc(NC(=O)c3cc(F)c4[nH]nc(C)c4c3)ccc21]

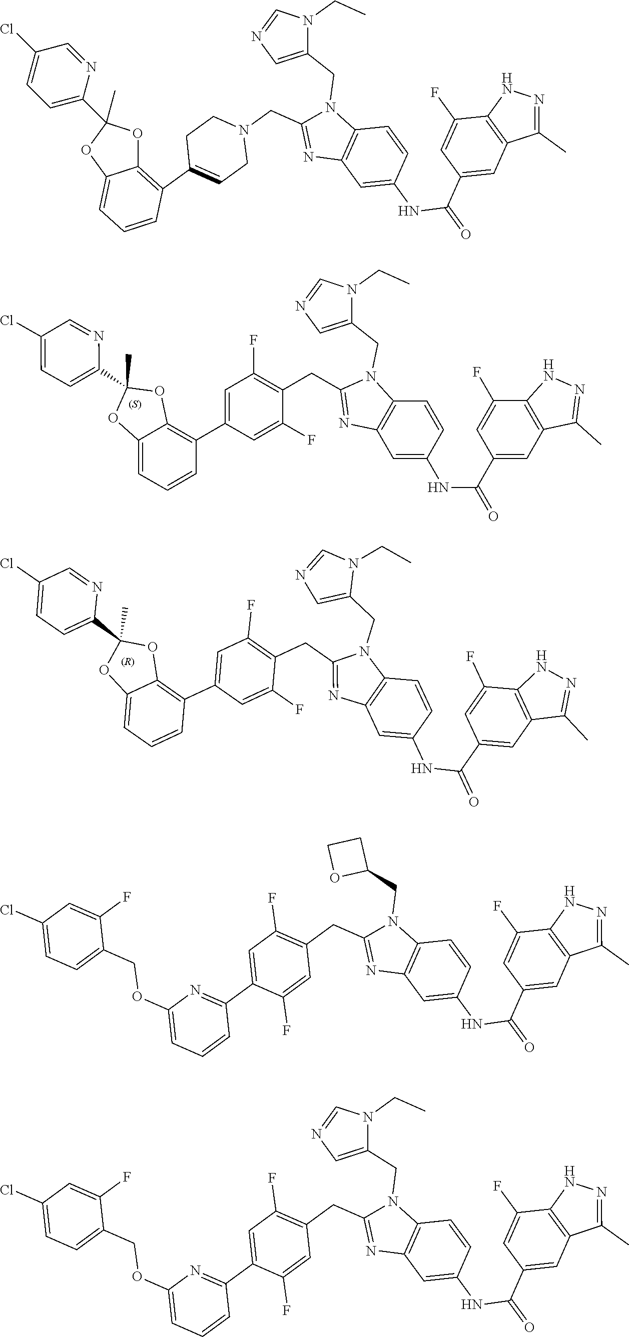

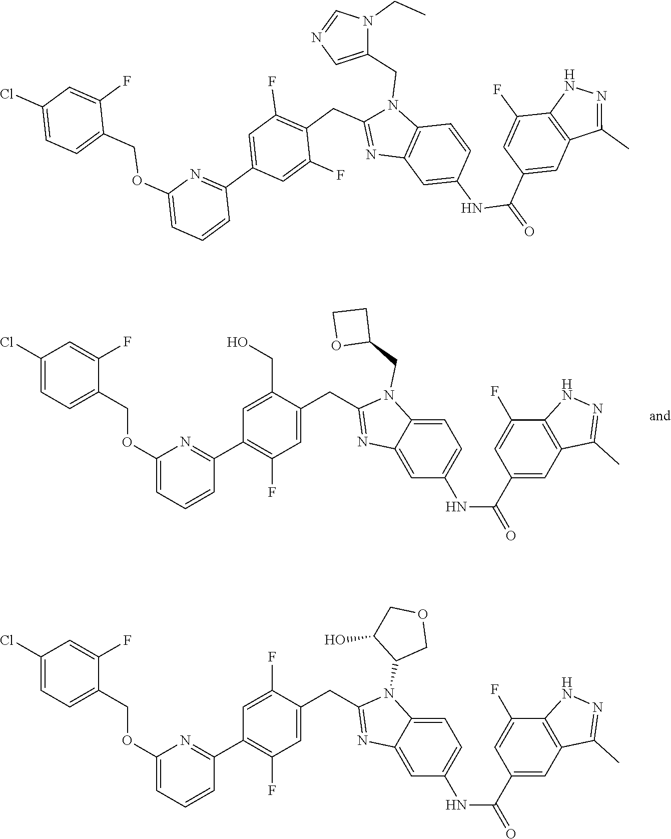

28. A pharmaceutical composition, comprising the compound of claim 1, or a pharmaceutically acceptable salt thereof, and a pharmaceutically acceptable excipient.

29. A method for treating a GLP-1R-mediated disease or condition, comprising the step of:
administering to a subject in need of such treatment a therapeutically effective amount of the compound of claim 1, or a pharmaceutically acceptable salt thereof, wherein the GLP-1R-mediated disease or condition is selected from the group consisting of T1D, T2DM, pre-diabetes, idiopathic T1D, LADA, EOD, YOAD, MODY, malnutrition-related diabetes, gestational diabetes, hyperglycemia, insulin resistance, hepatic insulin resistance, impaired glucose tolerance, diabetic neuropathy, diabetic nephropathy, kidney disease, diabetic retinopathy, adipocyte dysfunction, visceral adipose deposition, sleep apnea, obesity, eating disorders, weight gain from use of other agents, excessive sugar craving, dyslipidemia, hyperinsulinemia, NAFLD, NASH, fibrosis, cirrhosis, hepatocellular carcinoma, cardiovascular disease, atherosclerosis, coronary artery disease, peripheral vascular disease, hypertension, endothelial dysfunction, impaired vascular compliance, congestive heart failure, myocardial infarction, stroke, hemorrhagic stroke, ischemic stroke, traumatic brain injury, pulmonary hypertension, restenosis after angioplasty, intermittent claudication, post-prandial lipemia, metabolic acidosis, ketosis, arthritis, osteoporosis, Parkinson's Disease, left ventricular hypertrophy, peripheral arterial disease, macular degeneration, cataract, glomerulosclerosis, chronic renal failure, metabolic syndrome, syndrome X, premenstrual syndrome, angina pectoris, thrombosis, atherosclerosis, transient ischemic attacks, vascular restenosis, impaired glucose metabolism, conditions of impaired fasting plasma glucose, hyperuricemia, gout, erectile dysfunction, skin and connective tissue disorders, psoriasis, foot ulcerations, ulcerative colitis, hyper apo B lipoproteinemia, Alzheimer's disease, schizophrenia, impaired cognition, inflammatory bowel disease, short bowel syndrome, Crohn's disease, colitis, irritable bowel syndrome, polycystic ovary syndrome and substance addiction.
